(19) United States
(12) Patent Application Publication
(10) Pub. No.: US 2025/0325687 A1
(43) Pub. Date: Oct. 23, 2025

(54) CONJUGATE COMPRISING TOLL-LIKE RECEPTOR AGONIST

(71) Applicant: MABSOFT THERAPEUTICS (SHANGHAI) CO., LTD., Shanghai (CN)

(72) Inventors: Lianyong CHEN, Shanghai (CN); Yong ZHENG, Shanghai (CN); Ning ZHANG, Shanghai (CN)

(21) Appl. No.: 18/856,634

(22) PCT Filed: Apr. 14, 2023
(86) PCT No.: PCT/CN2023/088424

§ 371 Date: Oct. 14, 2024

(30) Foreign Application Priority Data
Apr. 15, 2022 (WO) PCT/CN2022/087052

(51) Int. Cl. A61K 47/68; A61K 31/437; A61P 31/14; C07D 471/04

(52) U.S. Cl. CPC A61K 47/6803; A61K 31/437; A61K 47/6851; A61K 47/6889; A61P 31/14; C07D 471/04

(57) ABSTRACT

Disclosed herein are compounds that are Toll-like receptor (TLR) agonist, conjugates comprising these compounds and cell targeting moiety, pharmaceutical compositions thereof, method of activating toll-like receptors 7 and/or 8 and method for the treatment of diseases or disorders mediated by toll-like receptors 7 and/or 8, in particular viral infections and proliferative disorders, such as cancer.

### TECHNICAL FIELD

The present disclosure generally relates to compounds that are Toll-like receptor (TLR) agonist, conjugates comprising these compounds, pharmaceutical compositions thereof, method for activating toll-like receptors 7 and/or 8 and method for the treatment of diseases or disorders mediated by toll-like receptors 7 and/or 8, in particular viral infections and proliferative disorders, such as cancer.

### BACKGROUND OF THE DISCLOSURE

Toll-like receptors (TLRs) are important proteins that recognize pathogen-associated molecular patterns (PAMPs), sense and initiate innate immune responses, and promote the development of adaptive immune responses. In humans, more than 10 TLRs are considered to have significant functions. These include TLRs 1, 2, 4, 5 and 6, which are confined to the cell surface and TLRs 3, 7, 8 and 9, which are expressed in endosomes. Engagement of TLRs with their specific ligands activates two major signaling pathways that are mediated by the adaptor proteins myeloid differentiation primary response gene 88 (MyD88) or TIR-domain-containing adaptor-inducing interferon-β (TRIF). Signaling cascades mediated by these pathways leads to the activation of transcription factors such as nuclear factor-kappa-B (NF-κB), activating protein-1 (AP-1) and interferon regulatory factors (IRFs) leading to the transcription of various genes for the production of inflammatory and anti-inflammatory cytokines, chemokines, and co-stimulatory molecules. Thus, the engagement of TLRs with their specific ligands leads to the activation of innate immune responses, and evokes adaptive immune response through the activation of antigen presenting cells (APCs) and by amplifying B- and T-cell effector cells.

One benefit of TLR7/8 agonists as immune response enhancers is their simultaneous stimulation of several cell types. TLR7 and TLR8 are expressed mostly on immune cell such as antigen presenting cells, including plasmacytoid dendritic cells (pDC) and myeloid dendritic cells (mDC), as well as natural killer cells and macrophages. TLR7/8 activation on pDCs and mDCs results in induction and release of type I interferons (IFN), tumor necrosis factor alpha (TNFα), and interleukin 12 (IL-12), which is important step for the initiation of innate and adaptive immunities to kill cancer cells. TLR7 and TLR8 also play a major role in the anti-viral response during viral infection by their ability to recognize single stranded RNA PAMPs. Accordingly, there is a need to develop small molecule agonists of TLR7 and TLR8 as both antiviral and anticancer compounds.

Cell binding agent-drug conjugates, including antibody-drug conjugates (ADC) are emerging as a powerful class of agents with efficacy cross a range of abnormal cell growth or proliferative diseases or disorders (e.g., cancers). Cell binding agent-drug conjugates (such as ADCs) are commonly composed of three distinct elements: a targeting moiety, a linker and a payload unit. Accordingly, there is also a need for TLR agonist conjugate to enhance the bioavailability, target-delivery and efficacy.

### SUMMARY OF THE DISCLOSURE

The present disclosure relates to compounds which are capable of activating toll-like receptors 7 and/or 8, the linker-payload compounds and the ADCs comprising these compounds, and the use of such compounds or ADCs for treatment of cancers or viral infections.

In one aspect, the present disclosure is directed to a conjugate compound having Formula (I):

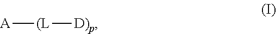

or a pharmaceutically acceptable salt thereof, wherein
A is a targeting moiety;
L is a linker;
p is an integer from 1 to 8;
D is a payload unit of Formula (II):

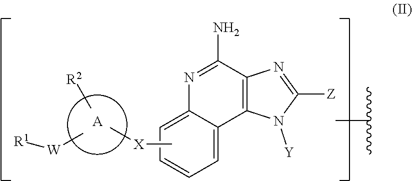

wherein:
   X is selected from the group consisting of —O—, —S—, —NH—, —(CH₂)ᵢ—, —(X¹)NC(O)—, —(X¹)NS(O)₂—, —C(O)N(X¹)— and —S(O)₂N(X¹)—, wherein —NH— and —(CH₂)ᵢ— are optionally substituted with one or more groups independently selected from halogen, hydroxyl, cyano, amino or alkyl;
   each X¹ is independently hydrogen, alkyl, alkenyl, or haloalkyl;
   Ring A is cycloalkyl, heterocyclyl, aryl or heteroaryl;
   W is selected from the group consisting of a direct bond, alkyl, cycloalkyl, heterocyclyl, *—W¹-alkyl-, *-alkyl-W¹—, and *-alkyl-W¹-alkyl-, wherein the alkyl, cycloalkyl, heterocyclyl, and alkyl in *—W¹-alkyl-, *-alkyl-W¹—, and *-alkyl-W¹-alkyl are optionally substituted with one or more groups independently selected from halogen, hydroxyl, cyano, amino, alkyl and —C(O)OR^{a}, and wherein * end of W is connected to Ring A;
   W¹ is —O—, —NR^{a}—, —C(O)—, —C(O)NR^{a}—, —OC(O)NR^{a}—, —NR^{a}C(O)—, —NR^{a}C(O)O—, or —NR^{a}C(O)NR^{a}—,
      each R^{a} is independently hydrogen, alkyl, or haloalkyl;
   R¹ is hydrogen, —N(R^{b})₂, hydroxyl or SH;
      each R^{b} is independently hydrogen, alkyl, or haloalkyl, or
      two R^{b} taken together with the nitrogen atom to which they are bound form a heterocyclyl;
   R² is hydrogen, halogen, hydroxyl, cyano, amino, alkyl, alkenyl, alkynyl, alkoxyl, haloalkyl, —OR^{e}, —OC(O)R^{e}, —SR^{e}, —B(OH)₂, —NO₂, —CHO, —C(═NOR^{e})(R^{e}), —R²⁻¹—N(R^{e})₂, —R²⁻¹—N(R^{e})C(O)R^{e}, —R²⁻¹—N(R^{e})S(O)₂R^{e}, —R²⁻¹—N(R^{e})P(O)₂R^{e}, —R²⁻¹—C(O)OR^{e}, —R²⁻¹—C(O)N(R^{e})₂, —R²⁻¹—S(O)₂N(R^{e})₂, —R²⁻¹—P(O)₂N(R^{e})₂, —OC(O)NR^{e}, or —NC(O)NR^{e},
      each R²⁻¹ is independently null or alkyl;
      each R^{e} is independently hydrogen, alkyl, or haloalkyl;
   Y is —Y¹—Y²—Y³, wherein
      Y¹ is a direct bond or —(CH₂)ₘ-Q¹-(CH₂)ₙ—O—*, wherein —(CH₂)ₘ— and —(CH₂)ₙ— are optionally substituted with one or more groups independently selected from halogen, hydroxyl, alkyl or haloalkyl, and * end of Y¹ is connected to Y²;
      Y² is a direct bond or —(CH₂)ₛ-Q²-(CH₂)ₜ—NR^{c}—**, wherein —(CH₂)ₛ— and —(CH₂)ₜ— are optionally substituted with one or more groups independently selected from halogen, hydroxyl, alkyl or haloalkyl, and ** end of Y² is connect to Y³;
      Y³ is selected from the group consisting of hydrogen, alkyl, -alkyl-aryl, -alkyl-N(R^{b})₂, —C(O)-alkyl, —C(O)-alkyl-N(R^{b})₂, and —S(O)₂-alkyl;
      Q¹ and Q² are each independently selected from a direct bond, cycloalkyl, heterocyclyl, aryl, or heteroaryl, wherein the cycloalkyl, heterocyclyl, aryl, and heteroaryl are optionally substituted with one or more groups independently selected from halogen, hydroxyl, cyano, amino, or alkyl;
      R^{c} is hydrogen or alkyl, or
      R^{c} and Y³ taken together with the atoms to which they are bound form a heterocyclyl optionally substituted with one or more groups independently selected from halogen, hydroxyl, cyano, amino or alkyl;
   Z is selected from the group consisting of alkyl, alkenyl, alkynyl, heteroalkyl, heteroalkenyl and heteroalkynyl, each of which is optionally substituted with one or more R^{d};
      R^{d} is selected from the group consisting of halogen, acyl, alkyl, alkenyl, alkynyl, cycloalkyl and —O-cycloalkyl;
   i is 0, 1, 2, 3, 4, 5 or 6;
   m is 0, 1, 2, 3, 4 or 5;
   n is 0, 1, 2, 3, 4 or 5;
   s is 0, 1, 2, 3, 4 or 5; and
   t is 0, 1, 2, 3, 4 or 5.

In another aspect, there is provided a linker-payload compound having Formula (Ia):

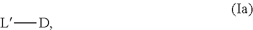

or a pharmaceutically acceptable salt thereof, wherein
L′ is a linker precursor;
D is a payload unit of Formula (II):

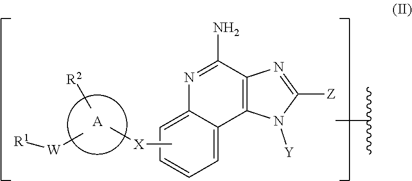

wherein:
   X is selected from the group consisting of —O—, —S—, —NH—, —(CH₂)ᵢ—, —(X¹)NC(O)—, —(X¹)NS(O)₂—, —C(O)N(X¹)— and —S(O)₂N(X¹)—, wherein —NH— and —(CH₂)ᵢ— are optionally substituted with one or more groups independently selected from halogen, hydroxyl, cyano, amino or alkyl;
   each X¹ is independently hydrogen, alkyl, alkenyl, or haloalkyl;
   Ring A is cycloalkyl, heterocyclyl, aryl or heteroaryl;
   W is selected from the group consisting of a direct bond, alkyl, cycloalkyl, heterocyclyl, *—W¹-alkyl-, *-alkyl-W¹—, and *-alkyl-W¹-alkyl-, wherein the alkyl, cycloalkyl, heterocyclyl, and alkyl in *—W¹-alkyl-, *-alkyl-W¹—, and *-alkyl-W¹-alkyl are optionally substituted with one or more groups independently selected from halogen, hydroxyl, cyano, amino, alkyl and —C(O)OR^{a}, and wherein * end of W is connected to Ring A;
   W¹ is —O—, —NR^{a}—, —C(O)—, —C(O)NR^{a}—, —OC(O)NR^{a}—, —NR^{a}C(O)—, —NR^{a}C(O)O—, or —NR^{a}C(O)NR^{a}—,
      each R^{a} is independently hydrogen, alkyl, or haloalkyl;
   R¹ is hydrogen, —N(R^{b})₂, hydroxyl or SH;
      each R^{b} is independently hydrogen, alkyl, or haloalkyl, or
      two R^{b} taken together with the nitrogen atom to which they are bound form a heterocyclyl;
   R² is hydrogen, halogen, hydroxyl, cyano, amino, alkyl, alkenyl, alkynyl, alkoxyl, haloalkyl, —OR^{e}, —OC(O)R^{e}, —SR^{e}, —B(OH)₂, —NO₂, —CHO, —C(═NOR^{e})(R^{e}), —R²⁻¹—N(R^{e})₂, —R²⁻¹—N(R^{e})C(O)R^{e}, —R²⁻¹—N(R^{e})S(O)₂R^{e}, —R²⁻¹—N(R^{e})P(O)₂R^{e}, —R²⁻¹—C(O)OR^{e}, —R²⁻¹—C(O)N(R^{e})₂, —R²⁻¹—S(O)₂N(R^{e})₂, —R²⁻¹—P(O)₂N(R^{e})₂, —OC(O)NR^{e}, or —NC(O)NR^{e},
      each R²⁻¹ is independently null or alkyl;
   each R^{e} is independently hydrogen, alkyl, or haloalkyl; Y is —Y¹—Y²—Y³, wherein
      Y¹ is a direct bond or —(CH₂)ₘ-Q¹-(CH₂)ₙ—O—*, wherein * end of Y¹ is connected to Y²;
      Y² is a direct bond or —(CH₂)ₛ-Q²-(CH₂)ₜNR^{c}—**, wherein ** end of Y² is connect to Y³;
      Y³ is selected from the group consisting of hydrogen, alkyl, -alkyl-aryl, -alkyl-N(R^{b})₂, —C(O)-alkyl, —C(O)-alkyl-N(R^{b})₂, and —S(O)₂-alkyl;
      Q¹ and Q² are each independently selected from a direct bond, cycloalkyl, heterocyclyl, aryl, or heteroaryl, wherein the cycloalkyl, heterocyclyl, aryl, and heteroaryl are optionally substituted with one or more groups independently selected from halogen, hydroxyl, cyano, amino, or alkyl;
      R^{c} is hydrogen or alkyl, or
      R^{c} and Y³ taken together with the atoms to which they are bound form a heterocyclyl optionally substituted with one or more groups independently selected from halogen, hydroxyl, cyano, amino or alkyl;
   Z is selected from the group consisting of alkyl, alkenyl, alkynyl, heteroalkyl, heteroalkenyl and heteroalkynyl, each of which is optionally substituted with one or more R^{d};
   R^{d} is selected from the group consisting of halogen, acyl, alkyl, alkenyl, alkynyl, cycloalkyl and —O-cycloalkyl;
i is 0, 1, 2, 3, 4, 5 or 6;
m is 0, 1, 2, 3, 4 or 5;
n is 0, 1, 2, 3, 4 or 5;
s is 0, 1, 2, 3, 4 or 5; and
t is 0, 1, 2, 3, 4 or 5.

In another aspect, there is provided a payload compound having Formula (II′):

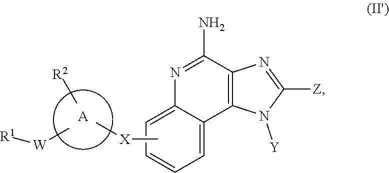

or a pharmaceutically acceptable salt thereof, wherein:
   X is selected from the group consisting of —O—, —S—, —NH—, —(CH₂)ᵢ—, —(X¹)NC(O)—, —(X¹)NS(O)₂—, —C(O)N(X¹)— and —S(O)₂N(X¹)—, wherein —NH— and —(CH₂)ᵢ— are optionally substituted with one or more groups independently selected from halogen, hydroxyl, cyano, amino or alkyl;
   each X¹ is independently hydrogen, alkyl, alkenyl, or haloalkyl;
   Ring A is cycloalkyl, heteroalkyl, aryl or heteroaryl;
   W is selected from the group consisting of a direct bond, alkyl, cycloalkyl, heterocyclyl, *—W¹-alkyl-, *-alkyl-W¹—, and *-alkyl-W¹-alkyl-, wherein the alkyl, cycloalkyl, heterocyclyl, and alkyl in *—W¹-alkyl-, *-alkyl-W¹—, and *-alkyl-W¹-alkyl are optionally substituted with one or more groups independently selected from halogen, hydroxyl, cyano, amino, alkyl and —C(O)OR^{a}, and wherein * end of W is connected to Ring A;
   W¹ is —O—, —NR^{a}—, —C(O)—, —C(O)NR^{a}—, —OC(O)NR^{a}—, —NR^{a}C(O)—, —NR^{a}C(O)O—, or —NR^{a}C(O)NR^{a}—,
      each R^{a} is independently hydrogen, alkyl, or haloalkyl;
   R¹ is hydrogen, —N(R^{b})₂, hydroxyl or SH;
      each R^{b} is independently hydrogen, alkyl, or haloalkyl, or
      two R^{b} taken together with the nitrogen atom to which they are bound form a heterocyclyl;
   R² is hydrogen, halogen, hydroxyl, cyano, amino, alkyl, alkenyl, alkynyl, alkoxyl, haloalkyl, —OR^{e}, —OC(O)R^{e}, —SR^{e}, —B(OH)₂, —NO₂, —CHO, —C(═NOR^{e})(R^{e}), —R²⁻¹—N(R^{e})₂, —R²⁻¹—N(R^{e})C(O)R^{e}, —R²⁻¹—N(R^{e})S(O)₂R^{e}, —R²⁻¹—N(R^{e})P(O)₂R^{e}, —R²⁻¹—C(O)OR^{e}, —R²⁻¹—C(O)N(R^{e})₂, —R²⁻¹—S(O)₂N(R^{e})₂, —R²⁻¹—P(O)₂N(R^{e})₂, —OC(O)NR^{e}, or —NC(O)NR^{e},
      each R²⁻¹ is independently null or alkyl;
      each R^{e} is independently hydrogen, alkyl, or haloalkyl;
   Y is —Y¹—Y²—Y³, wherein
      Y¹ is a direct bond or —(CH₂)ₘ-Q¹-(CH₂)ₙ—O—*, wherein * end of Y¹ is connected to Y²;
      Y² is a direct bond or —(CH₂)ₛ-Q²-(CH₂)ₜ—NR^{c}—**, wherein ** end of Y² is connect to Y³;
      Y³ is selected from the group consisting of hydrogen, alkyl, -alkyl-aryl, -alkyl-N(R^{b})₂, —C(O)-alkyl, —C(O)-alkyl-N(R^{b})₂, and —S(O)₂-alkyl;
      Q¹ and Q² are each independently selected from a direct bond, cycloalkyl, heterocyclyl, aryl, or heteroaryl, wherein the cycloalkyl, heterocyclyl, aryl, and heteroaryl are optionally substituted with one or more groups independently selected from halogen, hydroxyl, cyano, amino, or alkyl;
      R^{c} is hydrogen or alkyl, or
      R^{c} and Y³ taken together with the atoms to which they are bound form a heterocyclyl optionally substituted with one or more groups independently selected from halogen, hydroxyl, cyano, amino or alkyl;
   Z is selected from the group consisting of alkyl, alkenyl, alkynyl, heteroalkyl, heteroalkenyl and heteroalkynyl, each of which is optionally substituted with one or more R^{d};
      R^{d} is selected from the group consisting of halogen, acyl, alkyl, alkenyl, alkynyl, cycloalkyl and —O-cycloalkyl;
   i is 0, 1, 2, 3, 4, 5 or 6;
   m is 0, 1, 2, 3, 4 or 5;
   n is 0, 1, 2, 3, 4 or 5;
   s is 0, 1, 2, 3, 4 or 5; and
   t is 0, 1, 2, 3, 4 or 5.

In a further aspect, there is provided a payload compound having Formula (IIa′):

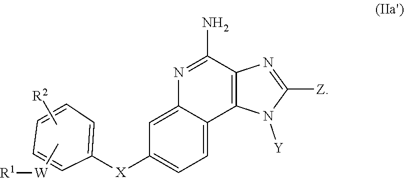

In another aspect, the present disclosure is directed to pharmaceutical composition comprising one or more conjugate compounds, linker-payload compounds or payload compounds of the present disclosure, and one or more pharmaceutically acceptable carriers.

In a further aspect, the present disclosure is directed to methods for treating a disease mediated by toll-like receptors 7 and/or 8 in a subject in need thereof, comprising administering an effective amount of the one or more conjugate compounds, linker-payload compounds or payload compounds of the present disclosure or a pharmaceutically acceptable salt thereof or the pharmaceutical composition of the present disclosure to the subject.

In a further aspect, the present disclosure is directed to methods for activating toll-like receptors 7 and/or 8 in a subject in need thereof, comprising administering one or more conjugate compounds, linker-payload compounds or payload compounds of the present disclosure or the pharmaceutical composition of the present disclosure to the subject.

In a further aspect, the present disclosure is directed to method for stimulating an immune response in a subject in need thereof, comprising administering one or more conjugate compounds, linker-payload compounds or payload compounds of the present disclosure or the pharmaceutical composition of the present disclosure to the subject.

In another aspect, the present disclosure is directed to use of one or more conjugate compounds, linker-payload compounds or payload compounds of the present disclosure or the pharmaceutical composition of the present disclosure, in the manufacture of a medicament for treating viral infections or cancers.

### BRIEF DESCRIPTION OF THE DRAWINGS

FIGS. 1(a) and 1(b) show the DAR measurement by MS method of an exemplary conjugate comprising payload compound 16.

FIG. 2(a) shows the induction of TNF-α production of conjugates 1-5, and control anti-Her2 antibody (Herceptin) by Balb/c mouse BMDM (Bone Marrow Derived Macrophages) in the presence of tumor cells.

FIG. 2(b) shows the induction of TNF-α production of conjugates 6, 8, 9, 11, and control anti-Her2 antibody (Herceptin) by Balb/c mouse BMDM (Bone Marrow Derived Macrophages) in the presence of tumor cells.

FIG. 2(c) shows the induction of TNF-α production of conjugates 7, 10, 12, 13, and control anti-Her2 antibody (Herceptin) by Balb/c mouse BMDM (Bone Marrow Derived Macrophages) in the presence of tumor cells.

FIG. 2(d) shows the induction of TNF-α production of conjugates 9, 13, 14, 26, 27, and control anti-Her2 antibody (Herceptin) by Balb/c mouse BMDM (Bone Marrow Derived Macrophages) in the presence of tumor cells.

FIG. 2(e) shows the induction of TNF-α production of conjugates 13, 16, 18, 19, 28, and control anti-Her2 antibody (Herceptin) by Balb/c mouse BMDM (Bone Marrow Derived Macrophages) in the presence of tumor cells.

FIG. 2(f) shows the induction of TNF-α production of conjugates 13, 21, 23, 25, 29, and control anti-Her2 antibody (Herceptin) by Balb/c mouse BMDM (Bone Marrow Derived Macrophages) in the presence of tumor cells.

FIG. 2(g) shows the induction of TNF-α production of conjugates 13, 17, 36, 37, 39, and control anti-Her2 antibody (Herceptin) by Balb/c mouse BMDM (Bone Marrow Derived Macrophages) in the presence of tumor cells.

FIG. 2(h) shows the induction of TNF-α production of conjugates 17, 20, 22, 24, and control anti-Her2 antibody (Herceptin) by Balb/c mouse BMDM (Bone Marrow Derived Macrophages) in the presence of tumor cells.

FIG. 2(i) shows the induction of TNF-α production of conjugates 30, 31, 32, 35, and control anti-Her2 antibody (Herceptin) by Balb/c mouse BMDM (Bone Marrow Derived Macrophages) in the presence of tumor cells.

FIG. 2(j) shows the induction of TNF-α production of conjugates 15, 33, 34, 38, 41, and control anti-Her2 antibody (Herceptin) by Balb/c mouse BMDM (Bone Marrow Derived Macrophages) in the presence of tumor cells.

FIG. 2(k) shows the induction of TNF-α production of conjugates 31, 40, 42, 43, and control anti-Her2 antibody (Herceptin) by Balb/c mouse BMDM (Bone Marrow Derived Macrophages) in the presence of tumor cells.

FIGS. 3(a) and 3(b) show the anti-tumor efficacy of Conjugates 1-2 against EMT6-hHER2 model in Balb/c mice compared with vehicle control group.

FIGS. 4(a) and 4(b) show the anti-tumor efficacy of Conjugates 4-5 against EMT6-hHER2 model in Balb/c mice compared with vehicle control group.

FIGS. 5(a) and 5(b) show the anti-tumor efficacy of Conjugates 6-9 against EMT6-hHER2 model in Balb/c mice compared with vehicle control group.

FIGS. 6(a) and 6(b) show the anti-tumor efficacy of Conjugates 10-13 against EMT6-hHER2 model in Balb/c mice compared with vehicle control group.

FIGS. 7(a) and 7(b) show the anti-tumor efficacy of Conjugates 9, 13, 14, 16, 18, 19, 21, 23, 25, 26, 27, 28, and 29 against HCC1954 model in SCID Beige mice compared with vehicle control group.

FIGS. 8(a) and 8(b) show the anti-tumor efficacy of Conjugates 15, 17, 20, 22 and 24 (5 mg/kg, i.v.) against HCC1954 model in SCID Beige mice compared with vehicle control group.

FIGS. 9(a) and 9(b) show the anti-tumor efficacy of Conjugates 15, 17, 20, 22 and 24 (2.5 mg/kg, i.v.) against HCC1954 model in SCID Beige mice compared with vehicle control group.

### DETAILED DESCRIPTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the present invention as defined by the claims. One skilled in the art will recognize many methods and materials similar or equivalent to those described herein, which could be used in the practice of the present invention. The present invention is in no way limited to the methods and materials described. In the event that one or more of the incorporated literature and similar materials differs from or contradicts this application, including but not limited to defined terms, term usage, described techniques, or the like, this application controls.

It is appreciated that certain features of the present disclosure, which are, for clarity, described in the context of separate embodiments, can also be provided in combination in a single embodiment. Conversely, various features of the present disclosure, which are, for brevity, described in the context of a single embodiment, can also be provided separately or in any suitable sub-combination.

It must be noted that, as used in the specification and the appended claims, the singular forms “a,” “an,” and “the” include plural forms of the same unless the context clearly dictates otherwise. Thus, for example, reference to “a compound” includes a plurality of compounds. In this specification and in the claims that follow, reference will be made to a number of terms that shall be defined to have the following meanings unless a contrary intention is apparent.

### Definitions

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

At various places in the present disclosure, linking substituents are described. Where the structure clearly requires a linking group, the Markush variables listed for that group are understood to be linking groups. For example, if the structure requires a linking group and the Markush group definition for that variable lists “alkyl”, then it is understood that the “alkyl” represents a linking alkylene group.

When any variable (e.g., Rⁱ) occurs more than one time in any constituent or formula for a compound, its definition at each occurrence is independent of its definition at every other occurrence. Thus, for example, if a group is shown to be substituted with 0-2 Rⁱ moieties, then the group may optionally be substituted with up to two Rⁱ moieties and Rⁱ at each occurrence is selected independently from the definition of Rⁱ. Also, combinations of substituents and/or variables are permissible, but only if such combinations result in stable compounds.

As used herein, a dash “-” at the front or end of a chemical group is used, a matter of convenience, to indicate a point of attachment for a substituent. For example, —OH is attached through the carbon atom; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named. As used herein, a solid line coming out of the center of a ring indicates that the point of attachment for a substituent on the ring can be at any ring atom. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., Rⁱ) occurs more than one time in any constituent or formula for a compound, its definition at each occurrence is independent of its definition at every other occurrence. Thus, for example, if a group is shown to be substituted with 0-2 Rⁱ moieties, then the group may optionally be substituted with up to two Rⁱ moieties and Rⁱ at each occurrence is selected independently from the definition of Rⁱ. Also, combinations of substituents and/or variables are permissible, but only if such combinations result in stable compounds.

As used herein, the term “about”, directed to that value or parameter per se, includes the indicated amount ±10%, ±5%, or ±10%. Also, the term “about X” includes description of “X”.

As used herein, the term “compounds provided herein”, or “compounds disclosed herein” or “compounds of the present disclosure” refers to the compounds of Formula (I), Formula (Ta), Formula (II), Formula (IIa), Formula (II′), Formula (IIa′), as well as the specific compounds disclosed herein.

As used herein, the term “Cᵢ₋ⱼ” indicates a range of the carbon atoms numbers, wherein i and j are integers and the range of the carbon atoms numbers includes the endpoints (i.e. i and j) and each integer point in between, and wherein j is greater than i. For examples, C₁₋₆ indicates a range of one to six carbon atoms, including one carbon atom, two carbon atoms, three carbon atoms, four carbon atoms, five carbon atoms and six carbon atoms. In some embodiments, the term “C₁₋₁₂” indicates 1 to 12, particularly 1 to 10, particularly 1 to 8, particularly 1 to 6, particularly 1 to 5, particularly 1 to 4, particularly 1 to 3 or particularly 1 to 2 carbon atoms. In similar manner, the term “m-n membered” ring, wherein m and n are integers and n is greater than m, refers to a ring containing m to n atoms.

As used herein, the term “alkyl”, whether as part of another term or used independently, refers to a saturated linear or branched-chain hydrocarbon radical, which may be optionally substituted independently with one or more substituents described below. The term “Cᵢ₋ⱼ alkyl” refers to a linear or branched-chain alkyl having i to j carbon atoms. For example, alkyl groups contain 1 to 10, 1 to 9, 1 to 8, 1 to 7, 1 to 6, 1 to 5, 1 to 4, 1 to 3, or 1 to 2 carbon atoms. Examples of “C₁₋₆ alkyl” include, but are not limited to, methyl, ethyl, propyl, isopropyl, n-butyl, i-butyl, s-butyl, t-butyl, n-pentyl, 2-pentyl, 3-pentyl, 2-methyl-2-butyl, 3-methyl-2-butyl, 3-methyl-1-butyl, 2-methyl-1-butyl, 2-ethyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, and the like.

As used herein, the term “alkylaryl” refers to an alkyl attached to aryl, including -alkyl-aryl and alkyl-aryl-. In some embodiments, alkylaryl refers to -alkyl-aryl.

As used herein, the term “alkylcycloalkyl” refers to an alkyl attached to cycloalkyl, including -alkyl-cycloalkyl and alkyl-cycloalkyl-. In some embodiments, alkylcycloalkyl refers to -alkyl-cycloalkyl.

As used herein, the term “alkylheterocyclyl” refers to an alkyl attached to heterocyclyl, including -alkyl-heterocyclyl and alkyl-heterocyclyl-. In some embodiments, alkylheterocyclyl refers to -alkyl-heterocyclyl.

As used herein, the term “alkenyl”, whether as part of another term or used independently, refers to linear or branched-chain hydrocarbon radical having at least one carbon-carbon double bond, which may be optionally substituted independently with one or more substituents described herein, and includes radicals having “cis” and “trans” orientations, or alternatively, “E” and “Z” orientations. In some embodiments, alkenyl groups contain 2 to 12 carbon atoms. In some embodiments, alkenyl groups contain 2 to 11 carbon atoms. In some embodiments, alkenyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkenyl groups contain 2 carbon atoms. Examples of alkenyl group include, but are not limited to, ethylenyl (or vinyl), propenyl (allyl), butenyl, pentenyl, 1-methyl-2 buten-1-yl, 5-hexenyl, and the like.

As used herein, the term “alkynyl”, whether as part of another term or used independently, refers to a linear or branched hydrocarbon radical having at least one carbon-carbon triple bond, which may be optionally substituted independently with one or more substituents described herein. In some embodiments, alkenyl groups contain 2 to 12 carbon atoms. In some embodiments, alkynyl groups contain 2 to 11 carbon atoms. In some embodiments, alkynyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkynyl groups contain 2 carbon atoms. Examples of alkynyl group include, but are not limited to, ethynyl, 1-propynyl, 2-propynyl, and the like.

As used herein, the term “alkoxyl”, whether as part of another term or used independently, refers to an alkyl group, as previously defined, attached to the parent molecule through an oxygen atom. The term “Cᵢ₋ⱼ alkoxyl” means that the alkyl moiety of the alkoxyl group has i to j carbon atoms. For example, alkoxy groups can contain 1 to 10, 1 to 9, 1 to 8, 1 to 7, 1 to 6, 1 to 5, 1 to 4, 1 to 3, or 1 to 2 carbon atoms. Examples of “C₁₋₆ alkoxyl” include, but are not limited to, methoxy, ethoxy, propoxy (e.g. n-propoxy and isopropoxy), t-butoxy, neopentoxy, n-hexoxy, and the like.

As used herein, the term “amino” refers to the group —NR^{a}R^{b}, wherein R^{a} and R^{b} are independently selected from groups consisting of hydrogen, alkyl, haloalkyl, aryl, heteroaryl, cycloalkyl, and heterocyclyl and each of which may be optionally substituted.

As used herein, the term “aryl”, whether as part of another term or used independently, refers to monocyclic and polycyclic ring systems having a total of 5 to 20 ring members, wherein at least one ring in the system is aromatic and wherein each ring in the system contains 3 to 12 ring members. Examples of “aryl” include, but are not limited to, phenyl, biphenyl, naphthyl, anthracyl and the like, which may bear one or more substituents. Also included within the scope of the term “aryl”, as it is used herein, is a group in which an aromatic ring is fused to one or more additional rings. In the case of polycyclic ring system, only one of the rings needs to be aromatic (e.g., 2,3-dihydroindole), although all of the rings may be aromatic (e.g., quinoline). The second ring can also be fused or bridged. Examples of polycyclic aryl include, but are not limited to, benzofuranyl, indanyl, phthalimidyl, naphthimidyl, phenanthridinyl, or tetrahydronaphthyl, and the like.

As used herein, the term “arylalkyl” refers to an aryl attached to alkyl, including -aryl-alkyl and aryl-alkyl-. In some embodiments, arylalkyl refers to -aryl-alkyl.

As used herein, the term “cyano” refers to —CN.

As used herein, the term “cycloalkyl”, whether as part of another term or used independently, refers to a monovalent non-aromatic, saturated or partially unsaturated monocyclic and polycyclic ring system, in which all the ring atoms are carbon and which contains at least three ring forming carbon atoms. In some embodiments, the cycloalkyl group may contain 3 to 12 ring forming carbon atoms, 3 to 10 ring forming carbon atoms, 3 to 9 ring forming carbon atoms, 3 to 8 ring forming carbon atoms, 3 to 7 ring forming carbon atoms, 3 to 6 ring forming carbon atoms, 3 to 5 ring forming carbon atoms, 4 to 12 ring forming carbon atoms, 4 to 10 ring forming carbon atoms, 4 to 9 ring forming carbon atoms, 4 to 8 ring forming carbon atoms, 4 to 7 ring forming carbon atoms, 4 to 6 ring forming carbon atoms, 4 to 5 ring forming carbon atoms. The cycloalkyl group may be saturated or partially unsaturated. In some embodiments, the cycloalkyl group may be a saturated cyclic alkyl group. In some embodiments, the cycloalkyl group may be a partially unsaturated cyclic alkyl group that contains at least one double bond or triple bond in its ring system.

In some embodiments, the cycloalkyl group may be saturated or partially unsaturated monocyclic carbocyclic ring system, examples of which include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl and cyclododecyl.

In some embodiments, the cycloalkyl group may be saturated or partially unsaturated polycyclic (e.g., bicyclic and tricyclic) carbocyclic ring system, which can be arranged as a fused, spiro or bridged ring system. As used herein, the term “fused ring” refers to a ring system having two rings sharing two adjacent atoms, the term “spiro ring” refers to a ring systems having two rings connected through one single common atom, and the term “bridged ring” refers to a ring system with two rings sharing three or more atoms. Examples of fused carbocyclyl include, but are not limited to, naphthyl, benzopyrenyl, anthracenyl, acenaphthenyl, fluorenyl and the like. Examples of spiro carbocyclyl include, but are not limited to, spiro[5.5]undecanyl, spiro-pentadienyl, spiro[3.6]-decanyl, and the like. Examples of bridged carbocyclyl include, but are not limited to bicyclo[1,1,1]pentenyl, bicyclo[2,2,1]heptenyl, bicyclo[2.2.1]heptanyl, bicyclo[2.2.2]octanyl, bicyclo[3.3.1]nonanyl, bicyclo[3.3.3]undecanyl, and the like.

As used herein, the term “cycloalkylalkyl” refers to a cycloalkyl attached to alkyl, including -cycloalkyl-alkyl and cycloalkyl-alkyl-. In some embodiments, cycloalkylalkyl refers to -cycloalkyl-alkyl.

As used herein, the term “halo” or “halogen” refers to an atom selected from fluorine (or fluoro), chlorine (or chloro), bromine (or bromo) and iodine (or iodo).

As used herein, the term “haloalkyl” refers to an alkyl group as defined above, wherein one or more hydrogen atoms are replaced by a halogen. If a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Some examples of haloalkyl include difluoromethyl (—CHF₂) and trifluoromethyl (—CF₃).

As used herein, the term “heteroatom” refers to nitrogen, oxygen, sulfur or phosphorus, and includes any oxidized form of nitrogen, sulfur or phosphorus, and any quaternized form of a basic nitrogen.

As used herein, the term “heteroalkyl” refers to an alkyl, at least one of the carbon atoms of which is replaced with a heteroatom selected from N, O, or S. The heteroalkyl may be a carbon radical or heteroatom radical (i.e., the heteroatom may appear in the middle or at the end of the radical), and may be optionally substituted independently with one or more substituents described herein. The term “heteroalkyl” encompasses alkoxyl and heteroalkoxy radicals.

As used herein, the term “heteroalkenyl” refers to an alkenyl, at least one of the carbon atoms of which is replaced with a heteroatom selected from N, O, or S. The heteroalkenyl may be a carbon radical or heteroatom radical (i.e., the heteroatom may appear in the middle or at the end of the radical), and may be optionally substituted independently with one or more substituents described herein.

As used herein, the term “heteroalkynyl” refers to an alkynyl, at least one of the carbon atoms of which is replaced with a heteroatom selected from N, O, or S. The heteroalkynyl may be a carbon radical or heteroatom radical (i.e., the heteroatom may appear in the middle or at the end of the radical), and may be optionally substituted independently with one or more substituents described herein.

As used herein, the term “heteroaryl”, whether as part of another term or used independently, refers to an aryl group having, in addition to carbon atoms, one or more heteroatoms. The heteroaryl group can be monocyclic. Examples of monocyclic heteroaryl include, but are not limited to, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, benzofuranyl and pteridinyl. The heteroaryl group also includes polycyclic groups in which a heteroaromatic ring is fused to one or more aryl, heteroaryl, cycloaliphatic, or heterocyclyl rings, where the radical or point of attachment is on the heteroaromatic ring. Examples of polycyclic heteroaryl include, but are not limited to, indolyl, isoindolyl, benzothienyl, benzofuranyl, benzo[1,3]dioxolyl, dibenzofuranyl, indazolyl, benzimidazolyl, benzthiazolyl, quinolyl, isoquinolyl, dihydroquinolinyl, dihydroisoquinolinyl, tetrahydroquinolinyl, tetrahydroisoquinolinyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and the like.

As used herein, the term “heterocyclyl” refers to a saturated or partially unsaturated carbocyclyl group in which one or more ring atoms are heteroatoms independently selected from oxygen, sulfur, nitrogen, phosphorus, and the like, the remaining ring atoms being carbon, wherein one or more ring atoms may be optionally substituted independently with one or more substituents. In some embodiments, the heterocyclyl is a saturated heterocyclyl. In some embodiments, the heterocyclyl is a partially unsaturated heterocyclyl having one or more double bonds in its ring system. In some embodiments, the heterocyclyl may contains any oxidized form of carbon, nitrogen or sulfur, and any quaternized form of a basic nitrogen. The heterocyclyl radical may be carbon linked or nitrogen linked where such is possible. In some embodiments, the heterocycle is carbon linked. In some embodiments, the heterocycle is nitrogen linked. For example, a group derived from pyrrole may be pyrrol-1-yl (nitrogen linked) or pyrrol-3-yl (carbon linked). Further, a group derived from imidazole may be imidazol-1-yl (nitrogen linked) or imidazol-3-yl (carbon linked).

Heterocyclyl group may be monocyclic. Examples of monocyclic heterocyclyl include, but are not limited to oxetanyl, 1,1-dioxothietanylpyrrolidyl, tetrahydrofuryl, tetrahydropyranyl, tetrahydrothienyl, azetidinyl, pyrrolyl, furanyl, thienyl, pyrazolyl, imidazolyl, triazolyl, oxazolyl, thiazolyl, piperidyl, piperazinyl, morpholinyl, pyridinyl, pyrazinyl, pyrimidinyl, pyridazinyl, triazinyl, pyridonyl, pyrimidonyl, pyrazinonyl, pyrimidonyl, pyridazonyl, pyrrolidinyl, triazinonyl, and the like.

Heterocyclyl group may be polycyclic, including the fused, spiro and bridged ring systems. The fused heterocyclyl group includes radicals wherein the heterocyclyl radicals are fused with a saturated, partially unsaturated, or fully unsaturated (i.e., aromatic) carbocyclic or heterocyclic ring. Examples of fused heterocyclyl include, but are not limited to, phenyl fused ring or pyridinyl fused ring, such as quinolinyl, isoquinolinyl, quinoxalinyl, quinolizinyl, quinazolinyl, azaindolizinyl, pteridinyl, chromenyl, isochromenyl, indolyl, isoindolyl, indolizinyl, indazolyl, purinyl, benzofuranyl, isobenzofuranyl, benzimidazolyl, benzothienyl, benzothiazolyl, carbazolyl, phenazinyl, phenothiazinyl, phenanthridinyl, imidazo[1,2-a]pyridinyl, furo[3,4-d]pyrimidinyl, pyrrolo[3,4-d]pyrimidinyl, dihydrofuro[3,4-b]pyridinyl groups, and the like. Examples of spiro heterocyclyl include, but are not limited to, spiropyranyl, spirooxazinyl, 5-aza-spiro[2.4]heptanyl, 6-aza-spiro[2.5]octanyl, 6-aza-spiro[3.4]octanyl, 2-oxa-6-aza-spiro[3.3]heptanyl, 2-oxa-6-aza-spiro[3.4]octanyl, 6-aza-spiro[3.5]nonanyl, 7-aza-spiro[3.5]nonanyl, 1-oxa-7-aza-spiro[3.5]nonanyl, 3,8-dioxa-1-azaspiro[4.5]dec-1-enyl and the like. Examples of bridged heterocyclyl include, but are not limited to, 3-aza-bicyclo[3.1.0]hexanyl, 8-aza-bicyclo[3.2.1]octanyl, 1-aza-bicyclo[2.2.2]octanyl, 2-aza-bicyclo[2.2.1]heptanyl, 1,4-diazabicyclo[2.2.2]octanyl, and the like.

As used herein, the term “heterocyclylalkyl” refers to a heterocyclyl attached to alkyl, including -heterocyclyl-alkyl and heterocyclyl-alkyl-. In some embodiments, heterocyclylalkyl refers to -heterocyclyl-alkyl.

As used herein, the term “hydroxyl” refers to —OH.

“Optional” or “optionally” means that the subsequently described event or circumstance may or may not occur, and that the description includes instances where the said event or circumstance occurs and instances in which it does not.

As used herein, the term “partially unsaturated” refers to a radical that includes at least one double or triple bond. The term “partially unsaturated” is intended to encompass rings having multiple sites of unsaturation, but is not intended to include aromatic (i.e., fully unsaturated) moieties.

As used herein, the term “substituted”, whether preceded by the term “optionally” or not, means that one or more hydrogens of the designated moiety are replaced with a suitable substituent. It will be understood that “substitution” or “substituted with” includes the implicit proviso that such substitution is in accordance with permitted valence of the substituted atom and that the substitution results in a stable or chemically feasible compound, e.g., which does not spontaneously undergo transformation such as by rearrangement, cyclization, elimination, etc. Unless otherwise indicated, an “optionally substituted” group may have a suitable substituent at each substitutable position of the group, and when more than one position in any given structure may be substituted with more than one substituent selected from a specified group, the substituent may be either the same or different at every position. The substituents may include, but not limited to, alkyl, alkenyl, alkynyl, alkoxy, acyl, amino, amido, amidino, aryl, azido, carbamoyl, carboxyl, carboxyl ester, cyano, guanidino, halo, haloalkyl, heteroalkyl, heteroaryl, heterocyclyl, hydroxy, hydrazino, imino, oxo, nitro, alkylsulfinyl, sulfonic acid, alkylsulfonyl, thiocyanate, thiol, thione, or combinations thereof. It will be understood by those skilled in the art that substituents can themselves be substituted, if appropriate. Unless specifically stated as “unsubstituted”, references to chemical moieties herein are understood to include substituted variants. For example, reference to an “aryl” group or moiety implicitly includes both substituted and unsubstituted variants.

As used herein, the term “targeting moiety” refers to a molecule, complex, or aggregate, that binds specifically or selectively to a target molecule, cell, particle, tissue or aggregate. Examples of targeting moiety includes, but are not limited to antibody, antibody binding fragment, bispecific antibody, immunoglobins or other antibody-based molecule or compound. However, other examples of targeting moieties are known in the art and may be used, such as aptamers, avimers, receptor-binding ligands, nucleic acids, biotin-avidin binding pairs, peptides, small molecules, nanoparticles or proteins, etc. The terms “targeting moiety” and “binding moiety” are used synonymously herein.

As used herein, the term “antibody” includes any immunoglobulin, monoclonal antibody, polyclonal antibody, multivalent antibody, multispecific antibody, or bispecific (bivalent) antibody or a functional portion thereof that binds to a specific antigen. A native intact antibody comprises two heavy chains (H) and two light (L) chains inter-connected by disulfide bonds. Each heavy chain consists of a variable region (VH) and a first, second, and third constant region (CH1, CH2 and CH3, respectively), while each light chain consists of a variable region (VL) and a constant region (CL). Mammalian heavy chains are classified as α, δ, ε, γ, and μ, and mammalian light chains are classified as λ or κ. The variable regions of the light and heavy chains are responsible for antigen binding. The variables region in both chains are generally subdivided into three regions of hypervariability called the complementarity determining regions (CDRs) (light (L) chain CDRs including LCDR1, LCDR2, and LCDR3, heavy (H) chain CDRs including HCDR1, HCDR2, HCDR3). CDR boundaries for the antibodies and antigen-binding fragments disclosed herein may be defined or identified by the conventions of Kabat, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol. Biol., 196,901 (1987); Chothia, C. et al., Nature. December 21-28; 342(6252):877-83 (1989); Kabat E. A. et al., National Institutes of Health, Bethesda, Md. (1991)). The three CDRs are interposed between flanking stretches known as framework regions (FRs), which are more highly conserved than the CDRs and form a scaffold to support the hypervariable loops. Therefore, each VH and VL comprises of three CDRs and four FRs in the following order (amino acid residues N terminus to C terminus): FR1, CDR1, FR2, CDR2, FR3, CDR3, FR4. The constant regions of the heavy and light chains are not involved in antigen binding, but exhibit various effector functions. Antibodies are assigned to the five major classes based on the amino acid sequence of the constant region of their heavy chain: IgA, IgD, IgE, IgG, and IgM, which are characterized by the presence of α, δ, ε, γ, and μ heavy chains, respectively. Subclasses of several of the major antibody classes are such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain).

As used herein, the term “antigen-binding fragment” refers to an antibody fragment formed from a fragment of an antibody comprising one or more CDRs, or any other antibody portion that binds to an antigen but does not comprise an intact native antibody structure. Examples of antigen-binding fragment include, but are not limited to, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, an isolated CDR and a bivalent domain antibody.

As used herein, the term “Fab” with regard to an antibody refers to a monovalent antigen-binding fragment of the antibody consisting of a single light chain (both variable and constant regions) bound to the variable region and first constant region of a single heavy chain by a disulfide bond. Fab can be obtained by papain digestion of an antibody at the residues proximal to the N-terminus of the disulfide bond between the heavy chains of the hinge region.

As used herein, the term “Fab′” refers to a Fab fragment that includes a portion of the hinge region, which can be obtained by pepsin digestion of an antibody at the residues proximal to the C-terminus of the disulfide bond between the heavy chains of the hinge region and thus is different from Fab in a small number of residues (including one or more cysteines) in the hinge region.

As used herein, the term “F(ab′)₂” refers to a dimer of Fab′ that comprises two light chains and part of two heavy chains.

As used herein, the term “Fc” with regard to an antibody refers to that portion of the antibody consisting of the second and third constant regions of a first heavy chain bound to the second and third constant regions of a second heavy chain via disulfide bond. IgG and IgM Fc regions contain three heavy chain constant regions (second, third and fourth heavy chain constant regions in each chain). It can be obtained by papain digestion of an antibody. The Fc portion of the antibody is responsible for various effector functions such as ADCC, and CDC, but does not function in antigen binding.

As used herein, the term “Fv” with regard to an antibody refers to the smallest fragment of the antibody to bear the complete antigen binding site. A Fv fragment consists of the variable region of a single light chain bound to the variable region of a single heavy chain. A “dsFv” refers to a disulfide-stabilized Fv fragment that the linkage between the variable region of a single light chain and the variable region of a single heavy chain is a disulfide bond.

As used herein, the term “single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85:5879(1988)). A “scFv dimer” refers to a single chain comprising two heavy chain variable regions and two light chain variable regions with a linker. An “scFv dimer” may be a bivalent diabody or bivalent ScFv (BsFv) comprising V_{H}-V_{L} (linked by a peptide linker) dimerized with another V_{H}-V_{L} moiety such that V_{H}'S of one moiety coordinate with the V_{L}'s of the other moiety and form two binding sites which can target the same antigens (or eptipoes) or different antigens (or eptipoes). A “scFv dimer” may also be a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate and each coordinated pair has a different antigen specificity.

As used herein, the term “single-chain Fv-Fc antibody” or “scFv-Fc” refers to an engineered antibody consisting of a scFv connected to the Fc region of an antibody.

As used herein, the term “camelized single domain antibody,” “heavy chain antibody,” “nanobody” or “HCAb” refers to an antibody that contains two V_{H} domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally obtained from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. “Heavy-chain antibodies in Camelidae; a case of evolutionary innovation,” Immunogenetics. April; 54(1):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)). “Diabodies” include small antibody fragments with two antigen-binding sites, wherein the fragments comprise a V_{H} domain connected to a V_{L} domain in a single polypeptide chain (V_{H}-V_{L} or V_{L}-V_{H}) (see, e.g., Holliger P. et al., Proc Natl Acad Sci USA. July 15; 90(14):6444-8 (1993); EP404097; WO93/11161). The two domains on the same chain cannot be paired, because the linker is too short, thus, the domains are forced to pair with the complementary domains of another chain, thereby creating two antigen-binding sites. The antigen-binding sites may target the same of different antigens (or epitopes).

As used herein, the term “domain antibody” refers to an antibody fragment containing only the variable region of a heavy chain or the variable region of a light chain. In some embodiments, two or more V_{H} domains are covalently joined with a peptide linker to form a bivalent or multivalent domain antibody. The two V_{H} domains of a bivalent domain antibody may target the same or different antigens.

As used herein, the term “(dsFv)₂” refers to an antigen binding fragment consisting of three peptide chains: two V_{H} moieties linked by a peptide linker and bound by disulfide bridges to two V_{L} moieties.

As used herein, the term “bispecific ds diabody” refers to an antigen binding fragment consisting of V_{H1}-V_{L2} (linked by a peptide linker) bound to V_{L1}-V_{H2} (also linked by a peptide linker) via a disulfide bridge between V_{H1} and V_{L1}.

As used herein, the term “bispecific dsFv” or “dsFv-dsFv′” refers to a antigen binding fragment consisting of three peptide chains: a V_{H1}-V_{H2} moiety wherein the heavy chains are bound by a peptide linker (e.g., a long flexible linker) and paired via disulfide bridges to V_{L1} and V_{L2} moieties, respectively. Each disulfide paired heavy and light chain has a different antigen specificity.

In some embodiment, the antibody or its antigen binding fragment is chimeric or humanized.

As used herein, the term “chimeric” refers to an antibody or antigen-binding fragment that has a portion of heavy and/or light chain derived from one species, and the rest of the heavy and/or light chain derived from a different species. In an illustrative example, a chimeric antibody may comprise a constant region derived from human and a variable region derived from a non-human species, such as from mouse.

As used herein, the term “cell-interacting molecule” refers to a molecule that is capable of interacting with a cell surface material of a target cell to trigger or promote a conjugate compound containing such cell-interacting molecules to specifically bind to a cell, to trigger or promote endocytosis of the conjugate compound by a target cell, and/or to trigger or promote the conjugate compound to be enriched around a target cell and/or enter a target cell. The cell-interacting molecule may be a small chemical molecule, a linear or macrocyclic peptide or a large biomolecule. For example, the cell-interacting molecule is, but not limited to, a small molecule compound, or a polypeptide comprising 2-50, 2-40, 2-30, 2-25, 2-22, 2-20, 2-18, 2-15, 2-12, 2-10, 2-8, 4-50, 5-50, 5-40, 5-30, 5-25, 5-22, 5-20, 5-18, 5-15, 5-12, 5-10, 6, 7, 8, or 9 amino acids.

As used herein, the term “humanized”, with reference to antibody or antigen-binding fragment, refers to the antibody or the antigen-binding fragment comprises CDRs derived from non-human animals (e.g. a rodent, rabbit, dog, goat, horse, or chicken), FR regions derived from human, and when applicable, the constant regions derived from human. In some embodiments, the constant regions from a human antibody are fused to the non-human variable regions. A humanized antibody or antigen-binding fragment is useful as human therapeutics. In some embodiments because it has reduced immunogenicity or is less likely to induce an immune response in human, as compared to the non-human species antibody. In some embodiments, the non-human animal is a mammal, for example, a mouse, a rat, a rabbit, a goat, a sheep, a guinea pig, a hamster, or a non-human primate (for example, a monkey (e.g., cynomolgus or rhesus monkey) or an ape (e.g., chimpanzee, gorilla, simian or affen)). In some embodiments, the humanized antibody or antigen-binding fragment is composed of substantially all human sequences except for the CDR sequences which are non-human. In some embodiments, the humanized antibody or antigen-binding fragment is modified to improve the antibody performance, such as binding or binding affinity. For example, one or more amino acid residues in one or more non-human CDRs are altered to reduce potential immunogenicity in human, wherein the altered amino acid residues either are not critical for immunospecific binding or the alterations are conservative changes, such that the binding of the humanized antibody to the antigen is not significantly affected. In some embodiments, the FR regions derived from human may comprise the same amino acid sequence as the human antibody from which it is derived, or it may comprise some amino acid changes, for example, no more than 10, 9, 8, 7, 6, 5, 4, 3, 2, or 1 changes of amino acid. In some embodiments, such change in amino acid could be present in heavy chain FR regions only, in light chain FR regions only, or in both chains. In some preferable embodiments, the humanized antibodies comprise human FR1-3 and human JH and Jκ.

As used herein, the term “ligand” refers to a variety of chemical or biological molecules, which can have a specific binding affinity to a selected target, wherein the selected target can be, for example, a cell surface receptor, a cell surface antigen, a cell, a tissue, an organ, etc. In some embodiments, the ligand can specifically bind to a protein or a marker expressed on the surface of a target cell. In some embodiments, the ligand of the present disclosure binds to a cell surface protein or marker with an affinity of 10⁻⁶-10⁻¹¹ M (K_{d} value). In some embodiments, the ligand of the present disclosure binds to a cell surface protein or marker with an affinity of at least 10⁻⁷, at least 10⁻⁸ and at least 10⁻⁹ M (K_{d} value). In some embodiments, the ligand of the present disclosure binds to a cell surface protein or marker with an affinity of less than 10⁻⁶, less than 10⁻⁷ and less than 10⁻⁸ M (K_{d} value). In some embodiments, the ligand of the present disclosure binds to a cell surface protein or marker with a certain affinity, wherein the certain affinity refers to the affinity of the ligand to a target cell surface protein or marker which is at least two, three, four, five, six, eight, ten, twenty, fifty, one hundred or more times higher than that to a non-target cell surface protein or marker. In some embodiments, the expression of the cell surface protein or marker of the present disclosure in target cells (e.g. cancer cells) is significantly higher than that in normal cells. The term “significantly” as used herein refers to statistically significant differences, or significant differences that can be recognized by a person skilled in the art.

In some embodiments, the expression level of the cell surface protein or marker of the present disclosure in target cells (e.g. cancer cells) are 2 to 1,000,000 times higher than that in normal cells; for example, the expression level in target cells (e.g. cancer cells) are 2 to 10, 2 to 100, 2 to 1,000, 2 to 10,000, 2 to 100,000 or 2 to 1,000,000 (which can be equal to any value within the above numerical range, and the end values of this range included) times higher than that in normal cells. In some embodiments, the expression level of the cell surface receptor in target cells (e.g. cancer cells) is at least 10 times higher, or 100 times higher, or 1,000 times higher, or 10,000 times higher, or 100,000 times higher than that in normal cells. In some embodiments, compared with the level of the cell surface protein or marker on target cells (e.g. cancer cells), the level of the cell surface receptor on normal cells is reduced by at least 50%, 60%, 70%, 80%, 90%, 95%, or 99%. In some embodiments, the cell surface protein or marker described in the present disclosure is undetectable in normal cells.

As used herein, the term “nanoparticle” refers to any particle having a diameter of less than 1000 nm. For example, the nanoparticle has a diameter of, but not limited to, 1-990 nm, 10-950 nm, 10-900 nm, 10-800 nm, 10-700 nm, 10-600 nm, 10-500 nm, 10-400 nm, 10-300 nm, 10-200 nm, 10-100 nm, 50-900 nm, 100-800 nm, 200-700 nm, 300-600 nm, or 400-500 nm.

As used herein, the term “nucleic acid” refers to any polynucleotide that binds to a component associated with an organ, tissue, cell, extracellular matrix component, and/or intracellular compartment (the target). In some embodiments, nucleic acid targeting moieties are aptamers. Aptamers are polynucleotide molecules that have been selected (e.g., from random or mutagenized pools) on the basis of their ability to bind to another molecule. In some embodiments, the aptamer comprises a DNA polynucleotide. In some embodiments, the aptamer comprises an RNA polynucleotide. In some embodiments, the aptamer comprises one or more modified nucleic acid residues. Methods of generating and screening nucleic acid aptamers for binding to proteins are well known in the art. See, e.g., U.S. Pat. Nos. 5,683,867, 6,344,321, 7,329,742, and International Patent Publication No. WO 03/070984, each of which is incorporated by reference herein in its entirety.

As used herein, the term “polypeptide”, “protein” or “peptide” can be a single amino acid or a polymer of amino acids. The polypeptide, protein or peptide as described in the present disclosure may contain naturally-occurring amino acids and non-naturally-occurring amino acids, or analogs and mimetics thereof. The polypeptide, protein or peptide can be obtained by any method well known in the art, for example, but not limited to, by an isolation and a purification from natural materials, a recombinant expression, a chemical synthesis, etc.

As used herein, the term “small molecule” refers to a compound having a molecular weight of less than or equal to about 2 kDa. For example, the small molecule compound has a molecular weight of less than or equal to, but not limited to, about 1.5 kDa, 1 kDa, 800 Da, 700 Da, 600 Da, or 500 Da.

As used herein, the term “specific binding” or “specifically binds” refers to a non-random binding reaction between two molecules, such as for example between an antibody and an antigen. In some embodiments, the antibodies or antigen-binding fragments provided herein specifically bind to a target antigen with a binding affinity (K_{D}) of about 0.01 nM to about 100 nM, about 0.1 nM to about 100 nM, 0.01 nM to about 10 nM, about 0.1 nM to about 10 nM, 0.01 nM to about 1 nM, about 0.1 nM to about 1 nM or about 0.01 nM to about 0.1 nM) at pH 7.4. K_{D} as used herein refers to the ratio of the dissociation rate to the association rate (k_{off}/kₒₙ), may be determined using surface plasmon resonance methods for example using instrument such as Biacore.

As used herein, the term “tumor antigen” refers to an antigenic substance produced in tumor cells, i.e., it triggers an immune response in the host. Normal proteins in the body are not antigenic because of self-tolerance, a process in which self-reacting cytotoxic T lymphocytes (CTLs) and autoantibody -producing B lymphocytes are culled “centrally” in primary lymphatic tissue (BM) and “peripherally” in secondary lymphatic tissue (mostly thymus for T-cells and spleen/lymph nodes for B cells). Thus, any protein that is not exposed to the immune system triggers an immune response. This may include normal proteins that are well sequestered from the immune system, proteins that are normally produced in extremely small quantities, proteins that are normally produced only in certain stages of development, or proteins whose structure is modified due to mutation.

### Payload Compound

In one aspect, there is provided a payload compound having Formula (II′):

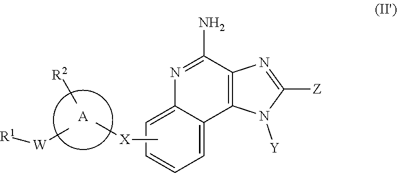

or a pharmaceutically acceptable salt thereof, wherein:
   X is selected from the group consisting of —O—, —S—, —NH—, —(CH₂)ᵢ—, —(X¹)NC(O)—, —(X¹)NS(O)₂—, —C(O)N(X¹)— and —S(O)₂N(X¹)—, wherein —NH— and —(CH₂)ᵢ— are optionally substituted with one or more groups independently selected from halogen, hydroxyl, cyano, amino or alkyl;
   each X¹ is independently hydrogen, alkyl, alkenyl, or haloalkyl;
   Ring A is cycloalkyl, heteroalkyl, aryl or heteroaryl;
   W is selected from the group consisting of a direct bond, alkyl, cycloalkyl, heterocyclyl, *—W¹-alkyl-, *-alkyl-W¹—, and *-alkyl-W¹-alkyl-, wherein the alkyl, cycloalkyl, heterocyclyl, and alkyl in *—W¹-alkyl-, *-alkyl-W¹—, and *-alkyl-W¹-alkyl are optionally substituted with one or more groups independently selected from halogen, hydroxyl, cyano, amino, alkyl and —C(O)OR^{a}, and wherein * end of W is connected to Ring A;
   W¹ is —O—, —NR^{a}—, —C(O)—, —C(O)NR^{a}—, —OC(O)NR^{a}—, —NR^{a}C(O)—, —NR^{a}C(O)O—, or —NR^{a}C(O)NR^{a}—,
   each R^{a} is independently hydrogen, alkyl, or haloalkyl;
   R¹ is hydrogen, —N(R^{b})₂, hydroxyl, or —SH;
      each R^{b} is independently hydrogen, alkyl, or haloalkyl, or
      two R^{b} taken together with the nitrogen atom to which they are bound form a heterocyclyl;
   R² is hydrogen, halogen, hydroxyl, cyano, amino, alkyl, alkenyl, alkynyl, alkoxyl, haloalkyl, —OR^{e}, —OC(O)R^{e}, —SR^{e}, —B(OH)₂, —NO₂, —CHO, —C(═NOR^{e})(R^{e}), —R²⁻¹—N(R^{e})₂, —R²⁻¹—N(R^{e})C(O)R^{e}, —R²⁻¹—N(R^{e})S(O)₂R^{e}, —R²⁻¹—N(R^{e})P(O)₂R^{e}, —R²⁻¹—C(O)OR^{e}, —R²⁻¹—C(O)N(R^{e})₂, —R²⁻¹—S(O)₂N(R^{e})₂, —R²⁻¹—P(O)₂N(R^{e})₂, —OC(O)NR^{e}, or —NC(O)NR^{e},
      each R²⁻¹ is independently null or alkyl;
      each R^{e} is independently hydrogen, alkyl, or haloalkyl;
   Y is —Y¹—Y²—Y³, wherein
      Y¹ is a direct bond or —(CH₂)ₘ-Q¹-(CH₂)ₙ—O—*, wherein —(CH₂)ₘ— and —(CH₂)ₙ— are optionally substituted with one or more groups independently selected from halogen, hydroxyl, alkyl or haloalkyl, and * end of Y¹ is connected to Y²;
      Y² is a direct bond or —(CH₂)ₛ-Q²-(CH₂)ₜ—NR^{c}—**, wherein —(CH₂)ₛ— and —(CH₂)ₜ— are optionally substituted with one or more groups independently selected from halogen, hydroxyl, alkyl or haloalkyl, and ** end of Y² is connect to Y³;
      Y³ is selected from the group consisting of hydrogen, alkyl, -alkyl-aryl, -alkyl-N(R^{b})₂, —C(O)-alkyl, —C(O)-alkyl-N(R^{b})₂, and —S(O)₂-alkyl;
      Q¹ and Q² are each independently selected from a direct bond, cycloalkyl, heterocyclyl, aryl, or heteroaryl, wherein the cycloalkyl, heterocyclyl, aryl, and heteroaryl are optionally substituted with one or more groups independently selected from halogen, hydroxyl, cyano, amino, or alkyl;
      R^{c} is hydrogen or alkyl, or
      R^{c} and Y³ taken together with the atoms to which they are bound form a heterocyclyl optionally substituted with one or more groups independently selected from halogen, hydroxyl, cyano, amino or alkyl;
   Z is selected from the group consisting of alkyl, alkenyl, alkynyl, heteroalkyl, heteroalkenyl and heteroalkynyl, each of which is optionally substituted with one or more R^{d};
      R^{d} is selected from the group consisting of halogen, acyl, alkyl, alkenyl, alkynyl, cycloalkyl and —O-cycloalkyl;
   i is 0, 1, 2, 3, 4, 5 or 6;
   m is 0, 1, 2, 3, 4 or 5;
   n is 0, 1, 2, 3, 4 or 5;
   s is 0, 1, 2, 3, 4 or 5; and
   t is 0, 1, 2, 3, 4 or 5.

In some embodiments, ring A is selected from the group consisting of:

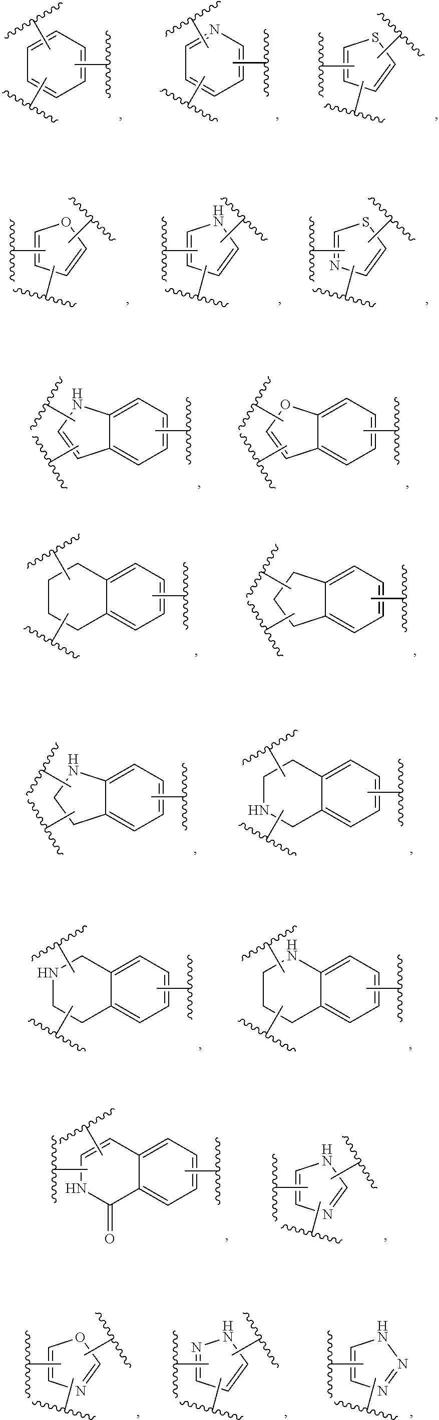

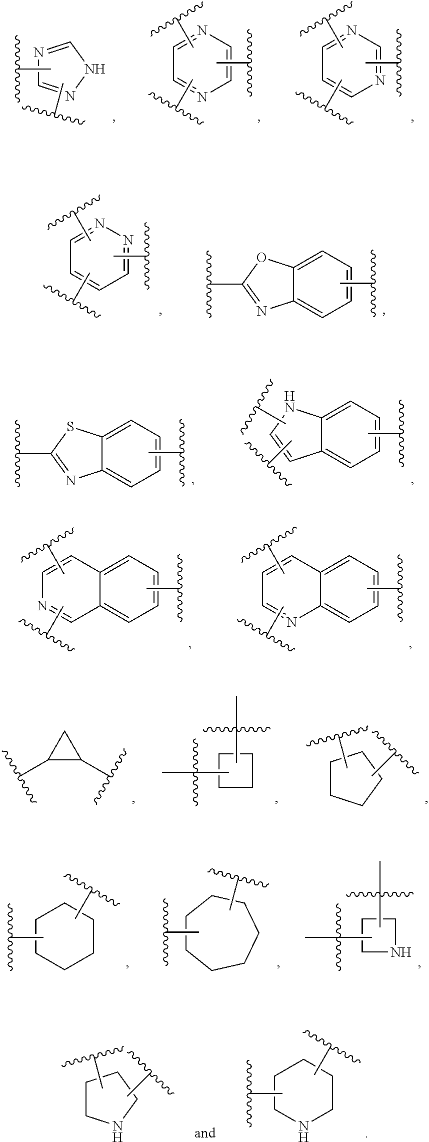

In some embodiments, X is —O—, —NH—, or —(CH₂)ᵢ—, wherein —NH— and —(CH₂)ᵢ— are optionally substituted with one or more halogen or alkyl.

In certain embodiments, X is —O—, —N(CH₃)—, —CH₂—, —(CH₂)₂—, —(CH₂)₃—, —(CH₂)₄—, —C(CH₃)₂— or —CF₂—.

In some embodiments, R¹ is hydrogen, —N(R^{b})₂ or hydroxyl.

In some embodiments, W is a direct bond.

In certain embodiments, W is a direct bond, R¹ is hydrogen, —N(R^{b})₂ or hydroxyl, and each R^{b} is independently hydrogen or alkyl, or two R^{b} taken together with the nitrogen atom to which they are bound form a heterocyclyl.

In some embodiments, W is alkyl optionally substituted with —C(O)OR^{a}. In certain embodiments, W is C₁₋₆ alkyl, C₁₋₅ alkyl, C₁₋₄ alkyl, C₁₋₃ alkyl, or C₁₋₂ alkyl, optionally substituted with —C(O)OR^{a}. In certain embodiments, W is methyl, ethyl or propyl, each optionally substituted with —C(O)OR^{a}. In certain embodiments, R^{a} is C₁₋₆ alkyl, C₁₋₅ alkyl, C₁₋₄ alkyl, C₁₋₃ alkyl, or C₁₋₂ alkyl.

In certain embodiments, W is alkyl optionally substituted with —C(O)OR^{a}, R¹ is hydrogen, hydroxyl or —N(R^{b})₂, and each R^{b} is independently hydrogen or alkyl, or two R^{b} taken together with the nitrogen atom to which they are bound form a heterocyclyl. In certain embodiments, R¹ is hydrogen. In certain embodiments, R¹ is hydroxyl. In certain embodiments, R¹ is —N(R^{b})₂, and each R^{b} is independently hydrogen, C₁₋₆ alkyl, C₁₋₅ alkyl, C₁₋₄ alkyl, C₁₋₃ alkyl, or C₁₋₂ alkyl. In certain embodiments, R¹ is —N(R^{b})₂, and each R^{b} is independently hydrogen or methyl. In certain embodiments, R¹ is —N(R^{b})₂, and two R^{b} taken together with the nitrogen atom to which they are bound form a 3 to 10 membered heterocyclyl, 3 to 9 membered heterocyclyl, 3 to 8 membered heterocyclyl, 3 to 7 membered heterocyclyl, 3 to 6 membered heterocyclyl, or 3 to 5 membered heterocyclyl.

In some embodiments, W is *—W¹-alkyl-. In certain embodiments, W is *—W¹—C₁₋₆ alkyl-, *—W¹—C₁₋₅ alkyl-, *—W¹—C₁₋₄ alkyl-, *—W¹—C₁₋₃ alkyl-, or *—W¹—C₁₋₂ alkyl-.

In certain embodiments, W is *—W¹-alkyl-, W¹ is —O—, —NR^{a}—, —C(O)NR^{a}—, —OC(O)NR^{a}—, —NR^{a}C(O)—, —NR^{a}C(O)O—, or —NR^{a}C(O)NR^{a}—, and each R^{a} is independently hydrogen, alkyl, or haloalkyl.

In certain embodiments, W is *—W¹-alkyl-, and R¹ is —NH₂ or —NH—CH₃.

In some embodiments, W is *-alkyl-W¹—. In certain embodiments, W is *—C₁₋₆ alkyl-W¹—, *—C₁₋₅ alkyl-W¹—, *—C₁₋₄ alkyl-W¹—, *—C₁₋₃ alkyl-W¹—, or *—C₁₋₂ alkyl-W¹—.

In certain embodiments, W is *-alkyl-W¹—, and W¹ is —C(O)—.

In certain embodiments, W is *-alkyl-W¹—, W¹ is —C(O)—, and R¹ is —NH₂.

In some embodiments, W is *-alkyl-W¹-alkyl-. In certain embodiments, W is *-alkyl-W¹-alkyl-, and each alkyl in W is independently C₁₋₆ alkyl, C₁₋₅ alkyl, C₁₋₄ alkyl, C₁₋₃ alkyl, or C₁₋₂ alkyl.

In certain embodiments, W is *-alkyl-W¹-alkyl-, W¹ is —NR^{a}C(O)— or —OC(O)NR^{a}—, and R^{a} is hydrogen, alkyl, or haloalkyl.

In certain embodiments, W is *-alkyl-W¹-alkyl-, W¹ is —NR^{a}C(O)—, and R¹ is —NH₂.

In certain embodiments, W is *-alkyl-W¹-alkyl-, W¹ is —NR^{a}C(O)—, and R¹ is hydroxyl.

In certain embodiments, W is *-alkyl-W¹-alkyl-, W¹ is —OC(O)NR^{a}—, and R¹ is —NH—CH₃.

In some embodiments, W is cycloalkyl. In certain embodiments, W is C₃₋₁₀ cycloalkyl, C₃₋₉ cycloalkyl, C₃₋₅ cycloalkyl, C₃₋₇ cycloalkyl, C₃₋₆ cycloalkyl, or C₃₋₅ cycloalkyl.

In certain embodiments, W is cycloalkyl, and R¹ is —NH₂.

In some embodiment, W is heterocyclyl. In certain embodiments, W is 3 to 10 membered heterocyclyl, 3 to 9 membered heterocyclyl, 3 to 8 membered heterocyclyl, 3 to 7 membered heterocyclyl, 3 to 6 membered heterocyclyl, or 3 to 5 membered heterocyclyl.

In certain embodiments, W is heterocyclyl, and R¹ is hydrogen.

In some embodiments, R² is hydrogen, halogen, cyano, alkyl or alkoxyl. In certain embodiments, R² is hydrogen.

In some embodiments, Y¹ is a direct bond.

In certain embodiments, Y¹ is a direct bond, and Y² is a direct bond.

In certain embodiments, Y¹ is a direct bond, Y² is a direct bond, and Y³ is -alkyl-aryl.

In certain embodiments, Y¹ is a direct bond, and Y² is —(CH₂)ₛ-Q²-(CH₂)ₜ—NR^{c}—**. In certain embodiments, Q² is a direct bond, cycloalkyl or aryl, wherein the cycloalkyl or aryl is optionally substituted with one or more groups independently selected from halogen or alkyl. In certain embodiments, R¹ is hydrogen.

In certain embodiments, Y¹ is a direct bond, Y² is —(CH₂)ₛ-Q²-(CH₂)ₜ—NR^{c}—**, and Y³ is hydrogen, -alkyl-NH₂, or —S(O)₂-alkyl. In certain embodiments, the alkyl in -alkyl-NH₂ and —S(O)₂-alkyl is independently C₁₋₆ alkyl, C₁₋₅ alkyl, C₁₋₄ alkyl, C₁₋₃ alkyl, or C₁₋₂ alkyl.

In some embodiments, Y¹ is —(CH₂)ₘ-Q¹-(CH₂)ₙ—O—*, wherein —(CH₂)ₘ— and —(CH₂)ₙ— are optionally substituted with one or more groups independently selected from halogen, hydroxyl, alkyl or haloalkyl. In certain embodiments, —(CH₂)ₘ— and —(CH₂)ₙ— are optionally substituted with one or more groups independently selected from alkyl or haloalkyl. In certain embodiments, —(CH₂)ₘ— and —(CH₂)ₙ— are optionally substituted with one or more C₁₋₆ alkyl or C₁₋₆ haloalkyl. In certain embodiments, —(CH₂)ₘ— and —(CH₂)ₙ— are optionally substituted with one or more groups independently selected from methyl, ethyl or trifluoromethyl. In certain embodiments, Q¹ is a direct bond, cycloalkyl or aryl, wherein the cycloalkyl or aryl is optionally substituted with one or more groups independently selected from halogen or alkyl.

In certain embodiments, Y¹ is —(CH₂)ₘ-Q¹-(CH₂)ₙ—O—*, wherein —(CH₂)ₘ— and —(CH₂)ₙ— are optionally substituted with one or more groups independently selected from halogen, hydroxyl, alkyl or haloalkyl, and Y² is a direct bond. In certain embodiments, —(CH₂)ₘ— and —(CH₂)ₙ— are optionally substituted with one or more groups independently selected from alkyl or haloalkyl. In certain embodiments, —(CH₂)ₘ— and —(CH₂)ₙ— are optionally substituted with one or more C₁₋₆ alkyl or C₁₋₆ haloalkyl. In certain embodiments, —(CH₂)ₘ— and —(CH₂)ₙ— are optionally substituted with one or more groups independently selected from methyl, ethyl or trifluoromethyl.

In certain embodiments, Y¹ is —(CH₂)ₘ-Q¹-(CH₂)ₙ—O—*, Y² is a direct bond, and Y³ is hydrogen. In certain embodiments, —(CH₂)ₘ— and —(CH₂)ₙ— are optionally substituted with one or more groups independently selected from alkyl or haloalkyl. In certain embodiments, —(CH₂)ₘ— and —(CH₂)ₙ— are optionally substituted with one or more C₁₋₆ alkyl or C₁₋₆ haloalkyl. In certain embodiments, —(CH₂)ₘ— and —(CH₂)ₙ— are optionally substituted with one or more groups independently selected from methyl, ethyl or trifluoromethyl.

In certain embodiments, Y¹ is —(CH₂)ₘ-Q¹-(CH₂)ₙ—O—*, and Y² is —(CH₂)ₛ-Q²-(CH₂)ₜ—NR^{c}—**. In certain embodiments, Q² is a direct bond. In certain embodiments, R^{c} is hydrogen. In certain embodiments, —(CH₂)ₘ— and —(CH₂)ₙ— are optionally substituted with one or more groups independently selected from alkyl or haloalkyl. In certain embodiments, —(CH₂)ₘ— and —(CH₂)ₙ— are optionally substituted with one or more C₁₋₆ alkyl or C₁₋₆ haloalkyl. In certain embodiments, —(CH₂)ₘ— and —(CH₂)ₙ— are optionally substituted with one or more groups independently selected from methyl, ethyl or trifluoromethyl.

In certain embodiments, Y¹ is —(CH₂)ₘ-Q¹-(CH₂)ₙ—O—*, Y² is —(CH₂)ₛ-Q²-(CH₂)ₜ—NR^{c}—**, and Y³ is hydrogen, —C(O)-alkyl or —C(O)-alkyl-N(R^{b})₂. In certain embodiments, —(CH₂)ₘ— and —(CH₂)ₙ— are optionally substituted with one or more groups independently selected from alkyl or haloalkyl. In certain embodiments, —(CH₂)ₘ— and —(CH₂)ₙ— are optionally substituted with one or more C₁₋₆ alkyl or C₁₋₆ haloalkyl. In certain embodiments, —(CH₂)ₘ— and —(CH₂)ₙ— are optionally substituted with one or more groups independently selected from methyl, ethyl or trifluoromethyl. In certain embodiments, R^{c} and Y³ taken together with the atoms to which they are bound form a heterocyclyl optionally substituted with one or more halogen.

In some embodiments, Z is selected from the group consisting of alkyl, alkenyl, alkynyl and heteroalkyl, which is optionally substituted with one or more R^{d}. In certain embodiments, Z is selected from the group consisting of C₁₋₆ alkyl, C₁₋₆ alkenyl, C₁₋₆ alkynyl and C₁₋₆ heteroalkyl, which is optionally substituted with one or more R^{d}.

In certain embodiments, R^{d} is selected from the group consisting of halogen, acyl, alkyl, cycloalkyl and —O-cycloalkyl. In certain embodiments, R^{d} is selected from the group consisting of halogen, acyl, C₁₋₆ alkyl, C₃₋₆ cycloalkyl and —O—C₃₋₆ cycloalkyl.

In some embodiments, the present disclosure provides a payload compound having Formula (IIa′):

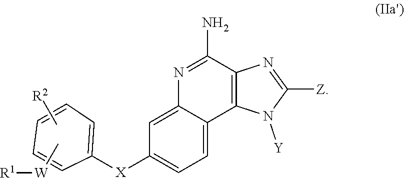

In another aspect, the present disclosure provides a payload compound having a formula selected from the group consisting of:

| Compound # | Compound |
|---|---|
| 1. | 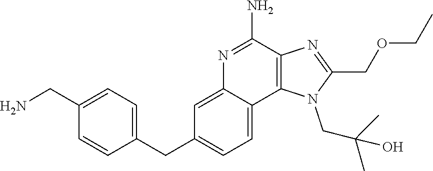 [SMILES: CCOCc1nc2c(N)nc3cc(Cc4ccc(CN)cc4)ccc3c2n1CC(C)(C)O] |
| 2. | 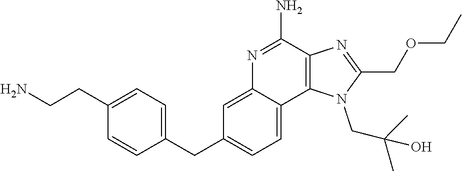 [SMILES: CCOCc1nc2c(N)nc3cc(Cc4ccc(CCN)cc4)ccc3c2n1CC(C)(C)O] |
| 3. | 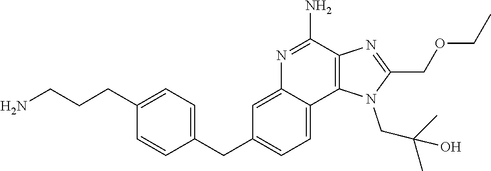 [SMILES: CCOCc1nc2c(N)nc3cc(Cc4ccc(CCCN)cc4)ccc3c2n1CC(C)(C)O] |
| 4. | 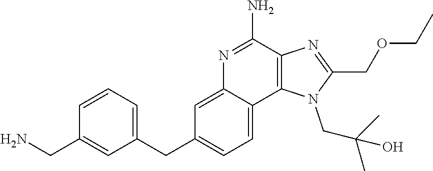 [SMILES: CCOCc1nc2c(N)nc3cc(Cc4cccc(CN)c4)ccc3c2n1CC(C)(C)O] |
| 5. | 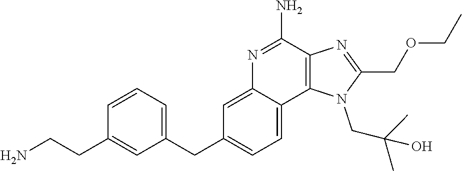 [SMILES: CCOCc1nc2c(N)nc3cc(Cc4cccc(CCN)c4)ccc3c2n1CC(C)(C)O] |
| 6. | 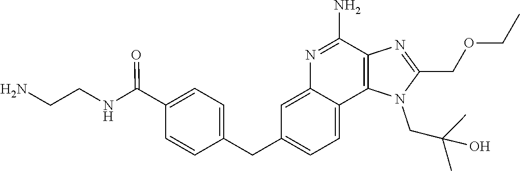 [SMILES: CCOCc1nc2c(N)nc3cc(Cc4ccc(C(=O)NCCN)cc4)ccc3c2n1CC(C)(C)O] |
| 7. | 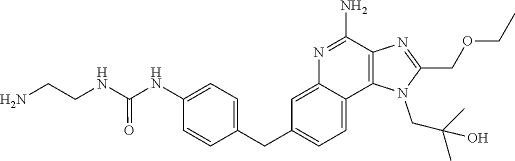 [SMILES: CCOCc1nc2c(N)nc3cc(Cc4ccc(NC(=O)NCCN)cc4)ccc3c2n1CC(C)(C)O] |
| 8. | 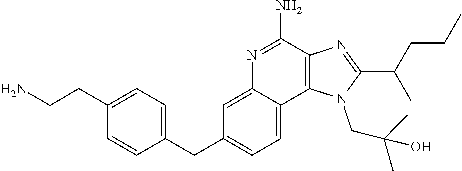 [SMILES: CCCC(C)c1nc2c(N)nc3cc(Cc4ccc(CCN)cc4)ccc3c2n1CC(C)(C)O] |
| 9. | 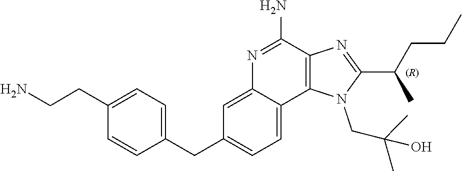 |
| 10. | 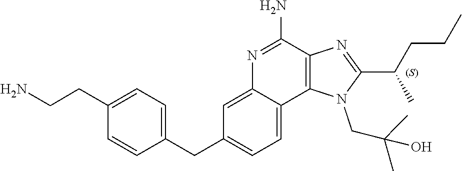 |
| 11. | 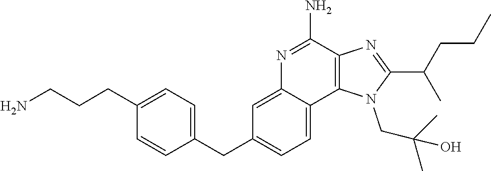 [SMILES: CCCC(C)c1nc2c(N)nc3cc(Cc4ccc(CCCN)cc4)ccc3c2n1CC(C)(C)O] |
| 12. | 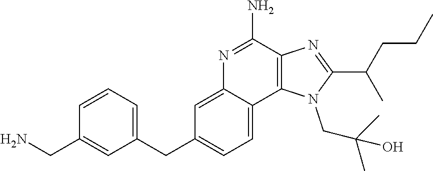 [SMILES: CCCC(C)c1nc2c(N)nc3cc(Cc4cccc(CN)c4)ccc3c2n1CC(C)(C)O] |
| 13. | 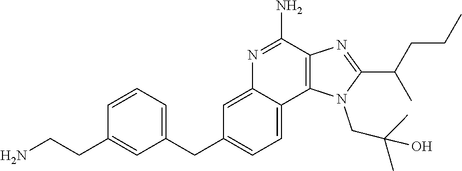 [SMILES: CCCC(C)c1nc2c(N)nc3cc(Cc4cccc(CCN)c4)ccc3c2n1CC(C)(C)O] |
| 14. | 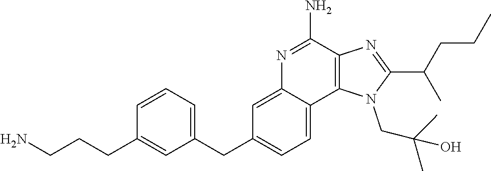 [SMILES: CCCC(C)c1nc2c(N)nc3cc(Cc4cccc(CCCN)c4)ccc3c2n1CC(C)(C)O] |
| 15. | 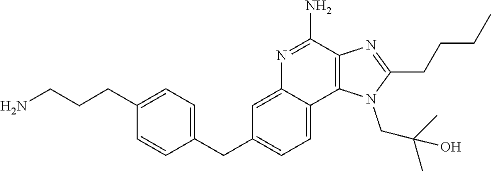 [SMILES: CCCCc1nc2c(N)nc3cc(Cc4ccc(CCCN)cc4)ccc3c2n1CC(C)(C)O] |
| 16. | 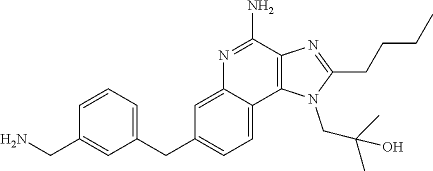 [SMILES: CCCCc1nc2c(N)nc3cc(Cc4cccc(CN)c4)ccc3c2n1CC(C)(C)O] |
| 17. | 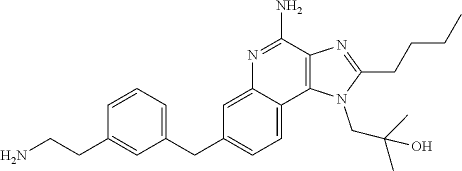 [SMILES: CCCCc1nc2c(N)nc3cc(Cc4cccc(CCN)c4)ccc3c2n1CC(C)(C)O] |
| 18. | 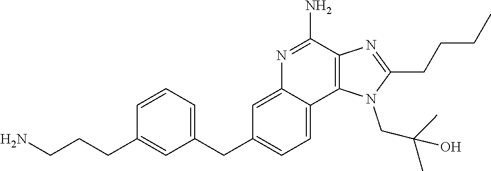 [SMILES: CCCCc1nc2c(N)nc3cc(Cc4cccc(CCCN)c4)ccc3c2n1CC(C)(C)O] |
| 19. | 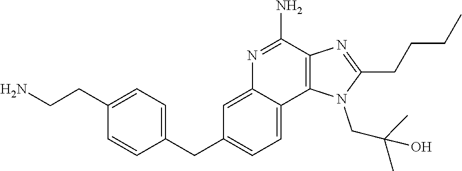 [SMILES: CCCCc1nc2c(N)nc3cc(Cc4ccc(CCN)cc4)ccc3c2n1CC(C)(C)O] |
| 20. | 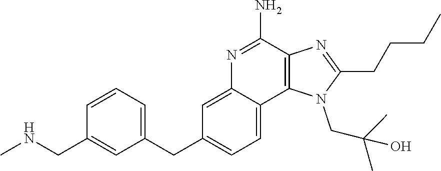 [SMILES: CCCCc1nc2c(N)nc3cc(Cc4cccc(CNC)c4)ccc3c2n1CC(C)(C)O] |
| 21. | 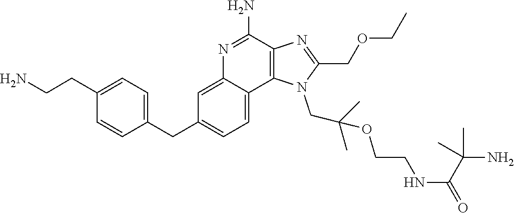 [SMILES: CCOCc1nc2c(N)nc3cc(Cc4ccc(CCN)cc4)ccc3c2n1CC(C)(C)OCCNC(=O)C(C)(C)N] |
| 22. | 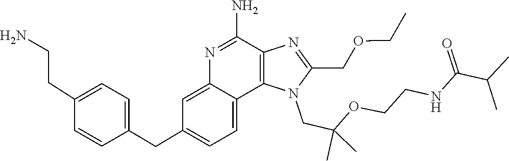 [SMILES: CCOCc1nc2c(N)nc3cc(Cc4ccc(CCN)cc4)ccc3c2n1CC(C)(C)OCCNC(=O)C(C)C] |
| 23. | 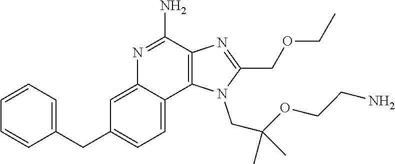 [SMILES: CCOCc1nc2c(N)nc3cc(Cc4ccccc4)ccc3c2n1CC(C)(C)OCCN] |
| 24. | 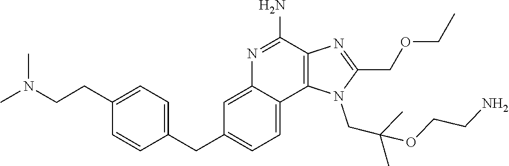 [SMILES: CCOCc1nc2c(N)nc3cc(Cc4ccc(CCN(C)C)cc4)ccc3c2n1CC(C)(C)OCCN] |
| 25. | 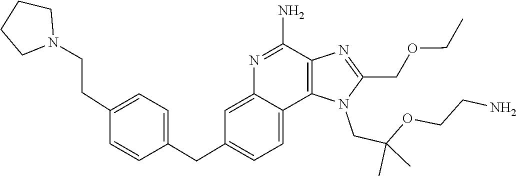 [SMILES: CCOCc1nc2c(N)nc3cc(Cc4ccc(CCN5CCCC5)cc4)ccc3c2n1CC(C)(C)OCCN] |
| 26. | 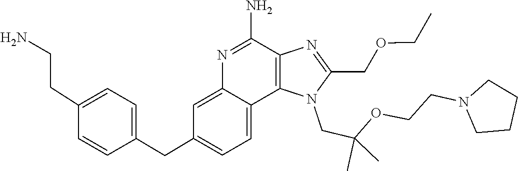 [SMILES: CCOCc1nc2c(N)nc3cc(Cc4ccc(CCN)cc4)ccc3c2n1CC(C)(C)OCCN1CCCC1] |
| 27. | 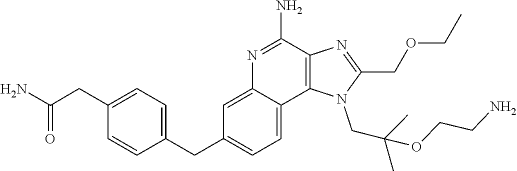 [SMILES: CCOCc1nc2c(N)nc3cc(Cc4ccc(CC(N)=O)cc4)ccc3c2n1CC(C)(C)OCCN] |
| 28. | 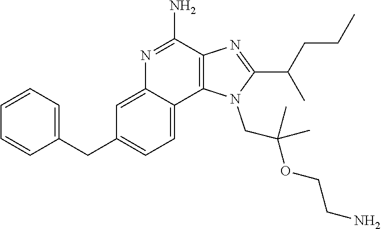 [SMILES: CCCC(C)c1nc2c(N)nc3cc(Cc4ccccc4)ccc3c2n1CC(C)(C)OCCN] |
| 29. | 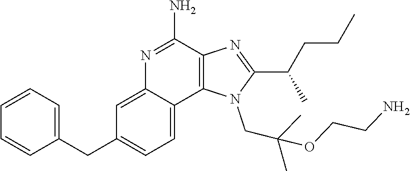 [SMILES: CCC[C@H](C)c1nc2c(N)nc3cc(Cc4ccccc4)ccc3c2n1CC(C)(C)OCCN] |
| 30. | 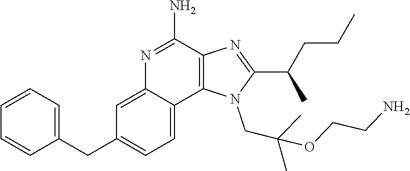 [SMILES: CCC[C@@H](C)c1nc2c(N)nc3cc(Cc4ccccc4)ccc3c2n1CC(C)(C)OCCN] |
| 31. | 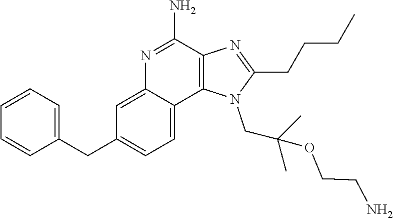 [SMILES: CCCCc1nc2c(N)nc3cc(Cc4ccccc4)ccc3c2n1CC(C)(C)OCCN] |
| 32. | 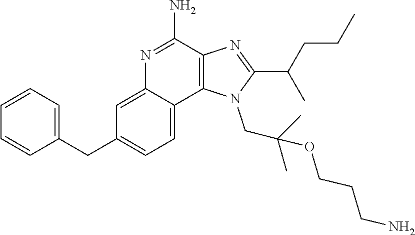 [SMILES: CCCC(C)c1nc2c(N)nc3cc(Cc4ccccc4)ccc3c2n1CC(C)(C)OCCCN] |
| 33. | 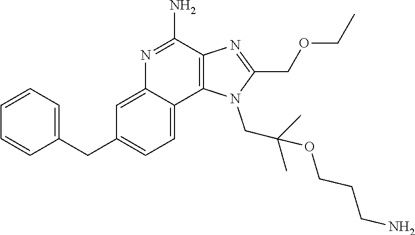 [SMILES: CCOCc1nc2c(N)nc3cc(Cc4ccccc4)ccc3c2n1CC(C)(C)OCCCN] |
| 34. | 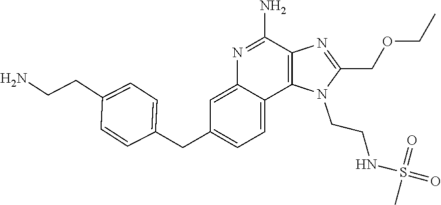 [SMILES: CCOCc1nc2c(N)nc3cc(Cc4ccc(CCN)cc4)ccc3c2n1CCNS(C)(=O)=O] |
| 35. | 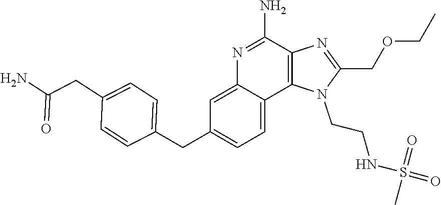 [SMILES: CCOCc1nc2c(N)nc3cc(Cc4ccc(CC(N)=O)cc4)ccc3c2n1CCNS(C)(=O)=O] |
| 36. | 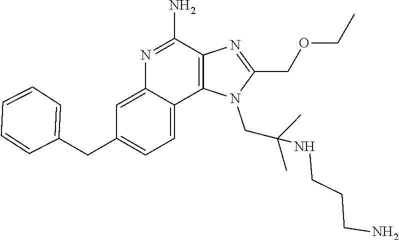 [SMILES: CCOCc1nc2c(N)nc3cc(Cc4ccccc4)ccc3c2n1CC(C)(C)NCCCN] |
| 37. | 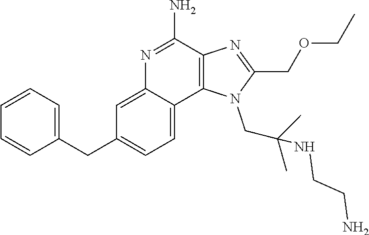 [SMILES: CCOCc1nc2c(N)nc3cc(Cc4ccccc4)ccc3c2n1CC(C)(C)NCCN] |
| 38. | 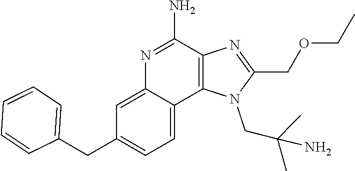 [SMILES: CCOCc1nc2c(N)nc3cc(Cc4ccccc4)ccc3c2n1CC(C)(C)N] |
| 39. | 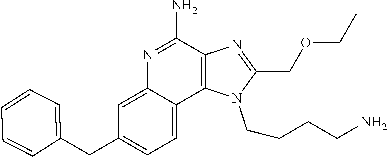 [SMILES: CCOCc1nc2c(N)nc3cc(Cc4ccccc4)ccc3c2n1CCCCN] |
| 40. | 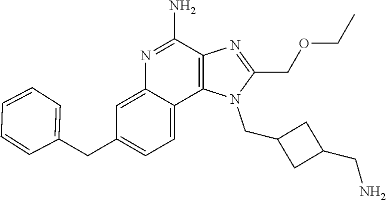 [SMILES: CCOCc1nc2c(N)nc3cc(Cc4ccccc4)ccc3c2n1CC1CC(CN)C1] |
| 41. | 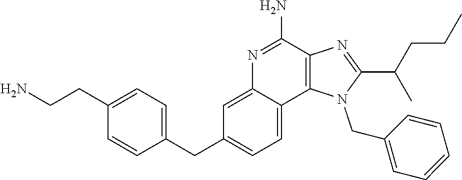 [SMILES: CCCC(C)c1nc2c(N)nc3cc(Cc4ccc(CCN)cc4)ccc3c2n1Cc1ccccc1] |
| 42. | 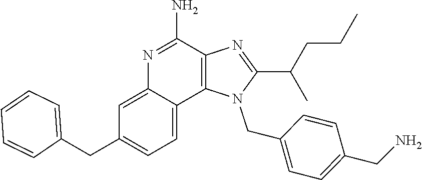 [SMILES: CCCC(C)c1nc2c(N)nc3cc(Cc4ccccc4)ccc3c2n1Cc1ccc(CN)cc1] |
| 43. | 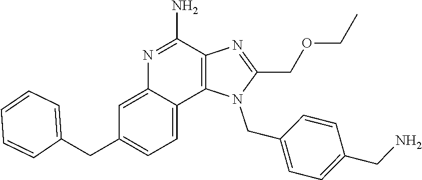 [SMILES: CCOCc1nc2c(N)nc3cc(Cc4ccccc4)ccc3c2n1Cc1ccc(CN)cc1] |
| 44. | 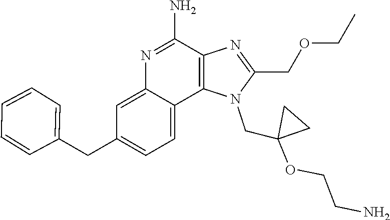 [SMILES: CCOCc1nc2c(N)nc3cc(Cc4ccccc4)ccc3c2n1CC1(OCCN)CC1] |
| 45. | 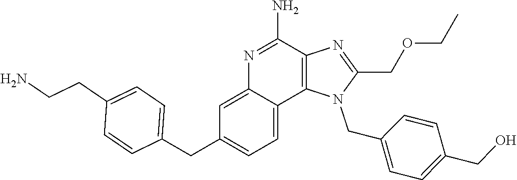 [SMILES: CCOCc1nc2c(N)nc3cc(Cc4ccc(CCN)cc4)ccc3c2n1Cc1ccc(CO)cc1] |
| 46. | 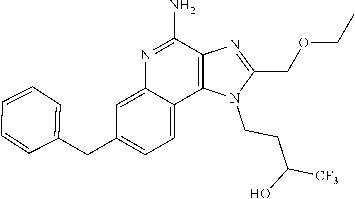 [SMILES: CCOCc1nc2c(N)nc3cc(Cc4ccccc4)ccc3c2n1CCC(O)C(F)(F)F] |
| 47. | 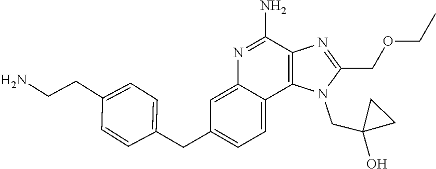 [SMILES: CCOCc1nc2c(N)nc3cc(Cc4ccc(CCN)cc4)ccc3c2n1CC1(O)CC1] |
| 48. | 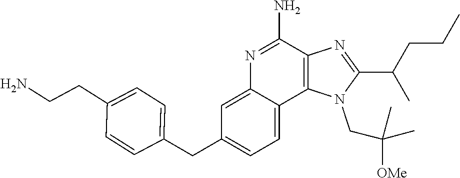 [SMILES: CCCC(C)c1nc2c(N)nc3cc(Cc4ccc(CCN)cc4)ccc3c2n1CC(C)(C)OC] |
| 49. | 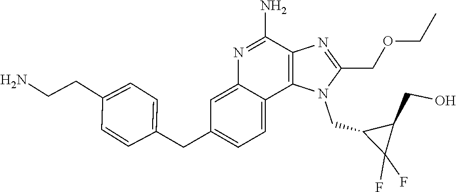 [SMILES: CCOCc1nc2c(N)nc3cc(Cc4ccc(CCN)cc4)ccc3c2n1C[C@@H]1[C@@H](CO)C1(F)F] |
| 50. | 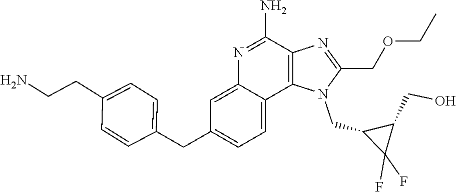 [SMILES: CCOCc1nc2c(N)nc3cc(Cc4ccc(CCN)cc4)ccc3c2n1C[C@@H]1[C@H](CO)C1(F)F] |
| 51. | 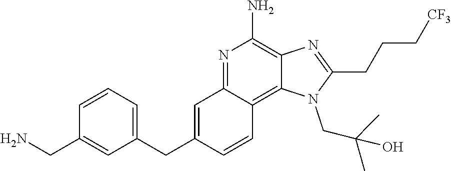 [SMILES: CC(C)(O)Cn1c(CCCC(F)(F)F)nc2c(N)nc3cc(Cc4cccc(CN)c4)ccc3c21] |
| 52. | 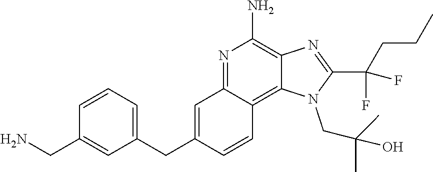 [SMILES: CCCC(F)(F)c1nc2c(N)nc3cc(Cc4cccc(CN)c4)ccc3c2n1CC(C)(C)O] |
| 53. | 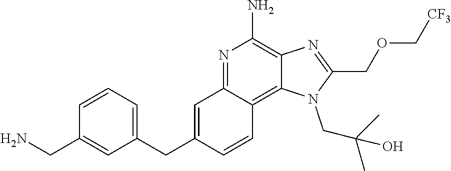 [SMILES: CC(C)(O)Cn1c(COCC(F)(F)F)nc2c(N)nc3cc(Cc4cccc(CN)c4)ccc3c21] |
| 54. | 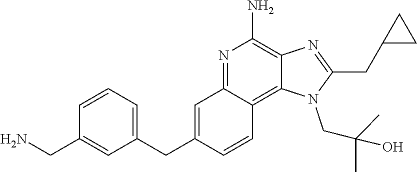 [SMILES: CC(C)(O)Cn1c(CC2CC2)nc2c(N)nc3cc(Cc4cccc(CN)c4)ccc3c21] |
| 55. | 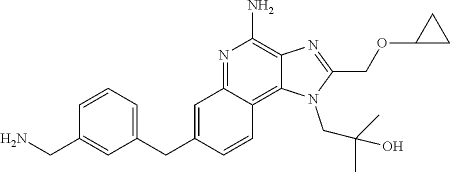 [SMILES: CC(C)(O)Cn1c(COC2CC2)nc2c(N)nc3cc(Cc4cccc(CN)c4)ccc3c21] |
| 56. | 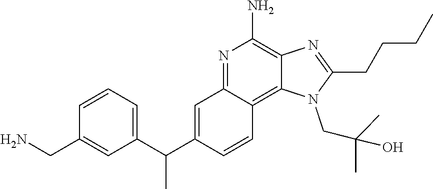 [SMILES: CCCCc1nc2c(N)nc3cc(C(C)c4cccc(CN)c4)ccc3c2n1CC(C)(C)O] |
| 57. | 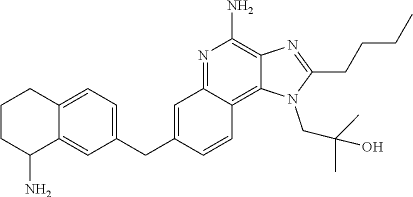 [SMILES: CCCCc1nc2c(N)nc3cc(Cc4ccc5c(c4)C(N)CCC5)ccc3c2n1CC(C)(C)O] |
| 58. | 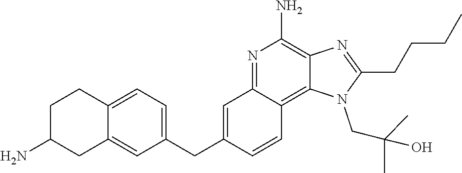 [SMILES: CCCCc1nc2c(N)nc3cc(Cc4ccc5c(c4)CC(N)CC5)ccc3c2n1CC(C)(C)O] |
| 59. | 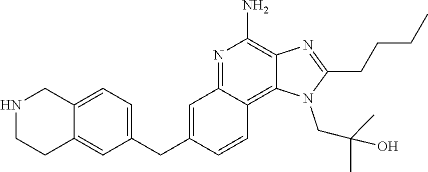 [SMILES: CCCCc1nc2c(N)nc3cc(Cc4ccc5c(c4)CCNC5)ccc3c2n1CC(C)(C)O] |
| 60. | 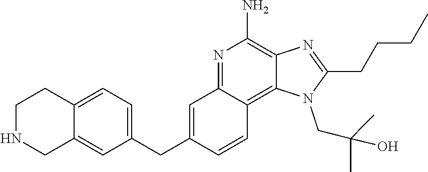 [SMILES: CCCCc1nc2c(N)nc3cc(Cc4ccc5c(c4)CNCC5)ccc3c2n1CC(C)(C)O] |
| 61. | 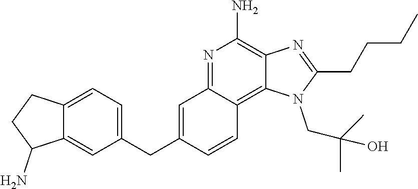 [SMILES: CCCCc1nc2c(N)nc3cc(Cc4ccc5c(c4)C(N)CC5)ccc3c2n1CC(C)(C)O] |
| 62. | 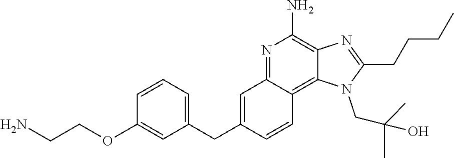 [SMILES: CCCCc1nc2c(N)nc3cc(Cc4cccc(OCCN)c4)ccc3c2n1CC(C)(C)O] |
| 63. | 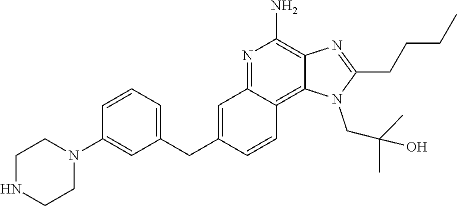 [SMILES: CCCCc1nc2c(N)nc3cc(Cc4cccc(N5CCNCC5)c4)ccc3c2n1CC(C)(C)O] |
| 64. | 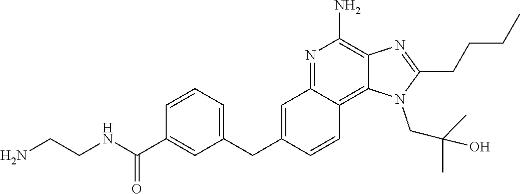 [SMILES: CCCCc1nc2c(N)nc3cc(Cc4cccc(C(=O)NCCN)c4)ccc3c2n1CC(C)(C)O] |
| 65. | 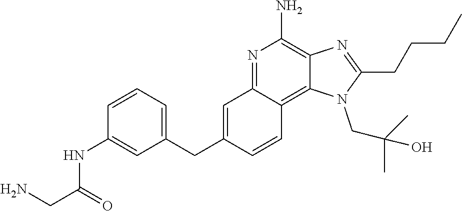 [SMILES: CCCCc1nc2c(N)nc3cc(Cc4cccc(NC(=O)CN)c4)ccc3c2n1CC(C)(C)O] |
| 66. | 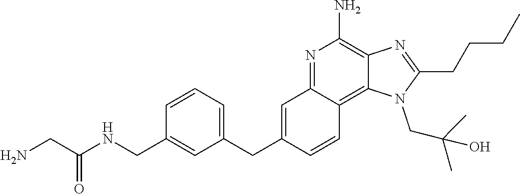 [SMILES: CCCCc1nc2c(N)nc3cc(Cc4cccc(CNC(=O)CN)c4)ccc3c2n1CC(C)(C)O] |
| 67. | 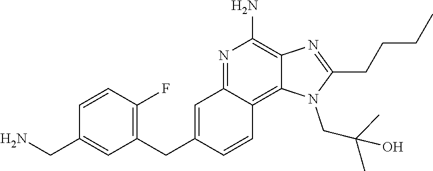 [SMILES: CCCCc1nc2c(N)nc3cc(Cc4cc(CN)ccc4F)ccc3c2n1CC(C)(C)O] |
| 68. | 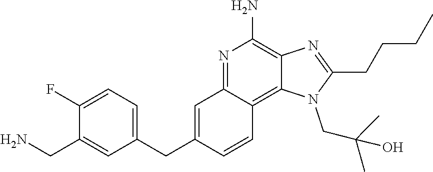 [SMILES: CCCCc1nc2c(N)nc3cc(Cc4ccc(F)c(CN)c4)ccc3c2n1CC(C)(C)O] |
| 69. | 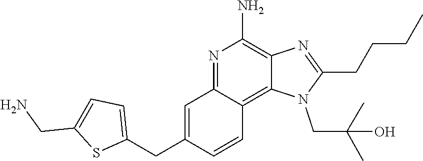 [SMILES: CCCCc1nc2c(N)nc3cc(Cc4ccc(CN)s4)ccc3c2n1CC(C)(C)O] |
| 70. | 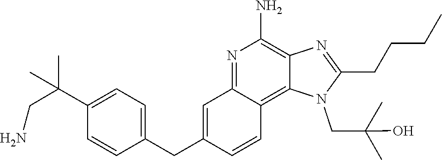 [SMILES: CCCCc1nc2c(N)nc3cc(Cc4ccc(C(C)(C)CN)cc4)ccc3c2n1CC(C)(C)O] |
| 71. | 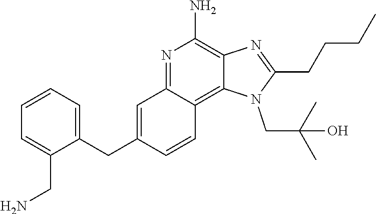 [SMILES: CCCCc1nc2c(N)nc3cc(Cc4ccccc4CN)ccc3c2n1CC(C)(C)O] |
| 72. | 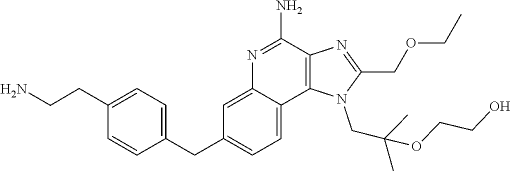 [SMILES: CCOCc1nc2c(N)nc3cc(Cc4ccc(CCN)cc4)ccc3c2n1CC(C)(C)OCCO] |
| 73. | 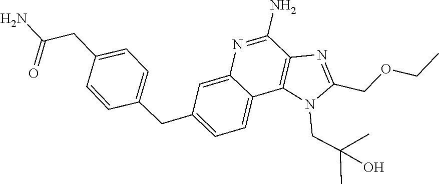 [SMILES: CCOCc1nc2c(N)nc3cc(Cc4ccc(CC(N)=O)cc4)ccc3c2n1CC(C)(C)O] |
| 74. | 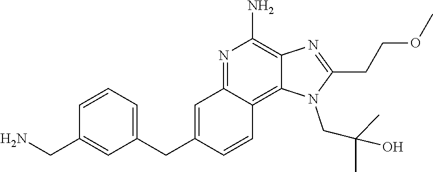 [SMILES: COCCc1nc2c(N)nc3cc(Cc4cccc(CN)c4)ccc3c2n1CC(C)(C)O] |
| 75. | 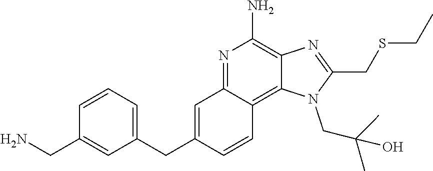 [SMILES: CCSCc1nc2c(N)nc3cc(Cc4cccc(CN)c4)ccc3c2n1CC(C)(C)O] |
| 76. | 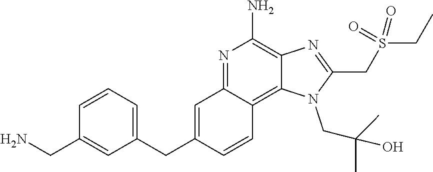 [SMILES: CCS(=O)(=O)Cc1nc2c(N)nc3cc(Cc4cccc(CN)c4)ccc3c2n1CC(C)(C)O] |
| 77. | 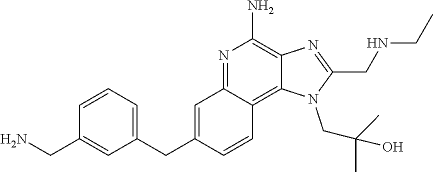 [SMILES: CCNCc1nc2c(N)nc3cc(Cc4cccc(CN)c4)ccc3c2n1CC(C)(C)O] |
| 78. | 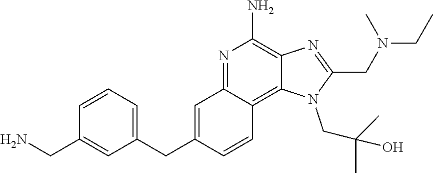 [SMILES: CCN(C)Cc1nc2c(N)nc3cc(Cc4cccc(CN)c4)ccc3c2n1CC(C)(C)O] |
| 79. | 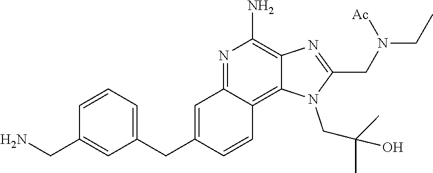 [SMILES: CCN(Cc1nc2c(N)nc3cc(Cc4cccc(CN)c4)ccc3c2n1CC(C)(C)O)C(C)=O] |
| 80. | 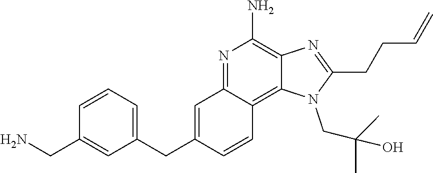 [SMILES: C=CCCc1nc2c(N)nc3cc(Cc4cccc(CN)c4)ccc3c2n1CC(C)(C)O] |
| 81. | 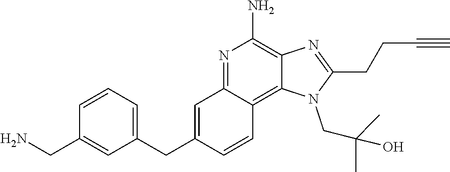 [SMILES: C#CCCc1nc2c(N)nc3cc(Cc4cccc(CN)c4)ccc3c2n1CC(C)(C)O] |
| 82. | 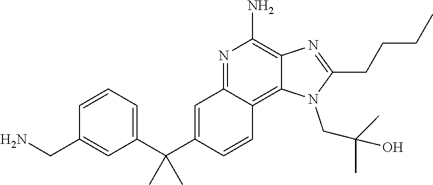 [SMILES: CCCCc1nc2c(N)nc3cc(C(C)(C)c4cccc(CN)c4)ccc3c2n1CC(C)(C)O] |
| 83. | 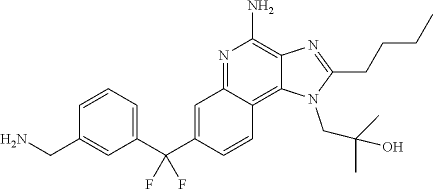 [SMILES: CCCCc1nc2c(N)nc3cc(C(F)(F)c4cccc(CN)c4)ccc3c2n1CC(C)(C)O] |
| 84. | 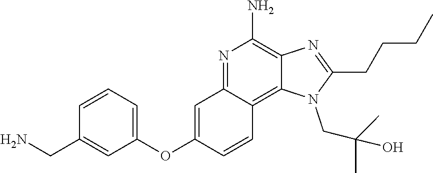 [SMILES: CCCCc1nc2c(N)nc3cc(Oc4cccc(CN)c4)ccc3c2n1CC(C)(C)O] |
| 85. | 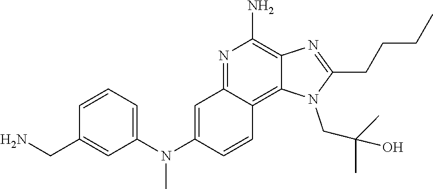 [SMILES: CCCCc1nc2c(N)nc3cc(N(C)c4cccc(CN)c4)ccc3c2n1CC(C)(C)O] |
| 86. | 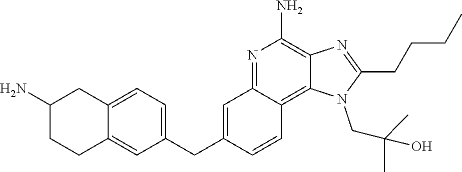 [SMILES: CCCCc1nc2c(N)nc3cc(Cc4ccc5c(c4)CCC(N)C5)ccc3c2n1CC(C)(C)O] |
| 87. | 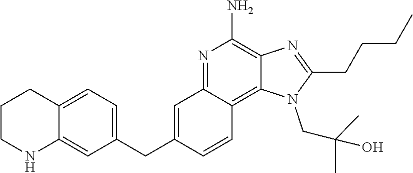 [SMILES: CCCCc1nc2c(N)nc3cc(Cc4ccc5c(c4)NCCC5)ccc3c2n1CC(C)(C)O] |
| 88. | 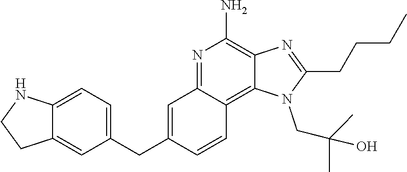 [SMILES: CCCCc1nc2c(N)nc3cc(Cc4ccc5c(c4)CCN5)ccc3c2n1CC(C)(C)O] |
| 89. | 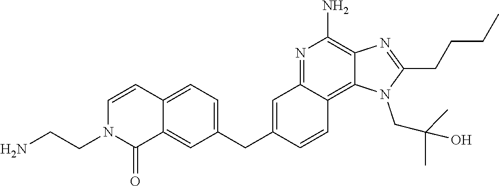 [SMILES: CCCCc1nc2c(N)nc3cc(Cc4ccc5ccn(CCN)c(=O)c5c4)ccc3c2n1CC(C)(C)O] |
| 90. | 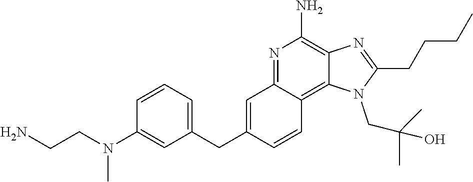 [SMILES: CCCCc1nc2c(N)nc3cc(Cc4cccc(N(C)CCN)c4)ccc3c2n1CC(C)(C)O] |
| 91. | 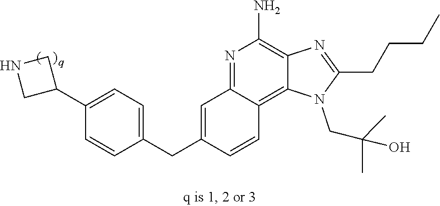 |
| 92. | 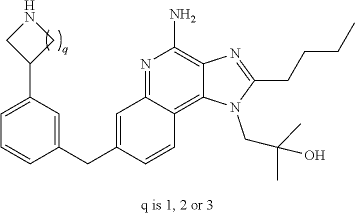 |
| 93. | 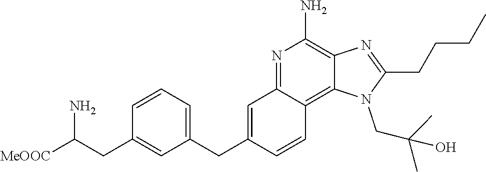 [SMILES: CCCCc1nc2c(N)nc3cc(Cc4cccc(CC(N)C(=O)OC)c4)ccc3c2n1CC(C)(C)O] |
| 94. | 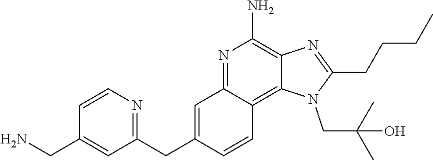 [SMILES: CCCCc1nc2c(N)nc3cc(Cc4cc(CN)ccn4)ccc3c2n1CC(C)(C)O] |
| 95. | 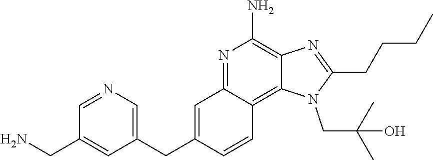 [SMILES: CCCCc1nc2c(N)nc3cc(Cc4cncc(CN)c4)ccc3c2n1CC(C)(C)O] |
| 96. | 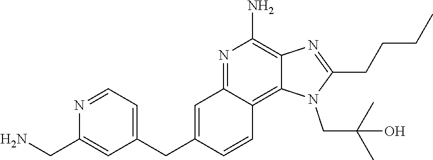 [SMILES: CCCCc1nc2c(N)nc3cc(Cc4ccnc(CN)c4)ccc3c2n1CC(C)(C)O] |
| 97. | 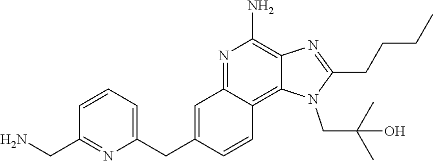 [SMILES: CCCCc1nc2c(N)nc3cc(Cc4cccc(CN)n4)ccc3c2n1CC(C)(C)O] |
| 98. | 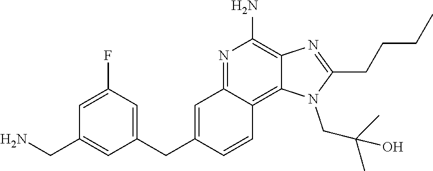 [SMILES: CCCCc1nc2c(N)nc3cc(Cc4cc(F)cc(CN)c4)ccc3c2n1CC(C)(C)O] |
| 99. | 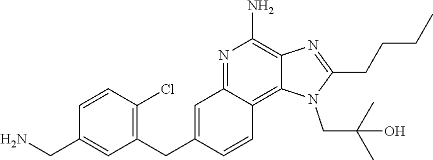 [SMILES: CCCCc1nc2c(N)nc3cc(Cc4cc(CN)ccc4Cl)ccc3c2n1CC(C)(C)O] |
| 100. | 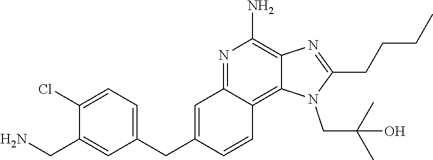 [SMILES: CCCCc1nc2c(N)nc3cc(Cc4ccc(Cl)c(CN)c4)ccc3c2n1CC(C)(C)O] |
| 101. | 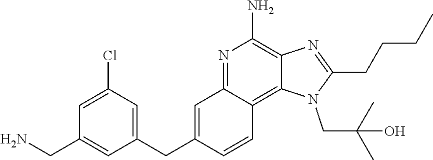 [SMILES: CCCCc1nc2c(N)nc3cc(Cc4cc(Cl)cc(CN)c4)ccc3c2n1CC(C)(C)O] |
| 102. | 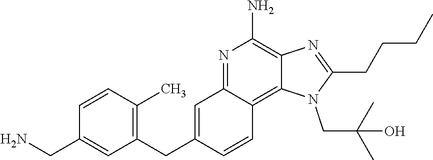 [SMILES: CCCCc1nc2c(N)nc3cc(Cc4cc(CN)ccc4C)ccc3c2n1CC(C)(C)O] |
| 103. | 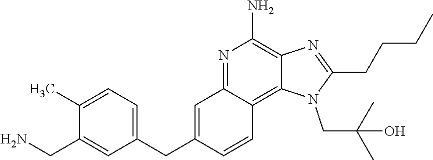 [SMILES: CCCCc1nc2c(N)nc3cc(Cc4ccc(C)c(CN)c4)ccc3c2n1CC(C)(C)O] |
| 104. | 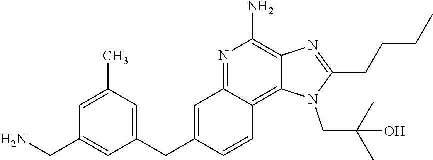 [SMILES: CCCCc1nc2c(N)nc3cc(Cc4cc(C)cc(CN)c4)ccc3c2n1CC(C)(C)O] |
| 105. | 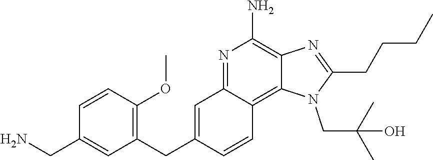 [SMILES: CCCCc1nc2c(N)nc3cc(Cc4cc(CN)ccc4OC)ccc3c2n1CC(C)(C)O] |
| 106. | 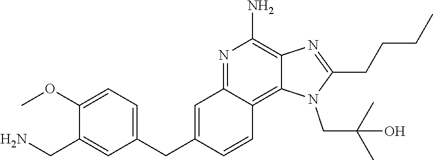 [SMILES: CCCCc1nc2c(N)nc3cc(Cc4ccc(OC)c(CN)c4)ccc3c2n1CC(C)(C)O] |
| 107. | 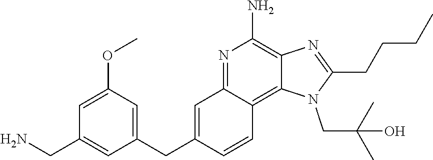 [SMILES: CCCCc1nc2c(N)nc3cc(Cc4cc(CN)cc(OC)c4)ccc3c2n1CC(C)(C)O] |
| 108. | 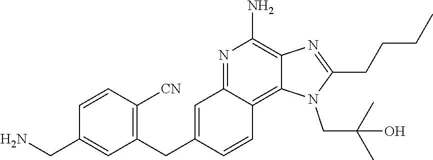 [SMILES: CCCCc1nc2c(N)nc3cc(Cc4cc(CN)ccc4C#N)ccc3c2n1CC(C)(C)O] |
| 109. | 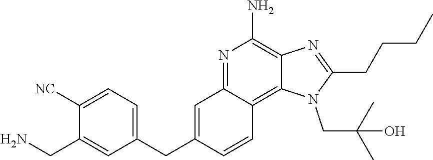 [SMILES: CCCCc1nc2c(N)nc3cc(Cc4ccc(C#N)c(CN)c4)ccc3c2n1CC(C)(C)O] |
| 110. | 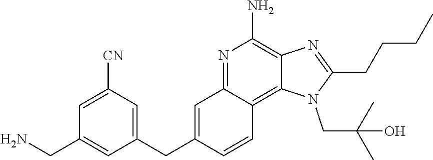 [SMILES: CCCCc1nc2c(N)nc3cc(Cc4cc(C#N)cc(CN)c4)ccc3c2n1CC(C)(C)O] |
| 111. | 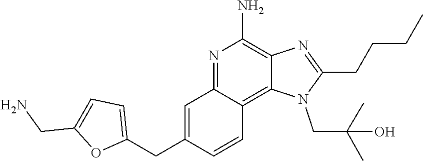 [SMILES: CCCCc1nc2c(N)nc3cc(Cc4ccc(CN)o4)ccc3c2n1CC(C)(C)O] |
| 112. | 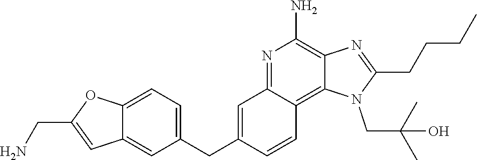 [SMILES: CCCCc1nc2c(N)nc3cc(Cc4ccc5oc(CN)cc5c4)ccc3c2n1CC(C)(C)O] |
| 113. | 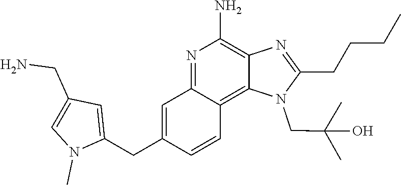 [SMILES: CCCCc1nc2c(N)nc3cc(Cc4cc(CN)cn4C)ccc3c2n1CC(C)(C)O] |
| 114. | 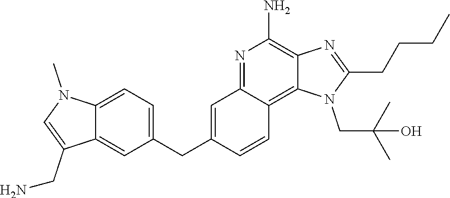 [SMILES: CCCCc1nc2c(N)nc3cc(Cc4ccc5c(c4)c(CN)cn5C)ccc3c2n1CC(C)(C)O] |
| 115. | 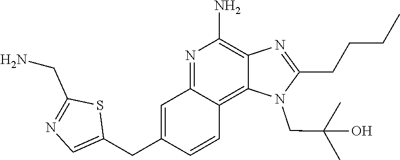 [SMILES: CCCCc1nc2c(N)nc3cc(Cc4cnc(CN)s4)ccc3c2n1CC(C)(C)O] |
| 116. | 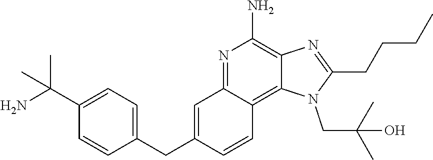 [SMILES: CCCCc1nc2c(N)nc3cc(Cc4ccc(C(C)(C)N)cc4)ccc3c2n1CC(C)(C)O] |
| 117. | 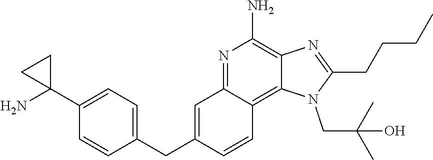 [SMILES: CCCCc1nc2c(N)nc3cc(Cc4ccc(C5(N)CC5)cc4)ccc3c2n1CC(C)(C)O] |
| 118. | 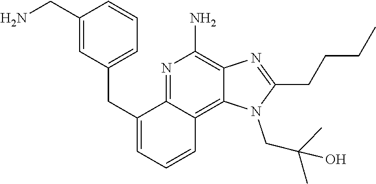 [SMILES: CCCCc1nc2c(N)nc3c(Cc4cccc(CN)c4)cccc3c2n1CC(C)(C)O] |
| 119. | 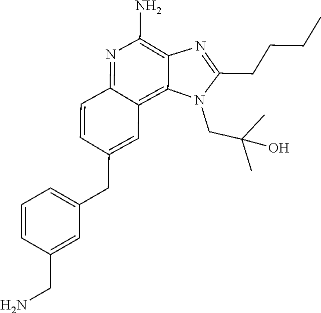 [SMILES: CCCCc1nc2c(N)nc3ccc(Cc4cccc(CN)c4)cc3c2n1CC(C)(C)O] |
| 120. | 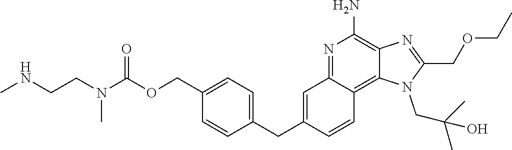 [SMILES: CCOCc1nc2c(N)nc3cc(Cc4ccc(COC(=O)N(C)CCNC)cc4)ccc3c2n1CC(C)(C)O] |
| 121. | 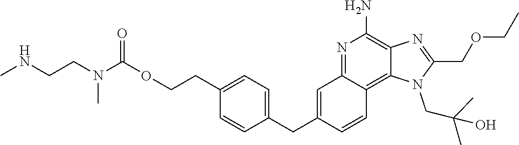 [SMILES: CCOCc1nc2c(N)nc3cc(Cc4ccc(CCOC(=O)N(C)CCNC)cc4)ccc3c2n1CC(C)(C)O] |
| 122. | 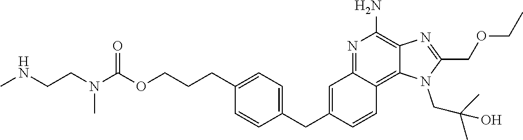 [SMILES: CCOCc1nc2c(N)nc3cc(Cc4ccc(CCCOC(=O)N(C)CCNC)cc4)ccc3c2n1CC(C)(C)O] |
| 123. | 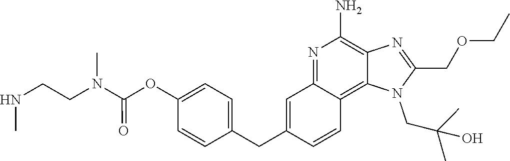 [SMILES: CCOCc1nc2c(N)nc3cc(Cc4ccc(OC(=O)N(C)CCNC)cc4)ccc3c2n1CC(C)(C)O] |
| 124. | 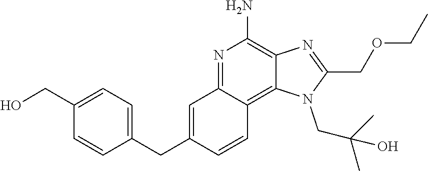 [SMILES: CCOCc1nc2c(N)nc3cc(Cc4ccc(CO)cc4)ccc3c2n1CC(C)(C)O] |
| 125. | 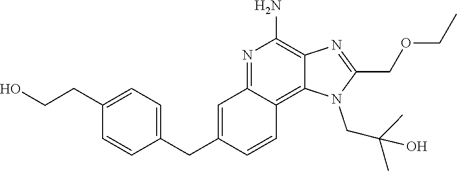 [SMILES: CCOCc1nc2c(N)nc3cc(Cc4ccc(CCO)cc4)ccc3c2n1CC(C)(C)O] |
| 126. | 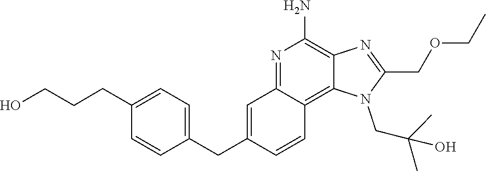 [SMILES: CCOCc1nc2c(N)nc3cc(Cc4ccc(CCCO)cc4)ccc3c2n1CC(C)(C)O] |
| 127. | 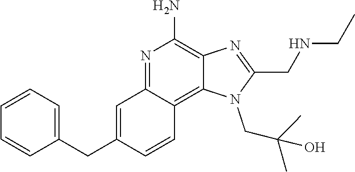 [SMILES: CCNCc1nc2c(N)nc3cc(Cc4ccccc4)ccc3c2n1CC(C)(C)O] |
| 128. | 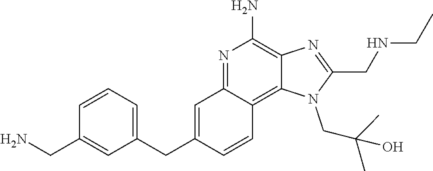 [SMILES: CCNCc1nc2c(N)nc3cc(Cc4cccc(CN)c4)ccc3c2n1CC(C)(C)O] |
| 129. | 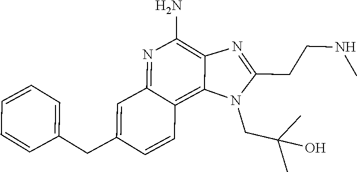 [SMILES: CNCCc1nc2c(N)nc3cc(Cc4ccccc4)ccc3c2n1CC(C)(C)O] |
| 130. | 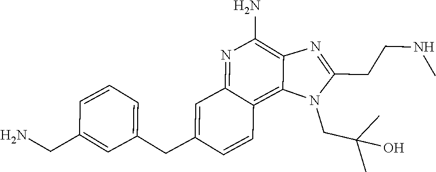 [SMILES: CNCCc1nc2c(N)nc3cc(Cc4cccc(CN)c4)ccc3c2n1CC(C)(C)O] |
| 131. | 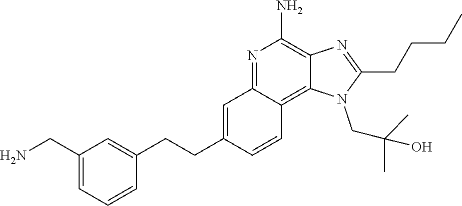 [SMILES: CCCCc1nc2c(N)nc3cc(CCc4cccc(CN)c4)ccc3c2n1CC(C)(C)O] |
| 132. | 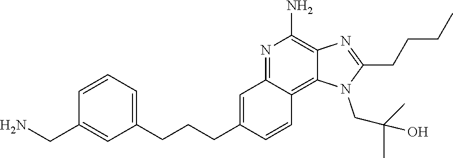 [SMILES: CCCCc1nc2c(N)nc3cc(CCCc4cccc(CN)c4)ccc3c2n1CC(C)(C)O] |
| 133. | 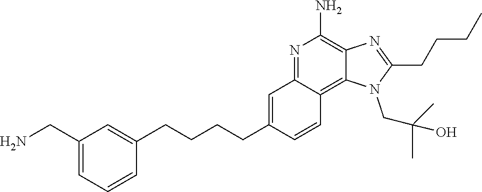 [SMILES: CCCCc1nc2c(N)nc3cc(CCCCc4cccc(CN)c4)ccc3c2n1CC(C)(C)O] |
| 134. | 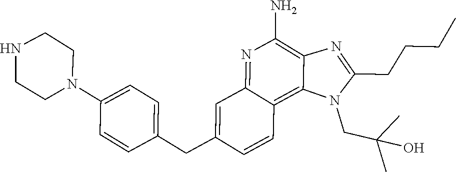 [SMILES: CCCCc1nc2c(N)nc3cc(Cc4ccc(N5CCNCC5)cc4)ccc3c2n1CC(C)(C)O] |
| 135. | 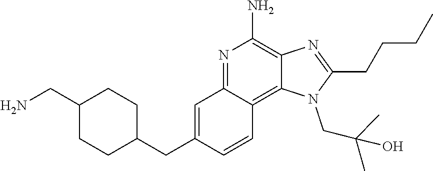 [SMILES: CCCCc1nc2c(N)nc3cc(CC4CCC(CN)CC4)ccc3c2n1CC(C)(C)O] |
| 136. | 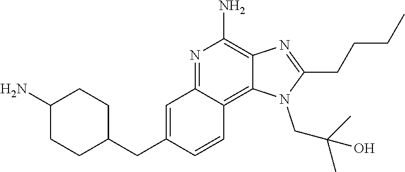 [SMILES: CCCCc1nc2c(N)nc3cc(CC4CCC(N)CC4)ccc3c2n1CC(C)(C)O] |
| 137. | 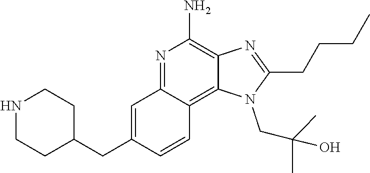 [SMILES: CCCCc1nc2c(N)nc3cc(CC4CCNCC4)ccc3c2n1CC(C)(C)O] |
| 138. | 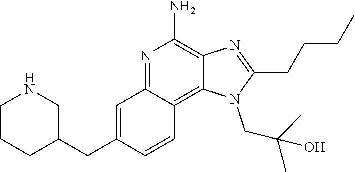 [SMILES: CCCCc1nc2c(N)nc3cc(CC4CCCNC4)ccc3c2n1CC(C)(C)O] |
| 139. | 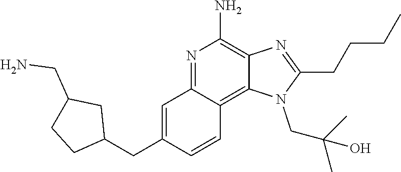 [SMILES: CCCCc1nc2c(N)nc3cc(CC4CCC(CN)C4)ccc3c2n1CC(C)(C)O] |
| 140. | 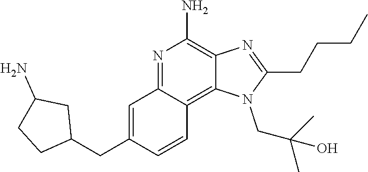 [SMILES: CCCCc1nc2c(N)nc3cc(CC4CCC(N)C4)ccc3c2n1CC(C)(C)O] |
| 141. | 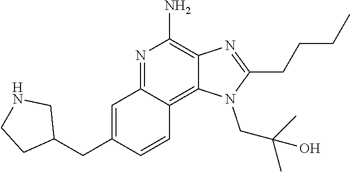 [SMILES: CCCCc1nc2c(N)nc3cc(CC4CCNC4)ccc3c2n1CC(C)(C)O] |
| 142. | 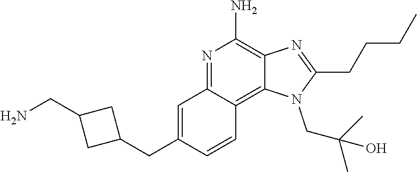 [SMILES: CCCCc1nc2c(N)nc3cc(CC4CC(CN)C4)ccc3c2n1CC(C)(C)O] |
| 143. | 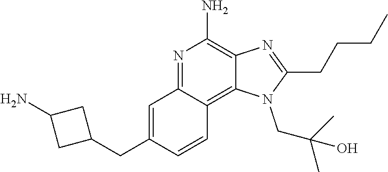 [SMILES: CCCCc1nc2c(N)nc3cc(CC4CC(N)C4)ccc3c2n1CC(C)(C)O] |
| 144. | 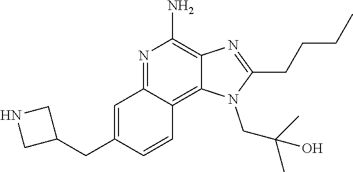 [SMILES: CCCCc1nc2c(N)nc3cc(CC4CNC4)ccc3c2n1CC(C)(C)O] |
| 145. | 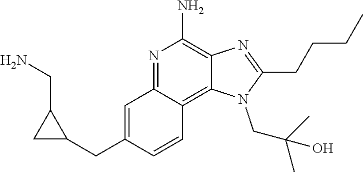 [SMILES: CCCCc1nc2c(N)nc3cc(CC4CC4CN)ccc3c2n1CC(C)(C)O] |
| 146. | 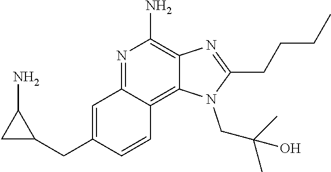 [SMILES: CCCCc1nc2c(N)nc3cc(CC4CC4N)ccc3c2n1CC(C)(C)O] |
| 147. | 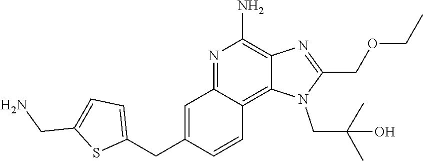 [SMILES: CCOCc1nc2c(N)nc3cc(Cc4ccc(CN)s4)ccc3c2n1CC(C)(C)O] |
| 148. | 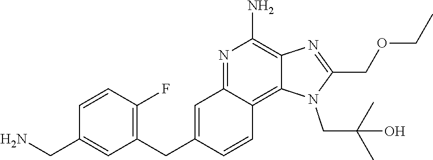 [SMILES: CCOCc1nc2c(N)nc3cc(Cc4cc(CN)ccc4F)ccc3c2n1CC(C)(C)O] |
| 149. | 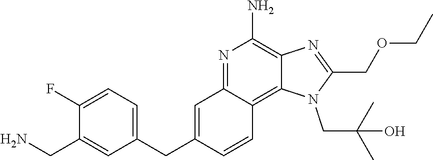 [SMILES: CCOCc1nc2c(N)nc3cc(Cc4ccc(F)c(CN)c4)ccc3c2n1CC(C)(C)O] |
| 150. | 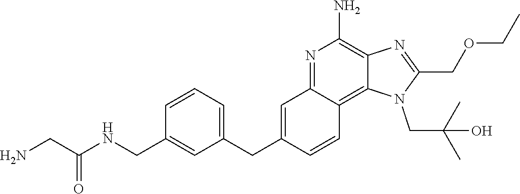 [SMILES: CCOCc1nc2c(N)nc3cc(Cc4cccc(CNC(=O)CN)c4)ccc3c2n1CC(C)(C)O] |
| 151. | 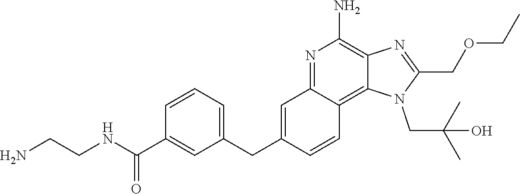 [SMILES: CCOCc1nc2c(N)nc3cc(Cc4cccc(C(=O)NCCN)c4)ccc3c2n1CC(C)(C)O] |
| 152. | 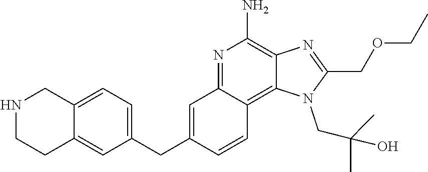 [SMILES: CCOCc1nc2c(N)nc3cc(Cc4ccc5c(c4)CCNC5)ccc3c2n1CC(C)(C)O] |
| 153. | 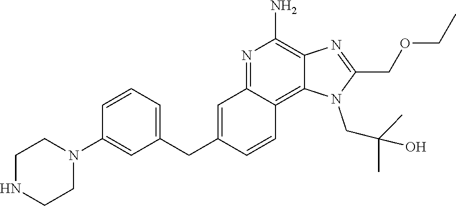 [SMILES: CCOCc1nc2c(N)nc3cc(Cc4cccc(N5CCNCC5)c4)ccc3c2n1CC(C)(C)O] |
| 154. | 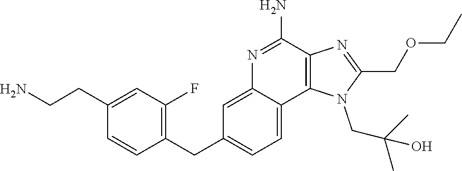 [SMILES: CCOCc1nc2c(N)nc3cc(Cc4ccc(CCN)cc4F)ccc3c2n1CC(C)(C)O] |
| 155. | 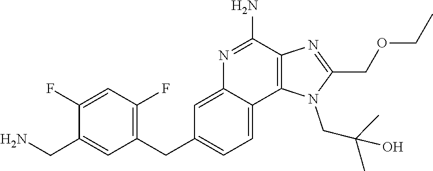 [SMILES: CCOCc1nc2c(N)nc3cc(Cc4cc(CN)c(F)cc4F)ccc3c2n1CC(C)(C)O] |
| 156. | 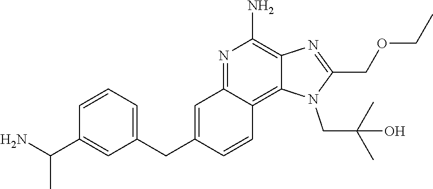 [SMILES: CCOCc1nc2c(N)nc3cc(Cc4cccc(C(C)N)c4)ccc3c2n1CC(C)(C)O] |
| 157. | 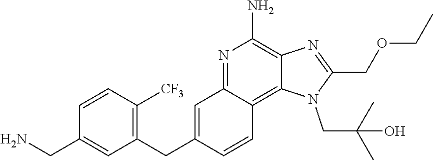 [SMILES: CCOCc1nc2c(N)nc3cc(Cc4cc(CN)ccc4C(F)(F)F)ccc3c2n1CC(C)(C)O] |
| 158. | 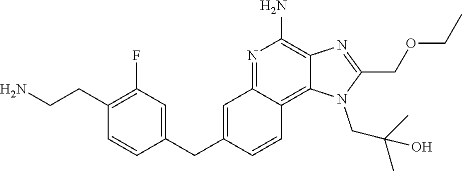 [SMILES: CCOCc1nc2c(N)nc3cc(Cc4ccc(CCN)c(F)c4)ccc3c2n1CC(C)(C)O] |
| 159. | 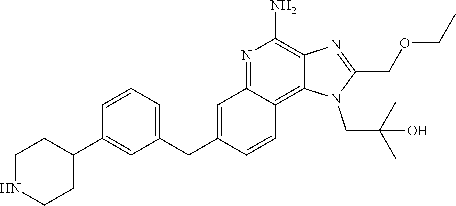 [SMILES: CCOCc1nc2c(N)nc3cc(Cc4cccc(C5CCNCC5)c4)ccc3c2n1CC(C)(C)O] |
| 160. | 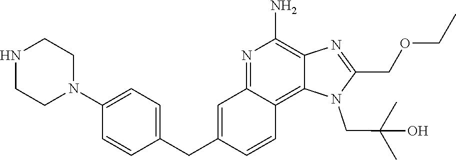 [SMILES: CCOCc1nc2c(N)nc3cc(Cc4ccc(N5CCNCC5)cc4)ccc3c2n1CC(C)(C)O] |
| 161. | 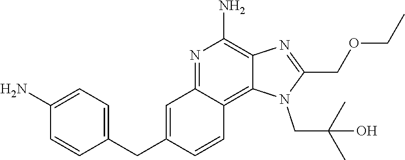 [SMILES: CCOCc1nc2c(N)nc3cc(Cc4ccc(N)cc4)ccc3c2n1CC(C)(C)O] |
| 162. | 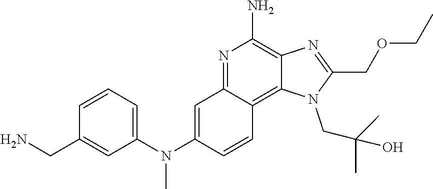 [SMILES: CCOCc1nc2c(N)nc3cc(N(C)c4cccc(CN)c4)ccc3c2n1CC(C)(C)O] |
| 163. | 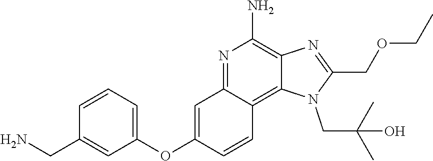 [SMILES: CCOCc1nc2c(N)nc3cc(Oc4cccc(CN)c4)ccc3c2n1CC(C)(C)O] |
| 164. | 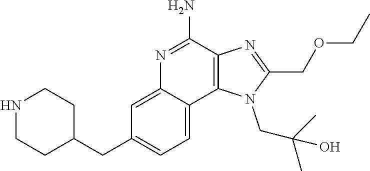 [SMILES: CCOCc1nc2c(N)nc3cc(CC4CCNCC4)ccc3c2n1CC(C)(C)O] |
| 165. | 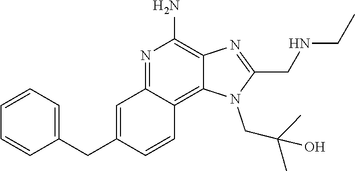 [SMILES: CCNCc1nc2c(N)nc3cc(Cc4ccccc4)ccc3c2n1CC(C)(C)O] |
| 166. | 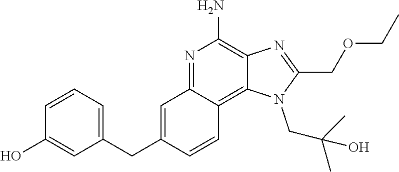 [SMILES: CCOCc1nc2c(N)nc3cc(Cc4cccc(O)c4)ccc3c2n1CC(C)(C)O] |
| 167. | 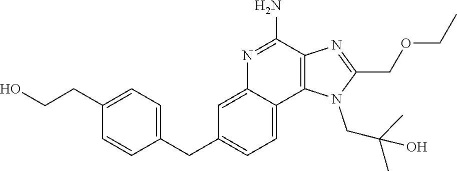 [SMILES: CCOCc1nc2c(N)nc3cc(Cc4ccc(CCO)cc4)ccc3c2n1CC(C)(C)O] |
| 168. | 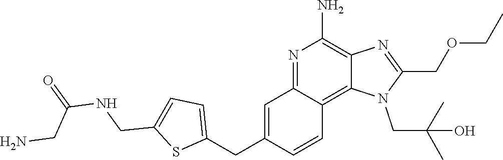 [SMILES: CCOCc1nc2c(N)nc3cc(Cc4ccc(CNC(=O)CN)s4)ccc3c2n1CC(C)(C)O] |
| 169. | 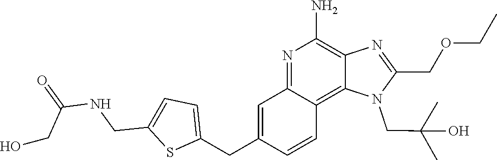 [SMILES: CCOCc1nc2c(N)nc3cc(Cc4ccc(CNC(=O)CO)s4)ccc3c2n1CC(C)(C)O] |
| 170. | 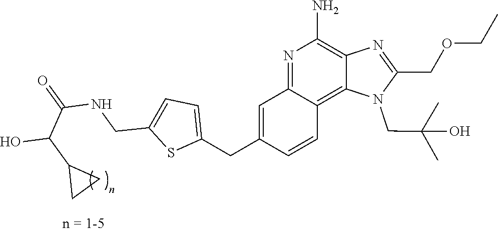 |
| 171. | 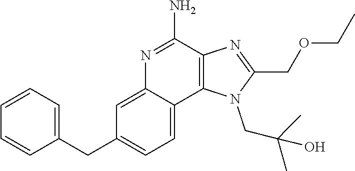 [SMILES: CCOCc1nc2c(N)nc3cc(Cc4ccccc4)ccc3c2n1CC(C)(C)O] |

The payload compound provided herein can further bonded to a linker provided herein to form a linker-payload compound.

The payload compounds provided herein are described with reference to both generic formulae and specific compounds. In addition, the payload compounds of the present disclosure may exist in a number of different forms or derivatives, all within the scope of the present disclosure. These include, for example, tautomers, stereoisomers, racemic mixtures, regioisomers, salts, solvated forms, amorphous forms, different crystal forms or polymorphs.

The payload provided herein or pharmaceutically acceptable salts thereof may contain one or more asymmetric centers and thus give rise to enantiomers, diastereomers, and other stereoisomeric forms that may be defined, in terms of absolute stereochemistry, as (R)- or (S)- or, as (D)- or (L)- for amino acids. The present disclosure includes all such possible isomers, as well as their racemic and optically pure forms. Optically active (+) and (−), (R)- and (S)-, or (D)- and (L)-isomers may be prepared using chiral synthons or chiral reagents, or resolved by conventional techniques, such as, chromatography and fractional crystallization. Traditional techniques for the preparation, isolation of individual enantiomers include chiral synthesis from a suitable optically pure precursor or resolution of the racemate (or the racemate of a salt or derivative) using, for example, chiral high pressure liquid chromatography (HPLC). When the compounds provided herein contain olefinic double bonds or other centers of geometric asymmetry, and unless specified otherwise, these compounds include both E and Z geometric isomers. As the same, all tautomeric forms are also intended to be included. Wherever compounds are represented in their chiral form, it is understood that the embodiment includes, but is not limited to, the specific diastereomerically or enantiomerically enriched form. In situations that the chirality is not specified but is present, it is understood that the embodiment is intended to include either the specific diastereomerically or enantiomerically enriched form; or a racemic or scalemic mixture of such compound(s). “Scalemic mixture” is a mixture of stereoisomers at a ratio other than 1:1.

The term “stereoisomer” refers to a compound containing the same atoms bonded by the same bonds but having different three-dimensional structures, which are not interchangeable. The current disclosure contemplates various stereoisomers and mixtures thereof and includes “enantiomers”, which refers to two stereoisomers whose molecules are non-superimposable mirror images of one another.

The term “enantiomers” represent a pair of stereoisomers that are non-superimposable mirror images of each other. A 1:1 mixture of a pair of enantiomers is a “racemic” mixture. A mixture of enantiomers at a ratio other than 1:1 is a “scalemic” mixture.

The term “diastereoisomers” represent stereoisomers that have at least two asymmetric atoms, but which are not mirror-images of each other.

“Tautomer” refers to a proton shift from one atom of a molecule to another atom of the same molecule. The present disclosure includes tautomers of any compounds provided herein.

Some payload compounds provided herein exist as tautomeric isomers. Tautomeric isomers are in equilibrium with one another. For example, amide containing compounds may exist in equilibrium with imidic acid tautomers. No matter which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. As the same, the imidic acid containing compounds are understood to include their amide tautomers.

Any formula or structure provided herein also represents unlabeled forms as well as isotopically labeled forms of the compounds. Isotopically labeled compounds have the same structures as depicted by the formulas given herein, except that one or more atoms are replaced by an atom having a selected atomic mass or mass number. Examples of isotopes include isotopes, such as, but not limited to, of hydrogen (²H (deuterium, D), ³H (tritium)), carbon (¹¹C, ¹³C, ¹⁴C), nitrogen (¹⁵N), oxygen (¹⁷O, ¹⁸O), phosphorous (³¹P, ³²P), fluorine (¹⁸F), chlorine (³⁶Cl), and iodine (¹²⁵I). Isotopically labelled compounds may have usages in metabolic studies, reaction kinetic studies, detection or imaging techniques, such as positron emission tomography (PET) or single-photon emission computed tomography (SPECT) in drug or substrate tissue distribution assays or in radioactive treatment of patients.

The payload compounds of the present disclosure can be formulated as or be in the form of pharmaceutically acceptable salts. Unless specified to the contrary, a compound provided herein includes pharmaceutically acceptable salts of such compound.

As used herein, the term “pharmaceutically acceptable” indicates that the substance or composition is compatible chemically and/or toxicologically, with the other ingredients comprising a formulation, and/or the subjects being treated therewith.

As used herein, the term “subject” refers to an animal, preferably a mammal, more preferably a human, who has been the object of treatment, observation or experiment.

As used herein, the term “pharmaceutically acceptable salt”, unless otherwise indicated, includes salts that retain the biological effectiveness of the free acids and bases of the specified compound and that are not biologically or otherwise undesirable. Contemplated pharmaceutically acceptable salt forms include, but are not limited to, mono, bis, tris, tetrakis, and so on. Pharmaceutically acceptable salts are non-toxic in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug.

Pharmaceutically acceptable salts include acid addition salts such as those containing sulfate, chloride, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethanolamine, t-butylamine, ethylenediamine, meglumine, procaine, aluminum, calcium, lithium, magnesium, potassium, sodium, ammonium, alkylamine, and zinc, when acidic functional groups, such as carboxylic acid or phenol are present. For example, see Remington's Pharmaceutical Sciences, 19^{th} ed., Mack Publishing Co., Easton, PA, Vol. 2, p. 1457, 1995; “Handbook of Pharmaceutical Salts: Properties, Selection, and Use” by Stahl and Wermuth, Wiley-VCH, Weinheim, Germany, 2002. Such salts can be prepared using the appropriate corresponding bases.

Pharmaceutically acceptable salts can be prepared by standard techniques. For example, the free-base form of a compound can be dissolved in a suitable solvent, such as an aqueous or aqueous-alcohol solution containing the appropriate acid and then isolated by evaporating the solution. Thus, if the particular compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

Similarly, if the particular compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as L-glycine, L-lysine, and L-arginine, ammonia, primary, secondary, and tertiary amines, and cyclic amines, such as hydroxyethylpyrrolidine, piperidine, morpholine or piperazine, and inorganic salts derived from sodium, calcium, potassium, magnesium, manganese, iron, copper, zinc, aluminum and lithium.

It is also to be understood that the payload compounds of present disclosure can exist in unsolvated forms, solvated forms (e.g., hydrated forms), and solid forms (e.g., amorphous, crystal or polymorphic forms), and the present disclosure is intended to encompass all such forms.

As used herein, the term “solvate” or “solvated form” refers to solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O. Examples of solvents that form solvates include, but are not limited to, water, isopropanol, ethanol, methanol, DMSO, ethyl acetate, acetic acid, and ethanolamine.

Since the payload compounds provided herein are intended for pharmaceutical use, they are preferably provided in substantially pure form, for example at least 60% pure, more suitably at least 75% pure, especially at least 98% pure (% are on a weight for weight basis).

The payload compounds provide herein show effective agonistic potency on TLR7 and/or TLR8. In some embodiments, the payload compounds provide herein show effective agonistic potency on both TLR7 and TLR8. In some embodiments, the payload compounds provide herein show selective agonistic potency on TLR7 over TLR8. In some embodiments, the payload compounds provide herein show selective agonistic potency on TLR8 over TLR7.

The payload compounds provided herein are particularly suitable for forming ADCs having TLR agonistic activity that are stable prior to administration to a subject. The targeting moiety and linker moiety useful for forming ADCs together with the payload compounds provided herein can be any targeting moieties and linker moieties known in the art.

### Synthesis of Payload Compounds

Synthesis of the payload compounds provided herein, including pharmaceutically acceptable salts thereof, are illustrated in the synthetic schemes in the examples. The payload compounds provided herein can be prepared using any known organic synthesis techniques and can be synthesized according to any of numerous possible synthetic routes, and thus these schemes are illustrative only and are not meant to limit other possible methods that can be used to prepare the compounds provided herein. Additionally, the steps in the Schemes are for better illustration and can be changed as appropriate. The embodiments of the payload compounds in examples were synthesized for the purposes of research and potentially submission to regulatory agencies.

The reactions for preparing the payload compounds of the present disclosure can be carried out in suitable solvents, which can be readily selected by one skilled in the art of organic synthesis. Suitable solvents can be substantially non-reactive with the starting materials (reactants), the intermediates, or products at the temperatures at which the reactions are carried out, e.g. temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected by one skilled in the art.

Preparation of the payload compounds of the present disclosure can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd Ed., Wiley & Sons, Inc., New York (1999), in P. Kocienski, Protecting Groups, Georg Thieme Verlag, 2003, and in Peter G. M. Wuts, Greene's Protective Groups in Organic Synthesis, 5^{th} Edition, Wiley, 2014, all of which are incorporated herein by reference in its entirety.

Reactions can be monitored according to any suitable method known in the art. For example, product formation can be monitored by spectroscopic means, such as nuclear magnetic resonance spectroscopy (e.g. ¹H or ¹³C), infrared spectroscopy, spectrophotometry (e.g. UV-visible), mass spectrometry, or by chromatographic methods such as high performance liquid chromatography (HPLC), liquid chromatography-mass spectroscopy (LCMS), or thin layer chromatography (TLC). Compounds can be purified by one skilled in the art by a variety of methods, including high performance liquid chromatography (HPLC) (“Preparative LC-MS Purification: Improved Compound Specific Method Optimization” Karl F. Blom, Brian Glass, Richard Sparks, Andrew P. Combs J. Combi. Chem. 2004, 6(6), 874-883, which is incorporated herein by reference in its entirety), and normal phase silica chromatography.

The known starting materials of the present disclosure can be synthesized by using or according to the known methods in the art, or can be purchased from commercial suppliers. Unless otherwise noted, analytical grade solvents and commercially available reagents were used without further purification.

Unless otherwise specified, the reactions of the present disclosure were all done under a positive pressure of nitrogen or argon or with a drying tube in anhydrous solvents, and the reaction flasks were typically fitted with rubber septa for the introduction of substrates and reagents via syringe. Glassware was oven dried and/or heat dried.

For illustrative purposes, the Examples section below shows synthetic route for preparing the payload compounds of the present disclosure as well as key intermediates. Those skilled in the art will appreciate that other synthetic routes may be used to synthesize the inventive compounds. Although specific starting materials and reagents are depicted, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

### Linker

In one aspect, there is provided a linker L having Formula (III):

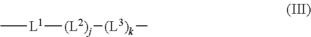

wherein
L¹ is a stretcher unit covalently attached to a targeting moiety;
L² is an optional peptide unit of two to twelve amino acid residues,
L³ is an optional spacer unit covalently attached to a payload unit, and
j and k are independently selected from 0 and 1.

In some embodiments, L¹ is selected from the group consisting of: has the formula:

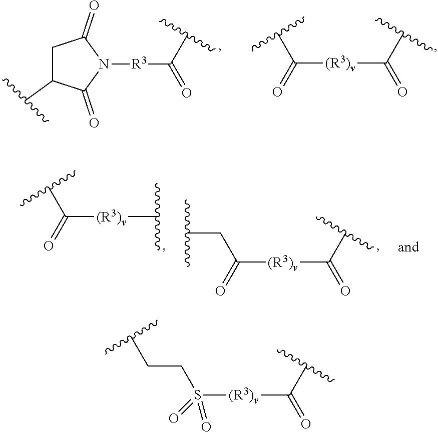

wherein each R³ is independently selected from the group consisting of alkyl, heteroalkyl, cycloalkyl, heterocyclyl, aryl, heteroaryl, alkylaryl, arylalkyl, alkylcycloalkyl, cycloalkylalkyl, alkylheterocyclyl, heterocyclylalkyl, -alkyl-C(O)N(R^{a})-alkyl-N(R^{a}), —N(R^{a})-alkyl-, and —(CH₂CH₂O)ᵣ—CH₂—, wherein R^{a} is H or alkyl, and r is an integer ranging from 1 to 10, v is an integer ranging from 0 to 5.

In certain embodiments, each R³ is independently selected from the group consisting of C₁₋₁₀ alkyl, C₁₋₈ heteroalkyl, C₃₋₈ cycloalkyl, 3 to 8 membered heterocyclyl, aryl, heteroaryl, (C₁₋₁₀ alkyl)aryl, aryl(C₁₋₁₀ alkyl), (C₁₋₁₀ alkyl)(C₃₋₈ cycloalkyl), (C₃₋₈ cycloalkyl)(C₁₋₁₀ alkyl), (C₁₋₁₀ alkyl)(3 to 8 membered heterocyclyl), (3 to 8 membered heterocyclyl)(C₁₋₁₀ alkyl), —(C₂₋₆ alkyl)-C(O)N(R^{a})—(C₂₋₆ alkyl)-N(R^{a}), —N(R^{a})—(C₂₋₆ alkyl)-, and —(CH₂CH₂O)ᵣ—CH₂—, wherein R^{a} is H or C₁₋₆ alkyl.

In certain embodiments, v is 1 and R³ is (CH₂)₅.

In some embodiments, j is 0 and k is 0.

In some embodiments, j is 0 and k is 1.

In some embodiments, L¹ has the formula:

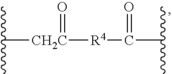

wherein R⁴ is selected from the group consisting of alkyl, -alkyl-O—, —N(R^{a})-alkyl-N(R^{a})—, —N(R^{a})-alkyl-, and (CH₂CH₂O)ᵣ—CH₂; wherein R^{a} is H or alkyl, and r is an integer ranging from 1 to 10.

In certain embodiments, L¹ has the formula:

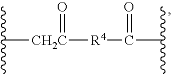

wherein R⁴ is selected from the group consisting of C₁₋₁₀ alkyl, —(C₁₋₁₀ alkyl)-O—, —N(R^{a})—(C₂₋₆ alkyl)-N(R^{a})—, —N(R^{a})—(C₂₋₆ alkyl)-, and —(CH₂CH₂O)ᵣ—CH₂—, wherein R^{a} is H or C₁₋₆ alkyl.

In some embodiments, L¹ has the formula:

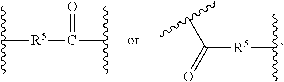

wherein R⁵ is selected from alkyl, -alkyl-O—, aryl, —N(R^{a})-alkyl- or —(CH₂CH₂O)ᵣ—CH₂—; wherein R^{a} is H or alkyl, and r is an integer ranging from 1 to 10.

In certain embodiments, L¹ has the formula:

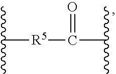

wherein R⁵ is selected from the group consisting of C₁₋₁₀ alkyl, —(C₁₋₁₀ alkyl)-O—, —N(R^{a})—(C₂₋₆ alkyl)— or —(CH₂CH₂O)_{Y}—CH₂—; wherein R^{a} is H or C₁₋₆ alkyl, and r is an integer ranging from 1 to 10.

In certain embodiments, L¹ has the formula:

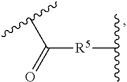

wherein R⁵ is selected from the group consisting of C₁₋₁₀ alkyl, —(C₁₋₁₀ alkyl)-O—, —N(R^{a})—(C₂₋₆ alkyl)— or —(CH₂CH₂O)ᵣ—CH₂—; wherein R^{a} is H or C₁₋₆ alkyl, and r is an integer ranging from 1 to 10.

In certain embodiments, the linker L forms a thioether bond with a cysteine amino acid of the targeting moiety, L¹ has the formula:

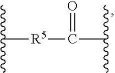

and R⁵ is —(C₂₋₆ alkyl)-O—, wherein the C₂₋₆ alkyl is optionally substituted with F, OH, O(C₁₋₆ alkyl), NH₂, NHCH₃, N(CH₃)₂, OP(O)₃H₂, and C₁₋₆ alkyl, wherein the C₁₋₆ alkyl is optionally substituted with one or more F.

In certain embodiments, the linker L forms an amide bond with a lysine amino acid of the targeting moiety, L¹ has the formula:

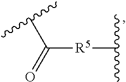

and R⁵ is —(C₂₋₆ alkyl)-O—, wherein the C₂₋₆ alkyl is optionally substituted with F, OH, O(C₁₋₆ alkyl), NH₂, NHCH₃, N(CH₃)₂, OP(O)₃H₂, and C₁₋₆ alkyl, wherein the C₁₋₆ alkyl is optionally substituted with one or more F.

In some embodiments, j is 1 and k is 1.

In some embodiments, j is 1 and L² comprises two or twelve amino acid residues independently selected from glycine, alanine, phenylalanine, lysine, arginine, valine, and citrulline.

In certain embodiments, L² is valine-citrulline.

In some embodiments, k is 1 and L³ comprises para-aminobenzyl or para-aminobenzyloxycarbonyl.

In certain embodiments, the linker L has the formula:

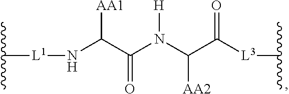

wherein AA1 and AA2 are independently selected from an amino acid side chain; p is an integer from 1 to 8.

In certain embodiments, the amino acid side chain is independently selected from H, —CH₃, —CH₂(C₆H₅), —CH₂CH₂CH₂CH₂NH₂, —CH₂CH₂CH₂NHC(NH)NH₂, —CHCH(CH₃)CH₃, and —CH₂CH₂CH₂NHC(O)NH₂.

In certain embodiments, the linker L has the formula:

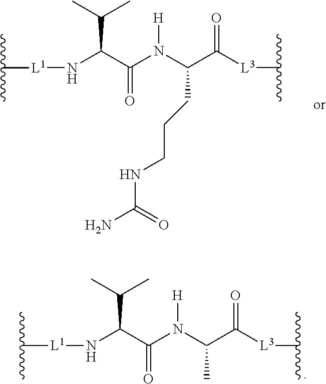

In certain embodiments, the linker L has the formula:

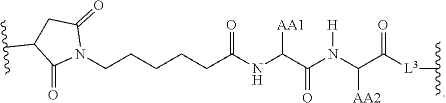

In certain embodiments, the linker L has the formula:

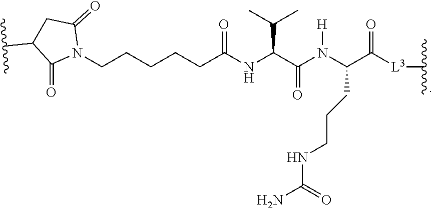

In certain embodiments, the linker L has the formula:

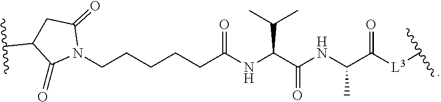

In certain embodiments, the linker L has the formula:

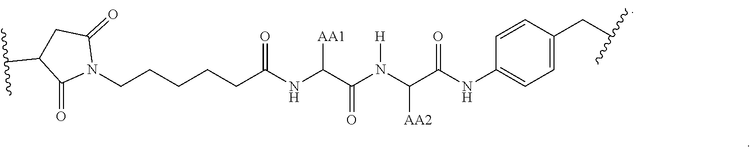

In certain embodiments, the linker L has the formula:

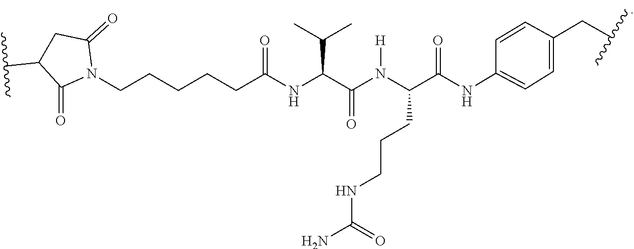 [SMILES: *Cc1ccc(NC(=O)[C@H](CCCNC(N)=O)NC(=O)[C@@H](NC(=O)CCCCCN2C(=O)CC(*)C2=O)C(C)C)cc1]

In certain embodiments, the linker L has the formula:

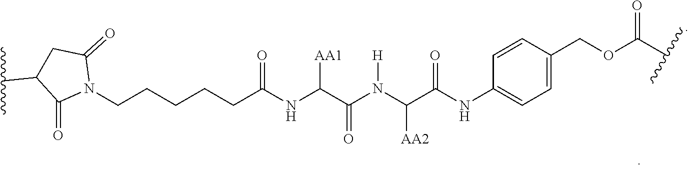

In certain embodiments, the linker L has the formula:

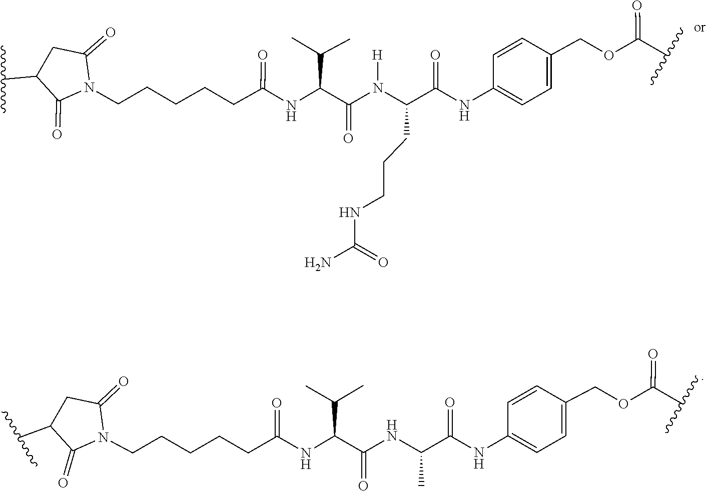 [SMILES: *C(=O)OCc1ccc(NC(=O)[C@H](CCCNC(N)=O)NC(=O)[C@@H](NC(=O)CCCCCN2C(=O)CC(*)C2=O)C(C)C)cc1 ; *C(=O)OCc1ccc(NC(=O)[C@H](C)NC(=O)[C@@H](NC(=O)CCCCCN2C(=O)CC(*)C2=O)C(C)C)cc1]

In certain embodiments, the linker L is selected from the group consisting of:

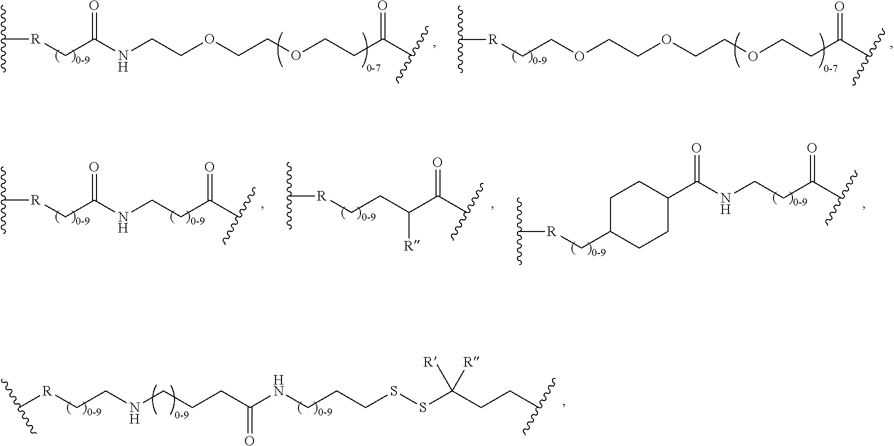

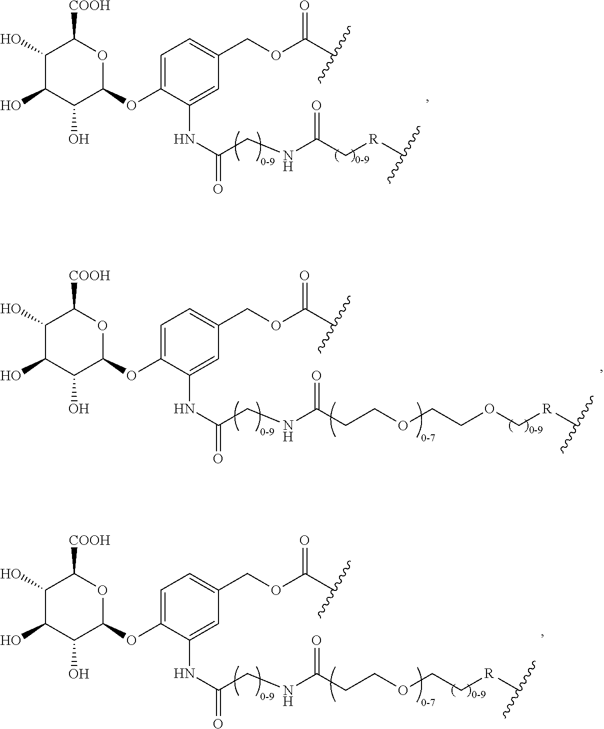

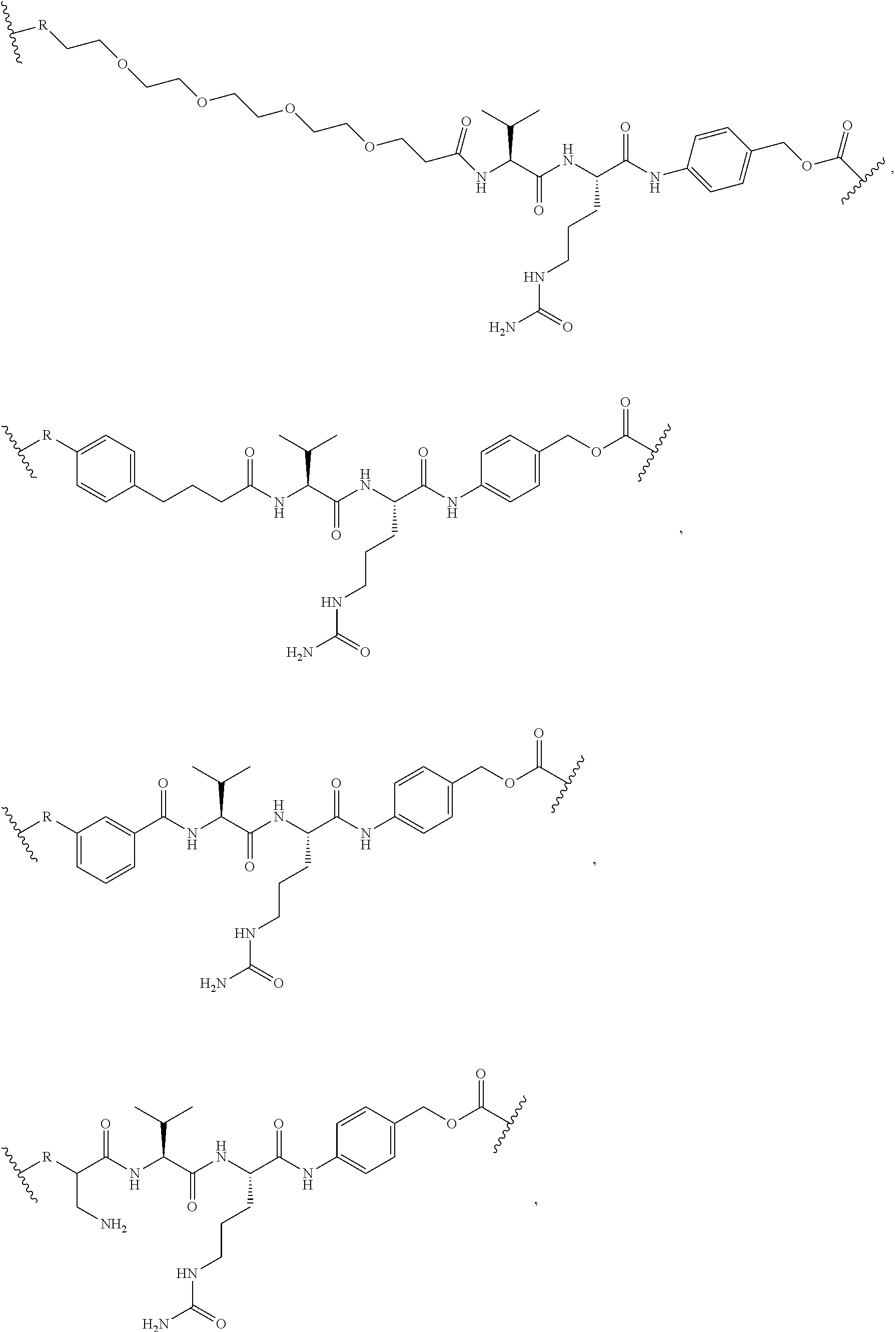

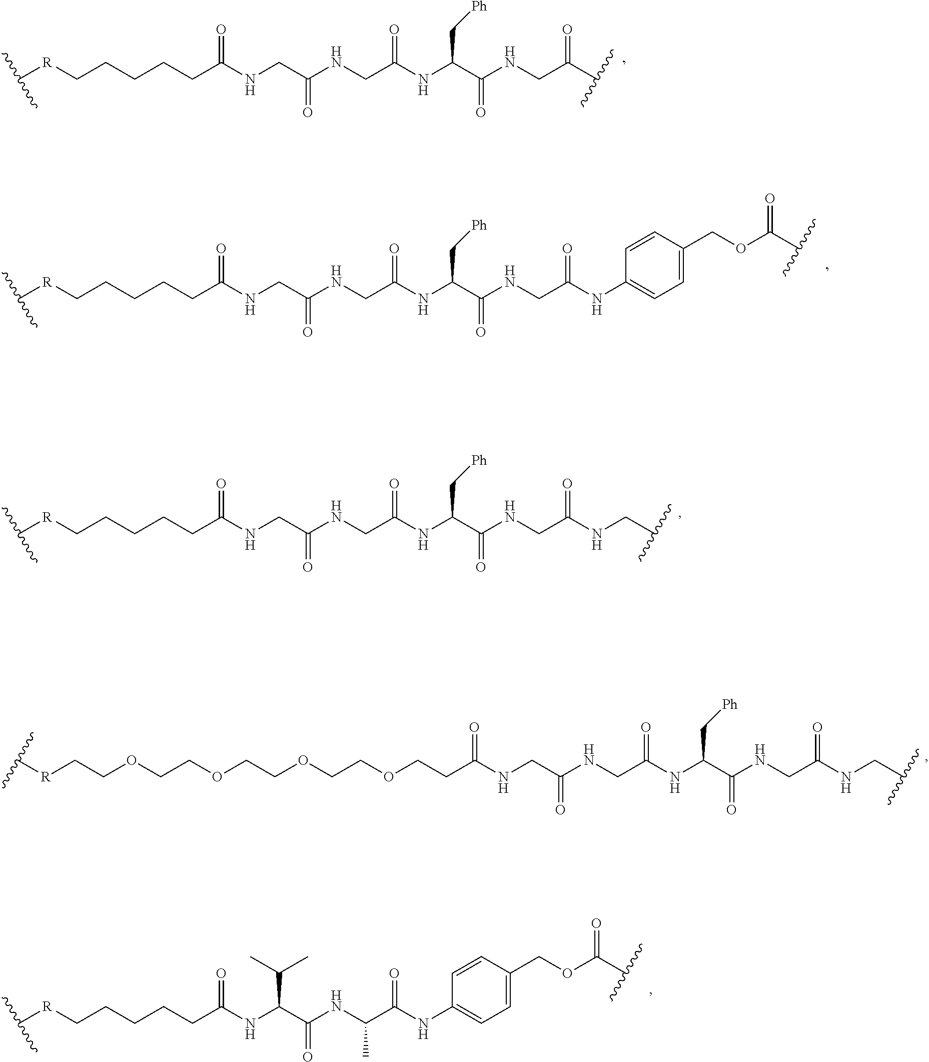

wherein R is selected from the group consisting of:

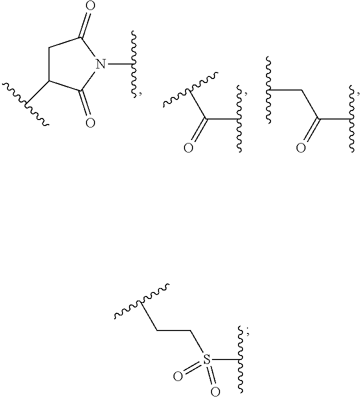

wherein each of R′ and R″ is independently hydrogen or methyl.

### Targeting Moiety

In one aspect, there is provided a targeting moiety comprises an immunoglobulin, a protein, a peptide, a small molecule, a nanoparticle, or a nucleic acid.

In some embodiments, the targeting moiety comprises an antibody or antigen binding fragment thereof.

In certain embodiments, the antibody specifically binds to one or more tumor-associated antigens or cell-surface receptors selected from BMPR1B, E16, STEAP1, MUC16, MPF, Napi2b, Sema 5b, PSCA hlg, ETBR, MSG783, STEAP2, TrpM4, CRIPTO, CD21, CD79b, FcRH2, HER2, NCA, MDP, IL20Ra, Brevican, EphB2R, ASLG659, PSCA, GEDA, BAFF-R, CD22, CD79a, CXCR5, HLA-DOB, P2X5, CD72, LY64, FcRHl, FcRH5, TENB2, PMEL17, TMEFF1, GDNF-Ral. Ly6E, TMEM46, Ly6G6D, LGR5, RET, LY6K, GPR19, GPR54, ASPHD1, Tyrosinase, TMEM118, GPR172A, CD33, and CLL-1.

In certain embodiments, the antibody specifically binds to HER2 or B7-H4.

In certain embodiments, the antibody specifically binds to HER2.

In some embodiments, the antibody is selected from the group consisting of Rituxan (rituximab), Herceptin (trastuzumab), Erbitux (cetuximab), Vectibix (Panitumumab), Arzerra (Ofatumumab), Benlysta (belimumab), Yervoy (ipilimumab), Perjeta (Pertuzumab), Tremelimumab, Nivolumab, Dacetuzumab, Urelumab, MPDL3280A, Lambrolizumab, and Blinatumomab.

In certain embodiments, the antibody is Herceptin (trastuzumab).

In certain embodiments, the antigen binding fragment is a Fab, Fab′, F(ab′)2, single domain antibody, T and Abs dimer, Fv, scFv, dsFv, ds-scFv, Fd, linear antibody, minibody, diabody, bispecific antibody fragment, bibody, tribody, sc-diabody, kappa (lamda) body, BiTE, DVD-Ig, SIP, SMIP, DART, polymer or aptamer.

In some embodiments, the targeting moiety comprises a protein, or a peptide.

In certain embodiments, the peptide comprises 2-100, 2-90, 2-80, 2-70, 2-60, 2-50, 2-40, 2-30, 2-25, 2-22, 2-20, 2-18, 2-15, 2-12, 2-10, 2-8, 4-50, 5-50, 5-40, 5-30, 5-25, 5-22, 5-20, 5-18, 5-15, 5-12, 5-10, 6, 7, 8, or 9 amino acids.

In some embodiments, the targeting moiety comprises a small molecule.

In certain embodiments, the small molecule targeting moiety is a folate or an analog thereof.

Folate is beneficial for forming a chemical bond with other groups due to its small molecule weight, non-immunogenicity, and good stability. Folate can be associated with a folate receptor expressed on a cell surface with a high affinity to mediate a cellular uptake of the folate. Although expressed at a very low level in most normal cells, a folate receptor is expressed at a high level in numerous cancer cells to meet the high folate demand of rapidly dividing cells under a low folate condition (see Kelemen L E, Int J Cancer, 2006; 119:243-50; Kane M A, et al., J Clin Invest. 1988; 81:1398-406; Matsue H, et al., Proc Natl Acad Sci USA. 1992; 89: 6006-9; Zhao R, et al., Annu Rev Nutr. 2011; 31:177-201). Folate is capable of specifically binding to a folate receptor on a cell surface, and is also capable of mediating endocytosis of a conjugate compound or a pharmaceutically acceptable salt thereof into target cells.

In certain embodiments, the analog of folate is selected from the group consisting of 5-methyltetrahydrofolate, 5-formyltetrahydrofolate, methotrexate, and 5,10-methylenetetrahydrofolate.

In some embodiments, the targeting moiety comprises a nanoparticle, preferably a targeted nanoparticle that attached to a targeting molecule that can binds specifically or preferably to a target. In some embodiments, the targeting nanoparticle by itself guides the compound of the present invention (such as by enrichment in tumor cells or tissue) and there are is no additional targeting molecules attached therein.

In some embodiments, the targeting moiety comprises a nucleic acid.

In some embodiments, the targeting moiety comprises a cell-interacting molecule.

In some embodiments, the targeting moiety comprises a ligand capable of binding to a cell surface receptor or other molecules.

In certain embodiments, the cell surface protein or marker of the present disclosure is a cell surface receptor.

In certain embodiments, the cell surface receptor of the present disclosure is selected from the group consisting of a Toll-like receptor (TLR), a transferrin receptor (TFR), a low-density lipoprotein receptor (LDLR), a folate receptor (FR), a growth hormone-inhibiting hormone receptor, a uric acid kinase receptor, a tumor necrosis factor receptor (TNFR), an integrin receptor (LFA-1), an SST-14 receptor (SSTR2), a GNRH receptor (GNRHR), a TRPV6 and an integrin α receptor.

In certain embodiments, the cell surface protein or marker of the present disclosure is a cell surface antigen.

In some embodiments, the targeting moiety is capable of binding to a tumor antigen.

In certain embodiments, the targeting moiety binds to a molecule selected from the group consisting of: CD2, CD19, CD20, CD22, CD27, CD28, CD33, CD37, CD38, CD40, CD40L, CD44, CD47, CD52, CD56, CD70, CD79, CD86/80, CD 113, CD122, CD137, CD155, CD160, CD206, 4-1BB, 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbB1, ErbB2, ErbB3, ErbB4, EGFL7, EpCAM, EphA2, EphA3, EphB2, FAP, Fibronectin, Folate Receptor, ASGPR, Ganglioside GM3, GD2, gp100, gpA33, GPNMB, ICOS, IGF1R, Integrin av, Integrin ανβ, KIR, LAG-3, Lewis Y, Mesothelin, c-MET, RON, PRLR, MN Carbonic anhydrase IX, MUC1, MUC16, Nectin-4, NKGD2, NOTCH, OX40, OX40L, PD-1, PDL1, PSCA, PSMA, RANKL, ROR1, ROR2, SLC44A4, Syndecan-1, TACI, TAG-72, Tenascin, TIM3, TREM-1, TREM-2, MACRO, Ly6E, TRAILR1, TRAILR2, VEGFR-1, VEGFR-2, VEGFR-3, FOLR1, TRPV6, FOLH1 (PMSA), GNRHR, Trop2, NECTIN4, LRP1, GLUT1, EGFR1, AXL, CA9, Claudin18.2, CLDN6, APN, DLL3, DLL4, CEACAM5, FZD10, TFRC, MET, SSTR2, CCKBR, LFA1, ICAM, GPR87, GM-CSF, GM-CSFR, CSF-1R, TLR family, GITRL, GITR, 4-BBL, ICOSL, MHCII antigen, TCR, FLT3, c-KIT, CTLA-4, IGIT, Galectin-9, HVEM, VISTA, B7-H4, phosphatidylserine, HHLA2, Galectin-3, LILRB2, LILRB3, LILRB4, SIGLEC15, CLEC5a, TIGIT, TfR, NKG2A, NKG2D, SLAMF7, KIR2DL1, KIR2DL2, KIR2DL3, FGFR1, FGFR2, FGFR4, NeuGcGM3, CXCR4 and variants thereof.

### Linker-Payload Compound

In another aspect, there is provided a linker-payload compound having Formula (Ia):

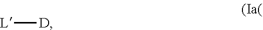

or a pharmaceutically acceptable salt thereof, wherein
L′ is a linker precursor;
D is a payload unit of Formula (II):

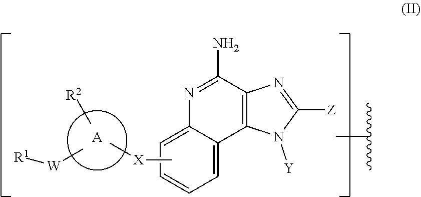

wherein:
   X is selected from the group consisting of —O—, —S—, —NH—, —(CH₂)ᵢ—, —(X¹)NC(O)—, —(X¹)NS(O)₂—, —C(O)N(X¹)— and —S(O)₂N(X¹)—, wherein —NH— and —(CH₂)ᵢ— are optionally substituted with one or more groups independently selected from halogen, hydroxyl, cyano, amino or alkyl;
   each X¹ is independently hydrogen, alkyl, alkenyl, or haloalkyl;
   Ring A is cycloalkyl, heterocyclyl, aryl or heteroaryl;
   W is selected from the group consisting of a direct bond, alkyl, cycloalkyl, heterocyclyl, *—W¹-alkyl-, *-alkyl-W¹—, and *-alkyl-W¹-alkyl-, wherein the alkyl, cycloalkyl, heterocyclyl, and alkyl in *—W¹-alkyl-, *-alkyl-W¹—, and *-alkyl-W¹-alkyl are optionally substituted with one or more groups independently selected from halogen, hydroxyl, cyano, amino, alkyl and —C(O)OR^{a}, and wherein * end of W is connected to Ring A;
   W¹ is —O—, —NR^{a}—, —C(O)—, —C(O)NR^{a}—, —OC(O)NR^{a}—, —NR^{a}C(O)—, —NR^{a}C(O)O—, or —NR^{a}C(O)NR^{a}—,
      each R^{a} is independently hydrogen, alkyl, or haloalkyl;
   R¹ is hydrogen, —N(R^{b})₂, hydroxyl or SH;
      each R^{b} is independently hydrogen, alkyl, or haloalkyl, or
      two R^{b} taken together with the nitrogen atom to which they are bound form a heterocyclyl;
   R² is hydrogen, halogen, hydroxyl, cyano, amino, alkyl, alkenyl, alkynyl, alkoxyl, haloalkyl, —OR^{e}, —OC(O)R^{e}, —SR^{e}, —B(OH)₂, —NO₂, —CHO, —C(═NOR^{e})(R^{e}), —R²⁻¹—N(R^{e})₂, —R²⁻¹—N(R^{e})C(O)R^{e}, —R²⁻¹—N(R^{e})S(O)₂R^{e}, —R²⁻¹—N(R^{e})P(O)₂R^{e}, —R²⁻¹—C(O)OR^{e}, —R²⁻¹—C(O)N(R^{e})₂, —R²⁻¹—S(O)₂N(R^{e})₂, —R²⁻¹—P(O)₂N(R^{e})₂, —OC(O)NR^{e}, or —NC(O)NR*,
      each R²⁻¹ is independently null or alkyl;
      each R^{e} is independently hydrogen, alkyl, or haloalkyl;
   Y is —Y¹—Y²—Y³, wherein
      Y¹ is a direct bond or —(CH₂)ₘ-Q¹-(CH₂)ₙ—O—*, wherein —(CH₂)ₘ— and —(CH₂)ₙ— are optionally substituted with one or more groups independently selected from halogen, hydroxyl, alkyl or haloalkyl, and * end of Y¹ is connected to Y²;
      Y² is a direct bond or —(CH₂)ₛ-Q²-(CH₂)ₜ—NR^{c}—**, wherein —(CH₂)ₛ— and —(CH₂)ₜ— are optionally substituted with one or more groups independently selected from halogen, hydroxyl, alkyl or haloalkyl, and ** end of Y² is connect to Y³;
      Y³ is selected from the group consisting of hydrogen, alkyl, -alkyl-aryl, -alkyl-N(R^{b})₂, —C(O)-alkyl, —C(O)-alkyl-N(R^{b})₂, and —S(O)₂-alkyl;
      Q¹ and Q² are each independently selected from a direct bond, cycloalkyl, heterocyclyl, aryl, or heteroaryl, wherein the cycloalkyl, heterocyclyl, aryl, and heteroaryl are optionally substituted with one or more groups independently selected from halogen, hydroxyl, cyano, amino, or alkyl;
      R^{c} is hydrogen or alkyl, or
      R^{c} and Y³ taken together with the atoms to which they are bound form a heterocyclyl optionally substituted with one or more groups independently selected from halogen, hydroxyl, cyano, amino or alkyl;
   Z is selected from the group consisting of alkyl, alkenyl, alkynyl, heteroalkyl, heteroalkenyl and heteroalkynyl, each of which is optionally substituted with one or more R^{d};
      R^{d} is selected from the group consisting of halogen, acyl, alkyl, alkenyl, alkynyl, cycloalkyl and —O-cycloalkyl;
   i is 0, 1, 2, 3, 4, 5 or 6;
   m is 0, 1, 2, 3, 4 or 5;
   n is 0, 1, 2, 3, 4 or 5;
   s is 0, 1, 2, 3, 4 or 5; and
   t is 0, 1,2, 3, 4 or 5.

In some embodiments, ring A is selected from the group consisting of:

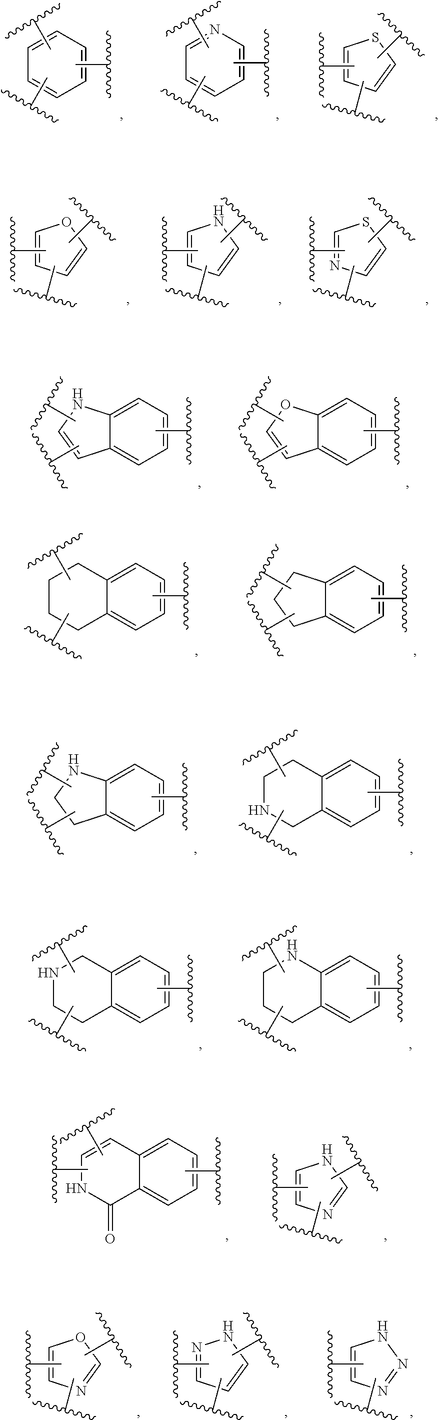

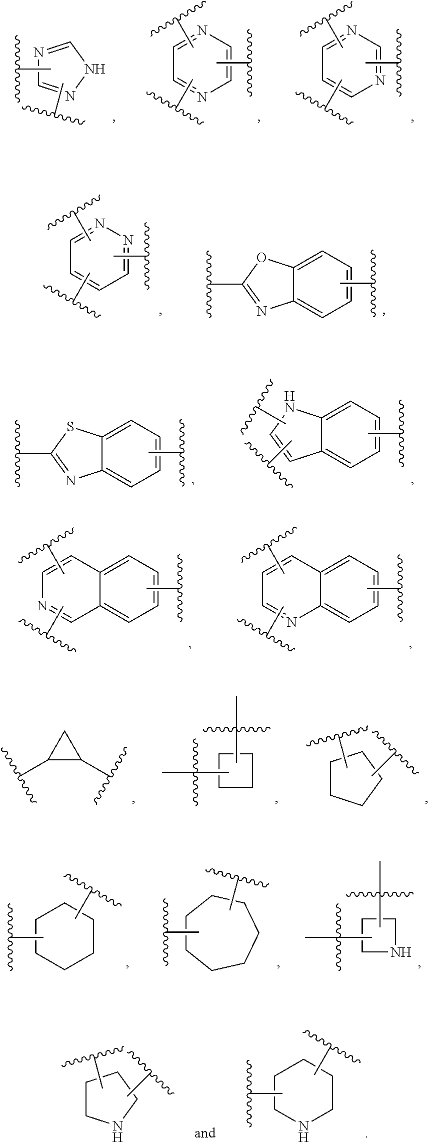

In some embodiments, X is —O—, —NH—, or —(CH₂)ᵢ—, wherein —NH— and —(CH₂)ᵢ— are optionally substituted with one or more halogen or alkyl.

In certain embodiments, X is —O—, —N(CH₃)—, —CH₂—, —(CH₂)₂—, —(CH₂)₃—, —(CH₂)₄—, —C(CH₃)₂— or —CF₂—.

In some embodiments, R¹ is hydrogen, —N(R^{b})₂, or hydroxyl.

In some embodiments, W is a direct bond.

In certain embodiments, W is a direct bond, R¹ is hydrogen, —N(R^{b})₂ or hydroxyl, and each R^{b} is independently hydrogen or alkyl, or two R^{b} taken together with the nitrogen atom to which they are bound form a heterocyclyl.

In some embodiments, W is alkyl optionally substituted with —C(O)OR^{a}. In certain embodiments, W is C₁₋₆ alkyl, C₁₋₅ alkyl, C₁₋₄ alkyl, C₁₋₃ alkyl, or C₁₋₂ alkyl, optionally substituted with —C(O)OR^{a}. In certain embodiments, W is methyl, ethyl or propyl, each optionally substituted with —C(O)OR^{a}. In certain embodiments, R^{a} is C₁₋₆ alkyl, C₁₋₅ alkyl, C₁₋₄ alkyl, C₁₋₃ alkyl, or C₁₋₂ alkyl.

In certain embodiments, W is alkyl optionally substituted with —C(O)OR^{a}, R¹ is hydrogen, hydroxyl or —N(R^{b})₂, and each R^{b} is independently hydrogen or alkyl, or two R^{b} taken together with the nitrogen atom to which they are bound form a heterocyclyl. In certain embodiments, R¹ is hydrogen. In certain embodiments, R¹ is hydroxyl. In certain embodiments, R¹ is —N(R^{b})₂, and each R^{b} is independently hydrogen, C₁₋₆ alkyl, C₁₋₅ alkyl, C₁₋₄ alkyl, C₁₋₃ alkyl, or C₁₋₂ alkyl. In certain embodiments, R¹ is —N(R^{b})₂, and each R^{b} is independently hydrogen or methyl. In certain embodiments, R¹ is —N(R^{b})₂, and two R^{b} taken together with the nitrogen atom to which they are bound form a 3 to 10 membered heterocyclyl, 3 to 9 membered heterocyclyl, 3 to 8 membered heterocyclyl, 3 to 7 membered heterocyclyl, 3 to 6 membered heterocyclyl, or 3 to 5 membered heterocyclyl.

In some embodiments, W is *—W¹-alkyl-. In certain embodiments, W is *—W¹—C₁₋₆ alkyl-, *—W¹—C₁₋₅ alkyl-, *—W¹—C₁₋₄ alkyl-, *—W¹—C₁₋₃ alkyl-, or *—W¹—C₁₋₂ alkyl-.

In certain embodiments, W is *—W¹-alkyl-, W¹ is —O—, —NR^{a}—, C(O)NR^{a}—OC(O)NR^{a}—, —NR^{a}C(O)—, —NR^{a}C(O)O—, or —NR^{a}C(O)NR^{a}—, and each R^{a} is independently hydrogen, alkyl, or haloalkyl.

In certain embodiments, W is *—W¹-alkyl-, and R¹ is —NH₂ or —NH—CH₃.

In some embodiments, W is *-alkyl-W¹—. In certain embodiments, W is *—C₁₋₆ alkyl-W¹—, *—C₁₋₅ alkyl-W¹—, *—C₁₋₄ alkyl-W¹—, *—C₁₋₃ alkyl-W¹—, or *—C₁₋₂ alkyl-W¹—.

In certain embodiments, W is *-alkyl-W¹—, and W¹ is —C(O)—.

In certain embodiments, W is *-alkyl-W¹—, W¹ is —C(O)—, and R¹ is —NH₂.

In some embodiments, W is *-alkyl-W¹-alkyl-. In certain embodiments, W is *-alkyl-W¹-alkyl-, and each alkyl in W is independently C₁₋₆ alkyl, C₁₋₅ alkyl, C₁₋₄ alkyl, C₁₋₃ alkyl, or C₁₋₂ alkyl.

In certain embodiments, W is *-alkyl-W¹-alkyl-, W¹ is —NR^{a}C(O)— or —OC(O)NR^{a}—, and R^{a} is hydrogen, alkyl, or haloalkyl.

In certain embodiments, W is *-alkyl-W¹-alkyl-, W¹ is —NR^{a}C(O)—, and R¹ is —NH₂.

In certain embodiments, W is *-alkyl-W¹-alkyl-, W¹ is —NR^{a}C(O)—, and R¹ is hydroxyl.

In embodiments, W is *-alkyl-W¹-alkyl-, W¹ is —OC(O)NR^{a}—, and R¹ is —NH—CH₃.

In some embodiments, W is cycloalkyl. In certain embodiments, W is C₃₋₁₀ cycloalkyl, C₃₋₉ cycloalkyl, C₃₋₈ cycloalkyl, C₃₋₇ cycloalkyl, C₃₋₆ cycloalkyl, or C₃₋₅ cycloalkyl.

In certain embodiments, W is cycloalkyl, and R¹ is —NH₂.

In some embodiment, W is heterocyclyl. In certain embodiments, W is 3 to 10 membered heterocyclyl, 3 to 9 membered heterocyclyl, 3 to 8 membered heterocyclyl, 3 to 7 membered heterocyclyl, 3 to 6 membered heterocyclyl, or 3 to 5 membered heterocyclyl.

In certain embodiments, W is heterocyclyl, and R¹ is hydrogen.

In some embodiments, R² is hydrogen, halogen, cyano, alkyl or alkoxyl. In certain embodiments, R² is hydrogen.

In some embodiments, Y¹ is a direct bond.

In certain embodiments, Y¹ is a direct bond, and Y² is a direct bond.

In certain embodiments, Y¹ is a direct bond, Y² is a direct bond, and Y³ is -alkyl-aryl.

In certain embodiments, Y¹ is a direct bond, and Y² is —(CH₂)ₛ-Q²-(CH₂)ₜ—NR^{c}—**. In certain embodiments, Q² is a direct bond, cycloalkyl or aryl, wherein the cycloalkyl or aryl is optionally substituted with one or more groups independently selected from halogen or alkyl. In certain embodiments, R¹ is hydrogen.

In certain embodiments, Y¹ is a direct bond, Y² is —(CH₂)ₛ-Q²-(CH₂)ₜ—NR^{c}—**, and Y³ is hydrogen, -alkyl-NH₂, or —S(O)₂-alkyl. In certain embodiments, the alkyl in -alkyl-NH₂ and —S(O)₂-alkyl is independently C₁₋₆ alkyl, C₁₋₅ alkyl, C₁₋₄ alkyl, C₁₃ alkyl, or C₁₋₂ alkyl.

In some embodiments, Y¹ is —(CH₂)ₘ-Q¹-(CH₂)ₙ—O—*, wherein —(CH₂)ₘ— and —(CH₂)ₙ— are optionally substituted with one or more groups independently selected from halogen, hydroxyl, alkyl or haloalkyl. In certain embodiments, —(CH₂)ₘ— and —(CH₂)ₙ— are optionally substituted with one or more groups independently selected from alkyl or haloalkyl. In certain embodiments, —(CH₂)ₘ— and —(CH₂)ₙ— are optionally substituted with one or more C₁₋₆ alkyl or C₁₋₆ haloalkyl. In certain embodiments, —(CH₂)ₘ— and —(CH₂)ₙ— are optionally substituted with one or more groups independently selected from methyl, ethyl or trifluoromethyl. In certain embodiments, Q¹ is a direct bond, cycloalkyl or aryl, wherein the cycloalkyl or aryl is optionally substituted with one or more groups independently selected from halogen or alkyl.

In certain embodiments, Y¹ is —(CH₂)ₘ-Q¹-(CH₂), —O—*, wherein —(CH₂)ₘ— and —(CH₂)ₙ— are optionally substituted with one or more groups independently selected from halogen, hydroxyl, alkyl or haloalkyl, and Y² is a direct bond. In certain embodiments, —(CH₂)ₘ— and —(CH₂)ₙ— are optionally substituted with one or more groups independently selected from alkyl or haloalkyl. In certain embodiments, —(CH₂)ₘ— and —(CH₂)ₙ— are optionally substituted with one or more C₁₋₆ alkyl or C₁₋₆ haloalkyl. In certain embodiments, —(CH₂)ₘ— and —(CH₂)ₙ— are optionally substituted with one or more groups independently selected from methyl, ethyl or trifluoromethyl.

In certain embodiments, Y¹ is —(CH₂)ₘ-Q¹-(CH₂)ₙ—O—*, Y² is a direct bond, and Y³ is hydrogen. In certain embodiments, —(CH₂)ₘ— and —(CH₂)ₙ— are optionally substituted with one or more groups independently selected from alkyl or haloalkyl. In certain embodiments, —(CH₂)ₘ— and —(CH₂)ₙ— are optionally substituted with one or more C₁₋₆ alkyl or C₁₋₆ haloalkyl. In certain embodiments, —(CH₂)ₘ— and —(CH₂)ₙ— are optionally substituted with one or more groups independently selected from methyl, ethyl or trifluoromethyl.

In certain embodiments, Y¹ is —(CH₂)ₘ-Q¹-(CH₂)ₙ—O—*, and Y² is —(CH₂)ₛ-Q²-(CH₂)ₜ—NR^{c}—**. In certain embodiments, Q² is a direct bond. In certain embodiments, R^{c} is hydrogen. In certain embodiments, —(CH₂)ₘ— and —(CH₂)ₙ— are optionally substituted with one or more groups independently selected from alkyl or haloalkyl. In certain embodiments, —(CH₂)ₘ— and —(CH₂)ₙ— are optionally substituted with one or more C₁₋₆ alkyl or C₁₋₆ haloalkyl. In certain embodiments, —(CH₂)ₘ— and —(CH₂)ₙ— are optionally substituted with one or more groups independently selected from methyl, ethyl or trifluoromethyl.

In certain embodiments, Y¹ is —(CH₂)ₘ-Q¹-(CH₂)ₙ—O—*, Y² is —(CH₂)ₛ-Q²-(CH₂)ₜ—NR^{c}—**, and Y³ is hydrogen, —C(O)-alkyl or —C(O)-alkyl-N(R^{b})₂. In certain embodiments, —(CH₂)ₘ— and —(CH₂)ₙ— are optionally substituted with one or more groups independently selected from alkyl or haloalkyl. In certain embodiments, —(CH₂)ₘ— and —(CH₂)ₙ— are optionally substituted with one or more C₁₋₆ alkyl or C₁₋₆ haloalkyl. In certain embodiments, —(CH₂)ₘ— and —(CH₂)ₙ— are optionally substituted with one or more groups independently selected from methyl, ethyl or trifluoromethyl. In certain embodiments, R^{c} and Y³ taken together with the atoms to which they are bound form a heterocyclyl optionally substituted with one or more halogen.

In some embodiments, Z is selected from the group consisting of alkyl, alkenyl, alkynyl and heteroalkyl, which is optionally substituted with one or more R^{d}. In certain embodiments, Z is selected from the group consisting of C₁₋₆ alkyl, C₁₋₆ alkenyl, C₁₋₆ alkynyl and C₁₋₆ heteroalkyl, which is optionally substituted with one or more R^{d}.

In certain embodiments, R^{d} is selected from the group consisting of halogen, acyl, alkyl, cycloalkyl and —O-cycloalkyl. In certain embodiments, R^{d} is selected from the group consisting of halogen, acyl, C₁₋₆ alkyl, C₃₋₆ cycloalkyl and —O—C₃₋₆ cycloalkyl.

In some embodiments, D is a payload unit of Formula (Iia):

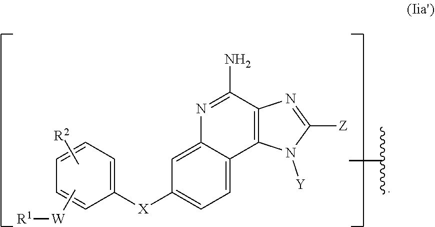

In some embodiments, the payload unit is selected from the group consisting of:

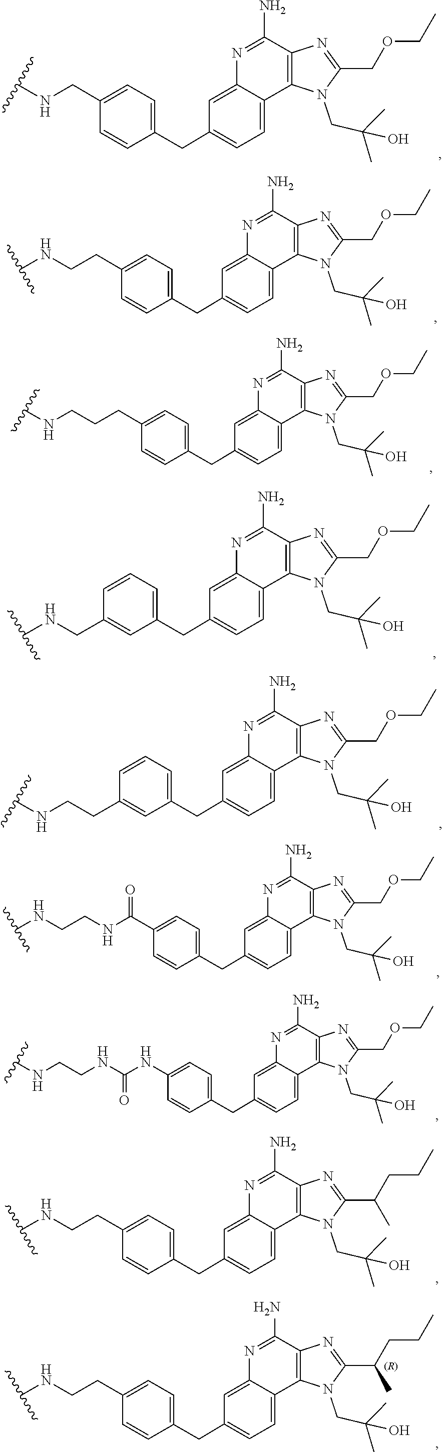

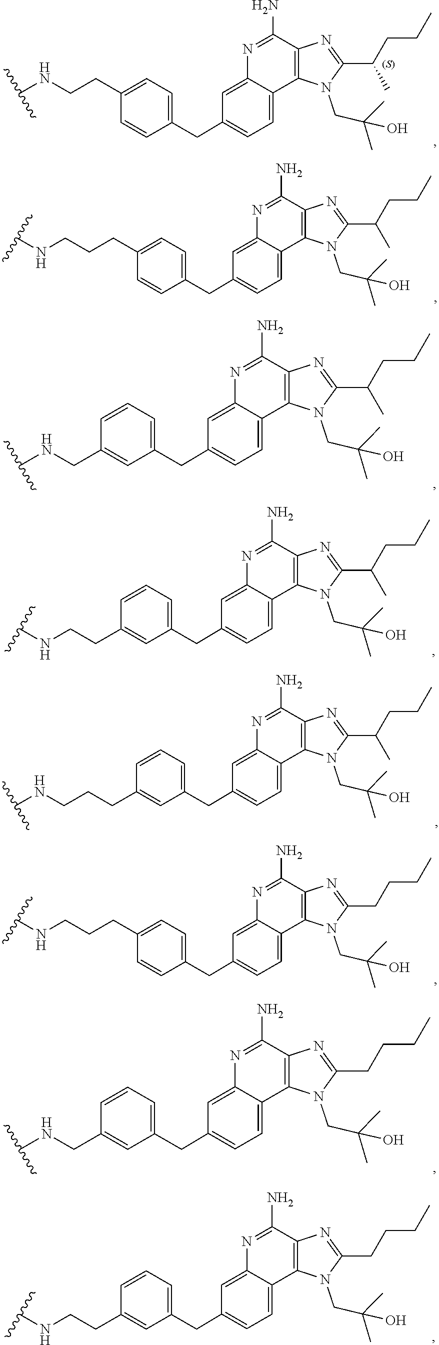

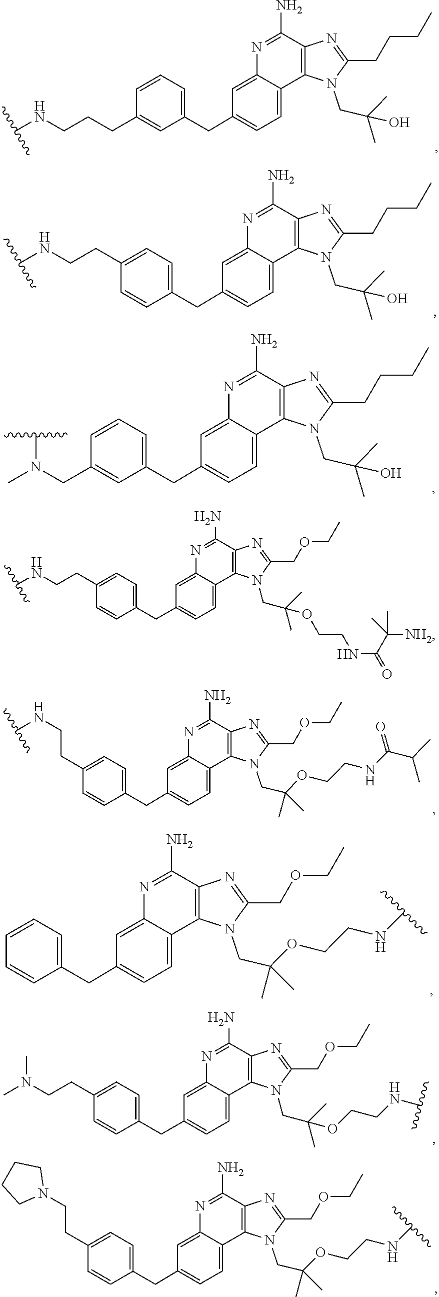

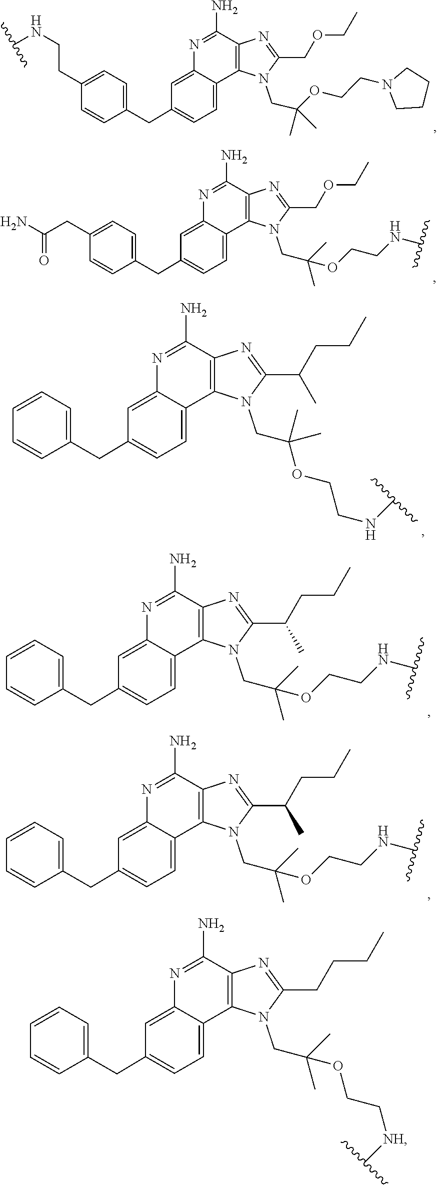

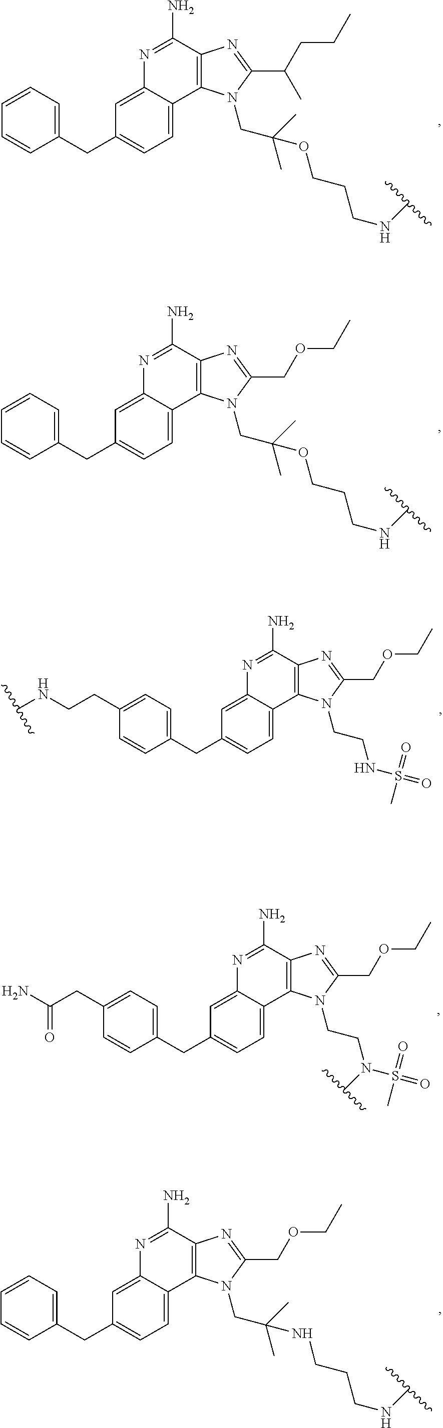 [SMILES: *NCCCOC(C)(C)Cn1c(C(C)CCC)nc2c(N)nc3cc(Cc4ccccc4)ccc3c21 ; *NCCCOC(C)(C)Cn1c(COCC)nc2c(N)nc3cc(Cc4ccccc4)ccc3c21 ; *NCCc1ccc(Cc2ccc3c(c2)nc(N)c2nc(COCC)n(CCNS(C)(=O)=O)c23)cc1 ; *N(CCn1c(COCC)nc2c(N)nc3cc(Cc4ccc(CC(N)=O)cc4)ccc3c21)S(C)(=O)=O ; *NCCCNC(C)(C)Cn1c(COCC)nc2c(N)nc3cc(Cc4ccccc4)ccc3c21]

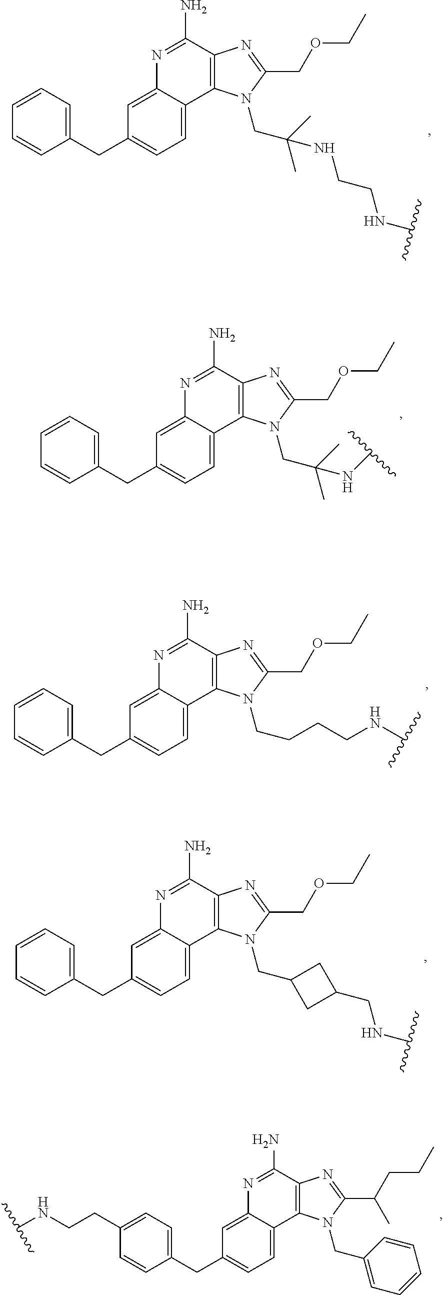 [SMILES: *NCCNC(C)(C)Cn1c(COCC)nc2c(N)nc3cc(Cc4ccccc4)ccc3c21 ; *NC(C)(C)Cn1c(COCC)nc2c(N)nc3cc(Cc4ccccc4)ccc3c21 ; *NCCCCn1c(COCC)nc2c(N)nc3cc(Cc4ccccc4)ccc3c21 ; *NCC1CC(Cn2c(COCC)nc3c(N)nc4cc(Cc5ccccc5)ccc4c32)C1 ; *NCCc1ccc(Cc2ccc3c(c2)nc(N)c2nc(C(C)CCC)n(Cc4ccccc4)c23)cc1]

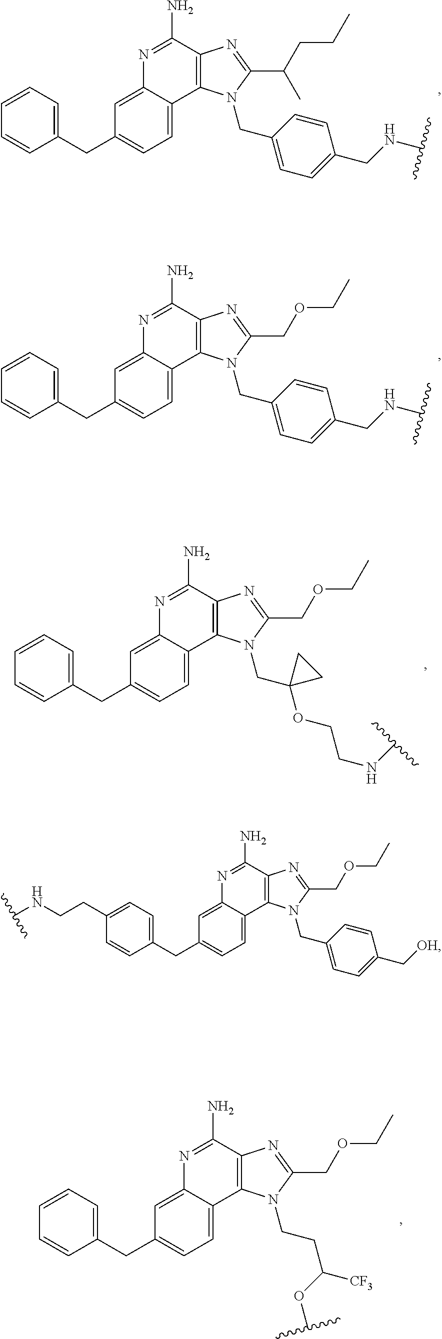 [SMILES: *NCc1ccc(Cn2c(C(C)CCC)nc3c(N)nc4cc(Cc5ccccc5)ccc4c32)cc1 ; *NCc1ccc(Cn2c(COCC)nc3c(N)nc4cc(Cc5ccccc5)ccc4c32)cc1 ; *NCCOC1(Cn2c(COCC)nc3c(N)nc4cc(Cc5ccccc5)ccc4c32)CC1 ; *NCCc1ccc(Cc2ccc3c(c2)nc(N)c2nc(COCC)n(Cc4ccc(CO)cc4)c23)cc1 ; *OC(CCn1c(COCC)nc2c(N)nc3cc(Cc4ccccc4)ccc3c21)C(F)(F)F]

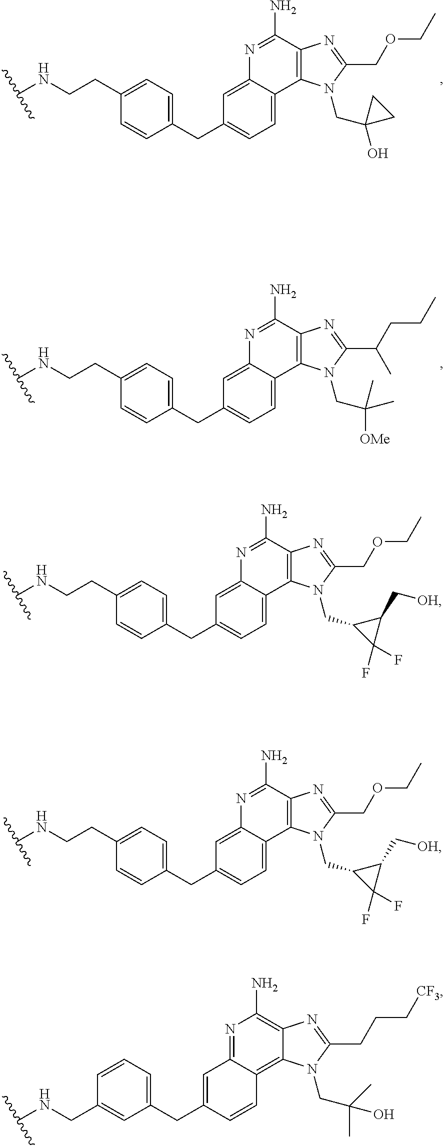 [SMILES: *NCCc1ccc(Cc2ccc3c(c2)nc(N)c2nc(COCC)n(CC4(O)CC4)c23)cc1 ; *NCCc1ccc(Cc2ccc3c(c2)nc(N)c2nc(C(C)CCC)n(CC(C)(C)OC)c23)cc1 ; *NCCc1ccc(Cc2ccc3c(c2)nc(N)c2nc(COCC)n(C[C@@H]4[C@@H](CO)C4(F)F)c23)cc1 ; *NCCc1ccc(Cc2ccc3c(c2)nc(N)c2nc(COCC)n(C[C@@H]4[C@H](CO)C4(F)F)c23)cc1 ; *NCc1cccc(Cc2ccc3c(c2)nc(N)c2nc(CCCC(F)(F)F)n(CC(C)(C)O)c23)c1]

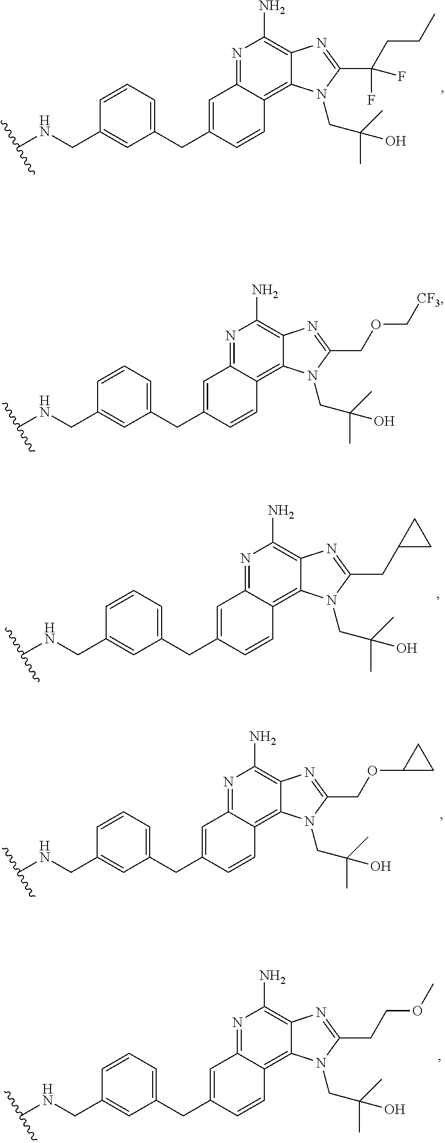 [SMILES: *NCc1cccc(Cc2ccc3c(c2)nc(N)c2nc(C(F)(F)CCC)n(CC(C)(C)O)c23)c1 ; *NCc1cccc(Cc2ccc3c(c2)nc(N)c2nc(COCC(F)(F)F)n(CC(C)(C)O)c23)c1 ; *NCc1cccc(Cc2ccc3c(c2)nc(N)c2nc(CC4CC4)n(CC(C)(C)O)c23)c1 ; *NCc1cccc(Cc2ccc3c(c2)nc(N)c2nc(COC4CC4)n(CC(C)(C)O)c23)c1 ; *NCc1cccc(Cc2ccc3c(c2)nc(N)c2nc(CCOC)n(CC(C)(C)O)c23)c1]

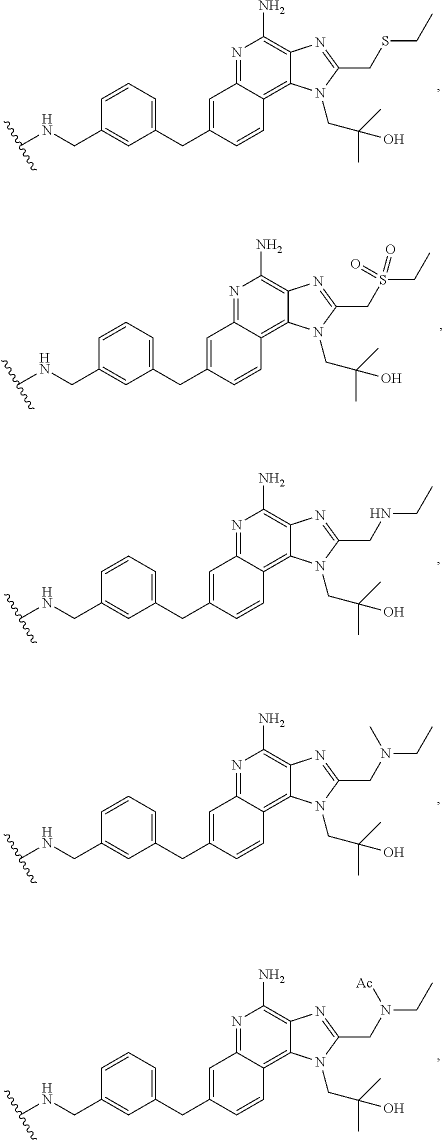 [SMILES: *NCc1cccc(Cc2ccc3c(c2)nc(N)c2nc(CSCC)n(CC(C)(C)O)c23)c1 ; *NCc1cccc(Cc2ccc3c(c2)nc(N)c2nc(CS(=O)(=O)CC)n(CC(C)(C)O)c23)c1 ; *NCc1cccc(Cc2ccc3c(c2)nc(N)c2nc(CNCC)n(CC(C)(C)O)c23)c1 ; *NCc1cccc(Cc2ccc3c(c2)nc(N)c2nc(CN(C)CC)n(CC(C)(C)O)c23)c1 ; *NCc1cccc(Cc2ccc3c(c2)nc(N)c2nc(CN(CC)C(C)=O)n(CC(C)(C)O)c23)c1]

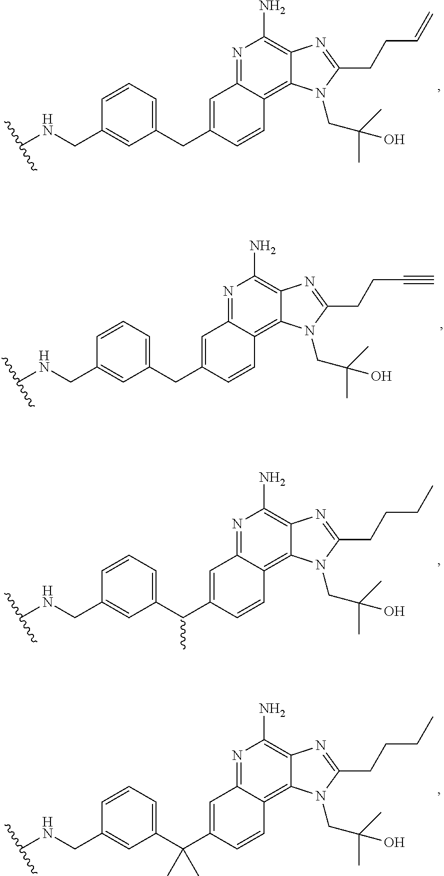 [SMILES: *NCc1cccc(Cc2ccc3c(c2)nc(N)c2nc(CCC=C)n(CC(C)(C)O)c23)c1 ; *NCc1cccc(Cc2ccc3c(c2)nc(N)c2nc(CCC#C)n(CC(C)(C)O)c23)c1 ; *NCc1cccc(C(C)c2ccc3c(c2)nc(N)c2nc(CCCC)n(CC(C)(C)O)c23)c1 ; *NCc1cccc(C(C)(C)c2ccc3c(c2)nc(N)c2nc(CCCC)n(CC(C)(C)O)c23)c1]

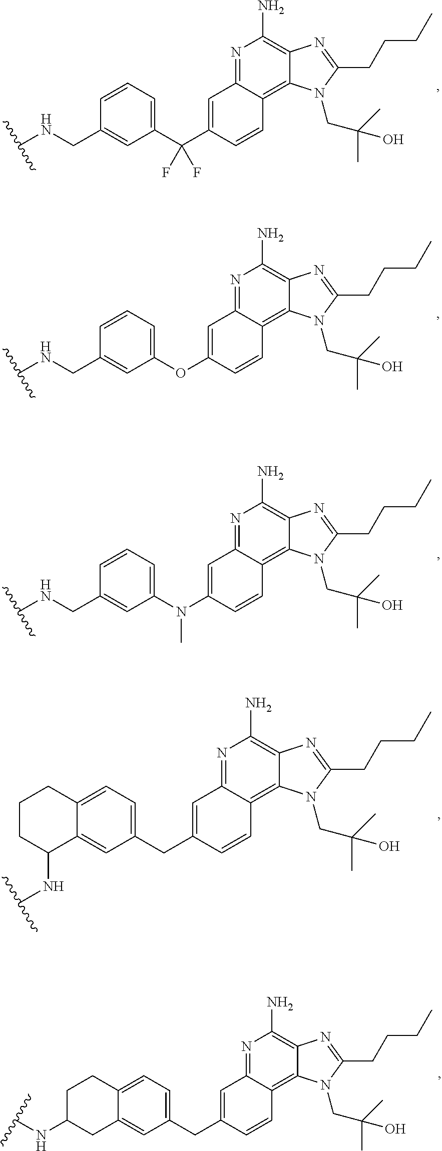 [SMILES: *NCc1cccc(C(F)(F)c2ccc3c(c2)nc(N)c2nc(CCCC)n(CC(C)(C)O)c23)c1 ; *NCc1cccc(Oc2ccc3c(c2)nc(N)c2nc(CCCC)n(CC(C)(C)O)c23)c1 ; *NCc1cccc(N(C)c2ccc3c(c2)nc(N)c2nc(CCCC)n(CC(C)(C)O)c23)c1 ; *NC1CCCc2ccc(Cc3ccc4c(c3)nc(N)c3nc(CCCC)n(CC(C)(C)O)c34)cc21 ; *NC1CCc2ccc(Cc3ccc4c(c3)nc(N)c3nc(CCCC)n(CC(C)(C)O)c34)cc2C1]

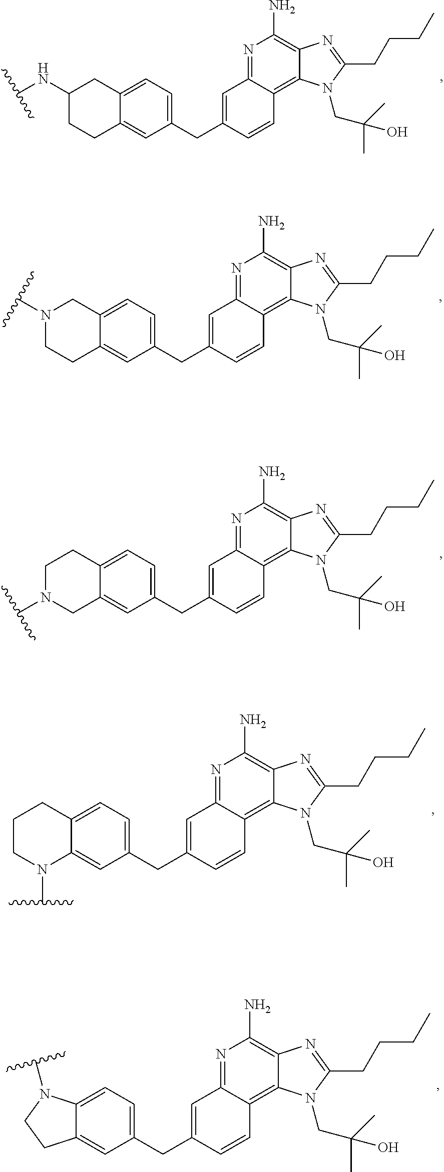 [SMILES: *NC1CCc2cc(Cc3ccc4c(c3)nc(N)c3nc(CCCC)n(CC(C)(C)O)c34)ccc2C1 ; *N1CCc2cc(Cc3ccc4c(c3)nc(N)c3nc(CCCC)n(CC(C)(C)O)c34)ccc2C1 ; *N1CCc2ccc(Cc3ccc4c(c3)nc(N)c3nc(CCCC)n(CC(C)(C)O)c34)cc2C1 ; *N1CCCc2ccc(Cc3ccc4c(c3)nc(N)c3nc(CCCC)n(CC(C)(C)O)c34)cc21 ; *N1CCc2cc(Cc3ccc4c(c3)nc(N)c3nc(CCCC)n(CC(C)(C)O)c34)ccc21]

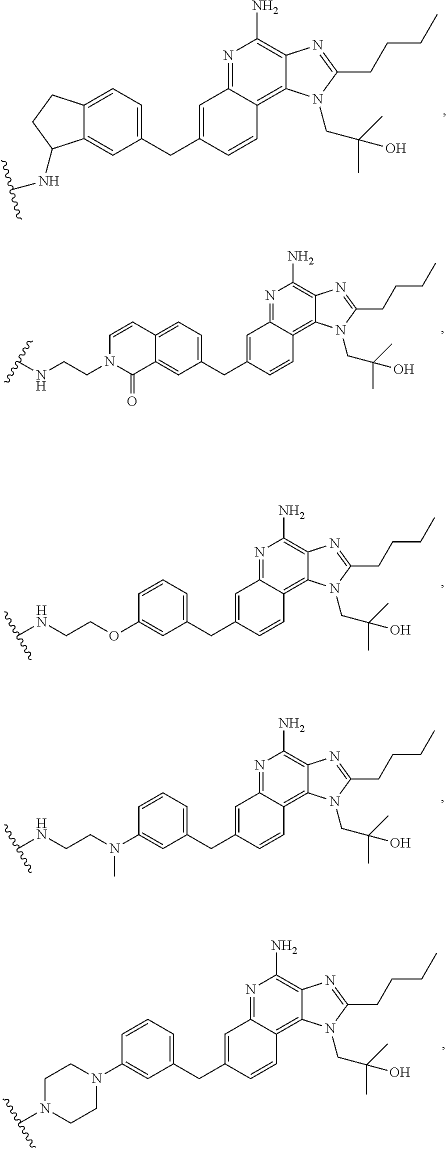 [SMILES: *NC1CCc2ccc(Cc3ccc4c(c3)nc(N)c3nc(CCCC)n(CC(C)(C)O)c34)cc21 ; *NCCn1ccc2ccc(Cc3ccc4c(c3)nc(N)c3nc(CCCC)n(CC(C)(C)O)c34)cc2c1=O ; *NCCOc1cccc(Cc2ccc3c(c2)nc(N)c2nc(CCCC)n(CC(C)(C)O)c23)c1 ; *NCCN(C)c1cccc(Cc2ccc3c(c2)nc(N)c2nc(CCCC)n(CC(C)(C)O)c23)c1 ; *N1CCN(c2cccc(Cc3ccc4c(c3)nc(N)c3nc(CCCC)n(CC(C)(C)O)c34)c2)CC1]

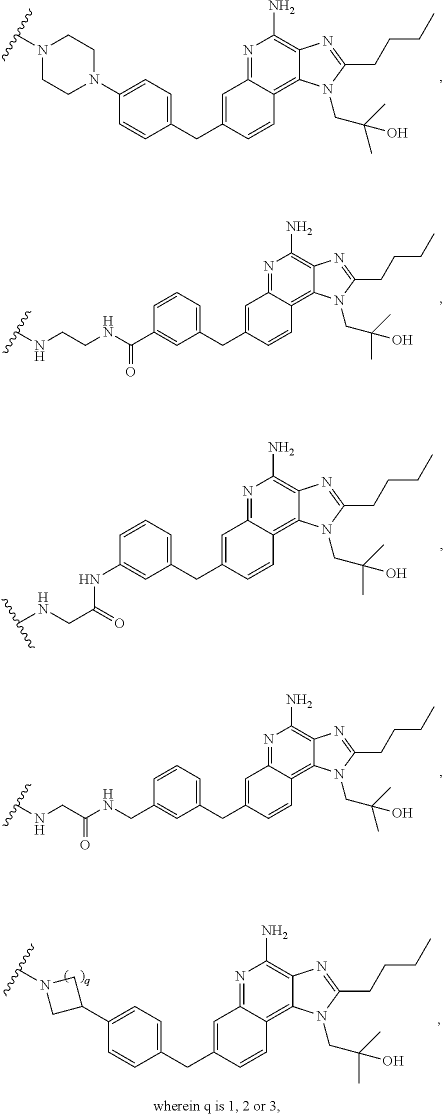

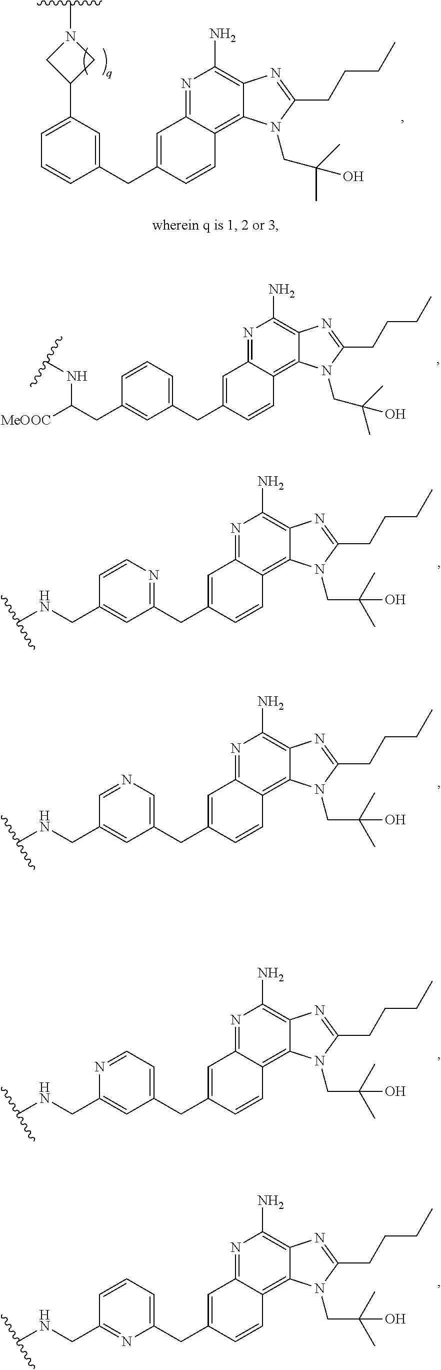

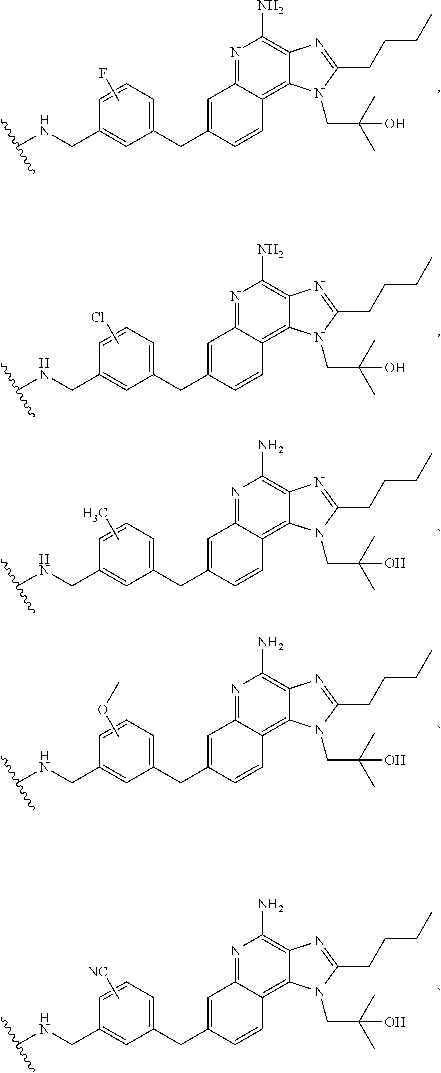

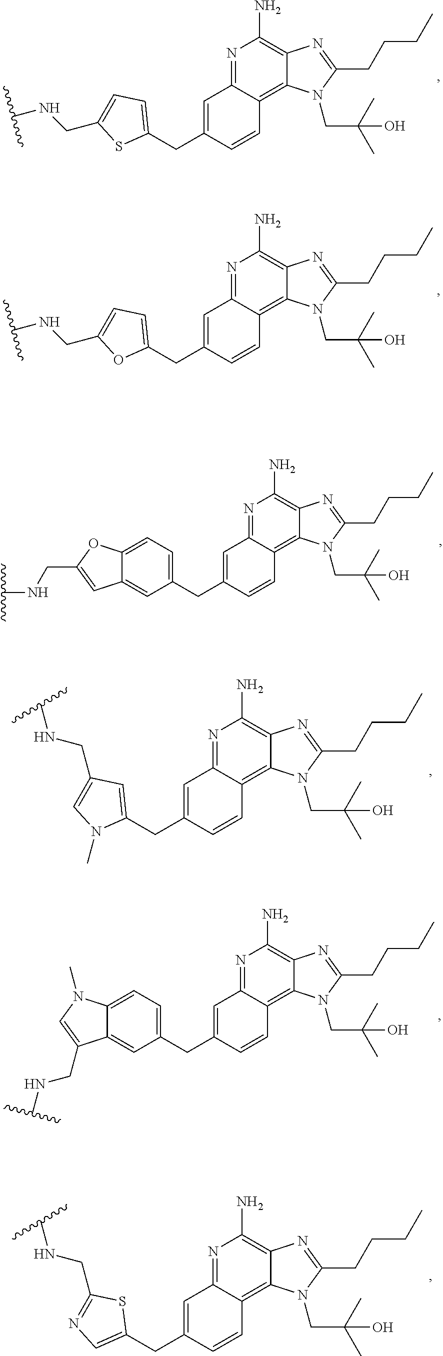 [SMILES: *NCc1ccc(Cc2ccc3c(c2)nc(N)c2nc(CCCC)n(CC(C)(C)O)c23)s1 ; *NCc1ccc(Cc2ccc3c(c2)nc(N)c2nc(CCCC)n(CC(C)(C)O)c23)o1 ; *NCc1cc2cc(Cc3ccc4c(c3)nc(N)c3nc(CCCC)n(CC(C)(C)O)c34)ccc2o1 ; *NCc1cc(Cc2ccc3c(c2)nc(N)c2nc(CCCC)n(CC(C)(C)O)c23)n(C)c1 ; *NCc1cn(C)c2ccc(Cc3ccc4c(c3)nc(N)c3nc(CCCC)n(CC(C)(C)O)c34)cc12 ; *NCc1ncc(Cc2ccc3c(c2)nc(N)c2nc(CCCC)n(CC(C)(C)O)c23)s1]

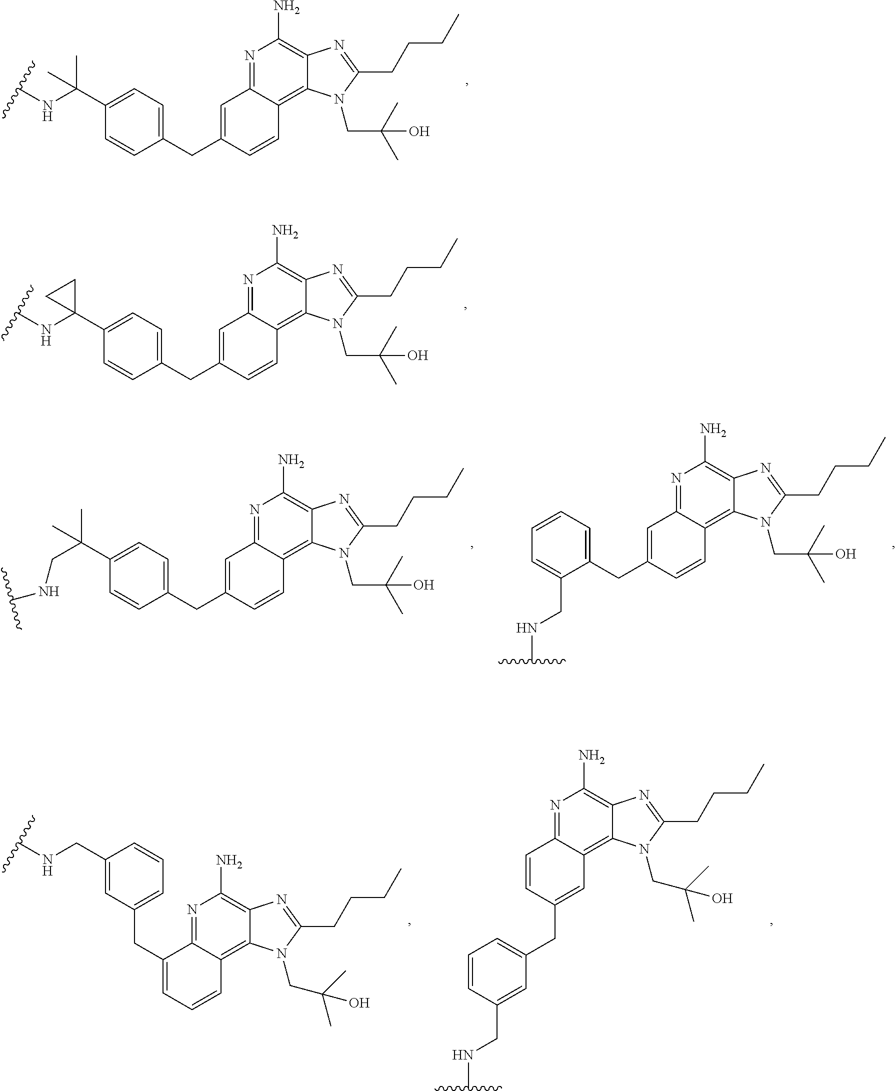 [SMILES: *NC(C)(C)c1ccc(Cc2ccc3c(c2)nc(N)c2nc(CCCC)n(CC(C)(C)O)c23)cc1 ; *NC1(c2ccc(Cc3ccc4c(c3)nc(N)c3nc(CCCC)n(CC(C)(C)O)c34)cc2)CC1 ; *NCC(C)(C)c1ccc(Cc2ccc3c(c2)nc(N)c2nc(CCCC)n(CC(C)(C)O)c23)cc1 ; *NCc1ccccc1Cc1ccc2c(c1)nc(N)c1nc(CCCC)n(CC(C)(C)O)c12 ; *NCc1cccc(Cc2cccc3c2nc(N)c2nc(CCCC)n(CC(C)(C)O)c23)c1 ; *NCc1cccc(Cc2ccc3nc(N)c4nc(CCCC)n(CC(C)(C)O)c4c3c2)c1]

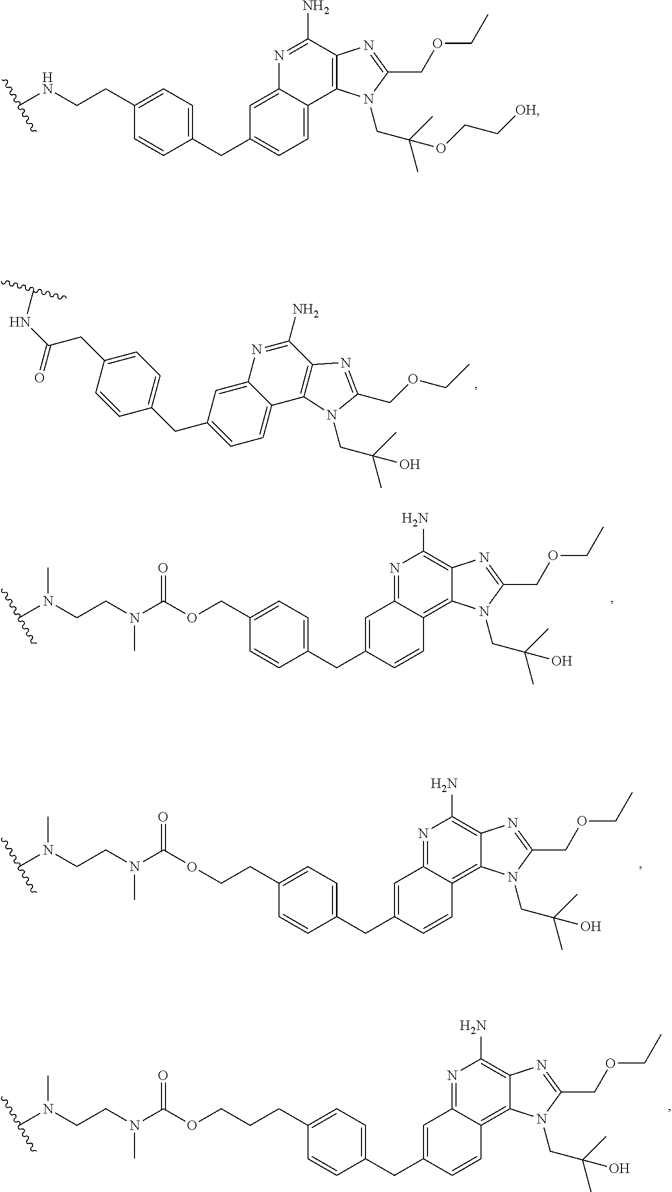 [SMILES: *NCCc1ccc(Cc2ccc3c(c2)nc(N)c2nc(COCC)n(CC(C)(C)OCCO)c23)cc1 ; *NC(=O)Cc1ccc(Cc2ccc3c(c2)nc(N)c2nc(COCC)n(CC(C)(C)O)c23)cc1 ; *N(C)CCN(C)C(=O)OCc1ccc(Cc2ccc3c(c2)nc(N)c2nc(COCC)n(CC(C)(C)O)c23)cc1 ; *N(C)CCN(C)C(=O)OCCc1ccc(Cc2ccc3c(c2)nc(N)c2nc(COCC)n(CC(C)(C)O)c23)cc1 ; *N(C)CCN(C)C(=O)OCCCc1ccc(Cc2ccc3c(c2)nc(N)c2nc(COCC)n(CC(C)(C)O)c23)cc1]

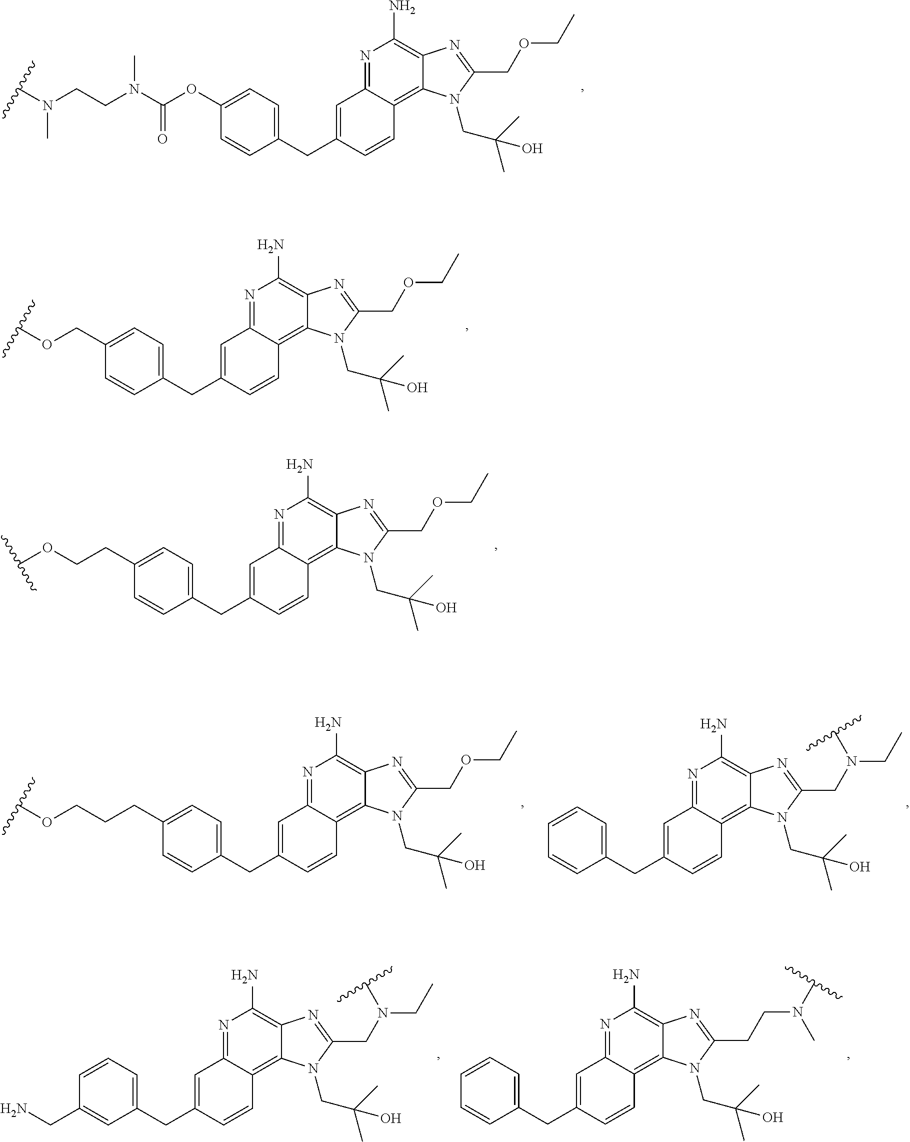

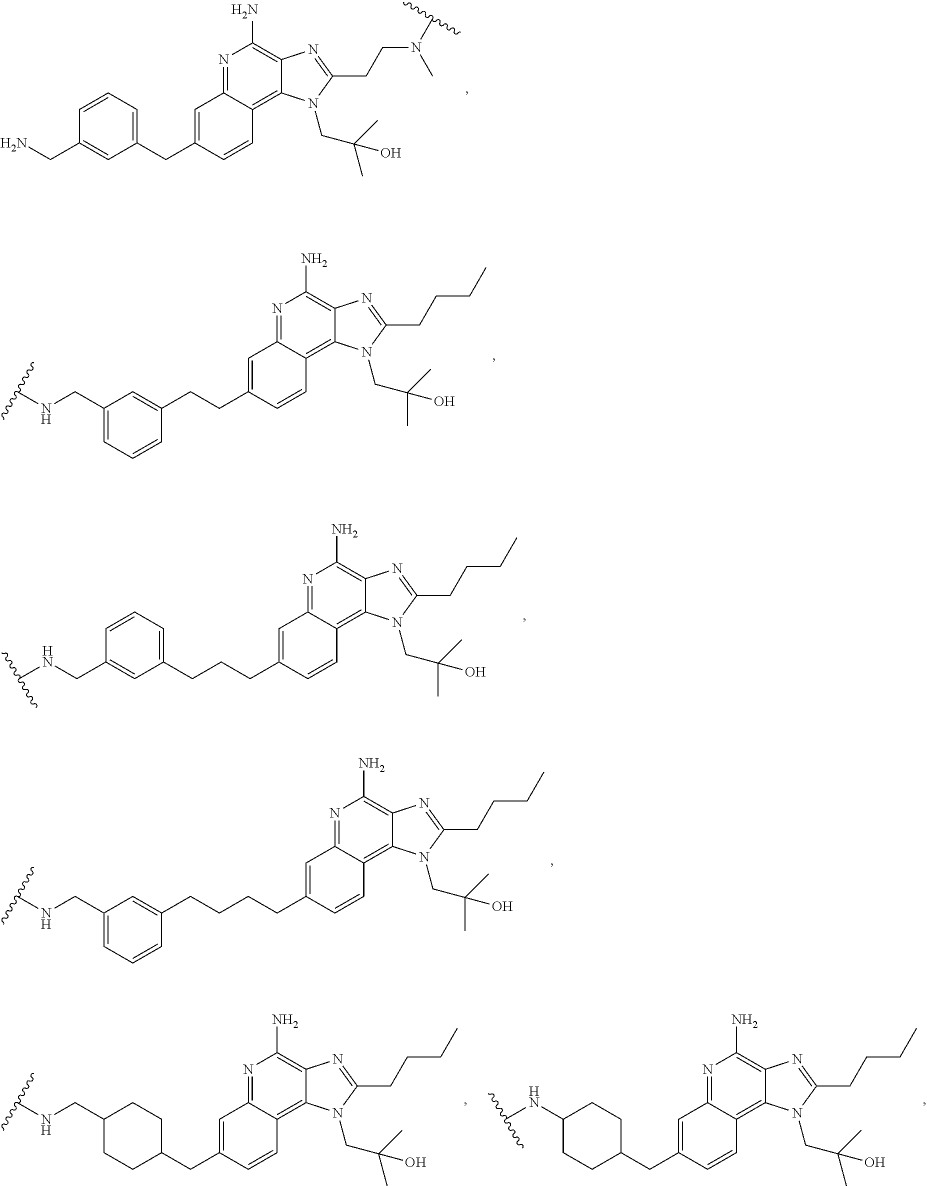 [SMILES: *N(C)CCc1nc2c(N)nc3cc(Cc4cccc(CN)c4)ccc3c2n1CC(C)(C)O ; *NCc1cccc(CCc2ccc3c(c2)nc(N)c2nc(CCCC)n(CC(C)(C)O)c23)c1 ; *NCc1cccc(CCCc2ccc3c(c2)nc(N)c2nc(CCCC)n(CC(C)(C)O)c23)c1 ; *NCc1cccc(CCCCc2ccc3c(c2)nc(N)c2nc(CCCC)n(CC(C)(C)O)c23)c1 ; *NCC1CCC(Cc2ccc3c(c2)nc(N)c2nc(CCCC)n(CC(C)(C)O)c23)CC1 ; *NC1CCC(Cc2ccc3c(c2)nc(N)c2nc(CCCC)n(CC(C)(C)O)c23)CC1]

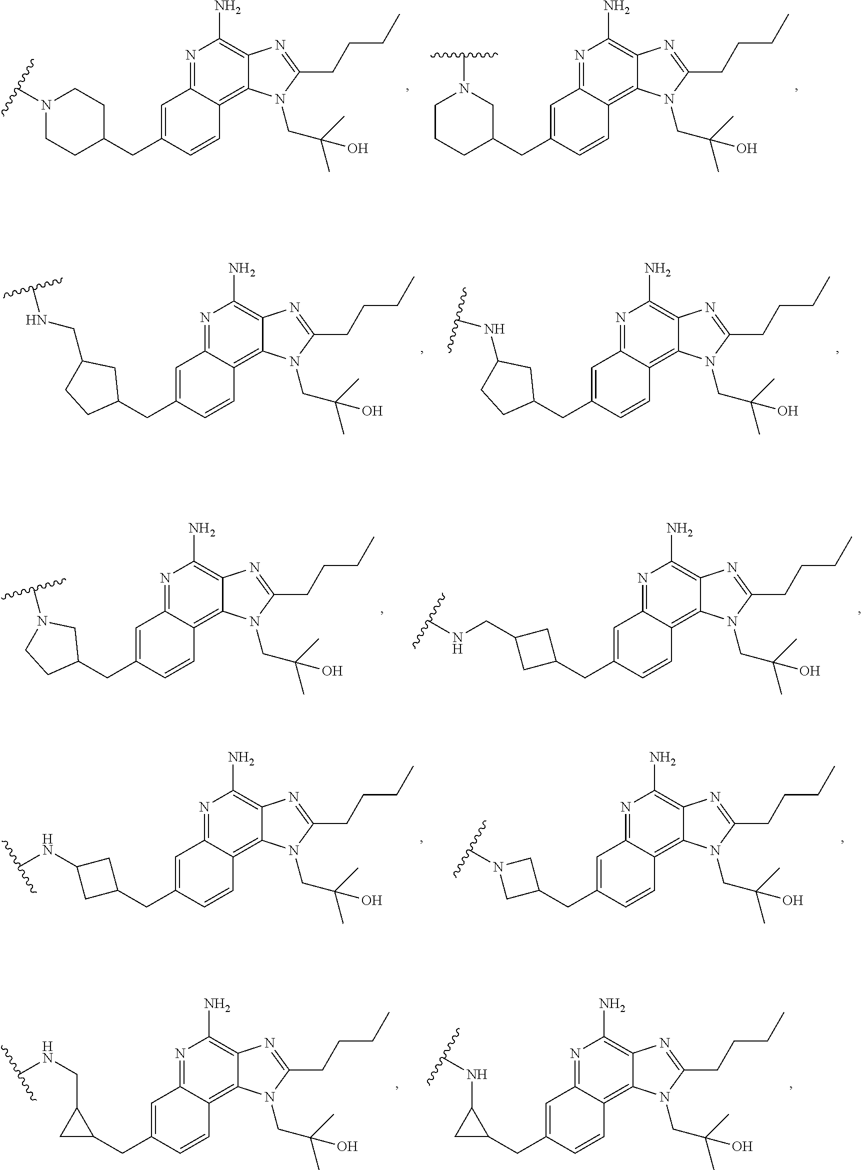

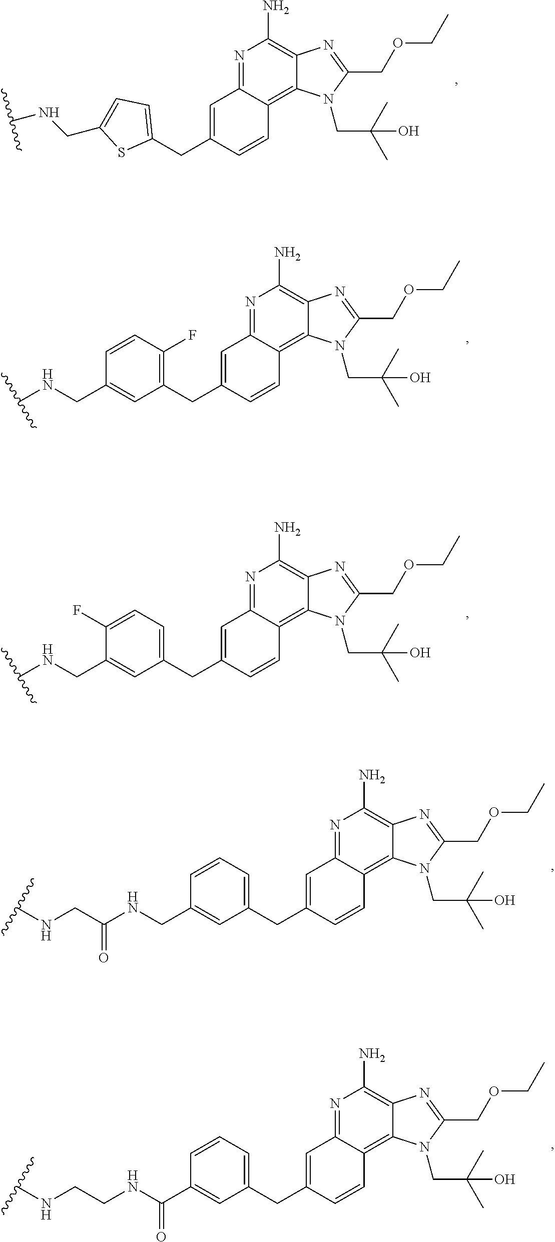 [SMILES: *NCc1ccc(Cc2ccc3c(c2)nc(N)c2nc(COCC)n(CC(C)(C)O)c23)s1 ; *NCc1ccc(F)c(Cc2ccc3c(c2)nc(N)c2nc(COCC)n(CC(C)(C)O)c23)c1 ; *NCc1cc(Cc2ccc3c(c2)nc(N)c2nc(COCC)n(CC(C)(C)O)c23)ccc1F ; *NCC(=O)NCc1cccc(Cc2ccc3c(c2)nc(N)c2nc(COCC)n(CC(C)(C)O)c23)c1 ; *NCCNC(=O)c1cccc(Cc2ccc3c(c2)nc(N)c2nc(COCC)n(CC(C)(C)O)c23)c1]

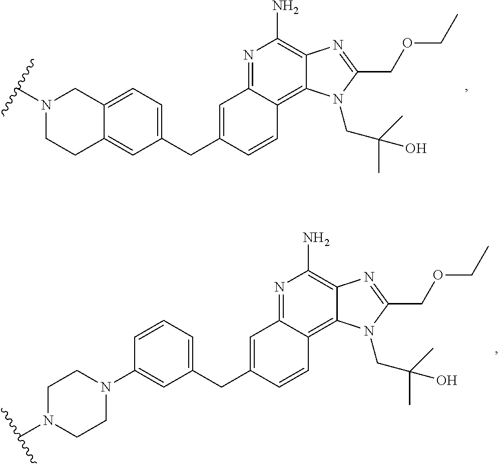 [SMILES: *N1CCc2cc(Cc3ccc4c(c3)nc(N)c3nc(COCC)n(CC(C)(C)O)c34)ccc2C1 ; *N1CCN(c2cccc(Cc3ccc4c(c3)nc(N)c3nc(COCC)n(CC(C)(C)O)c34)c2)CC1]

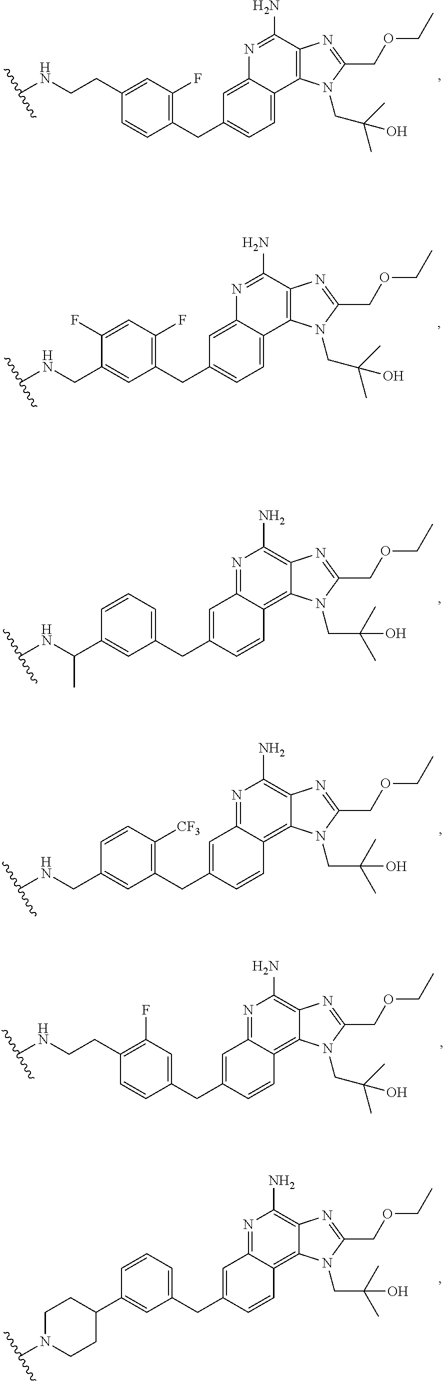 [SMILES: *NCCc1ccc(Cc2ccc3c(c2)nc(N)c2nc(COCC)n(CC(C)(C)O)c23)c(F)c1 ; *NCc1cc(Cc2ccc3c(c2)nc(N)c2nc(COCC)n(CC(C)(C)O)c23)c(F)cc1F ; *NC(C)c1cccc(Cc2ccc3c(c2)nc(N)c2nc(COCC)n(CC(C)(C)O)c23)c1 ; *NCc1ccc(C(F)(F)F)c(Cc2ccc3c(c2)nc(N)c2nc(COCC)n(CC(C)(C)O)c23)c1 ; *NCCc1ccc(Cc2ccc3c(c2)nc(N)c2nc(COCC)n(CC(C)(C)O)c23)cc1F ; *N1CCC(c2cccc(Cc3ccc4c(c3)nc(N)c3nc(COCC)n(CC(C)(C)O)c34)c2)CC1]

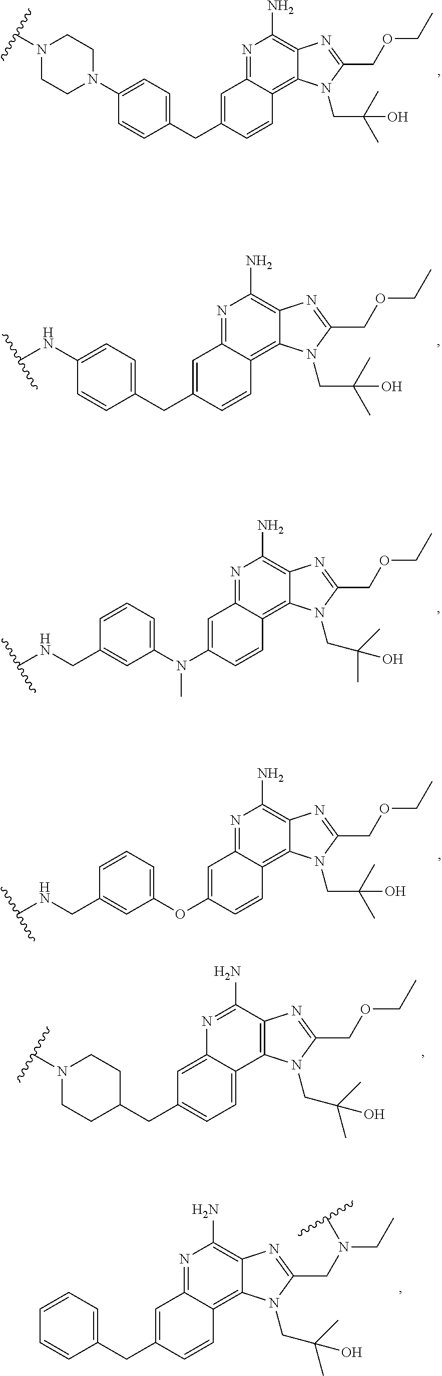 [SMILES: *N1CCN(c2ccc(Cc3ccc4c(c3)nc(N)c3nc(COCC)n(CC(C)(C)O)c34)cc2)CC1 ; *Nc1ccc(Cc2ccc3c(c2)nc(N)c2nc(COCC)n(CC(C)(C)O)c23)cc1 ; *NCc1cccc(N(C)c2ccc3c(c2)nc(N)c2nc(COCC)n(CC(C)(C)O)c23)c1 ; *NCc1cccc(Oc2ccc3c(c2)nc(N)c2nc(COCC)n(CC(C)(C)O)c23)c1 ; *N1CCC(Cc2ccc3c(c2)nc(N)c2nc(COCC)n(CC(C)(C)O)c23)CC1 ; *N(CC)Cc1nc2c(N)nc3cc(Cc4ccccc4)ccc3c2n1CC(C)(C)O]

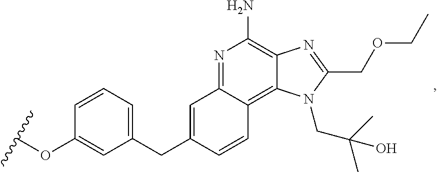 [SMILES: *Oc1cccc(Cc2ccc3c(c2)nc(N)c2nc(COCC)n(CC(C)(C)O)c23)c1]

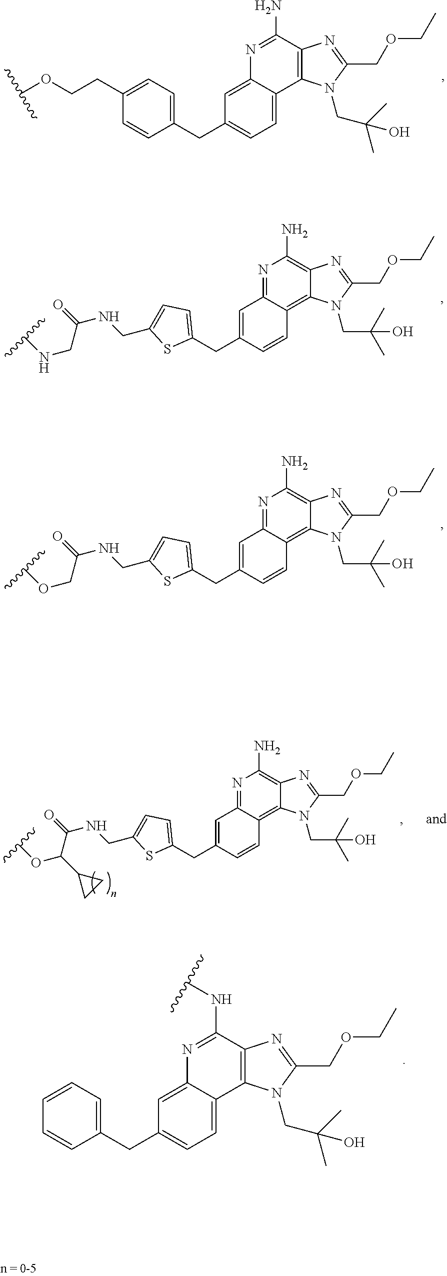

In some embodiments, L′ has the Formula (IIIa):

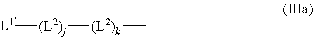

wherein
L^{1′} is a stretcher unit precursor comprising a reactive group RG capable of reacting with the targeting moiety to form a stretcher unit L¹ covalently attached to the targeting moiety, wherein the reactive group RG is selected from the group consisting of maleimide, thiol, amino, bromide, bromoacetamido, iodoacetamido, p-toluenesulfonate, iodide, hydroxyl, carboxyl, pyridyl disulfide, and N-hydroxysuccinimide;
L² is an optional peptide unit of two to twelve amino acid residues;
L³ is an optional spacer unit covalently attached to a payload unit, and
j and k are independently selected from 0 and 1.

In some embodiments, L^{1′} has the formula is selected from the group consisting of:

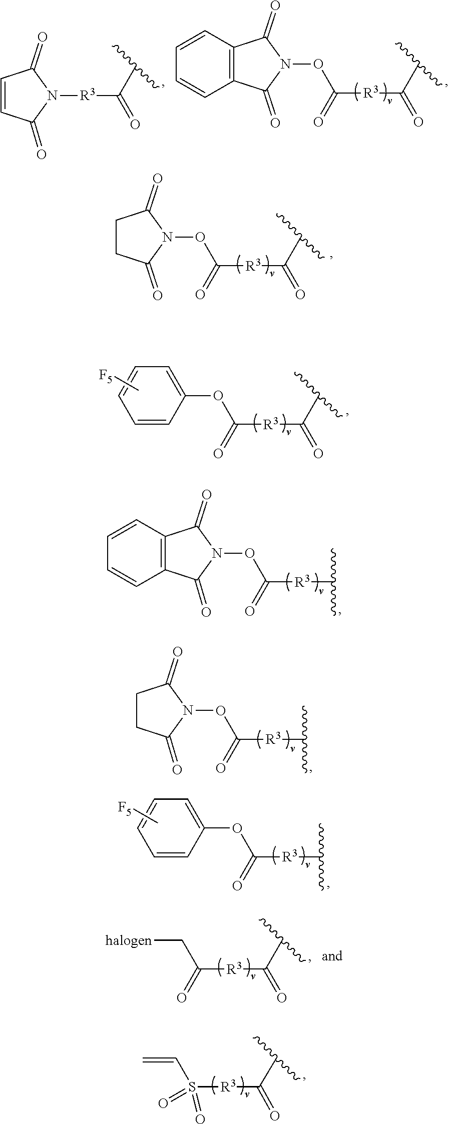

wherein each R³ is independently selected from the group consisting of bond, alkyl, heteroalkyl, cycloalkyl, heterocyclyl, aryl, heteroaryl, alkylaryl, arylalkyl, alkylcycloalkyl, cycloalkylalkyl, alkylheterocyclyl, heterocyclylalkyl, -alkyl-C(O)N(R^{a})-alkyl-N(R^{a}), —N(R^{a})-alkyl-, and —(CH₂CH₂O)ᵣ—CH₂—, wherein R^{a} is H or alkyl, and r is an integer ranging from 1 to 10, v is an integer ranging from 0 to 5.

In certain embodiments, v is 1 and R³ is (CH₂)₅.

In some embodiments, j is 0 and k is 0.

In some embodiments, j is 0 and k is 1.

In some embodiments, L^{1′} has the formula:

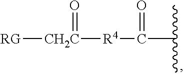

wherein R⁴ is selected from the group consisting of alkyl, -alkyl-O—, —N(R^{a})-alkyl-N(R^{a})—, —N(R^{a})-alkyl-, and (CH₂CH₂O)ᵣ—CH₂; wherein R^{a} is H or alkyl, and r is an integer ranging from 1 to 10.

In some embodiments, L^{1′} has the formula:

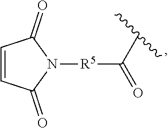

wherein R⁵ is selected from alkyl, -alkyl-O—, aryl, —N(R^{a})-alkyl- or —(CH₂CH₂O), —CH₂—; wherein R^{a} is H or alkyl, and r is an integer ranging from 1 to 10. In certain embodiments, R⁵ is selected from the group consisting of C₁₋₁₀ alkyl, —(C₁₋₁₀ alkyl)-O—, —N(R^{a})—(C₂₋₆ alkyl)— or —(CH₂CH₂O)ᵣ—CH₂—; wherein R^{a} is H or C₁₋₆ alkyl, and r is an integer ranging from 1 to 10.

In some embodiments, L′ reacts with a cysteine amino acid of the targeting moiety to form a thioether bond, and R⁵ is —(C₂₋₆ alkyl)-O—, wherein the C₂₋₆ alkyl is optionally substituted with F, OH, O(C₁₋₆ alkyl), NH₂, NHCH₃, N(CH₃)₂, OP(O)₃H₂, and C₁₋₆ alkyl, wherein the C₁₋₆ alkyl is optionally substituted with one or more F.

In certain embodiments, L′ reacts with a lysine amino acid of the targeting moiety to form an amide bond, and R⁵ is —(C₂₋₆ alkyl)-O—, wherein the C₂₋₆ alkyl is optionally substituted with F, OH, O(C₁₋₆ alkyl), NH₂, NHCH₃, N(CH₃)₂, OP(O)₃H₂, and C₁₋₆ alkyl, wherein the C₁₋₆ alkyl is optionally substituted with one or more F.

In some embodiments, j is 1 and k is 1.

In some embodiments, j is 1 and L² comprises two or twelve amino acid residues independently selected from glycine, alanine, phenylalanine, lysine, arginine, valine, and citrylline.

In certain embodiments, L² is valine-citrylline.

In some embodiments, k is 1 and L³ comprises para-aminobenzyl or para-aminobenzyloxycarbonyl.

In certain embodiments, there is provided a linker-payload compound having a formula:

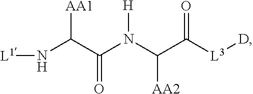

wherein AA1 and AA2 are independently selected from an amino acid side chain; p is an integer from 1 to 8.

In certain embodiments, the amino acid side chain is independently selected from H, —CH₃, —CH₂(C₆H₅), —CH₂CH₂CH₂CH₂NH₂, —CH₂CH₂CH₂NHC(NH)NH₂, —CHCH(CH₃)CH₃, and —CH₂CH₂CH₂NHC(O)NH₂.

In certain embodiments, there is provided a linker-payload compound having a formula:

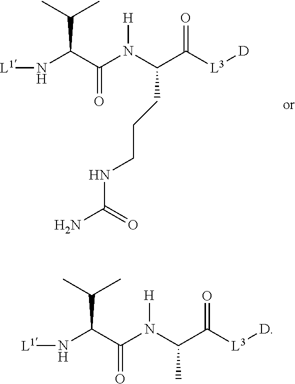

In certain embodiments, there is provided a linker-payload compound having a formula:

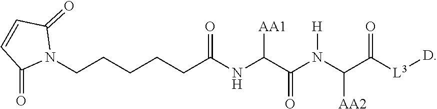

In certain embodiments, there is provided a linker-payload compound having a formula:

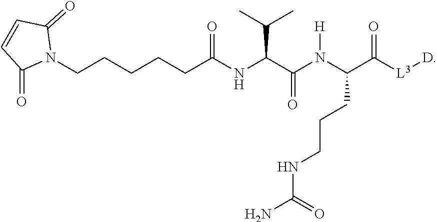

In certain embodiments, there is provided a linker-payload compound having a formula:

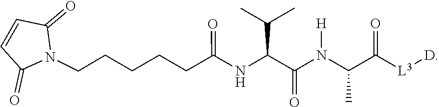

In certain embodiments, there is provided a linker-payload compound having a formula:

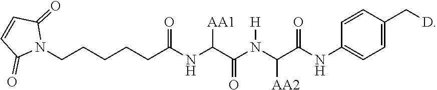

In certain embodiments, there is provided a linker-payload compound having a formula:

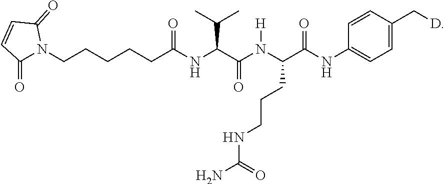

In certain embodiments, there is provided a linker-payload compound having a formula:

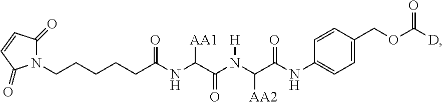

In certain embodiments, there is provided a linker-payload compound having a formula:

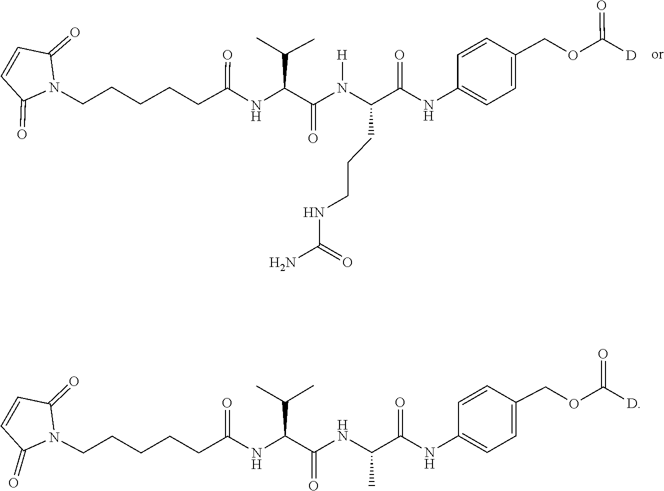

In some embodiments, there is provided a linker-payload compound having a formula selected from the group consisting of:

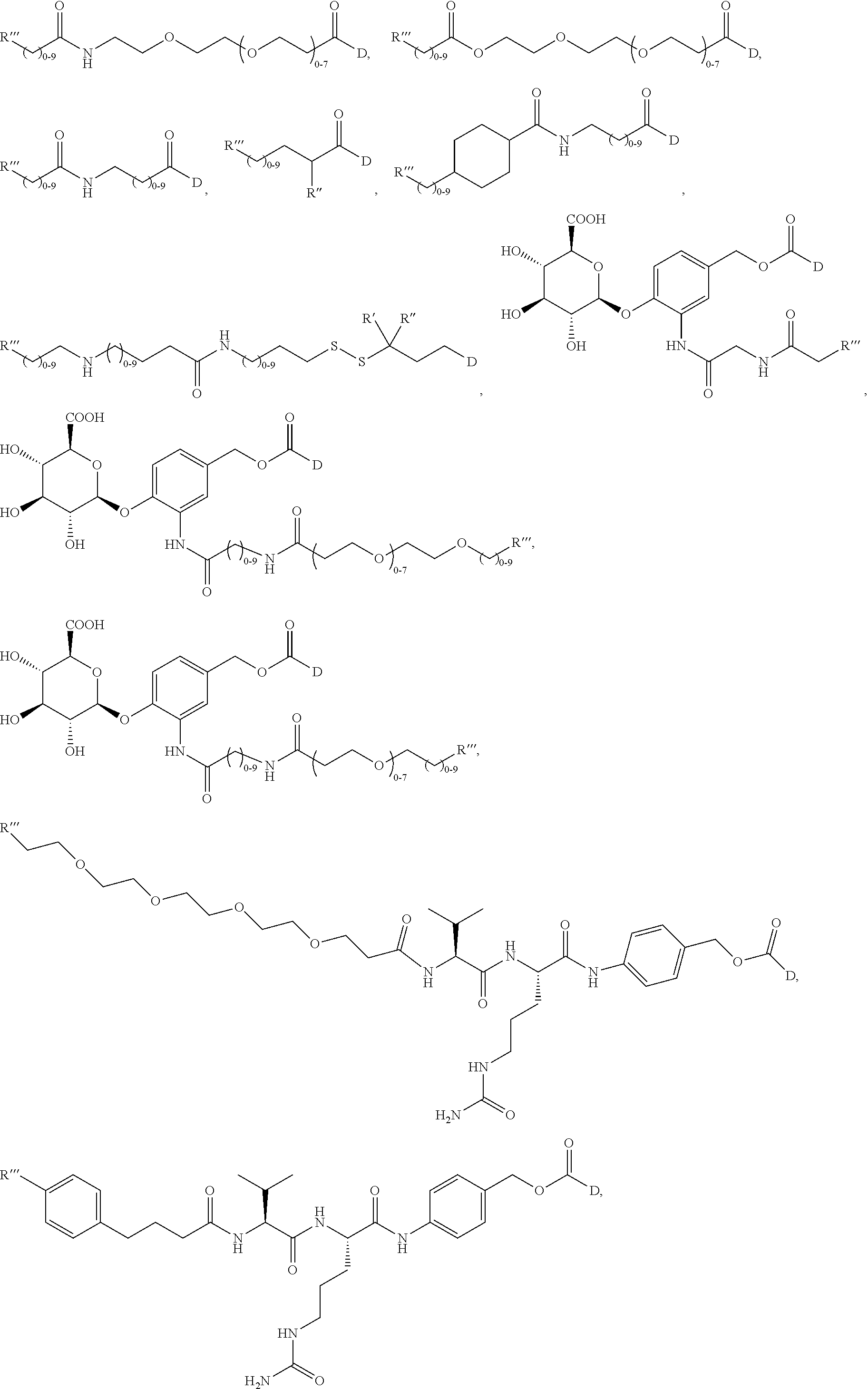

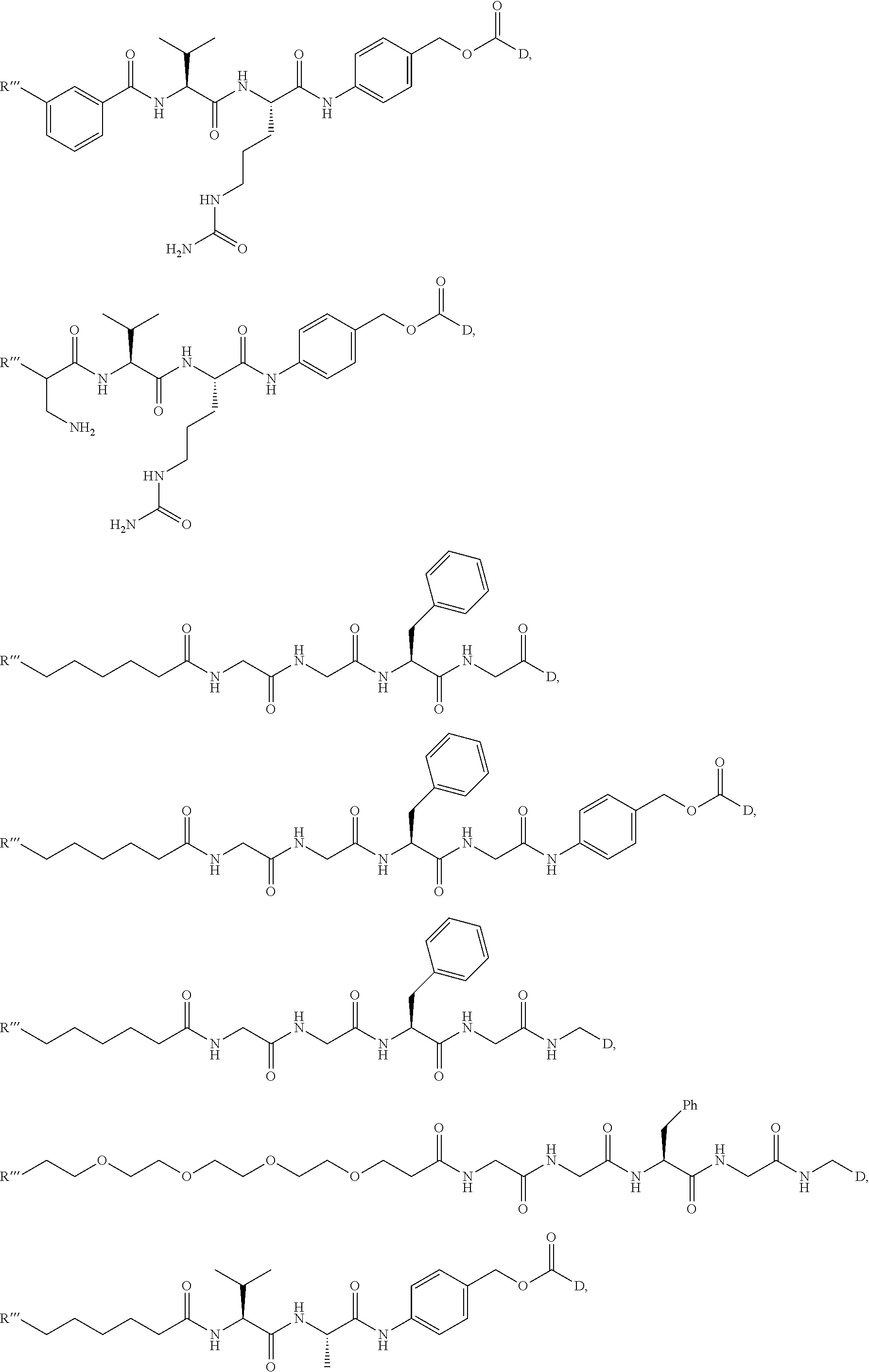

wherein R′″ is selected from the group consisting of:

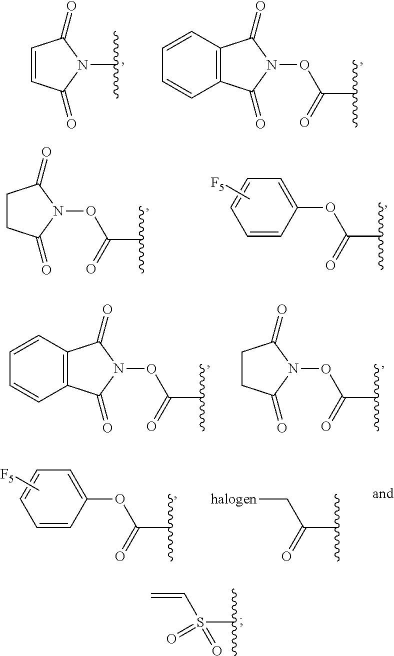

wherein R′ and R″ are independently a bond, hydrogen or methyl.

In some embodiments, there is provided a linker-payload compound having a formula selected from the group consisting of:

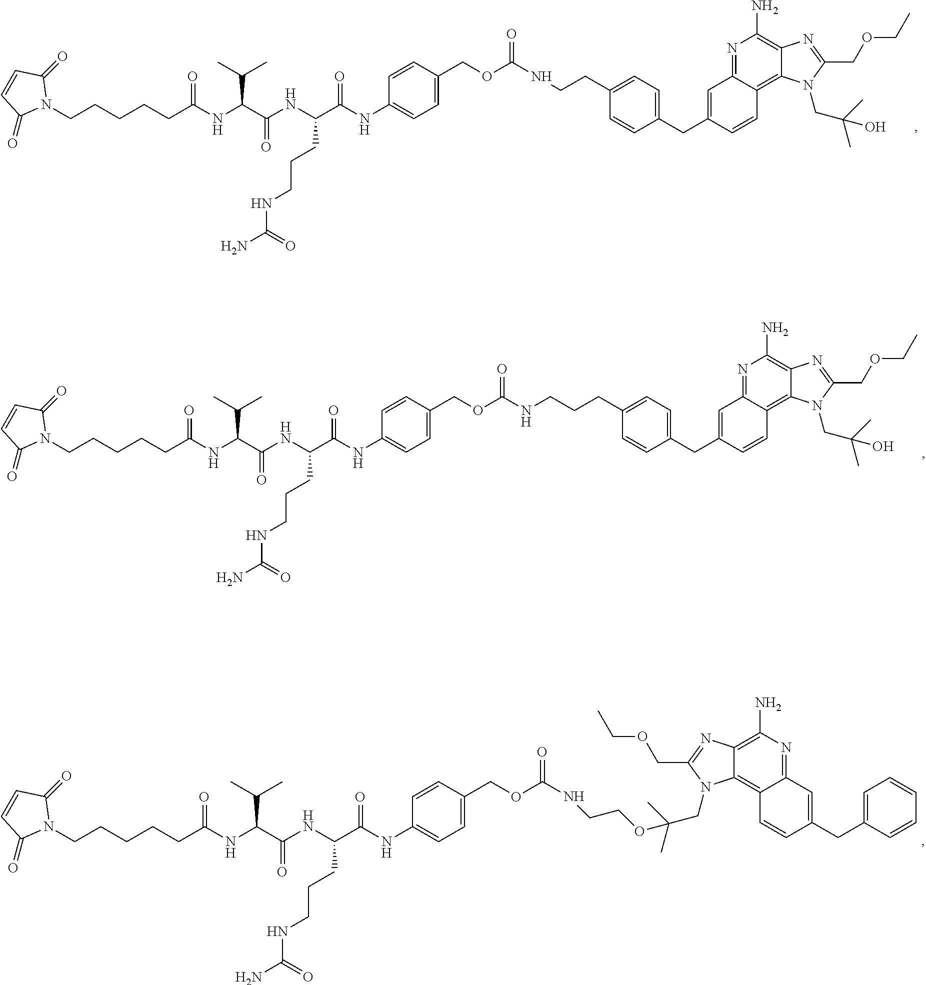 [SMILES: CCOCc1nc2c(N)nc3cc(Cc4ccc(CCNC(=O)OCc5ccc(NC(=O)[C@H](CCCNC(N)=O)NC(=O)[C@@H](NC(=O)CCCCCN6C(=O)C=CC6=O)C(C)C)cc5)cc4)ccc3c2n1CC(C)(C)O ; CCOCc1nc2c(N)nc3cc(Cc4ccc(CCCNC(=O)OCc5ccc(NC(=O)[C@H](CCCNC(N)=O)NC(=O)[C@@H](NC(=O)CCCCCN6C(=O)C=CC6=O)C(C)C)cc5)cc4)ccc3c2n1CC(C)(C)O ; CCOCc1nc2c(N)nc3cc(Cc4ccccc4)ccc3c2n1CC(C)(C)OCCNC(=O)OCc1ccc(NC(=O)[C@H](CCCNC(N)=O)NC(=O)[C@@H](NC(=O)CCCCCN2C(=O)C=CC2=O)C(C)C)cc1]

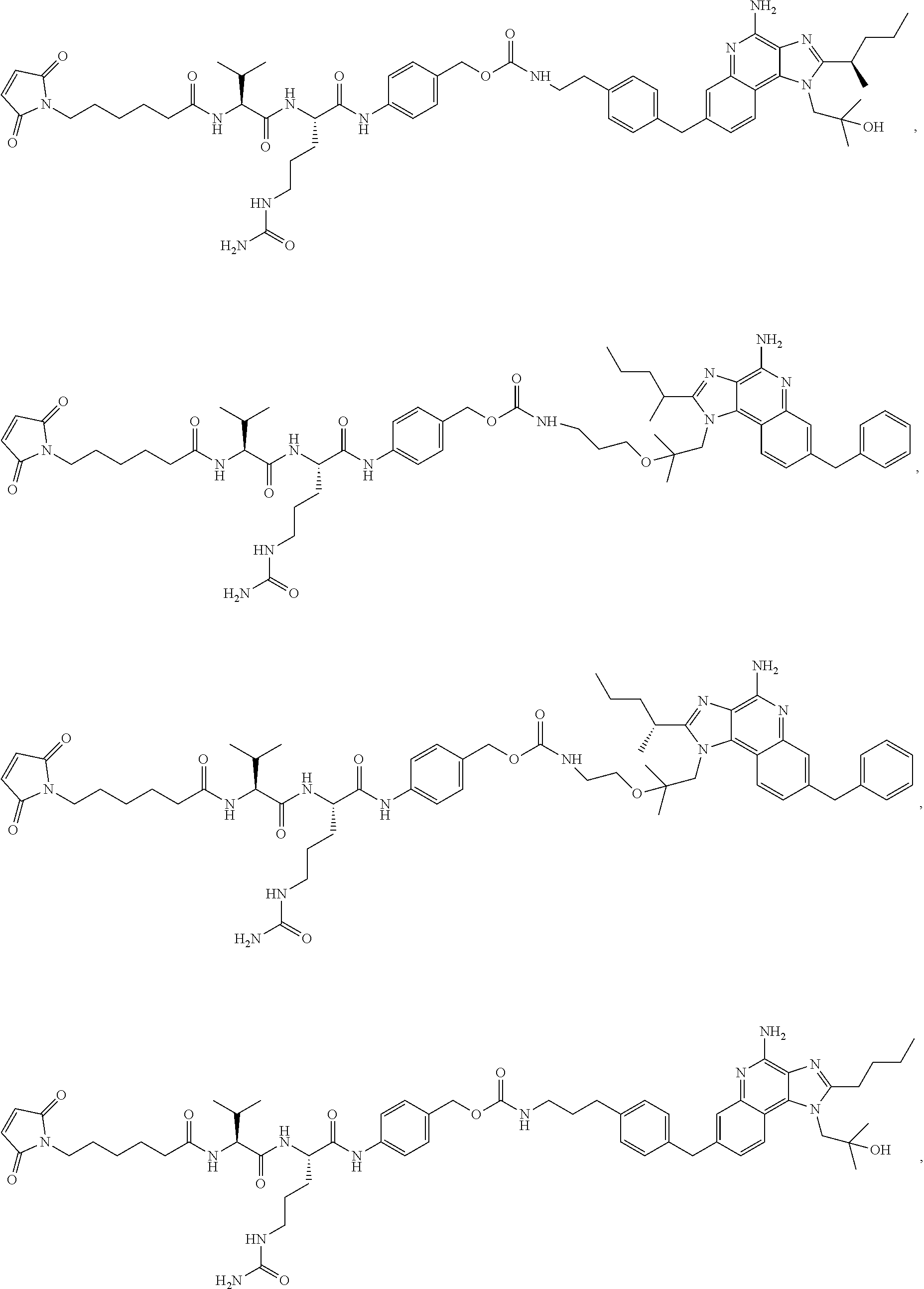

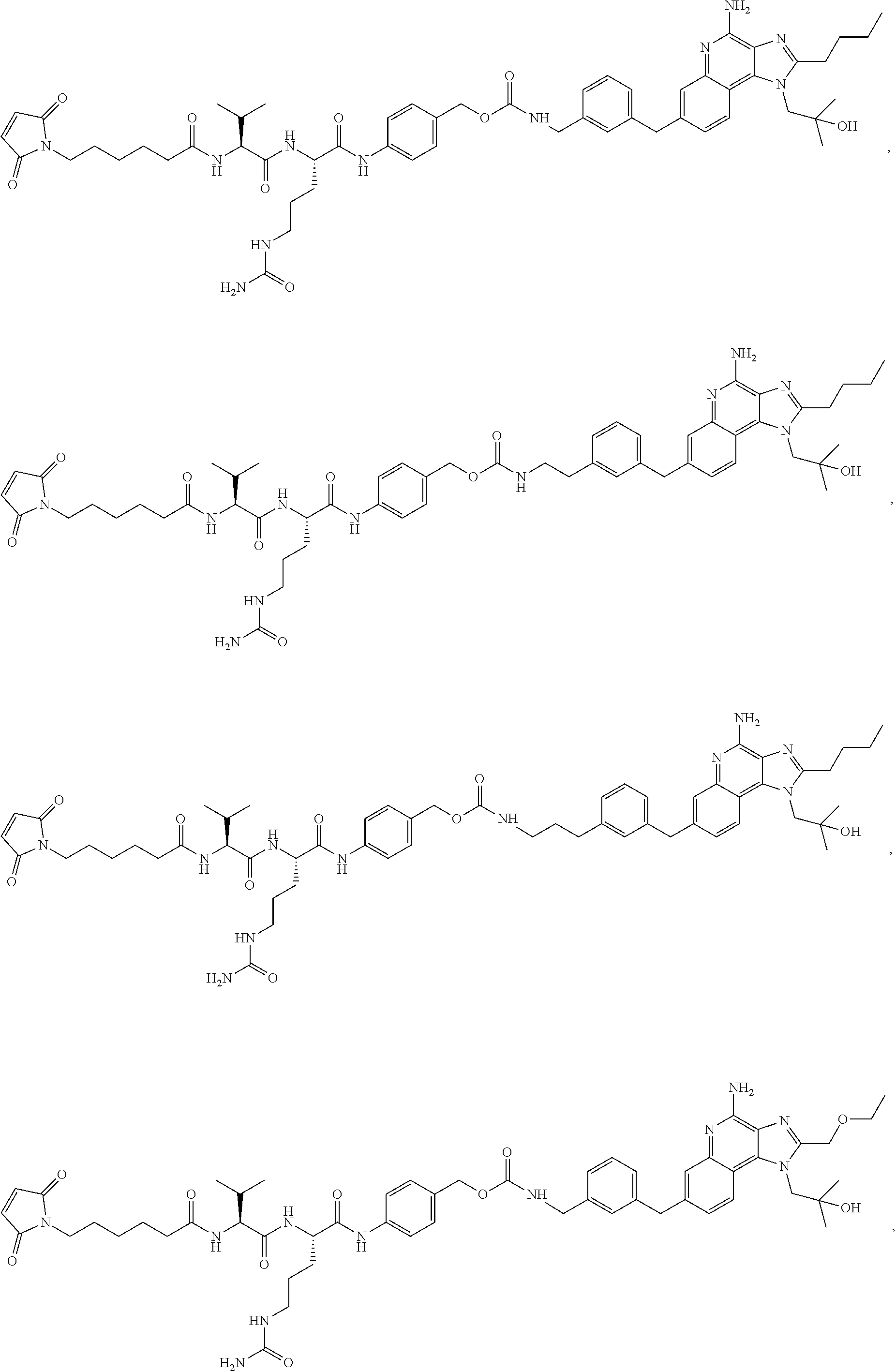

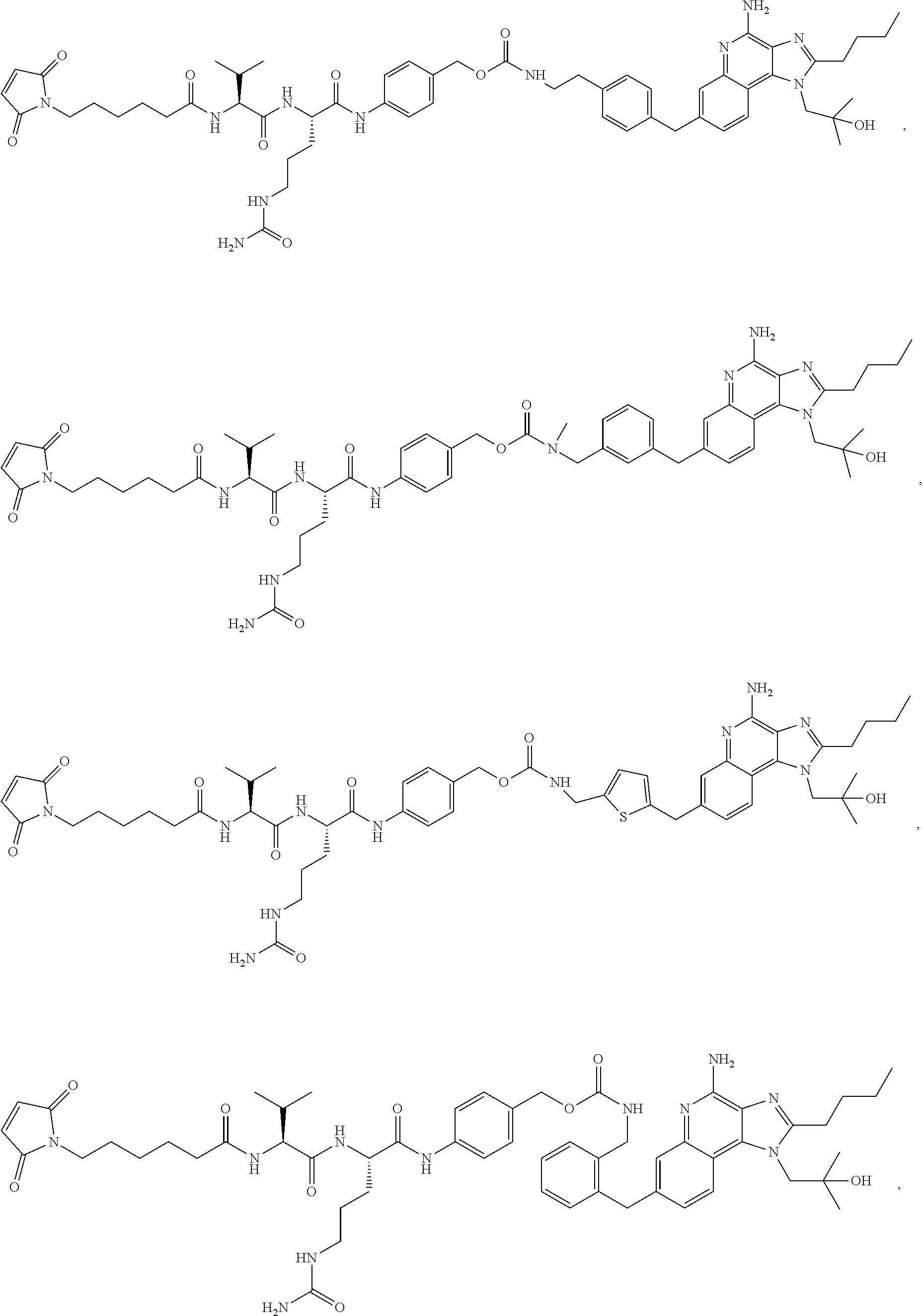

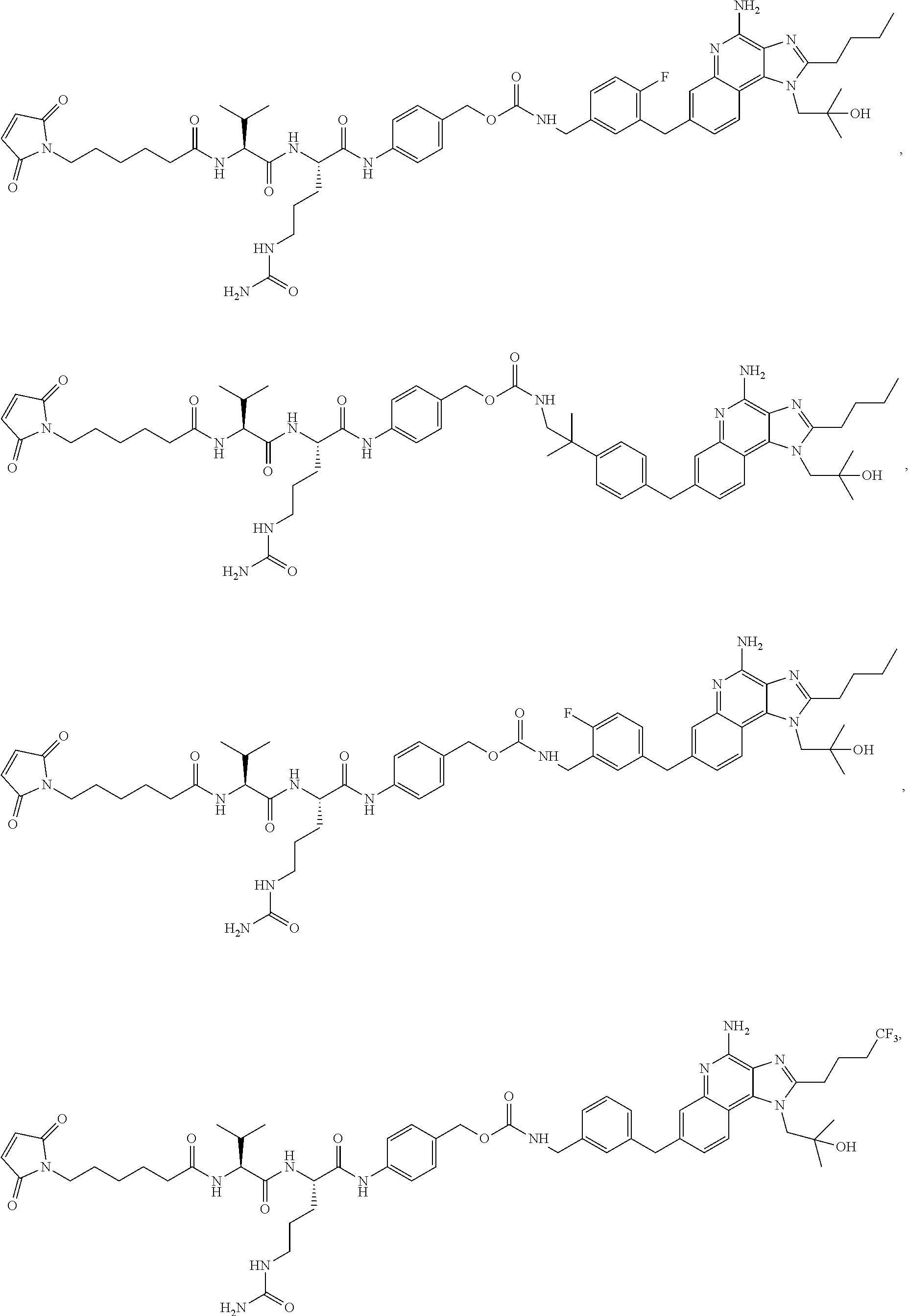

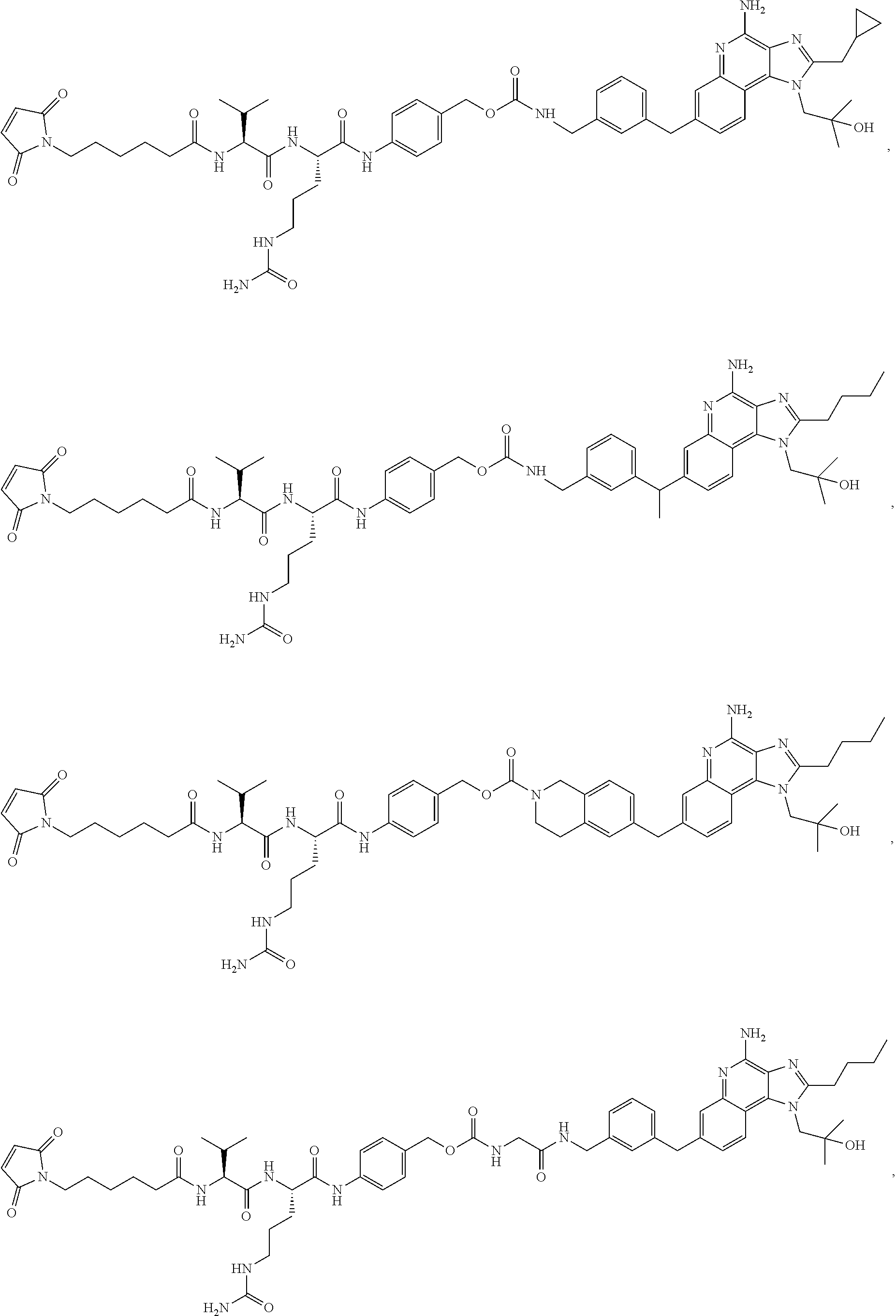

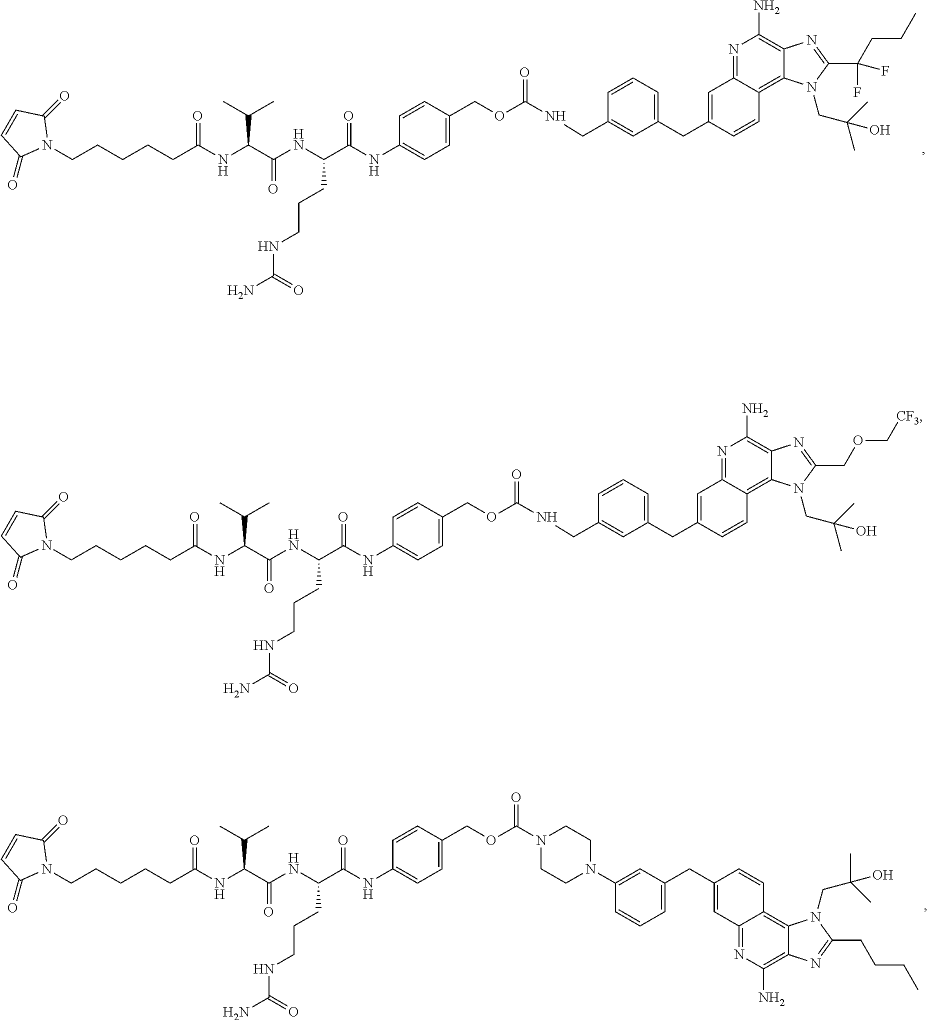 [SMILES: CCCC(F)(F)c1nc2c(N)nc3cc(Cc4cccc(CNC(=O)OCc5ccc(NC(=O)[C@H](CCCNC(N)=O)NC(=O)[C@@H](NC(=O)CCCCCN6C(=O)C=CC6=O)C(C)C)cc5)c4)ccc3c2n1CC(C)(C)O ; CC(C)[C@H](NC(=O)CCCCCN1C(=O)C=CC1=O)C(=O)N[C@@H](CCCNC(N)=O)C(=O)Nc1ccc(COC(=O)NCc2cccc(Cc3ccc4c(c3)nc(N)c3nc(COCC(F)(F)F)n(CC(C)(C)O)c34)c2)cc1 ; CCCCc1nc2c(N)nc3cc(Cc4cccc(N5CCN(C(=O)OCc6ccc(NC(=O)[C@H](CCCNC(N)=O)NC(=O)[C@@H](NC(=O)CCCCCN7C(=O)C=CC7=O)C(C)C)cc6)CC5)c4)ccc3c2n1CC(C)(C)O]

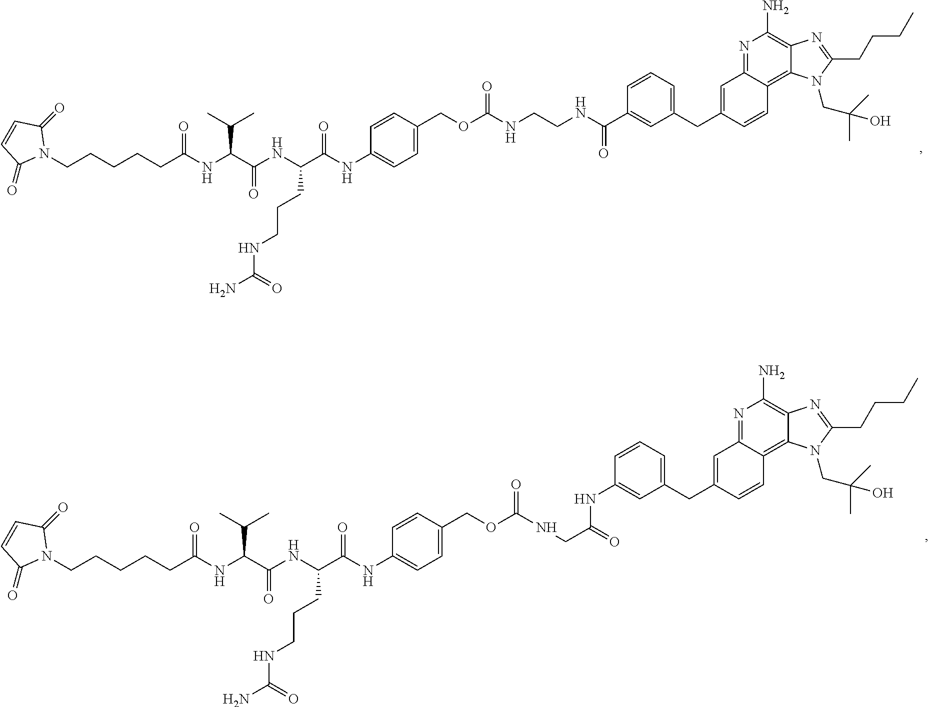 [SMILES: CCCCc1nc2c(N)nc3cc(Cc4cccc(C(=O)NCCNC(=O)OCc5ccc(NC(=O)[C@H](CCCNC(N)=O)NC(=O)[C@@H](NC(=O)CCCCCN6C(=O)C=CC6=O)C(C)C)cc5)c4)ccc3c2n1CC(C)(C)O ; CCCCc1nc2c(N)nc3cc(Cc4cccc(NC(=O)CNC(=O)OCc5ccc(NC(=O)[C@H](CCCNC(N)=O)NC(=O)[C@@H](NC(=O)CCCCCN6C(=O)C=CC6=O)C(C)C)cc5)c4)ccc3c2n1CC(C)(C)O]

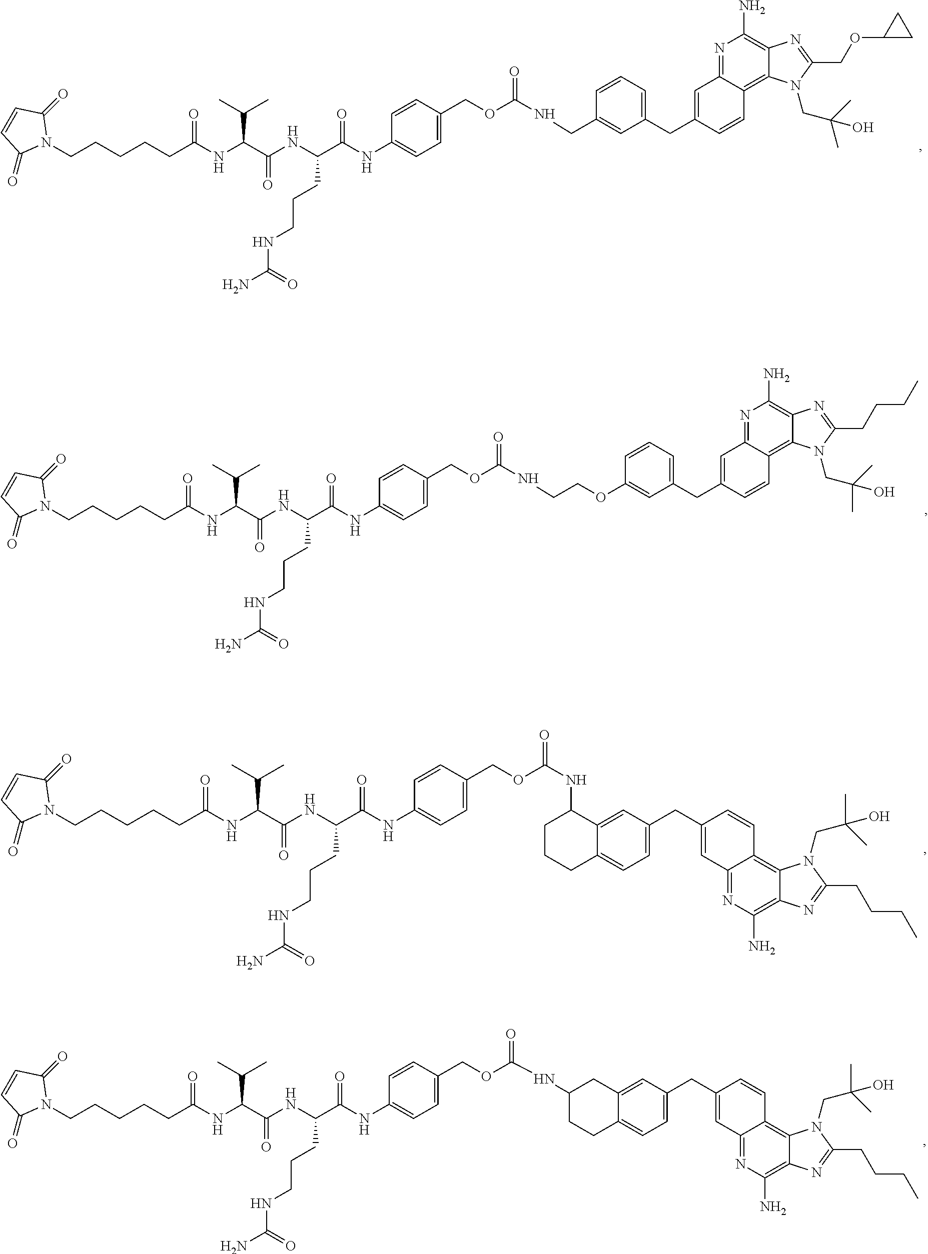

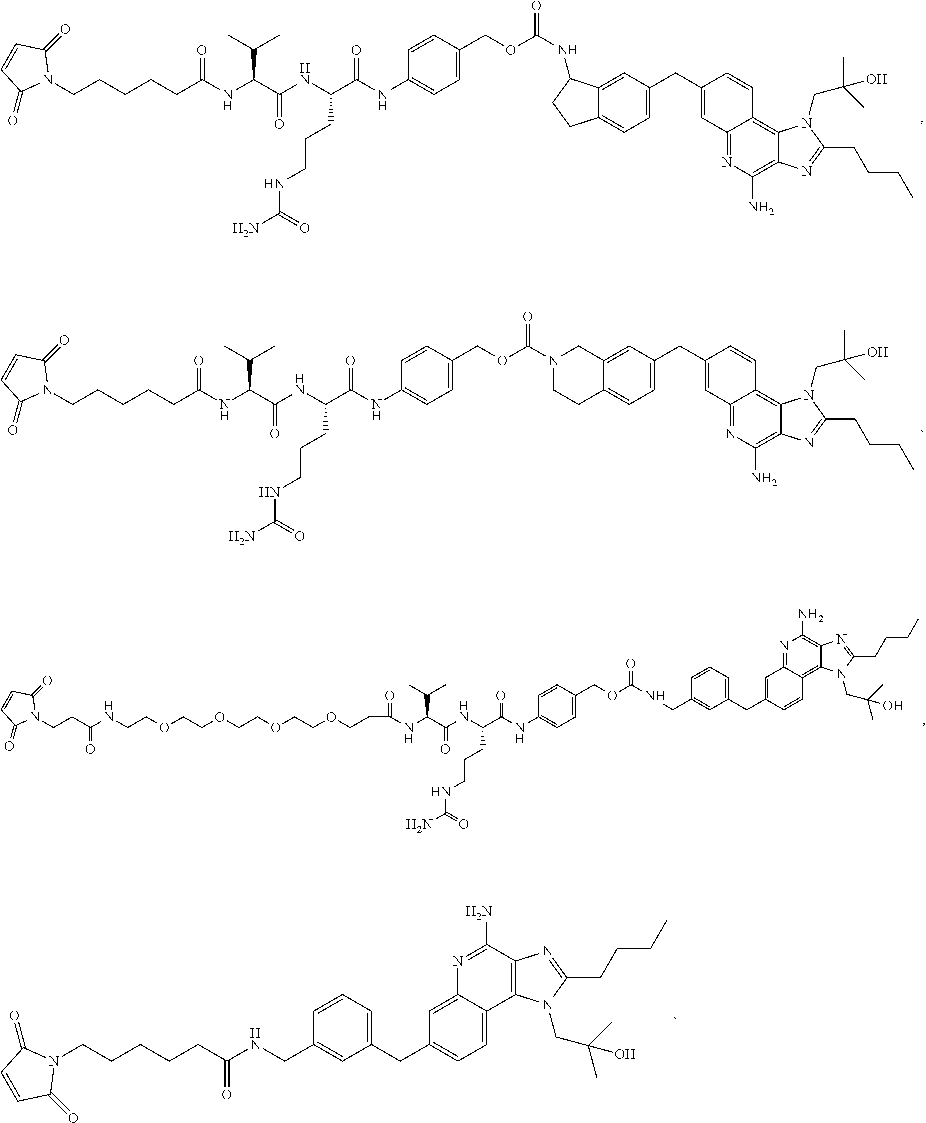

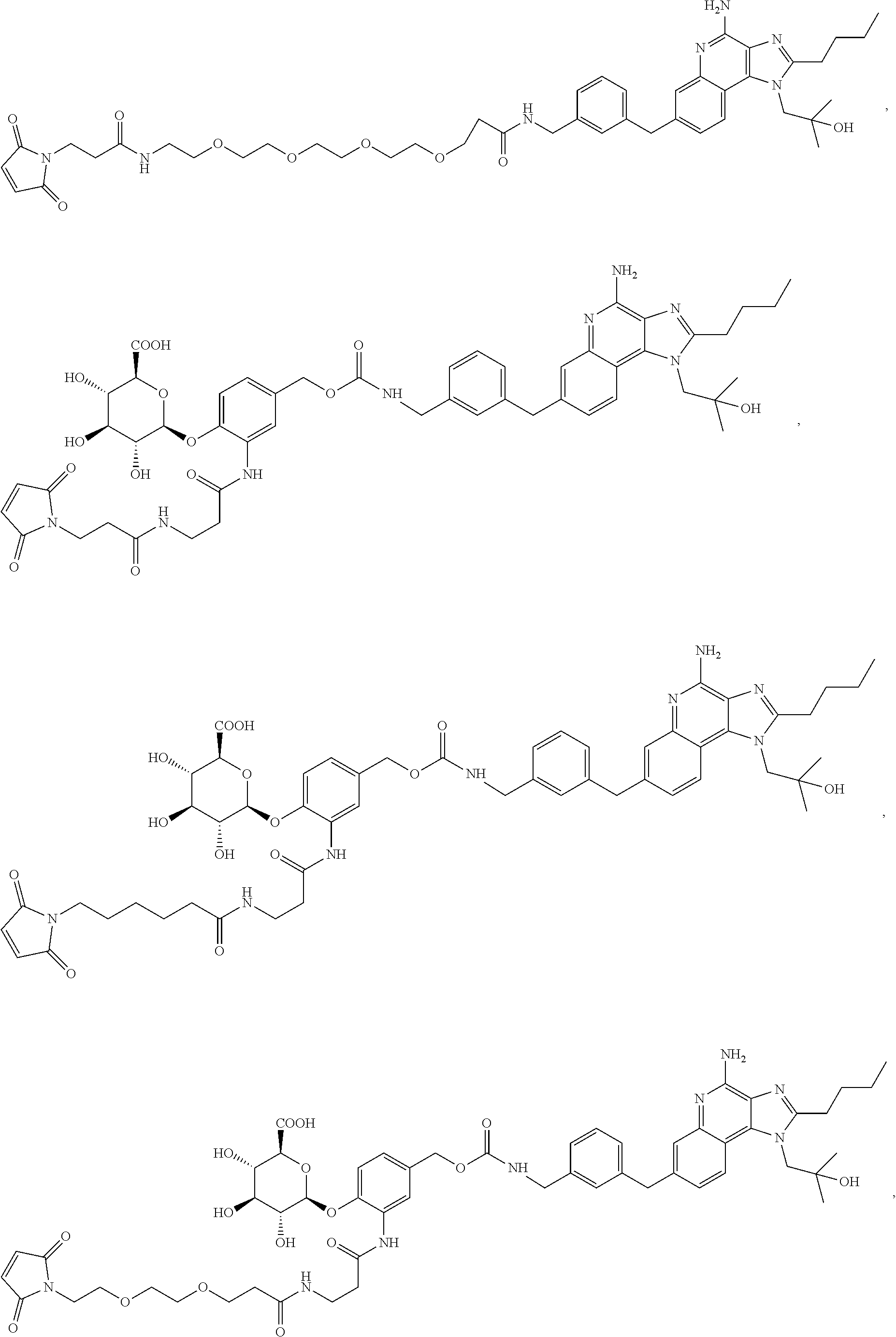

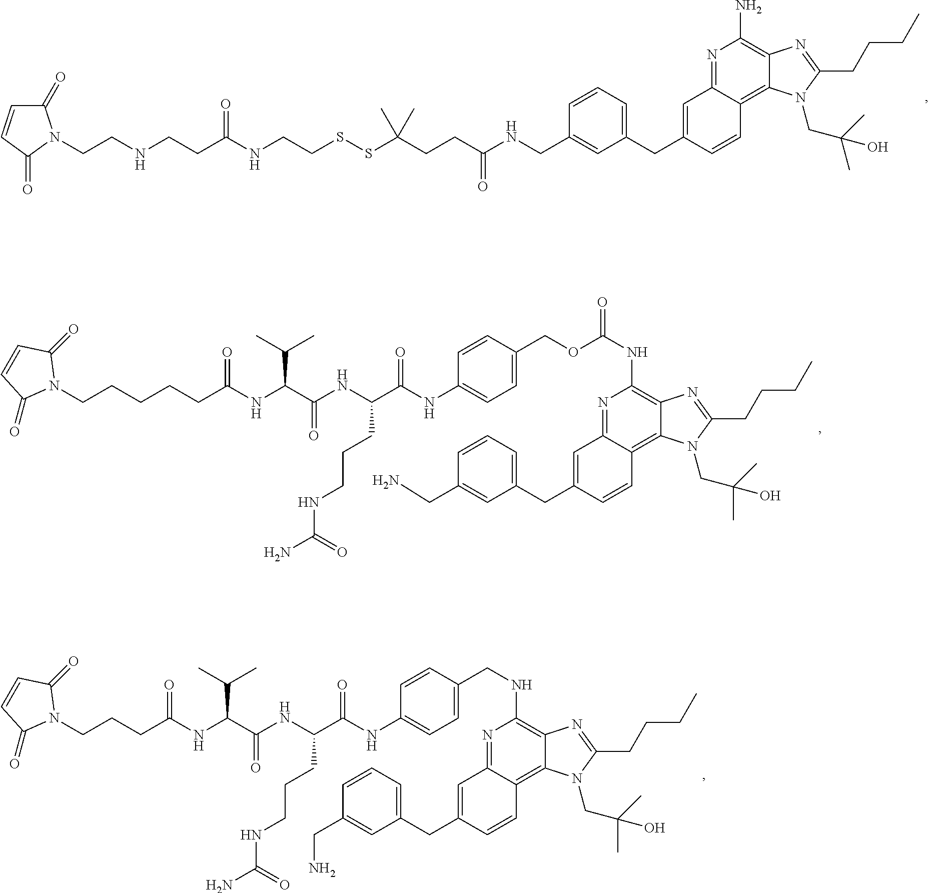 [SMILES: CCCCc1nc2c(N)nc3cc(Cc4cccc(CNC(=O)CCC(C)(C)SSCCNC(=O)CCNCCN5C(=O)C=CC5=O)c4)ccc3c2n1CC(C)(C)O ; CCCCc1nc2c(NC(=O)OCc3ccc(NC(=O)[C@H](CCCNC(N)=O)NC(=O)[C@@H](NC(=O)CCCCCN4C(=O)C=CC4=O)C(C)C)cc3)nc3cc(Cc4cccc(CN)c4)ccc3c2n1CC(C)(C)O ; CCCCc1nc2c(NCc3ccc(NC(=O)[C@H](CCCNC(N)=O)NC(=O)[C@@H](NC(=O)CCCN4C(=O)C=CC4=O)C(C)C)cc3)nc3cc(Cc4cccc(CN)c4)ccc3c2n1CC(C)(C)O]

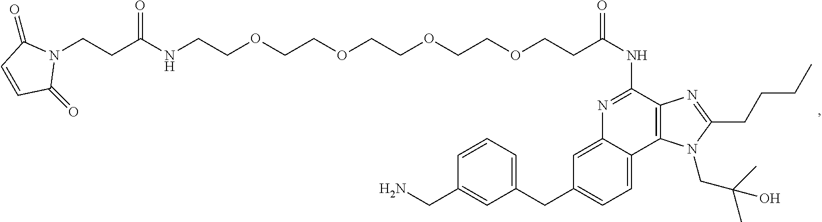 [SMILES: CCCCc1nc2c(NC(=O)CCOCCOCCOCCOCCNC(=O)CCN3C(=O)C=CC3=O)nc3cc(Cc4cccc(CN)c4)ccc3c2n1CC(C)(C)O]

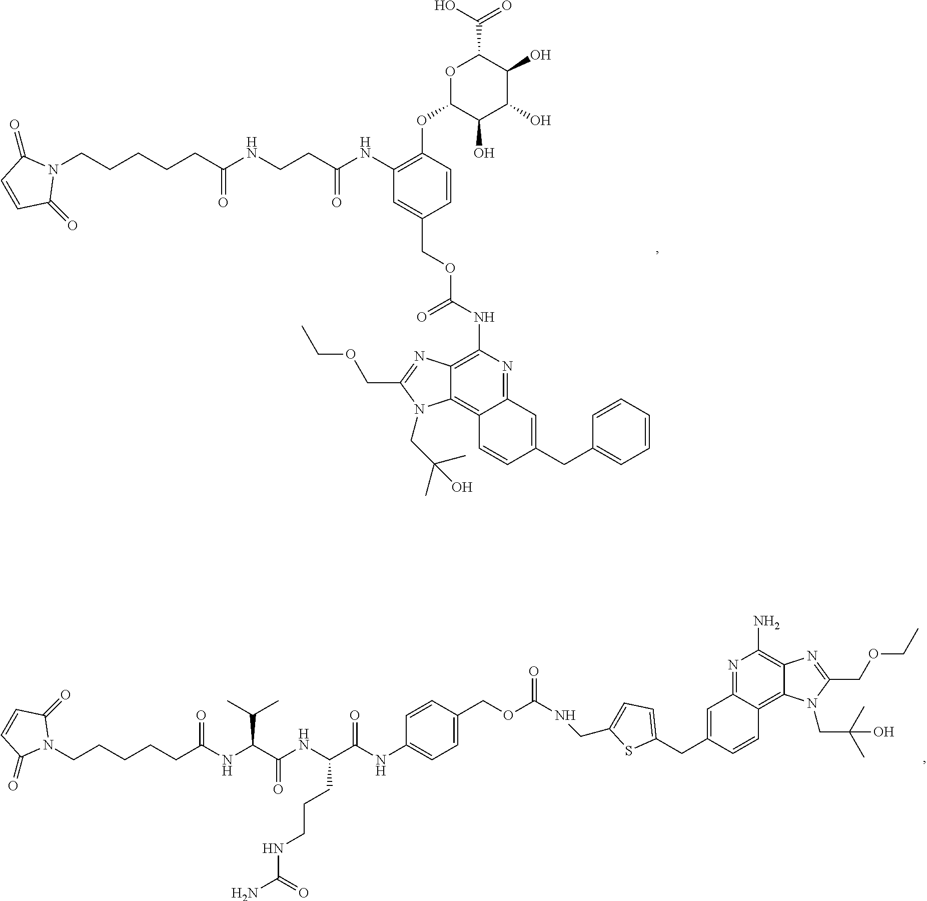 [SMILES: CCOCc1nc2c(NC(=O)OCc3ccc(O[C@@H]4O[C@H](C(=O)O)[C@@H](O)[C@H](O)[C@H]4O)c(NC(=O)CCNC(=O)CCCCCN4C(=O)C=CC4=O)c3)nc3cc(Cc4ccccc4)ccc3c2n1CC(C)(C)O ; CCOCc1nc2c(N)nc3cc(Cc4ccc(CNC(=O)OCc5ccc(NC(=O)[C@H](CCCNC(N)=O)NC(=O)[C@@H](NC(=O)CCCCCN6C(=O)C=CC6=O)C(C)C)cc5)s4)ccc3c2n1CC(C)(C)O]

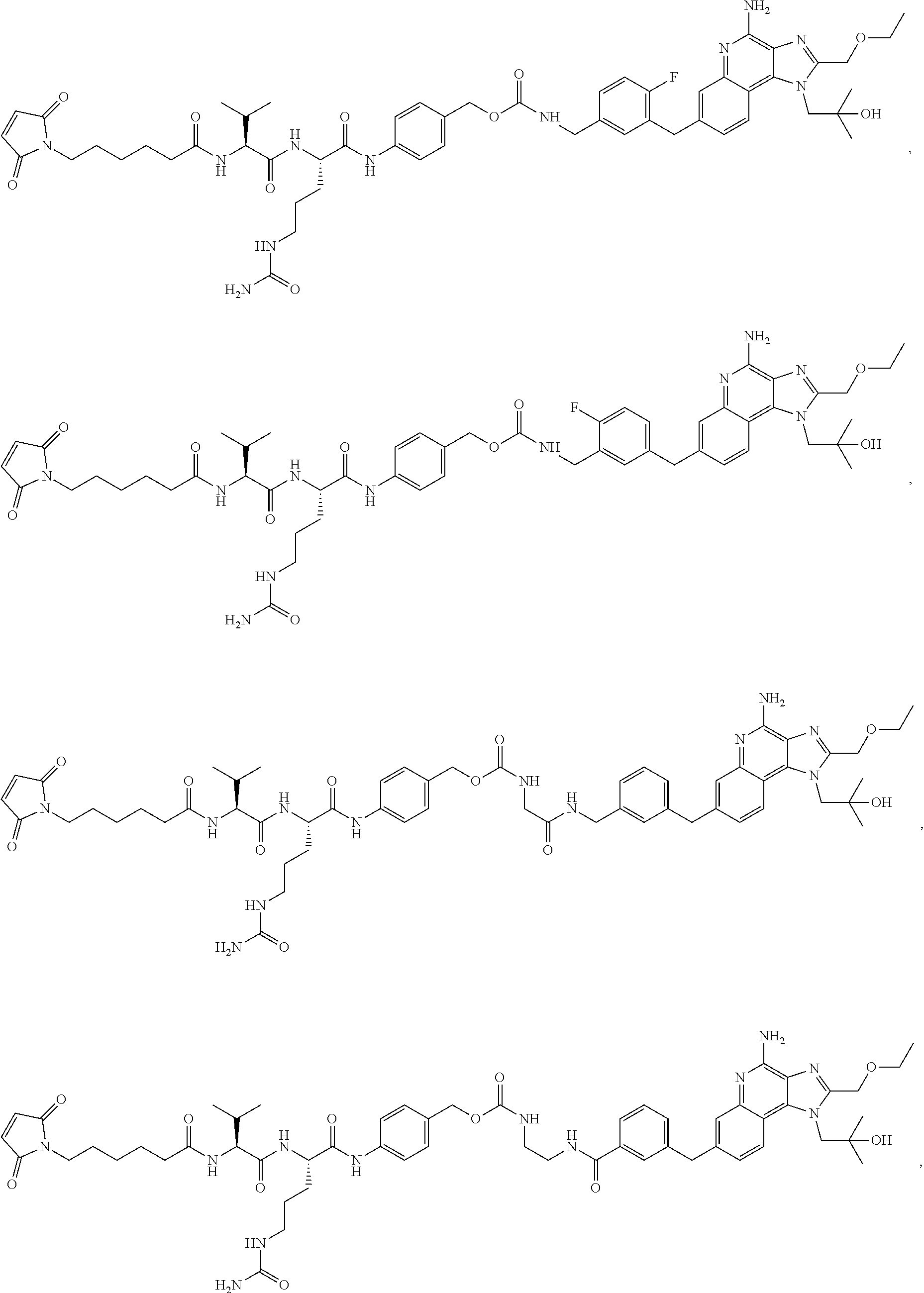

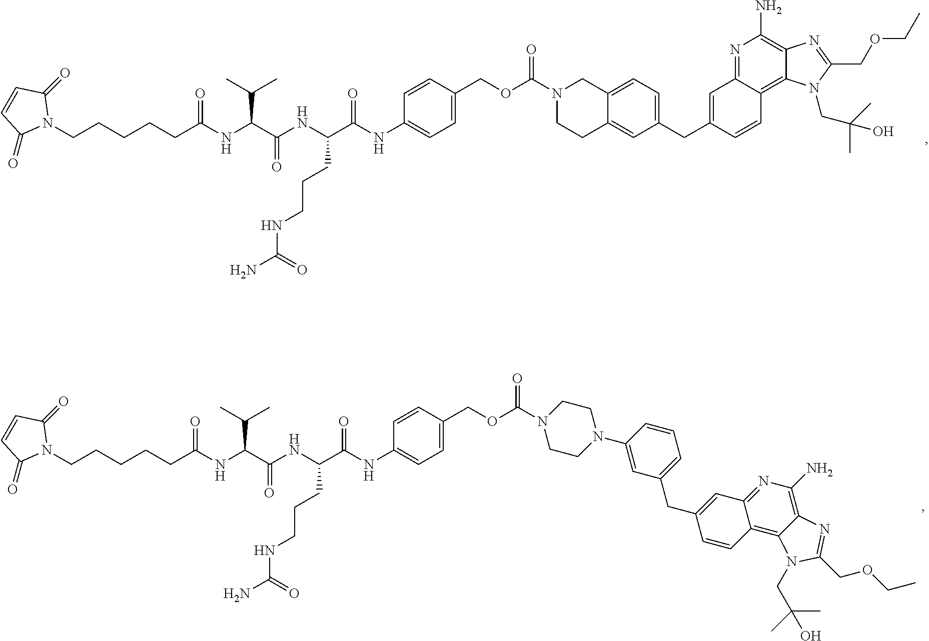 [SMILES: CCOCc1nc2c(N)nc3cc(Cc4ccc5c(c4)CCN(C(=O)OCc4ccc(NC(=O)[C@H](CCCNC(N)=O)NC(=O)[C@@H](NC(=O)CCCCCN6C(=O)C=CC6=O)C(C)C)cc4)C5)ccc3c2n1CC(C)(C)O ; CCOCc1nc2c(N)nc3cc(Cc4cccc(N5CCN(C(=O)OCc6ccc(NC(=O)[C@H](CCCNC(N)=O)NC(=O)[C@@H](NC(=O)CCCCCN7C(=O)C=CC7=O)C(C)C)cc6)CC5)c4)ccc3c2n1CC(C)(C)O]

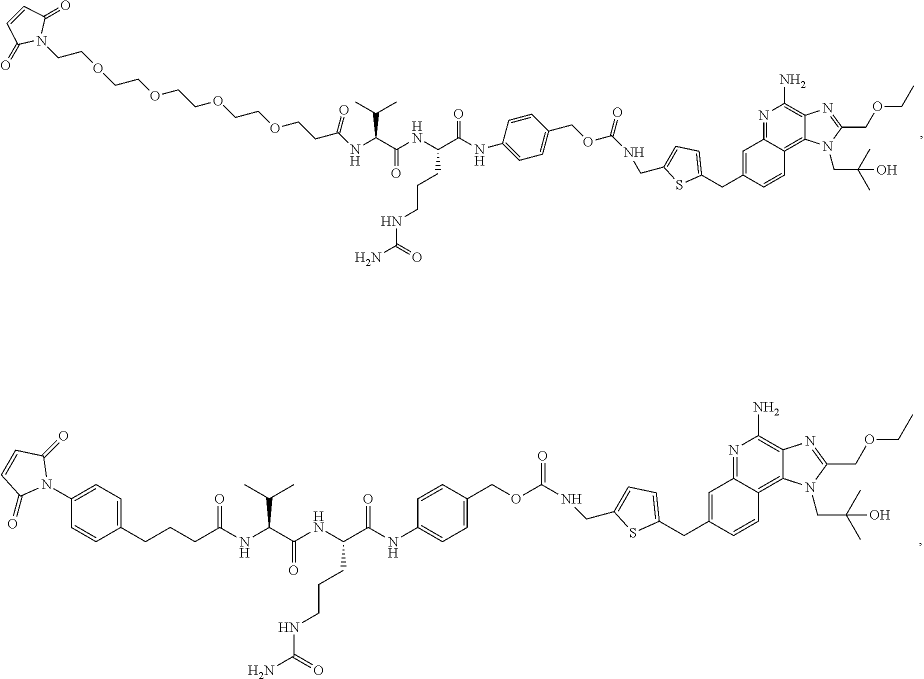 [SMILES: CCOCc1nc2c(N)nc3cc(Cc4ccc(CNC(=O)OCc5ccc(NC(=O)[C@H](CCCNC(N)=O)NC(=O)[C@@H](NC(=O)CCOCCOCCOCCOCCN6C(=O)C=CC6=O)C(C)C)cc5)s4)ccc3c2n1CC(C)(C)O ; CCOCc1nc2c(N)nc3cc(Cc4ccc(CNC(=O)OCc5ccc(NC(=O)[C@H](CCCNC(N)=O)NC(=O)[C@@H](NC(=O)CCCc6ccc(N7C(=O)C=CC7=O)cc6)C(C)C)cc5)s4)ccc3c2n1CC(C)(C)O]

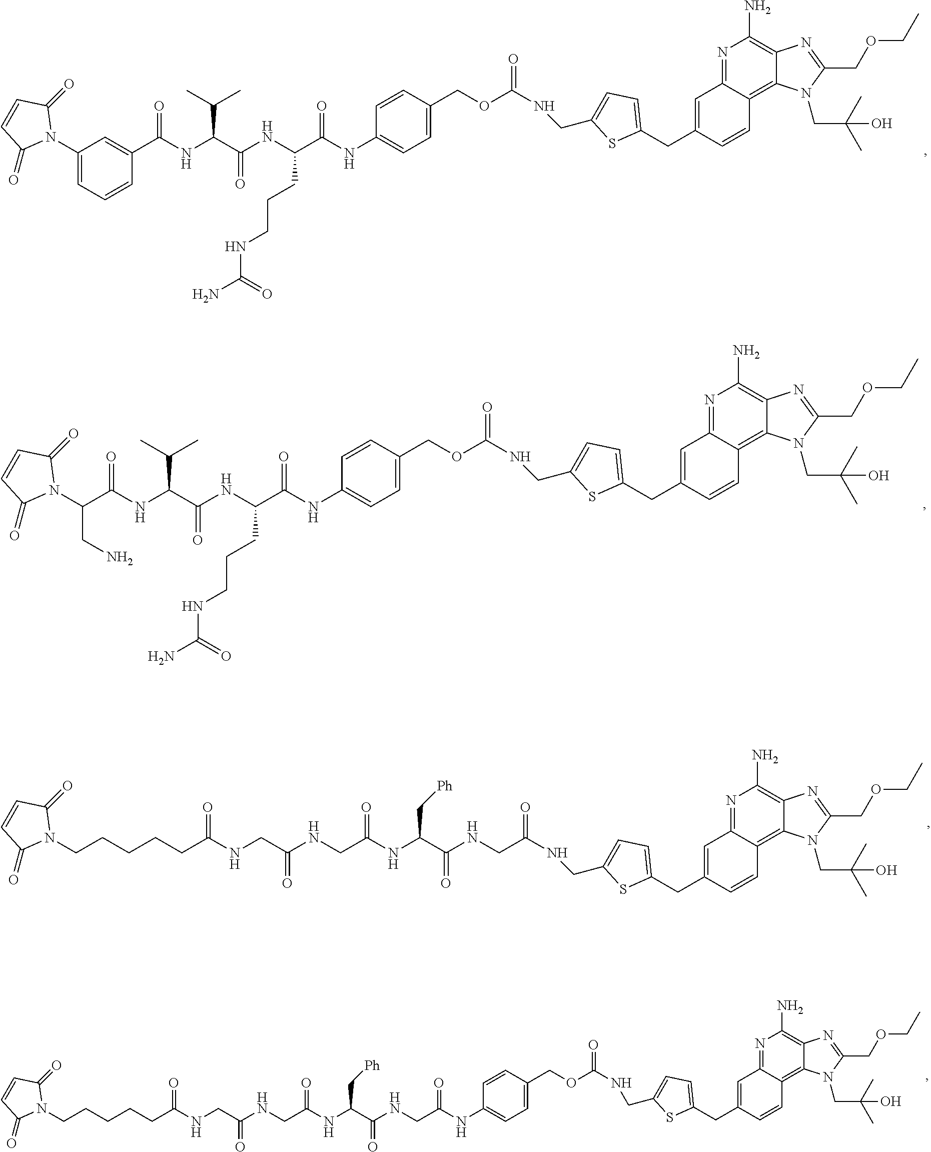

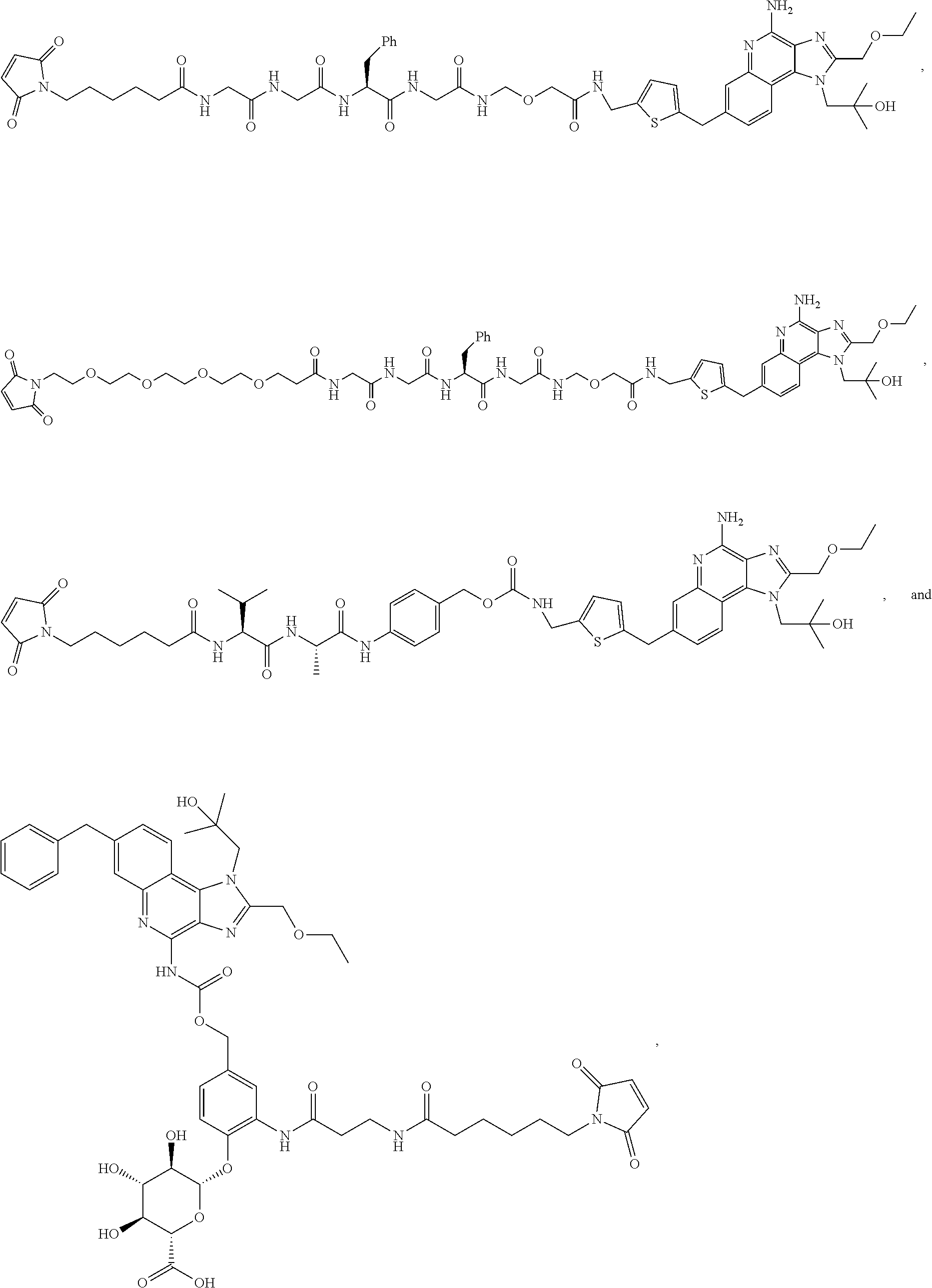

or a pharmaceutically acceptable salt thereof.

### Conjugate Compound

In another aspect, there is provided a conjugate compound having Formula (I):

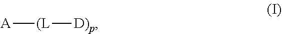

or a pharmaceutically acceptable salt thereof, wherein
A is a targeting moiety;
L is a linker;
p is an integer from 1 to 8;
D is a payload unit of Formula (II):

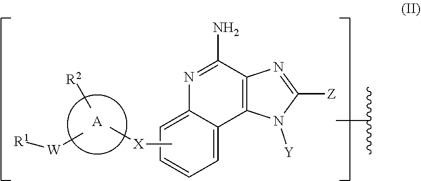

wherein:
   X is selected from the group consisting of —O—, —S—, —NH—, —(CH₂)ᵢ—, —(X¹)NC(O)—, —(X¹)NS(O)₂—, —C(O)N(X¹)— and —S(O)₂N(X¹)—, wherein —NH— and —(CH₂)ᵢ— are optionally substituted with one or more groups independently selected from halogen, hydroxyl, cyano, amino or alkyl;
   Ring A is cycloalkyl, heterocyclyl, aryl or heteroaryl;
   W is selected from the group consisting of a direct bond, alkyl, cycloalkyl, heterocyclyl, *—W¹-alkyl-, *-alkyl-W¹—, and *-alkyl-W¹-alkyl-, wherein the alkyl, cycloalkyl, heterocyclyl, and alkyl in *—W¹-alkyl-, *-alkyl-W¹—, and *-alkyl-W¹-alkyl are optionally substituted with one or more groups independently selected from halogen, hydroxyl, cyano, amino, alkyl and —C(O)OR^{a}, and wherein * end of W is connected to Ring A;
   W¹ is —O—, —NR^{a}—, —C(O)—, —C(O)NR^{a}—, —NR^{a}C(O)—, or —NR^{a}C(O)NR^{a}—,
      each R^{a} is independently hydrogen, alkyl, or haloalkyl;
   R¹ is hydrogen, —N(R^{b})₂, hydroxyl or SH;
      each R^{b} is independently hydrogen, alkyl, or haloalkyl, or
      two R^{b} taken together with the nitrogen atom to which they are bound form a heterocyclyl;
   R² is hydrogen, halogen, hydroxyl, cyano, amino, alkyl, alkenyl, alkynyl, alkoxyl, haloalkyl, —OR^{e}, —OC(O)R^{e}, —SR^{e}, —B(OH)₂, —NO₂, —CHO, —C(═NOR^{e})(R^{e}), —R²⁻¹—N(R^{e})₂, —R²⁻¹—N(R^{e})C(O)R^{e}, —R²⁻¹—N(R^{e})S(O)₂R^{e}, —R²⁻¹—N(R^{e})P(O)₂R^{e}, —R²⁻¹—C(O)OR^{e}, —R²⁻¹—C(O)N(R^{e})₂, —R²⁻¹—S(O)₂N(R^{e})₂, —R²⁻¹—P(O)₂N(R^{e})₂, —OC(O)NR^{e}, or —NC(O)NR^{e},
      each R²⁻¹ is independently null or alkyl;
      each R^{e} is independently hydrogen, alkyl, or haloalkyl;
   Y is —Y¹—Y²—Y³, wherein
      Y¹ is a direct bond or —(CH₂)ₘ-Q¹-(CH₂)ₙ—O—*, wherein —(CH₂)ₘ— and —(CH₂)ₙ— are optionally substituted with one or more groups independently selected from halogen, hydroxyl, alkyl or haloalkyl, and * end of Y¹ is connected to Y²;
      Y² is a direct bond or —(CH₂)ₛ-Q²-(CH₂)ₜ—NR^{c}—**, wherein —(CH₂)ₛ— and —(CH₂)ₜ— are optionally substituted with one or more groups independently selected from halogen, hydroxyl, alkyl or haloalkyl, and ** end of Y² is connect to Y³;
      Y³ is selected from the group consisting of hydrogen, alkyl, -alkyl-aryl, -alkyl-N(R^{e})₂, —C(O)-alkyl, —C(O)-alkyl-N(R^{b})₂, and —S(O)₂-alkyl;
      Q¹ and Q² are each independently selected from a direct bond, cycloalkyl, heterocyclyl, aryl, or heteroaryl, wherein the cycloalkyl, heterocyclyl, aryl, and heteroaryl are optionally substituted with one or more groups independently selected from halogen, hydroxyl, cyano, amino, or alkyl;
      R^{c} is hydrogen or alkyl, or
      R^{c} and Y³ taken together with the atoms to which they are bound form a heterocyclyl optionally substituted with one or more groups independently selected from halogen, hydroxyl, cyano, amino or alkyl;
   Z is selected from the group consisting of alkyl, alkenyl, alkynyl, heteroalkyl, heteroalkenyl and heteroalkynyl, each of which is optionally substituted with one or more R^{d};
      R^{d} is selected from the group consisting of halogen, acyl, alkyl, alkenyl, alkynyl, cycloalkyl and —O-cycloalkyl;
   i is 0, 1, 2, 3, 4, 5 or 6;
   m is 0, 1,2,3,4 or 5;
   n is 0, 1, 2, 3, 4 or 5;
   s is 0, 1, 2, 3, 4 or 5; and
   t is 0, 1, 2, 3, 4 or 5.

In some embodiments, ring A is selected from the group consisting of:

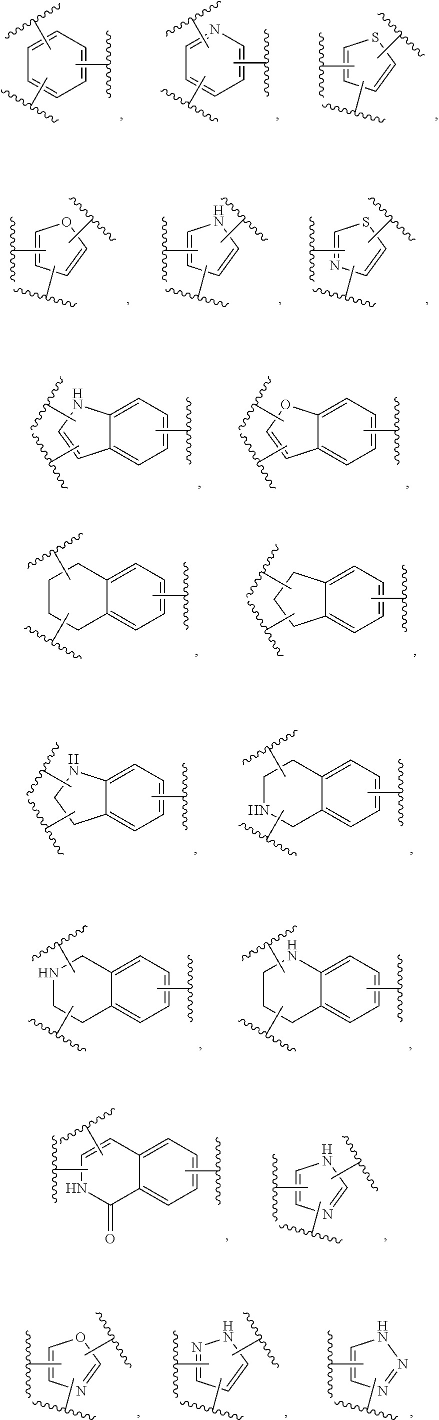

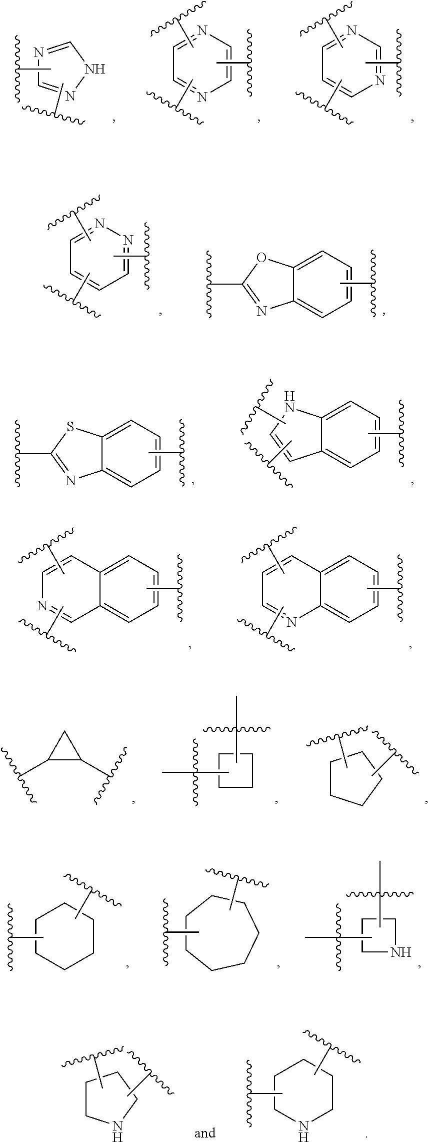

In some embodiments, X is —O—, —NH—, or —(CH₂)ᵢ—, wherein —NH— and —(CH₂)ᵢ— are optionally substituted with one or more halogen or alkyl.

In certain embodiments, X is —O—, —N(CH₃)—, —CH₂—, (CH₂)₂—, (CH₂)₃—, (CH₂)₄—, —C(CH₃)₂— or —CF₂—.

In some embodiments, R¹ is hydrogen, —N(R^{b})₂, or hydroxyl.

In some embodiments, W is a direct bond.

In certain embodiments, W is a direct bond, R¹ is hydrogen, —N(R^{b})₂ or hydroxyl, and each R^{b} is independently hydrogen or alkyl, or two R^{b} taken together with the nitrogen atom to which they are bound form a heterocyclyl.

In some embodiments, W is alkyl optionally substituted with —C(O)OR^{a}. In certain embodiments, W is C₁₋₆ alkyl, C₁₋₅ alkyl, C₁₋₄ alkyl, C₁₋₃ alkyl, or C₁₋₂ alkyl, optionally substituted with —C(O)OR^{a}. In certain embodiments, W is methyl, ethyl or propyl, each optionally substituted with —C(O)OR^{a}. In certain embodiments, R^{a} is C₁₋₆ alkyl, C₁₋₅ alkyl, C₁₋₄ alkyl, C₁₋₃ alkyl, or C₁₋₂ alkyl.

In certain embodiments, W is alkyl optionally substituted with —C(O)OR^{a}, R¹ is hydrogen, hydroxyl or —N(R^{b})₂, and each R^{b} is independently hydrogen or alkyl, or two R^{b} taken together with the nitrogen atom to which they are bound form a heterocyclyl. In certain embodiments, R¹ is hydrogen. In certain embodiments, R¹ is hydroxyl. In certain embodiments, R¹ is —N(R^{b})₂, and each R^{b} is independently hydrogen, C₁₋₆ alkyl, C₁₋₅ alkyl, C₁₋₄ alkyl, C₁₋₃ alkyl, or C₁₋₂ alkyl. In certain embodiments, R¹ is —N(R^{b})₂, and each R^{b} is independently hydrogen or methyl. In certain embodiments, R¹ is —N(R^{b})₂, and two R^{b} taken together with the nitrogen atom to which they are bound form a 3 to 10 membered heterocyclyl, 3 to 9 membered heterocyclyl, 3 to 8 membered heterocyclyl, 3 to 7 membered heterocyclyl, 3 to 6 membered heterocyclyl, or 3 to 5 membered heterocyclyl.

In some embodiments, W is *—W¹-alkyl-. In certain embodiments, W is *—W¹—C₁₋₆ alkyl-, *—W¹—C₁₋₅ alkyl-, *—W¹—C₁₋₄ alkyl-, *—W¹—C₁₋₃ alkyl-, or *—W¹—C₁₋₂ alkyl-.

In certain embodiments, W is *—W¹-alkyl-, W¹ is —O—, —NR^{a}—, —C(O)NR^{a}—, —OC(O)NR^{a}—, —NR^{a}C(O)—, —NR^{a}C(O)O—, or —NR^{a}C(O)NR^{a}—, and each R^{a} is independently hydrogen, alkyl, or haloalkyl.

In certain embodiments, W is *—W¹-alkyl-, and R¹ is —NH₂ or —NH—CH₃.

In some embodiments, W is *-alkyl-W¹—. In certain embodiments, W is *—C₁₋₆ alkyl-W¹—, *—C₁₋₅ alkyl-W¹—, *—C₁₋₄ alkyl-W¹—, *—C₁₋₃ alkyl-W¹—, or *—C₁₋₂ alkyl-W¹—.

In certain embodiments, W is *-alkyl-W¹—, and W¹ is —C(O)—.

In certain embodiments, W is *-alkyl-W¹—, W¹ is —C(O)—, and R¹ is —NH₂.

In some embodiments, W is *-alkyl-W¹-alkyl-. In certain embodiments, W is *-alkyl-W¹-alkyl-, and each alkyl in W is independently C₁₋₆ alkyl, C₁₋₆ alkyl, C₁₋₄ alkyl, C₁₋₃ alkyl, or C₁₋₂ alkyl.

In certain embodiments, W is *-alkyl-W¹-alkyl-, W¹ is —NR^{a}C(O)— or —OC(O)NR^{a}—, and R^{a} is hydrogen, alkyl, or haloalkyl.

In certain embodiments, W is *-alkyl-W¹-alkyl-, W¹ is —NR^{a}C(O)—, and R¹ is —NH₂.

In certain embodiments, W is *-alkyl-W¹-alkyl-, W¹ is —NR^{a}C(O)—, and R¹ is hydroxyl.

In embodiments, W is *-alkyl-W¹-alkyl-, W¹ is —OC(O)NR^{a}—, and R¹ is —NH—CH₃.

In some embodiments, W is cycloalkyl. In certain embodiments, W is C₃₋₁₀ cycloalkyl, C₃₋₉ cycloalkyl, C₃₋₅ cycloalkyl, C₃₋₇ cycloalkyl, C₃₋₆ cycloalkyl, or C₃₋₅ cycloalkyl.

In certain embodiments, W is cycloalkyl, and R¹ is —NH₂.

In some embodiment, W is heterocyclyl. In certain embodiments, W is 3 to 10 membered heterocyclyl, 3 to 9 membered heterocyclyl, 3 to 8 membered heterocyclyl, 3 to 7 membered heterocyclyl, 3 to 6 membered heterocyclyl, or 3 to 5 membered heterocyclyl.

In certain embodiments, W is heterocyclyl, and R¹ is hydrogen.

In some embodiments, R² is hydrogen, halogen, cyano, alkyl or alkoxyl. In certain embodiments, R² is hydrogen.

In some embodiments, Y¹ is a direct bond.

In certain embodiments, Y¹ is a direct bond, and Y² is a direct bond.

In certain embodiments, Y¹ is a direct bond, Y² is a direct bond, and Y³ is -alkyl-aryl.

In certain embodiments, Y¹ is a direct bond, and Y² is —(CH₂)ₛ-Q²-(CH₂)ₜ—NR^{c}—**. In certain embodiments, Q² is a direct bond, cycloalkyl or aryl, wherein the cycloalkyl or aryl is optionally substituted with one or more groups independently selected from halogen or alkyl. In certain embodiments, R¹ is hydrogen.

In certain embodiments, Y¹ is a direct bond, Y² is —(CH₂)ₛ-Q²-(CH₂)ₜ—NR^{c}—**, and Y³ is hydrogen, -alkyl-NH₂, or —S(O)₂-alkyl. In certain embodiments, the alkyl in -alkyl-NH₂ and —S(O)₂-alkyl is independently C₁₋₆ alkyl, C₁₋₅ alkyl, C₁₋₄ alkyl, C₁₋₃ alkyl, or C₁₋₂ alkyl.

In some embodiments, Y¹ is —(CH₂)ₘ-Q¹-(CH₂)ₙ—O—*, wherein —(CH₂)ₘ— and —(CH₂)ₙ— are optionally substituted with one or more groups independently selected from halogen, hydroxyl, alkyl or haloalkyl. In certain embodiments, —(CH₂)ₘ— and —(CH₂)ₙ— are optionally substituted with one or more groups independently selected from alkyl or haloalkyl. In certain embodiments, —(CH₂)ₘ— and —(CH₂)ₙ— are optionally substituted with one or more C₁₋₆ alkyl or C₁₋₆ haloalkyl. In certain embodiments, —(CH₂)ₘ— and —(CH₂)ₙ— are optionally substituted with one or more groups independently selected from methyl, ethyl or trifluoromethyl. In certain embodiments, Q¹ is a direct bond, cycloalkyl or aryl, wherein the cycloalkyl or aryl is optionally substituted with one or more groups independently selected from halogen or alkyl.

In certain embodiments, Y¹ is —(CH₂)ₘ-Q¹-(CH₂)ₙ—O—*, wherein —(CH₂)ₘ— and —(CH₂)ₙ— are optionally substituted with one or more groups independently selected from halogen, hydroxyl, alkyl or haloalkyl, and Y² is a direct bond. In certain embodiments, —(CH₂)ₘ— and —(CH₂)ₙ— are optionally substituted with one or more groups independently selected from alkyl or haloalkyl. In certain embodiments, —(CH₂)ₘ— and —(CH₂)ₙ— are optionally substituted with one or more C₁₋₆ alkyl or C₁₋₆ haloalkyl. In certain embodiments, —(CH₂)ₘ— and —(CH₂)ₙ— are optionally substituted with one or more groups independently selected from methyl, ethyl or trifluoromethyl.

In certain embodiments, Y¹ is —(CH₂)ₘ-Q¹-(CH₂)ₙ—O—*, Y² is a direct bond, and Y³ is hydrogen. In certain embodiments, —(CH₂)ₘ— and —(CH₂)ₙ— are optionally substituted with one or more groups independently selected from alkyl or haloalkyl. In certain embodiments, —(CH₂)ₘ— and —(CH₂)ₙ— are optionally substituted with one or more C₁₋₆ alkyl or C₁₋₆ haloalkyl. In certain embodiments, —(CH₂)ₘ— and —(CH₂)ₙ— are optionally substituted with one or more groups independently selected from methyl, ethyl or trifluoromethyl.

In certain embodiments, Y¹ is —(CH₂)ₘ-Q¹-(CH₂)ₙ—O—*, and Y² is —(CH₂)ₛ-Q²-(CH₂)ₜ—NR^{c}—**. In certain embodiments, Q² is a direct bond. In certain embodiments, R^{c} is hydrogen. In certain embodiments, —(CH₂)ₘ— and —(CH₂)ₙ— are optionally substituted with one or more groups independently selected from alkyl or haloalkyl. In certain embodiments, —(CH₂)ₘ— and —(CH₂)ₙ— are optionally substituted with one or more C₁₋₆ alkyl or C₁₋₆ haloalkyl. In certain embodiments, —(CH₂)ₘ— and —(CH₂)ₙ— are optionally substituted with one or more groups independently selected from methyl, ethyl or trifluoromethyl.

In certain embodiments, Y¹ is —(CH₂)ₘ-Q¹-(CH₂)ₙ—O—*, Y² is —(CH₂)ₛ-Q²-(CH₂)ₜ—NR^{c}—**, and Y³ is hydrogen, —C(O)-alkyl or —C(O)-alkyl-N(R^{b})₂. In certain embodiments, —(CH₂)ₘ— and —(CH₂)ₙ— are optionally substituted with one or more groups independently selected from alkyl or haloalkyl. In certain embodiments, —(CH₂)ₘ— and —(CH₂)ₙ— are optionally substituted with one or more C₁₋₆ alkyl or C₁₋₆ haloalkyl. In certain embodiments, —(CH₂)ₘ— and —(CH₂)ₙ— are optionally substituted with one or more groups independently selected from methyl, ethyl or trifluoromethyl. In certain embodiments, R^{c} and Y³ taken together with the atoms to which they are bound form a heterocyclyl optionally substituted with one or more halogen.

In some embodiments, Z is selected from the group consisting of alkyl, alkenyl, alkynyl and heteroalkyl, which is optionally substituted with one or more R^{d}. In certain embodiments, Z is selected from the group consisting of C₁₋₆ alkyl, C₁₋₆ alkenyl, C₁₋₆ alkynyl and C₁₋₆ heteroalkyl, which is optionally substituted with one or more R^{d}.

In certain embodiments, R^{d} is selected from the group consisting of halogen, acyl, alkyl, cycloalkyl and —O-cycloalkyl. In certain embodiments, R^{d} is selected from the group consisting of halogen, acyl, C₁₋₆ alkyl, C₃₋₆ cycloalkyl and —O—C₃₋₆ cycloalkyl.

In some embodiments, D is a payload unit of Formula (IIa):

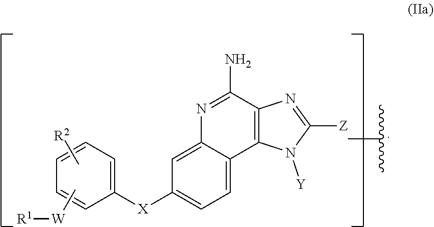

In some embodiments, the payload unit is selected from the group consisting of:

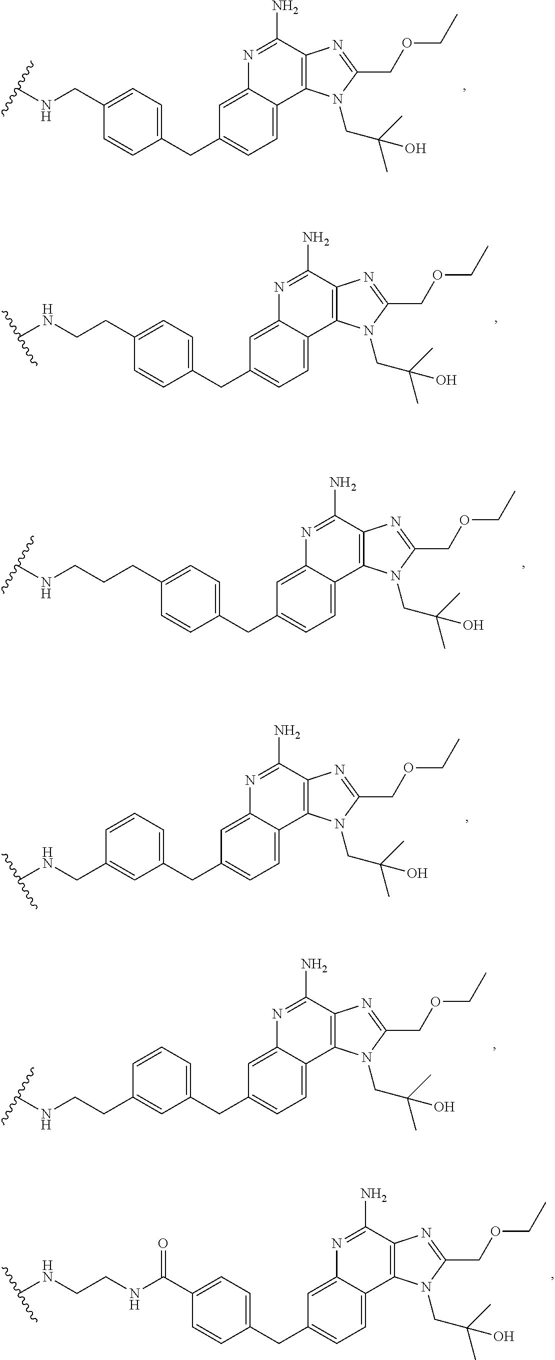 [SMILES: *NCc1ccc(Cc2ccc3c(c2)nc(N)c2nc(COCC)n(CC(C)(C)O)c23)cc1 ; *NCCc1ccc(Cc2ccc3c(c2)nc(N)c2nc(COCC)n(CC(C)(C)O)c23)cc1 ; *NCCCc1ccc(Cc2ccc3c(c2)nc(N)c2nc(COCC)n(CC(C)(C)O)c23)cc1 ; *NCc1cccc(Cc2ccc3c(c2)nc(N)c2nc(COCC)n(CC(C)(C)O)c23)c1 ; *NCCc1cccc(Cc2ccc3c(c2)nc(N)c2nc(COCC)n(CC(C)(C)O)c23)c1 ; *NCCNC(=O)c1ccc(Cc2ccc3c(c2)nc(N)c2nc(COCC)n(CC(C)(C)O)c23)cc1]

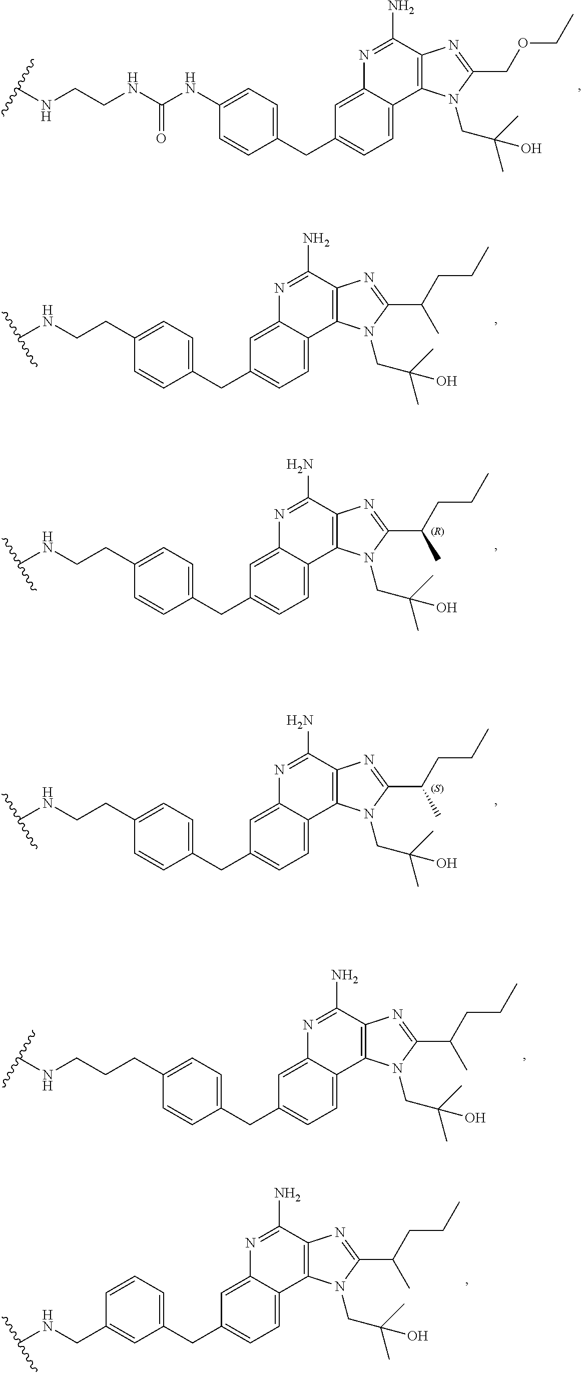

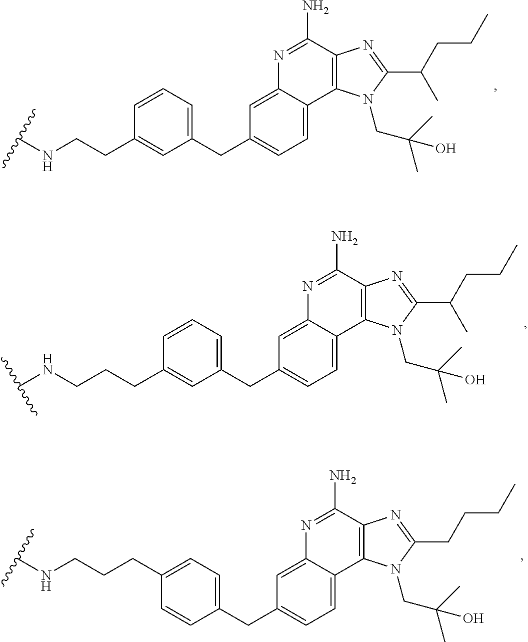 [SMILES: *NCCc1cccc(Cc2ccc3c(c2)nc(N)c2nc(C(C)CCC)n(CC(C)(C)O)c23)c1 ; *NCCCc1cccc(Cc2ccc3c(c2)nc(N)c2nc(C(C)CCC)n(CC(C)(C)O)c23)c1 ; *NCCCc1ccc(Cc2ccc3c(c2)nc(N)c2nc(CCCC)n(CC(C)(C)O)c23)cc1]

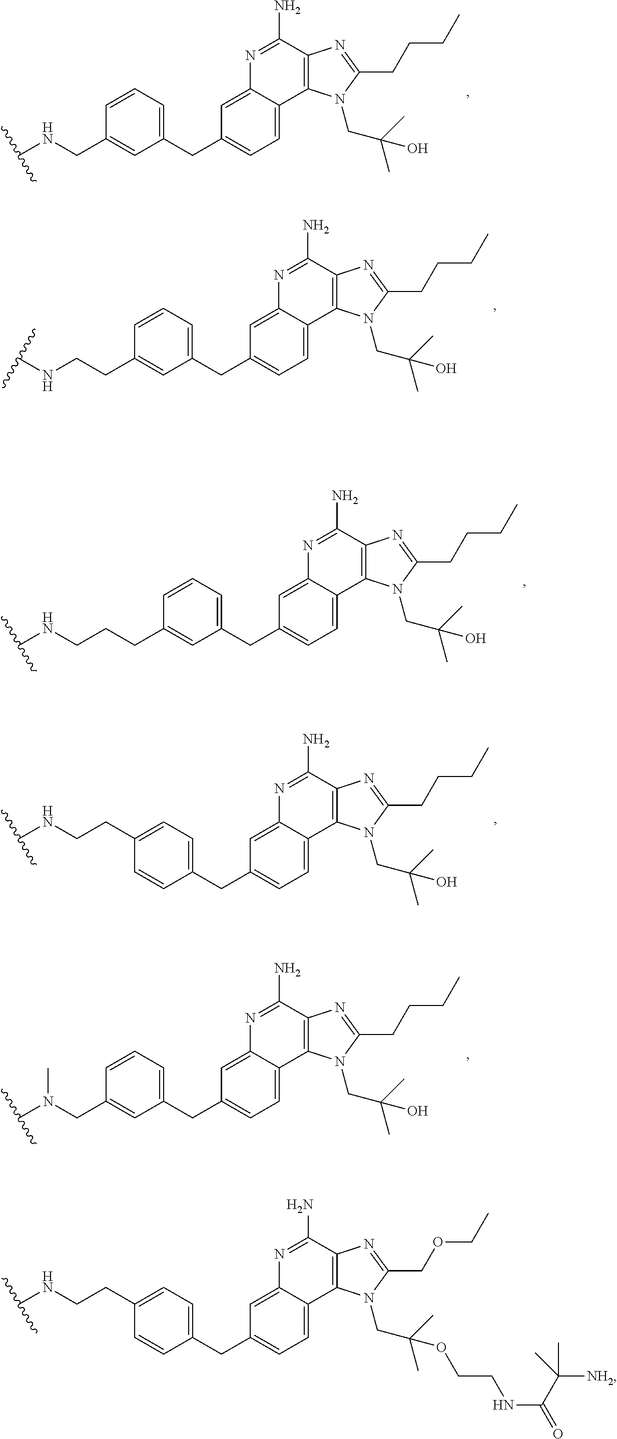 [SMILES: *NCc1cccc(Cc2ccc3c(c2)nc(N)c2nc(CCCC)n(CC(C)(C)O)c23)c1 ; *NCCc1cccc(Cc2ccc3c(c2)nc(N)c2nc(CCCC)n(CC(C)(C)O)c23)c1 ; *NCCCc1cccc(Cc2ccc3c(c2)nc(N)c2nc(CCCC)n(CC(C)(C)O)c23)c1 ; *NCCc1ccc(Cc2ccc3c(c2)nc(N)c2nc(CCCC)n(CC(C)(C)O)c23)cc1 ; *N(C)Cc1cccc(Cc2ccc3c(c2)nc(N)c2nc(CCCC)n(CC(C)(C)O)c23)c1 ; *NCCc1ccc(Cc2ccc3c(c2)nc(N)c2nc(COCC)n(CC(C)(C)OCCNC(=O)C(C)(C)N)c23)cc1]

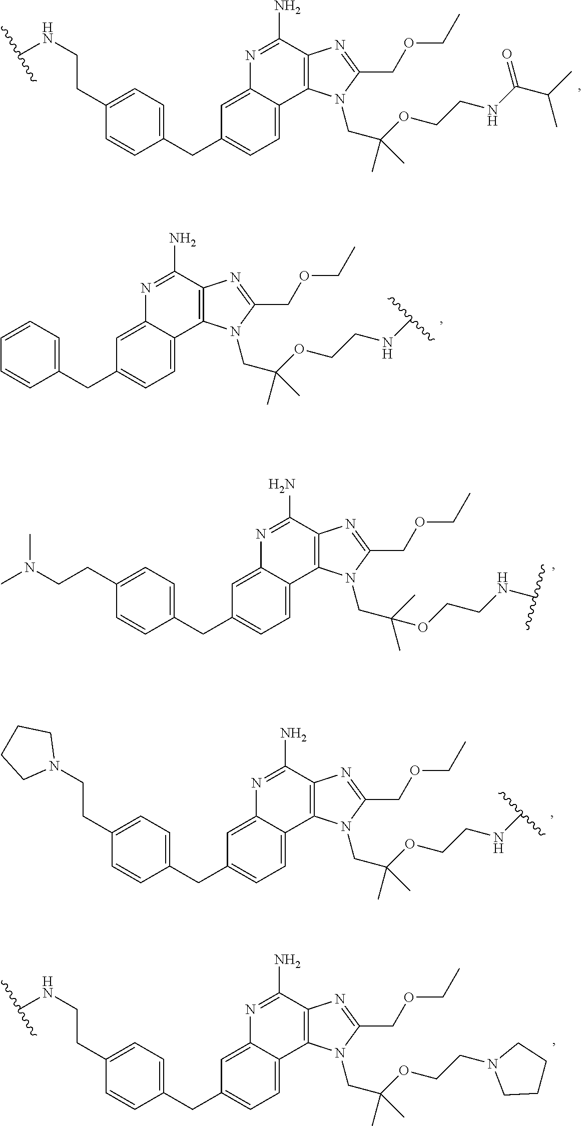

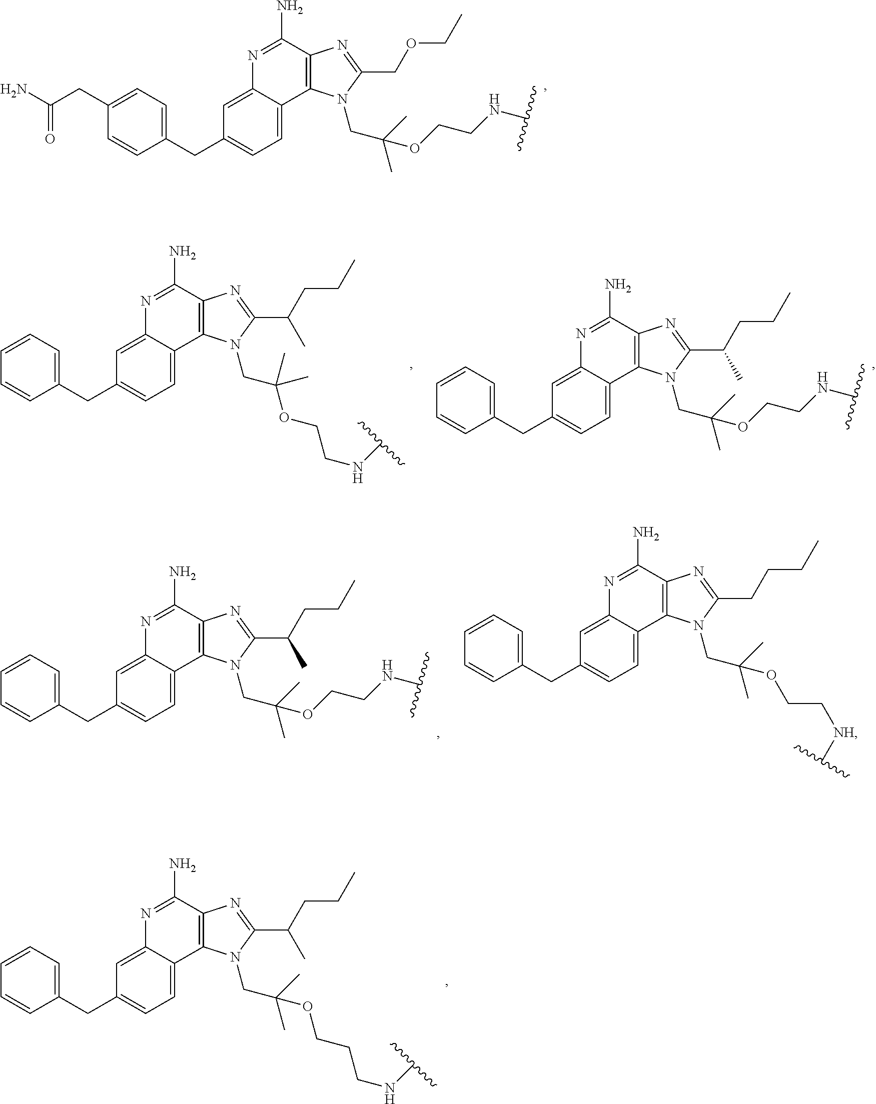 [SMILES: *NCCOC(C)(C)Cn1c(COCC)nc2c(N)nc3cc(Cc4ccc(CC(N)=O)cc4)ccc3c21 ; *NCCOC(C)(C)Cn1c(C(C)CCC)nc2c(N)nc3cc(Cc4ccccc4)ccc3c21 ; *NCCOC(C)(C)Cn1c([C@@H](C)CCC)nc2c(N)nc3cc(Cc4ccccc4)ccc3c21 ; *NCCOC(C)(C)Cn1c([C@H](C)CCC)nc2c(N)nc3cc(Cc4ccccc4)ccc3c21 ; *NCCOC(C)(C)Cn1c(CCCC)nc2c(N)nc3cc(Cc4ccccc4)ccc3c21 ; *NCCCOC(C)(C)Cn1c(C(C)CCC)nc2c(N)nc3cc(Cc4ccccc4)ccc3c21]

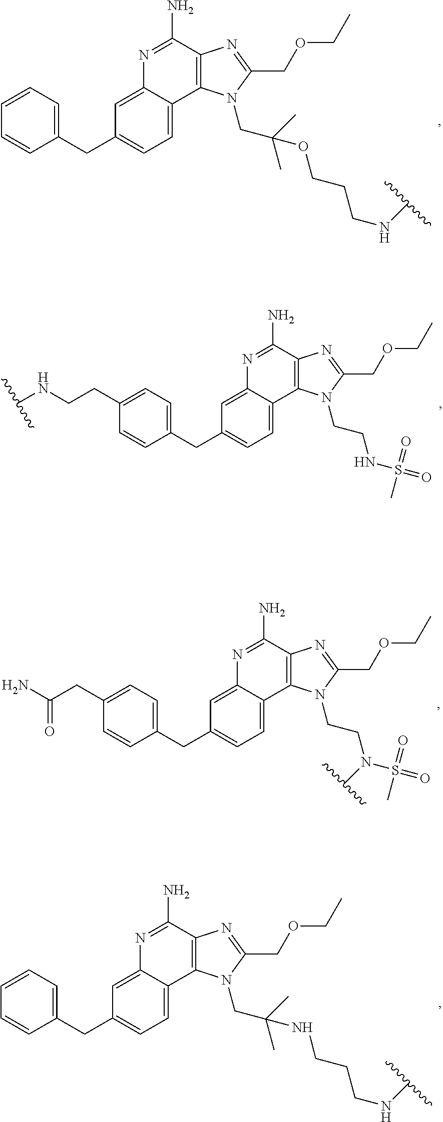 [SMILES: *NCCCOC(C)(C)Cn1c(COCC)nc2c(N)nc3cc(Cc4ccccc4)ccc3c21 ; *NCCc1ccc(Cc2ccc3c(c2)nc(N)c2nc(COCC)n(CCNS(C)(=O)=O)c23)cc1 ; *N(CCn1c(COCC)nc2c(N)nc3cc(Cc4ccc(CC(N)=O)cc4)ccc3c21)S(C)(=O)=O ; *NCCCNC(C)(C)Cn1c(COCC)nc2c(N)nc3cc(Cc4ccccc4)ccc3c21]

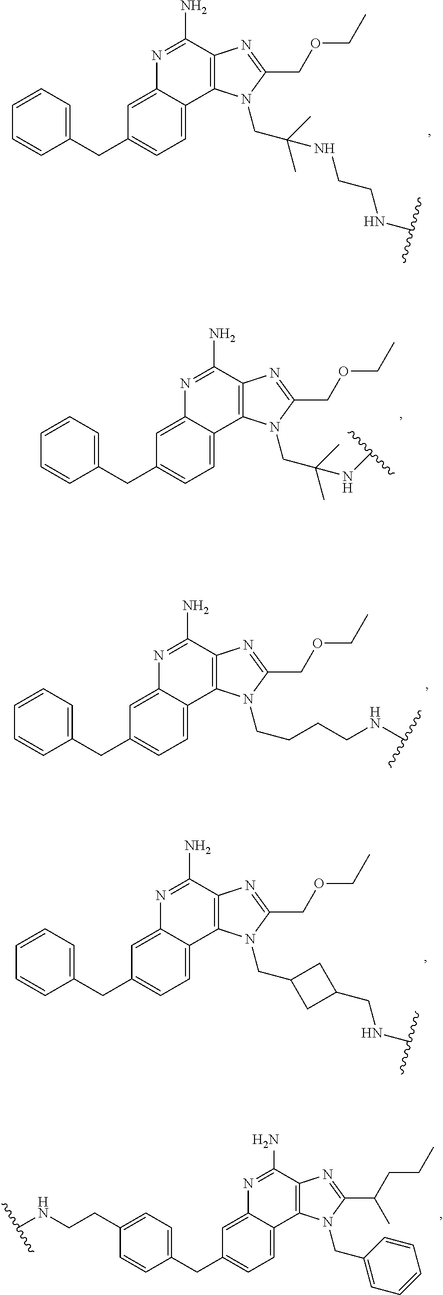 [SMILES: *NCCNC(C)(C)Cn1c(COCC)nc2c(N)nc3cc(Cc4ccccc4)ccc3c21 ; *NC(C)(C)Cn1c(COCC)nc2c(N)nc3cc(Cc4ccccc4)ccc3c21 ; *NCCCCn1c(COCC)nc2c(N)nc3cc(Cc4ccccc4)ccc3c21 ; *NCC1CC(Cn2c(COCC)nc3c(N)nc4cc(Cc5ccccc5)ccc4c32)C1 ; *NCCc1ccc(Cc2ccc3c(c2)nc(N)c2nc(C(C)CCC)n(Cc4ccccc4)c23)cc1]

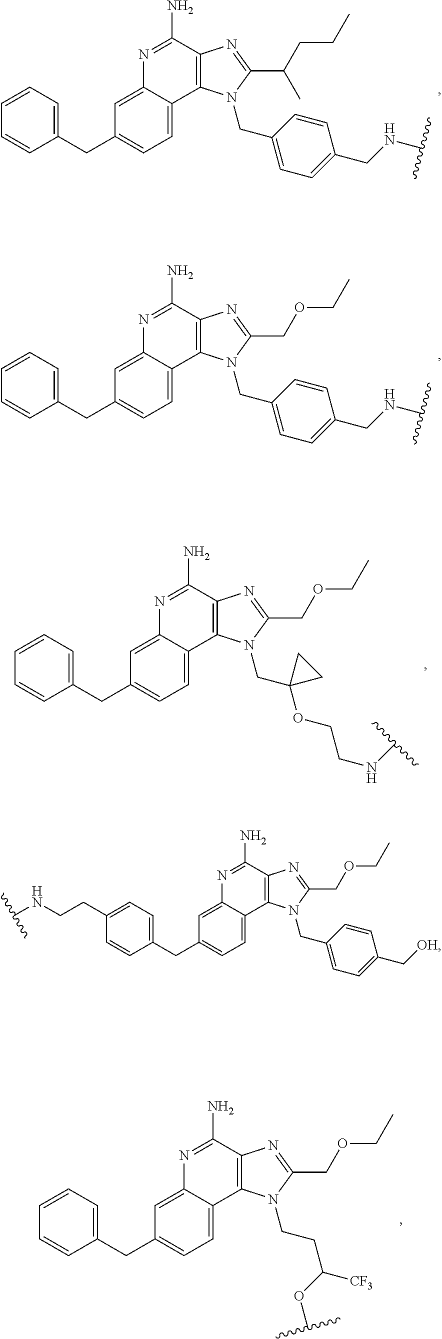 [SMILES: *NCc1ccc(Cn2c(C(C)CCC)nc3c(N)nc4cc(Cc5ccccc5)ccc4c32)cc1 ; *NCc1ccc(Cn2c(COCC)nc3c(N)nc4cc(Cc5ccccc5)ccc4c32)cc1 ; *NCCOC1(Cn2c(COCC)nc3c(N)nc4cc(Cc5ccccc5)ccc4c32)CC1 ; *NCCc1ccc(Cc2ccc3c(c2)nc(N)c2nc(COCC)n(Cc4ccc(CO)cc4)c23)cc1 ; *OC(CCn1c(COCC)nc2c(N)nc3cc(Cc4ccccc4)ccc3c21)C(F)(F)F]

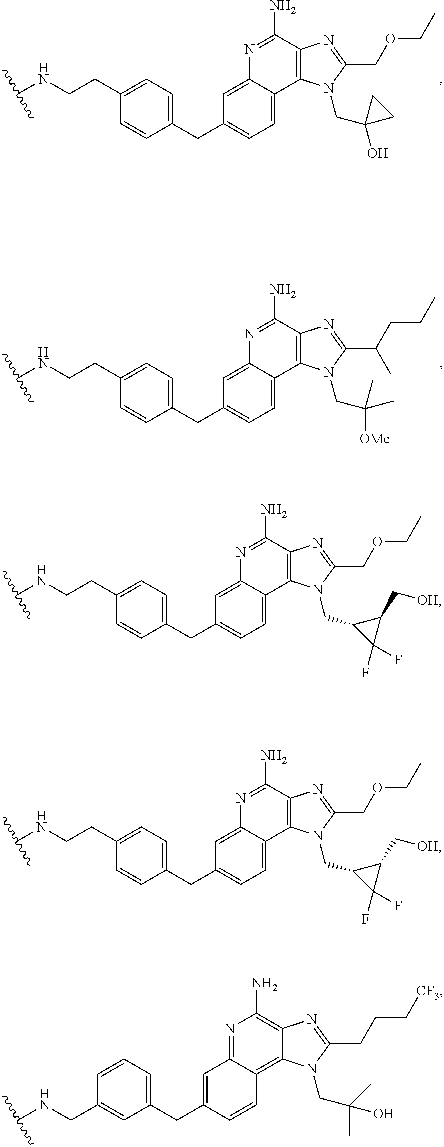 [SMILES: *NCCc1ccc(Cc2ccc3c(c2)nc(N)c2nc(COCC)n(CC4(O)CC4)c23)cc1 ; *NCCc1ccc(Cc2ccc3c(c2)nc(N)c2nc(C(C)CCC)n(CC(C)(C)OC)c23)cc1 ; *NCCc1ccc(Cc2ccc3c(c2)nc(N)c2nc(COCC)n(C[C@@H]4[C@@H](CO)C4(F)F)c23)cc1 ; *NCCc1ccc(Cc2ccc3c(c2)nc(N)c2nc(COCC)n(C[C@@H]4[C@H](CO)C4(F)F)c23)cc1 ; *NCc1cccc(Cc2ccc3c(c2)nc(N)c2nc(CCCC(F)(F)F)n(CC(C)(C)O)c23)c1]

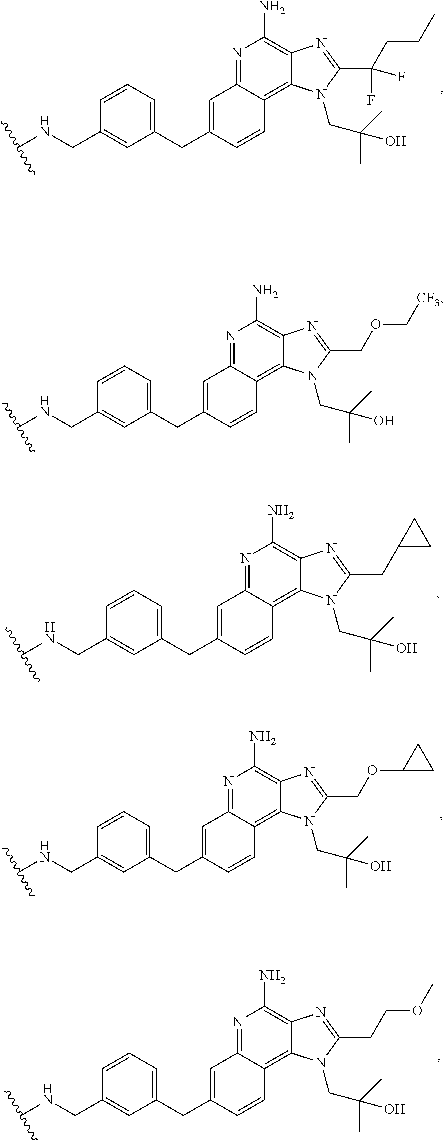 [SMILES: *NCc1cccc(Cc2ccc3c(c2)nc(N)c2nc(C(F)(F)CCC)n(CC(C)(C)O)c23)c1 ; *NCc1cccc(Cc2ccc3c(c2)nc(N)c2nc(COCC(F)(F)F)n(CC(C)(C)O)c23)c1 ; *NCc1cccc(Cc2ccc3c(c2)nc(N)c2nc(CC4CC4)n(CC(C)(C)O)c23)c1 ; *NCc1cccc(Cc2ccc3c(c2)nc(N)c2nc(COC4CC4)n(CC(C)(C)O)c23)c1 ; *NCc1cccc(Cc2ccc3c(c2)nc(N)c2nc(CCOC)n(CC(C)(C)O)c23)c1]

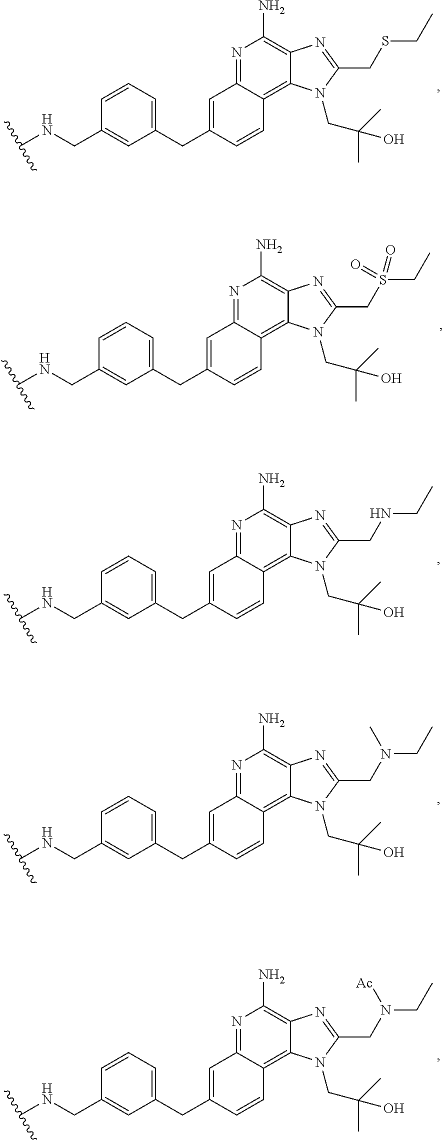 [SMILES: *NCc1cccc(Cc2ccc3c(c2)nc(N)c2nc(CSCC)n(CC(C)(C)O)c23)c1 ; *NCc1cccc(Cc2ccc3c(c2)nc(N)c2nc(CS(=O)(=O)CC)n(CC(C)(C)O)c23)c1 ; *NCc1cccc(Cc2ccc3c(c2)nc(N)c2nc(CNCC)n(CC(C)(C)O)c23)c1 ; *NCc1cccc(Cc2ccc3c(c2)nc(N)c2nc(CN(C)CC)n(CC(C)(C)O)c23)c1 ; *NCc1cccc(Cc2ccc3c(c2)nc(N)c2nc(CN(CC)C(C)=O)n(CC(C)(C)O)c23)c1]

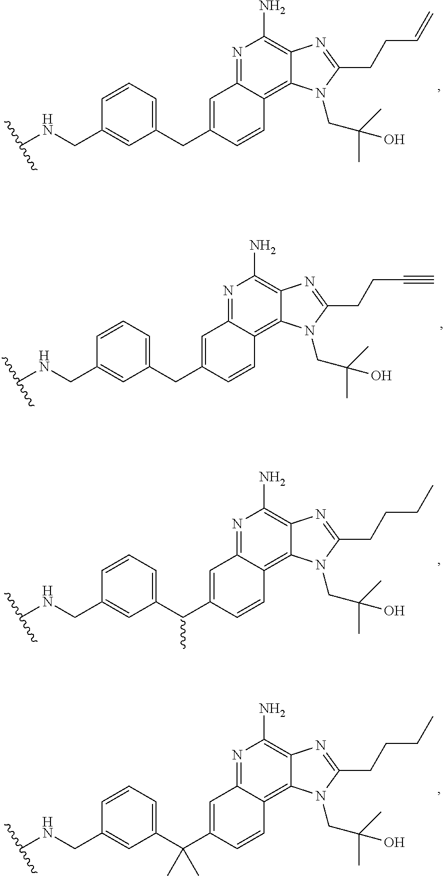 [SMILES: *NCc1cccc(Cc2ccc3c(c2)nc(N)c2nc(CCC=C)n(CC(C)(C)O)c23)c1 ; *NCc1cccc(Cc2ccc3c(c2)nc(N)c2nc(CCC#C)n(CC(C)(C)O)c23)c1 ; *NCc1cccc(C(C)c2ccc3c(c2)nc(N)c2nc(CCCC)n(CC(C)(C)O)c23)c1 ; *NCc1cccc(C(C)(C)c2ccc3c(c2)nc(N)c2nc(CCCC)n(CC(C)(C)O)c23)c1]

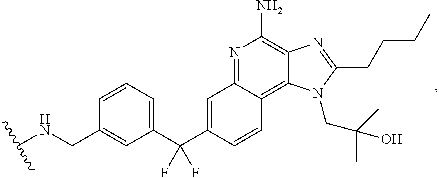 [SMILES: *NCc1cccc(C(F)(F)c2ccc3c(c2)nc(N)c2nc(CCCC)n(CC(C)(C)O)c23)c1]

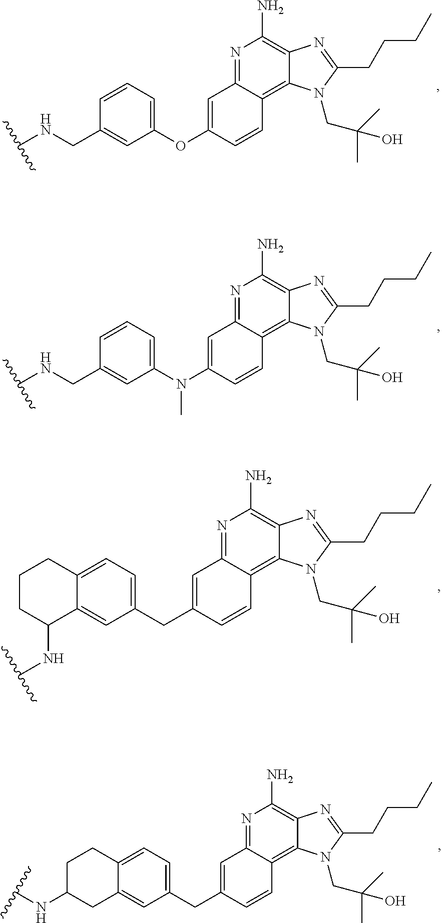 [SMILES: *NCc1cccc(Oc2ccc3c(c2)nc(N)c2nc(CCCC)n(CC(C)(C)O)c23)c1 ; *NCc1cccc(N(C)c2ccc3c(c2)nc(N)c2nc(CCCC)n(CC(C)(C)O)c23)c1 ; *NC1CCCc2ccc(Cc3ccc4c(c3)nc(N)c3nc(CCCC)n(CC(C)(C)O)c34)cc21 ; *NC1CCc2ccc(Cc3ccc4c(c3)nc(N)c3nc(CCCC)n(CC(C)(C)O)c34)cc2C1]

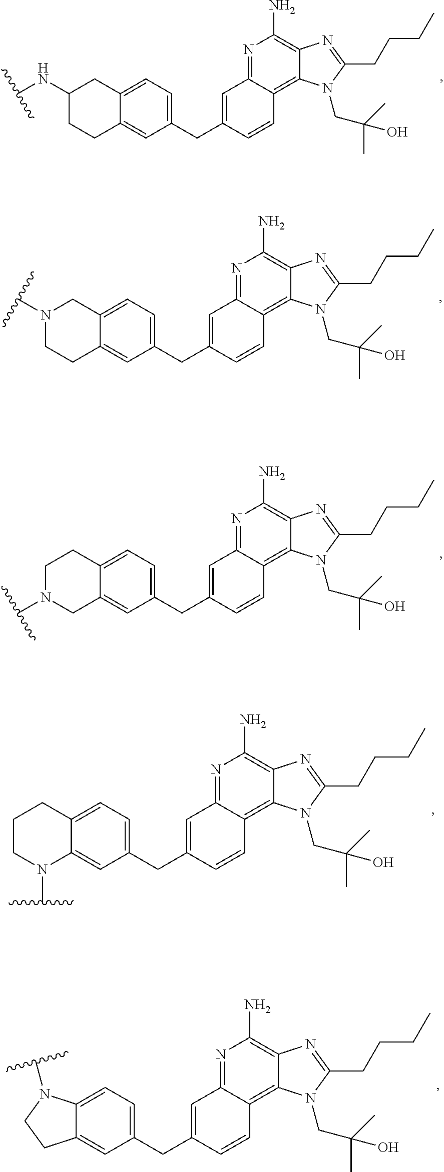 [SMILES: *NC1CCc2cc(Cc3ccc4c(c3)nc(N)c3nc(CCCC)n(CC(C)(C)O)c34)ccc2C1 ; *N1CCc2cc(Cc3ccc4c(c3)nc(N)c3nc(CCCC)n(CC(C)(C)O)c34)ccc2C1 ; *N1CCc2ccc(Cc3ccc4c(c3)nc(N)c3nc(CCCC)n(CC(C)(C)O)c34)cc2C1 ; *N1CCCc2ccc(Cc3ccc4c(c3)nc(N)c3nc(CCCC)n(CC(C)(C)O)c34)cc21 ; *N1CCc2cc(Cc3ccc4c(c3)nc(N)c3nc(CCCC)n(CC(C)(C)O)c34)ccc21]

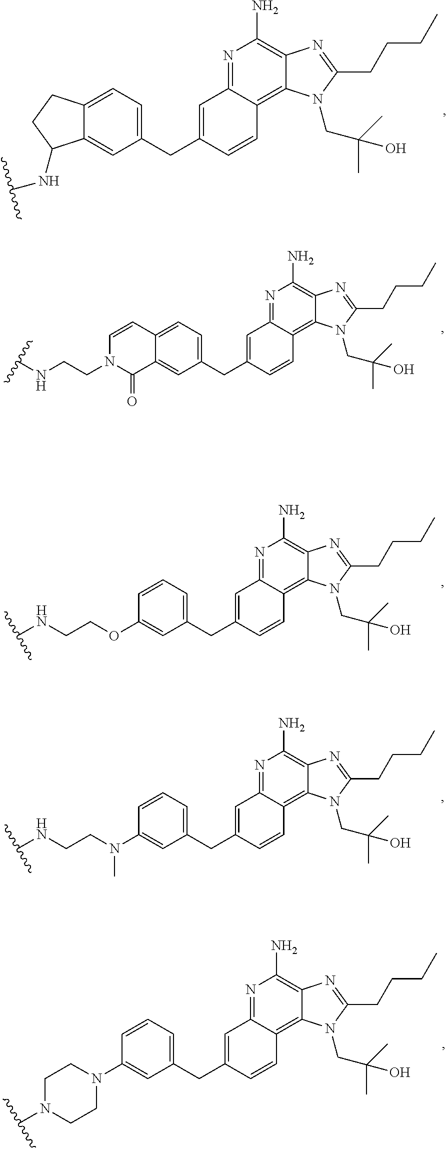 [SMILES: *NC1CCc2ccc(Cc3ccc4c(c3)nc(N)c3nc(CCCC)n(CC(C)(C)O)c34)cc21 ; *NCCn1ccc2ccc(Cc3ccc4c(c3)nc(N)c3nc(CCCC)n(CC(C)(C)O)c34)cc2c1=O ; *NCCOc1cccc(Cc2ccc3c(c2)nc(N)c2nc(CCCC)n(CC(C)(C)O)c23)c1 ; *NCCN(C)c1cccc(Cc2ccc3c(c2)nc(N)c2nc(CCCC)n(CC(C)(C)O)c23)c1 ; *N1CCN(c2cccc(Cc3ccc4c(c3)nc(N)c3nc(CCCC)n(CC(C)(C)O)c34)c2)CC1]

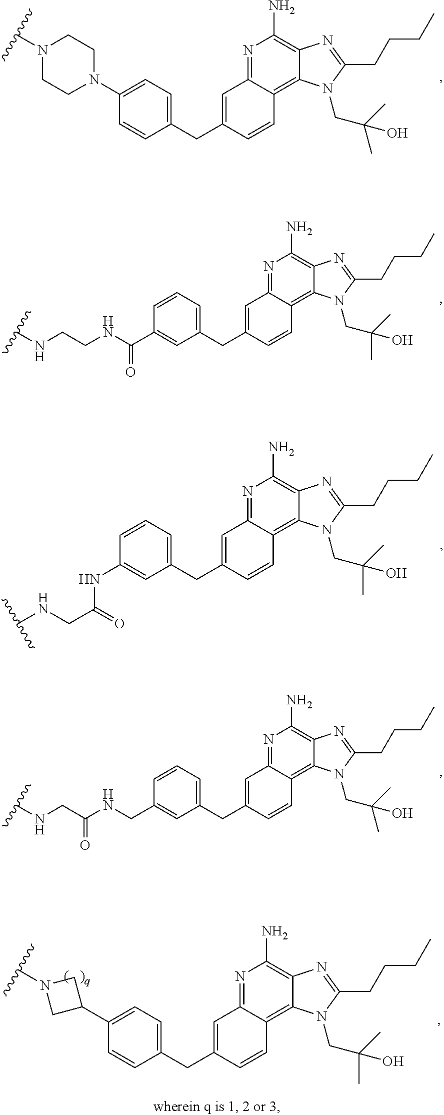

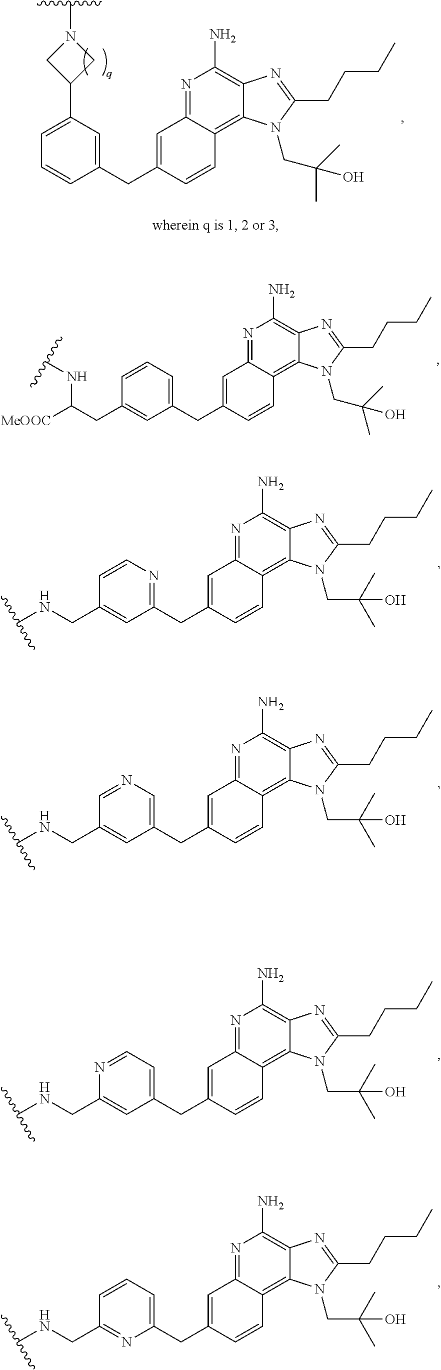

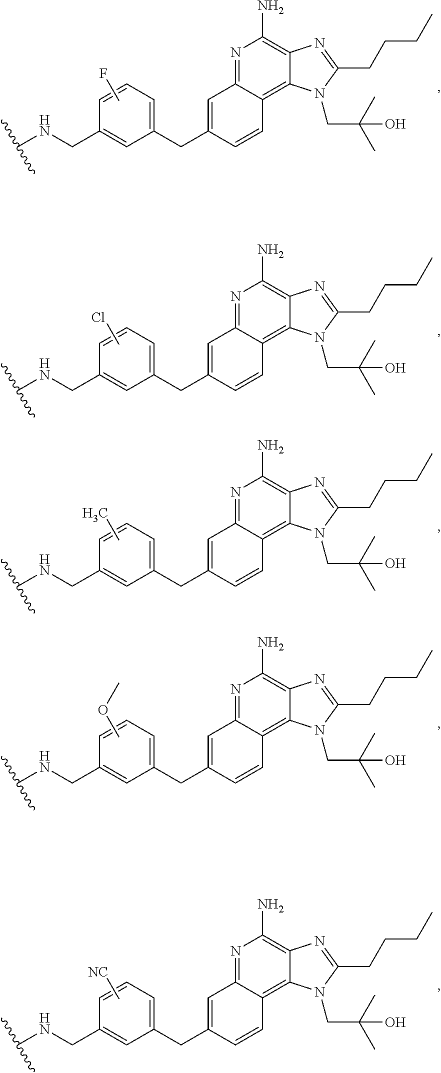

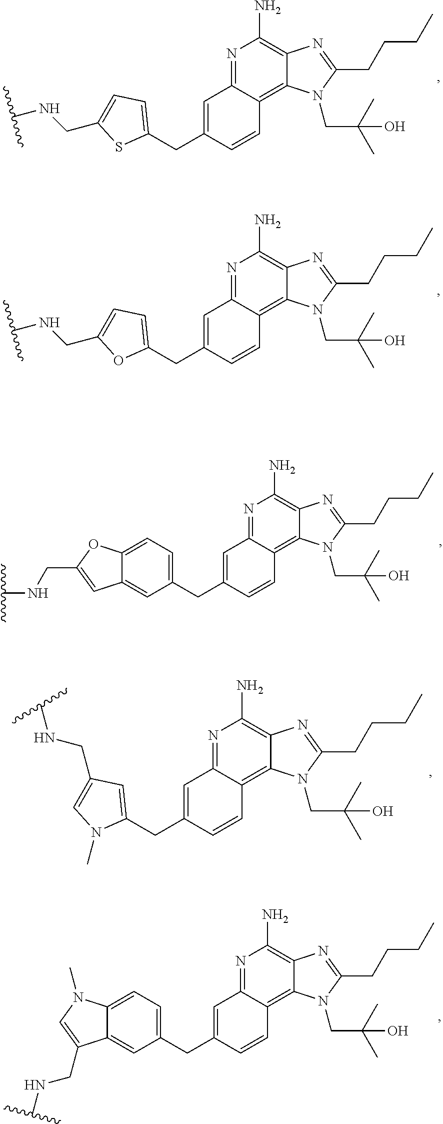 [SMILES: *NCc1ccc(Cc2ccc3c(c2)nc(N)c2nc(CCCC)n(CC(C)(C)O)c23)s1 ; *NCc1ccc(Cc2ccc3c(c2)nc(N)c2nc(CCCC)n(CC(C)(C)O)c23)o1 ; *NCc1cc2cc(Cc3ccc4c(c3)nc(N)c3nc(CCCC)n(CC(C)(C)O)c34)ccc2o1 ; *NCc1cc(Cc2ccc3c(c2)nc(N)c2nc(CCCC)n(CC(C)(C)O)c23)n(C)c1 ; *NCc1cn(C)c2ccc(Cc3ccc4c(c3)nc(N)c3nc(CCCC)n(CC(C)(C)O)c34)cc12]

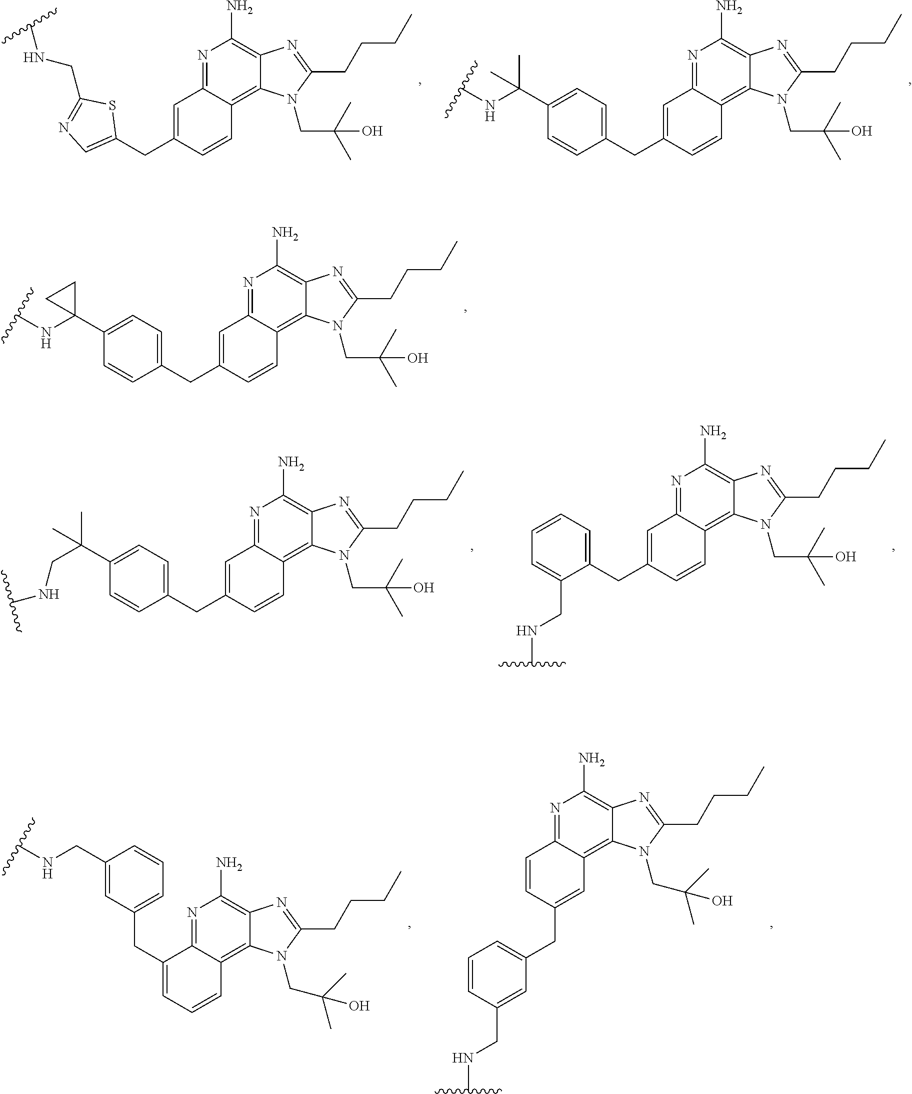

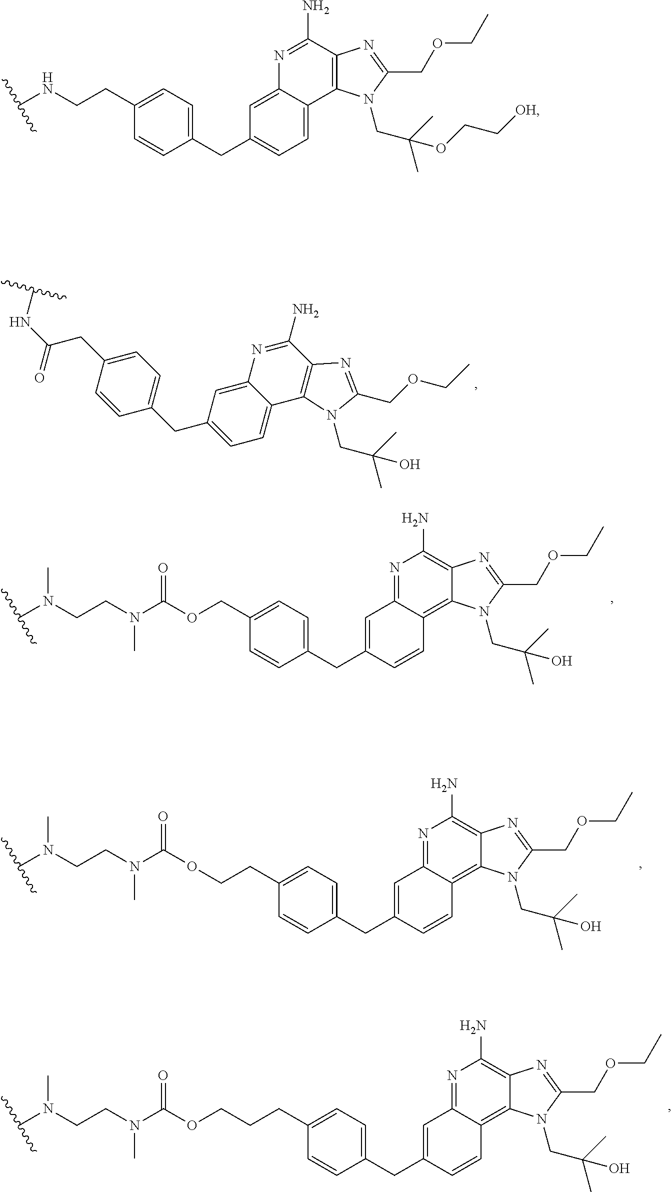 [SMILES: *NCCc1ccc(Cc2ccc3c(c2)nc(N)c2nc(COCC)n(CC(C)(C)OCCO)c23)cc1 ; *NC(=O)Cc1ccc(Cc2ccc3c(c2)nc(N)c2nc(COCC)n(CC(C)(C)O)c23)cc1 ; *N(C)CCN(C)C(=O)OCc1ccc(Cc2ccc3c(c2)nc(N)c2nc(COCC)n(CC(C)(C)O)c23)cc1 ; *N(C)CCN(C)C(=O)OCCc1ccc(Cc2ccc3c(c2)nc(N)c2nc(COCC)n(CC(C)(C)O)c23)cc1 ; *N(C)CCN(C)C(=O)OCCCc1ccc(Cc2ccc3c(c2)nc(N)c2nc(COCC)n(CC(C)(C)O)c23)cc1]

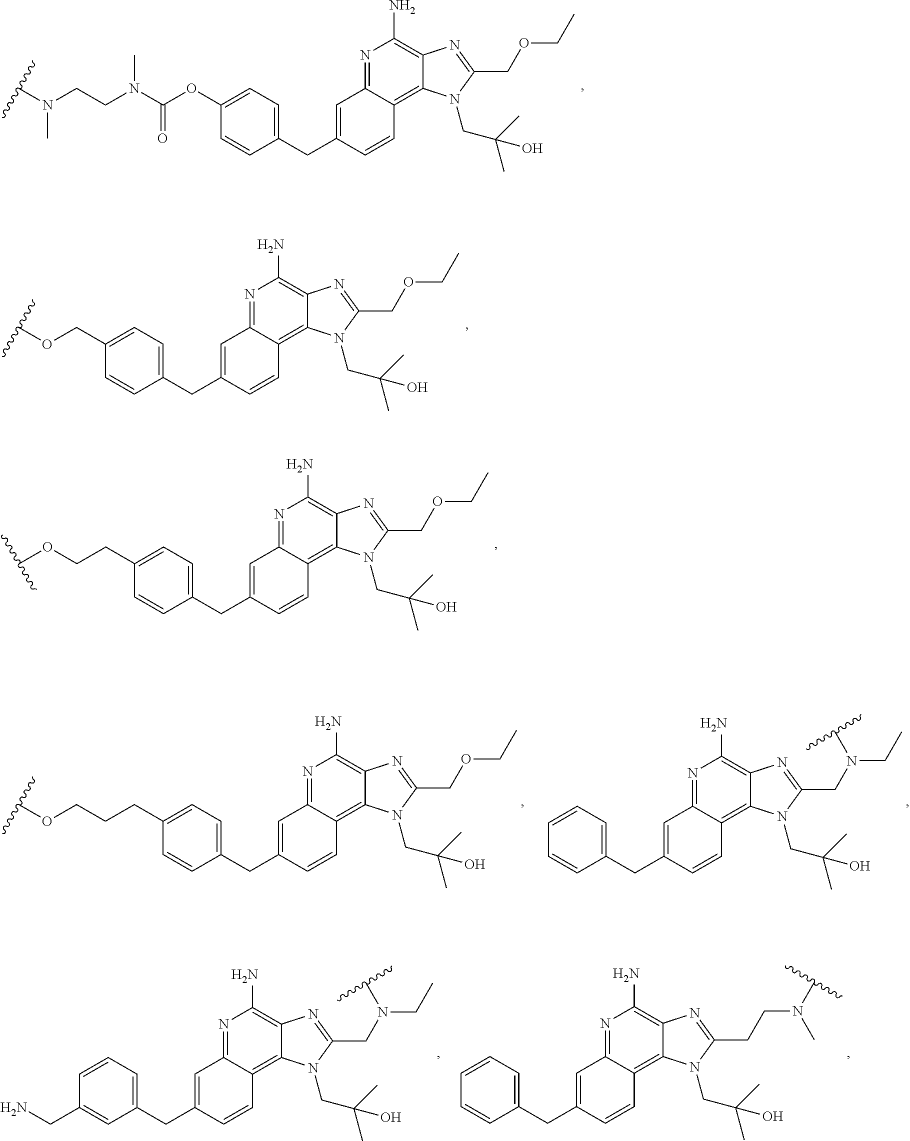

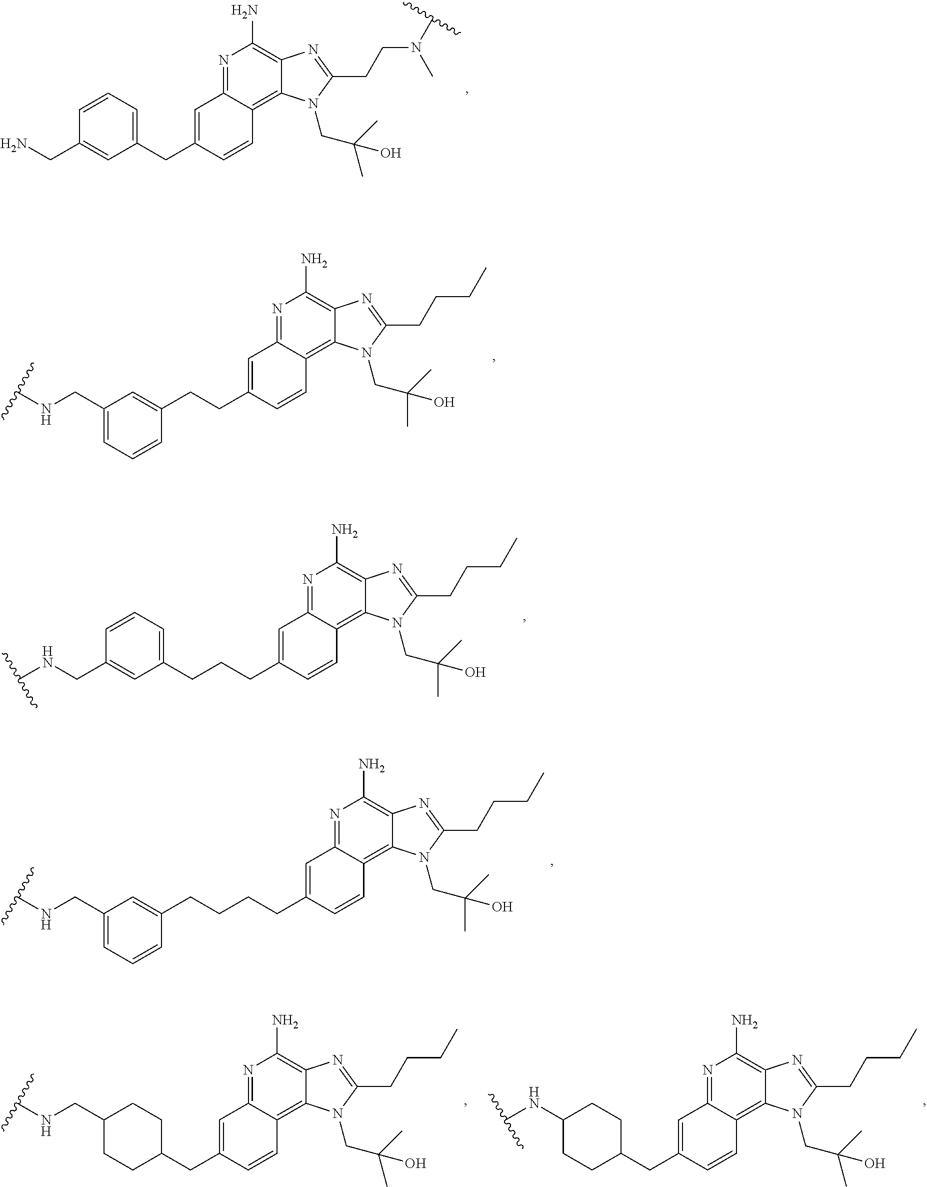 [SMILES: *N(C)CCc1nc2c(N)nc3cc(Cc4cccc(CN)c4)ccc3c2n1CC(C)(C)O ; *NCc1cccc(CCc2ccc3c(c2)nc(N)c2nc(CCCC)n(CC(C)(C)O)c23)c1 ; *NCc1cccc(CCCc2ccc3c(c2)nc(N)c2nc(CCCC)n(CC(C)(C)O)c23)c1 ; *NCc1cccc(CCCCc2ccc3c(c2)nc(N)c2nc(CCCC)n(CC(C)(C)O)c23)c1 ; *NCC1CCC(Cc2ccc3c(c2)nc(N)c2nc(CCCC)n(CC(C)(C)O)c23)CC1 ; *NC1CCC(Cc2ccc3c(c2)nc(N)c2nc(CCCC)n(CC(C)(C)O)c23)CC1]

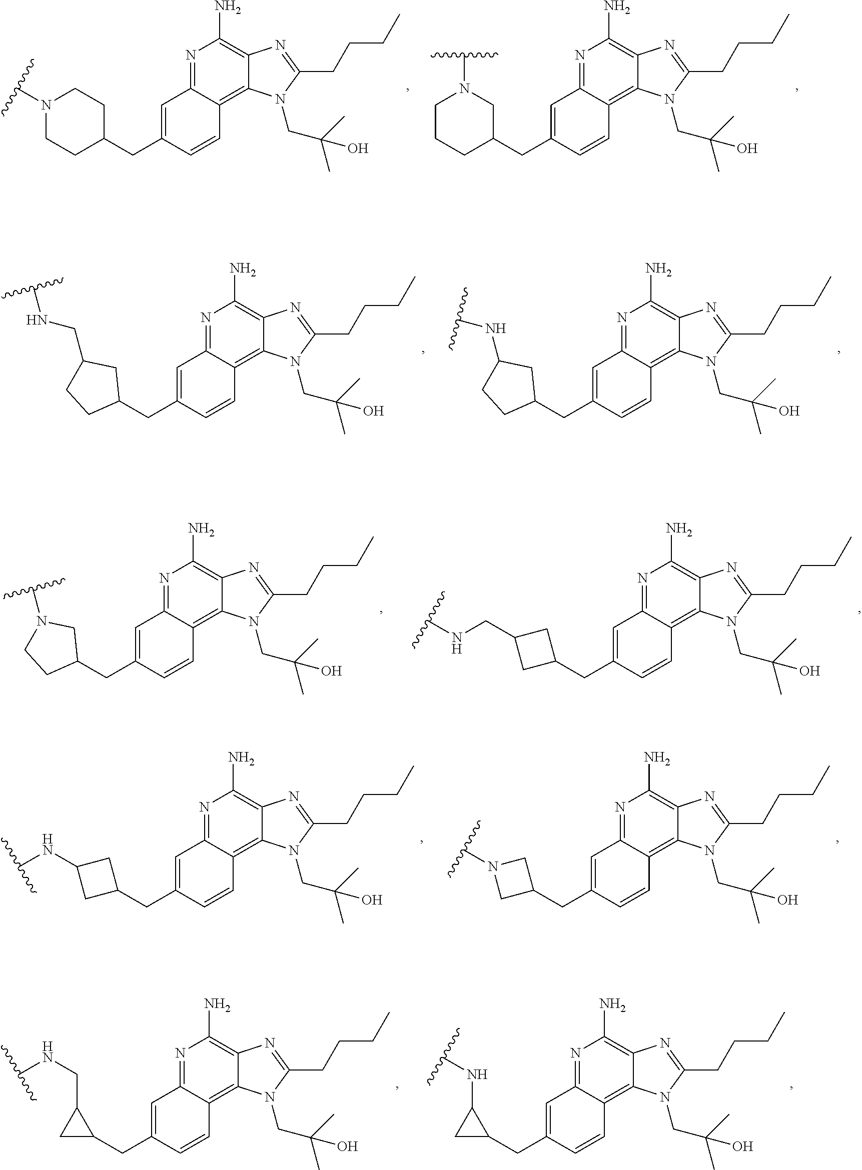

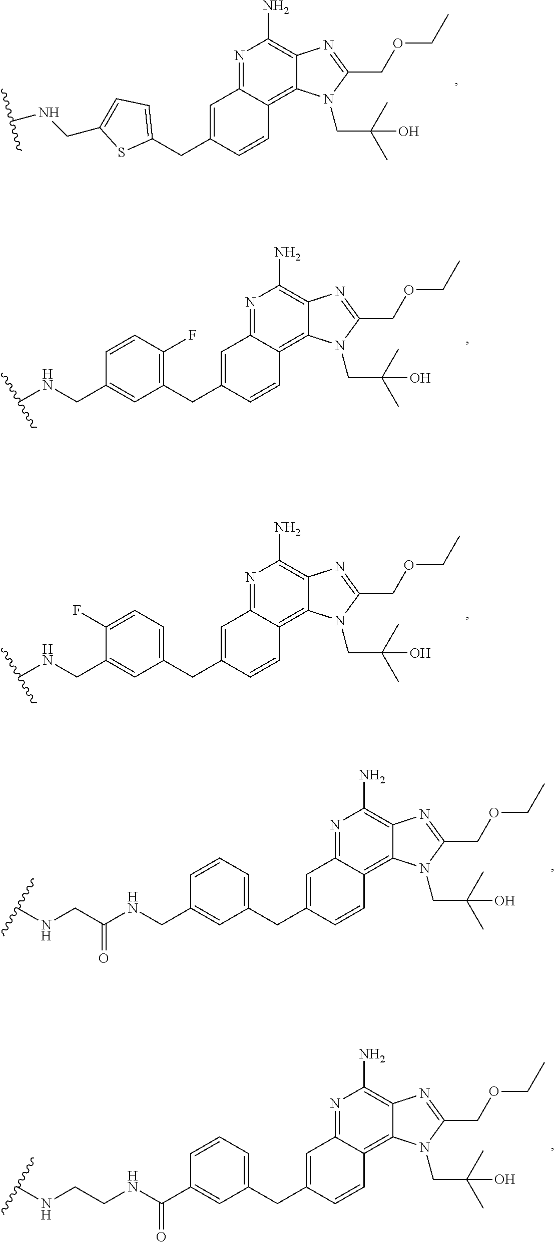 [SMILES: *NCc1ccc(Cc2ccc3c(c2)nc(N)c2nc(COCC)n(CC(C)(C)O)c23)s1 ; *NCc1ccc(F)c(Cc2ccc3c(c2)nc(N)c2nc(COCC)n(CC(C)(C)O)c23)c1 ; *NCc1cc(Cc2ccc3c(c2)nc(N)c2nc(COCC)n(CC(C)(C)O)c23)ccc1F ; *NCC(=O)NCc1cccc(Cc2ccc3c(c2)nc(N)c2nc(COCC)n(CC(C)(C)O)c23)c1 ; *NCCNC(=O)c1cccc(Cc2ccc3c(c2)nc(N)c2nc(COCC)n(CC(C)(C)O)c23)c1]

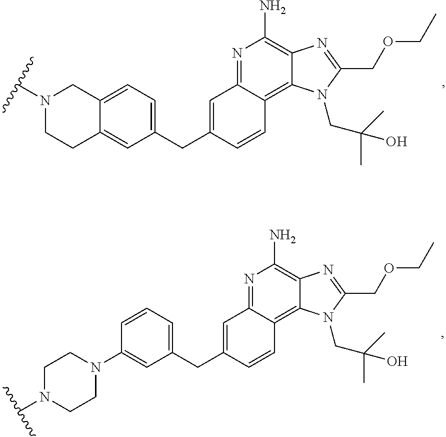 [SMILES: *N1CCc2cc(Cc3ccc4c(c3)nc(N)c3nc(COCC)n(CC(C)(C)O)c34)ccc2C1 ; *N1CCN(c2cccc(Cc3ccc4c(c3)nc(N)c3nc(COCC)n(CC(C)(C)O)c34)c2)CC1]

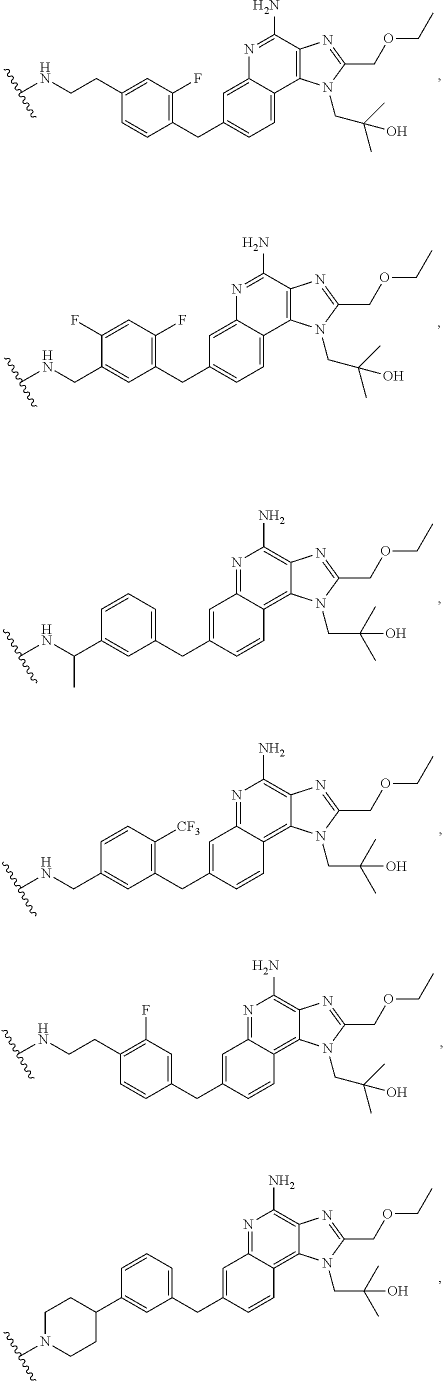 [SMILES: *NCCc1ccc(Cc2ccc3c(c2)nc(N)c2nc(COCC)n(CC(C)(C)O)c23)c(F)c1 ; *NCc1cc(Cc2ccc3c(c2)nc(N)c2nc(COCC)n(CC(C)(C)O)c23)c(F)cc1F ; *NC(C)c1cccc(Cc2ccc3c(c2)nc(N)c2nc(COCC)n(CC(C)(C)O)c23)c1 ; *NCc1ccc(C(F)(F)F)c(Cc2ccc3c(c2)nc(N)c2nc(COCC)n(CC(C)(C)O)c23)c1 ; *NCCc1ccc(Cc2ccc3c(c2)nc(N)c2nc(COCC)n(CC(C)(C)O)c23)cc1F ; *N1CCC(c2cccc(Cc3ccc4c(c3)nc(N)c3nc(COCC)n(CC(C)(C)O)c34)c2)CC1]

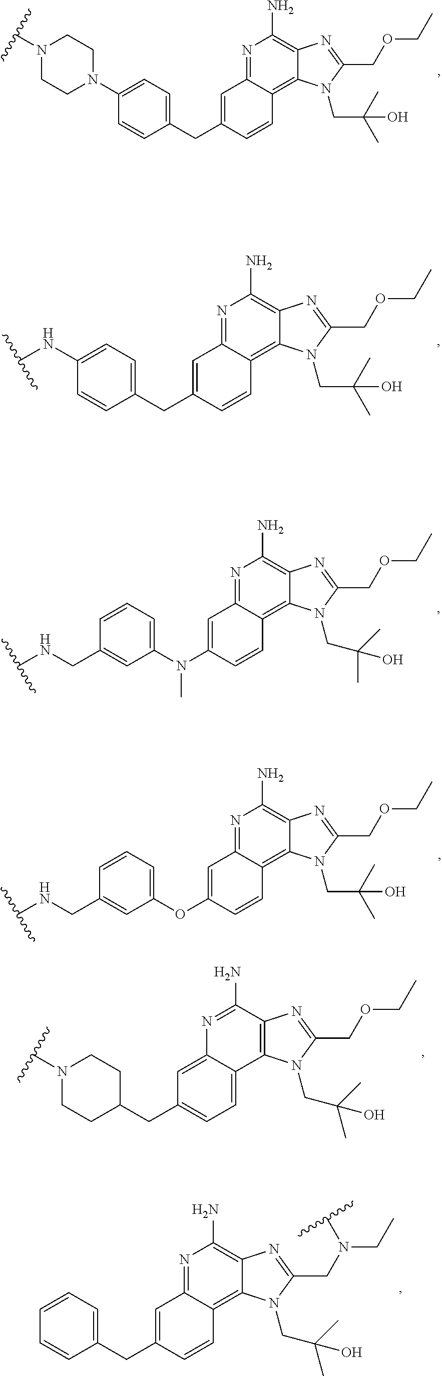 [SMILES: *N1CCN(c2ccc(Cc3ccc4c(c3)nc(N)c3nc(COCC)n(CC(C)(C)O)c34)cc2)CC1 ; *Nc1ccc(Cc2ccc3c(c2)nc(N)c2nc(COCC)n(CC(C)(C)O)c23)cc1 ; *NCc1cccc(N(C)c2ccc3c(c2)nc(N)c2nc(COCC)n(CC(C)(C)O)c23)c1 ; *NCc1cccc(Oc2ccc3c(c2)nc(N)c2nc(COCC)n(CC(C)(C)O)c23)c1 ; *N1CCC(Cc2ccc3c(c2)nc(N)c2nc(COCC)n(CC(C)(C)O)c23)CC1 ; *N(CC)Cc1nc2c(N)nc3cc(Cc4ccccc4)ccc3c2n1CC(C)(C)O]

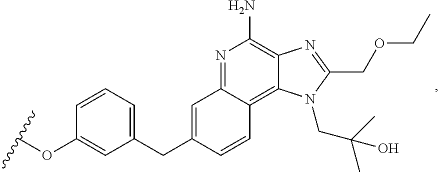 [SMILES: *Oc1cccc(Cc2ccc3c(c2)nc(N)c2nc(COCC)n(CC(C)(C)O)c23)c1]

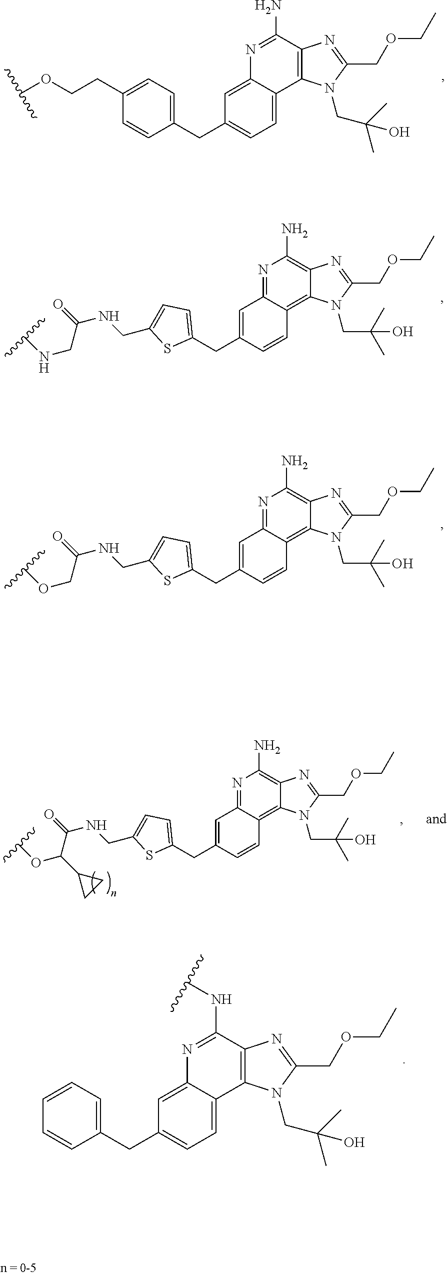

In some embodiments, L has Formula (III):

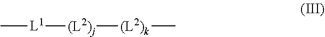

wherein
L¹ is a stretcher unit covalently attached to the targeting moiety;
L² is an optional peptide unit of two to twelve amino acid residues,
L³ is an optional spacer unit covalently attached to the payload unit, and
j and k are independently selected from 0 and 1.

In certain embodiments, L¹ is selected from the group consisting of:

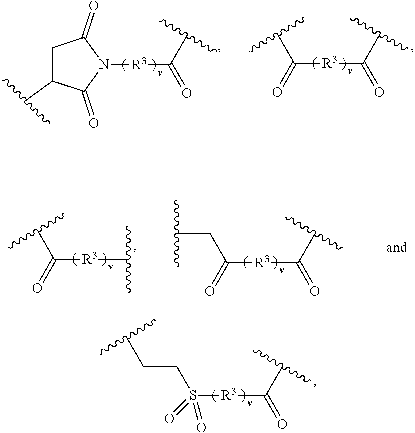

wherein each R³ is independently selected from the group consisting of alkyl, heteroalkyl, cycloalkyl, heterocyclyl, aryl, heteroaryl, alkylaryl, arylalkyl, alkylcycloalkyl, cycloalkylalkyl, alkylheterocyclyl, heterocyclylalkyl, -alkyl-C(O)N(R^{a})-alkyl-N(R^{a}), —N(R^{a})-alkyl-, and —(CH₂CH₂O)ᵣ—CH₂—, wherein R^{a} is H or alkyl, and r is an integer ranging from 1 to 10, v is an integer ranging from 0 to 5.

In certain embodiments, each R³ is independently selected from the group consisting of C₁₋₁₀ alkyl, C₁₋₈ heteroalkyl, C₃₋₈ cycloalkyl, C₃₋₈ heterocyclyl, aryl, heteroaryl, (C₁₋₁₀ alkyl)aryl, aryl(C₁₋₁₀ alkyl), (C₁₋₁₀ alkyl)(C₃₋₈ cycloalkyl), (C₃₋₈ cycloalkyl)(C₁₋₁₀ alkyl), (C₁₋₁₀ alkyl)(C₃₋₈ heterocyclyl), (C₃₋₈ heterocyclyl)(C₁₋₁₀ alkyl), —(C₂₋₆ alkyl)-C(O)N(R^{a})—(C₂₋₆ alkyl)-N(R^{a}), —N(R^{a})—(C₂₋₆ alkyl)-, and —(CH₂CH₂O), —CH₂—, wherein R^{a} is H or C₁₋₆ alkyl.

In certain embodiments, v is 1 and R³ is (CH₂)₅.

In some embodiments, j is 0 and k is 0.

In some embodiments, j is 0 and k is 1.

In some embodiments, L¹ has the formula:

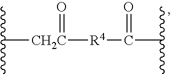

wherein R⁴ is selected from the group consisting of alkyl, -alkyl-O—, —N(R^{a})-alkyl-N(R^{a})—, —N(R^{a})-alkyl-, and (CH₂CH₂O)ᵣ—CH₂; wherein R^{a} is H or alkyl, and r is an integer ranging from 1 to 10.

In certain embodiments, L¹ has the formula:

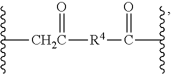

wherein R⁴ is selected from the group consisting of C₁₋₁₀ alkyl, —(C₁₋₁₀ alkyl)-O—, —N(R^{a})—(C₂₋₆ alkyl)-N(R^{a})—, —N(R^{a})—(C₂₋₆ alkyl)-, and —(CH₂CH₂O)ᵣ—CH₂—, wherein R^{a} is H or C₁₋₆ alkyl.

In some embodiments, L¹ has the formula:

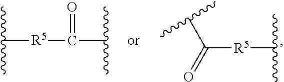

wherein R⁵ is selected from alkyl, -alkyl-O—, aryl, —N(R^{a})-alkyl- or —(CH₂CH₂O)ᵣ—CH₂—; wherein R^{a} is H or alkyl, and r is an integer ranging from 1 to 10.

In certain embodiments, L¹ has the formula:

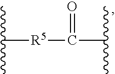

wherein R⁵ is selected from the group consisting of C₁₋₁₀ alkyl, —(C₁₋₁₀ alkyl)-O—, —N(R^{a})—(C₂₋₆ alkyl)— or —(CH₂CH₂O)ᵣ—CH₂—; wherein R^{a} is H or C₁₋₆ alkyl.

In certain embodiments, L¹ has the formula:

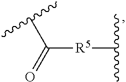

herein R⁵ is selected from the group consisting of C₁₋₁₀ alkyl, —(C₁₋₁₀ alkyl)-O—, —N(R^{a})—(C₂₋₆ alkyl)— or —(CH₂CH₂O), —CH₂—; wherein R^{a} is H or C₁₋₆ alkyl.

In certain embodiments, L forms a thioether bond with a cysteine amino acid of the targeting moiety, L¹ has the formula:

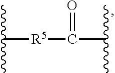

and R⁵ is —(C₂₋₆ alkyl)-O—, wherein the C₂₋₆ alkyl is optionally substituted with F, OH, O(C₁₋₆ alkyl), NH₂, NHCH₃, N(CH₃)₂, OP(O)₃H₂, and C₁₋₆ alkyl, wherein the C₁₋₆ alkyl is optionally substituted with one or more F.

In certain embodiments, L forms an amide bond with a lysine amino acid of the targeting moiety, L¹ has the formula:

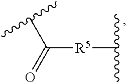

and R⁵ is (C₂₋₆ alkyl)-O—, wherein the C₂₋₆ alkyl is optionally substituted with F, OH, O(C₁₋₆ alkyl), NH₂, NHCH₃, N(CH₃)₂, OP(O)₃H₂, and C₁₋₆ alkyl, wherein the C₁₋₆ alkyl is optionally substituted with one or more F.

In some embodiments, j is 1 and k is 1.

In some embodiments, j is 1 and L² comprises two or twelve amino acid residues independently selected from glycine, alanine, phenylalanine, lysine, arginine, valine, and citrulline.

In certain embodiments, L² is valine-citrulline.

In some embodiments, k is 1 and L³ comprises para-aminobenzyl or para-aminobenzyloxycarbonyl.

In certain embodiments, there is provided a conjugate compound having the formula:

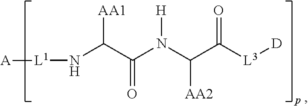

wherein AA1 and AA2 are independently selected from an amino acid side chain; p is an integer from 1 to 8.

In certain embodiments, the amino acid side chain is independently selected from H, —CH₃, —CH₂(C₆H₅), —CH₂CH₂CH₂CH₂NH₂, —CH₂CH₂CH₂NHC(NH)NH₂, —CHCH(CH₃)CH₃, and —CH₂CH₂CH₂NHC(O)NH₂.

In certain embodiments, there is provided a conjugate compound having the formula:

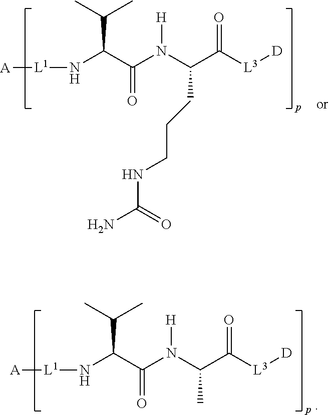

In certain embodiments, there is provided a conjugate compound having the formula:

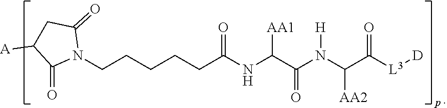

In certain embodiments, there is provided a conjugate compound having the formula:

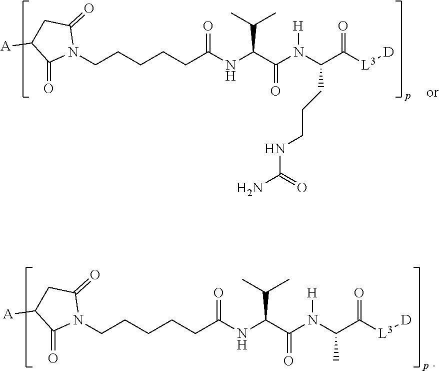

In certain embodiments, there is provided a conjugate compound having the formula:

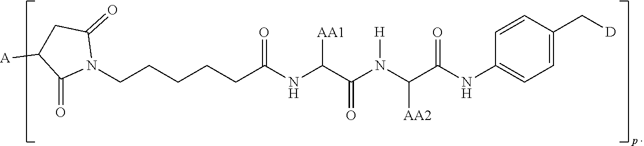

In certain embodiments, there is provided a conjugate compound having the formula:

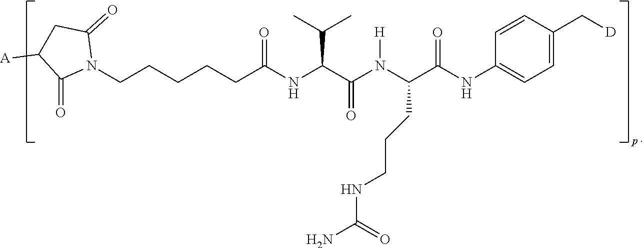

In certain embodiments, there is provided a conjugate compound having the formula:

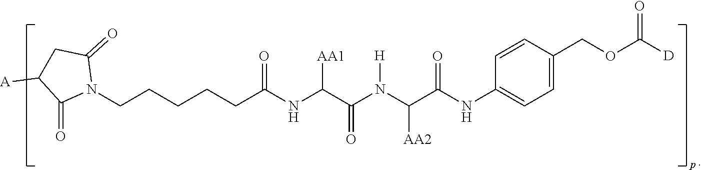

In certain embodiments, there is provided a conjugate compound having the formula:

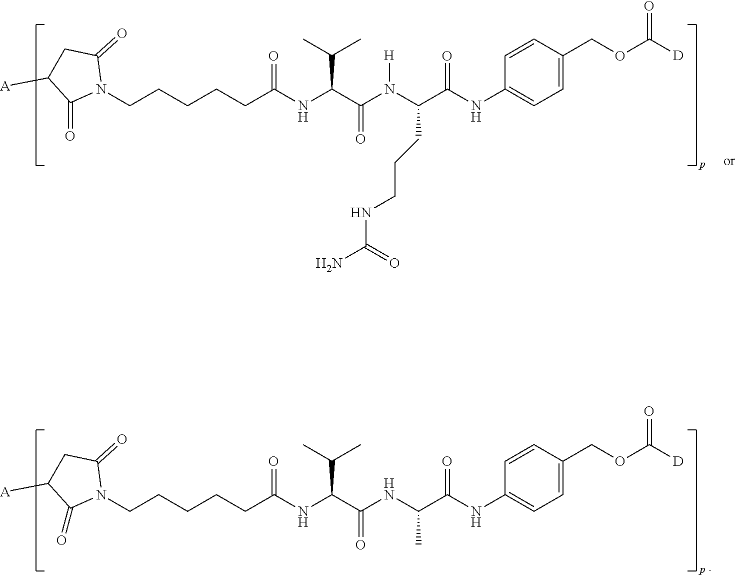

In certain embodiments, there is provided a conjugate compound selected from the group consisting of:

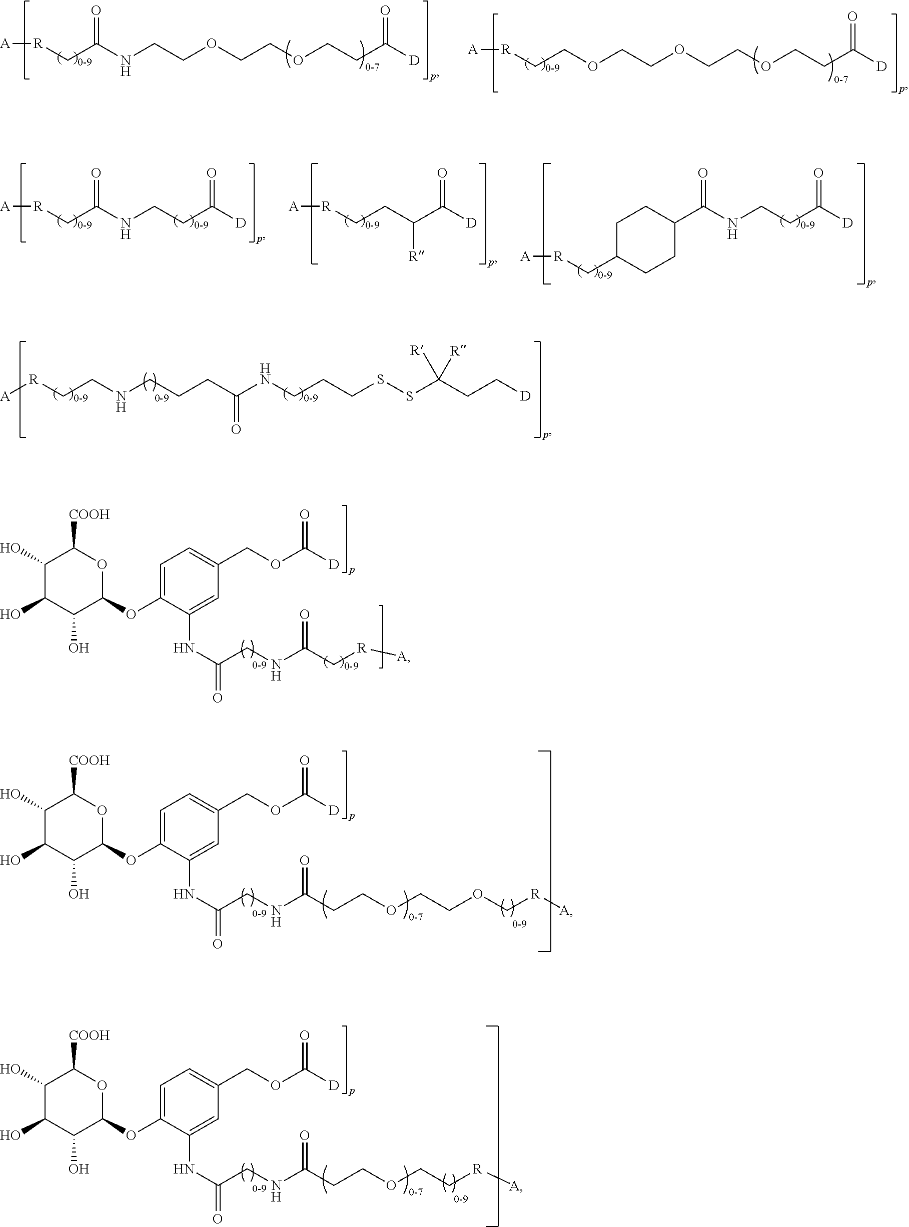

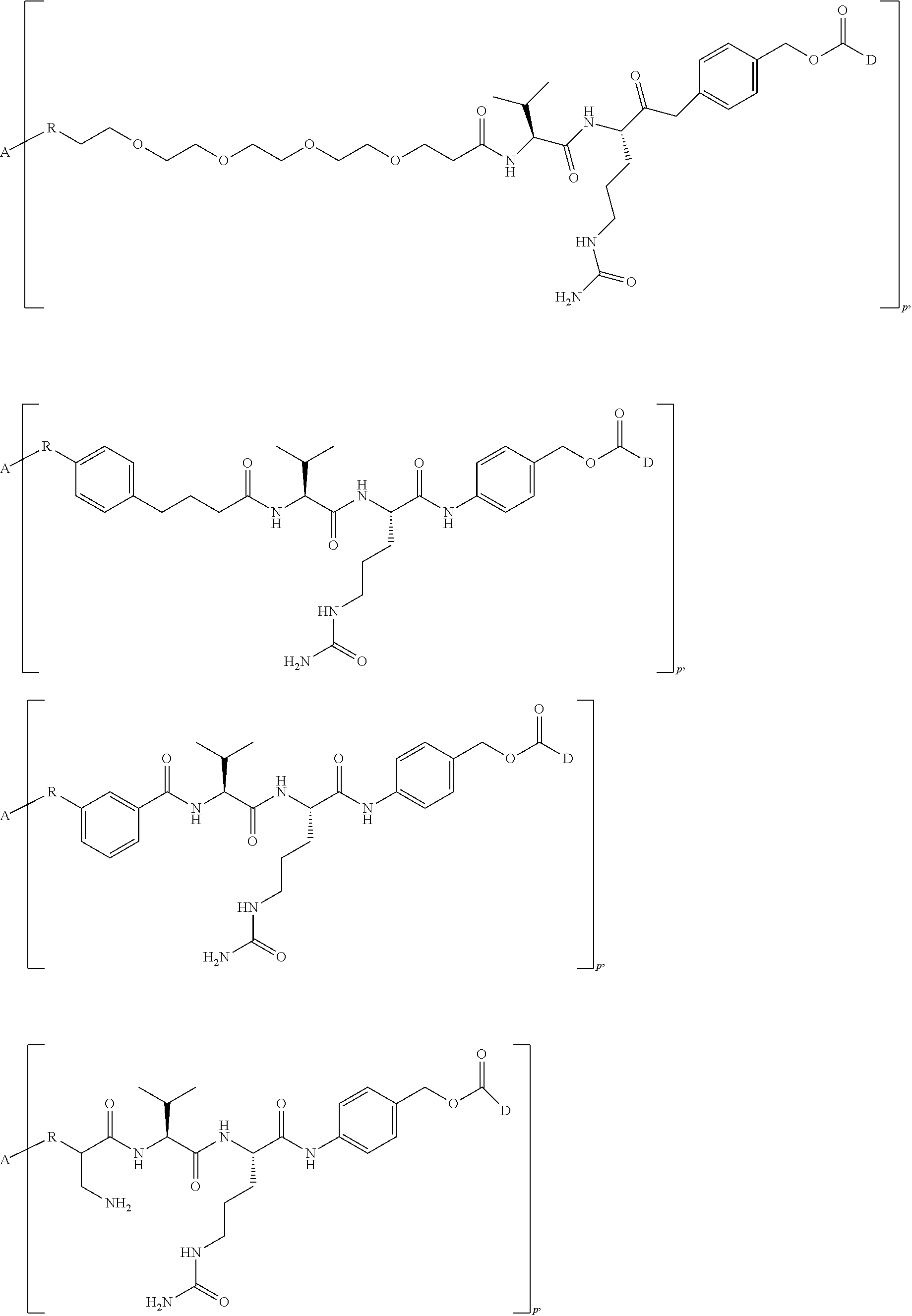

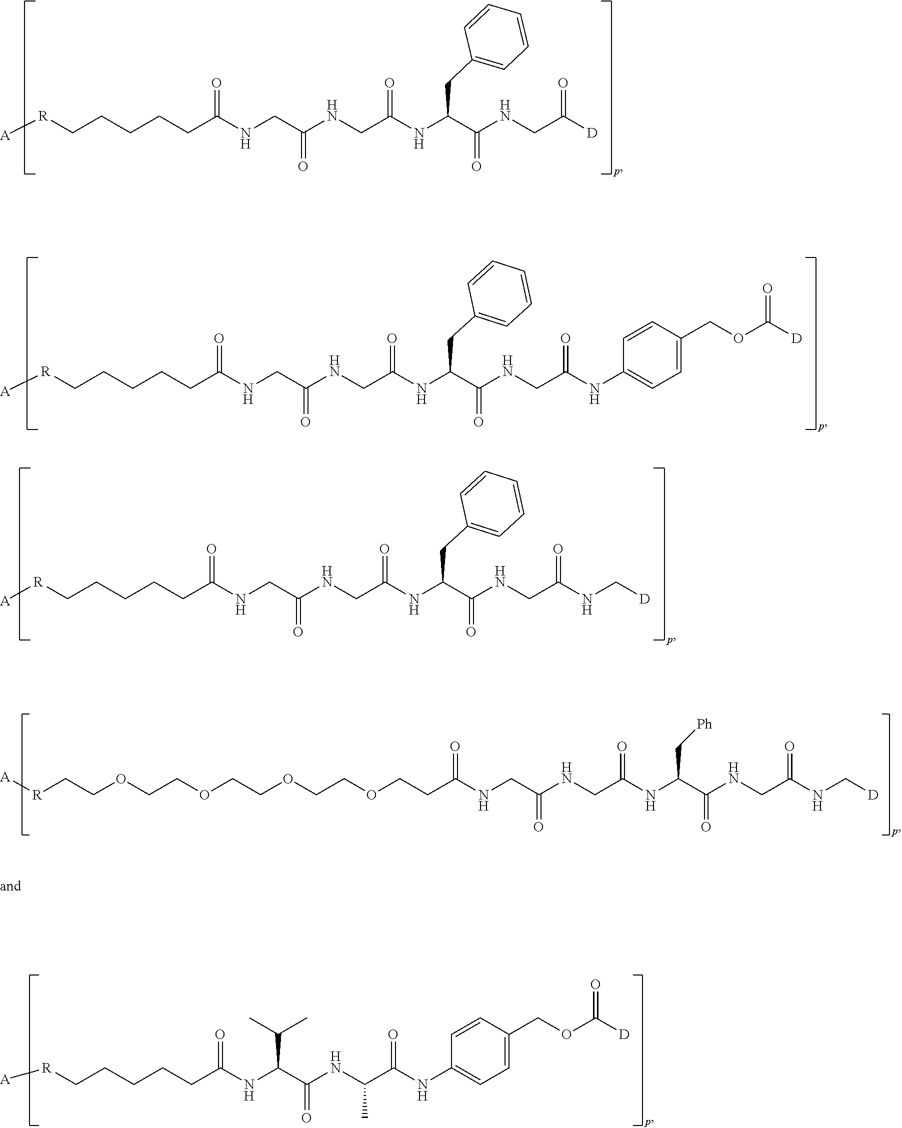

wherein R is selected from the group consisting of:

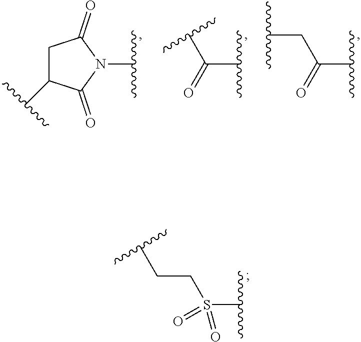

wherein each of R′ and R″ is independently hydrogen or methyl.

In certain embodiments, there is provided a conjugate compound selected from the group consisting of:

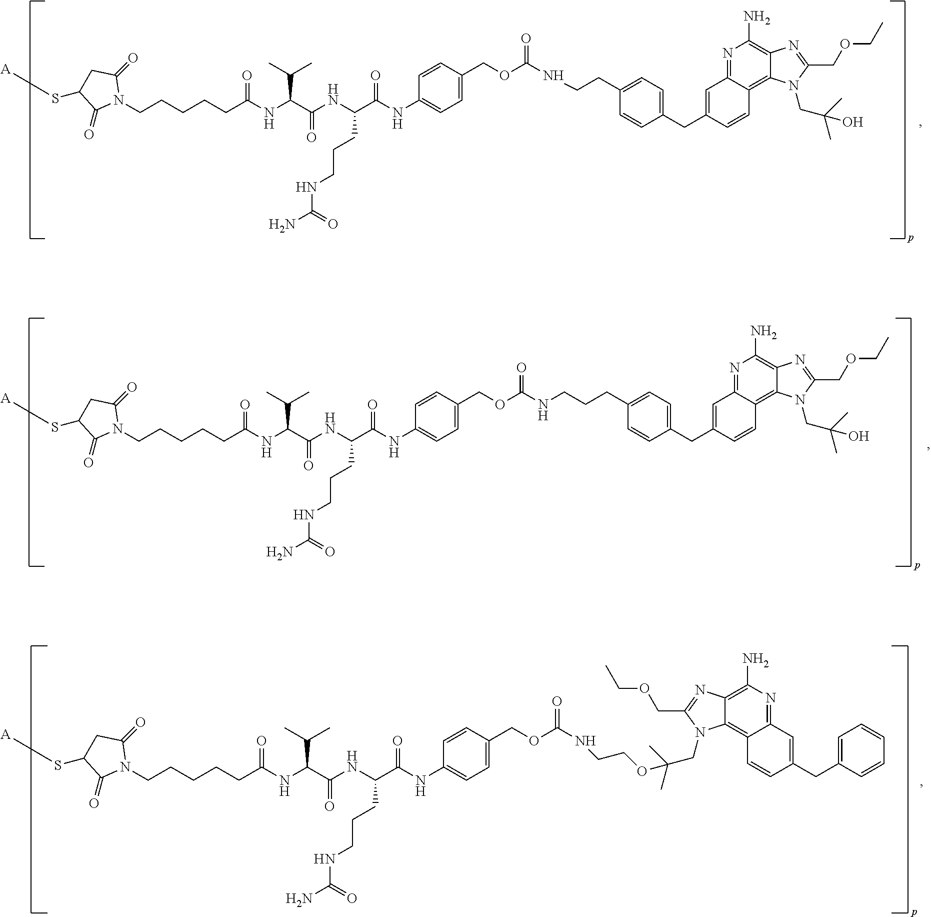

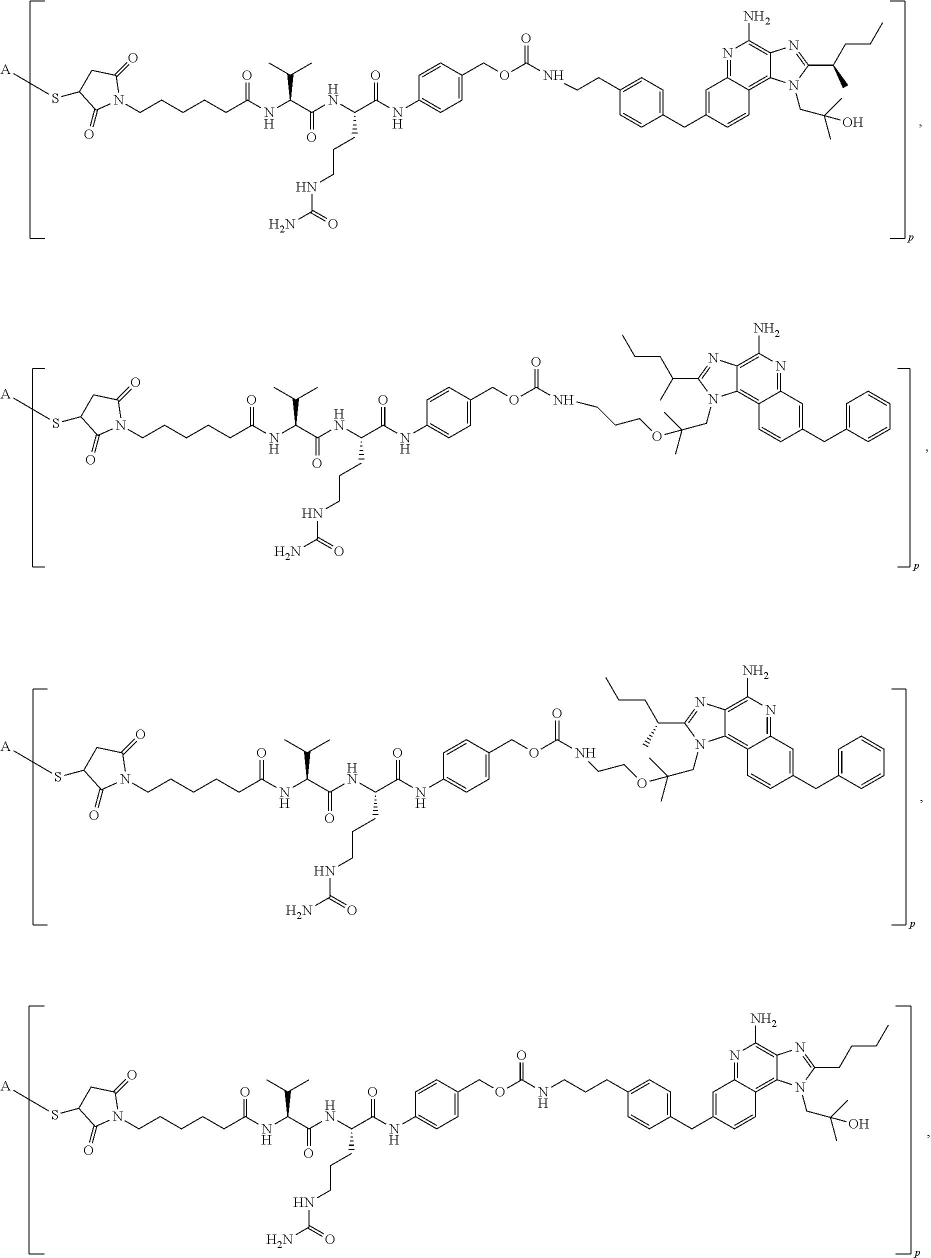

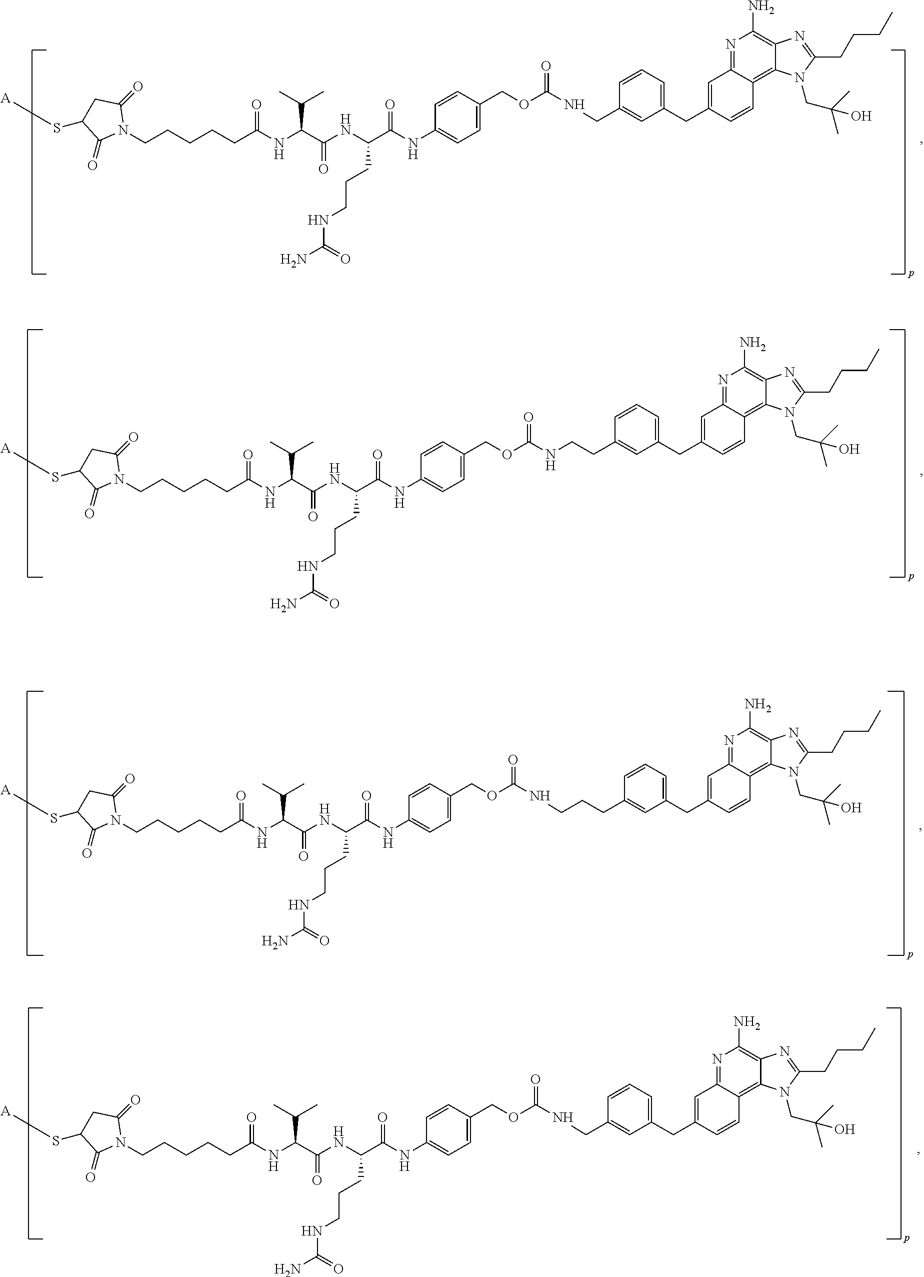

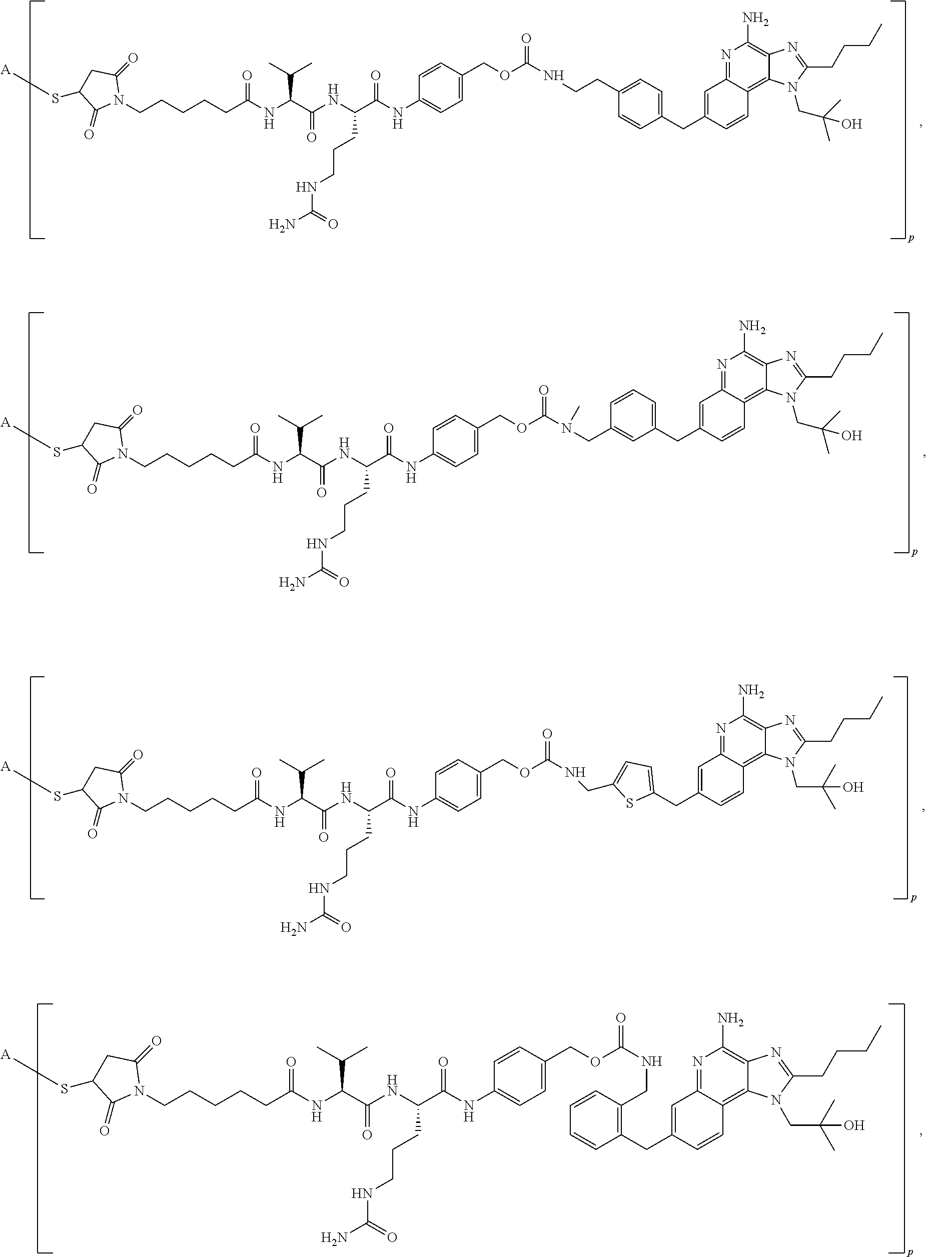

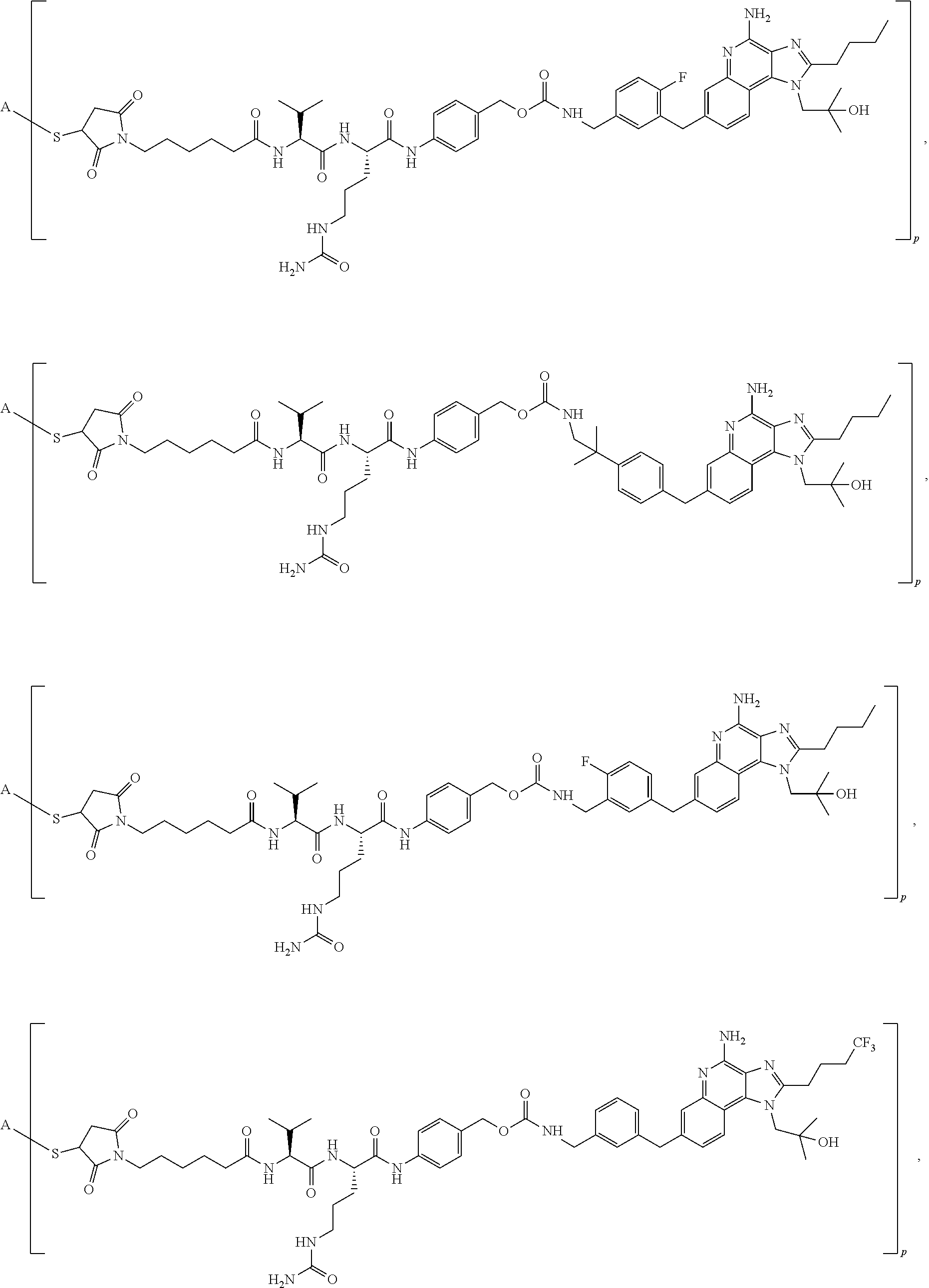

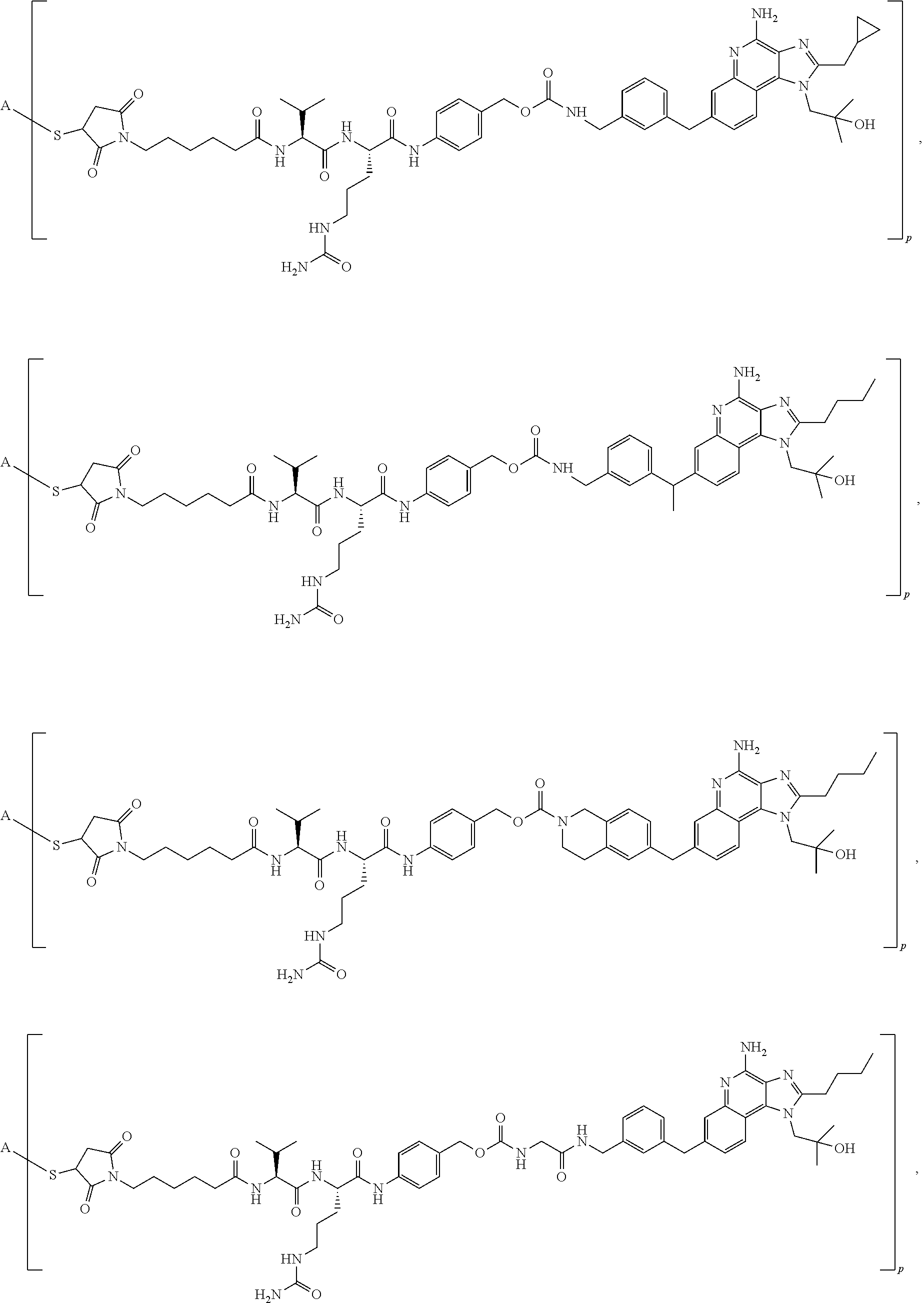

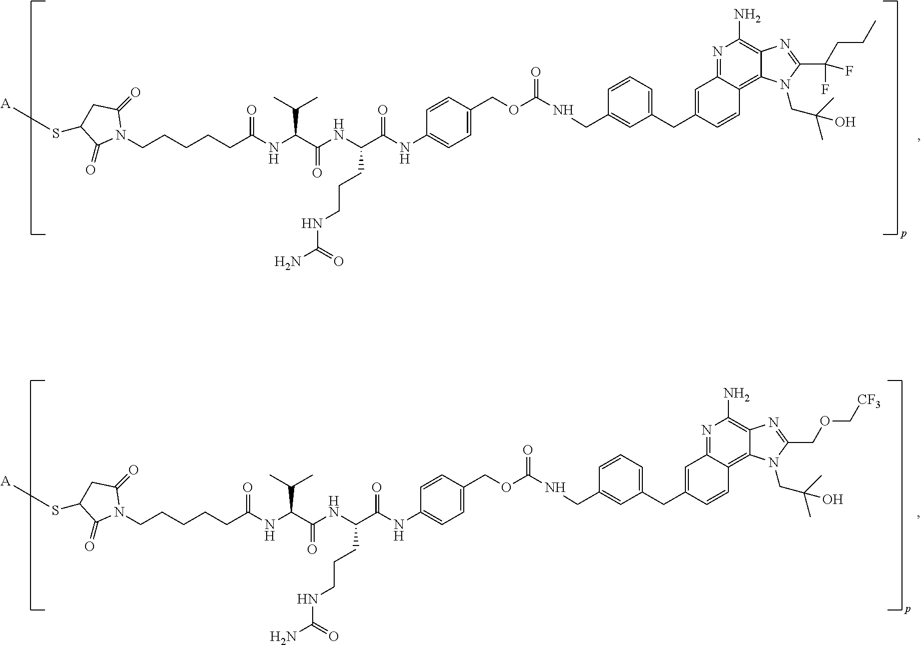

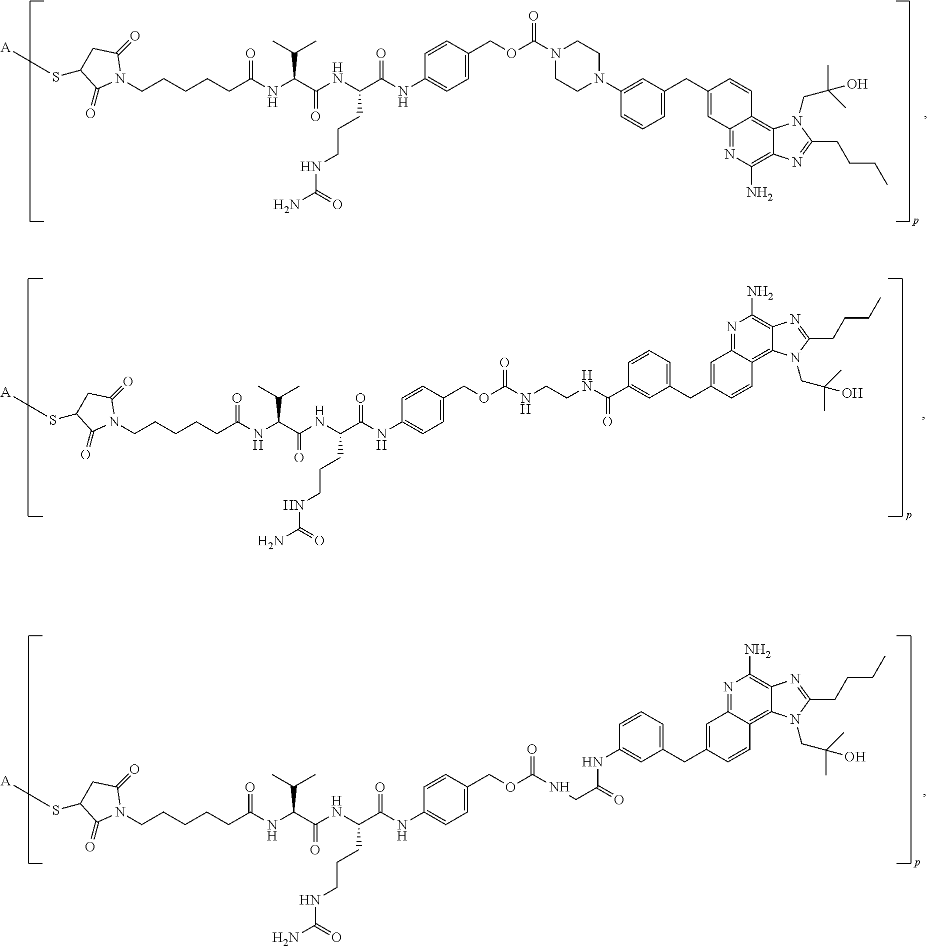

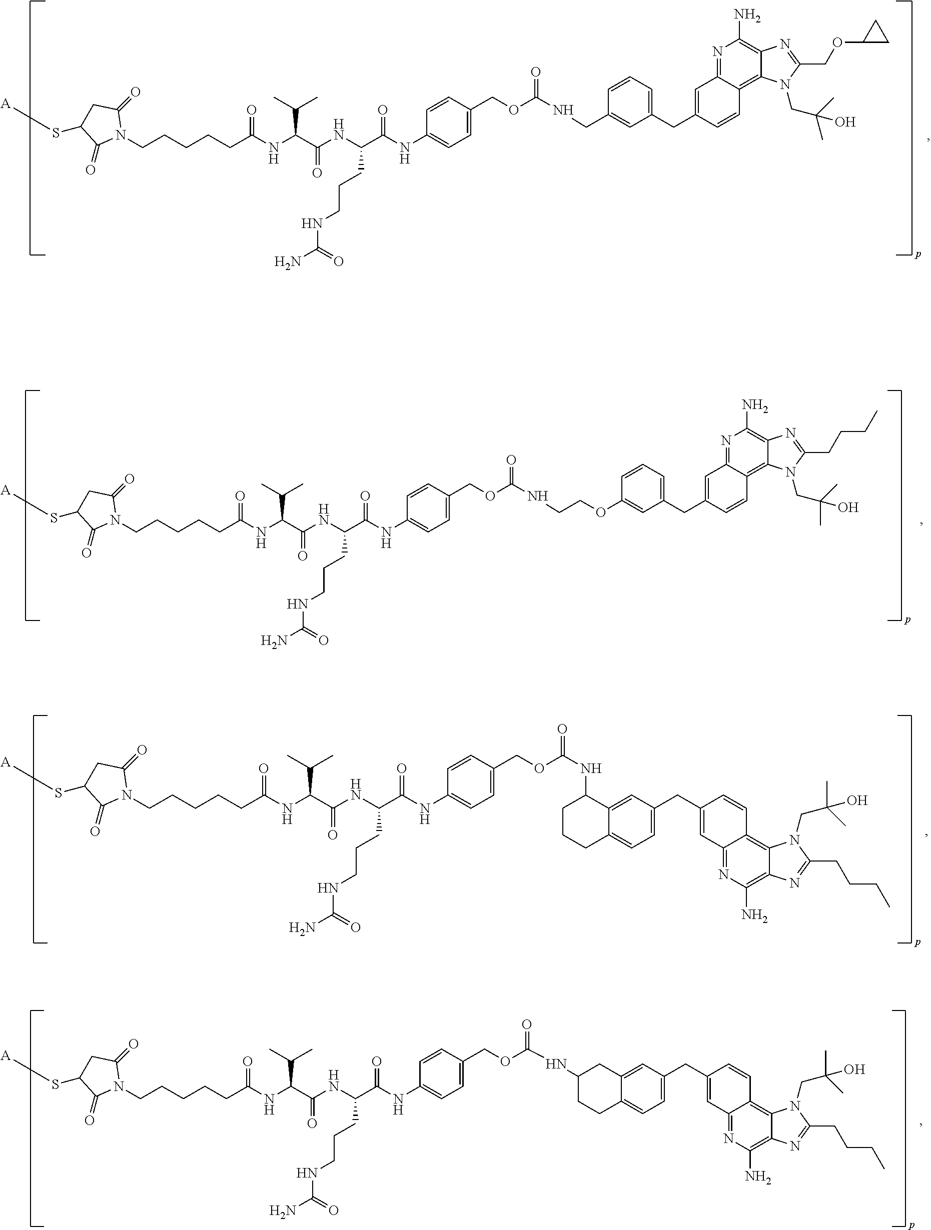

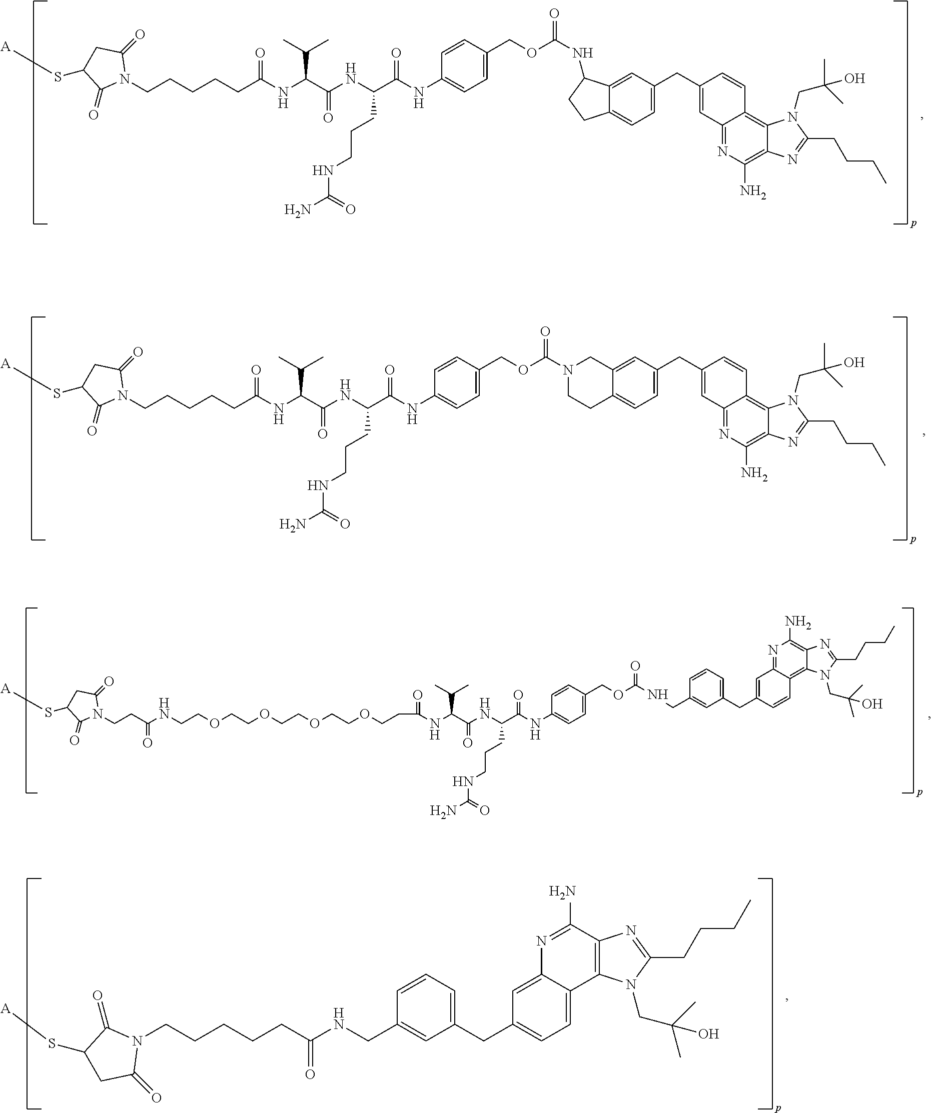

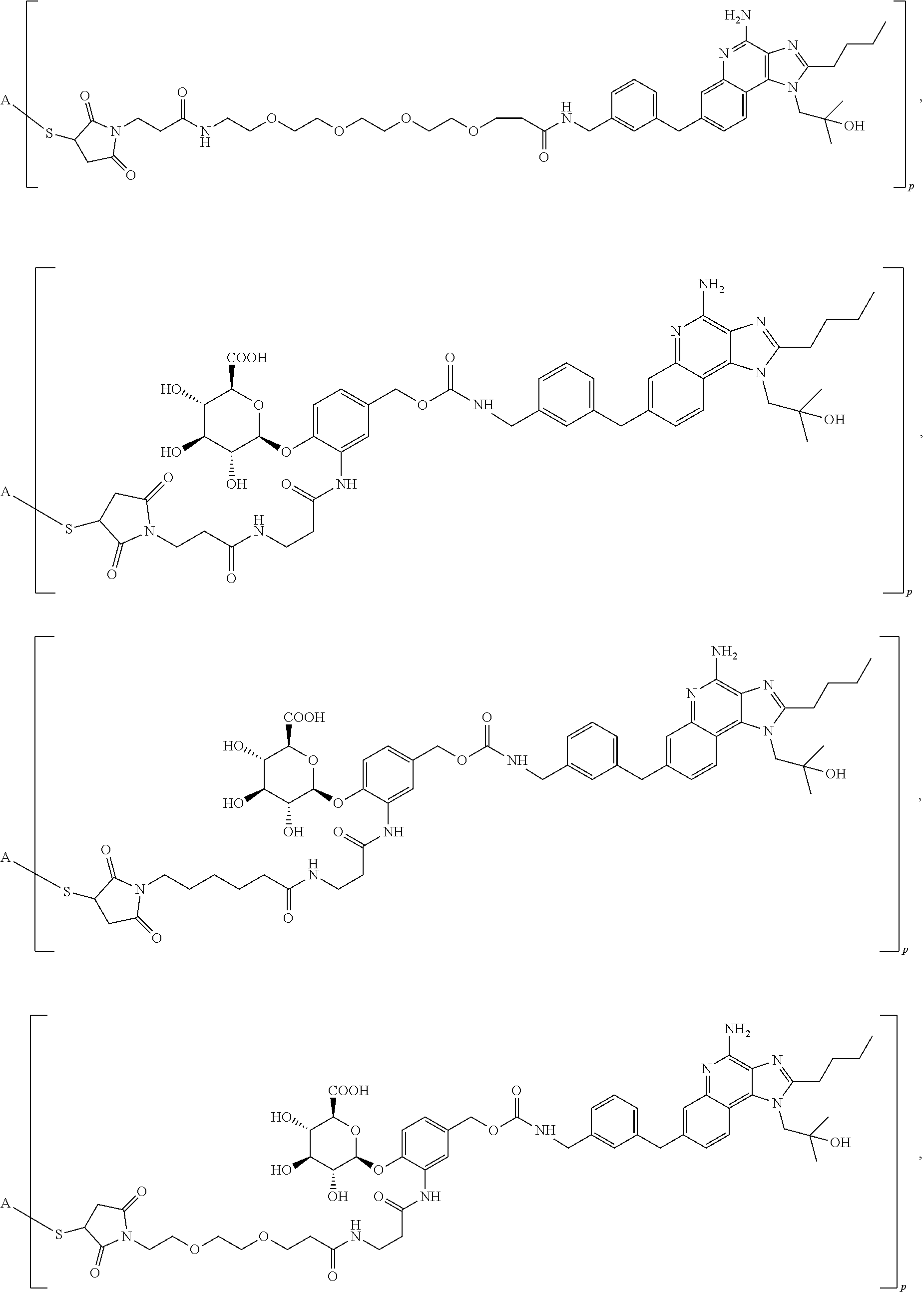

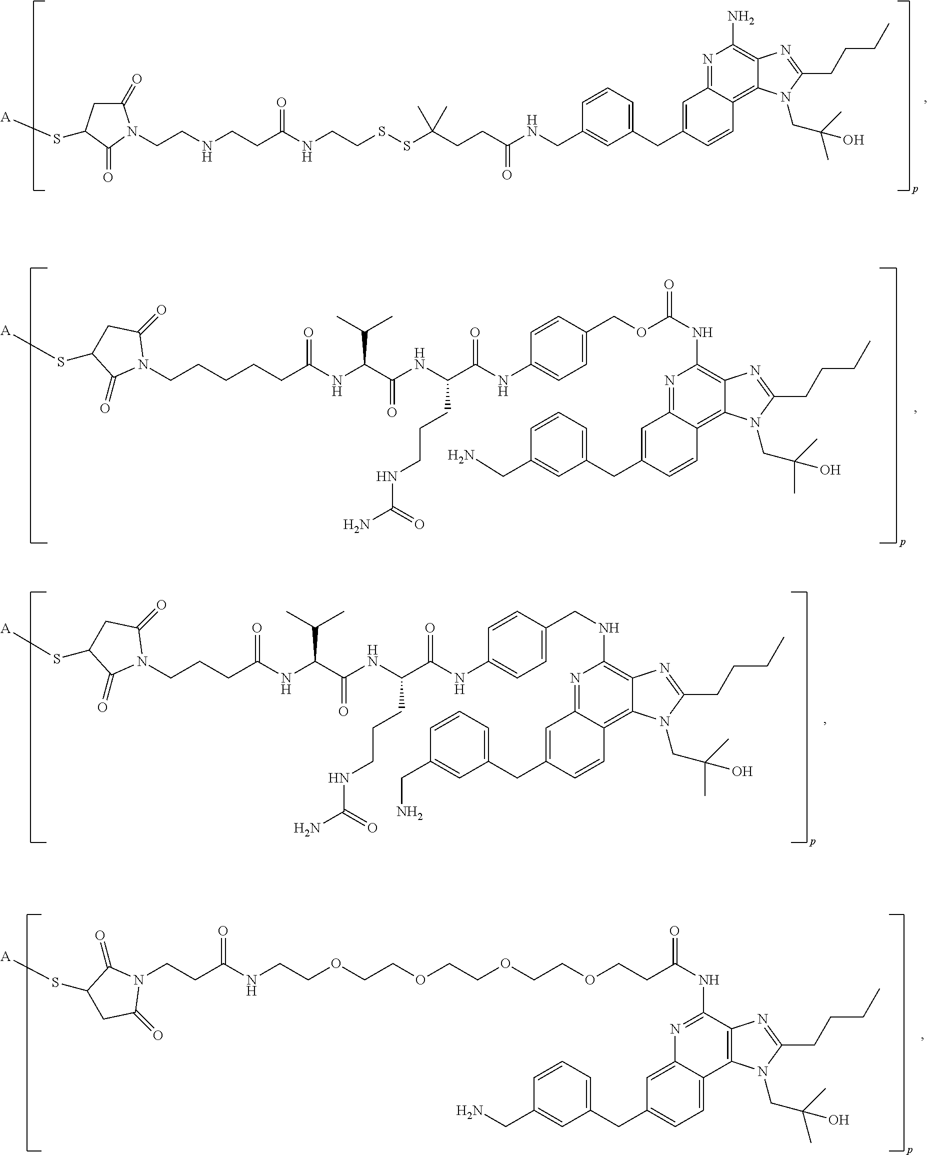

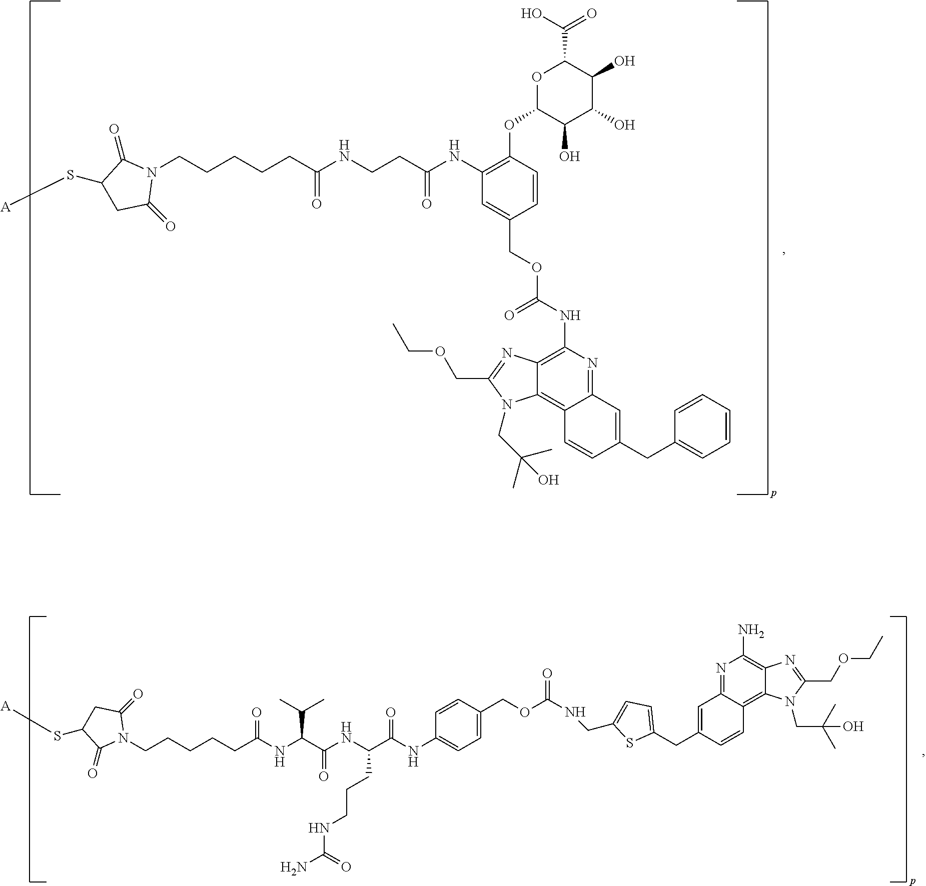

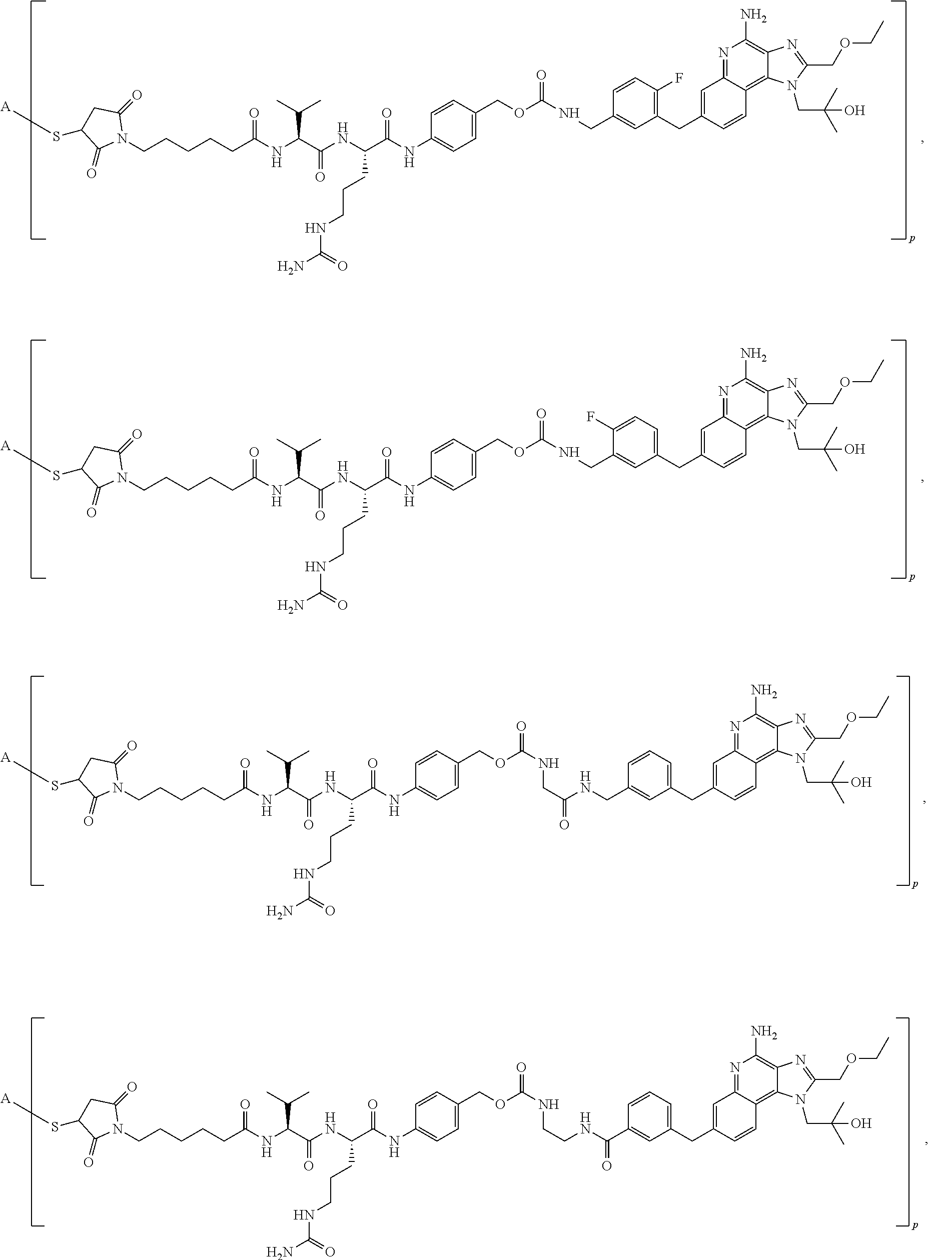

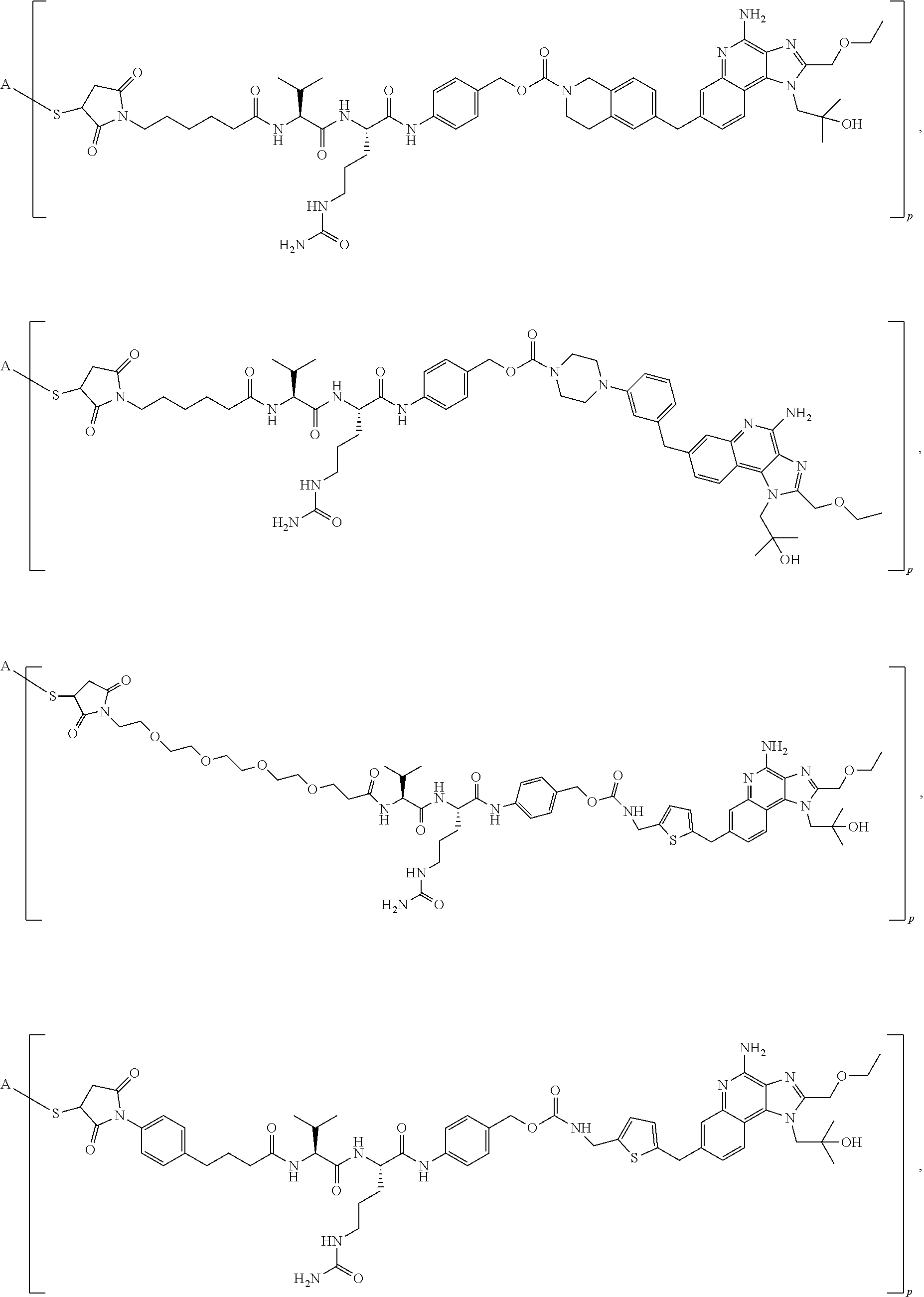

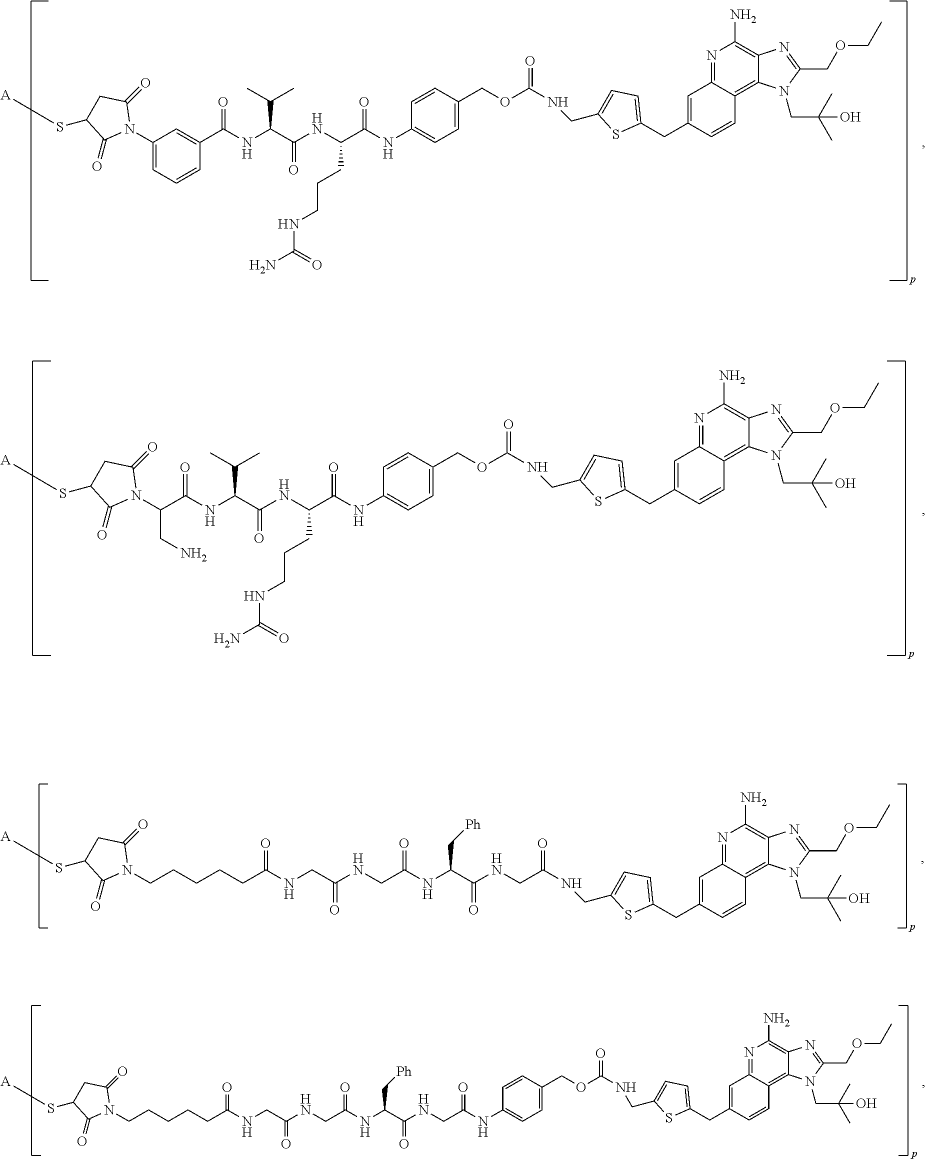

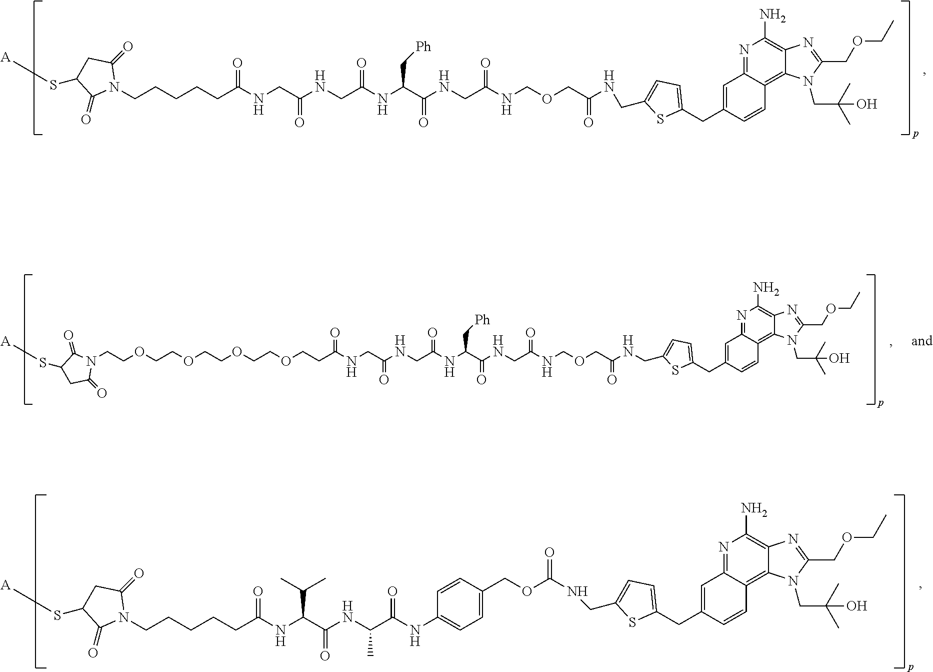

or a pharmaceutically acceptable salt thereof.

In some embodiments, the targeting moiety comprises an immunoglobulin, a protein, a peptide, a small molecule, a nanoparticle, or a nucleic acid.

In some embodiments, the targeting moiety comprises an antibody or antigen binding fragment thereof.

In certain embodiments, the antibody binds to one or more tumor-associated antigens or cell-surface receptors selected from BMPR1B, 1B7-H4, E16, STEAP1, MUC16, MPF, Napi2b, Sema 5b, PSCA hlg, ETBR, MSG783, STEAP2, TrpM4, CRIPTO, CD21, CD79b, FcRH2, HER2, NCA, MDP, IL20Ra, Brevican, EphB2R, ASLG659, PSCA, GEDA, BAFF-R, CD22, CD79a, CXCR5, HLA-DOB, P2X5, CD72, LY64, FcRH1, FcRH5, TEN1B2, PMEL 17, TMEFF1, GDNF-Ral, Ly6E, TMEM46, Ly6G6D, LGR5, RET, LY6K, GPR19, GPR54, ASPHD1, Tyrosinase, TMEM118, GPR172A, CD33, and CLL-1.

In certain embodiments, the antibody binds to HER2 or B7-H4.

In certain embodiments, the antibody binds to HER2.

In certain embodiments, the antibody is trastuzumab.

In another embodiment, the antigen binding fragment is a Fab, Fab′, F(ab′)2, single domain antibody, T and Abs dimer, Fv, scFv, dsFv, ds-scFv, Fd, linear antibody, minibody, diabody, bispecific antibody fragment, bibody, tribody, sc-diabody, kappa (lamda) body, BiTE, DVD-Ig, SIP, SMIP, DART, polymer or aptamer.

In some embodiments, the targeting moiety comprises a cell-interacting molecule.

In some embodiments, the targeting moiety comprises a ligand.

In some embodiments, the targeting moiety is capable of binding to a tumor antigen.

In certain embodiments, the targeting moiety binds to a molecule selected from the group consisting of: CD2, CD19, CD20, CD22, CD27, CD28, CD33, CD37, CD38, CD40, CD40L, CD44, CD47, CD52, CD56, CD70, CD79, CD86/80, CD113, CD122, CD137, CD155, CD160, CD206, 4-1BB, 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbB1, ErbB2, ErbB3, ErbB4, EGFL7, EpCAM, EphA2, EphA3, EphB2, FAP, Fibronectin, Folate Receptor, ASGPR, Ganglioside GM3, GD2, gp100, gpA33, GPNMB, ICOS, IGF1R, Integrin av, Integrin ανβ, KIR, LAG-3, Lewis Y, Mesothelin, c-MET, RON, PRLR, MN Carbonic anhydrase IX, MUC1, MUC16, Nectin-4, NKGD2, NOTCH, OX40, OX40L, PD-1, PDL1, PSCA, PSMA, RANKL, ROR1, ROR2, SLC44A4, Syndecan-1, TACI, TAG-72, Tenascin, TIM3, TREM-1, TREM-2, MACRO, Ly6E, TRAILR1, TRAILR2, VEGFR-1, VEGFR-2, VEGFR-3, FOLR1, TRPV6, FOLH1 (PMSA), GNRHR, Trop2, NECTIN4, LRP1, GLUT1, EGFR1, AXL, CA9, Claudin18.2, CLDN6, APN, DLL3, DLL4, CEACAM5, FZD10, TFRC , MET, SSTR2, CCKBR, LFA1, ICAM, GPR87, GM-CSF, GM-CSFR, CSF-1R, TLR family, GITRL, GITR, 4-BBL, , ICOSL, MHCII antigen, TCR, FLT3, c-KIT, CTLA-4, IGIT, Galectin-9, HVEM, VISTA, B7-H4, , phosphatidylserine, HHLA2, , Galectin-3, LILRB2, LILRB3, LILRB4, SIGLEC15, CLEC5a, TIGIT, TfR, NKG2A, NKG2D, SLAMF7, KIR2DL1, KIR2DL2, KIR2DL3, FGFR1, FGFR2, FGFR4, NeuGcGM3, CXCR4 and variants thereof.

### Synthesis of Conjugate Compounds

In order to form the conjugate compounds of the present disclosure, the linker can first reacts with the payload compound to provide the linker-payload compound with one reactive group, which can then react with the targeting moiety. Alternatively, one end of the linker can first react with the targeting moiety to provide a targeting moiety bearing a linking moiety with one reactive group bonded thereto, which can then react with the payload compound.

In some embodiments, the targeting moiety is an antibody. In some embodiments, the number of payload compounds bound per antibody molecule can be determined spectrophotometrically. In some embodiments, the average number of linked payload compounds per antibody molecule (DAR) is 2-12. In some embodiments, the DAR value is 2-10. In some embodiments, the DAR value is 2-8. In some embodiments, the DAR value is 2.5-4.0. In some embodiments, the DAR value is 4-8. In some embodiments, the DAR value is 5-8. In some embodiments, the DAR value is 6-8. In some embodiments, the DAR value is 6.5-8. In some embodiments, the DAR value is 7-8. In some embodiments, the DAR value is 7.1-8. In some embodiments, the DAR value is 7.2-8. In some embodiments, the DAR value is 7.3-8. In some embodiments, the DAR value is 7.4-8. In some embodiments, the DAR value is 7.5-8. In some embodiments, the DAR value is 7.6-8. In some embodiments, the DAR value is 7.7-8. In some embodiments, the DAR value is 7.8-8. In some embodiments, the DAR value is 7.9-8.

### Pharmaceutical Composition

For the purposes of administration, in some embodiments, the compounds provided herein are administered as a raw chemical or are formulated as pharmaceutical compositions.

Therefore, in a further aspect, the present disclosure provides pharmaceutical compositions comprising one or more compounds or a pharmaceutically acceptable salts thereof provided herein. In some embodiments, the pharmaceutical compositions of the present disclosure comprise a compound selected from Formula (I), Formula (Ia), Formula (II′), Formula (IIa′) or a pharmaceutically acceptable salt thereof. In some embodiments, the pharmaceutical compositions of the present disclosure comprise a first compound selected from Formula (I), Formula (Ia), Formula (II′), Formula (IIa′) or a pharmaceutically acceptable salt thereof and one or more additional compounds of the same formula but said first compound and additional compounds are not the same molecules.

As used herein, the term “pharmaceutical composition” is a formulation containing the compounds of the present disclosure in a form suitable for administration to a subject. The pharmaceutical compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In some embodiments, the pharmaceutical compositions of the present disclosure comprises a therapeutically effective amount of one or more compounds of the present disclosure or a pharmaceutically acceptable salt thereof.

As used herein, the term “therapeutically effective amount” refers to an amount of a molecule, compound, or composition comprising the molecule or compound to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; the rate of administration; the therapeutic or combination of therapeutics selected for administration; and the discretion of the prescribing physician. Therapeutically effective amounts for a given situation can be determined by routine experimentation that is within the skill and judgment of the clinician.

In some embodiments, the pharmaceutical composition comprises one or more compounds of the present disclosure, or a pharmaceutically acceptable salt thereof, and at least one pharmaceutical acceptable carrier or excipient.

As used herein, the term “pharmaceutically acceptable carrier” refers to a carrier or excipient that is useful in preparing a pharmaceutical composition that is generally safe, non-toxic and neither biologically nor otherwise undesirable, and includes carrier that is acceptable for veterinary use as well as human pharmaceutical use. A “pharmaceutically acceptable carrier” as used herein includes both one and more than one such carrier. The term “pharmaceutically acceptable carrier” also encompasses “pharmaceutically acceptable excipient” and “pharmaceutically acceptable diluent”. The particular carrier used in the pharmaceutical compositions of the present disclosure will depend upon the means and purpose for which the compounds of the present disclosure is being applied.

The pharmaceutical acceptable carrier employed can be, for example, a solid, liquid or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or non-aqueous techniques.

A tablet containing the pharmaceutical composition of the present disclosure may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient. For example, a formulation intended for the oral administration to humans may contain from about 0.5 mg to about 5 g of active agent, compounded with an appropriate and convenient amount of carrier material which may vary from about 0.05 to about 95 percent of the total composition.

Pharmaceutical compositions of the present disclosure suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present disclosure can be in a form suitable for topical use such as, for example, an aerosol, cream, ointment, lotion, dusting powder or the like. Further, the compositions can be in a form suitable for use in transdermal devices. These formulations may be prepared, utilizing a compound represented by Formula I of this invention or a pharmaceutically acceptable salt thereof, via conventional processing methods. As an example, a cream or ointment is prepared by admixing hydrophilic material and water, together with about 0.05 wt % to about 10 wt % of the compound, to produce a cream or ointment having a desired consistency.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical composition described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound provided herein or pharmaceutically acceptable salts thereof, may also be prepared in powder or liquid concentrate form.

In some embodiments, the pharmaceutical compositions of the present disclosure can be formulated as a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical carrier. The amount of the compounds provided herein in the unit dosage form will vary depending on the condition to be treated, the subject to be treated (e.g., the age, weight, and response of the individual subject), the particular route of administration, the actual compound administered and its relative activity, and the severity of the subject's symptoms.

In some embodiments, for oral administration, each dosage unit contains from about 0.01 mg to about 2000 mg of one or more compounds provided herein, for example, from about 0.01 mg to about 1000 mg, from about 0.02 mg to about 1000 mg, from about 1 mg to about 1000 mg, from about 2 mg to about 1000 mg, from about 3 mg to about 1000 mg, from about 4 mg to about 1000 mg, from about 5 mg to about 1000 mg, from about 10 mg to about 1000 mg, from about 25 mg to about 1000 mg, from about 50 mg to about 1000 mg, from about 100 mg to about 1000 mg, from about 200 mg to 1000 mg, from about 300 mg to about 1000 mg, from about 400 mg to about 1000 mg, from about 500 mg to about 1000 mg, from about 1 mg to 500 mg, from about 10 mg to about 500 mg, from about 50 mg to about 500 mg, from about 100 mg to about 500 mg, from about 200 mg to about 500 mg, from about 300 mg to about 500 mg, from about 400 mg to about 500 mg, for example about 1 mg, about 2 mg, about 3 mg, about 4 mg, about 5 mg, about 6 mg, about 7 mg, about 8 mg, about 9 mg, about 10 mg, about 25 mg, about 50 mg, about 75 mg, about 100 mg, about 150 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 600 mg, about 700 mg, about 800 mg, about 900 mg or about 1000 mg.

In some embodiments, for parenteral administration, each dosage unit contains from about 0.1 mg to about 100 mg of one or more compounds provided herein, for example from about 0.5 mg to about 100 mg, from about 1 mg to about 100 mg, from about 5 mg to about 100 mg, from about 10 mg to about 100 mg, from about 20 mg to about 100 mg, from about 30 mg to about 100 mg, from about 40 mg to about 100 mg, from about 50 mg to about 100 mg, from about 0.5 mg to about 90 mg, from about 0.5 mg to about 80 mg, from about 0.5 mg to about 70 mg, from about 0.5 mg to about 60 mg, from about 0.5 mg to about 50 mg, from about 0.5 mg to about 40 mg, from about 1 mg to about 90 mg, from about 5 mg to about 90 mg, from about 10 mg to about 80 mg, from about 20 mg to about 70 mg, from about 30 mg to about 60 mg, or from about 40 mg to about 50 mg.

In some embodiments, dosage levels of the pharmaceutical compositions of the present disclosure can be between 0.001-1000 mg/kg body weight/day, for example, 0.01-900 mg/kg body weight/day, 0.01-800 mg/kg body weight/day, 0.01-700 mg/kg body weight/day, 0.01-600 mg/kg body weight/day, 0.01-500 mg/kg body weight/day, 0.01-400 mg/kg body weight/day, 0.01-300 mg/kg body weight/day, 0.05-900 mg/kg body weight/day, 0.05-800 mg/kg body weight/day, 0.05-700 mg/kg body weight/day, 0.05-600 mg/kg body weight/day, 0.05-500 mg/kg body weight/day, 0.1-200 mg/kg body weight/day, 0.1-150 mg/kg body weight/day, 0.1-100 mg/kg body weight/day, 0.5-100 mg/kg body weight/day, 0.5-80 mg/kg body weight/day, 0.5-60 mg/kg body weight/day, 0.5-50 mg/kg body weight/day, or 1-50 mg/kg body weight/day. In some instances, dosage levels below the lower limit of the aforesaid range may be more than adequate, while in other cases still larger doses may be employed without causing any harmful side effect, provided that such larger doses are first divided into several small doses for administration throughout the day. For further information on routes of administration and dosage regimes, see Chapter 25.3 in Volume 5 of Comprehensive Medicinal Chemistry (Corwin Hansch; Chairman of Editorial Board), Pergamon Press 1990, which is specifically incorporated herein by reference.

In some embodiments, the pharmaceutical composition of the present disclosure comprising one or more compounds provided herein or pharmaceutically acceptable salts thereof further comprises one or more additional therapeutically active agents. Examples of the additional therapeutically active agents include but not limited to an anti-viral agent, a chemotherapeutic agent, radiation, an anti-tumor vaccine, an antiviral vaccine, cytokine therapy, a tyrosine kinase inhibitor, or an immuno-oncology agent. The immuno-oncology agents include but not limited to small molecule drug, antibody, or other biologic molecule. Examples of biologic immuno-oncology agents include, but are not limited to, cancer vaccines, antibodies, and cytokines. In some embodiment, the immuno-oncology agents could antagonist of a protein that inhibits T cell and/or NK cell activation. In some embodiment, the antibody is a monoclonal antibody.

### Method of Treatment of Disease

In an aspect, there is provided a method for the prophylaxis or treatment of a disease mediated by toll-like receptors 7 and/or 8 in a subject in need thereof, comprising administering a therapeutically effective amount of the compound(s) or a pharmaceutically acceptable salts thereof.

As used herein, the term “treatment” or “treating” describes the management and care of a patient for the purpose of reversing, inhibiting, or combating a disease, condition, or disorder. As used herein, the term “prophylaxis” refers to a measure taken to maintain health and prevent the spread of a disease, condition or disorder.

In some embodiments, the disease mediated by toll-like receptors 7 and/or 8 is cancer.

As used herein, the term “cancer” and “cancerous” refer to or describe the physiological condition in mammals that is typically characterized by unregulated cell growth. Examples of cancer include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric cancer including gastrointestinal cancer, pancreatic cancer, glioblastoma, neuroblastoma, cervical cancer, ovarian cancer, liver cancer, bladder cancer, urinary tract cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophageal cancer, tumors of the biliary tract, as well as head and neck cancer.

In certain embodiments, the cancer is selected from breast cancer, bladder cancer, head and neck cancer, non-small cell lung cancer, small cell lung cancer, colorectal cancer, gastrointestinal stromal, gastroesophageal carcinoma, renal cell cancer, prostate cancer, liver cancer, colon cancer, pancreatic cancer, ovarian cancer, lymphoma, cutaneous T-cell lymphoma, visceral tumors or melanoma.

In some embodiments, the disease mediated by toll-like receptors 7 and/or 8 is viral infection.

As used herein, the term “viral infections” include, but are not limited to, diseases caused by RNA virus, DNA virus, hepatitis A, hepatitis B (HBV), hepatitis C (HCV), hepatitis D (HDV), human immunodeficiency virus (HIV), human papillomavirus (HPV), respiratory syncytial virus (RSV), severe acute respiratory syndrome (SARS), influenza, parainfluenza, cytomegalovirus, dengue, herpes simplex virus 1, herpes simplex virus 2, Coxsackie (CV), coronavirus, Epstein-Barr virus (EBV), encephalomyocarditis (EMCV), influenza A (IAV), measles (MV), Sendai (SV), vesicular stomatitis (VSV) virus, leishmania infection and respiratory syncytial virus. In certain embodiments, the viral infections include diseases caused by such as hepatitis A, hepatitis B (HBV), hepatitis D (HDV), HIV, human papillomavirus (HPV), respiratory syncytial virus (RSV), severe acute respiratory syndrome (SARS), influenza, parainfluenza, cytomegalovirus, dengue, herpes simplex virus 1, herpes simplex virus 2, Coxsackie (CV), encephalomyocarditis (EMCV), influenza A (IAV), measles (MV), Sendai (SV), vesicular stomatitis (VSV) virus, leishmania infection and respiratory syncytial virus.

In certain embodiments, the viral infection is from a virus selected from the group consisting of hepatitis B virus (HBV), hepatitis C virus (HCV), human immunodeficiency virus (HIV), human papillomavirus (HPV), Coxsackie (CV), coronavirus, Epstein-Barr virus (EBV), encephalomyocarditis (EMCV), influenza A (IAV), measles (MV), Sendai (SV), or vesicular stomatitis (VSV) virus.

In some embodiments, the subject may have not previously received antiviral treatment (treatment naive). In some embodiments, the subject may have previously received antiviral treatment (treatment experienced). In some embodiments, the subject may have previously received antiviral treatment and developed resistance to the previously received antiviral treatment.

In some embodiments, the compounds, or a pharmaceutically acceptable salts thereof or the pharmaceutical composition of the present disclosure can be administered as the sole active agent. In some embodiments, the compounds, or the pharmaceutically acceptable salts thereof or the pharmaceutical composition of the present disclosure can be administered in combination with one or more additional active ingredients. The skilled artisan will recognize that a variety of active ingredients may be combined with the compounds of the present disclosure. In some embodiments, the additional active ingredient of the pharmaceutical combination formulation or dosing regimen has complementary activities to the compounds of disclosure such that they do not adversely affect each other. Such ingredients are suitably present in combination in amounts that are effective for the purpose intended.

In some such embodiment, the compounds or a pharmaceutically acceptable salts thereof provided herein, or the pharmaceutical composition(s) provided herein, can be used in combination with an additional therapy. The additional therapy may be optionally include one or more therapeutic agents, radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing.

In some embodiments, the compounds or a pharmaceutically acceptable salts thereof provided herein, or the pharmaceutical composition(s) provided herein may be administered simultaneously, sequentially or separately with one or more additional therapeutic agents.

In some embodiments, the compound(s) or a pharmaceutically acceptable salts thereof of the present disclosure, or the pharmaceutical composition(s) provided herein can be administered to the subject a therapeutically effective amount of an anti-viral agent, a chemotherapeutic agent, radiation, an anti-tumor vaccine, an antiviral vaccine, cytokine therapy and a tyrosine kinase inhibitor prior to, simultaneously with or after administration of the compound(s).

In further aspect, there is provided use of the compound or a pharmaceutically acceptable salts thereof provided herein, or the pharmaceutical composition(s) provided herein in the manufacture for treating viral infection or cancer.

In another aspect, there is provided a method for activating toll-like receptors 7 and/or 8 in a subject in need thereof, comprising administering the compound(s) or a pharmaceutically acceptable salts thereof provided herein, or the pharmaceutical composition(s) provided herein.

The term “agonist” refers to a substance that stimulates its binding partner, typically a receptor. Stimulation is defined in the context of the particular assay, or may be apparent in the literature from a discussion herein that makes a comparison to a factor or substance that is accepted as an “agonist” of the particular binding partner under substantially similar circumstances as appreciated by those of skill in the art. Stimulation may be defined with respect to an increase in a particular effect or function that is induced by interaction of the agonist or partial agonist with a binding partner and can include allosteric effects. “Activating toll-like receptors 7 and/or 8” refers to an increase in TLR 7 and/or 8 activity as compared to the activity of that enzyme in the absence of the compounds of the present disclosure. In some embodiments, such increase in TLR 7 and/or 8 and/or TLR 7 and/or 8 variants activity can be a direct or indirect response to the presence of a compound provided herein relative to the TLR 7 and/or 8 activity in the absence of the compound provided herein. In some embodiments, the stimulation of TLR 7 and/or 8 activity may be compared in the same subject prior to treatment, or other subjects not receiving the treatment.

In some embodiments, the compound(s) or a pharmaceutically acceptable salts thereof provided herein, or the pharmaceutical composition(s) provided herein can be utilized to inhibit, block, reduce or decrease TLR 7 and/or 8 activation for the reduction of tumor growth and the modulation of dysregulated immune responses e.g. to block immunosuppression and increase immune cell activation and infiltration in the context of cancer and cancer immunotherapy.

In a further aspect, there is provided a method for stimulating an immune response in a subject in need thereof, comprising administering the compound(s) or a pharmaceutically acceptable salts thereof provided herein, or the pharmaceutical composition(s) provided herein.

### EXAMPLES

For the purpose of illustration, the following examples are included. The Examples provided herein describe the synthesis of compounds disclosed herein as well as intermediates used to prepare the compounds. However, it is to be understood that these examples do not limit the present disclosure and are only meant to suggest a method of practicing the present disclosure. Persons skilled in the art will recognize that the chemical reactions described may be readily adapted to prepare a number of other compounds of the present disclosure, and alternative methods for preparing the compounds of the present disclosure are deemed to be within the scope of the present disclosure. For example, the synthesis of non-exemplified compounds according to the present disclosure may be successfully performed by modifications apparent to those skilled in the art, e.g., by appropriately protecting interfering groups, by utilizing other suitable reagents and building blocks known in the art other than those described, and/or by making routine modifications of reaction conditions. Besides, persons skilled in the art will also understand that individual steps described herein or in the separate batches of a compound may be combined. Alternatively, other reactions disclosed herein or known in the art will be recognized as having applicability for preparing other compounds of the present disclosure. The following description is, therefore, not intended to limit the scope of the present disclosure, but rather is specified by the claims appended hereto.

The following abbreviations have been used in the examples:

| | |
|---|---|
| AcOH | Acetic acid |
| aq. | aqueous |
| Boc₂O | Di-tert-butyl dicarbonate |
| CbzCl | Benzyl chloroformate |
| CDI | Carbonyldiimidazole |
| DCM | Dichloromethane |
| DIEA | N,N-Diisopropylethylamine |
| DMAP | 4-Dimethylaminopyridine |
| DMF | Dimethylformamide |
| DMSO | Dimethyl sulfoxide |
| EA | Ethyl acetate |
| EDCI | 1-Ethyl-3-(3-dimethylaminopropyl)carbodiimide |
| equiv. | equivalent |
| EtOH | ethanol |
| EtOAc | Ethyl acetate |
| Et₃N | Triethylamine |
| g | gram |
| h or hr | hour(s) |
| HOBT | Hydroxybenzotriazole |
| H₂O | water |
| HPLC | high pressure liquid chromatography |
| KOAc | Potassium acetate |
| LC-MS or | Liquid Chromatography-Mass Spectroscopy |
| LCMS | |
| M | Mole per litter |
| MeCN | Acetonitrile |
| MeOH | Methanol |
| min | minute(s) |
| mL | milliliter(s) |
| mmol | millimolar |
| Ms₂O | Methanesulfonic anhydride |
| Pd(PPh₃)Cl₂ | Bis(triphenylphosphine)palladium chloride |
| PE | Polyethylene |
| POCl₃ | Phosphoryl chloride |
| rt or r.t. | room temperature |
| SFC | Supercritical fluid chromatography |
| TEA | Triethylamine |
| TGI | Tumor Growth Inhibition |
| THF | Tetrahydrofuran |
| TM | Target molecule |
| TrtCl | Triphenylmethyl chloride |

### Example 1

### Preparation of 7-bromo-2,4-dichloro-3-nitroquinoline (Int-6)

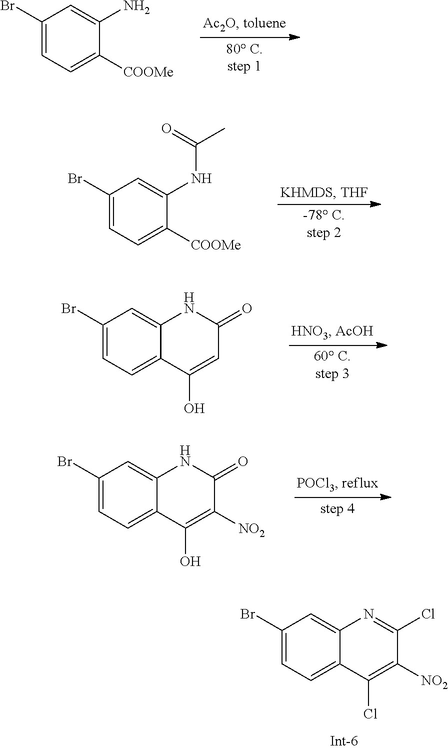 [SMILES: COC(=O)c1ccc(Br)cc1N ; COC(=O)c1ccc(Br)cc1NC(C)=O ; O=c1cc(O)c2ccc(Br)cc2[nH]1 ; O=c1[nH]c2cc(Br)ccc2c(O)c1[N+](=O)[O-] ; O=[N+]([O-])c1c(Cl)nc2cc(Br)ccc2c1Cl]

### Step 1: Preparation of methyl 2-acetamido-4-bromobenzoate

To a suspension of methyl 2-amino-4-bromobenzoate (600 g, 2.6 mol) in toluene (6000 mL) was added acetic anhydride (399.2 g, 3.9 mol) at room temperature. The mixture was heated at 80° C. for 16 hours. LCMS showed the reaction was completed. The reaction mixture was concentrated under reduced pressure and the residual solid was triturated with PE/EtOAc (4400 mL, 10/1) and dried under vacuum to give methyl 2-acetamido-4-bromobenzoate (600 g, 84.8% yield) as a white solid.

LCMS (ESI) calcd for C₁₀H₁₀BrNO₃ [M+H]⁺ m/z=273.1, found 273.0.

### Step 2: Preparation of 7-bromo-4-hydroxyquinolin-2(1H)-one

To a solution of potassium bis(trimethylsilyl)amide in THF (1M, 6.6 L, 6.6 mol) were added suspensions of methyl 2-acetamido-4-bromobenzoate (600 g, 2.2 mol) in extra dry THF (6 L) slowly at −78° C. under nitrogen atmosphere. After stirring at this temperature for 1 hours the mixture was allowed to warm 10° C. within 1 hours. LCMS showed the reaction was completed. The reaction mixture was quenched with water (36 L). The aqueous layer was washed with EtOAc (12 L *2). The separated aqueous layer was cooled to 10° C. and the pH was adjusted to 2.5-3.5 with the addition of 5N HCl aqueous solution resulting in a yellow precipitate. The solid was collected by filtration, washed by EtOAc (12 L) and dried under vacuum to give 7-bromo-4-hydroxyquinolin-2(1H)-one (400 g, 75.8%) as an off-white solid.

LCMS (ESI) calcd for C₉H₆BrNO₂ [M+H]⁺ m/z=240.0, found 240.0.

### Step 3: 7-bromo-4-hydroxy-3-nitroquinolin-2(1H)-one

A suspension of 7-bromoquinoline-2,4-diol (400 g, 1.67 mol) and 70% nitric acid (225 mL, 2.5 mol) in acetic acid (4000 mL) was heated at 60° C. for 2 hours. LCMS showed the reaction was completed. The reaction mixture was cooled to room temperature. The solid was collected by filtration, washed with ice water (1000 mL) and dried under vacuum to give 7-bromo-4-hydroxy-3-nitroquinolin-2(1H)-one (350 g, 73.5%) as a yellow solid.

LCMS (ESI) calcd for C₉H₅BrN₂O₄[M+H]⁺ m/z=284.9, found 285.0.

### Step 4: 7-bromo-2,4-dichloro-3-nitroquinoline (Int 6)

To solution of 7-bromo-4-hydroxy-3-nitroquinolin-2(1H)-one (350 g, 1.23 mol) in POCl₃ (2800 mL) was added TEA (149 g, 1.48 mol) at 0° C. The mixture heated at reflux for 3 hours. After cooling to ambient temperature, the mixture was concentrated in vacuo. The residue was poured into ice-water and extracted with DCM (1000 mL*3). The combined organic phases were washed with brine, dried over Na₂SO₄, concentrated and purified by silica gel column chromatography (eluting with EtOAc/PE, 0% to 1%) to give 7-bromo-2,4-dichloro-3-nitroquinoline (300 g, 75.7% yield) as a yellow solid.

¹H NMR (400 MHz, DMSO-d₆, ppm) δ 8.47 (d, J=1.9 Hz, 1H), 8.27 (d, J=9.0 Hz, 1H), 8.13 (dd, J=9.0, 1.9 Hz, 1H).

### Example 2

### Preparation of 7-bromo-2-(ethoxymethyl)-1H-imidazo[4,5-c]quinolin-4-amine (11A)

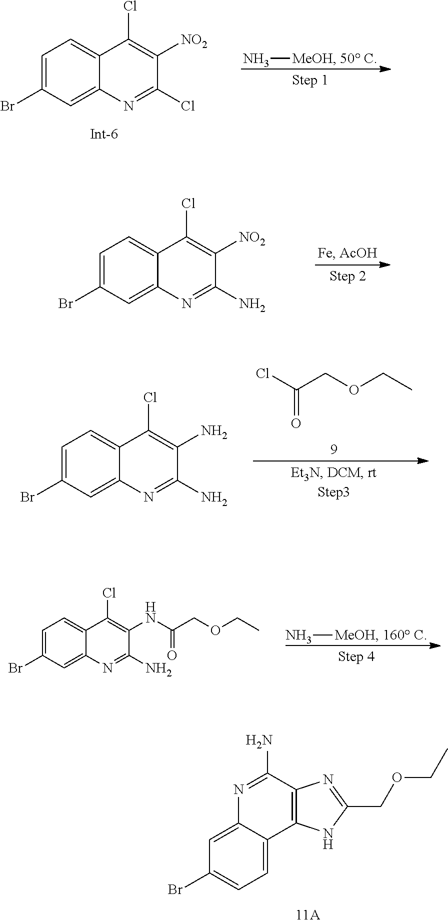

### Step 1: 7-bromo-4-chloro-3-nitroquinolin-2-amine

A solution of 7-bromo-2,4-dichloro-3-nitroquinoline (50 g, 156.25 mmol) in NH3-MeOH (100 mL) was stirred at 160° C. for 8 hours in a high-pressure reactor. After cooling to ambient temperature, the mixture was concentrated in vacuo. The residue was purified by silica gel column chromatography (PE:EA 2:1) to afford the title compound (35 g, 74.1% yield) as a yellow solid.

LCMS (ESI) calcd for C₉H₅BrN₃O₂[M+H]⁺ m/z=301.9, found 302.

### Step 2: 7-bromo-4-chloroquinoline-2,3-diamine

To a solution of 7-bromo-4-chloro-3-nitroquinolin-2-amine (35 g, 115.89 mmol) in AcOH (200 mL) was added Fe (64.9 g, 1.158 mol) at 0° C. The reaction mixture was stirred at RT for 10 hours. LCMS showed the reaction was completed. It was diluted with DCM and the solution was filtered through celite and the filtrate was concentrated to give the crude. The residue was purified by silica gel column chromatography (PE:EA 1:1) to give the title compound (25 g, 79.3% yield) as a yellow solid.

LCMS (ESI) calcd for C₉H₇BrClN₃ [M+H]⁺ m/z=271.9, found 272.

### Step 3: N-(2-amino-7-bromo-4-chloroquinolin-3-yl)-2-ethoxyacetamide

To a solution of 7-bromo-4-chloroquinoline-2,3-diamine (25 g, 91.73 mmol) and TEA (27.85 g, 275.20 mmol) in DCM (200 mL) was added 2-ethoxyacetyl chloride (16.86 g, 137.60 mmol) at 0° C. The reaction mixture was stirred at RT for overnight. LCMS showed the reaction was completed. The reaction was diluted with water and extracted with DCM (200 mL×3). The combine organic phases were washed with brine, dried over Na₂SO₄, concentrated to give the crude. The residue was purified by silica gel column chromatography (PE:EA 2:1) to give the title compound (28 g, 85.2% yield) as a yellow solid.

LCMS (ESI) calcd for C₁₃H₁₃BrClN₃O₂ [M+H]⁺ m/z=358.0, found 358.

### Step 4: 7-bromo-2-(ethoxymethyl)-1H-imidazo[4,5-c]quinolin-4-amine (11A)

A solution of N-(2-amino-7-bromo-4-chloroquinolin-3-yl)-2-ethoxyacetamide (28 g, 78.2 mmol) in NH₃-MeOH (60 mL) was stirred at 160° C. for 8 h in a high-pressure reactor. After cooling to ambient temperature, the mixture was concentrated in vacuo. The residue was purified by silica gel column chromatography (PE:EA 2:1) to afford the title compound (21 g, 83.6% yield) as a yellow solid.

LCMS (ESI) calcd for C₁₃H₁₃BrN₄O [M+H]⁺ m/z=321.0, found 321.

### Example 3

### Preparation of 7-bromo-2-(pentan-2-yl)-1H-imidazo[4,5-c]quinolin-4-amine (11B)

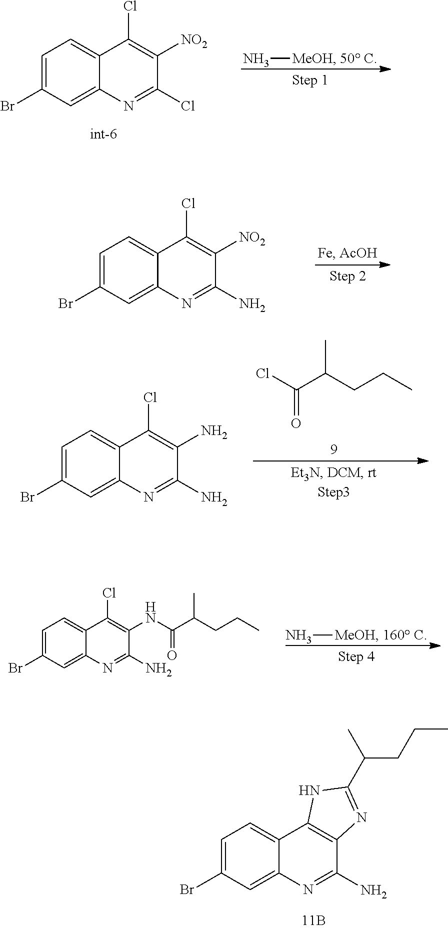

### Step 1: Preparation of 7-bromo-4-chloro-3-nitroquinolin-2-amine

A solution of 7-bromo-2,4-dichloro-3-nitroquinoline (20 g, 0.062 mol) in NH₃-MeOH (40 mL) was stirred at 50′C for 8 hours in a high-pressure reactor. LCMS showed the reaction was completed. The resulting mixture was cooled to RT and concentrated to give the crude title compound without purification.

LCMS (ESI) calcd for C₉H₅BrClN₃O₂[M+H]⁺ m/z=301.9, found 302.

### Step 2: Preparation of 7-bromo-4-chloroquinoline-2,3-diamine

To a solution of 7-bromo-4-chloro-3-nitroquinolin-2-amine (16 g, 0.053 mol) in AcOH (200 mL) was added Fe (29.6 g, 0.53 mol) at RT. The mixture was stirred at RT for 6 hours. LCMS showed the reaction was completed. It was diluted with DCM and the solution was filtered through celite. The filtrate was concentrated under vacuum to give the crude. The residue was diluted with water (100 mL) and extracted with EA (100 mL). The organic phase was washed with brine, dried over Na₂SO₄, concentrated to give the crude title compound. The residue was purified by combi-flash (PE:EA=1:5) to afford 7-bromo-4-chloroquinoline-2,3-diamine (10 g, 69.38% yield) as yellow solid.

LCMS (ESI) calcd for C₉H₇BrClN₃ [M+H]⁺ m/z=271.9, found 272.

### Step 3: Preparation of N-(2-amino-7-bromo-4-chloroquinolin-3-yl)-2-methylpentanamide

To a solution of 7-bromo-4-chloroquinoline-2,3-diamine (10 g, 36.7 mmol) and Et₃N (5.58 g, 55.1 mmol) in DCM (20 mL) was added 2-methylpentanoyl chloride (5.4 g, 40.4 mmol) dropwise at 0′C. Then the mixture was stirred at RT for 3 hours. LCMS showed the reaction was completed. The resulting mixture was diluted with water (50 mL) and extracted with DCM (50 mL×3). The organic phase was washed with brine, dried over Na₂SO₄, concentrated to give the crude title compound. The residue was purified by combi-flash (PE:EA=1:1) to afford N-(2-amino-7-bromo-4-chloroquinolin-3-yl)-2-methylpentanamide (10 g, 73.52% yield) as yellow solid.

LCMS (ESI) calcd for C₁₅H₁₇BrClN₃O [M+H]⁺ m/z=370.1, found 370.

### Step 4: Preparation of 7-bromo-2-(pentan-2-yl)-1H-imidazo[4,5-c]quinolin-4-amine (11B)

A solution of N-(2-amino-7-bromo-4-chloroquinolin-3-yl)-2-methylpentanamide (10 g, 0.027 mol) in NH₃-MeOH (30 mL) was stirred at 160° C. for 8 hours in a high-pressure reactor. After cooling to ambient temperature, the mixture was concentrated in vacuo. The residue was washed with PE:EA (1:1) to give 1-(4-amino-7-bromo-2-(ethoxymethyl)-1H-imidazo[4,5-c]quinolin-1-yl)-2-methylpropan-2-ol (8 g, 88.9% yield) as yellow solid.

LCMS (ESI) calcd for C₁₅H₁₇BrN₄ [M+H]⁺ m/z=333.1, found 333.

¹H NMR (400 MHz, DMSO-d₆) δ 7.75 (s, 1H), 7.43-7.29 (m, 5H), 3.19-3.07 (m, 1H), 1.91-1.79 (m, 1H), 1.72-1.60 (m, 1H), 1.39 (d, J=7.0 Hz, 3H), 1.33-1.17 (m, 2H), 0.88 (t, J=7.3 Hz, 3H).

### Example 4

### Preparation of di-tert-butyl (7-bromo-1-(2-((tert-butoxycarbonyl) oxy)-2-methylpropyl)-2-(ethoxymethyl)-1H-imidazo[4,5-c]quinolin-4-yl) iminodicarbonate (13A)

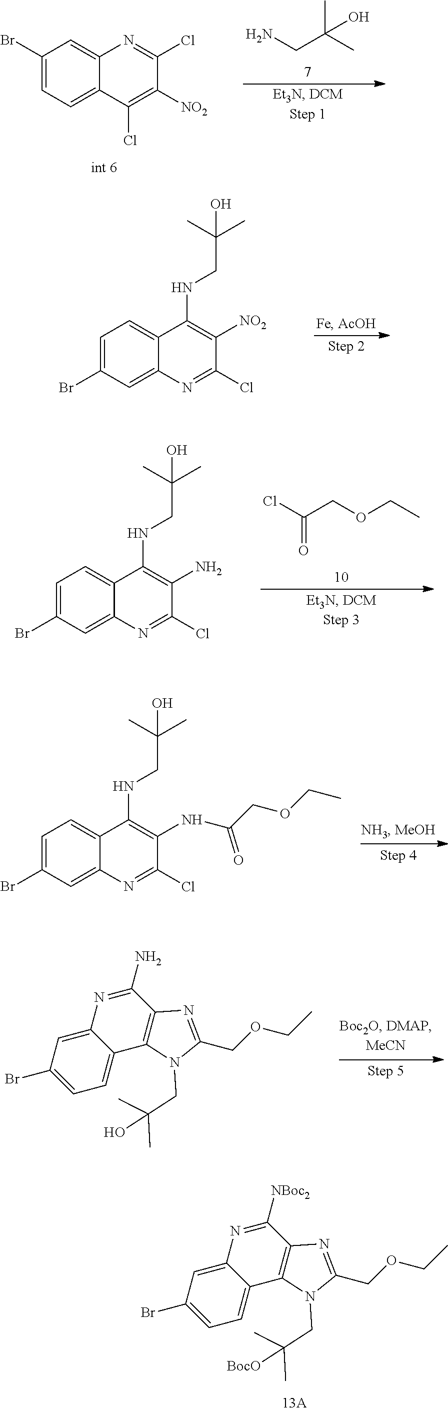

### Step 1: Preparation of 1-((7-bromo-2-chloro-3-nitroquinolin-4-yl) amino)-2-methylpropan-2-ol

To a solution of 7-bromo-2,4-dichloro-3-nitroquinoline (30 g, 93.2 mmol) and Et₃N (14.15 g, 139.8 mmol) in DCM (100 mL) was added 1-amino-2-methylpropan-2-ol (9.14 g, 102.5 mmol) dropwise at 0° C. The mixture was stirred at RT for 4 hours. LCMS showed the reaction was completed. The resulting mixture was diluted with water (60 mL) and extracted with DCM (60 mL×3). The combine organic phases were washed with brine, dried over Na₂SO₄, concentrated to give the crude.

LCMS (ESI) calcd for C₁₃H₁₃BrClN₃O₃ [M+H]⁺ m/z=374.4, found 374.

### Step 2: Preparation of 1-((3-amino-7-bromo-2-chloroquinolin-4-yl) amino)-2-methylpropan-2-ol

To a solution of 1-((7-bromo-2-chloro-3-nitroquinolin-4-yl) amino)-2-methylpropan-2-ol (32 g, 0.086 mol) in AcOH (260 mL) was added Fe (48 g, 0.86 mol) at RT. The mixture was stirred at RT for 6 hours. LCMS showed the reaction was completed. It was diluted with DCM and the solution was filtered through celite. The filtrate was concentrated under vacuum to give the crude. The residue was diluted with water (100 mL) and extracted with EA (100 mL×3). The combine organic phases were washed with brine, dried over Na₂SO₄, concentrated to give the crude. The residue was purified by silica gel column chromatography (PE:EA 3:1) to give the title compound (20 g, 67.96% yield) as yellow solid.

LCMS (ESI) calcd for C₁₃H₁₅BrClN₃O [M+H]⁺ m/z=344.3, found 344.

### Step 3: Preparation of N-(7-bromo-2-chloro-4-((2-hydroxy-2-methylpropyl) amino) quinolin-3-yl)-2-ethoxyacetamide

To a solution of 1-((3-amino-7-bromo-2-chloroquinolin-4-yl) amino)-2-methylpropan-2-ol (20 g, 58.0 mmol) and Et₃N (8.8 g, 86.9 mmol) in DCM (40 mL) was added 2-ethoxyacetyl chloride (7.8 g, 63.9 mmol) dropwise at 0° C. Then the mixture was stirred at RT for 3 hrs. LCMS showed the reaction was completed. The resulting mixture was diluted with water (60 mL) and extracted with DCM (60 mL×3). The combine organic phases were washed with brine, dried over Na₂SO₄, concentrated to give the crude. The residue was purified by silica gel column chromatography (PE:EA 1:3) to give the title compound (18 g, 71.96% yield) as yellow solid.

LCMS (ESI) calcd for C₁₇H₂₁BrClN₃O₃[M+H]⁺ m/z=430.1, found 430.

### Step 4: Preparation of 1-(4-amino-7-bromo-2-(ethoxymethyl)-1H-imidazo[4,5-c]quinolin-1-yl)-2-methylpropan-2-ol

A solution of N-(7-bromo-2-chloro-4-((2-hydroxy-2-methylpropyl) amino) quinolin-3-yl)-2-ethoxyacetamide (18 g, 0.042 mol) in NH₃-MeOH (50 mL) was stirred at 160° C. for 8 hours in a high-pressure reactor. After cooling to ambient temperature, the resulting mixture was concentrated in vacuo. The residue was washed with PE:EA (1:1) to give 1-(4-amino-7-bromo-2-(ethoxymethyl)-1H-imidazo[4,5-c]quinolin-1-yl)-2-methylpropan-2-ol (16 g, 97.28% yield) as yellow solid.

LCMS (ESI) calcd for C₁₇H₂₁BrN₄O₂[M+H]⁺ m/z=393.1, found 393.

### Step 5: Preparation of di-tert-butyl(7-bromo-1-(2-((tert-butoxycarbonyl)oxy)-2-methylpropyl)-2-(ethoxymethyl)-1H-imidazo[4,5-c]quinolin-4-yl) iminodicarbonate (13A)

To a solution of 1-(4-amino-7-bromo-2-(ethoxymethyl)-1H-imidazo[4,5-c]quinolin-1-yl)-2-methylpropan-2-ol (16 g, 0.041 mol) and DMAP (2 g, 0.016 mol) in MeCN (80 mL) was added Boc₂O (62.18 g, 0.28 mmol) at 75° C. The mixture was stirred at 75° C. for 4 hours. The resulting mixture was diluted with water (80 mL) and extracted with EA (100 mL×3). The combine organic phases were washed with brine, dried over Na₂SO₄, concentrated to give the crude. The residue was purified by silica gel column chromatography (PE:EA 2:1) to give the title compound (18 g, 63.79% yield) as yellow solid.

LCMS (ESI) calcd for C₃₂H₄₅BrN₄O₈ [M+H]⁺ m/z=693.2, found 693.

¹H NMR (400 MHz, DMSO) δ 8.60 (d, J=9.1 Hz, 1H), 8.25 (d, J=2.1 Hz, 1H), 7.78 (dd, J=9.1, 2.1 Hz, 1H), 3.57 (q, J=7.0 Hz, 2H), 1.99 (s, 2H), 1.56-1.35 (m, 5H), 1.28 (s, 18H), 1.21 (s, 9H), 1.19-1.12 (m, 6H).

### Example 5

### Preparation of di-tert-butyl (7-bromo-1-(2-((tert-butoxycarbonyl)oxy)-2-methylpropyl)-2-(pentan-2-yl)-1H-imidazo[4,5-c]quinolin-4-yl)iminodicarbonate (13B)

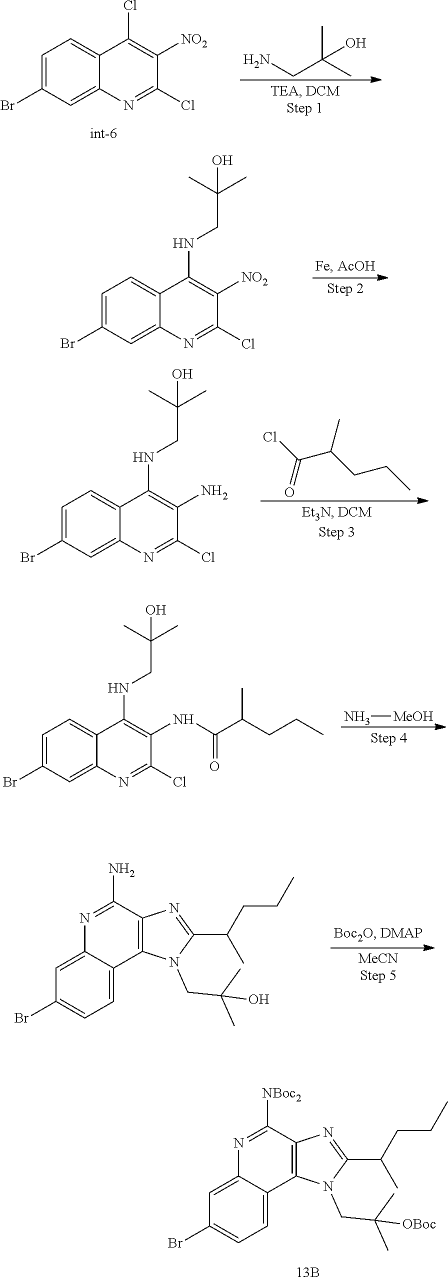

### Step 1: Preparation of 1-((7-bromo-2-chloro-3-nitroquinolin-4-yl)amino)-2-methylpropan-2-ol

To a solution of 7-bromo-2,4-dichloro-3-nitroquinoline (50 g, 155.31 mmol) in DCM (500 mL) was added TEA (47.15 g, 465.93 mmol) and 1-amino-2-methylpropan-2-ol (13.84 g, 155.31 mmol) at room temperature. The mixture was stirred at RT for overnight. LCMS showed the reaction was completed. The reaction was diluted with DCM and washed with brine and water, dried over Na₂SO₄, concentrated in vacuo to give the crude without purification.

LCMS (ESI) calcd for C₁₃H₁₃BrClN₃O₃[M+H]⁺ m/z=374.4, found 374.

### Step 2: Preparation of 1-((3-amino-7-bromo-2-chloroquinolin-4-yl)amino)-2-methylpropan-2-ol (int 8)

To a solution of 1-((7-bromo-2-chloro-3-nitroquinolin-4-yl)amino)-2-methylpropan-2-ol (50 g, 133 mmol) in AcOH (600 mL) was added Fe (74.8 g, 1.33 mol) at RT. The mixture was stirred at RT for 6 hours. LCMS showed the reaction was completed. It was diluted with DCM and the solution was filtered through celite. The filtrate was concentrated under vacuum to give the crude. The residue was purified by silica gel column chromatography (DCM:MeOH 30:1) to give the title compound (35 g, 76.4%) as a white solid.

LCMS (ESI) calcd for C₁₃H₁₅BrClN₃O [M+H]⁺ m/z=344.3, found 344.

### Step 3: N-(7-bromo-2-chloro-4-((2-hydroxy-2-methylpropyl)amino)quinolin-3-yl)-2-methylpentanamide

To a solution of 1-[(3-amino-7-bromo-2-chloroquinolin-4-yl) amino-2-methylpropan-2-ol (35 g, 101.56 mmol), and TEA (30.83 g, 304.67 mmol) in DCM (250 mL) was added 2-methylpentanoyl chloride (20.50 g, 152.33 mmol) dropwise under 0° C. Then the mixture was stirred at room temperature for 2 hours. LCMS showed the reaction was completed. The reaction was diluted with water and extracted with DCM (50 mL×3). The combined organic phases were washed with brine, dried over Na₂SO₄, concentrated to give the crude title compound (30 g, 67.2% yield) as yellow solid.

LCMS (ESI) calcd for C₁₉H₂₅BrClN₃O₂[M+H]⁺ m/z=442.1, found 442.

### Step 4: 1-(4-amino-7-bromo-2-(pentan-2-yl)-1H-imidazo[4,5-c]quinolin-1-yl)-2-methylpropan-2-ol

A solution of N-(7-bromo-2-chloro-4-((2-hydroxy-2-methylpropyl)amino)quinolin-3-yl)-2-methylpentanamide (30 g, 67.8 mmol) in NH₃-MeOH (100 mL) was stirred at 160° C. for 8 hours in a high-pressure reactor. After cooling to ambient temperature, the mixture was concentrated in vacuo. The residue was purified by silica gel column chromatography (DCM:MeOH 50:1) to afford the title compound (18 g, 65.5% yield) as a yellow solid.

LCMS (ESI) calcd for C₁₉H₂₅BrN₄O [M+H]⁺ m/z=405.34, found 405.

### Step 5: di-tert-butyl (7-bromo-1-(2-((tert-butoxycarbonyl)oxy)-2-methylpropyl)-2-(pentan-2-yl)-1H-imidazo[4,5-c]quinolin-4-yl)iminodicarbonate (13B)

To a solution of 1-(4-amino-7-bromo-2-(pentan-2-yl)-1H-imidazo[4,5-c]quinolin-1-yl)-2-methylpropan-2-ol (18 g, 44.41 mmol) in ACN (150 mL) was added DMAP (2.17 g, 17.76 mmol) and Boc₂O (96.92 g, 447.07 mmol). The reaction mixture was stirred at 75° C. for 4 hours. It was concentrated and diluted with water (200 mL) and extracted with EtOAc (150 mL×3). The combine organic phases were washed with brine, dried over Na₂SO₄, concentrated to give the crude. The residue was purified by silica gel column chromatography (PE:EA 25:1) to give the title compound (22 g, 70.26% yield) as a yellow solid.

LCMS (ESI) calcd for C₃₄H₄₉BrN₄O₇[M+H]⁺ m/z=705.6, found 705.3.

### Example 6

### Preparation of di-tert-butyl (7-bromo-1-(2-((tert-butoxycarbonyl) oxy)-2-methylpropyl)-2-butyl-1H-imidazo[4,5-c]quinoline-4-yl) iminodicarbonate (13C)

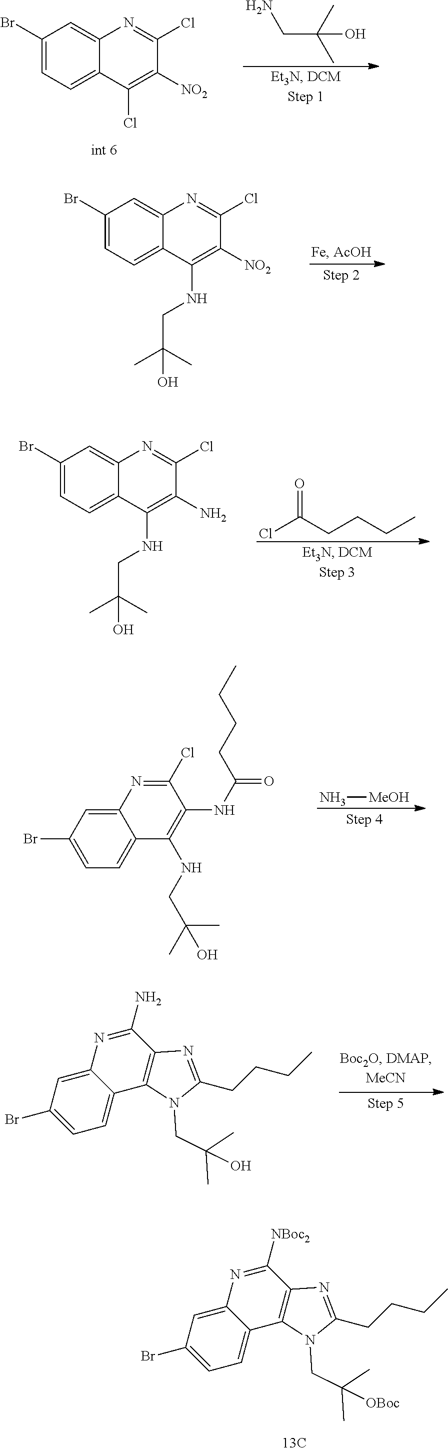

### Step 1: Preparation of 1-((7-bromo-2-chloro-3-nitroquinolin-4-yl) amino)-2-methylpropan-2-ol

To a solution of 7-bromo-2,4-dichloro-3-nitroquinoline (30 g, 93.2 mmol) and Et₃N (14.15 g, 139.8 mmol) in DCM (160 mL) was added 1-amino-2-methylpropan-2-ol (9.14 g, 102.5 mmol) dropwise at 0° C. The mixture was stirred rt for 4 hours. LCMS showed the reaction was completed. The resulting mixture was diluted with water (100 mL) and extracted with DCM (60 mL×3). The combine organic phases were washed with brine, dried over Na₂SO₄, concentrated to give the crude.

LCMS (ESI) calcd for C₁₃H₁₃BrClN₃O₃[M+H]⁺ m/z=374.4, found 374.

### Step 2: Preparation of 1-((3-amino-7-bromo-2-chloroquinolin-4-yl) amino)-2-methylpropan-2-ol

To a solution of 1-((7-bromo-2-chloro-3-nitroquinolin-4-yl) amino)-2-methylpropan-2-ol (32 g, 0.086 mol) in AcOH (200 mL) was added Fe (48 g, 0.86 mol) at RT. The mixture was stirred at RT for 6 hours. LCMS showed the reaction was completed. The resulting mixture was filtrated and concentrated in vacuo. The residue was diluted with water (100 mL) and extracted with EA (100 mL×3). The combine organic phases were washed with brine, dried over Na₂SO₄, concentrated to give the crude. The residue was purified by combi-flash (PE:EA 4:1) to afford 1-((3-amino-7-bromo-2-chloroquinolin-4-yl) amino)-2-methylpropan-2-ol (20 g, 67.96% yield) as yellow solid.

LCMS (ESI) calcd for C₁₃H₁₅BrClN₃O [M+H]⁺ m/z=344.3, found 344.

### Step 3: Preparation of N-(7-bromo-2-chloro-4-((2-hydroxy-2-methylpropyl) amino) quinolin-3-yl) pentanamide

To a solution of 1-((3-amino-7-bromo-2-chloroquinolin-4-yl) amino)-2-methylpropan-2-ol (20 g, 58.0 mmol) and Et₃N (8.8 g, 86.9 mmol) in DCM (40 mL) was added pentanoyl chloride (7.7 g, 63.9 mmol) dropwise at 0° C. Then the mixture was stirred at rt for 3 hours. LCMS showed the reaction was completed. The resulting mixture was diluted with water (100 mL) and extracted with DCM (100 mL×3). The combine organic phases were washed with brine, dried over Na₂SO₄, concentrated to give the crude. The residue was purified by combi-flash (PE:EA 3:1) to afford N-(7-bromo-2-chloro-4-((2-hydroxy-2-methylpropyl) amino) quinolin-3-yl) pentanamide (18 g, 72.32% yield) as yellow solid.

LCMS (ESI) calcd for C₁₈H₂₃BrClN₃O₂ [M+H]⁺ m/z=428.1, found 428.

### Step 4: Preparation of 1-(4-amino-7-bromo-2-butyl-1H-imidazo[4,5-c]quinolin-1-yl)-2-methylpropan-2-ol

A solution of N-(7-bromo-2-chloro-4-((2-hydroxy-2-methylpropyl) amino) quinolin-3-yl) pentanamide (18 g, 0.042 mol) in NH₃-MeOH (50 mL) was stirred at 160° C. for 8 hours in a high-pressure reactor. After cooling to ambient temperature, the resulting mixture was concentrated in vacuo. The residue was washed with PE:EA (1:1) to give 1-(4-amino-7-bromo-2-butyl-1H-imidazo[4,5-c]quinolin-1-yl)-2-methylpropan-2-ol (16 g, 97.3% yield) as yellow solid.

LCMS (ESI) calcd for C₁₈H₂₃BrN₄O [M+H]⁺ m/z=391.2, found 391.

### Step 5: Preparation of di-tert-butyl (7-bromo-1-(2-((tert-butoxycarbonyl)oxy)-2-methylpropyl)-2-butyl-1H-imidazo[4,5-c]quinolin-4-yl)iminodicarbonate (13C)

To a solution of 1-(4-amino-7-bromo-2-butyl-1H-imidazo[4,5-c]quinolin-1-yl)-2-methylpropan-2-ol (16 g, 0.041 mol) and DMAP (2 g, 0.016 mol) in MeCN (120 mL) was added Boc₂O (62.48 g, 0.286 mmol) at 75° C. The mixture was stirred at 75° C. for 4 hours. The resulting mixture was concentrated and then diluted with water (80 mL), extracted with EA (100 mL×3). The combine organic phases were washed with brine, dried over Na₂SO₄, concentrated to give the crude. The residue was purified by silica gel column chromatography (PE:EA 5:1) to afford di-tert-butyl (7-bromo-1-(2-((tert-butoxycarbonyl)oxy)-2-methylpropyl)-2-butyl-1H-imidazo[4,5-c]quinolin-4-yl)iminodicarbonate (18 g, 63.59% yield) as yellow solid.

LCMS (ESI) calcd for C₃₃H₄₇BrN₄O₇[M+H]⁺ m/z=691.4, found 691.

¹H NMR (400 MHz, DMSO-d₆) δ 8.56 (d, J=9.1 Hz, 1H), 8.21 (d, J=2.1 Hz, 1H), 7.75 (dd, J=9.1, 2.1 Hz, 1H), 3.02 (s, 2H), 1.90-1.79 (m, 2H), 1.52-1.41 (m, 4H), 1.38 (s, 3H), 1.29 (s, 21H), 1.21 (s, 9H), 0.94 (t, J=7.4 Hz, 3H).

### Example 7

### Synthesis of Compound 6

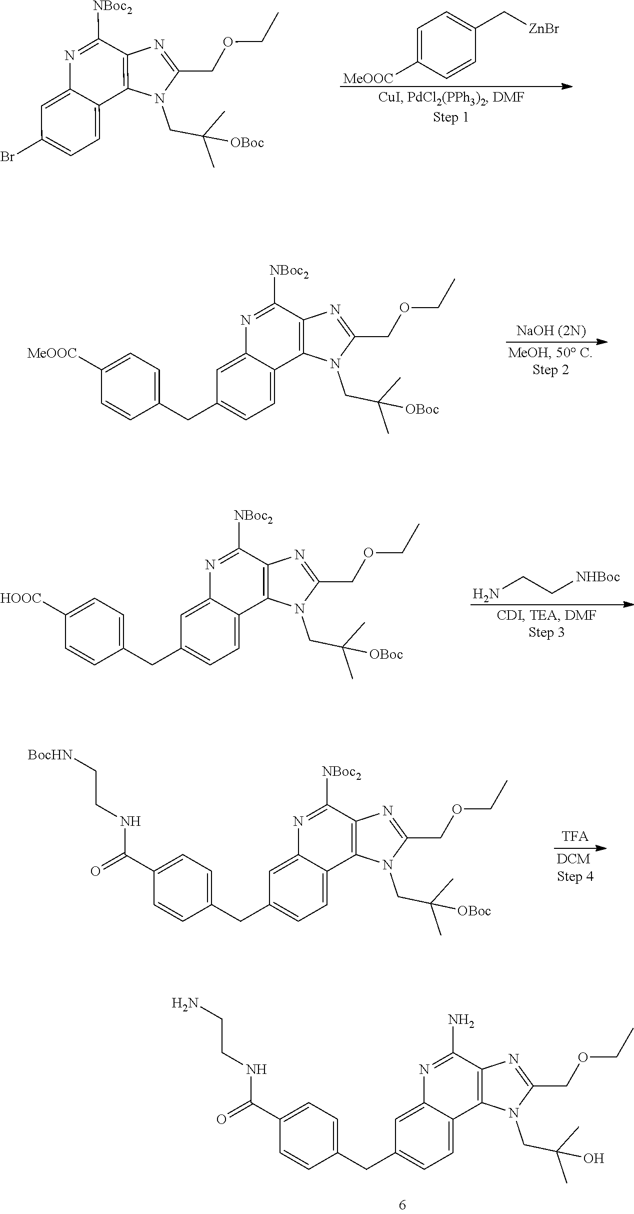

### Step 1: Preparation of di-tert-butyl (7-(4-methoxycarbonyl)benzyl)-1-(2-((tert-butoxycarbonyl)oxy)-2-methylpropyl)-2-(ethoxymethyl)-1H-imidazo[4,5-c]quinolin-4-yl)iminodicarbonate

To a solution of di-tert-butyl (7-bromo-1-(2-((tert-butoxycarbonyl) oxy)-2-methylpropyl)-2-(ethoxymethyl)-1H-imidazo[4,5-c]quinolin-4-yl) iminodicarbonate (693 mg, 1 mmol), Pd(PPh₃)Cl₂(81 mg, 0.12 mmol) and CuI (43 mg, 0.23 mmol) in DMF (10 mL) was added a solution of (4-(methoxycarbonyl)benzyl)zinc(II) bromide in DMF (0.6 mmol/mL, 15 mL). The mixture was stirred at 50° C. for 1 h. LCMS showed the reaction was completed. The resulting mixture was diluted with water (60 mL) and extracted with EA (40 mL×3). The combine organic phases were washed with brine, dried over Na₂SO₄, concentrated to give the crude. The residue was purified by silica gel column chromatography (PE:EA=1:1) to afford di-tert-butyl (7-(4-methoxycarbonyl)benzyl)-1-(2-((tert-butoxycarbonyl)oxy)-2-methylpropyl)-2-(ethoxymethyl)-1H-imidazo[4,5-c]quinolin-4-yl)iminodicarbonate (533 mg, 70% yield) as yellow solid.

### Step 2: Preparation of 4-((4-amino-2-(ethoxymethyl)-1-(2-hydroxy-2-methylpropyl)-1H-imidazo[4,5-c]quinolin-7-yl)methyl)benzoic acid

Di-tert-butyl (7-(4-methoxycarbonyl)benzyl)-1-(2-((tert-butoxycarbonyl)oxy)-2-methylpropyl)-2-(ethoxymethyl)-1H-imidazo[4,5-c]quinolin-4-yl)iminodicarbonate (553 mg, 0.73 mmol) in MeOH (5 mL) was added 2N NaOH (3 mL). The mixture was stirred at 50′C for 2 h. LCMS showed the reaction was completed. The resulting mixture was diluted with water (20 mL) and extracted with EA (10 mL×3). The combine organic phases were washed with brine, dried over Na₂SO₄, concentrated to give the crude. The residue was purified by silica gel column chromatography (PE:EA:HCOOH=1:1:0.01) to afford 4-((4-amino-2-(ethoxymethyl)-1-(2-hydroxy-2-methylpropyl)-1H-imidazo[4,5-c]quinolin-7-yl)methyl)benzoic acid (519 mg, 95% yield).

### Step 3: Preparation of di-tert-butyl (7-(4-((2-((tert-butoxycarbonyl)amino)ethyl)carbamoyl)benzyl)-1-(2-((tert-butoxycarbonyl)oxy)-2-methylpropyl)-2-(ethoxymethyl)-1H-imidazo[4,5-c]quinolin-4-yl)iminodicarbonate

To a solution of 4-((4-amino-2-(ethoxymethyl)-1-(2-hydroxy-2-methylpropyl)-1H-imidazo[4,5-c]quinolin-7-yl)methyl)benzoic acid (374 mg, 0.5 mmol), HOBT (81 mg, 0.6 mmol), and DIPEA (130 mg, 1 mmol) in DMF (5 mL) was added EDCI (114 mg, 0.6 mmol) at 0° C. The mixture was stirred at RT for overnight. LCMS showed the reaction was completed. The resulting mixture was diluted with water (20 mL) and extracted with EA (10 mL×3). The combine organic phases were washed with brine, dried over Na₂SO₄, concentrated to give the crude. The residue was purified by silica gel column chromatography (PE:EA=1:1) to afford di-tert-butyl (7-(4-((2-((tert-butoxycarbonyl)amino)ethyl)carbamoyl)benzyl)-1-(2-((tert-butoxycarbonyl)oxy)-2-methylpropyl)-2-(ethoxymethyl)-1H-imidazo[4,5-c]quinolin-4-yl)iminodicarbonate (294 mg, 66%).

### Step 4: Preparation of 4-((4-amino-2-(ethoxymethyl)-1-(2-hydroxy-2-methylpropyl)-1H-imidazo[4,5-c]quinolin-7-yl)methyl)-N-(2-aminoethyl)benzamide (Compound 6)

To a solution of di-tert-butyl (7-(4-((2-((tert-butoxycarbonyl)amino)ethyl)carbamoyl)benzyl)-1-(2-((tert-butoxycarbonyl)oxy)-2-methylpropyl)-2-(ethoxymethyl)-1H-imidazo[4,5-c]quinolin-4-yl)iminodicarbonate (294 mg, 0.33 mmol) in DCM (3 mL) was added TFA (1 mL) dropwise at 0° C. The mixture was stirred at RT for 1 h. The resulting mixture was concentrated in vacuo. The residue was adjusted to pH=8 with saturated sodium bicarbonate in aqueous solution. The resulting mixture was diluted with water (10 mL) and extracted with EA (10 mL×3). The combine organic phases were washed with brine, dried over Na₂SO₄, concentrated to give 4-((4-amino-2-(ethoxymethyl)-1-(2-hydroxy-2-methylpropyl)-1H-imidazo[4,5-c]quinolin-7-yl)methyl)-N-(2-aminoethyl)benzamide (150 mg, 94% yield).

LCMS (ESI) calcd for C₂₇H₃₄N₆O₃ [M+H]⁺ m/z=491.3, found 491.

¹H NMR (400 MHz, DMSO-d₆) δ 8.49-8.27 (m, 1H), 8.16 (d, J=8.5 Hz, 1H), 7.85-7.70 (m, 2H), 7.38 (dd, J=11.2, 4.9 Hz, 3H), 7.06 (d, J=8.5 Hz, 1H), 6.77 (s, 1H), 6.49 (s, 2H), 4.86 (s, 2H), 4.62 (s, 2H), 4.09 (s, 2H), 3.49 (q, J=7.0 Hz, 2H), 3.29-3.21 (m, 4H), 3.09 (q, J=6.1 Hz, 1H), 2.67 (t, J=6.5 Hz, 1H), 1.12 (t, J=7.0 Hz, 9H).

### Example 8

### Synthesis of Compound 7

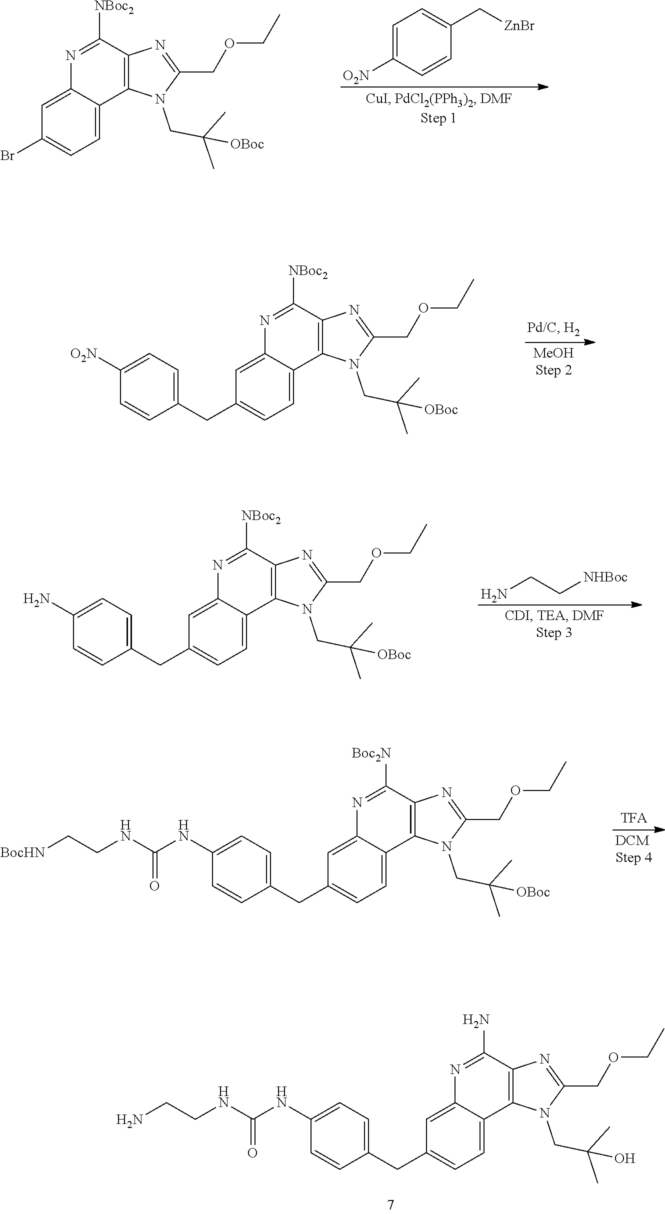

### Step 1: Preparation of di-tert-butyl (7-(4-nitro)benzyl)-1-(2-((tert-butoxycarbonyl)oxy)-2-methylpropyl)-2-(ethoxymethyl)-1H-imidazo[4,5-c]quinolin-4-yl)iminodicarbonate

To a solution of di-tert-butyl (7-bromo-1-(2-((tert-butoxycarbonyl) oxy)-2-methylpropyl)-2-(ethoxymethyl)-1H-imidazo[4,5-c]quinolin-4-yl) iminodicarbonate (693 mg, 1 mmol), Pd(PPh₃)Cl₂ (81 mg, 0.12 mmol) and CuI (43 mg, 0.23 mmol) in DMF (10 mL) was added a solution of (4-nitro-benzyl)zinc(II) bromide in DMF (15 mL). The mixture was stirred at 50′C for 1 h. LCMS showed the reaction was completed. The resulting mixture was diluted with water (60 mL) and extracted with EA (40 mL×3). The combine organic phases were washed with brine, dried over Na₂SO₄, concentrated to give the crude. The residue was purified by silica gel column chromatography (PE:EA=1:1) to afford di-tert-butyl (7-(4-nitro)benzyl)-1-(2-((tert-butoxycarbonyl)oxy)-2-methylpropyl)-2-(ethoxymethyl)-1H-imidazo[4,5-c]quinolin-4-yl)iminodicarbonate (555 mg, 75% yield) as yellow solid.

### Step 2: Preparation of di-tert-butyl (7-(4-amino)benzyl)-1-(2-((tert-butoxycarbonyl)oxy)-2-methylpropyl)-2-(ethoxymethyl)-1H-imidazo[4,5-c]quinolin-4-yl)iminodicarbonate

Di-tert-butyl (7-(4-nitro)benzyl)-1-(2-((tert-butoxycarbonyl)oxy)-2-methylpropyl)-2-(ethoxymethyl)-1H-imidazo[4,5-c]quinolin-4-yl)iminodicarbonate (555 mg, 0.75 mmol) in MeOH (10 mL) was added Pd/C catalyst (20 mg). The reaction was subjected with hydrogen balloon and stirred at room temperature for 8 hours. LCMS showed the reaction was completed. The resulting mixture was filtered and concentrated to give di-tert-butyl (7-(4-amino)benzyl)-1-(2-((tert-butoxycarbonyl)oxy)-2-methylpropyl)-2-(ethoxymethyl)-1H-imidazo[4,5-c]quinolin-4-yl)iminodicarbonate without purification.

### Step 3: di-tert-butyl (7-(4-(3-(2-((tert-butoxycarbonyl)amino)ethyl)ureido)benzyl)-1-(2-((tert-butoxycarbonyl)oxy)-2-methylpropyl)-2-(ethoxymethyl)-1H-imidazo[4,5-c]quinolin-4-yl)iminodicarbonate

di-tert-butyl (7-(4-amino)benzyl)-1-(2-((tert-butoxycarbonyl)oxy)-2-methylpropyl)-2-(ethoxymethyl)-1H-imidazo[4,5-c]quinolin-4-yl)iminodicarbonate (360 mg, 0.5 mmol), tert-butyl (2-aminoethyl)carbamate (96 mg, 0.6 mmol), and triethylamine (76 mg, 0.75 mmol) in DMF (5 mL) was added CDI (97 mg, 0.6 mmol) at 0° C. The mixture was stirred at RT for overnight. LCMS showed the reaction was completed. The resulting mixture was diluted with water (20 mL) and extracted with EA (10 mL×3). The combine organic phases were washed with brine, dried over Na₂SO₄, concentrated to give the crude. The residue was purified by silica gel column chromatography (PE:EA=1:1) to afford di-tert-butyl (7-(4-(3-(2-((tert-butoxycarbonyl)amino)ethyl)ureido)benzyl)-1-(2-((tert-butoxycarbonyl)oxy)-2-methylpropyl)-2-(ethoxymethyl)-1H-imidazo[4,5-c]quinolin-4-yl)iminodicarbonate (288 mg, 64%).

### Step 4: Preparation of 1-(4-((4-amino-2-(ethoxymethyl)-1-(2-hydroxy-2-methylpropyl)-1H-imidazo[4,5-c]quinolin-7-yl)methyl)phenyl)-3-(2-aminoethyl)urea

To a solution of di-tert-butyl (7-(4-(3-(2-((tert-butoxycarbonyl)amino)ethyl)ureido)benzyl)-1-(2-((tert-butoxycarbonyl)oxy)-2-methylpropyl)-2-(ethoxymethyl)-1H-imidazo[4,5-c]quinolin-4-yl)iminodicarbonate (288 mg, 0.32 mmol) in DCM (3 mL) was added TFA (1 mL) dropwise at 0° C. The mixture was stirred at RT for 1 h. The resulting mixture was concentrated in vacuo. The residue was adjusted to pH=8 with saturated sodium bicarbonate in aqueous solution. The resulting mixture was diluted with water (10 mL) and extracted with EA (10 mL×3). The combine organic phases were washed with brine, dried over Na₂SO₄, concentrated to give 1-(4-((4-amino-2-(ethoxymethyl)-1-(2-hydroxy-2-methylpropyl)-1H-imidazo[4,5-c]quinolin-7-yl)methyl)phenyl)-3-(2-aminoethyl)urea (155 mg, 96% yield).

LCMS (ESI) calcd for C₂₇H₃₅N₇O₃ [M+H]⁺ m/z=506.3, found 506.

### Example 9

### Synthesis of Compounds 8, 9 and 10

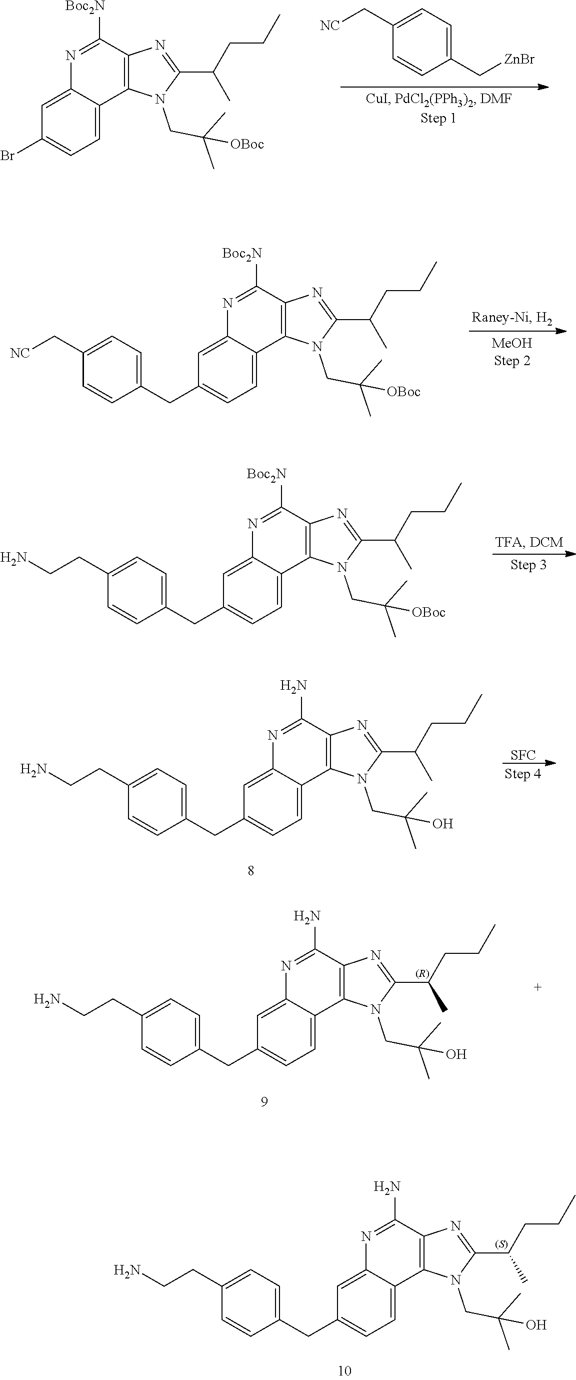

### Step 1: Preparation of di-tert-butyl (1-(2-((tert-butoxycarbonyl)oxy)-2-methylpropyl)-7-(4-(cyanomethyl)benzyl)-2-(pentan-2-yl)-1H-imidazo[4,5-c]quinolin-4-yl)iminodicarbonate

To a solution of di-tert-butyl (7-bromo-1-(2-((tert-butoxycarbonyl)oxy)-2-methylpropyl)-2-(pentan-2-yl)-1H-imidazo[4,5-c]quinolin-4-yl)iminodicarbonate (480 mg, 0.68 mmol), Copper(I) iodide (50.52 mg, 0.26 mmol] and PdCl₂(PPh₃)₂ (50.13 mg, 0.0714 mmol) in DMF (4 mL) was added (4-(cyanomethyl)benzyl)zinc(II) bromide (4 mL) stirred under nitrogen at room temperature. Then the mixture was stirred at 50° C. for 30 min. The reaction was quenched with H₂O, extracted with EA. The combined organic phases was washed with brine, dried over Na₂SO₄, filtered, and concentrated in vacuo. The residue was purified by silica gel column chromatography (PE:EA 10:1) to afford desired product (0.43 g, 79% yield) as a yellow solid.

LCMS (ESI) calcd for C₃₄H₄₉BrN₄O₇[M+H]⁺ m/z=756.43, found 756.55.

### Step 2: Preparation of di-tert-butyl (7-(4-(2-aminoethyl)benzyl)-1-(2-((tert-butoxycarbonyl)oxy)-2-methylpropyl)-2-(pentan-2-yl)-1H-imidazo[4,5-c]quinolin-4-yl)iminodicarbonate

A solution of di-tert-butyl (7-(4-(2-aminoethyl)benzyl)-1-(2-((tert-butoxycarbonyl)oxy)-2-methylpropyl)-2-(pentan-2-yl)-1H-imidazo[4,5-c]quinolin-4-yl)iminodicarbonate (150 mg, 0.20 mmol) and Raney-Ni (150 mg) in NH₃-MeOH (5 mL) was stirred under hydrogen at room temperature. The reaction mixture was stirred at room temperature for 3 h. The mixture was filtered through celite and the filtrate was concentrated to give the crude product (0.15 g, 99% yield).

LCMS (ESI) calcd for C₄₃H₆₁N₅O₇[M+H]⁺ m/z=760.46, found 760.45.

### Step 3: Preparation of 1-(4-amino-7-(4-(2-aminoethyl)benzyl)-2-(pentan-2-yl)-1H-imidazo[4,5-c]quinolin-1-yl)-2-methylpropan-2-ol (Compound 8)

To a solution of di-tert-butyl (1-(2-((tert-butoxycarbonyl)oxy)-2-methylpropyl)-7-(4-(cyanomethyl)benzyl)-2-(pentan-2-yl)-1H-imidazo[4,5-c]quinolin-4-yl)iminodicarbonate (150 mg, 0.19 mmol) in DCM (5 mL) was added TFA (1.5 mL) dropwise at room temperature. The reaction mixture was stirred at room temperature for 3 h. The solution was removed under reduced pressure to afford the crude product. The residue was purified by Prep-HPLC (column: Gemini-C18 150×21.2 mm, 5 um; mobile phase: ACN-MeOH (0.05% NH₃—H₂O); gradient: 25-60) to afford Compound 8 (79 mg, 84% yield) as white solid.

LCMS (ESI) calcd for C₂₈H₃₇N₅O [M+H]⁺ m/z=460.30, found 460.25.

¹HNMR (400 MHz, MeOD) δ 8.33 (d, J=8.7 Hz, 1H), 7.44 (d, J=1.5 Hz, 1H), 7.32 (dd, J=8.7, 1.7 Hz, 1H), 7.16 (m, 4H), 4.49 (m, 2H), 4.05 (s, 2H), 3.49-3.39 (m, 1H), 3.10-3.01 (m, 2H), 2.87-2.78 (m, 2H), 1.96-0.97 (m, 13H), 0.89-0.75 (m, 3H).

### Step 4: Preparation of (R)-1-(4-amino-7-(4-(2-aminoethyl) benzyl)-2-(pentan-2-yl)-1H-imidazo[4,5-c]quinolin-1-yl)-2-methylpropan-2-ol (Compound 9), (S)-1-(4-amino-7-(4-(2-aminoethyl)benzyl)-2-(pentan-2-yl)-1H-imidazo[4,5-c]quinolin-1-yl)-2-methylpropan-2-ol (Compound 10)

1-(4-amino-7-(4-(2-aminoethyl)benzyl)-2-(pentan-2-yl)-1H-imidazo[4,5-c]quinolin-1-yl)-2-methylpropan-2-ol (20 mg) was separated by SFC (Apparatus: LC-30AD SFC; Column: DAICEL AS-H 4.6 mml.D.*250 mmL, 5 μm; Mobile phase: CO₂/MeOH (0.1% NH₃)=80/20; Flow rate: 2.0 ml/min; Wave length: UV 214 nm & 254 nm; Temperature: 40° C.) to afford Compound 9 (4 mg, 99% purity) and Compound 10 (2 mg, 99% purity).

### Compound 9:

LCMS (ESI) calcd for C₂₈H₃₇N₅O [M+H]⁺ m/z=460.30, found 460.25.

¹H NMR (400 MHz, DMSO-d₆) δ 8.15 (d, J=8.6 Hz, 1H), 7.41-7.35 (m, 1H), 7.18 (d, J=8.0 Hz, 2H), 7.11 (d, J=8.0 Hz, 2H), 7.04 (dd, J=8.6, 1.8 Hz, 1H), 6.27 (s, 2H), 4.87-4.23 (m, 3H), 3.98 (s, 2H), 3.52-3.45 (m, 1H), 2.73 (t, J=7.1 Hz, 2H), 2.59 (t, J=7.2 Hz, 2H), 1.64-1.04 (m, 13H), 0.90-0.78 (m, 3H).

### Compound 10:

LCMS (ESI) calcd for C₂₈H₃₇N₅O [M+H]⁺ m/z=460.30, found 460.25.

¹H NMR (400 MHz, DMSO-d₆) δ 8.15 (d, J=8.6 Hz, 1H), 7.38 (s, 1H), 7.18 (d, J=8.0 Hz, 2H), 7.11 (d, J=8.0 Hz, 2H), 7.04 (dd, J=8.5, 1.7 Hz, 1H), 6.27 (s, 2H), 4.89-4.22 (m, 3H), 3.98 (s, 2H), 3.53-3.43 (m, 1H), 2.72 (t, J=7.3 Hz, 2H), 2.58 (t, J=7.2 Hz, 2H), 1.42-1.00 (m, 13H), 0.88-0.81 (m, 3H).

Compounds 1-5, 11-19 and 47 were prepared by similar method as described in Example 9 using corresponding reagents.

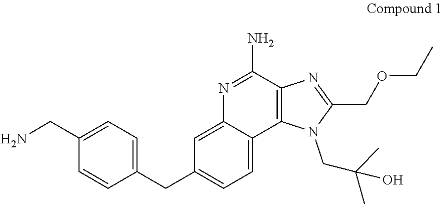 [SMILES: CCOCc1nc2c(N)nc3cc(Cc4ccc(CN)cc4)ccc3c2n1CC(C)(C)O]

### 1-(4-amino-7-(4-(aminomethyl)benzyl)-2-(ethoxymethyl)-1H-imidazo[4,5-c]quinolin-1-yl)-2-methylpropan-2-ol

LCMS (ESI) calcd for C₂₅H₃₁N₅O₂ [M+H]⁺ m/z=434.2, found 434.0.

¹H NMR (400 MHz, CD₃OD, ppm) δ 8.49 (bs, 2H), 8.28 (d, J=8.6 Hz, 1H), 7.53 (s, 1H), 7.42-7.35 (m, 4H), 7.28 (d, J=8.5 Hz, 1H), 5.00 (bs, 2H), 4.78 (s, 2H), 4.15 (s, 2H), 4.08 (s, 2H), 3.62 (q, J=6.9 Hz, 2H), 1.27-1.22 (m, 9H).

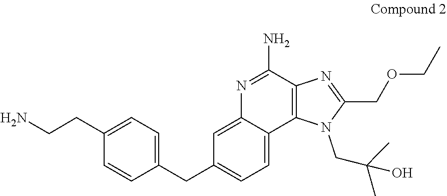 [SMILES: CCOCc1nc2c(N)nc3cc(Cc4ccc(CCN)cc4)ccc3c2n1CC(C)(C)O]

### 1-(4-amino-7-(4-(2-aminoethyl)benzyl)-2-(ethoxymethyl)-1H-imidazo[4,5-c]quinolin-1-yl)-2-methylpropan-2-ol

LCMS (ESI) calcd for C₂₆H₃₃N₅O₂ [M+H]⁺ m/z=448.3, found 448.0.

¹H NMR (400 MHz, CD₃OD, ppm) δ 8.47 (s, 2H), 8.35 (d, J=8.6 Hz, 1H), 7.57 (s, 1H), 7.38 (d, J=9.6 Hz, 1H), 7.31 (d, J=8.0 Hz, 2H), 7.25 (d, J=8.1 Hz, 2H), 5.02 (s, 1H), 4.81 (s, 2H), 4.15 (s, 2H), 3.64 (q, J=7.0 Hz, 2H), 3.24-3.13 (m, 2H), 3.03-2.88 (m, 2H), 1.29-1.24 (m, 9H).

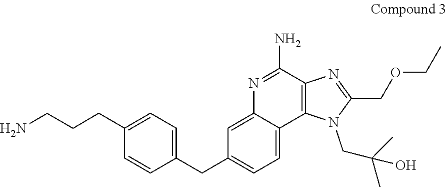 [SMILES: CCOCc1nc2c(N)nc3cc(Cc4ccc(CCCN)cc4)ccc3c2n1CC(C)(C)O]

### 1-(4-amino-7-(4-(3-aminopropyl)benzyl)-2-(ethoxymethyl)-1H-imidazo[4,5-c]quinolin-1-yl)-2-methylpropan-2-ol

LCMS (ESI) calcd for C₂₇H₃₅N₅O₂ [M+H]⁺ m/z=461.6, found 462.

¹H NMR (400 MHz, DMSO-d₆) δ 8.30 (s, 2H), 8.16 (d, J=8.6 Hz, 1H), 7.38 (d, J=1.7 Hz, 1H), 7.22 (d, J=8.0 Hz, 2H), 7.14 (d, J=8.1 Hz, 2H), 7.07 (dd, J=8.5, 1.8 Hz, 1H), 6.57 (s, 2H), 4.87 (s, 2H), 4.63 (s, 2H), 4.00 (s, 2H), 3.50 (q, J=7.0 Hz, 3H), 2.83-2.70 (m, 2H), 2.60 (t, J=7.7 Hz, 2H), 1.97-1.61 (m, 2H), 1.12 (t, J=7.0 Hz, 8H).

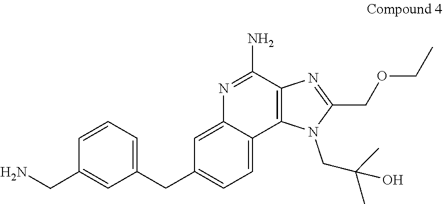 [SMILES: CCOCc1nc2c(N)nc3cc(Cc4cccc(CN)c4)ccc3c2n1CC(C)(C)O]

### 1-(4-amino-7-(3-(aminomethyl)benzyl)-2-(ethoxymethyl)-1H-imidazo[4,5-c]quinolin-1-yl)-2-methylpropan-2-ol

LCMS (ESI) calcd for C₂₅H₃₁N₅O₂[M+H]⁺ m/z=434.3, found 434.

¹H NMR (400 MHz, DMSO-d₆) δ 13.85 (s, 1H), 8.83 (s, 2H), 8.39 (d, J=8.5 Hz, 1H), 8.19 (s, 2H), 7.57 (s, 1H), 7.44-7.28 (m, 5H), 4.93 (s, 2H), 4.69 (s, 2H), 4.14 (s, 2H), 4.01 (s, 2H), 3.54 (q, J=7.0 Hz, 2H), 1.32-0.97 (m, 9H).

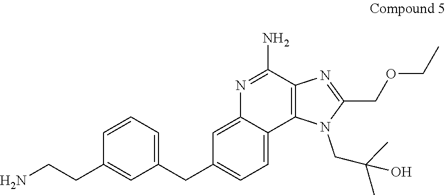 [SMILES: CCOCc1nc2c(N)nc3cc(Cc4cccc(CCN)c4)ccc3c2n1CC(C)(C)O]

### 1-(4-amino-7-(3-(2-aminoethyl)benzyl)-2-(ethoxymethyl)-1H-imidazo[4,5-c]quinolin-1-yl)-2-methylpropan-2-ol

LCMS (ESI) calcd for C₂₆H₃₃N₅O₂ [M+H]⁺ m/z=448.3, found 448.

¹H NMR (400 MHz, DMSO-d6) δ 8.53 (s, 2H), 8.37 (d, J=8.6 Hz, 1H), 7.91 (s, 2H), 7.53 (s, 1H), 7.30 (t, J=7.3 Hz, 2H), 7.23-7.05 (m, 3H), 4.94 (s, 2H), 4.69 (s, 2H), 4.10 (s, 2H), 3.53 (q, J=7.0 Hz, 2H), 3.07-3.00 (m, 2H), 2.89-2.79 (m, 2H), 1.31-1.05 (m, 9H).

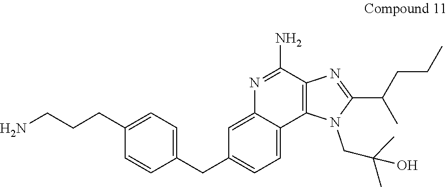 [SMILES: CCCC(C)c1nc2c(N)nc3cc(Cc4ccc(CCCN)cc4)ccc3c2n1CC(C)(C)O]

### 1-(4-amino-7-(4-(3-aminopropyl)benzyl)-2-(pentan-2-yl)-1H-imidazo[4,5-c]quinolin-1-yl)-2-methylpropan-2-ol

LCMS (ESI) calcd for C₂₉H₃₉N₅O [M+H]⁺ m/z=474.5, found 474.

¹H NMR (400 MHz, DMSO-d₆) δ 13.65 (s, 1H), 8.79 (s, 2H), 8.42 (d, J=8.7 Hz, 1H), 7.75 (s, 3H), 7.56 (s, 1H), 7.35 (d, J=8.6 Hz, 1H), 7.19 (m, 4H), 4.79 (s, 1H), 4.09 (s, 2H), 3.58-3.44 (m, 1H), 2.78 (m, 2H), 2.67-2.55 (m, 2H), 1.81 (m, 2H), 1.64-0.54 (m, 16H).

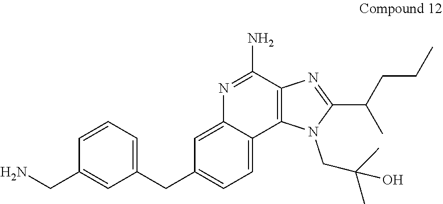 [SMILES: CCCC(C)c1nc2c(N)nc3cc(Cc4cccc(CN)c4)ccc3c2n1CC(C)(C)O]

### 1-(4-amino-7-(3-(aminomethyl)benzyl)-2-(pentan-2-yl)-1H-imidazo[4,5-c]quinolin-1-yl)-2-methylpropan-2-ol

LCMS (ESI) calcd for C₂₇H₃₅N₅O [M+H]⁺ m/z=446.3, found 446.0.

¹H NMR (400 MHz, DMSO-d₆, ppm) δ 13.46 (s, 1H), 8.73 (s, 1H), 8.42 (d, J=8.5 Hz, 1H), 8.14 (s, 3H), 7.58 (s, 1H), 7.48-7.27 (m, 5H), 4.77 (s, 1H), 4.14 (s, 2H), 4.01 (s, 2H), 3.49 (dd, J=13.8, 7.0 Hz, 1H), 2.01-0.60 (m, 16H).

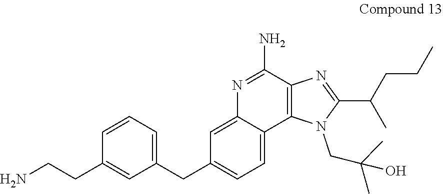 [SMILES: CCCC(C)c1nc2c(N)nc3cc(Cc4cccc(CCN)c4)ccc3c2n1CC(C)(C)O]

### 1-(4-amino-7-(3-(2-aminoethyl)benzyl)-2-(pentan-2-yl)-1H-imidazo[4,5-c]quinolin-1-yl)-2-methylpropan-2-ol

LCMS (ESI) calcd for C₂₈H₃₇N₅O [M+H]⁺ m/z=460.3, found 460.0.

¹H NMR (400 MHz, DMSO-d₆, ppm) δ 8.34 (d, J=8.1 Hz, 1H), 7.80 (s, 2H), 7.51 (s, 1H), 7.28 (q, J=7.8 Hz, 2H), 7.15 (dd, J=21.9, 8.0 Hz, 3H), 4.76 (s, 1H), 4.48 (s, 1H), 4.08 (s, 2H), 3.49 (dd, J=13.7, 6.9 Hz, 1H), 3.09-2.95 (m, 2H), 2.90-2.73 (m, 2H), 1.87-0.67 (m, 16H).

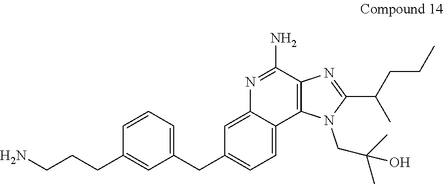 [SMILES: CCCC(C)c1nc2c(N)nc3cc(Cc4cccc(CCCN)c4)ccc3c2n1CC(C)(C)O]

### 1-(4-amino-7-(3-(3-aminopropyl)benzyl)-2-(pentan-2-yl)-1H-imidazo[4,5-c]quinolin-1-yl)-2-methylpropan-2-ol

LCMS (ESI) calcd for C₂₉H₃₉N₅O [M+H]⁺ m/z=474.3, found 474.

¹H NMR (400 MHz, DMSO-d₆) δ 8.20 (d, J=8.5 Hz, 1H), 8.15 (s, 1H), 7.66 (s, 2H), 7.41 (s, 1H), 7.25 (t, J=7.5 Hz, 1H), 7.16-7.04 (m, 4H), 6.71 (s, 2H), 4.73 (s, 1H), 4.48 (s, 2H), 4.02 (s, 2H), 3.48 (dd, J=13.8, 7.0 Hz, 2H), 2.87-2.73 (m, 2H), 2.67-2.54 (m, 2H), 1.83-1.76 (m, 2H), 1.61 (s, 2H), 1.42-1.13 (m, 10H), 0.85 (s, 3H).

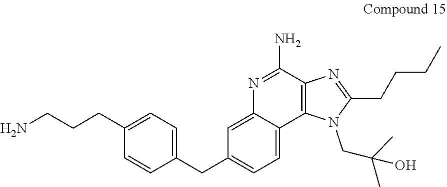 [SMILES: CCCCc1nc2c(N)nc3cc(Cc4ccc(CCCN)cc4)ccc3c2n1CC(C)(C)O]

### 1-(4-amino-7-(4-(3-aminopropyl)benzyl)-2-butyl-1H-imidazo[4,5-c]quinolin-1-yl)-2-methylpropan-2-ol

LCMS (ESI) calcd for C₂₈H₃₇N₅O [M+H]⁺ m/z=460.3, found 460.

¹H NMR (400 MHz, DMSO-d₆) δ 8.29 (s, 1H), 7.64 (s, 2H), 7.48 (s, 1H), 7.23 (d, J=8.0 Hz, 2H), 7.15 (d, J=8.1 Hz, 2H), 4.78 (s, 1H), 4.05 (s, 2H), 3.02 (t, J=7.6 Hz, 2H), 2.78 (s, 2H), 2.60 (t, J=7.7 Hz, 2H), 1.80 (s, 4H), 1.42 (dd, J=15.0, 7.7 Hz, 2H), 1.23 (s, 8H), 1.16 (s, 4H), 0.94 (t, J=7.3 Hz, 3H).

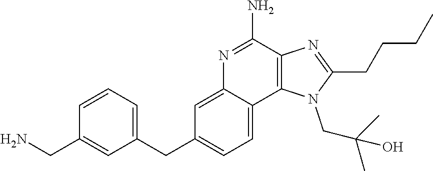 [SMILES: CCCCc1nc2c(N)nc3cc(Cc4cccc(CN)c4)ccc3c2n1CC(C)(C)O]

### 1-(4-amino-7-(3-(aminomethyl)benzyl)-2-butyl-1H-imidazo[4,5-c]quinolin-1-yl)-2-methylpropan-2-ol

LCMS (ESI) calcd for C₂₆H₃₃N₅O [M+H]⁺ m/z=432.3, found 432.

¹H NMR (400 MHz, DMSO-d₆) δ 8.26-8.14 (m, 2H), 7.45-7.29 (m, 5H), 7.09 (d, J=8.5 Hz, 1H), 6.69 (s, 2H), 4.82-4.37 (m, 2H), 4.02 (d, J=18.9 Hz, 4H), 3.39 (s, 2H), 3.04-2.89 (m, 2H), 1.83-1.69 (m, 2H), 1.44-1.36 (m, 2H), 1.15 (s, 6H), 0.93 (t, J=7.4 Hz, 3H).

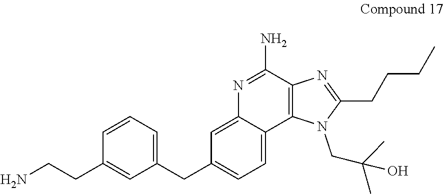 [SMILES: CCCCc1nc2c(N)nc3cc(Cc4cccc(CCN)c4)ccc3c2n1CC(C)(C)O]

### 1-(4-amino-7-(3-(2-aminoethyl)benzyl)-2-butyl-1H-imidazo[4,5-c]quinolin-1-yl)-2-methylpropan-2-ol

LCMS (ESI) calcd for C₂₇H₃₅N₅O [M+H]⁺ m/z=446.3, found 446.0.

¹H NMR (400 MHz, DMSO-d₆, ppm) δ 8.35 (d, J=8.7 Hz, 1H), 7.81 (s, 2H), 7.52 (s, 1H), 7.30 (t, J=7.5 Hz, 2H), 7.15 (dd, J=21.2, 8.0 Hz, 3H), 4.79 (s, 1H), 4.09 (s, 2H), 3.17-2.94 (m, 4H), 2.93-2.73 (m, 2H), 1.95-1.69 (m, 2H), 1.42 (dd, J=14.9, 7.4 Hz, 2H), 1.17 (s, 5H), 0.94 (t, J=7.4 Hz, 3H).

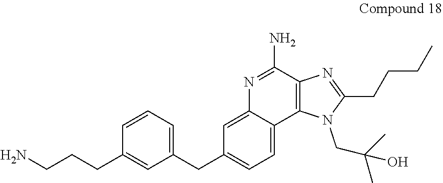 [SMILES: CCCCc1nc2c(N)nc3cc(Cc4cccc(CCCN)c4)ccc3c2n1CC(C)(C)O]

### 1-(4-amino-7-(3-(3-aminopropyl)benzyl)-2-butyl-1H-imidazo[4,5-c]quinolin-1-yl)-2-methylpropan-2-ol

LCMS (ESI) calcd for C₂₈H₃₇N₅O [M+H]⁺ m/z=460.3, found 460.

¹H NMR (400 MHz, DMSO-d₆) δ 8.21 (s, 1H), 8.16 (d, J=8.4 Hz, 1H), 7.39 (s, 1H), 7.24 (t, J=7.6 Hz, 1H), 7.13 (d, J=7.4 Hz, 2H), 7.06 (t, J=7.7 Hz, 2H), 6.58 (s, 2H), 4.48 (s, 2H), 4.01 (s, 2H), 3.05-2.91 (m, 2H), 2.85-2.73 (m, 2H), 2.60 (t, J=7.7 Hz, 2H), 1.79 (m, 4H), 1.49-1.30 (m, 2H), 1.15 (s, 6H), 0.93 (t, J=7.4 Hz, 3H).

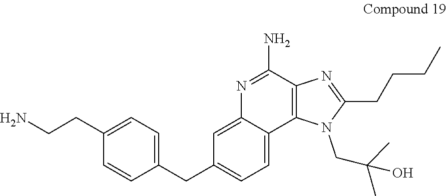 [SMILES: CCCCc1nc2c(N)nc3cc(Cc4ccc(CCN)cc4)ccc3c2n1CC(C)(C)O]

### 1-(4-amino-7-(4-(2-aminoethyl)benzyl)-2-butyl-1H-imidazo[4,5-c]quinolin-1-yl)-2-methylpropan-2-ol

LCMS (ESI) calcd for C₂₇H₃₅N₅O [M+H]⁺ m/z=446.2, found 446.

¹H NMR (400 MHz, DMSO-d₆) δ 8.18 (d, J=8.5 Hz, 1H), 8.14 (s, 1H), 7.73 (s, 2H), 7.40 (s, 1H), 7.26 (d, J=8.0 Hz, 2H), 7.19 (d, J=8.0 Hz, 2H), 7.10 (d, J=9.0 Hz, 1H), 6.61 (s, 1H), 4.76 (s, 1H), 4.52 (s, 1H), 4.01 (s, 2H), 3.01 (dd, J=14.4, 7.1 Hz, 4H), 2.90-2.73 (m, 2H), 1.87-1.69 (m, 2H), 1.50-1.35 (m, 2H), 0.99-1.34 (m, 9H).

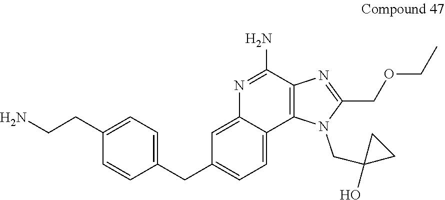 [SMILES: CCOCc1nc2c(N)nc3cc(Cc4ccc(CCN)cc4)ccc3c2n1CC1(O)CC1]

### 1-((4-amino-7-(4-(2-aminoethyl)benzyl)-2-(ethoxymethyl)-1H-imidazo[4,5-c]quinolin-1-yl)methyl)cyclopropan-1-ol

LCMS (ESI) calcd for C₂₆H₃₁N₅O₂ [M+H]⁺ m/z=446.5, found 446.

¹H NMR (400 MHz, DMSO-d₆) δ 9.08 (s, 1H), 8.07-7.68 (m, 4H), 7.60 (s, 1H), 7.34 (d, J=8.5 Hz, 1H), 7.23 (dd, J=19.7, 8.1 Hz, 4H), 5.74 (s, 2H), 4.77 (s, 2H), 4.11 (s, 2H), 3.38 (dd, J=13.9, 6.9 Hz, 2H), 3.01 (s, 2H), 2.89-2.69 (m, 4H), 1.02 (q, J=7.2 Hz, 6H).

### Example 10

### Synthesis of Compound 20

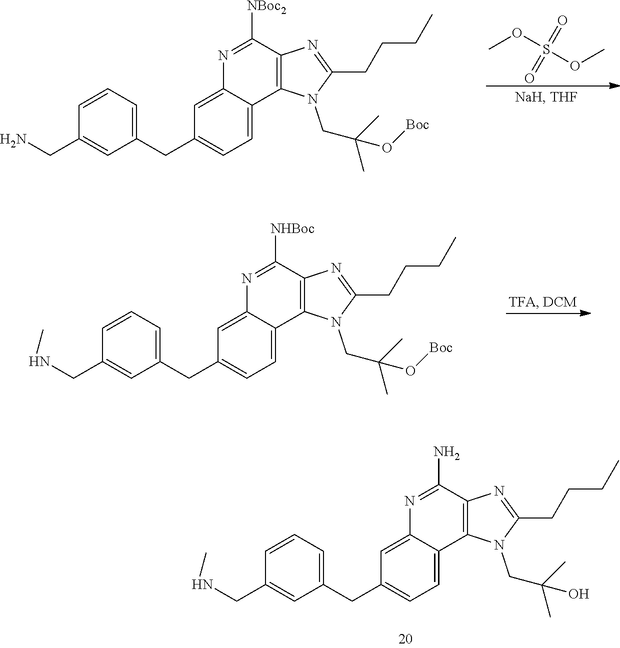

### Step 1. Preparation of tert-butyl (1-(2-((tert-butoxycarbonyl)oxy)-2-methylpropyl)-2-butyl-7-(3-((methylamino)methyl)benzyl)-1H-imidazo[4,5-c]quinolin-4-yl)carbamate

To a solution of di-tert-butyl (7-(3-(aminomethyl)benzyl)-1-(2-((tert-butoxycarbonyl)oxy)-2-methylpropyl)-2-butyl-1H-imidazo[4,5-c]quinolin-4-yl)iminodicarbonate (100 mg, 0.14 mmol) in THF (3 mL) was added NaH (11 mg, 0.27 mmol, 60% in oil) at 0° C. The mixture was stirred at 0° C. for 0.5 h. Then dimethyl sulfate (17.2 mg, 0.14 mmol) was added to the mixture and warmed to rt for overnight. LCMS showed the TM. The resulting mixture was quenched with water (20 mL) and extracted with EA (20 mL×3). The combine organic phases were washed with brine, dried over Na₂SO₄, concentrated to give the crude. The residue was purified by combi-flash (PE:EA 1:1) to afford the product (10 mg, 9.6% yield) as yellow solid.

LCMS (ESI) calcd for C₃₉H₅₂N₄O₈ [M+H]⁺ m/z=746.44, found 646.

### Step 2. Preparation of 1-(4-amino-2-butyl-7-(3-((methylamino)methyl)benzyl)-1H-imidazo[4,5-c]quinolin-1-yl)-2-methylpropan-2-ol (Compound 20)

To a solution of di-tert-butyl (1-(2-((tert-butoxycarbonyl)oxy)-2-methylpropyl)-2-butyl-7-(3-((methylamino)methyl)benzyl)-11H-imidazo[4,5-c]quinolin-4-yl)iminodicarbonate (10 mg, 0.015 mmol) in DCM (1 mL) was added TFA (0.3 mL) dropwise at 0° C. The mixture was stirred at RT for 2 hours. The resulting mixture was concentrated in vacuo. The residue was purified by Prep-HPLC (Column: Gemini-C18,150×21.2 mm, 5 um; Mobile Phase: ACN-H2O (0.1% FA), Gradient: 15-45) to give Compound 20 (1.8 mg, 25% yield) as white solid.

LCMS (ESI) calcd for C₂₇H₃₅N₅O [M+H]⁺ m/z=446.28, found 446.

¹H-NMR (400 MHz, DMSO-d₆) δ 8.31-7.99 (m, 1H), 7.52-7.18 (m, 6H), 7.18 (s, 2H), 4.77 (s, 1H), 4.08 (s, 4H), 3.01 (m, 2H), 1.90-1.48 (m, 2H), 1.48 (m, 2H), 1.16 (s, 6H), 0.93 (t, J=7.3 Hz, 3H).

Compounds 51-55 and 67-71 were prepared by similar method as described in Example 6 and Example 9 using corresponding reagents. The general scheme for preparing Compounds 51-55 and 67-71 is shown as below:

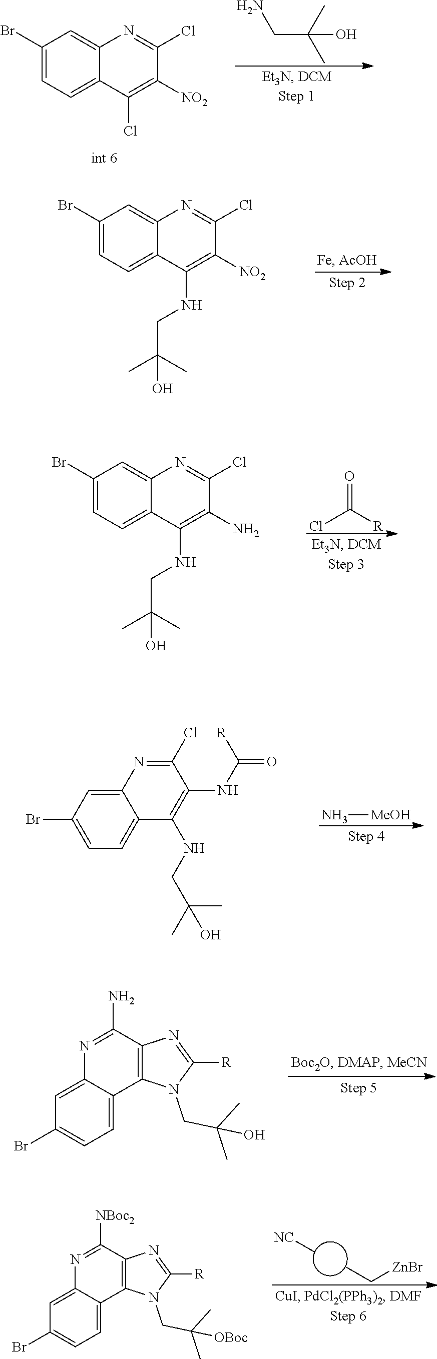

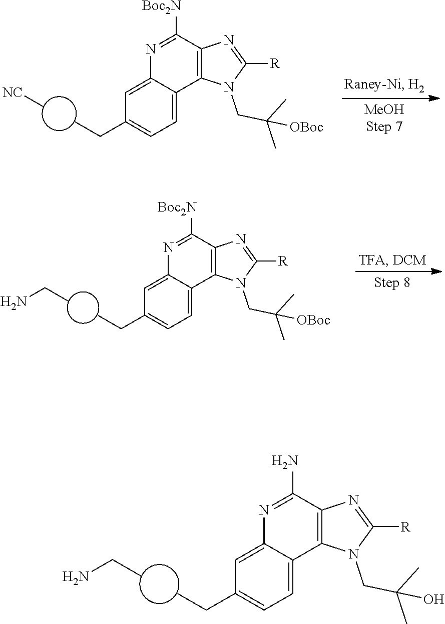

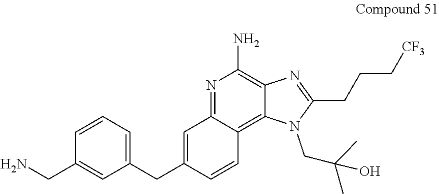 [SMILES: CC(C)(O)Cn1c(CCCC(F)(F)F)nc2c(N)nc3cc(Cc4cccc(CN)c4)ccc3c21]

### 1-(4-amino-7-(3-(aminomethyl)benzyl)-2-(4,4,4-trifluorobutyl)-1H-imidazo[4,5-c]quinolin-1-yl)-2-methylpropan-2-ol

LCMS (ESI) calcd for C₂₆H₃₁F₃N₅O [M+H]⁺ m/z=486.25, found 486.15.

¹H NMR (400 MHz, MeOD, ppm) δ 8.43 (d, J=8.6 Hz, 1H), 7.61 (s, 1H), 7.40 (m, 5H), 4.67 (s, 2H), 4.23 (s, 2H), 4.11 (s, 2H), 3.19 (s, 2H), 2.54-2.39 (m, 2H), 2.32-2.16 (m, 2H), 1.31 (s, 6H).

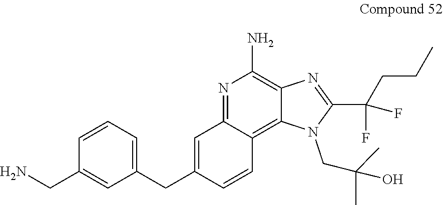 [SMILES: CCCC(F)(F)c1nc2c(N)nc3cc(Cc4cccc(CN)c4)ccc3c2n1CC(C)(C)O]

### 1-(4-amino-7-(3-(aminomethyl)benzyl)-2-(1,1-difluorobutyl)-1H-imidazo[4,5-c]quinolin-1-yl)-2-methylpropan-2-ol

LCMS (ESI) calcd for C₂₆H₃₂F₂N₅O [M+H]⁺ m/z=468.26, found 468.20.

¹H NMR (400 MHz, DMSO-d₆, ppm) δ 14.21 (s, 1H), 9.26 (s, 2H), 8.56 (d, J=8.7 Hz, 1H), 8.23 (s, 3H), 7.59 (s, 1H), 7.47-7.03 (m, 5H), 4.98 (d, J=15.3 Hz, 1H), 4.58 (d, J=15.3 Hz, 1H), 4.28-3.87 (m, 4H), 2.58 (m, 2H), 1.68-1.39 (m, 2H), 1.28 (s, 3H), 1.01 (m, 6H).

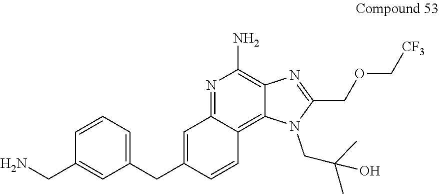 [SMILES: CC(C)(O)Cn1c(COCC(F)(F)F)nc2c(N)nc3cc(Cc4cccc(CN)c4)ccc3c21]

### 1-(4-amino-7-(3-(aminomethyl)benzyl)-2-((2,2,2-trifluoroethoxy)methyl)-1H-imidazo[4,5-c]quinolin-1-yl)-2-methylpropan-2-ol

LCMS (ESI) calcd for C₂₅H₂₉F₃N₅O₂[M+H]⁺ m/z=488.23, found 488.20.

¹H NMR (400 MHz, DMSO-d₆, ppm) δ 13.68 (s, 1H), 8.98 (s, 1H), 8.43 (d, J=8.6 Hz, 1H), 8.11 (s, 3H), 7.59 (s, 1H), 7.38 (m, 5H), 5.13 (s, 1H), 4.96 (s, 1H), 4.69 (s, 2H), 4.26 (q, J=9.3 Hz, 2H), 4.19-4.08 (m, 2H), 4.01 (d, J=5.1 Hz, 2H), 1.41-1.02 (m, 6H).

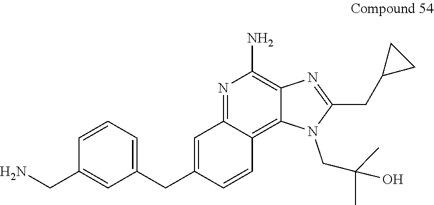 [SMILES: CC(C)(O)Cn1c(CC2CC2)nc2c(N)nc3cc(Cc4cccc(CN)c4)ccc3c21]

### 1-(4-amino-7-(3-(aminomethyl)benzyl)-2-(cyclopropylmethyl)-1H-imidazo[4,5-c]quinolin-1-yl)-2-methylpropan-2-ol

LCMS (ESI) calcd. for C₂₆H₃₁N₅O [M+H]⁺ m/z 430.26, found 430.20.

¹H NMR (400 MHz, DMSO-d₆, ppm) δ 8.23 (s, 1H), 8.16 (d, J=8.6 Hz, 1H), 7.44-7.26 (m, 5H), 7.09 (d, J=8.5 Hz, 1H), 6.57 (s, 2H), 4.52 (s, 2H), 4.01 (d, J=25.7 Hz, 4H), 2.99 (s, 2H), 1.40-0.71 (m, 7H), 0.49 (d, J=7.9 Hz, 2H), 0.25 (d, J=4.8 Hz, 2H).

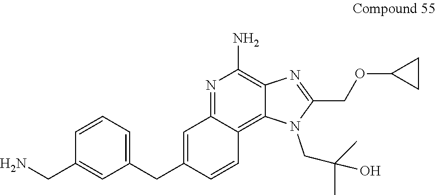 [SMILES: CC(C)(O)Cn1c(COC2CC2)nc2c(N)nc3cc(Cc4cccc(CN)c4)ccc3c21]

### 1-(4-amino-7-(3-(aminomethyl)benzyl)-2-(cyclopropoxymethyl)-1H-imidazo[4,5-c]quinolin-1-yl)-2-methylpropan-2-ol

LCMS (ESI) calcd for C₂₆H₃₂N₅O₂ [M+H]⁺ m/z=446.26, found 446.20.

¹H NMR (400 MHz, MeOD) δ 8.32 (d, J=8.6 Hz, 1H), 7.53 (s, 1H), 7.39-7.16 (m, 5H), 4.97 (s, 2H), 4.68 (s, 2H), 4.10 (s, 2H), 4.01 (s, 2H), 3.40-3.32 (m, 1H), 1.19 (s, 6H), 0.64-0.31 (m, 4H).

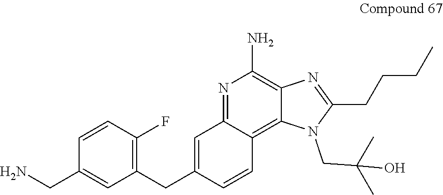 [SMILES: CCCCc1nc2c(N)nc3cc(Cc4cc(CN)ccc4F)ccc3c2n1CC(C)(C)O]

### 1-(4-amino-7-(5-(aminomethyl)-2-fluorobenzyl)-2-butyl-1H-imidazo[4,5-c]quinolin-1-yl)-2-methylpropan-2-ol

LCMS (ESI) calcd for C₂₆H₃₂FN₅O [M+H]⁺ m/z=450.3, found 450.3.

¹H NMR (400 MHz, DMSO-d6, ppm) δ 13.88 (s, 1H), 8.92 (s, 2H), 8.42 (d, J=8.7 Hz, 1H), 8.21 (s, 3H), 7.59 (s, 1H), 7.50 (dd, J=7.2, 2.0 Hz, 1H), 7.45-7.38 (m, 1H), 7.31 (dd, J=17.9, 8.4 Hz, 2H), 4.82 (s, 2H), 4.15 (s, 2H), 4.01 (s, 2H), 3.04 (t, J=7.7 Hz, 2H), 1.86-1.75 (m, 2H), 1.42 (dd, J=14.9, 7.4 Hz, 2H), 1.18 (s, 6H), 0.94 (t, J=7.4 Hz, 3H).

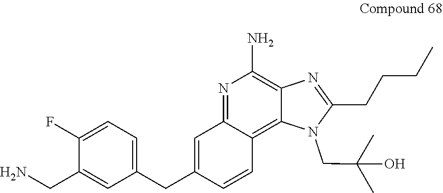 [SMILES: CCCCc1nc2c(N)nc3cc(Cc4ccc(F)c(CN)c4)ccc3c2n1CC(C)(C)O]

### 1-(4-amino-7-(3-(aminomethyl)-4-fluorobenzyl)-2-butyl-1H-imidazo[4,5-c]quinolin-1-yl)-2-methylpropan-2-ol

LCMS (ESI) calcd for C₂₆H₃₂FN₅O [M+H]⁺ m/z=450.3, found 450.2.

¹H NMR (400 MHz, DMSO-d6, ppm) δ 13.74 (s, 1H), 8.90 (s, 1H), 8.42 (d, J=8.7 Hz, 1H), 8.24 (s, 3H), 7.56 (s, 1H), 7.45 (dd, J=7.2, 2.0 Hz, 1H), 7.42-7.36 (m, 1H), 7.34 (d, J=8.6 Hz, 1H), 7.31-7.23 (m, 1H), 4.82 (s, 2H), 4.12 (s, 2H), 4.05 (d, J=5.3 Hz, 2H), 3.04 (t, J=7.6 Hz, 2H), 1.87-1.74 (m, 2H), 1.49-1.36 (m, 2H), 1.18 (s, 6H), 0.94 (t, J=7.4 Hz, 3H).

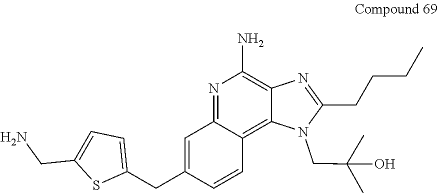 [SMILES: CCCCc1nc2c(N)nc3cc(Cc4ccc(CN)s4)ccc3c2n1CC(C)(C)O]

### 1-(4-amino-7-((5-(aminomethyl)thiophen-2-yl)methyl)-2-butyl-1H-imidazo[4,5-c]quinolin-1-yl)-2-methylpropan-2-ol

LCMS (ESI) calcd for C₂₄H₃₁N₅OS [M+H]⁺ m/z=438.22, found 438.3.

¹H NMR (400 MHz, DMSO-d6, ppm) δ 13.71 (s, 1H), 8.90 (s, 1H), 8.45 (d, J=8.7 Hz, 1H), 8.16 (s, 3H), 7.63 (s, 1H), 7.39 (d, J=8.6 Hz, 1H), 7.07 (d, J=3.4 Hz, 1H), 6.95 (d, J=3.5 Hz, 1H), 4.82 (s, 1H), 4.33 (s, 2H), 4.17 (d, J=5.0 Hz, 2H), 3.05 (t, J=7.6 Hz, 2H), 1.88-1.75 (m, 2H), 1.43 (d, J=7.5 Hz, 2H), 1.19 (s, 6H), 0.95 (t, J=7.4 Hz, 3H).

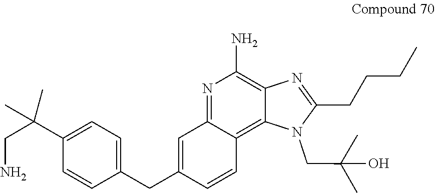 [SMILES: CCCCc1nc2c(N)nc3cc(Cc4ccc(C(C)(C)CN)cc4)ccc3c2n1CC(C)(C)O]

### 1-(4-amino-7-(4-(1-amino-2-methylpropan-2-yl)benzyl)-2-butyl-1H-imidazo[4,5-c]quinolin-1-yl)-2-methylpropan-2-ol

LCMS (ESI) calcd for C₂₉H₃₉N₅O [M+H]⁺ m/z=474.3, found 474.3.

¹H NMR (400 MHz, DMSO-d6, ppm) δ 13.79 (s, 1H), 8.91 (s, 1H), 8.42 (d, J=8.6 Hz, 1H), 7.67 (s, 3H), 7.59 (s, 1H), 7.37 (d, J=8.3 Hz, 3H), 7.28 (d, J=8.3 Hz, 2H), 4.82 (s, 1H), 4.11 (s, 2H), 3.04 (t, J=6.9 Hz, 4H), 1.86-1.74 (m, 3H), 1.42 (dd, J=14.9, 7.4 Hz, 2H), 1.32 (d, J=8.1 Hz, 6H), 1.18 (s, 6H), 0.94 (t, J=7.3 Hz, 3H).

Compound 71 was prepared by similar method as described in Example 10 using corresponding reagents.

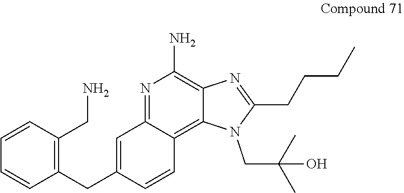 [SMILES: CCCCc1nc2c(N)nc3cc(Cc4ccccc4CN)ccc3c2n1CC(C)(C)O]

### 1-(4-amino-7-(2-(aminomethyl)benzyl)-2-butyl-1H-imidazo[4,5-c]quinolin-1-yl)-2-methylpropan-2-ol

LCMS (ESI) calcd for C₂₆H₃₃N₅O [M+H]⁺ m/z=432.3, found 432.2.

¹H NMR (400 MHz, DMSO-d6, ppm) δ 14.05 (s, 1H), 9.02 (s, 2H), 8.45 (d, J=8.7 Hz, 1H), 8.29 (s, 3H), 7.52-7.43 (m, 2H), 7.37 (dd, J=5.5, 3.6 Hz, 2H), 7.23 (dd, J=11.1, 7.4 Hz, 2H), 4.59 (d, J=115.7 Hz, 2H), 4.29 (s, 2H), 4.02 (q, J=5.4 Hz, 2H), 3.05 (t, J=7.6 Hz, 2H), 1.91-1.73 (m, 2H), 1.43 (dd, J=14.9, 7.4 Hz, 2H), 1.19 (s, 6H), 0.94 (t, J=7.3 Hz, 3H).

### Example 11

### Synthesis of Compound 22

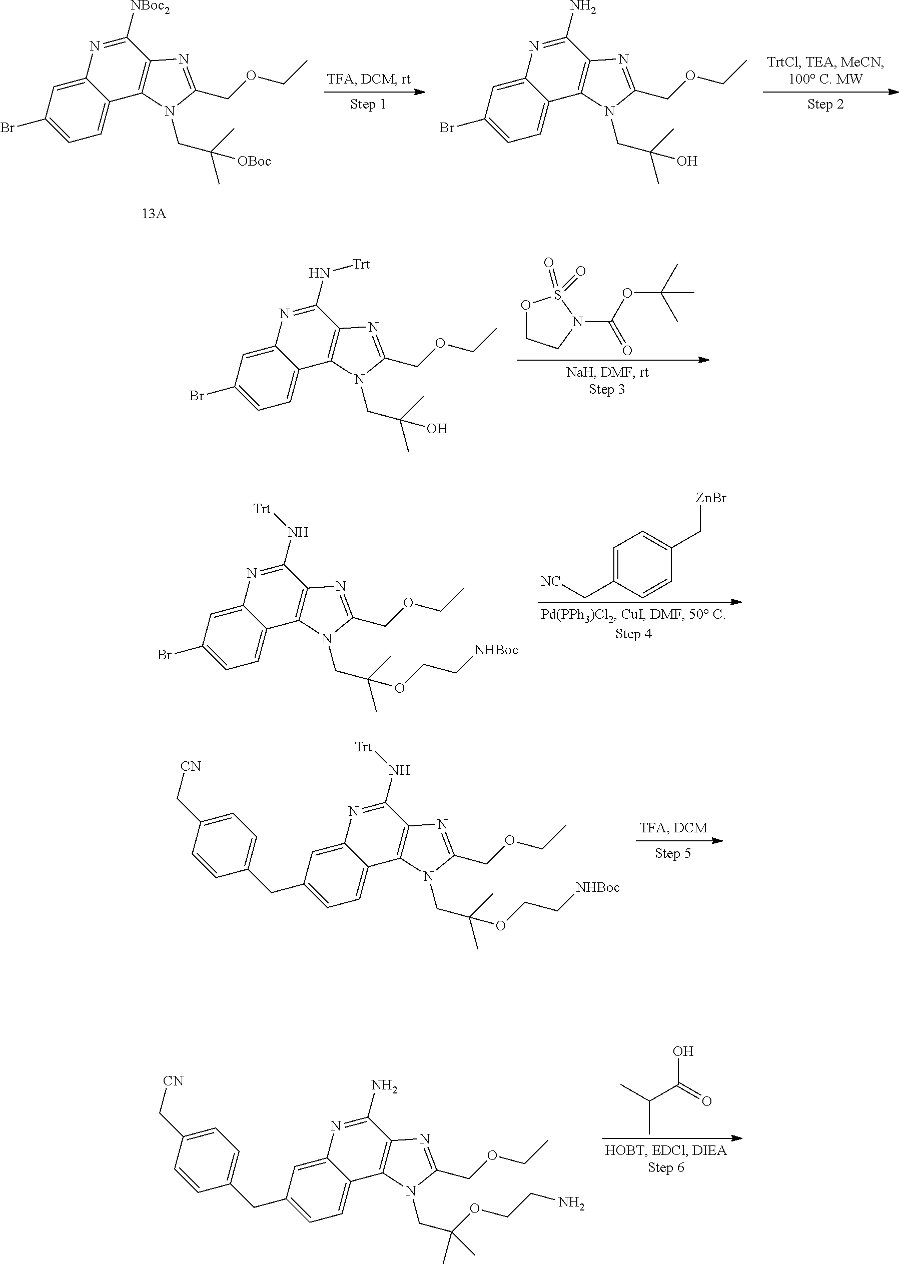

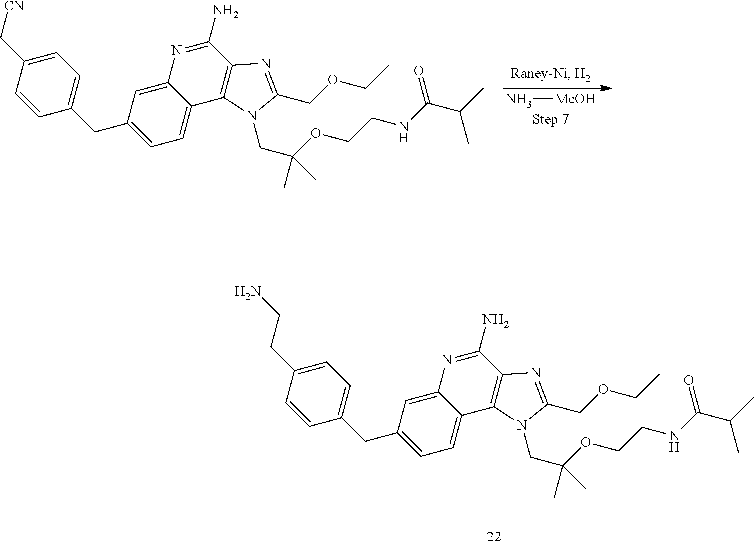

### Step 1: Preparation of 1-(4-amino-7-bromo-2-(ethoxymethyl)-1H-imidazo[4,5-c]quinolin-1-yl)-2-methylpropan-2-ol

To a solution of di-tert-butyl (7-bromo-1-(2-((tert-butoxycarbonyl)oxy)-2-methylpropyl)-2-(ethoxymethyl)-1H-imidazo[4,5-c]quinolin-4-yl)iminodicarbonate (5 g, 7.23 mmol) in DCM (30 mL) was added TFA (10 mL) dropwise at 0° C. The mixture was stirred at RT for 2 hours. The resulting mixture was concentrated in vacuo. The residue was adjusted to pH=8 with saturated sodium bicarbonate in aqueous solution. The resulting mixture was diluted with water (10 mL) and extracted with EA (20 mL×3). The combined organic phases were washed with brine, dried over Na₂SO₄, concentrated to give 1-(4-amino-7-bromo-2-(ethoxymethyl)-1H-imidazo[4,5-c]quinolin-1-yl)-2-methylpropan-2-ol (3.6 g).

LCMS (ESI) calcd for C₁₇H₂₁BrN₄O₂ [M+H]⁺ m/z=393.1, found 393.

### Step 2: Preparation of 1-(7-bromo-2-(ethoxymethyl)-4-(tritylamino)-1H-imidazo[4,5-c]quinolin-1-yl)-2-methylpropan-2-ol

A mixture of 1-(4-amino-7-bromo-2-(ethoxymethyl)-1H-imidazo[4,5-c]quinolin-1-yl)-2-methylpropan-2-ol (3.6 g, 9.15 mmol), TrtCl (3.8 g, 13.73 mmol) and Et₃N (2.3 g, 22.89 mmol) in MeCN (30 mL) was heated at 100° C. in a microwave reactor for 0.5 h under an atmosphere of N₂. LCMS showed the reaction was completed. The resulting mixture was cooled to 0′C with ice water. The precipitate was collected by filtration to afford 1-(7-bromo-2-(ethoxymethyl)-4-(tritylamino)-1H-imidazo[4,5-c]quinolin-1-yl)-2-methylpropan-2-ol (0.6 g, 10.3% yield) as white solid.

LCMS (ESI) calcd for C₃₆H₃₅BrN₄O₂[M+H]⁺ m/z=635.2, found 635.

### Step 3: Preparation of 2-((1-(7-bromo-2-(ethoxymethyl)-4-(tritylamino)-1H-imidazo[4,5-c]quinolin-1-yl)-2-methylpropan-2-yl)oxy)ethyl hydrogen sulfate

To a solution of NaH (190 mg, 4.75 mmol, 60%) in DMF (5 mL) was added 1-(7-bromo-2-(ethoxymethyl)-4-(tritylamino)-1H-imidazo[4,5-c]quinolin-1-yl)-2-methylpropan-2-ol (600 mg, 0.95 mmol) in DMF (8 mL). The mixture was stirred at RT for 1 h. Then 1,3,2-dioxathiolane 2,2-dioxide (350 mg, 2.84 mmol) was added to the mixture. The mixture was stirred at RT for 2 hours. LCMS showed the reaction was completed. The resulting mixture was diluted with water (30 mL) and extracted with EA (50 mL×3). The combine organic phases were washed with brine, dried over Na₂SO₄, concentrated to give the crude. The residue was purified by silica gel column chromatography (PE:EA=1:1) to afford 2-((1-(7-bromo-2-(ethoxymethyl)-4-(tritylamino)-1H-imidazo[4,5-c]quinolin-1-yl)-2-methylpropan-2-yl)oxy)ethyl hydrogen sulfate (500 mg, 69.74% yield) as yellow solid.

LCMS (ESI) calcd for C₃₈H₃₉BrN₄O₆S [M+H]⁺ m/z=759.2, found 759.

### Step 4: Preparation of di-tert-butyl (7-bromo-1-(2-(2-((tert-butoxycarbonyl)oxy)ethoxy)-2-methylpropyl)-2-(ethoxymethyl)-1H-imidazo[4,5-c]quinolin-4-yl)iminodicarbonate

To a solution of 2-((1-(4-amino-7-bromo-2-(ethoxymethyl)-1H-imidazo[4,5-c]quinolin-1-yl)-2-methylpropan-2-yl)oxy)ethan-1-ol (500 mg, 1.16 mmol), Pd(PPh₃)Cl₂ (81 mg, 0.12 mmol) and CuI (43 mg, 0.23 mmol) in DMF (10 mL) was added a solution of (4-(cyanomethyl)benzyl)zinc(II) bromide in DMF (15 mL). The mixture was stirred at 50′C for 1 h. LCMS showed the reaction was completed. The resulting mixture was diluted with water (60 mL) and extracted with EA (40 mL×3). The combine organic phases were washed with brine, dried over Na₂SO₄, concentrated to give the crude. The residue was purified by silica gel column chromatography (PE:EA=1:1) to afford tert-butyl (2-((1-(7-(4-(cyanomethyl)benzyl)-2-(ethoxymethyl)-4-(tritylamino)-1H-imidazo[4,5-c]quinolin-1-yl)-2-methylpropan-2-yl)oxy)ethyl)carbamate (550 mg, 57.35% yield) as yellow solid.

LCMS (ESI) calcd for C₅₂H₅₆N₆O₄ [M+H]⁺ m/z=829.4, found 829.

### Step 5: Preparation of 2-(4-((4-amino-1-(2-(2-aminoethoxy)-2-methylpropyl)-2-(ethoxymethyl)-1H-imidazo[4,5-c]quinolin-7-yl)methyl)phenyl)acetonitrile

To a solution of tert-butyl (2-((1-(7-(4-(cyanomethyl)benzyl)-2-(ethoxymethyl)-4-(tritylamino)-1H-imidazo[4,5-c]quinolin-1-yl)-2-methylpropan-2-yl)oxy)ethyl)carbamate (200 mg, 0.24 mmol) in DCM (3 mL) was added TFA (1 mL) dropwise at 0° C. The mixture was stirred at RT for 1 h. The resulting mixture was concentrated in vacuo. The residue was adjusted to pH=8 with saturated sodium bicarbonate in aqueous solution. The resulting mixture was diluted with water (10 mL) and extracted with EA (20 mL×3). The combine organic phases were washed with brine, dried over Na₂SO₄, concentrated to give the 2-(4-((4-amino-1-(2-(2-aminoethoxy)-2-methylpropyl)-2-(ethoxymethyl)-1H-imidazo[4,5-c]quinolin-7-yl)methyl) phenyl) acetonitrile (100 mg, 85.4% yield).

LCMS (ESI) calcd for C₂₈H₃₄N₆O₂ [M+H]⁺ m/z=487.3, found 487.

### Step 6: Preparation of N-(2-((1-(4-amino-7-(4-(cyanomethyl)benzyl)-2-(ethoxymethyl)-1H-imidazo[4,5-c]quinolin-1-yl)-2-methylpropan-2-yl)oxy)ethyl)isobutyramide

A solution of tert-butyl (2-((1-(7-(4-(cyanomethyl)benzyl)-2-(ethoxymethyl)-4-(tritylamino)-1H-imidazo[4,5-c]quinolin-1-yl)-2-methylpropan-2-yl)oxy)ethyl)carbamate (80 mg, 0.164 mmol), isobutyric acid (10 mg, 0.12 mmol), HOBT (67 mg, 0.49 mmol), EDCI (95 mg, 0.49 mmol) and DIEA (106 mg, 0.82 mmol) in DMF (3 mL) was stirred at rt for 2 hours. LCMS showed the reaction was completed. The resulting mixture was diluted with water (60 mL) and extracted with EA (40 mL×3). The combine organic phases were washed with brine, dried over Na₂SO₄, concentrated to give the crude. The residue was purified by silica gel column chromatography (PE:EA 2:1) to afford N-(2-((1-(4-amino-7-(4-(cyanomethyl)benzyl)-2-(ethoxymethyl)-1H-imidazo[4,5-c]quinolin-1-yl)-2-methylpropan-2-yl)oxy)ethyl)isobutyramide (30 mg, 32.78% yield) as yellow solid.

LCMS (ESI) calcd for C₃₂H₄₀N₆O₃ [M+H]⁺ m/z=557.3, found 557.

### Step 7: Preparation of N-(2-((1-(4-amino-7-(4-(2-aminoethyl)benzyl)-2-(ethoxymethyl)-1H-imidazo[4,5-c]quinolin-1-yl)-2-methylpropan-2-yl)oxy)ethyl)isobutyramide (Compound 22)

To a solution of N-(2-((1-(4-amino-7-(4-(cyanomethyl)benzyl)-2-(ethoxymethyl)-1H-imidazo[4,5-c]quinolin-1-yl)-2-methylpropan-2-yl)oxy)ethyl)isobutyramide (30 mg, 0.054 mmol) in NH₃-MeOH (3 mL) was added the right amount of Raney-Ni in MeOH. The mixture was stirred at RT for 2 hours under H₂. LCMS showed the reaction was completed. Then the mixture was filtered through celite and concentrated under vacuo to give the crude. The residue was purified by Prep-HPLC (Column: Gemini-C18,150×21.2 mm, 5 um; Mobile Phase: ACN-H₂O (0.05% NH₃), Gradient: 10-40) to give Compound 22 (4.25 mg, 14.07% yield) as white solid.

LCMS (ESI) calcd for C₃₂H₄₄N₆O₃ [M+H]⁺ m/z=561.4, found 561.

¹H NMR (400 MHz, DMSO-d₆) δ 8.18 (d, J=8.5 Hz, 1H), 7.38 (d, J=1.6 Hz, 1H), 7.29 (t, J=5.5 Hz, 1H), 7.15 (dd, J=25.6, 7.9 Hz, 4H), 7.07 (dd, J=8.4, 1.7 Hz, 1H), 6.49 (s, 2H), 4.71 (s, 2H), 4.00 (s, 2H), 3.49 (q, J=7.0 Hz, 2H), 3.21 (t, J=5.8 Hz, 2H), 3.00 (d, J=5.7 Hz, 2H), 2.77-2.60 (m, 4H), 2.22-2.18 (m, 1H), 1.28-1.03 (m, 9H), 0.92 (d, J=6.8 Hz, 6H).

Compound 21 was prepared by similar method as described in Example 11 using corresponding reagents.

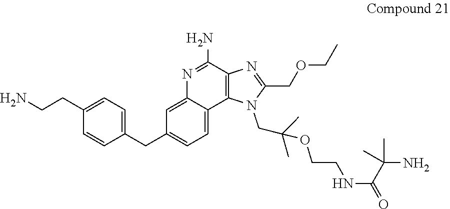 [SMILES: CCOCc1nc2c(N)nc3cc(Cc4ccc(CCN)cc4)ccc3c2n1CC(C)(C)OCCNC(=O)C(C)(C)N]

LCMS (ESI) calcd for C₃₂H₄₅N₇O₃ [M+H]⁺ m/z=576.4, found 576.

¹H NMR (400 MHz, DMSO) δ 8.31 (d, J=8.6 Hz, 1H), 7.49 (s, 1H), 7.20 (dd, J=19.4, 8.2 Hz, 5H), 4.09 (s, 2H), 3.63 (q, J=7.0 Hz, 2H), 3.37 (t, J=5.4 Hz, 2H), 3.10 (s, 2H), 2.92 (t, J=7.1 Hz, 2H), 2.77 (t, J=7.3 Hz, 2H), 1.36-1.19 (m, 11H), 1.14 (s, 6H).

### Example 12

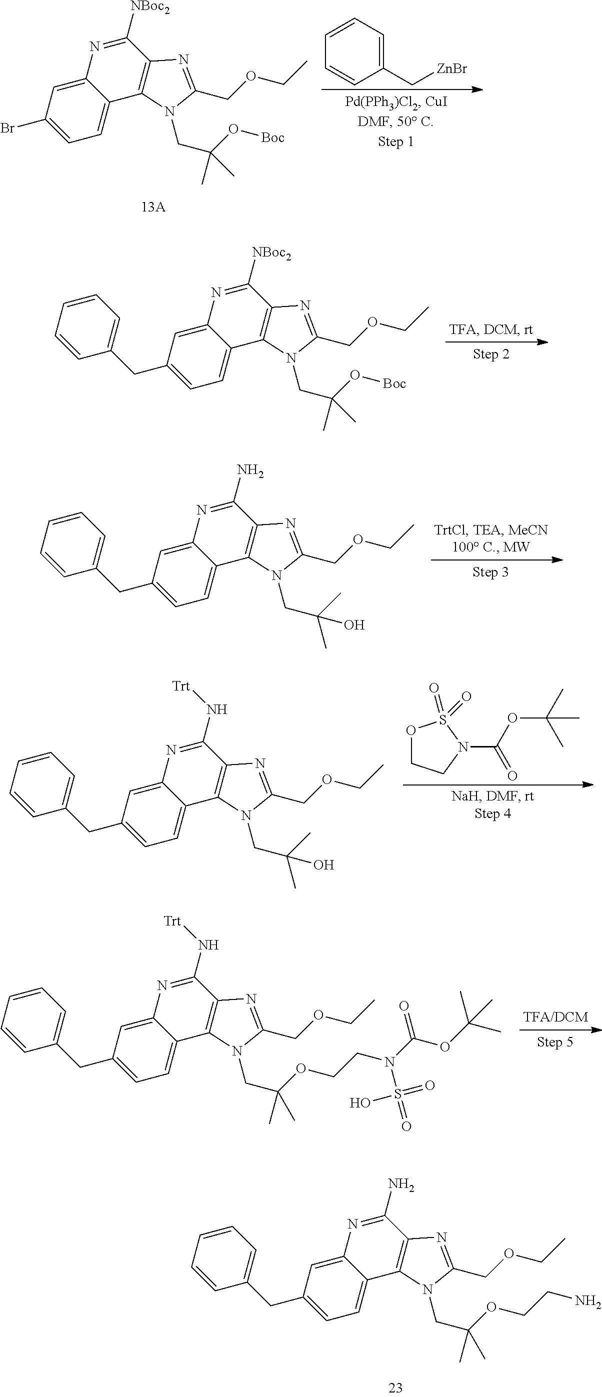

### Step 1: Preparation of di-tert-butyl (7-benzyl-1-(2-((tert-butoxycarbonyl) oxy)-2-methylpropyl)-2-(ethoxymethyl)-1H-imidazo[4,5-c]quinolin-4-yl) iminodicarbonate

To a solution of di-tert-butyl (7-bromo-1-(2-((tert-butoxycarbonyl) oxy)-2-methylpropyl)-2-(ethoxymethyl)-1H-imidazo[4,5-c]quinolin-4-yl) iminodicarbonate (500 mg, 0.72 mmol), Pd(PPh₃)Cl₂(152 mg, 0.2159 mmol) and CuI (27 mg, 0.1440 mmol) in DMF (3 mL) was added a solution of benzylzinc(II) bromide in DMF (10 mL). The mixture was stirred at 50′C for 0.5 h. LCMS showed the reaction was completed. The resulting mixture was diluted with water (60 mL) and extracted with EA (40 mL×3). The combine organic phases were washed with brine, dried over Na₂SO₄, concentrated to give the crude. The residue was purified by combi-flash (PE:EA 1:2) to afford di-tert-butyl (7-benzyl-1-(2-((tert-butoxycarbonyl) oxy)-2-methylpropyl)-2-(ethoxymethyl)-1H-imidazo[4,5-c]quinolin-4-yl) iminodicarbonate (500 mg, 98.42% yield) as yellow solid.

LCMS (ESI) calcd for C₃₉H₅₂N₄O₈ [M+H]⁺ m/z=705.4, found 705.

### Step 2: Preparation of 1-(4-amino-7-benzyl-2-(ethoxymethyl)-1H-imidazo[4,5-c]quinolin-1-yl)-2-methylpropan-2-ol

To a solution of di-tert-butyl (7-benzyl-1-(2-((tert-butoxycarbonyl) oxy)-2-methylpropyl)-2-(ethoxymethyl)-1H-imidazo[4,5-c]quinolin-4-yl) iminodicarbonate (500 mg, 0.71 mmol) in DCM (6 mL) was added TFA (2 mL) dropwise at 0° C. The mixture was stirred at RT for 2 hours. The resulting mixture was concentrated in vacuo. The residue was adjusted to pH=8 with saturated sodium bicarbonate in aqueous solution. The resulting mixture was extracted with EA (20 mL×3). The combine organic phases were washed with brine, dried over Na₂SO₄, concentrated to give the crude title compound.

LCMS (ESI) calcd for C₂₄H₂₈N₄O₂ [M+H]⁺ m/z=405.2, found 405.

### Step 3: Preparation of 1-(7-benzyl-2-(ethoxymethyl)-4-(tritylamino)-1H-imidazo[4,5-c]quinolin-1-yl)-2-methylpropan-2-ol

A mixture of 1-(4-amino-7-benzyl-2-(ethoxymethyl)-1H-imidazo[4,5-c]quinolin-1-yl)-2-methylpropan-2-ol (180 mg, 0.45 mmol), TrtCl (186 mg, 0.67 mmol) and Et₃N (112 mg, 1.11 mmol) in MeCN (4 mL) was heated at 100° C. in a microwave reactor for 0.5 h under an atmosphere of N₂. LCMS showed the reaction was completed. The resulting mixture was cooled to 0° C. with ice water. The precipitate was collected by filtration to afford 1-(7-benzyl-2-(ethoxymethyl)-4-(tritylamino)-1H-imidazo[4,5-c]quinolin-1-yl)-2-methylpropan-2-ol (210 mg, 72.97% yield) as white solid.

LCMS (ESI) calcd for C₄₃H₄₂N₄O₂ [M+H]⁺ m/z=467.3, found 467.

### Step 4: Preparation of (2-((1-(7-benzyl-2-(ethoxymethyl)-4-(tritylamino)-1H-imidazo[4,5-c]quinolin-1-yl)-2-methylpropan-2-yl) oxy) ethyl) (tert-butoxycarbonyl) sulfamic acid

To a solution of NaH (65 mg, 60% in oil) in DMF (3 mL) was added a solution of 1-(7-benzyl-2-(ethoxymethyl)-4-(tritylamino)-1H-imidazo[4,5-c]quinolin-1-yl)-2-methylpropan-2-ol (210 mg, 0.325 mmol) in DMF (2 mL) dropwise. The mixture was stirred at RT for 1 h. Then tert-butyl 1,2,3-oxathiazolidine-3-carboxylate 2,2-dioxide (217 mg, 0.97 mmol) was added to the mixture. The mixture was stirred at RT for 2 hours. LCMS showed the reaction was completed. The resulting mixture was quenched with water (30 mL) and extracted with EA (50 mL×3). The combine organic phases were washed with brine, dried over Na₂SO₄, concentrated to give the crude title compound. The residue was purified by combi-flash (PE:EA 1:2) to afford (2-((1-(7-benzyl-2-(ethoxymethyl)-4-(tritylamino)-1H-imidazo[4,5-c]quinolin-1-yl)-2-methylpropan-2-yl)oxy) ethyl) (tert-butoxycarbonyl)sulfamic acid (120 mg, 42.48% yield) as yellow solid.

LCMS (ESI) calcd for C₅₀H₅₅N₅O₇S [M+H]⁺ m/z=870.4, found 870.

### Step 5: Preparation of 1-(2-(2-aminoethoxy)-2-methylpropyl)-7-benzyl-2-(ethoxymethyl)-1H-imidazo[4,5-c]quinolin-4-amine (Compound 23)

To a solution of (2-((1-(7-benzyl-2-(ethoxymethyl)-4-(tritylamino)-1H-imidazo[4,5-c]quinolin-1-yl)-2-methylpropan-2-yl) oxy) ethyl) (tert-butoxycarbonyl) sulfamic acid (120 mg, 0.15 mmol) in DCM (4 mL) was added TFA (1.5 mL) dropwise at 0° C. The mixture was stirred at rt for 2 hours. LCMS showed the reaction was completed. The reaction was concentrated and purified by Prep-HPLC (Column: Gemini-C18, 150×21.2 mm, 5 um; Mobile Phase: ACN-H₂O (0.05% NH₃), Gradient: 10-40) to give Compound 23 (23 mg, 33.88% yield) as white solid.

LCMS (ESI) calcd for C₂₆H₃₃N₅O₂ [M+H]⁺ m/z=448.3, found 448.

¹H NMR (400 MHz, DMSO-d₆) δ 8.21 (d, J=8.5 Hz, 1H), 7.39 (d, J=1.7 Hz, 1H), 7.33-7.26 (m, 4H), 7.22-7.18 (m, 1H), 7.07 (dd, J=8.5, 1.8 Hz, 1H), 6.49 (s, 2H), 4.72 (s, 4H), 4.04 (s, 2H), 3.50 (q, J=7.0 Hz, 2H), 3.16 (t, J=5.7 Hz, 2H), 2.45 (t, J=5.7 Hz, 2H), 1.89 (s, 2H), 1.15 (s, 3H), 1.12 (t, J=7.0 Hz, 6H).

Compounds 28-31 were prepared by similar method as described in Example 11 using corresponding reagents.

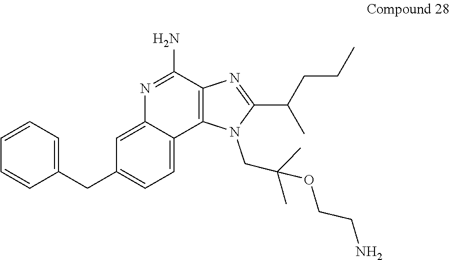 [SMILES: CCCC(C)c1nc2c(N)nc3cc(Cc4ccccc4)ccc3c2n1CC(C)(C)OCCN]

LCMS (ESI) calcd for C₂₈H₃₇N₅O [M+H]⁺ m/z=460.3, found 460.0.

¹H NMR (400 MHz, DMSO-d₆) δ 8.41-8.04 (m, 2H), 7.39 (s, 1H), 7.34-7.24 (m, 5H), 7.23-7.15 (m, 1H), 7.07 (dd, J=8.5, 1.7 Hz, 1H), 6.33 (s, 2H), 4.50 (s, 1H), 4.04 (s, 2H), 3.45 (d, J=6.4 Hz, 2H), 2.61 (s, 2H), 1.70-0.73 (m, 18H).

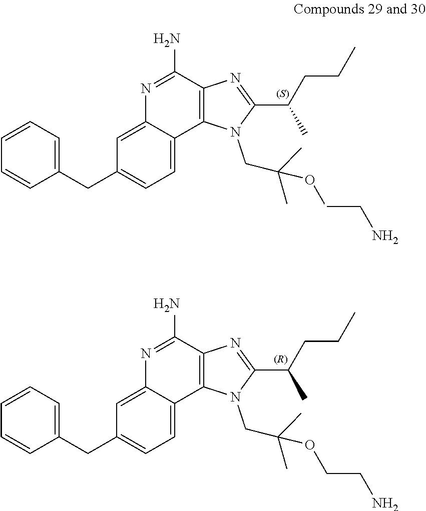

### (S)-1-(2-(2-aminoethoxy)-2-methylpropyl)-7-benzyl-2-(pentan-2-yl)-1H-imidazo[4,5-c]quinolin-4-amine (Compound 29) and (R)-1-(2-(2-aminoethoxy)-2-methylpropyl)-7-benzyl-2-(pentan-2-yl)-1H-imidazo[4,5-c]quinolin-4-amine (Compound 30)

The racemic 1-(2-(2-aminoethoxy)-2-methylpropyl)-7-benzyl-2-(pentan-2-yl)-1H-imidazo[4,5-c]quinolin-4-amine (Compound 28, 50 mg, 0.11 mmol) was separated by SFC (Apparatus: LC-30AD SFC; Column: DAICEL AS-H 4.6 mml. D.*250 mmL, 5 μm; Mobile phase: CO₂/MeOH (0.1% NH₃)=75/25; Flow rate: 2.0 ml/min; Wavelength: UV 214 nm & 254 nm; Temperature: 40° C.) to afford (S)-1-(2-(2-aminoethoxy)-2-methylpropyl)-7-benzyl-2-(pentan-2-yl)-1H-imidazo[4,5-c]quinolin-4-amine (Compound 29, 10 mg, 99% purity) and (R)-1-(2-(2-aminoethoxy)-2-methylpropyl)-7-benzyl-2-(pentan-2-yl)-1H-imidazo[4,5-c]quinolin-4-amine (Compound 30, 8 mg, 99% purity).

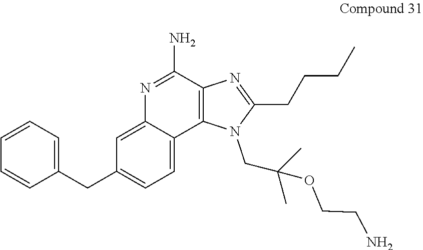 [SMILES: CCCCc1nc2c(N)nc3cc(Cc4ccccc4)ccc3c2n1CC(C)(C)OCCN]

LCMS (ESI) calcd for C₂₇H₃₅N₅O [M+H]⁺ m/z=446.3, found 446.

¹H NMR (400 MHz, DMSO-d₆) δ 8.26 (s, 1H), 8.19 (d, J=8.5 Hz, 1H), 7.40 (d, J=1.5 Hz, 1H), 7.33-7.25 (m, 4H), 7.24-7.16 (m, 1H), 7.08 (dd, J=8.5, 1.7 Hz, 1H), 6.49 (s, 2H), 4.62 (s, 2H), 4.04 (s, 2H), 3.40-3.28 (m, 3H), 3.02-2.89 (m, 2H), 2.66 (t, J=5.5 Hz, 2H), 1.81-1.74 (m, 2H), 1.41 (m, 2H), 1.16 (s, 6H), 0.94 (t, J=7.4 Hz, 3H).

Compound 44 were prepared by similar method as described in Example 12 using corresponding reagents.

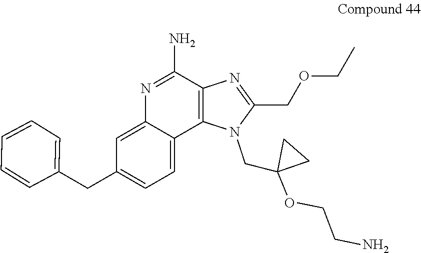 [SMILES: CCOCc1nc2c(N)nc3cc(Cc4ccccc4)ccc3c2n1CC1(OCCN)CC1]

LCMS (ESI) calcd for C₂₆H₃₂N₅O₂ [M+H]⁺ m/z=446.26, found 446.

¹H NMR (400 MHz, DMSO-d₆) δ 8.26 (s, 1H), 8.19 (d, J=8.5 Hz, 1H), 7.40 (d, J=1.5 Hz, 1H), 7.33-7.25 (m, 4H), 7.24-7.16 (m, 1H), 7.08 (dd, J=8.5, 1.7 Hz, 1H), 6.49 (s, 2H), 4.62 (s, 2H), 4.04 (s, 2H), 3.40-3.28 (m, 3H), 3.02-2.89 (m, 2H), 2.66 (t, J=5.5 Hz, 2H), 1.81-1.74 (m, 2H), 1.41 (m, 2H), 1.16 (s, 6H), 0.94 (t, J=7.4 Hz, 3H).

### Example 13

### Synthesis of Compound 24

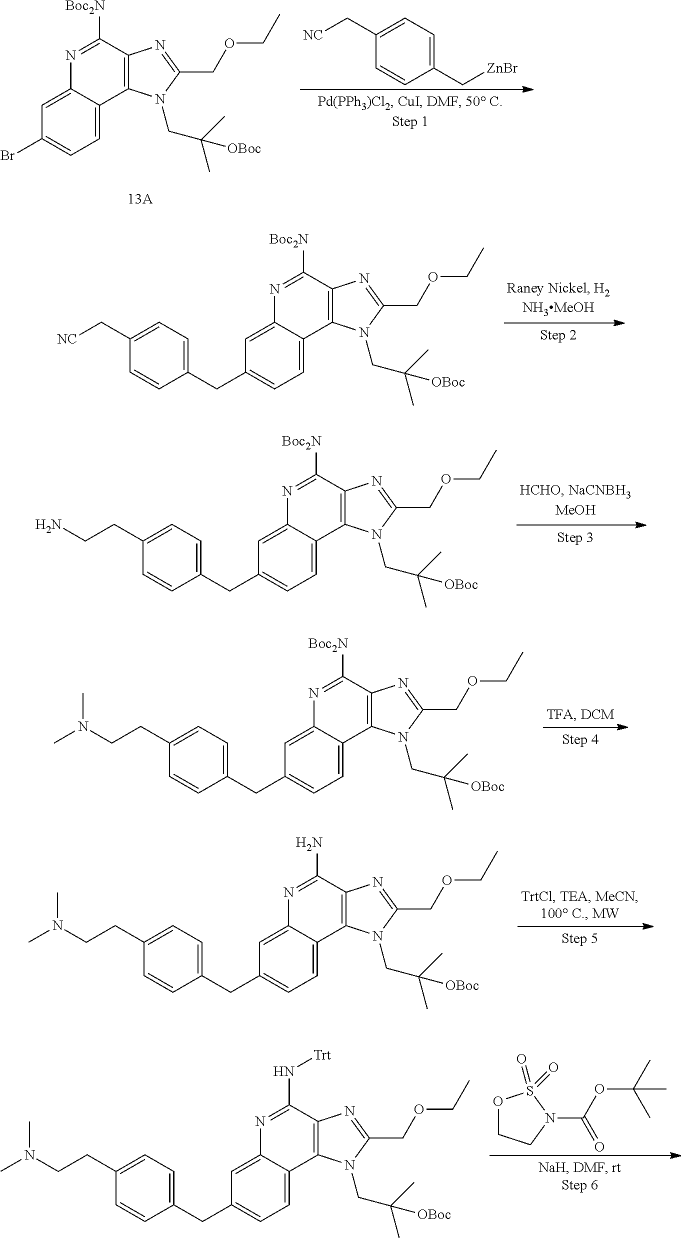

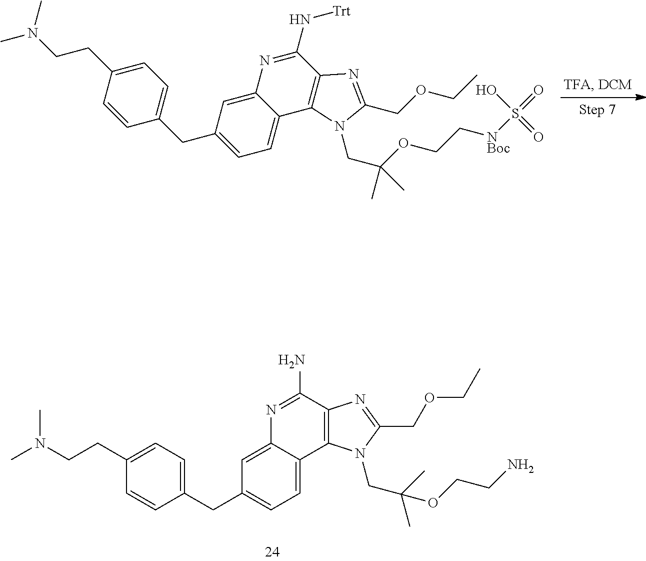

### Step 1: di-tert-butyl (1-(2-((tert-butoxycarbonyl)oxy)-2-methylpropyl)-7-(4-(cyanomethyl)benzyl)-2-(ethoxymethyl)-1H-imidazo[4,5-c]quinolin-4-yl)iminodicarbonate

To a solution of di-tert-butyl (7-bromo-1-(2-((tert-butoxycarbonyl)oxy)-2-methylpropyl)-2-(ethoxymethyl)-1H-imidazo[4,5-c]quinolin-4-yl)iminodicarbonate (400 mg, 0.57 mmol), PdCl₂(PPh₃)₂(81 mg, 0.12 mmol) and Copper(I) iodide (33 mg, 0.17 mmol) under N₂ was added (4-(cyanomethyl)benzyl)zinc(II) bromide (4 mL, 2M) dropwise. The reaction mixture was stirred at 50° C. for 1 h. LCMS showed the reaction was completed. The resulting mixture was diluted with water (50 mL) and extracted with EtOAc (50 mL×3). The combine organic phases were washed with brine, dried over Na₂SO₄, concentrated and purified by silica gel column chromatography (PE:EA 3:1) to give the title compound (400 mg, 83.9% yield) as a white solid.

LCMS (ESI) calcd for C₄₁H₅₃N₅O₈ [M+H]⁺ m/z=744.4, found 744.

### Step 2: di-tert-butyl (7-(4-(2-aminoethyl)benzyl)-1-(2-((tert-butoxycarbonyl)oxy)-2-methylpropyl)-2-(ethoxymethyl)-1H-imidazo[4,5-c]quinolin-4-yl)iminodicarbonate

To a solution of di-tert-butyl (1-(2-((tert-butoxycarbonyl)oxy)-2-methylpropyl)-7-(4-(cyanomethyl)benzyl)-2-(ethoxymethyl)-1H-imidazo[4,5-c]quinolin-4-yl)iminodicarbonate (400 mg, 0.54 mmol) in NH₃-MeOH (10 mL) was added a solution of Raney-Ni in MeOH. The reaction mixture was stirred at RT for 1 h. LCMS showed the reaction was completed. The resulting mixture was filtered through celite and the filtrate was concentrated under vacuum. The residue was purified by silica gel column chromatography (PE:EA 3:1) to give the title compound (350 mg, 87% yield) as a white solid.

LCMS (ESI) calcd for C₄₁H₅₇N₅O₈ [M+H]⁺ m/z=748.4, found 748.

### Step 3: di-tert-butyl (1-(2-((tert-butoxycarbonyl)oxy)-2-methylpropyl)-7-(4-(2-(dimethylamino)ethyl)benzyl)-2-(ethoxymethyl)-1H-imidazo[4,5-c]quinolin-4-yl)iminodicarbonate

To a solution of di-tert-butyl (7-(4-(2-aminoethyl)benzyl)-1-(2-((tert-butoxycarbonyl)oxy)-2-methylpropyl)-2-(ethoxymethyl)-1H-imidazo[4,5-c]quinolin-4-yl)iminodicarbonate (350 mg, 0.4679 mmol) and formaldehyde (3 mL, 33%) in MeOH (10 mL) stirred at 0° C. was added sodium cyanoborohydride (58 mg, 0.93 mmol). The reaction mixture was stirred at RT for 1 h. The mixture was concentrated to give the crude. The residue was purified by flash chromatography (MeOH:DCM 20:1) to give the title compound (300 mg, 82.6% yield) as white oil.

LCMS (ESI) calcd for C₄₃H₆₁N₅O₈ [M+H]⁺ m/z=776.4, found 776.

### Step 4: 1-(4-amino-7-(4-(2-(dimethylamino)ethyl)benzyl)-2-(ethoxymethyl)-1H-imidazo[4,5-c]quinolin-1-yl)-2-methylpropan-2-ol

To a solution of di-tert-butyl (1-(2-((tert-butoxycarbonyl)oxy)-2-methylpropyl)-7-(4-(2-(dimethylamino)ethyl)benzyl)-2-(ethoxymethyl)-1H-imidazo[4,5-c]quinolin-4-yl)iminodicarbonate (300 mg, 0.38 mmol) in DCM (6 mL) was added TFA (2 mL). The mixture was stirred at RT for 4 hours. LCMS showed the reaction was completed. The resulting mixture was adjusted to Ph=8 with aqueous NaHCO₃ and extracted with EA (50 mL×3). The combined organic phases were washed with water and brine, dried over sodium sulfate, concentrated under vacuum to give the crude. The crude was purified by silica gel column chromatography (PE:EA 1:1) to give the title compound (160 mg, 86.9% yield) as a white solid.

LCMS (ESI) calcd for C₂₈H₃₇N₅O₂ [M+H]⁺ m/z=476.2, found 476.

### Step 5: 1-(7-(4-(2-(dimethylamino)ethyl)benzyl)-2-(ethoxymethyl)-4-(tritylamino)-1H-imidazo[4,5-c]quinolin-1-yl)-2-methylpropan-2-ol

A solution of 1-(4-amino-7-(4-(2-(dimethylamino)ethyl)benzyl)-2-(ethoxymethyl)-1H-imidazo[4,5-c]quinolin-1-yl)-2-methylpropan-2-ol (160 mg, 0.34 mmol), triphenylmethyl chloride (113 mg, 0.4036 mmol) and TEA (86 mg, 0.841 mmol) in MeCN (6 mL) was stirred at 100° C. in a microwave reactor for 30 minutes. The mixture was concentrated and purified by combi-flash (PE:EA 2:1) to give the title compound (120 mg, 44.7% yield) as a white solid.

LCMS (ESI) calcd for C₄₇H₅₁N₅O₂ [M+H]⁺ m/z=718.4, found 718.

### Step 6: (tert-butoxycarbonyl)(2-((1-(7-(4-(2-(dimethylamino)ethyl)benzyl)-2-(ethoxymethyl)-4-(tritylamino)-1H-imidazo[4,5-c]quinolin-1-yl)-2-methylpropan-2-yl)oxy)ethyl)sulfamic acid

1-(7-(4-(2-(dimethylamino)ethyl)benzyl)-2-(ethoxymethyl)-4-(tritylamino)-1H-imidazo[4,5-c]quinolin-1-yl)-2-methylpropan-2-ol (120 mg, 0.1671 mmol) was added to a solution of NaH (6 mg, 0.25 mmol, 60%) in DMF (5 mL) at 0° C. Then the reaction mixture was warmed to RT and stirred for 30 minutes. Then tert-butyl 1,2,3-oxathiazolidine-3-carboxylate 2,2-dioxide (56 mg, 0.25 mmol) was added to the mixture at 0° C. The temperature was warmed to room temperature and stirred 16 hrs. The resulting mixture was diluted with water (30 mL) and extracted with EtOAc (30 mL×3). The combine organic phases were washed with brine, dried over Na₂SO₄, concentrated to give the crude and purified by silica gel column chromatography (PE:EA 1:1) to give the title compound (45 mg, 28.1% yield) as a white solid.

LCMS (ESI) calcd for C₅₄H₆₄N₆O₇S [M+H]⁺ m/z=941.4, found 941.

### Step 7: 1-(2-(2-aminoethoxy)-2-methylpropyl)-7-(4-(2-(dimethylamino)ethyl)benzyl)-2-(ethoxymethyl)-1H-imidazo[4,5-c]quinolin-4-amine (Compound 24)

To a solution of (tert-butoxycarbonyl)(2-((1-(7-(4-(2-(dimethylamino)ethyl)benzyl)-2-(ethoxymethyl)-4-(tritylamino)-1H-imidazo[4,5-c]quinolin-1-yl)-2-methylpropan-2-yl)oxy)ethyl)sulfamic acid (45 mg, 0.05 mmol) in DCM (3 mL) was added TFA (1 mL) dropwise at room temperature. The reaction mixture was stirred at room temperature for 3 hours. The solution was removed under reduced pressure to afford the crude product. The residue was purified by Prep-HPLC (column: Gemini-C18 150×21.2 mm, 5 um; mobile phase: ACN-H₂O (0.1% FA); gradient: 5-10-30) to afford Compound 24 (5.79 mg, 21.2% yield) as white solid.

LCMS (ESI) calcd for C₃₀H₄₂N₆O₂ [M+H]⁺ m/z=519.3, found 519.

¹H-NMR (400 MHz, MeOD) δ 8.42 (s, 1H), 8.38 (d, J=8.6 Hz, 1H), 7.59 (s, 1H), 7.40 (d, J=8.6 Hz, 1H), 7.32 (s, 4H), 4.96 (s, 2H), 4.81 (s, 4H), 4.16 (s, 2H), 3.73-3.49 (m, 6H), 3.31-3.26 (m, 2H), 3.24 (s, 6H), 3.14 (m, 2H), 1.27 (m, 9H).

### Example 14

### Synthesis of Compound 25

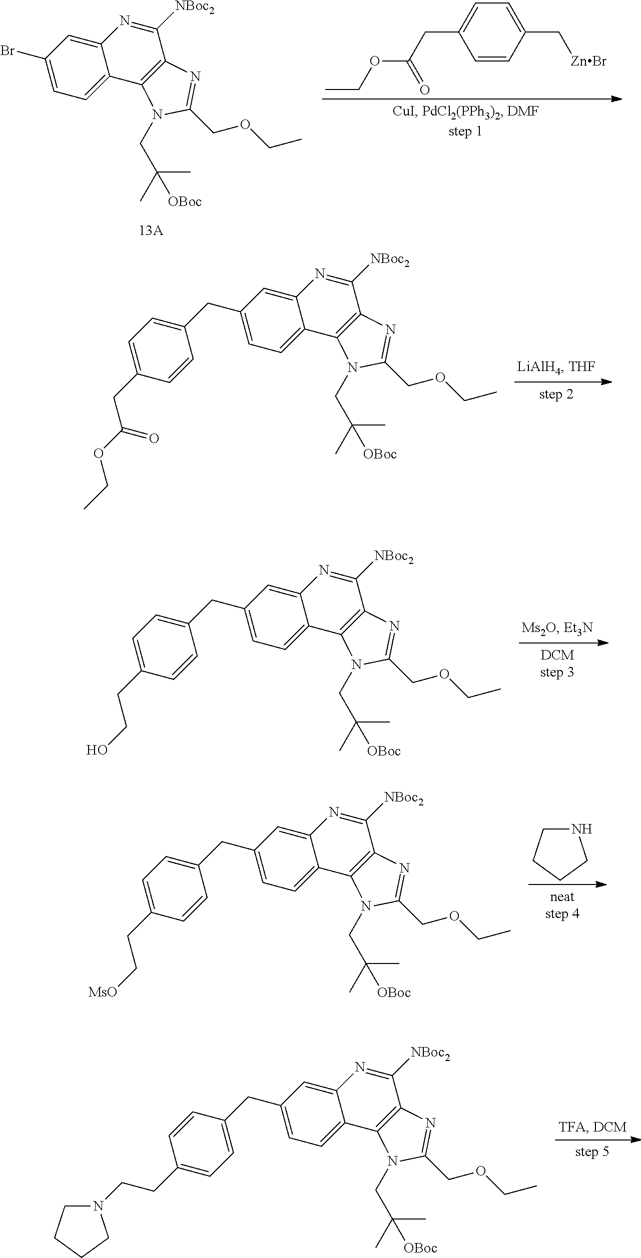

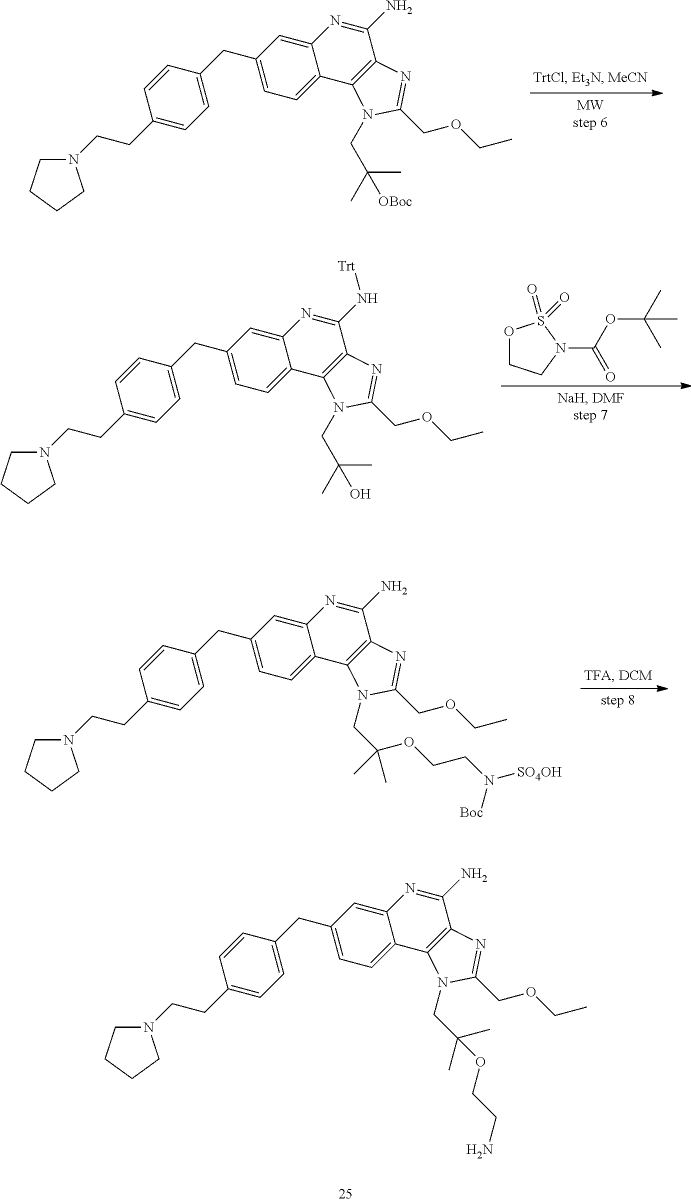

### Step 1: Preparation of ethyl 2-(4-((4-(bis(tert-butoxycarbonyl)amino)-1-(2-((tert-butoxycarbonyl)oxy)-2-methylpropyl)-2-(ethoxymethyl)-1H-imidazo[4,5-c]quinolin-7-yl)methyl)phenyl)acetate

To a stirred solution of di-tert-butyl (7-bromo-1-(2-((tert-butoxycarbonyl)oxy)-2-methylpropyl)-2-(ethoxymethyl)-1H-imidazo[4,5-c]quinolin-4-yl)iminodicarbonate (1.5 g, 2.2 mmol), CuI (0.08 g, 0.4 mmol) and PdCl₂(PPh₃)₂ (0.15 g, 0.2 mmol) in DMF (8 mL) was added a solution of bromo({[4-(2-ethoxy-2-oxoethyl)phenyl]methyl})zinc (1.06 g, 3.3 mmol) in DMF (8 mL) under an atmosphere of N₂. The reaction mixture was heated at 50° C. for 2 hours. LCMS showed the reaction was completed. The resulting mixture was diluted with water (60 mL) and extracted with EtOAc (60 mL×3). The organic phases were washed with brine, dried over Na₂SO₄, concentrated purified by column chromatography on silica gel (PE/EtOAc=1/1) to give ethyl 2-(4-((4-(bis(tert-butoxycarbonyl)amino)-1-(2-((tert-butoxycarbonyl)oxy)-2-methylpropyl)-2-(ethoxymethyl)-1H-imidazo[4,5-c]quinolin-7-yl)methyl)phenyl)acetate (1.26 g, 72.4% yield) as yellow solid.

LCMS (ESI) calcd for C₄₃H₅₈N₄O₁₀ [M+H]⁺ m/z=791.4, found 791.0.

### Step 2: Preparation of di-tert-butyl (1-(2-((tert-butoxycarbonyl)oxy)-2-methylpropyl)-2-(ethoxymethyl)-7-(4-(2-hydroxyethyl)benzyl)-1H-imidazo[4,5-c]quinolin-4-yl)iminodicarbonate

To a suspension of LiAlH₄ (90 mg, 2.4 mmol) in THF (5 mL) was added a solution of ethyl 2-(4-((4-(bis(tert-butoxycarbonyl)amino)-1-(2-((tert-butoxycarbonyl)oxy)-2-methylpropyl)-2-(ethoxymethyl)-1H-imidazo[4,5-c]quinolin-7-yl)methyl)phenyl)acetate (1.26 g, 1.6 mmol) in THF (10 mL) dropwise at 0° C. The reaction mixture was stirred at room temperature for 2 hours. LCMS showed the reaction was completed. The mixture was poured into ice-water (15 mL) and extracted with EA (20 mL×3). The combined organic phases were washed brine, dried over Na₂SO₄, concentrated and purified by column chromatography on silica gel (PE/EtOAc=1/2) to give di-tert-butyl (1-(2-((tert-butoxycarbonyl)oxy)-2-methylpropyl)-2-(ethoxymethyl)-7-(4-(2-hydroxyethyl)benzyl)-1H-imidazo[4,5-c]quinolin-4-yl)iminodicarbonate (600 mg, 50.1% yield) as a yellow solid.

LCMS (ESI) calcd for C₄₁H₅₆N₄O₉[M+H]⁺ m/z=749.4, found 749.0.

### Step 3: Preparation of 4-((4-(bis(tert-butoxycarbonyl)amino)-1-(2-((tert-butoxycarbonyl)oxy)-2-methylpropyl)-2-(ethoxymethyl)-1H-imidazo[4,5-c]quinolin-7-yl)methyl)phenethyl methanesulfonate

To a stirred solution of di-tert-butyl (1-(2-((tert-butoxycarbonyl)oxy)-2-methylpropyl)-2-(ethoxymethyl)-7-(4-(2-hydroxyethyl)benzyl)-1H-imidazo[4,5-c]quinolin-4-yl)iminodicarbonate (600 mg, 0.8 mmol), Et₃N (161.6 mg, 1.6 mmol) in DCM (5 mL) was added a solution of methanesulfonic anhydride (417.6 mg, 2.4 mmol) in DCM (5 mL) dropwise at 0° C. The reaction mixture was stirred at room temperature for 2 hours. LCMS showed the reaction was completed. The resulting mixture was diluted with water (20 mL) and extracted with DCM (20 mL×3). The organic phases were dried over Na₂SO₄, concentrated to give the crude 4-((4-(bis(tert-butoxycarbonyl)amino)-1-(2-((tert-butoxycarbonyl)oxy)-2-methylpropyl)-2-(ethoxymethyl)-1H-imidazo[4,5-c]quinolin-7-yl)methyl)phenethyl methanesulfonate (600 mg) which was used in next reaction without further purification.

LCMS (ESI) calcd for C₄₂H₅₈N₄O₁₁S [M+H]⁺ m/z=827.4, found 827.0.

### Step 4: Preparation of di-tert-butyl (1-(2-((tert-butoxycarbonyl)oxy)-2-methylpropyl)-2-(ethoxymethyl)-7-(4-(2-(pyrrolidin-1-yl)ethyl)benzyl)-1H-imidazo[4,5-c]quinolin-4-yl)iminodicarbonate

A mixture of 4-((4-(bis(tert-butoxycarbonyl)amino)-1-(2-((tert-butoxycarbonyl)oxy)-2-methylpropyl)-2-(ethoxymethyl)-1H-imidazo[4,5-c]quinolin-7-yl)methyl)phenethyl methanesulfonate (500 mg, 0.60 mmol) in pyrrolidine (5 mL) was stirred at room temperature for 8 hours. The resulting mixture was diluted with water (20 mL) and extracted with DCM (20 mL×3). The organic phases were dried over Na₂SO₄, concentrated and purified by column chromatography on silica gel (DCM/MeOH=50/1) to give di-tert-butyl (1-(2-((tert-butoxycarbonyl)oxy)-2-methylpropyl)-2-(ethoxymethyl)-7-(4-(2-(pyrrolidin-1-yl)ethyl)benzyl)-1H-imidazo[4,5-c]quinolin-4-yl)iminodicarbonate (400 mg, 83.1% yield) as a yellow solid.

LCMS (ESI) calcd for C₄₅H₆₃N₅O₈ [M+H]⁺ m/z=802.5, found 802.0.

### Step 5: Preparation of 1-(4-amino-2-(ethoxymethyl)-7-(4-(2-(pyrrolidin-1-yl)ethyl)benzyl)-1H-imidazo[4,5-c]quinolin-1-yl)-2-methylpropan-2-ol

To a solution of di-tert-butyl (1-(2-((tert-butoxycarbonyl)oxy)-2-methylpropyl)-2-(ethoxymethyl)-7-(4-(2-(pyrrolidin-1-yl)ethyl)benzyl)-1H-imidazo[4,5-c]quinolin-4-yl)iminodicarbonate (400 mg, 0.5 mmol) in DCM (4 mL) was added TFA (2 mL) dropwise at 0° C. The mixture was stirred at room temperature for 2 hours. The resulting mixture was concentrated in vacuo. The residue was diluted with EtOAc (20 mL) and adjusted to pH 7-8 with saturated aqueous NaHCO₃ solution. The organic phases were washed with brine, dried over Na₂SO₄, concentrated to give the 1-(4-amino-2-(ethoxymethyl)-7-(4-(2-(pyrrolidin-1-yl)ethyl)benzyl)-1H-imidazo[4,5-c]quinolin-1-yl)-2-methylpropan-2-ol (200 mg) as colorless oil which was used in next reaction without further purification.

LCMS (ESI) calcd for C₃₀H₃₉N₅O₂ [M+H]⁺ m/z=501.3, found 501.0.

### Step 6: Preparation of 1-(2-(ethoxymethyl)-7-(4-(2-(pyrrolidin-1-yl)ethyl)benzyl)-4-(tritylamino)-1H-imidazo[4,5-c]quinolin-1-yl)-2-methylpropan-2-ol

A mixture of 1-(4-amino-2-(ethoxymethyl)-7-(4-(2-(pyrrolidin-1-yl)ethyl)benzyl)-1H-imidazo[4,5-c]quinolin-1-yl)-2-methylpropan-2-ol (200 mg, 0.4 mmol), TrtCl (166.8 mg, 0.6 mmol) and Et₃N (101 mg, 1 mmol) in MeCN (2 mL) was heated at 100° C. for 30 minutes in a microwave reactor. LCMS showed the reaction was completed. The resulting mixture was concentrated and purified by silica gel column chromatography (DCM/MeOH=100/1) to give 1-(2-(ethoxymethyl)-7-(4-(2-(pyrrolidin-1-yl)ethyl)benzyl)-4-(tritylamino)-1H-imidazo[4,5-c]quinolin-1-yl)-2-methylpropan-2-ol (100 mg, 33.6% yield) as a yellow solid.

LCMS (ESI) calcd for C₄₉H₅₃N₅O₂ [M+H]⁺ m/z=744.4, found 744.0.

### Step 7: Preparation of tert-butyl (2-((1-(4-amino-2-(ethoxymethyl)-7-(4-(2-(pyrrolidin-1-yl)ethyl)benzyl)-1H-imidazo[4,5-c]quinolin-1-yl)-2-methylpropan-2-yl)oxy)ethyl)(trioxidaneylsulfonyl)carbamate

To a suspension of NaH (9.68 mg, 0.40 mmol) in DMF (3 mL) stirred at 0° C. was added a solution of 1-(2-(ethoxymethyl)-7-(4-(2-(pyrrolidin-1-yl)ethyl)benzyl)-4-(tritylamino)-1H-imidazo[4,5-c]quinolin-1-yl)-2-methylpropan-2-ol in DMF (3 mL) dropwise. The reaction mixture was stirred at room temperature for 1 hours. Then tert-butyl 1,2,3-oxathiazolidine-3-carboxylate 2,2-dioxide (90.01 mg, 0.4 mmol) was added at 0° C. The reaction mixture was stirred at 25° C. for 12 hours. LCMS showed the reaction was completed. The resulting mixture was diluted with water (15 mL) and extracted with EtOAc (15 mL×3). The organic phases were washed with brine, dried over Na₂SO₄, concentrated and purified by column chromatography on silica gel (DCM/MeOH=50/1) to give the tert-butyl (2-((1-(4-amino-2-(ethoxymethyl)-7-(4-(2-(pyrrolidin-1-yl)ethyl)benzyl)-1H-imidazo[4,5-c]quinolin-1-yl)-2-methylpropan-2-yl)oxy)ethyl)(trioxidaneylsulfonyl) carbamate (50 mg, 50.8%) as a white solid.

LCMS (ESI) calcd for C₃₇H₅₂N₆O₉S [M+H]⁺ m/z=757.4, found 757.0.

### Step 8: Preparation of 1-(2-(2-aminoethoxy)-2-methylpropyl)-2-(ethoxymethyl)-7-(4-(2-(pyrrolidin-1-yl)ethyl)benzyl)-1H-imidazo[4,5-c]quinolin-4-amine (Compound 25)

To a solution of tert-butyl (2-((1-(4-amino-2-(ethoxymethyl)-7-(4-(2-(pyrrolidin-1-yl)ethyl)benzyl)-1H-imidazo[4,5-c]quinolin-1-yl)-2-methylpropan-2-yl)oxy)ethyl)(trioxidaneylsulfonyl)carbamate (50 mg, 0.05 mmol) in DCM (1 mL) was added TFA (0.5 mL) dropwise at 0° C. The mixture was stirred at room temperature for 2 hours. LCMS showed the reaction was completed. The reaction mixture was concentrated and purified by prep-HPLC (Column: Gemini-C₁₈, 150×21.2 mm, 5 um; Mobile Phase: ACN-H₂O (0.1% FA), Gradient: 10%-40%) to give 1-(2-(2-aminoethoxy)-2-methylpropyl)-2-(ethoxymethyl)-7-(4-(2-(pyrrolidin-1-yl)ethyl)benzyl)-1H-imidazo[4,5-c]quinolin-4-amine (Compound 25, 4 mg, 14.7% yield) as white solid.

LCMS (ESI) calcd for C₃₂H₄₄N₆O₂ [M+H]⁺ m/z=545.4, found 545.0.

¹H NMR (400 MHz, CD₃OD, ppm) δ 8.40 (d, J=8.6 Hz, 1H), 7.57 (s, 1H), 7.42 (d, J=8.6 Hz, 1H), 7.32 (q, J=8.4 Hz, 4H), 5.01 (s, 2H), 4.82 (s, 2H), 4.17 (s, 2H), 3.85-3.54 (m, 10H), 3.37 (s, 2H), 3.15 (dd, J=10.1, 6.8 Hz, 2H), 2.26 (s, 4H), 1.30 (s, 6H), 1.26 (t, J=7.0 Hz, 3H).

### Example 15

### Synthesis of Compound 26

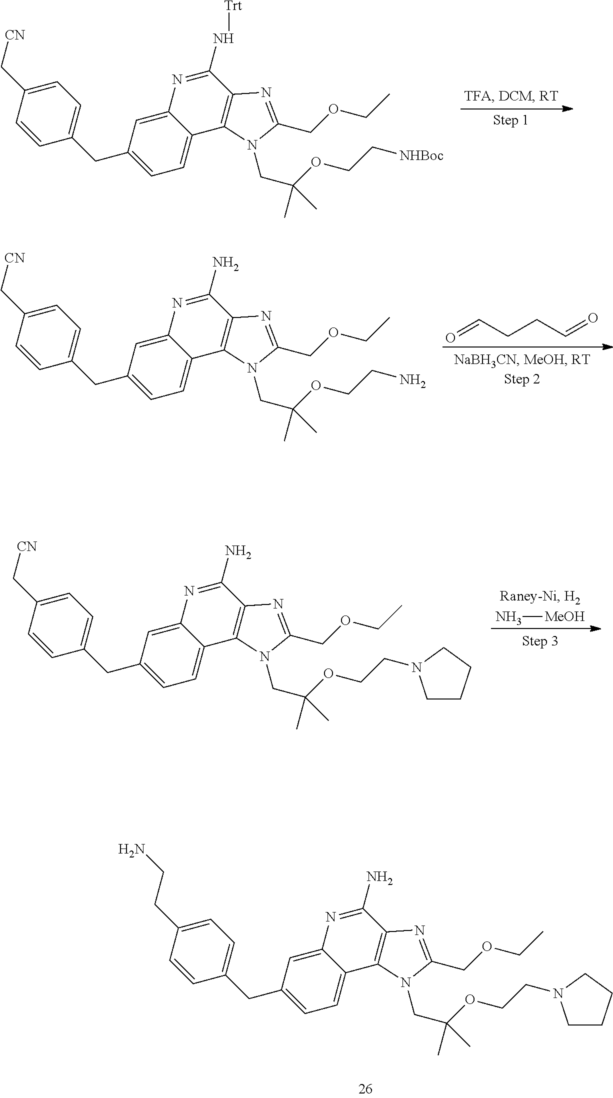

### Step 1: Preparation of 2-(4-((4-amino-1-(2-(2-aminoethoxy)-2-methylpropyl)-2-(ethoxymethyl)-1H-imidazo[4,5-c]quinolin-7-yl)methyl)phenyl)acetonitrile

To a solution of tert-butyl (2-((1-(7-(4-(cyanomethyl)benzyl)-2-(ethoxymethyl)-4-(tritylamino)-1H-imidazo[4,5-c]quinolin-1-yl)-2-methylpropan-2-yl)oxy)ethyl)carbamate (200 mg, 0.24 mmol) in DCM (6 mL) was added TFA (2 mL) dropwise at 0′C. The mixture was stirred at RT for 2 hours. LCMS showed the reaction was completed. The mixture was adjusted to pH=8 with NaHCO₃. The solution was extracted with DCM (20 mL×3). The organic phases were washed with brine, dried over Na₂SO₄, concentrated and purified by column chromatography on silica gel (PE/EA=1/2) to give 2-(4-((4-amino-1-(2-(2-aminoethoxy)-2-methylpropyl)-2-(ethoxymethyl)-1H-imidazo[4,5-c]quinolin-7-yl)methyl)phenyl)acetonitrile (80 mg, 85.3% yield).

LCMS (ESI) calcd for C₂₈H₃₄N₆O₂ [M+H]⁺ m/z=487.3, found 487.

### Step 2: Preparation of 2-(4-((4-amino-2-(ethoxymethyl)-1-(2-methyl-2-(2-(pyrrolidin-1-yl)ethoxy)propyl)-1H-imidazo[4,5-c]quinolin-7-yl)methyl)phenyl)acetonitrile

To a solution of 2-(4-((4-amino-1-(2-(2-aminoethoxy)-2-methylpropyl)-2-(ethoxymethyl)-1H-imidazo[4,5-c]quinolin-7-yl)methyl)phenyl)acetonitrile (100 mg, 0.20 mmol), succinaldehyde (35.35 mg, 0.4 mmol) in MeOH (10 ml) was added NaBH₃CN (25.83 mg, 0.41 mmol). The reaction mixture was stirred at RT for 1 hour. LCMS showed a peak of TM. It was concentrated and purified by column chromatography on silica gel (DCM:MeOH 20:1) to give the 2-(4-((4-amino-2-(ethoxymethyl)-1-(2-methyl-2-(2-(pyrrolidin-1-yl)ethoxy)propyl)-1H-imidazo[4,5-c]quinolin-7-yl)methyl)phenyl)acetonitrile (35 mg, 31.4% yield).

LCMS (ESI) calcd for C₃₂H₄₀N₆O₂ [M+H]⁺ m/z=541.5, found 541.

### Step 3: Preparation of 7-(4-(2-aminoethyl)benzyl)-2-(ethoxymethyl)-1-(2-methyl-2-(2-(pyrrolidin-1-yl)ethoxy)propyl)-1H-imidazo[4,5-c]quinolin-4-amine (Compound 26)

A solution of 2-(4-((4-amino-2-(ethoxymethyl)-1-(2-methyl-2-(2-(pyrrolidin-1-yl)ethoxy)propyl)-1H-imidazo[4,5-c]quinolin-7-yl)methyl)phenyl)acetonitrile (50 mg, 0.09 mmol) in NH₃-MeOH (5 ml) was added amount of Raney nickel washed with MeOH. The mixture was stirred at RT for 3 hours. It was filtrated and the filtrate was concentrated to give the crude product. The residue was purified by combi-flash (DCM:MeOH 25:1) to give the product as white solid.

LCMS (ESI) calcd for C₃₂H₄₄N₆O₂ [M+H]⁺ m/z=545.5, found 545.

### Example 16

### Synthesis of Compounds 27 and 73

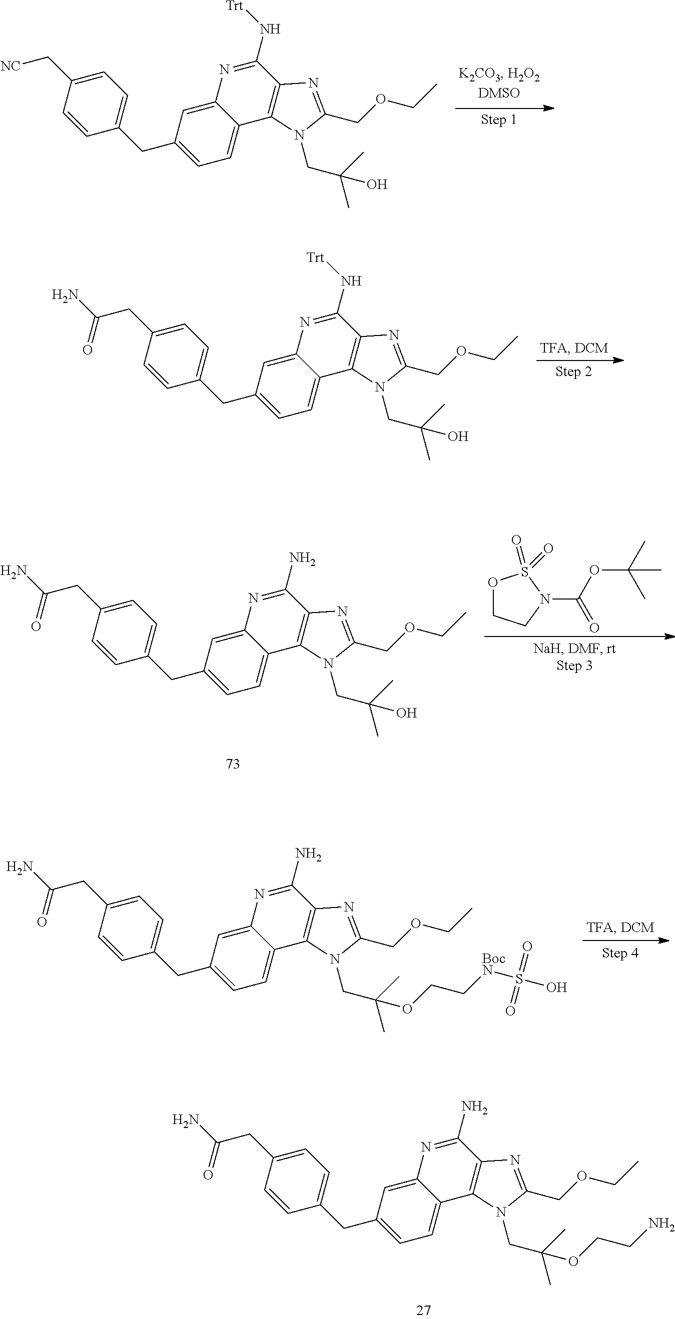

### Step 1: 2-(4-((2-(ethoxymethyl)-1-(2-hydroxy-2-methylpropyl)-4-(tritylamino)-1H-imidazo[4,5-c]quinolin-7-yl)methyl)phenyl)acetamide

To a solution of 2-(4-((2-(ethoxymethyl)-1-(2-hydroxy-2-methylpropyl)-4-(tritylamino)-1H-imidazo[4,5-c]quinolin-7-yl)methyl)phenyl)acetonitrile (200 mg, 0.29 mmol) in DMSO was added H₂O₂(2 mL) and K₂CO₃ (140 mg) at 0° C. The reaction mixture was warmed to RT and stirred for 30 minutes. LCMS showed the reaction was completed. The solution was diluted with water and extracted with EA (30 mL*3). The combined organic layers were dried over Na₂SO₄, concentrated to give the crude title compound. The crude product was purified by silica gel column chromatography (PE:EA 1:1) to give the title compound (160 mg, 77.9% yield) as a yellow solid.

LCMS (ESI) calcd for C₄₅H₄₅N₅O₃ [M+H]⁺ m/z=704.3, found 704.

### Step 2: 2-(4-((4-amino-2-(ethoxymethyl)-1-(2-hydroxy-2-methylpropyl)-1H-imidazo[4,5-c]quinolin-7-yl)methyl)phenyl)acetamide (Compound 73)

To a solution of 2-(4-((2-(ethoxymethyl)-1-(2-hydroxy-2-methylpropyl)-4-(tritylamino)-1H-imidazo[4,5-c]quinolin-7-yl)methyl)phenyl)acetamide (160 mg, 0.23 mmol) in DCM (3 mL) was added TFA (1 mL) dropwise at room temperature. The reaction mixture was stirred at room temperature for 3 hrs. The solution was removed under reduced pressure to afford the crude product. The residue was purified by Prep-HPLC (column: Gemini-C18 150×21.2 mm, 5 um; mobile phase: ACN-H2O (0.1% FA); gradient: 10-35) to afford Compound 73 (3.45 mg, 13.3% yield) as white solid.

LCMS (ESI) calcd for C₂₆H₃₁N₅O₃ [M+H]⁺ m/z=462.2, found 462.

¹H NMR (400 MHz, DMSO-d₆) δ 8.18 (d, J=8.6 Hz, 1H), 8.14 (s, 1H), 7.41 (d, J=14.7 Hz, 2H), 7.23-7.16 (m, 4H), 7.08 (d, J=7.4 Hz, 1H), 6.83 (s, 2H), 4.87 (s, 2H), 4.63 (s, 2H), 4.01 (s, 2H), 3.50 (q, J=7.0 Hz, 2H), 1.12 (m, 9H).

### Step 3: (2-((1-(4-amino-7-(4-(2-amino-2-oxoethyl)benzyl)-2-(ethoxymethyl)-1H-imidazo[4,5-c]quinolin-1-yl)-2-methylpropan-2-yl)oxy)ethyl)(tert-butoxycarbonyl)sulfamic acid

A solution of NaH (33 mg, 1.3 mmol, 60%) in DMF (2 mL) was stirred at 0° C. for 5 minutes. Then 2-(4-((4-amino-2-(ethoxymethyl)-1-(2-hydroxy-2-methylpropyl)-1H-imidazo[4,5-c]quinolin-7-yl)methyl)phenyl)acetamide (120 mg, 0.2603 mmol) was added to the mixture. The reaction mixture was stirred for 30 minutes at RT. tert-butyl 1,2,3-oxathiazolidine-3-carboxylate 2,2-dioxide (87 mg, 0.3904 mmol) was added to the mixture at 0° C. The mixture was stirred at rt for 16 hrs. The resulting mixture was diluted with water (30 mL) and extracted with EtOAc (30 mL×3). The combine organic phases were washed with brine, dried over Na₂SO₄, concentrated and purified by silica gel column chromatography (PE:EA 1:1) to give the product (50 mg, 28.0% yield) as a white solid.

LCMS (ESI) calcd for C₃₃H₄₄N₆O₈S [M+H]⁺ m/z=685.2, found 685.

### Step 4: 2-[4-({4-amino-1-[2-(2-aminoethoxy)-2-methylpropyl]-2-(ethoxymethyl)imidazo[4,5-c]quinolin-7-yl}methyl)phenyl]acetamide (Compound 27)

To a solution of (2-((1-(4-amino-7-(4-(2-amino-2-oxoethyl)benzyl)-2-(ethoxymethyl)-1H-imidazo[4,5-c]quinolin-1-yl)-2-methylpropan-2-yl)oxy)ethyl)(tert-butoxycarbonyl)sulfamic acid (50 mg, 0.07 mmol) in DCM (3 mL) was added a solution of TFA (1 mL) dropwise. The reaction mixture was stirred at RT for 3 hrs. The solution was removed under reduced pressure to afford the crude product. The residue was purified by Prep-HPLC (column: Gemini-C18 150×21.2 mm, 5 um; mobile phase: ACN-H₂O (0.05% NH₃); gradient: 30-90) to afford Compound 27 (14.22 mg, 38% yield) as white solid.

LCMS (ESI) calcd for C₁₅H₂₄N₈O₂ [M+H]⁺ m/z=505.2, found 505.

¹H NMR (400 MHz, DMSO-d₆) δ 8.21 (d, J=8.6 Hz, 1H), 8.14 (s, 1H), 7.44 (s, 1H), 7.39 (s, 1H), 7.19 (s, 4H), 7.08 (d, J=8.4 Hz, 1H), 6.84 (s, 1H), 6.65 (s, 1H), 4.75 (s, 2H), 4.02 (s, 2H), 3.51 (q, J=7.0 Hz, 2H), 3.39-3.35 (m, 4H), 3.33 (s, 2H), 2.77 (t, J=5.4 Hz, 2H), 1.14 (m, 9H).

### Example 17

### Synthesis of Compound 32

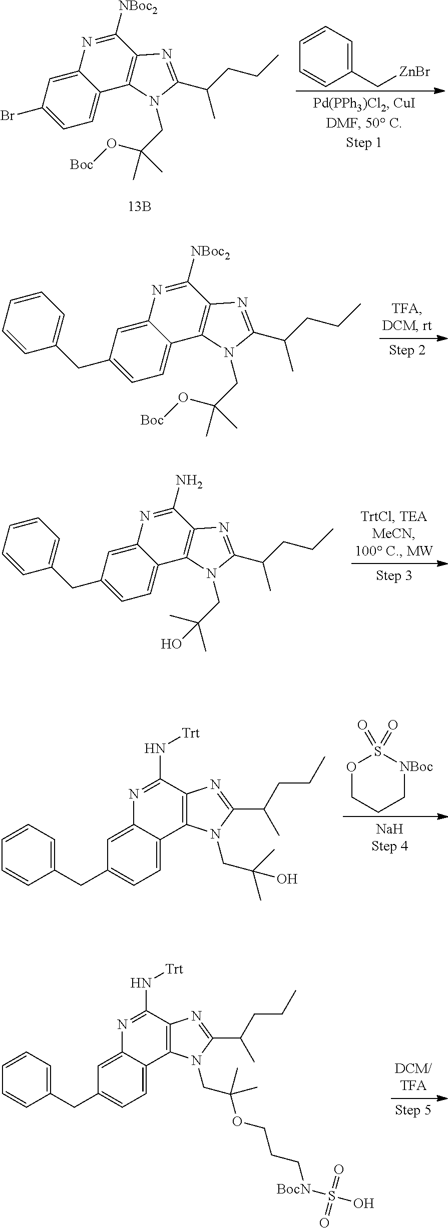

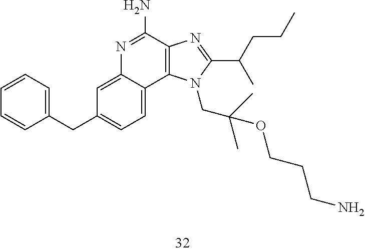 [SMILES: CCCC(C)c1nc2c(N)nc3cc(Cc4ccccc4)ccc3c2n1CC(C)(C)OCCCN]

### Step 1: Preparation of di-tert-butyl (7-benzyl-1-(2-((tert-butoxycarbonyl)oxy)-2-methylpropyl)-2-(pentan-2-yl)-1H-imidazo[4,5-c]quinolin-4-yl)iminodicarbonate

To a solution of di-tert-butyl (7-bromo-1-(2-((tert-butoxycarbonyl)oxy)-2-methylpropyl)-2-(pentan-2-yl)-1H-imidazo[4,5-c]quinolin-4-yl)iminodicarbonate (500 mg, 0.71 mmol), Pd(PPh₃)Cl₂(50 mg, 0.07 mmol) and CuI (27 mg, 0.14 mmol) in DMF (3 mL) was added a solution of benzylzinc(II) bromide in DMF (10 mL). The mixture was stirred at 50° C. for 0.5 h. LCMS showed the reaction was completed. The resulting mixture was diluted with water (60 mL) and extracted with EA (40 mL×3). The organic phases were washed with brine, dried over Na₂SO₄, concentrated to give the crude title compound. The residue was purified by combi-flash (PE:EA1:1) to afford di-tert-butyl (7-benzyl-1-(2-((tert-butoxycarbonyl)oxy)-2-methylpropyl)-2-(pentan-2-yl)-1H-imidazo[4,5-c]quinolin-4-yl)iminodicarbonate (450 mg, 88.58% yield) as yellow solid.

LCMS (ESI) calcd for C₄₁H₅₆N₄O₇ [M+H]⁺ m/z=717.4, found 717.

### Step 2: Preparation of 1-(4-amino-7-benzyl-2-(pentan-2-yl)-1H-imidazo[4,5-c]quinolin-1-yl)-2-methylpropan-2-ol

To a solution of di-tert-butyl (7-benzyl-1-(2-((tert-butoxycarbonyl)oxy)-2-methylpropyl)-2-(pentan-2-yl)-1H-imidazo[4,5-c]quinolin-4-yl)iminodicarbonate (450 mg, 0.63 mmol) in DCM (6 mL) was added TFA (2 mL) dropwise at 0° C. The mixture was stirred at rt for 2 hours. The resulting mixture was concentrated in vacuo. The residue was adjusted to pH=8 with saturated sodium bicarbonate in aqueous solution. The resulting mixture was extracted with EA (30 mL×3). The combine organic phases were washed with brine, dried over Na₂SO₄, concentrated to give the crude title compound.

LCMS (ESI) calcd for C₂₆H₃₂N₄₀ [M+H]⁺ m/z=417.3, found 417.

### Step 3: Preparation of 1-(7-benzyl-2-(pentan-2-yl)-4-(tritylamino)-1H-imidazo[4,5-c]quinolin-1-yl)-2-methylpropan-2-ol

A mixture of 1-(4-amino-7-benzyl-2-(pentan-2-yl)-1H-imidazo[4,5-c]quinolin-1-yl)-2-methylpropan-2-ol (200 mg, 0.48 mmol), TrtCl (200 mg, 0.72 mmol) and Et₃N (145 mg, 1.44 mmol) in MeCN (4 mL) was heated at 100° C. in a microwave reactor for 0.5 h under an atmosphere of N₂. LCMS showed the reaction was completed. The resulting mixture was cooled to 0° C. The precipitate was collected by filtration to afford 1-(7-benzyl-2-(pentan-2-yl)-4-(tritylamino)-1H-imidazo[4,5-c]quinolin-1-yl)-2-methylpropan-2-ol (130 mg, 47.79% yield) as white solid.

LCMS (ESI) calcd for C₄₅H₄₆N₄₀ [M+H]⁺ m/z=659.4, found 659.

### Step 4: Preparation of (3-((1-(7-benzyl-2-(pentan-2-yl)-4-(tritylamino)-1H-imidazo[4,5-c]quinolin-1-yl)-2-methylpropan-2-yl)oxy)propyl)(tert-butoxycarbonyl)sulfamic acid

To a solution of NaH (40 mg, 60% ) in DMF (1 mL) was added a solution of 1-(7-benzyl-2-(pentan-2-yl)-4-(tritylamino)-1H-imidazo[4,5-c]quinolin-1-yl)-2-methylpropan-2-ol (130 mg, 0.20 mmol) in DMF (2 mL) dropwise. The mixture was stirred at RT for 1 h. Then tert-butyl 1,2,3-oxathiazinane-3-carboxylate 2,2-dioxide (233 mg, 0.99 mmol) was added to the mixture. The mixture was stirred at RT for 2 hours. LCMS showed the reaction was completed. The resulting mixture was quenched with water (20 mL) and extracted with EA (30 mL×3). The combined organic phases were washed with brine, dried over Na₂SO₄, concentrated to give the crude title compound. The residue was purified by combi-flash (PE:EA1:2) to afford (3-((1-(7-benzyl-2-(pentan-2-yl)-4-(tritylamino)-1H-imidazo[4,5-c]quinolin-1-yl)-2-methylpropan-2-yl)oxy)propyl)(tert-butoxycarbonyl) sulfamic acid (70 mg, 39.59% yield) as yellow solid.

LCMS (ESI) calcd for C₅₃H₆₁N₅O₆S [M+H]⁺ m/z=896.4, found 896.

### Step 5: Preparation of 1-(2-(3-aminopropoxy)-2-methylpropyl)-7-benzyl-2-(pentan-2-yl)-1H-imidazo[4,5-c]quinolin-4-amine (Compound 32)

To a solution of (3-((1-(7-benzyl-2-(pentan-2-yl)-4-(tritylamino)-1H-imidazo[4,5-c]quinolin-1-yl)-2-methylpropan-2-yl)oxy)propyl)(tert-butoxycarbonyl)sulfamic acid (70 mg, 0.078 mmol) in DCM (3 mL) was added TFA (1 mL) dropwise at 0° C. The mixture was stirred at RT for 2 hours. LCMS showed the reaction was completed. The reaction was concentrated and purified by Prep-HPLC (Column: Gemini-C18, 150×21.2 mm, 5 um; Mobile Phase: ACN-H₂O (0.05% FA), Gradient: 10-40) to give Compound 32 (6.23 mg, 16.84% yield) as white solid.

LCMS (ESI) calcd for C₂₉H₃₉N₅O [M+H]⁺ m/z=474.3, found 474.

¹H NMR (400 MHz, DMSO-d₆) δ 8.41 (s, 1H), 8.16 (d, J=8.5 Hz, 1H), 7.40 (s, 1H), 7.36-7.24 (m, 4H), 7.24-7.15 (m, 1H), 7.07 (d, J=8.5 Hz, 1H), 6.36 (s, 2H), 4.61 (d, J=136.5 Hz, 4H), 4.04 (s, 2H), 3.28 (dd, J=11.3, 6.1 Hz, 4H), 1.54-1.43 (m, 2H), 1.43-0.97 (m, 12H), 0.90-0.76 (m, 3H).

Compound 33 was prepared by similar method as described in Example 16 using corresponding reagents.

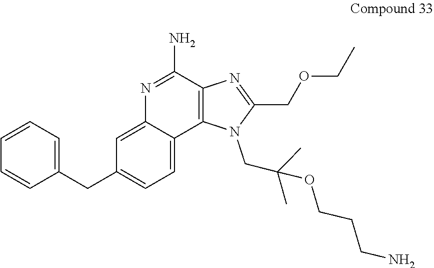 [SMILES: CCOCc1nc2c(N)nc3cc(Cc4ccccc4)ccc3c2n1CC(C)(C)OCCCN]

LCMS (ESI) calcd for C₂₇H₃₅N₅O₂ [M+H]⁺ m/z=462.3, found 462.

¹H NMR (400 MHz, DMSO-d₆) δ 8.22 (d, J=8.5 Hz, 1H), 7.43 (s, 1H), 7.35-7.26 (m, 4H), 7.25-7.17 (m, 1H), 7.14 (dd, J=8.5, 1.5 Hz, 1H), 7.06 (s, 2H), 4.88 (s, 2H), 4.64 (s, 2H), 4.06 (s, 2H), 3.50 (dd, J=14.0, 7.0 Hz, 6H), 1.31-0.92 (m, 9H).

### Example 18

### Synthesis of Compounds 34 and 35

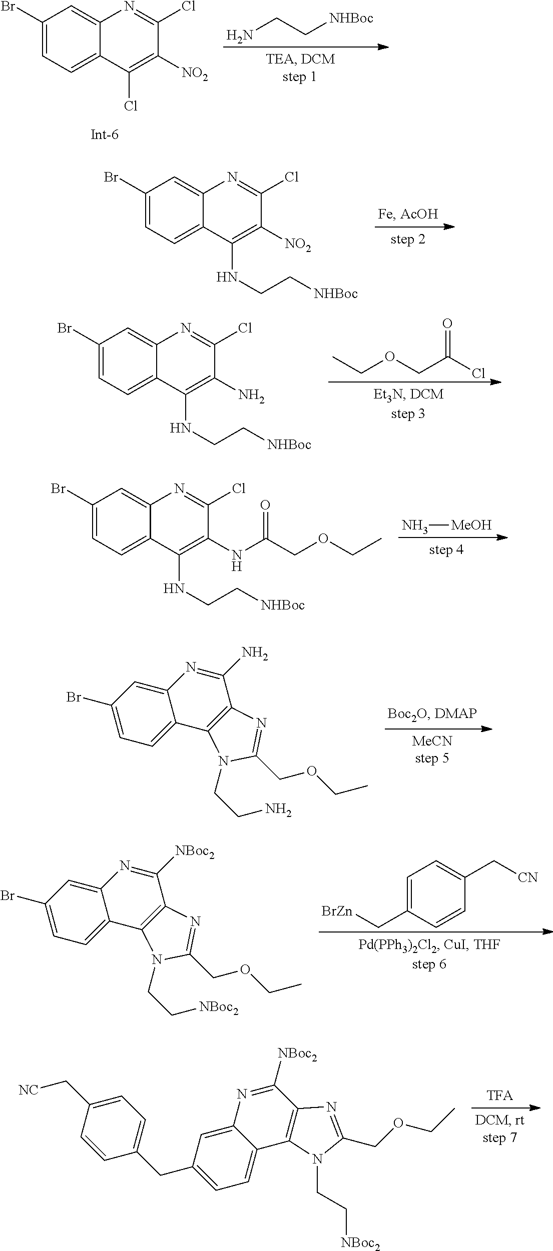

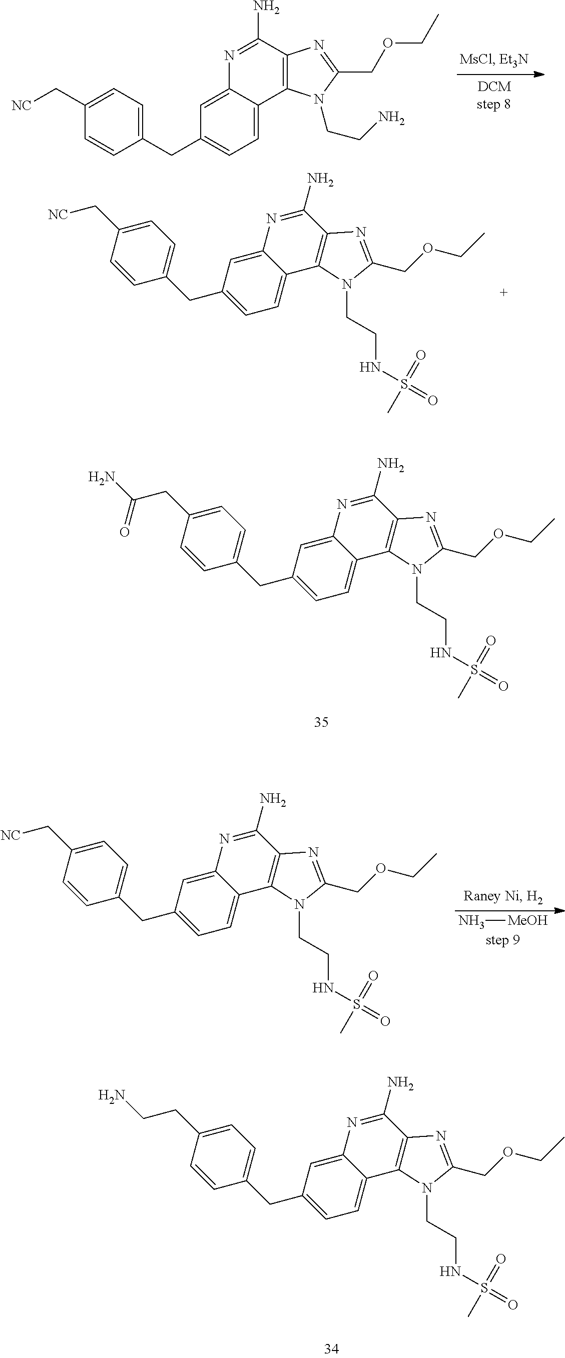

### Step 1: Preparation of tert-butyl N-{2-[(7-bromo-2-chloro-3-nitroquinolin-4-yl)amino]ethyl}carbamate

To a solution of 7-bromo-2,4-dichloro-3-nitroquinoline (3 g, 9.32 mmol) and Et₃N (1.41 g, 13.95 mmol) in DCM (30 mL) was added tert-butyl N-(2-aminoethyl)carbamate (1.79 g, 11.16 mmol) dropwise at 0° C. The mixture was stirred room temperature for 4 hours. LCMS showed the reaction was completed. The resulting mixture was diluted with water (20 mL) and extracted with DCM (20 mL×3). The organic phases were dried over Na₂SO₄, concentrated to give the crude tert-butyl (2-((7-bromo-2-chloro-3-nitroquinolin-4-yl)amino)ethyl)carbamate (2.5 g) which was used in next reaction without further purification.

LCMS (ESI) calcd for C₁₆H₁₅BrClN₄O₄[M+H]⁺ m/z=445.0, found 445.0.

### Step 2: Preparation of tert-butyl (2-((3-amino-7-bromo-2-chloroquinolin-4-yl)amino)ethyl)carbamate

To a solution of tert-butyl (2-((7-bromo-2-chloro-3-nitroquinolin-4-yl)amino)ethyl)carbamate (2.5 g, 5.6 mmol) in AcOH (40 mL) was added Fe (3.13 g, 56 mmol). The reaction mixture was stirred at room temperature for 6 hours. LCMS showed the reaction was completed. The resulting mixture was filtered through celite, and the filtrate was concentrated in vacuo. The residue was diluted with water (50 mL) and extracted with EtOAc (50 mL×3). The organic phases were washed with brine, dried over Na₂SO₄, concentrated and purified by column chromatography on silica gel (PE/EtOAc=1/2) to give tert-butyl (2-((3-amino-7-bromo-2-chloroquinolin-4-yl)amino)ethyl)carbamate (2 g, 85.7% yield) as yellow solid.

LCMS (ESI) calcd for C₁₆H₂₀BrClN₄O₂[M+H]⁺ m/z=415.0, found 415.0.

### Step 3: Preparation of tert-butyl (2-((7-bromo-2-chloro-3-(2-ethoxyacetamido)quinolin-4-yl)amino)ethyl)carbamate

To a stirred solution of tert-butyl (2-((3-amino-7-bromo-2-chloroquinolin-4-yl)amino)ethyl)carbamate (2 g, 4.8 mmol) in DCM (30 mL) was added 2-ethoxyacetyl chloride (0.88 g, 7.2 mmol) dropwise at 0° C. The reaction mixture was stirred at room temperature for 3 hours. The organic phases were washed with brine, dried over Na₂SO₄, concentrated, and purified by column chromatography on silica gel (PE/EtOAc=1/3) to give tert-butyl (2-((7-bromo-2-chloro-3-(2-ethoxyacetamido)quinolin-4-yl)amino)ethyl)carbamate (1.5 g, 62.5% yield) as a yellow solid.

LCMS (ESI) calcd for C₂₀H₂₆BrClN₄O₄[M+H]⁺ m/z=501.1, found 501.0.

### Step 4: Preparation of 1-(2-aminoethyl)-7-bromo-2-(ethoxymethyl)-1H-imidazo[4,5-c]quinolin-4-amine

A solution of tert-butyl (2-((7-bromo-2-chloro-3-(2-ethoxyacetamido)quinolin-4-yl)amino)ethyl)carbamate (1.5 g, 3.2 mmol) in NH₃-MeOH (7M, 20 mL) was heated at 160° C. for 8 hours in a high-pressure reactor. After cooling to ambient temperature, the reaction mixture was concentrated to give the crude 1-(2-aminoethyl)-7-bromo-2-(ethoxymethyl)-1H-imidazo[4,5-c]quinolin-4-amine (600 mg) as yellow solid which was used in the next reaction without further purification.

LCMS (ESI) calcd for C₁₅H₁₈BrN₅O [M+H]⁺ m/z=364.1, found 364.0.

### Step 5: Preparation of di-tert-butyl (1-(2-(bis(tert-butoxycarbonyl)amino)ethyl)-7-bromo-2-(ethoxymethyl)-1H-imidazo[4,5-c]quinolin-4-yl)iminodicarbonate

To a solution of 1-(2-aminoethyl)-7-bromo-2-(ethoxymethyl)-1H-imidazo[4,5-c]quinolin-4-amine (600 mg, 1.65 mmol) and DMAP (80.5 mg, 0.65 mmol) in MeCN (20 mL) was added Boc₂O (1.8 g, 8.24 mmol) at room temperature. The mixture was heated at 75° C. for 4 hours. The resulting mixture was diluted with water (50 mL) and extracted with EA (50 mL×3). The combined organic phases were washed with brine, dried over Na₂SO₄, concentrated and purified by column chromatography on silica gel (PE/EtOAc=2/1) to give di-tert-butyl (1-(2-(bis(tert-butoxycarbonyl)amino)ethyl)-7-bromo-2-(ethoxymethyl)-1H-imidazo[4,5-c]quinolin-4-yl)iminodicarbonate (550 mg, 42.7% yield) as a yellow solid.

LCMS (ESI) calcd for C₃₅H₅₀BrN₅O₉[M+H]⁺ m/z=764.3, found 764.0.

### Step 6: Preparation of di-tert-butyl (1-(2-(bis(tert-butoxycarbonyl)amino)ethyl)-7-(4-(cyanomethyl)benzyl)-2-(ethoxymethyl)-1H-imidazo[4,5-c]quinolin-4-yl)iminodicarbonate

To a solution of di-tert-butyl (1-(2-(bis(tert-butoxycarbonyl)amino)ethyl)-7-bromo-2-(ethoxymethyl)-1H-imidazo[4,5-c]quinolin-4-yl)iminodicarbonate (550 mg, 0.72 mmol), Pd(PPh₃)Cl₂ (50.48 mg, 0.072 mmol) and CuI (54.79 mg, 0.14 mmol) in DMF (3 mL) was added benzylzinc(II) bromide (594.38 mg, 2.16 mmol) in DMF (3 mL). The mixture was heated at 50′C for 2 hours. LCMS showed the reaction was completed. The resulting mixture was diluted with water (20 mL) and extracted with EtOAc (20 mL×3). The organic phases were washed with brine, dried over Na₂SO₄, concentrated, and purified by column chromatography on silica gel (PE/EtOAc=1/2) to give di-tert-butyl (1-(2-(bis(tert-butoxycarbonyl)amino)ethyl)-7-(4-(cyanomethyl)benzyl)-2-(ethoxymethyl)-1H-imidazo[4,5-c]quinolin-4-yl)iminodicarbonate (300 mg, 52% yield) as a yellow solid.

LCMS (ESI) calcd for C₄₄H₅₈N₆O₉ [M+H]⁺ m/z=814.4, found 814.0.

### Step 7: Preparation of 2-(4-((4-amino-1-(2-aminoethyl)-2-(ethoxymethyl)-1H-imidazo[4,5-c]quinolin-7-yl)methyl)phenyl)acetonitrile

To a solution of di-tert-butyl (1-(2-(bis(tert-butoxycarbonyl)amino)ethyl)-7-(4-(cyanomethyl)benzyl)-2-(ethoxymethyl)-1H-imidazo[4,5-c]quinolin-4-yl)iminodicarbonate (300 mg, 0.37 mmol) in DCM (4 mL) was added TFA (2 mL) dropwise at 0° C. The mixture was stirred at room temperature for 2 hours. The resulting mixture was concentrated in vacuo. The residue was diluted was EtOAc (20 mL) and adjusted to pH 7-8 with saturated aqueous sodium bicarbonate. The organic phase was washed with brine, dried over Na₂SO₄ and concentrated to give the crude 2-(4-((4-amino-1-(2-aminoethyl)-2-(ethoxymethyl)-1H-imidazo[4,5-c]quinolin-7-yl)methyl)phenyl)acetonitrile (140 mg) as colorless oil which was used in the next reaction without further purification.

LCMS (ESI) calcd for C₂₄H₂₆N₆O [M+H]⁺ m/z=415.2, found 415.0.

### Step 8: Preparation of N-(2-(4-amino-7-(4-(cyanomethyl)benzyl)-2-(ethoxymethyl)-1H-imidazo[4,5-c]quinolin-1-yl)ethyl)methanesulfonamide (9) and 2-(4-((4-amino-2-(ethoxymethyl)-1-(2-(methylsulfonamido)ethyl)-1H-imidazo[4,5-c]quinolin-7-yl)methyl)phenyl)acetamide (Compound 35)

To a solution of 2-(4-((4-amino-1-(2-aminoethyl)-2-(ethoxymethyl)-1H-imidazo[4,5-c]quinolin-7-yl)methyl)phenyl)acetonitrile (140 mg, 0.34 mmol), Et₃N (51.28 mg, 0.51 mmol) in DCM (3 mL) was added methanesulfonic anhydride (64.72 mg, 0.37 mmol) in DCM (2 mL) dropwise at 0° C. The reaction mixture was stirred at room temperature for 30 minutes. LCMS showed the reaction was completed. The resulting mixture was diluted with water (10 mL) and extracted with DCM (10 mL×3). The organic phases were washed with brine, dried over Na₂SO₄, concentrated and purified by prep-HPLC (Column: Gemini-C18, 150×21.2 mm, 5 um; Mobile Phase: ACN-H₂O (0.1% FA), Gradient: 20%-35%) to give N-(2-(4-amino-7-(4-(cyanomethyl)benzyl)-2-(ethoxymethyl)-1H-imidazo[4,5-c]quinolin-1-yl)ethyl)methanesulfonamide (100 mg, 59.7% yield) as white solid and 2-(4-((4-amino-2-(ethoxymethyl)-1-(2-(methylsulfonamido)ethyl)-1H-imidazo[4,5-c]quinolin-7-yl)methyl)phenyl)acetamide (Compound 35, 10 mg, 5.76% yield) as a white solid.

LCMS (ESI) calcd for C₂₅H₃₀N₆O₄S [M+H]⁺ m/z=511.2, found 511.0.

¹H NMR (400 MHz, DMSO-d₆) δ 9.76 (s, 1H), 8.39 (s, 3H), 8.11 (d, J=8.4 Hz, 1H), 7.42 (s, 1H), 7.30 (q, J=8.4 Hz, 4H), 7.10 (d, J=8.5 Hz, 1H), 6.55 (s, 2H), 4.71 (s, 4H), 4.06 (s, 2H), 3.99 (s, 2H), 3.69 (s, 3H), 3.63-3.44 (m, 1H), 1.16 (t, J=7.0 Hz, 3H).

### Step 9: Preparation of N-(2-(4-amino-7-(4-(2-aminoethyl)benzyl)-2-(ethoxymethyl)-1H-imidazo[4,5-c]quinolin-1-yl)ethyl)methanesulfonamide (Compound 34)

To a solution of N-(2-(4-amino-7-(4-(cyanomethyl)benzyl)-2-(ethoxymethyl)-1H-imidazo[4,5-c]quinolin-1-yl)ethyl)methanesulfonamide (100 mg, 0.2 mmol) which was obtained form above step in NH₃-MeOH (7M, 3 mL) was added a suspension of Raney Ni in NH₃-MeOH (7M, 2 mL). The reaction mixture was stirred under an atmosphere of hydrogen at room temperature for 1 hour. LCMS showed the reaction was completed. The reaction mixture was filtered through celite. The filtrate was concentrated under vacuum and purified by prep-HPLC (Column: Gemini-C18, 150×21.2 mm, 5 um; Mobile Phase: ACN-H₂O (0.1% FA), Gradient: 10%-45%) to give N-(2-(4-amino-7-(4-(2-aminoethyl)benzyl)-2-(ethoxymethyl)-1H-imidazo[4,5-c]quinolin-1-yl)ethyl)methanesulfonamide (Compound 34, 20 mg, 20.1% yield) as a white solid.

LCMS (ESI) calcd for C₂₅H₃₂N₆O₃S [M+H]⁺ m/z=497.2, found 497.0.

¹H NMR (400 MHz, CD₃OD, ppm) δ 8.41 (s, 1H), 8.09 (d, J=8.5 Hz, 1H), 7.45 (s, 1H), 7.25 (dd, J=8.5, 1.5 Hz, 1H), 7.16 (dd, J=22.6, 8.1 Hz, 4H), 4.80 (s, 2H), 4.71 (t, J=6.7 Hz, 2H), 4.03 (s, 2H), 3.58 (q, J=7.0 Hz, 2H), 3.51 (t, J=6.7 Hz, 2H), 3.11-2.97 (m, 2H), 2.91-2.79 (m, 2H), 2.78 (s, 3H), 1.17 (t, J=7.0 Hz, 3H).

### Example 19

### Synthesis of Compounds 36-38

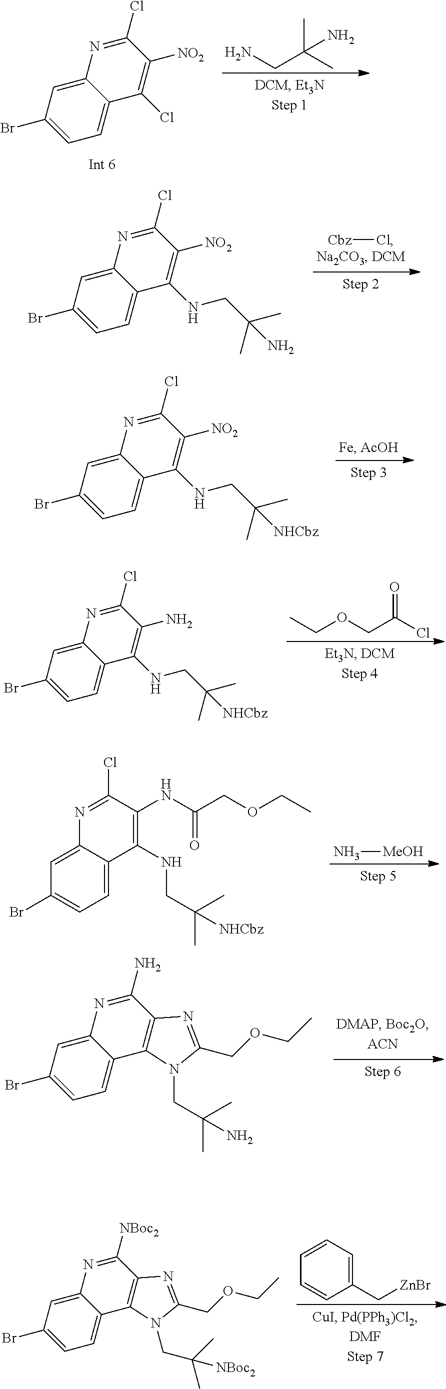

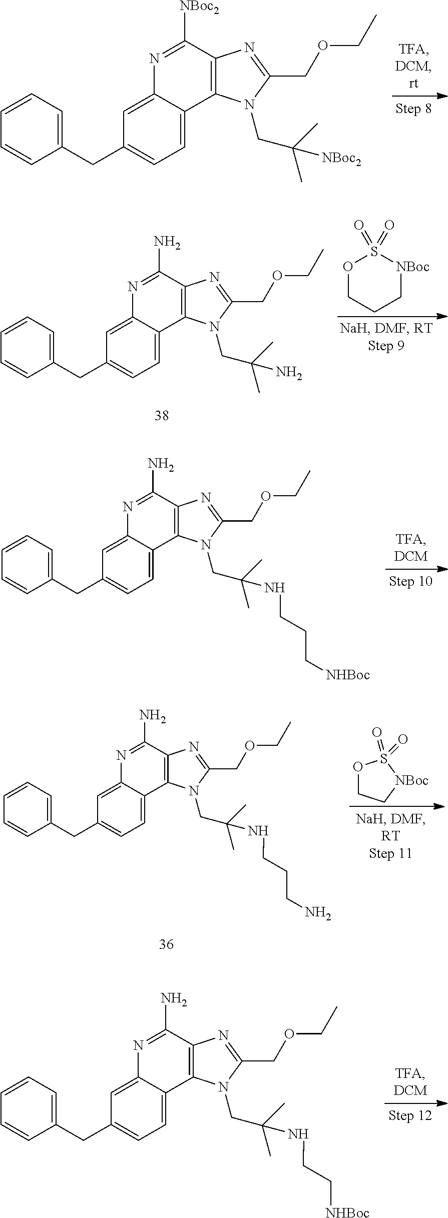

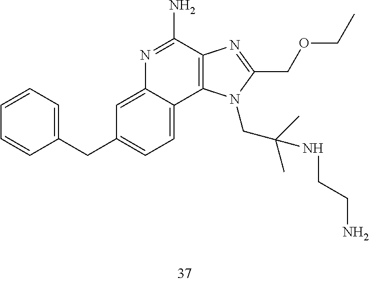 [SMILES: CCOCc1nc2c(N)nc3cc(Cc4ccccc4)ccc3c2n1CC(C)(C)NCCN]

### Step 1: N-(2-amino-2-methylpropyl)-7-bromo-2-chloro-3-nitroquinolin-4-amine

To a solution of 7-bromo-2,4-dichloro-3-nitroquinoline (6 g, 18.6 mmol) and TEA (2.82 g, 27.8 mmol] in DCM (60 mL) was added a solution of 2-methylpropane-1,2-diamine (1.64 g, 18.6 mmol) dropwise. The reaction mixture was stirred at RT for 16 hrs. LCMS showed the reaction was completed. The resulting mixture was diluted with water (100 mL) and extracted with EtOAc (100 mL×3). The combine organic phases were washed with brine, dried over Na₂SO₄, concentrated to give the crude. The residue was purified by silica gel column chromatography (PE:EA 1:1) to give the title compound (5 g, 64.5% yield) as a yellow solid.

LCMS (ESI) calcd for C₁₃H₁₄BrClN₄O₂[M+H]⁺ m/z=373, found 373.

### Step 2: benzyl (1-((7-bromo-2-chloro-3-nitroquinolin-4-yl)amino)-2-methylpropan-2-yl)carbamate

To a solution of N-(2-amino-2-methylpropyl)-7-bromo-2-chloro-3-nitroquinolin-4-amine (4 g, 10.7 mmol) and Na₂CO₃ (20 mL, 2 M) in DCM (30 mL) was added a solution of CbzCl (2 g, 11.7 mmol) in DCM dropwise under 0° C. The reaction mixture was stirred at RT for overnight. LCMS showed the reaction was completed. The resulting mixture was diluted with water (100 mL) and extracted with DCM (80 mL×3). The combine organic phases were washed with brine, dried over Na₂SO₄, concentrated to give the crude. The residue was purified by silica gel column chromatography (PE:EA 5:1) to give the title compound (4 g, 66.3% yield) as a white solid.

LCMS (ESI) calcd for C₂₁H₂₀BrClN₄O₄[M+H]⁺ m/z=507.1, found 507.

### Step 3: benzyl (1-((3-amino-7-bromo-2-chloroquinolin-4-yl)amino)-2-methylpropan-2-yl)carbamate

To a solution of benzyl (1-((7-bromo-2-chloro-3-nitroquinolin-4-yl)amino)-2-methylpropan-2-yl)carbamate (4 g, 7.9 mmol) in AcOH (50 mL) was added Fe (4.5 g, 79 mmol) at RT. The reaction mixture was stirred at RT for 5 hrs. The mixture was filtered through celite and the filtrate was concentrated under vacuum. The resulting mixture was diluted with water (100 mL) and extracted with DCM (80 mL×3). The combine organic phases were washed with brine, dried over Na₂SO₄, concentrated to give the crude. The residue was purified by silica gel column chromatography (PE:EA 3:1) to give the title compound (3.5 g, 83.5% yield) as a yellow solid.

LCMS (ESI) calcd for C₂₁H₂₂BrClN₄O₂ [M+H]⁺ m/z=477.1, found 477.

### Step 4: benzyl (1-((7-bromo-2-chloro-3-(2-ethoxyacetamido)quinolin-4-yl)amino)-2-methylpropan-2-yl)carbamate

To a solution of benzyl (1-((3-amino-7-bromo-2-chloroquinolin-4-yl)amino)-2-methylpropan-2-yl)carbamate (3.5 g, 7.3 mmol) and TEA (0.98 g, 8.0 mmol) in DCM (50 mL) was added a solution of 2-ethoxyacetyl chloride (2.22 g, 21.9 mmol) at RT. The reaction mixture was stirred at RT for 16 hrs. LCMS showed the reaction was completed. The resulting mixture was diluted with water (100 mL) and extracted with DCM (80 mL×3). The combine organic phases were washed with brine, dried over Na₂SO₄, concentrated to give the crude. The residue was purified by silica gel column chromatography (PE:EA 3:1) to give the title compound (3.5 g, 76.7% yield) as a white solid.

LCMS (ESI) calcd for C₂₅H₂₈BrClN₄O₄[M+H]⁺ m/z=563.1, found 563.

### Step 5: 1-(2-amino-2-methylpropyl)-7-bromo-2-(ethoxymethyl)-1H-imidazo[4,5-c]quinolin-4-amine

A solution of benzyl (1-((7-bromo-2-chloro-3-(2-ethoxyacetamido)quinolin-4-yl)amino)-2-methylpropan-2-yl)carbamate (3.5 g, 6.2 mmol) in NH₃-MeOH (20 mL) was stirred at 160° C. for 8 hours in a high-pressure reactor. After cooling to ambient temperature, the mixture was concentrated in vacuo to give the crude without purification.

LCMS (ESI) calcd for C₁₇H₂₂BrN₅O [M+H]⁺ m/z=392.1, found 392.

### Step 6: di-tert-butyl (1-(2-(bis(tert-butoxycarbonyl)amino)-2-methylpropyl)-7-bromo-2-(ethoxymethyl)-1H-imidazo[4,5-c]quinolin-4-yl)iminodicarbonate

To a solution of 1-(2-amino-2-methylpropyl)-7-bromo-2-(ethoxymethyl)-1H-imidazo[4,5-c]quinolin-4-amine (2.5 g, 6.3 mmol) in ACN (25 mL) was added DMAP (311 mg, 2.52 mmol) and Boc₂O (14 g, 63 mmol). The reaction mixture was stirred at 75° C. for 4 hours. It was concentrated and diluted with EtOAc. The organic phase was washed with brine, dried over Na₂SO₄, filtered, and concentrated in vacuo. The residue was purified by silica gel column chromatography (PE:EA 3:1) to afford the title compound (3 g, 60.1% yield) as a yellow solid.

LCMS (ESI) calcd for C₃₇H₅₄BrN₅O₉[M+H]⁺ m/z=792.3, found 792.

### Step 7: di-tert-butyl (7-benzyl-1-(2-(bis(tert-butoxycarbonyl)amino)-2-methylpropyl)-2-(ethoxymethyl)-1H-imidazo[4,5-c]quinolin-4-yl)iminodicarbonate

A mixture of Zn powder (6.0 g, 93 mmol) and I₂ (750 mg, 3 mmol) in a 250 mL three-necked flask was evacuated and backfilled with N₂ three times and then charged with N₂. The flask was heated carefully with a hot air blower until I₂ was sublimated. Then a solution of (bromomethyl)benzene (5 g, 29.6 mmol) in DMF (30 mL) was added intermediately via a syringe. The mixture was stirred for 10 minutes while keeping the temperature was about 70° C. Then the heater and stirrer were removed. The upper layer clear solution was added dropwise into a stirred solution of di-tert-butyl (1-(2-(bis(tert-butoxycarbonyl)amino)-2-methylpropyl)-7-bromo-2-(ethoxymethyl)-1H-imidazo[4,5-c]quinolin-4-yl)iminodicarbonate (3 g, 3.78 mmol), CuI (300 mg, 1.6 mmol) and Pd(pph₃)₂Cl₂ (400 mg, 0.57 mmol) in DMF (10 mL) under an atmosphere of N₂. The mixture was heated at 50 TC for 1 hour. Then the mixture was quenched with water (150 mL) and extracted with EtOAc (100 mL×3). The combined organic layers were washed with water and brine, dried over Na₂SO₄, filtered, concentrated to give the crude. The residue was purified by silica gel column chromatography (PE:EA 5:1) to afford desired product (2 g, 65.8% yield) as a yellow solid.

LCMS (ESI) calcd for C₄₄H₁N₅O₉[M+H]⁺ m/z=804.4, found 804.

### Step 8: 1-(2-amino-2-methylpropyl)-7-benzyl-2-(ethoxymethyl)-1H-imidazo[4,5-c]quinolin-4-amine (Compound 38)

To a solution of di-tert-butyl (7-benzyl-1-(2-(bis(tert-butoxycarbonyl)amino)-2-methylpropyl)-2-(ethoxymethyl)-11H-imidazo[4,5-c]quinolin-4-yl)iminodicarbonate (100 mg, 0.13 mmol) in DCM (6 mL) was added TFA (2 mL) dropwise at room temperature. The reaction mixture was stirred at RT for 3 hours. The solution was removed under reduced pressure to afford the crude product. The residue was purified by Prep-HPLC (column: Gemini-C18 150×21.2 mm, 5 um; mobile phase: ACN-H₂O (0.1% NH₃—H₂O); gradient: 10-75) to afford Compound 38 (4.4 mg, 8% yield) as white solid.

LCMS (ESI) calcd for C₂₄H₂₉N₅O [M+H]⁺ m/z=404.2, found 404.

¹H-NMR (400 MHz, DMSO-d₆) δ 8.29 (d, J=8.5 Hz, 1H), 7.50 (s, 1H), 7.40-7.27 (m, 4H), 7.25-7.16 (m, 2H), 4.86 (m, 4H), 4.10 (s, 2H), 3.56 (s, 2H), 1.48-0.91 (m, 9H).

### Step 9: tert-butyl N-[3-({1-[4-amino-7-benzyl-2-(ethoxymethyl)imidazo[4,5-c]quinolin-1-yl]-2-methylpropan-2-yl}amino)propyl]carbamate

A solution of NaH (90 mg, 3.72 mmol, 60%) in DMF (3 mL) was stirred at 0° C. for 5 minutes. Then 1-(2-amino-2-methylpropyl)-7-benzyl-2-(ethoxymethyl)imidazo[4,5-c]quinolin-4-amine (Compound 38, 300 mg, 0.74 mmol) was added to the mixture. The reaction mixture was stirred for 30 minutes at RT. Tert-butyl 1,2,3-oxathiazinane-3-carboxylate 2,2-dioxide (318 mg, 1.33 mmol) was added to the mixture at 0° C. The mixture was stirred at rt for 16 hrs. The resulting mixture was diluted with water (30 mL) and extracted with EtOAc (30 mL×3). The combined organic phases were washed with brine, dried over Na₂SO₄, concentrated and purified by silica gel column chromatography (PE:EA 1:1) to give the title compound (50 mg, 28.0% yield) as a white solid.

LCMS (ESI) calcd for C₃₂H₄₄N₆O₃ [M+H]⁺ m/z=561.3, found 561.

### Step 10: NJ-(1-(4-amino-7-benzyl-2-(ethoxymethyl)-1H-imidazo[4,5-c]quinolin-1-yl)-2-methylpropan-2-yl)propane-1,3-diamine (Compound 36)

To a solution of tert-butyl (3-((1-(4-amino-7-benzyl-2-(ethoxymethyl)-1H-imidazo[4,5-c]quinolin-1-yl)-2-methylpropan-2-yl)amino)propyl)carbamate (50 mg, 0.09 mmol) in DCM (3 mL) was added TFA (1 mL) at 0° C. The reaction mixture was stirred at RT for 3 hrs. The solution was removed under reduced pressure to afford the crude product. The residue was purified by Prep-HPLC (column: Gemini-C18 150×21.2 mm, 5 um; mobile phase: ACN-H₂O (0.1% FA); gradient: 10-40) to afford Compound 36 (2 mg, 4.6% yield) as white solid.

LCMS (ESI) calcd for C₂₇H₃₆N₆O [M+H]⁺ m/z=461.3, found 461.

### Step 11 and step 12: Preparation of N1-(1-(4-amino-7-benzyl-2-(ethoxymethyl)-1H-imidazo[4,5-c]quinolin-1-yl)-2-methylpropan-2-yl)ethane-1,2-diamine (Compound 37)

Compound 37 was synthesized from Compound 38 by steps 11 and 12 (procedures for steps 11 and 12 were similar with steps 9 and 10).

LCMS (ESI) calcd for C₂₆H₃₄N₆O [M+H]⁺ m/z=447.2, found 447.

### Example 20

### Synthesis of Compound 39

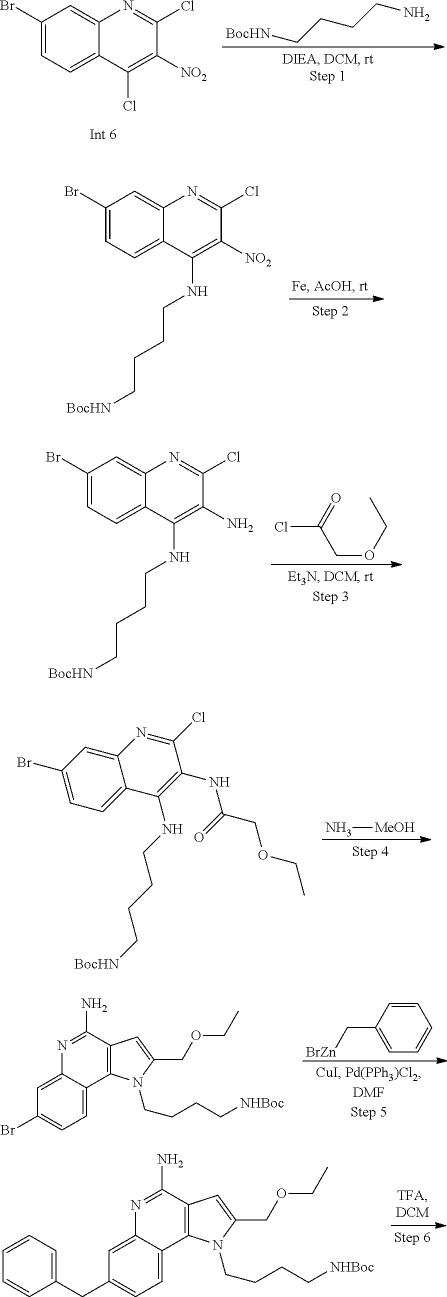

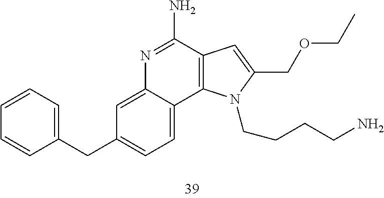 [SMILES: CCOCc1cc2c(N)nc3cc(Cc4ccccc4)ccc3c2n1CCCCN]

### Step 1: Preparation of tert-butyl (4-((7-bromo-2-chloro-3-nitroquinolin-4-yl)amino)butyl)carbamate

To a solution of 7-bromo-2,4-dichloro-3-nitroquinoline (3.5 g, 10.9 mmol) in DCM was added tert-butyl N-(4-aminobutyl) carbamate (2.26 g, 11.9 mmol) and TEA (1.65 g, 16.3 mmol) dropwise at RT. The reaction mixture was stirred at 40° C. for 16 hours. LCMS showed the reaction was completed. The resulting mixture was diluted with water (50 mL) and extracted with DCM (50 mL×3). The combine organic phases were washed with brine, dried over Na₂SO₄, concentrated to give the crude. The residue was purified by silica gel column chromatography (PE:EA 5:1) to give tert-butyl (4-((7-bromo-2-chloro-3-nitroquinolin-4-yl) amino) butyl)carbamate (4.9 g, 85.3% yield) as a white solid.

LCMS (ESI) calcd for C₁₈H₂₂BrClN₄O₄[M+H]⁺ m/z=473.1, found 473.

### Step 2: Preparation of tert-butyl (4-((3-amino-7-bromo-2-chloroquinolin-4-yl)amino)butyl)carbamate

To a solution of tert-butyl (4-((7-bromo-2-chloro-3-nitroquinolin-4-yl)amino)butyl)carbamate (4.9 g, 10.3 mmol) in AcOH was added Fe (5.8 g, 103 mmol). The reaction mixture was stirred at RT for 10 hours. LCMS showed the reaction was completed. It was diluted with DCM and the solution was filtered through celite and the filtrate was concentrated under vacuum to give the crude. The residue was purified by silica gel column chromatography (PE:EA 2:1) to give the title compound (3.3 g, 65.1% yield) as a white solid.

LCMS (ESI) calcd for C₁₈H₂₄BrClN₄O₂[M+H]⁺ m/z=443.1, found 443.

### Step 3: tert-butyl N-(4-{[7-bromo-2-chloro-3-(2-ethoxyacetamido)quinolin-4-yl]amino}butyl)carbamate

To a solution of tert-butyl (4-((3-amino-7-bromo-2-chloroquinolin-4-yl)amino)butyl)carbamate (3.3 g, 7.4 mmol) in DCM was added TEA (2.25 g, 22.2 mmol) and 2-ethoxyacetyl chloride (1.36 g, 11.1 mmol) at 0° C. The reaction mixture was warmed to RT for 16 h. LCMS showed the reaction was completed. The reaction was diluted with water and extracted with DCM (50 mL×3). The combined organic phases were washed with brine, dried over Na₂SO₄, filtered and concentrated in vacuo. The residue was purified by silica gel column chromatography (PE:EA 2:1) to give the title compound (1.69 g, 39.1% yield) as a white solid.

LCMS (ESI) calcd for C₂₂H₃₀BrClN₄O₄[M+H]⁺ m/z=529.1, found 529.

### Step 4: tert-butyl N-{4-[4-amino-7-bromo-2-(ethoxymethyl)imidazo[4,5-c]quinolin-1-yl]butyl)carbamate

A solution of tert-butyl N-(4-{[7-bromo-2-chloro-3-(2-ethoxyacetamido)quinolin-4-yl]amino}butyl)carbamate (1.69 g, 3.2 mmol) in NH₃-MeOH (20 mL) was stirred at 160° C. for 8 hours in a high-pressure reactor. After cooling to ambient temperature, the mixture was concentrated in vacuo. The residue was purified by silica gel column chromatography (PE:EA 2:1) to afford the title compound (0.9 g, 50% yield) as a yellow solid.

LCMS (ESI) calcd for C₂₂H₃₀BrN₅O₃[M+H]⁺ m/z=492.1, found 492.

### Step 5: tert-butyl N-{4-[4-amino-7-benzyl-2-(ethoxymethyl)imidazo[4,5-c]quinolin-1-yl]butyl}carbamate

To a solution of tert-butyl N-{4-[4-amino-7-bromo-2-(ethoxymethyl)imidazo[4,5-c]quinolin-1-yl]butyl}carbamate (160 mg, 0.32 mmol), Pd(PPh₃)Cl₂ (54 mg, 0.06 mmol) and Copper(I) iodide (138 mg, 0.65 mmol) in DMF stirred under nitrogen at RT was added a solution of benzyl(bromo)zinc (1.5 mL, 2M) dropwise. The reaction mixture was stirred at 50° C. for 3 hours. It was concentrated and diluted with EA. The organic phase was washed with brine, dried over Na₂SO₄, filtered, and concentrated in vacuo. The residue was purified by silica gel column chromatography (PE:EA 2:1) to afford the title compound (50 mg, 27.5% yield) as a yellow solid.

LCMS (ESI) calcd for C₂₉H₃₇N₅O₃ [M+H]⁺ m/z=504.2, found 504.

### Step 6: 1-(4-aminobutyl)-7-benzyl-2-(ethoxymethyl)-1H-imidazo[4,5-c]quinolin-4-amine (Compound 39)

To a solution of tert-butyl N-{4-[4-amino-7-benzyl-2-(ethoxymethyl)imidazo[4,5-c]quinolin-1-yl]butyl}carbamate (50 mg, 0.10 mmol) in DCM (5 mL) was added TFA (1.5 mL) dropwise at room temperature. The reaction mixture was stirred at room temperature for 3 hours. The solution was removed under reduced pressure to afford the crude product. The residue was purified by Prep-HPLC (column: Gemini-C18 150×21.2 mm, 5 um; mobile phase: ACN-H₂O (0.1% FA), gradient: 10-50) to afford Compound 39 (0.87 mg, 2% yield) as white solid.

LCMS (ESI) calcd for C₂₄H₂₉N₅O [M+H]⁺ m/z=404.2, found 404.

1H NMR (400 MHz, DMSO-d₆) δ 7.94 (d, J=8.3 Hz, 1H), 7.46 (s, 1H), 7.30 (d, J=4.3 Hz, 4H), 7.24-7.16 (m, 1H), 7.12 (d, J=8.8 Hz, 1H), 6.57 (s, 2H), 4.76 (s, 2H), 4.56 (s, 2H), 4.06 (s, 4H), 3.55 (d, J=7.0 Hz, 2H), 1.87 (s, 2H), 1.66 (s, 2H), 1.23 (s, 2H), 1.16 (t, J=6.9 Hz, 3H).

### Example 21

### Synthesis of Compound 40

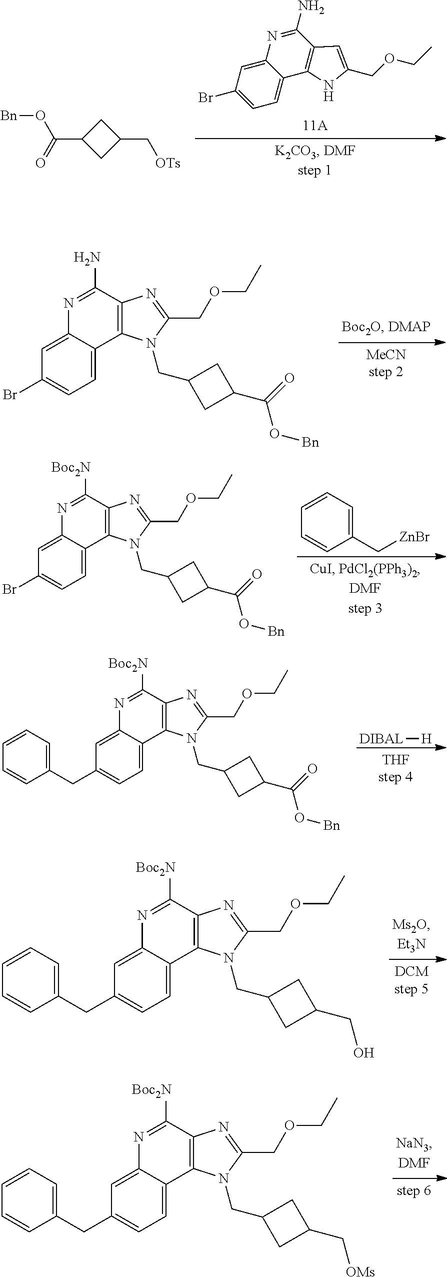

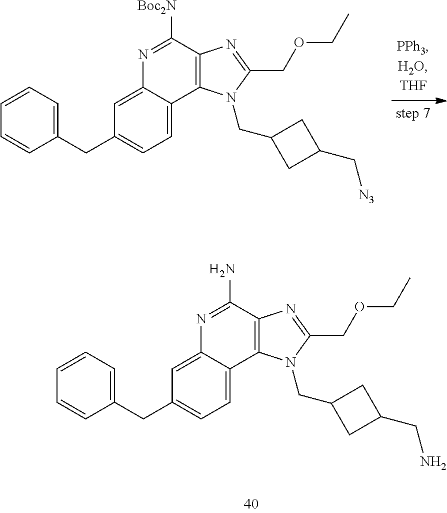

### Step 1: Preparation of benzyl 3-((4-amino-7-bromo-2-(ethoxymethyl)-1H-imidazo[4,5-c]quinolin-1-yl)methyl)cyclobutane-1-carboxylate

A mixture of 7-bromo-2-(ethoxymethyl)-1H-imidazo[4,5-c]quinolin-4-amine (800 mg, 2.49 mmol), benzyl 3-((tosyloxy)methyl)cyclobutane-1-carboxylate (1.87 g, 4.98 mmol) and K₂CO₃ (1.03 g, 7.47 mmol) in DMF (10 mL) was heated 80° C. for 12 hours. LCMS showed the reaction was completed. The resulting mixture was diluted with water (20 mL) and extracted with EtOAc (15 mL×3). The organic phases were washed with brine, dried over Na₂SO₄, concentrated and purified by column chromatography on silica gel (PE/EtOAc=1/4) to give benzyl 3-((4-amino-7-bromo-2-(ethoxymethyl)-1H-imidazo[4,5-c]quinolin-1-yl)methyl)cyclobutane-1-carboxylate (800 mg, 47.1%) as a yellow solid.

LCMS (ESI) calcd for C₂₆H₂₇BrN₄O₃[M+H]⁺ m/z=523.1, found 523.0.

### Step 2: Preparation of benzyl 3-((4-(bis(tert-butoxycarbonyl)amino)-7-bromo-2-(ethoxymethyl)-1H-imidazo[4,5-c]quinolin-1-yl)methyl)cyclobutane-1-carboxylate

To a solution of benzyl 3-((4-amino-7-bromo-2-(ethoxymethyl)-1H-imidazo[4,5-c]quinolin-1-yl)methyl)cyclobutane-1-carboxylate (800 mg, 1.52 mmol) and DMAP (74.55 mg, 0.61 mmol) in MeCN (15 mL) was added Boc₂O (1.66 g, 7.63 mmol) at room temperature. The mixture was heated at 75° C. for 4 hours. The resulting mixture was diluted with water (50 mL) and extracted with EA (50 mL×3). The organic phases were washed with brine, dried over Na₂SO₄, concentrated and purified by column chromatography on silica gel (PE/EtOAc=2/1) to give benzyl 3-((4-(bis(tert-butoxycarbonyl)amino)-7-bromo-2-(ethoxymethyl)-1H-imidazo[4,5-c]quinolin-1-yl)methyl)cyclobutane-1-carboxylate (800 mg, 72.6% yield) as a yellow solid.

LCMS (ESI) calcd for C₃₆H₄₃BrN₄O₇[M+H]⁺ m/z=723.2, found 723.0.

### Step 3: Preparation of benzyl 3-((7-benzyl-4-(bis(tert-butoxycarbonyl)amino)-2-(ethoxymethyl)-1H-imidazo[4,5-c]quinolin-1-yl)methyl)cyclobutane-1-carboxylate

To a stirred solution of benzyl 3-((4-(bis(tert-butoxycarbonyl)amino)-7-bromo-2-(ethoxymethyl)-1H-imidazo[4,5-c]quinolin-1-yl)methyl)cyclobutane-1-carboxylate (800 mg, 1.53 mmol), CuI (116.21 mg, 0.61 mmol) and Pd (PPh₃)₂Cl₂ (214.15 mg, 0.31 mmol) in DMF (3 mL) was added a solution of benzyl(bromo)zinc (432.83 mg, 1.83 mmol) in DMF (8 mL) dropwise at room temperature under an atmosphere of N₂. The reaction mixture was heated at 50° C. for 2 hours. LCMS showed the reaction was completed. The resulting mixture was diluted with water (20 mL) and extracted with EtOAc (20 mL×3). The organic phases were washed with brine, dried over Na₂SO₄, concentrated, and purified by column chromatography on silica gel (PE/EtOAc=1/1) to give benzyl 3-((7-benzyl-4-(bis(tert-butoxycarbonyl)amino)-2-(ethoxymethyl)-1H-imidazo[4,5-c]quinolin-1-yl)methyl)cyclobutane-1-carboxylate (800 mg, 71.2% yield) as a yellow solid.

LCMS (ESI) calcd for C₄₃H₅₀N₄O₇[M+H]⁺ m/z=735.4, found 735.0.

### Step 4: Preparation of di-tert-butyl (7-benzyl-2-(ethoxymethyl)-1-((3-(hydroxymethyl)cyclobutyl)methyl)-1H-imidazo[4,5-c]quinolin-4-yl)iminodicarbonate

To a stirred solution of benzyl 3-((7-benzyl-4-(bis(tert-butoxycarbonyl)amino)-2-(ethoxymethyl)-1H-imidazo[4,5-c]quinolin-1-yl)methyl)cyclobutane-1-carboxylate (200 mg, 0.27 mmol) in THF (3 mL) was added DIBAL-H (1M in hexane, 0.41 mL, 0.41 mmol) dropwise at 0° C. under an atmosphere of N₂. The reaction mixture was stirred at room temperature for 30 minutes. The resulting mixture was quenched with water (5 mL) and extracted with EtOAc (10 mL×3). The organic phases were washed with brine, dried over Na₂SO₄, concentrated and purified by column chromatography on silica gel (PE/EtOAc=1/4) to give di-tert-butyl (7-benzyl-2-(ethoxymethyl)-1-((3-(hydroxymethyl)cyclobutyl)methyl)-1H-imidazo[4,5-c]quinolin-4-yl)iminodicarbonate (150 mg, 88.1% yield) as a color oil.

LCMS (ESI) calcd for C₃₆H₄₆N₄O₆ [M+H]⁺ m/z=631.3, found 631.0.

### Step 5: Preparation of (3-((7-benzyl-4-(bis(tert-butoxycarbonyl)amino)-2-(ethoxymethyl)-1H-imidazo[4,5-c]quinolin-1-yl)methyl)cyclobutyl)methyl methanesulfonate

To a stirred solution of di-tert-butyl (7-benzyl-2-(ethoxymethyl)-1-((3-(hydroxymethyl)cyclobutyl)methyl)-1H-imidazo[4,5-c]quinolin-4-yl)iminodicarbonate (150 mg, 0.24 mmol) and Et₃N (72.19 mg, 0.71 mmol) in DCM (4 mL) was added methanesulfonic anhydride (124.27 mg, 0.71 mmol) in DCM (4 mL) dropwise. The reaction mixture was stirred at room temperature for 2 hours. LCMS showed the reaction was completed. The resulting mixture was diluted with water (10 mL) and extracted with DCM (15 mL×3). The organic phases were washed with brine, dried over Na₂SO₄, concentrated to give the crude (3-((7-benzyl-4-(bis(tert-butoxycarbonyl)amino)-2-(ethoxymethyl)-1H-imidazo[4,5-c]quinolin-1-yl)methyl)cyclobutyl)methyl methanesulfonate (100 mg) which was used in next reaction without further purification.

LCMS (ESI) calcd for C₃₇H₄₈N₄O₈S [M+H]⁺ m/z=709.3, found 709.0.

### Step 6: Preparation of di-tert-butyl (1-((3-(azidomethyl)cyclobutyl)methyl)-7-benzyl-2-(ethoxymethyl)-1H-imidazo[4,5-c]quinolin-4-yl)iminodicarbonate

To a stirred solution of (3-((7-benzyl-4-(bis(tert-butoxycarbonyl)amino)-2-(ethoxymethyl)-1H-imidazo[4,5-c]quinolin-1-yl)methyl)cyclobutyl)methyl methanesulfonate (100 mg, 0.14 mmol) in DMF (5 mL) was added NaN₃ (13.76 mg, 0.21 mmol) at room temperature. The reaction mixture was heated at 80° C. for 2 hours. LCMS showed the reaction was completed. The resulting mixture was diluted with water (5 mL) and extracted with EA (15 mL×3). The organic phases were washed with brine, dried over Na₂SO₄, concentrated to give crude di-tert-butyl (1-((3-(azidomethyl)cyclobutyl)methyl)-7-benzyl-2-(ethoxymethyl)-1H-imidazo[4,5-c]quinolin-4-yl)iminodicarbonate (60 mg) which was used in next reaction without further purification.

LCMS (ESI) calcd for C₃₆H₄₅N₇O₅ [M+H]⁺ m/z=656.3, found 656.0.

### Step 7: Preparation of 1-((3-(aminomethyl)cyclobutyl)methyl)-7-benzyl-2-(ethoxymethyl)-1H-imidazo[4,5-c]quinolin-4-amine (Compound 40)

To a solution of di-tert-butyl (1-((3-(azidomethyl)cyclobutyl)methyl)-7-benzyl-2-(ethoxymethyl)-1H-imidazo[4,5-c]quinolin-4-yl)iminodicarbonate (60 mg, 0.09 mmol) in THF/H₂O (5:1, 6 mL) was added triphenylphosphine (36 mg, 0.14 mmol) in one charge. The reaction mixture was stirred at 25° C. for 12 hours. LCMS showed the reaction was completed. The reaction mixture was diluted with water (10 mL) and the pH was adjusted to 2.5-3.5 with 5N HCl aqueous solution. The aqueous layer was washed with EtOAc (10 mL×3). The separated aqueous layer was concentrated and purified by prep-HPLC (Column: Gemini-C₁₈,150×21.2 mm, 5 um; Mobile Phase: ACN-H₂O (0.1% FA), Gradient: 20%-65%) to give 1-((3-(aminomethyl)cyclobutyl)methyl)-7-benzyl-2-(ethoxymethyl)-1H-imidazo[4,5-c]quinolin-4-amine (Compound 40, 2 mg, 5.2% yield) as a white solid.

LCMS (ESI) calcd for C₂₆H₃₁N₅O [M+H]⁺ m/z=430.2, found 430.0.

### Example 22

### Synthesis of Compound 41

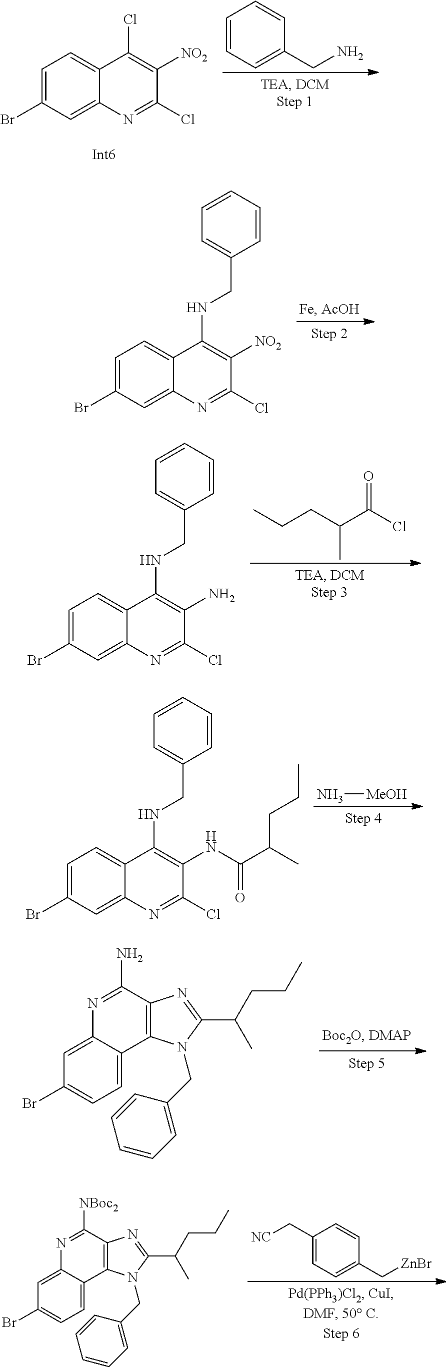

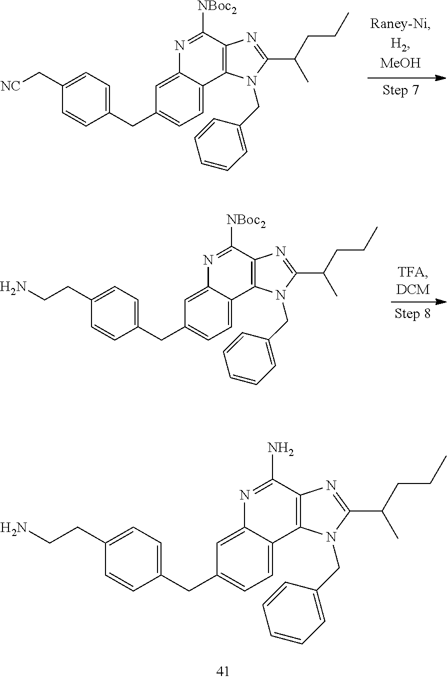

### Step 1: Preparation of N-benzyl-7-bromo-2-chloro-3-nitroquinolin-4-amine

To a solution of 7-bromo-2,4-dichloro-3-nitroquinoline (3 g, 9.32 mmol) and Et₃N (1.41 g, 13.95 mmol) in DCM (30 mL) was added phenylmethanamine (1 g, 9.32 mmol) dropwise at 0° C. Then the mixture was stirred at RT for 3 hours. LCMS showed the reaction was completed. The resulting mixture was diluted with water (50 mL) and extracted with DCM (50 mL×3). The combine organic phases were washed with brine, dried over Na₂SO₄, concentrated to give the crude. The residue was purified by combi-flash (PE:EA 1:2) to afford N-benzyl-7-bromo-2-chloro-3-nitroquinolin-4-amine (3.4 g, 92.95% yield) as yellow solid.

LCMS (ESI) calcd for C₁₆H₁₁BrClN₃O₂ [M+H]⁺ m/z=391.1, found 391.

### Step 2: N⁴-benzyl-7-bromo-2-chloroquinoline-3,4-diamine

To a solution of N-benzyl-7-bromo-2-chloro-3-nitroquinolin-4-amine (3.4 g, 8.66 mmol) in AcOH (40 mL) was added Fe (4.86 g, 86.60 mmol) at RT. The mixture was stirred at RT for 6 hours. LCMS showed the reaction was completed. The resulting mixture was filtrated and the filtrated was concentrated. The residue was diluted with water (80 mL) and extracted with EA (80 mL×3). The combine organic phases were washed with brine, dried over Na₂SO₄, concentrated to give the crude. The residue was purified by combi-flash (PE:EA 1:5) to afford N⁴-benzyl-7-bromo-2-chloroquinoline-3,4-diamine (2.8 g, 89.2% yield) as yellow solid.

LCMS (ESI) calcd for C₁₆H₁₃BrClN₃ [M+H]⁺ m/z=361.9, found 362.

### Step 3: N-(4-(benzylamino)-7-bromo-2-chloroquinolin-3-yl)-2-methylpentanamide

To a solution of N⁴-benzyl-7-bromo-2-chloroquinoline-3,4-diamine (1.5 g, 4.16 mmol) and Et₃N (0.63 g, 6.23 mmol) in DCM (20 mL) was added 2-methylpentanoyl chloride (0.61 g, 4.57 mmol) dropwise at 0° C. Then the mixture was stirred at RT for 3 hours. LCMS showed the reaction was completed. The resulting mixture was diluted with water (30 mL) and extracted with DCM (30 mL×3). The combine organic phases were washed with brine, dried over Na₂SO₄, concentrated to give the crude. The residue was purified by combi-flash (PE:EA 1:2) to afford N-(4-(benzylamino)-7-bromo-2-chloroquinolin-3-yl)-2-methylpentanamide (1.25 g, 65.55% yield) as yellow solid.

LCMS (ESI) calcd for C₂₂H₂₃BrClN₃O [M+H]⁺ m/z=460.1, found 460.

### Step 4: Preparation of 1-benzyl-7-bromo-2-(pentan-2-yl)-1H-imidazo[4,5-c]quinolin-4-amine

A solution of N-(4-(benzylamino)-7-bromo-2-chloroquinolin-3-yl)-2-methylpentanamide (1.25 g, 2.71 mmol) in NH₃-MeOH (20 mL) was stirred at 160° C. for 8 hours in a high-pressure reactor. After cooling to ambient temperature, the resulting mixture was concentrated in vacuo. The residue was washed with PE:EA (1:1) to give 1-benzyl-7-bromo-2-(pentan-2-yl)-1H-imidazo[4,5-c]quinolin-4-amine (1.1 g, 97.32% yield) as yellow solid.

LCMS (ESI) calcd for C₂₂H₂₃BrN₄ [M+H]⁺ m/z=423.1, found 423.

### Step 5: Preparation of di-tert-butyl (1-benzyl-7-bromo-2-(pentan-2-yl)-1H-imidazo[4,5-c]quinolin-4-yl)iminodicarbonate

To a solution of 1-benzyl-7-bromo-2-(pentan-2-yl)-1H-imidazo[4,5-c]quinolin-4-amine (1.1 g, 2.60 mmol) and DMAP (0.13 g, 1.04 mmol) in MeCN (20 mL) was added Boc₂O (3.97 g, 18.188 mmol) at 75° C. The mixture was stirred at 75° C. for 4 hours. The resulting mixture was diluted with water (30 mL) and extracted with EA (40 mL×3). The combine organic phases were washed with brine, dried over Na₂SO₄, concentrated to give the crude. The residue was purified by combi-flash (PE:EA 2:1) to afford di-tert-butyl (1-benzyl-7-bromo-2-(pentan-2-yl)-1H-imidazo[4,5-c]quinolin-4-yl)iminodicarbonate (1.1 g, 67.9% yield) as yellow solid.

LCMS (ESI) calcd for C₃₂H₃₉BrN₄O₄[M+H]⁺ m/z=623.2, found 623.

### Step 6: Preparation of di-tert-butyl (1-benzyl-7-(4-(cyanomethyl)benzyl)-2-(pentan-2-yl)-1H-imidazo[4,5-c]quinolin-4-yl)iminodicarbonate

To a solution of di-tert-butyl(1-benzyl-7-bromo-2-(pentan-2-yl)-1H-imidazo[4,5-c]quinolin-4-yl) iminodicarbonate (1.1 g, 1.76 mmol), Pd(PPh₃)Cl₂ (0.124 g, 0.18 mmol) and CuI (67 mg, 0.35 mmol) in DMF (10 mL) was added a solution of (4-(cyanomethyl)benzyl)zinc(II) bromide in DMF (15 mL). The mixture was stirred at 50° C. for 1 hour. LCMS showed the reaction was completed. The resulting mixture was diluted with water (90 mL) and extracted with EA (50 mL×3). The combine organic phases were washed with brine, dried over Na₂SO₄, concentrated to give the crude. The residue was purified by combi-flash (PE:EA 1:2) to afford di-tert-butyl (1-benzyl-7-(4-(cyanomethyl)benzyl)-2-(pentan-2-yl)-1H-imidazo[4,5-c]quinolin-4-yl)iminodi carbonate (0.9 g, 75.72% yield) as yellow solid.

LCMS (ESI) calcd for C₄₁H₄₇N₅O₄ [M+H]⁺ m/z=674.3, found 674.

### Step 7: Preparation of di-tert-butyl (7-(4-(2-aminoethyl)benzyl)-1-benzyl-2-(pentan-2-yl)-1H-imidazo[4,5-c]quinolin-4-yl)iminodicarbonate

To a solution of di-tert-butyl (1-benzyl-7-(4-(cyanomethyl)benzyl)-2-(pentan-2-yl)-1H-imidazo[4,5-c]quinolin-4-yl)iminodicarbonate (900 mg, 1.34 mmol) in NH₃-MeOH (10 mL) was added the right amount of Raney-Ni washed with MeOH. The mixture was stirred at RT for 2 hours under H₂. LCMS showed the reaction was completed. Then the mixture was filtered through celite and concentrated to give the crude without further purification and used in the next step.

LCMS (ESI) calcd for C₄₁H₅₁N₅O₄ [M+H]⁺ m/z=678.4, found 678.

### Step 8: Preparation of 7-(4-(2-aminoethyl)benzyl)-1-benzyl-2-(pentan-2-yl)-1H-imidazo[4,5-c]quinolin-4-amine (Compound 41)

To a solution of di-tert-butyl (7-(4-(2-aminoethyl)benzyl)-1-benzyl-2-(pentan-2-yl)-1H-imidazo[4,5-c]quinolin-4-yl)iminodicarbonate (100 mg, 0.15 mmol) in DCM (6 mL) was added TFA (2 mL) dropwise at 0° C. The mixture was stirred at RT for 2 hours. LCMS showed the reaction was completed. The reaction was concentrated and purified by Prep-HPLC (Column: Gemini-C18,150×21.2 mm, 5 um; Mobile Phase: ACN-H₂O (0.05% NH₃), Gradient: 10-40) to give Compound 41 (5.94 mg, 8.43% yield) as white solid.

LCMS (ESI) calcd for C₃₁H₃₅N₅[M+H]⁺ m/z=478.3, found 478.

¹H NMR (400 MHz, DMSO-d₆) δ 8.55 (s, 1H), 7.84 (d, J=8.5 Hz, 2H), 7.55 (s, 1H), 7.37-7.22 (m, 3H), 7.20 (q, J=8.2 Hz, 4H), 7.04 (d, J=7.4 Hz, 2H), 5.95 (s, 2H), 4.03 (s, 2H), 3.26 (m, 2H), 3.07-2.92 (m, 2H), 2.87-2.72 (m, 2H), 1.88-1.51 (m, 2H), 1.33-1.10 (m, 5H), 0.75 (t, J=7.3 Hz, 3H).

### Example 23

### Synthesis of Compound 42

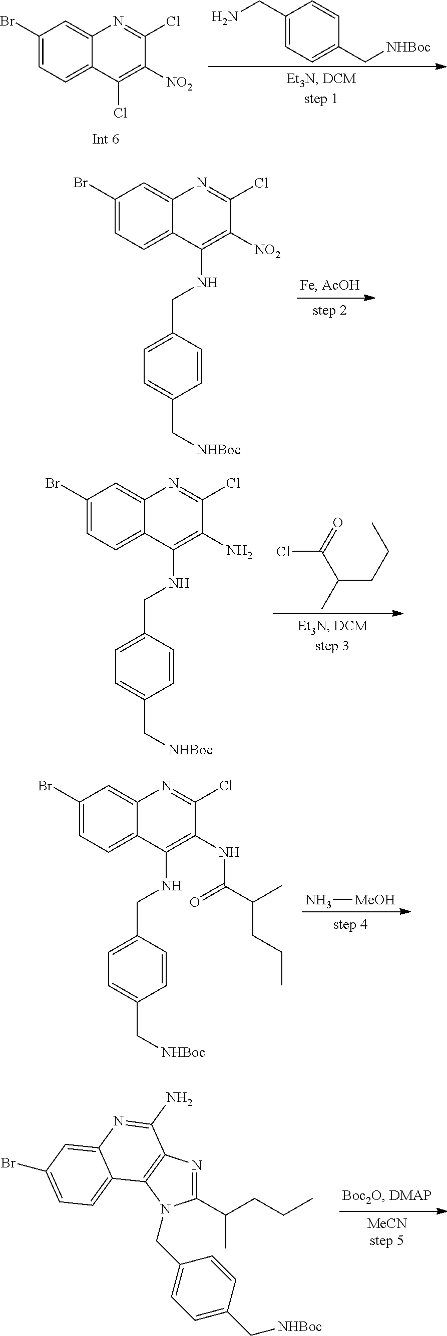

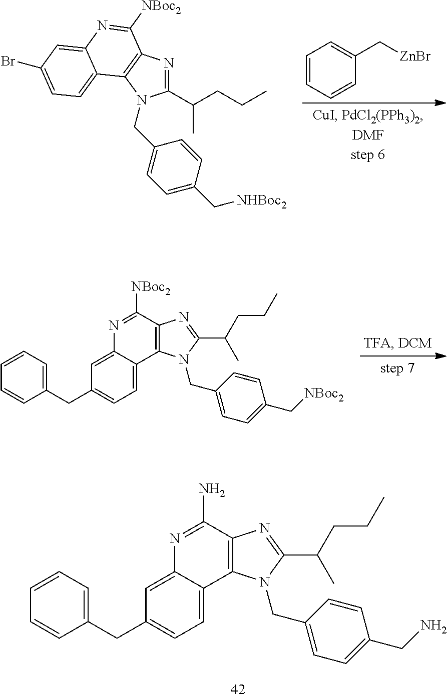

### Step 1: Preparation of tert-butyl (4-(((7-bromo-2-chloro-3-nitroquinolin-4-yl)amino)methyl)benzyl)carbamate

To a solution of 7-bromo-2,4-dichloro-3-nitroquinoline (2 g, 6.2 mmol) and Et₃N (1.88 g, 18.6 mmol) in DCM (40 mL) was added tert-butyl (4-(aminomethyl)benzyl)carbamate (1.61 g, 6.82 mmol) dropwise at 0° C. The mixture was stirred room temperature for 4 hours. LCMS showed the reaction was completed. The resulting mixture was diluted with water (20 mL) and extracted with DCM (20 mL×3). The organic phases were washed with water and brine, dried over Na₂SO₄ and concentrated to give the crude tert-butyl (4-(((7-bromo-2-chloro-3-nitroquinolin-4-yl)amino)methyl)benzyl)carbamate (2 g) which was used in the next reaction without further purification.

LCMS (ESI) calcd for C₂₂H₂₂BrClN₄O₄[M+H]⁺ m/z=521.1, found 521.0.

### Step 2: Preparation of tert-butyl (4-(((3-amino-7-bromo-2-chloroquinolin-4-yl)amino)methyl)benzyl)carbamate

To a stirred suspension of tert-butyl (4-(((7-bromo-2-chloro-3-nitroquinolin-4-yl)amino)methyl)benzyl)carbamate (2 g, 3.8 mmol) in AcOH (40 mL) was added Fe (2.12 g, 38 mmol) at room temperature. The reaction mixture was stirred at room temperature for 6 hours. LCMS showed the reaction was completed. The resulting mixture was filtered through celite and the filtrate was concentrated in vacuo. The residue was diluted with water (50 mL) and extracted with EtOAc (50 mL×3). The organic phases were washed with brine, dried over Na₂SO₄, concentrated and purified by column chromatography on silica gel (PE/EtOAc=1/2) to give tert-butyl (4-(((3-amino-7-bromo-2-chloroquinolin-4-yl)amino)methyl)benzyl)carbamate (1.5 g, 80.3% yield) as a green solid.

LCMS (ESI) calcd for C₂₂H₂₄BrClN₄O₂[M+H]⁺ m/z=491.1, found 491.0.

### Step 3: Preparation of tert-butyl (4-(((7-bromo-2-chloro-3-(2-methylpentanamido)quinolin-4-yl)amino)methyl)benzyl)carbamate

To a stirred solution of tert-butyl (4-(((3-amino-7-bromo-2-chloroquinolin-4-yl)amino)methyl)benzyl)carbamate (1.5 g, 3.05 mmol) in DCM (30 mL) at 0° C. was added 2-methylpentanoyl chloride (0.49 g, 3.66 mmol) dropwise. The reaction mixture was stirred at room temperature for 3 hours. LCMS showed the reaction was completed. The resulting mixture was diluted with water (20 mL) and extracted with DCM (20 mL×3). The organic phases were washed with brine, dried over Na₂SO₄, concentrated and purified by flash column chromatography on silica gel (PE/EtOAc=1/4) to give tert-butyl (4-(((7-bromo-2-chloro-3-(2-methylpentanamido)quinolin-4-yl)amino)methyl)benzyl)carbamate (1.3 g, 72.2% yield) as a yellow solid.

LCMS (ESI) calcd for C₂₈H₃₄BrClN₄O₃[M+H]⁺ m/z=589.2, found 589.0.

### Step 4: Preparation of tert-butyl (4-((4-amino-7-bromo-2-(pentan-2-yl)-1H-imidazo[4,5-c]quinolin-1-yl)methyl)benzyl)carbamate

A solution of tert-butyl (4-(((7-bromo-2-chloro-3-(2-methylpentanamido)quinolin-4-yl)amino)methyl)benzyl)carbamate (1.3 g, 2.2 mmol) in 7M NH₃-MeOH (15 mL) was heated at 160° C. for 8 hours in a high-pressure reactor. After cooling to ambient temperature, the reaction mixture was concentrated to give the crude tert-butyl (4-((4-amino-7-bromo-2-(pentan-2-yl)-1H-imidazo[4,5-c]quinolin-1-yl)methyl)benzyl)carbamate (1 g) as yellow solid which was used in next reaction without further purification.

LCMS (ESI) calcd for C₂₈H₃₄BrN₅O₂[M+H]⁺ m/z=552.2, found 552.0.

### Step 5: Preparation of di-tert-butyl (1-(4-((bis(tert-butoxycarbonyl)amino)methyl)benzyl)-7-bromo-2-(pentan-2-yl)-1H-imidazo[4,5-c]quinolin-4-yl)iminodicarbonate

To a solution of tert-butyl (4-((4-amino-7-bromo-2-(pentan-2-yl)-1H-imidazo[4,5-c]quinolin-1-yl)methyl)benzyl)carbamate (1 g, 1.8 mmol) and DMAP (90 mg, 0.72 mmol) in MeCN (20 mL) was added Boc₂O (1.96 g, 9 mmol) at room temperature. The mixture was heated at 75° C. for 2 hours. The resulting mixture was diluted with water (20 mL) and extracted with EtOAc (50 mL×3). The organic phases were washed with brine, dried over Na₂SO₄, concentrated, and purified by column chromatography on silica gel (PE/EtOAc=1/1) to give di-tert-butyl (1-(4-((bis(tert-butoxycarbonyl)amino)methyl)benzyl)-7-bromo-2-(pentan-2-yl)-1H-imidazo[4,5-c]quinolin-4-yl)iminodicarbonate (0.8 g, 70.5% yield) as a yellow solid.

LCMS (ESI) calcd for C₄₃H₅₈BrN₅O₈ [M+H]⁺ m/z=852.3, found 852.0.

### Step 6: Preparation of di-tert-butyl (7-benzyl-1-(4-((bis(tert-butoxycarbonyl)amino)methyl)benzyl)-2-(pentan-2-yl)-1H-imidazo[4,5-c]quinolin-4-yl)iminodicarbonate

To a solution of di-tert-butyl (1-(4-((bis(tert-butoxycarbonyl)amino)methyl)benzyl)-7-bromo-2-(pentan-2-yl)-1H-imidazo[4,5-c]quinolin-4-yl)iminodicarbonate (800 mg, 0.94 mmol), CuI (17.86 mg, 0.09 mmol) and Pd(PPh₃)₂Cl₂ (65.84 mg, 0.09 mmol) in DMF (3 mL) was added a solution of benzylzinc(II) bromide (443.57 mg, 1.88 mmol) in DMF (5 mL). The mixture was heated at 50′C for 2 hours. LCMS showed the reaction was completed. The resulting mixture was diluted with water (20 mL) and extracted with EA (15 mL×3). The organic phases were washed with brine, dried over Na₂SO₄, concentrated purified by column chromatography on silica gel (PE/EA=1/2) to give di-tert-butyl (7-benzyl-1-(4-((bis(tert-butoxycarbonyl)amino)methyl)benzyl)-2-(pentan-2-yl)-1H-imidazo[4,5-c]quinolin-4-yl)iminodicarbonate (700 mg, 86.2% yield) as yellow solid.

LCMS (ESI) calcd for C₅H₆₅N₅O₈ [M+H]⁺ m/z=864.4, found 864.0.

### Step 7: Preparation of 1-(4-(aminomethyl)benzyl)-7-benzyl-2-(pentan-2-yl)-1H-imidazo[4,5-c]quinolin-4-amine (Compound 42)

To a solution of di-tert-butyl (7-benzyl-1-(4-((bis(tert-butoxycarbonyl)amino)methyl)benzyl)-2-(pentan-2-yl)-1H-imidazo[4,5-c]quinolin-4-yl)iminodicarbonate (400 mg, 0.71 mmol) in DCM (4 mL) was added TFA (2 mL) dropwise at 0° C. The mixture was stirred at room temperature for 2 hours. The resulting mixture was concentrated in vacuo. The residue was purified by Prep-HPLC (Column: Gemini-Cis, 150×21.2 mm, 5 um; Mobile Phase: ACN-H₂O (0.1% FA), Gradient: 10%-60%) to give 1-(4-(aminomethyl)benzyl)-7-benzyl-2-(pentan-2-yl)-1H-imidazo[4,5-c]quinolin-4-amine (160 mg, 48.6% yield) as a white solid.

LCMS (ESI) calcd for C₃₀H₃₃N₅[M+H]⁺ m/z=464.3, found 464.0.

¹H NMR (400 MHz, DMSO-d₆, ppm) δ 8.14 (s, 1H), 8.05 (s, 2H), 7.72 (d, J=8.5 Hz, 1H), 7.49 (s, 1H), 7.38 (d, J=8.2 Hz, 2H), 7.32-7.15 (m, 5H), 7.05 (d, J=8.2 Hz, 3H), 5.93 (s, 2H), 3.99 (d, J=15.9 Hz, 4H), 3.20 (dd, J=13.7, 6.8 Hz, 1H), 1.89-1.80 (m, 1H), 1.60-1.55 (m, 1H), 1.28-1.23 (m, 5H), 0.78 (t, J=7.3 Hz, 3H).

Compound 43 was prepared by similar method as described in Example 22 using corresponding reagents.

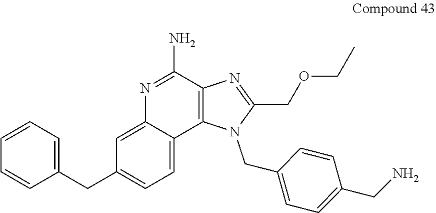 [SMILES: CCOCc1nc2c(N)nc3cc(Cc4ccccc4)ccc3c2n1Cc1ccc(CN)cc1]

LCMS (ESI) calcd for C₂₈H₂₉N₅O [M+H]⁺ m/z=452.2, found 452.

¹H NMR (400 MHz, DMSO-d₆, ppm) δ 8.15 (s, 1H), 7.66 (d, J=8.4 Hz, 1H), 7.42 (d, J=1.3 Hz, 1H), 7.36 (d, J=8.2 Hz, 2H), 7.32-7.22 (m, 4H), 7.17 (ddd, J=8.6, 4.2, 1.9 Hz, 1H), 7.10 (d, J=8.2 Hz, 2H), 6.89 (dd, J=8.4, 1.5 Hz, 1H), 6.75 (s, 2H), 5.89 (s, 2H), 4.77 (s, 2H), 3.96 (d, J=4.6 Hz, 4H), 3.48 (q, J=7.0 Hz, 2H), 0.97 (t, J=7.0 Hz, 3H).

### Example 24

### Synthesis of Compound 45

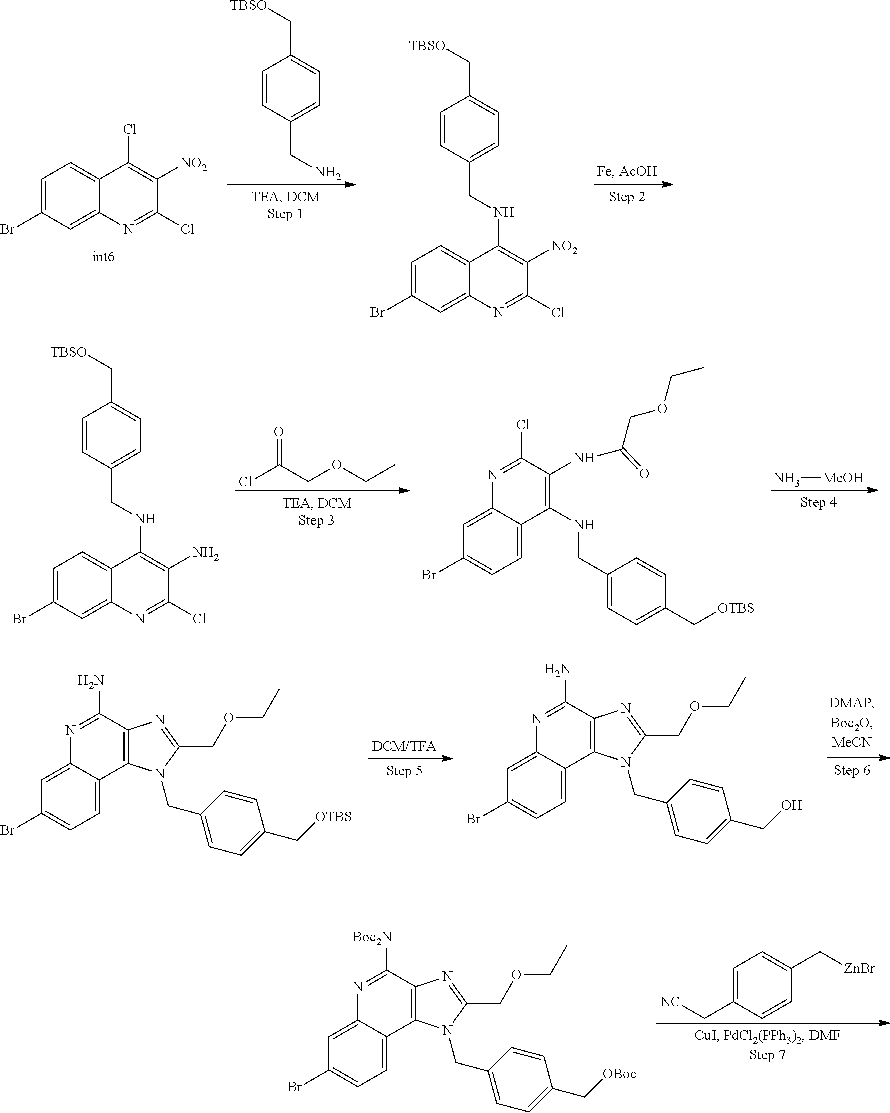

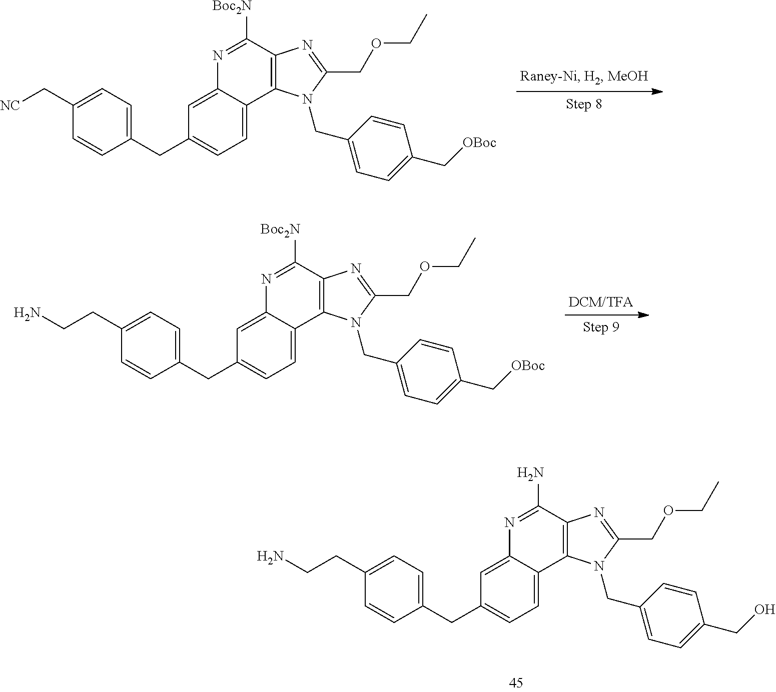

### Step 1: Preparation of 7-bromo-N-(4-(((tert-butyldimethylsilyl)oxy)methyl)benzyl)-2-chloro-3-nitroquinolin-4-amine

To a solution of 7-bromo-2,4-dichloro-3-nitroquinoline (2 g, 6.25 mmol) and Et₃N (0.95 g, 9.375 mmol) in DCM (20 mL) was added (4-(((tert-butyldimethylsilyl)oxy)methyl)phenyl)methanamine (1.73 g, 6.88 mmol) dropwise at 0′C. Then the mixture was stirred at RT for 3 hours. LCMS showed the reaction was completed. The resulting mixture was diluted with water (50 mL) and extracted with DCM (50 mL×3). The organic phases were washed with brine, dried over Na₂SO₄, concentrated to give the crude title compound. The residue was purified by combi-flash (PE:EA=1:1) to afford 7-bromo-N-(4-(((tert-butyldimethylsilyl)oxy)methyl)benzyl)-2-chloro-3-nitroquinolin-4-amine (2.2 g, 65.87% yield) as yellow solid.

LCMS (ESI) calcd for C₂₃H₂₇BrClN₃O₃Si [M+H]⁺ m/z=536.1, found 536.

### Step 2: 7-bromo-N4-(4-(((tert-butyldimethylsilyl)oxy)methyl)benzyl)-2-chloroquinoline-3,4-diamine

To a solution of 7-bromo-N-(4-(((tert-butyldimethylsilyl)oxy)methyl)benzyl)-2-chloro-3-nitroquinolin-4-amine (2.2 g, 4.10 mmol) in AcOH (30 mL) was added Fe (2.3 g, 41.04 mmol) at RT. The mixture was stirred at RT for 6 hours. LCMS showed the reaction was completed. The resulting mixture was concentrated in vacuo. The residue was diluted with water (60 mL) and extracted with EA (60 mL×3). The organic phases were washed with brine, dried over Na₂SO₄, concentrated to give the crude title compound. The residue was purified by silica gel column chromatography (PE:EA 1:2) to afford 7-bromo-N4-(4-(((tert-butyldimethylsilyl)oxy) methyl) benzyl)-2-chloroquinoline-3,4-diamine (0.9 g, 43.48% yield) as yellow solid.

LCMS (ESI) calcd for C₂₃H₂₉BrClN₃OSi [M+H]⁺ m/z=506.1, found 506.

### Step 3: N-(7-bromo-4-((4-(((tert-butyldimethylsilyl)oxy)methyl)benzyl)amino)-2-chloroquinolin-3-yl)-2-ethoxyacetamide

To a solution of 7-bromo-N4-(4-(((tert-butyldimethylsilyl)oxy)methyl)benzyl)-2-chloroquinoline-3,4-diamine (900 mg, 1.78 mmol) and Et₃N (270 mg, 2.67 mmol) in DCM (20 mL) was added 2-ethoxyacetyl chloride (239 mg, 1.96 mmol) dropwise at 0° C. Then the mixture was stirred at RT for 3 hours. LCMS showed the reaction was completed. It was diluted with DCM and the solution was filtered through celite. The filtrate was concentrated under vacuum to give the crude. The residue was diluted with water (100 mL) and extracted with EA (60 mL×3). The combine organic phases were washed with brine, dried over Na₂SO₄, concentrated to give the crude. The residue was purified by silica gel column chromatography (PE:EA=1:1) to afford N-(7-bromo-4-((4-(((tert-butyldimethylsilyl) oxy) methyl) benzyl) amino)-2-chloroquinolin-3-yl)-2-ethoxyacetamide (960 mg, 91.17% yield) as yellow solid.

LCMS (ESI) calcd for C₂₇H₃₅BrClN₃O₃Si [M+H]⁺ m/z=592.1, found 592.

### Step 4: Preparation of 7-bromo-1-(4-(((tert-butyldimethylsilyl)oxy)methyl)benzyl)-2-(ethoxymethyl)-1H-imidazo[4,5-c]quinolin-4-amine

A solution of N-(7-bromo-4-((4-(((tert-butyldimethylsilyl)oxy)methyl)benzyl)amino)-2-chloroquinolin-3-yl)-2-ethoxyacetamide (960 mg, 1.62 mmol) in NH₃-MeOH (10 mL) was stirred at 160° C. for 8 hours in a high-pressure reactor. After cooling to ambient temperature, the resulting mixture was concentrated in vacuo. The residue was washed with PE:EA (1:1) to give 7-bromo-1-(4-(((tert-butyldimethylsilyl)oxy)methyl)benzyl)-2-(ethoxymethyl)-1H-imidazo[4,5-c]quinolin-4-amine (400 mg, 44.54% yield) as yellow solid.

LCMS (ESI) calcd for C₂₇H₃₅BrN₄O₂Si [M+H]⁺ m/z=555.2, found 555.

### Step 5: Preparation of (4-((4-amino-7-bromo-2-(ethoxymethyl)-1H-imidazo[4,5-c]quinolin-1-yl)methyl)phenyl)methanol

To a solution of 7-bromo-1-(4-(((tert-butyldimethylsilyl)oxy)methyl)benzyl)-2-(ethoxymethyl)-1H-imidazo[4,5-c]quinolin-4-amine (400 mg, 0.72 mmol) in DCM (2 mL) was added TFA (1 mL) dropwise at 0° C. The mixture was stirred at RT for 2 hours. LCMS showed the reaction was completed. The resulting mixture was concentrated in vacuo to afford crude (4-((4-amino-7-bromo-2-(ethoxymethyl)-1H-imidazo[4,5-c]quinolin-1-yl)methyl)phenyl)methanol (300 mg, 94.6% yield).

LCMS (ESI) calcd for C₂₁H₂₁BrN₄O₂ [M+H]⁺ m/z=441.1, found 441.

### Step 6: Preparation of di-tert-butyl (7-bromo-1-(4-(((tert-butoxycarbonyl)oxy)methyl)benzyl)-2-(ethoxymethyl)-1H-imidazo[4,5-c]quinolin-4-yl)iminodicarbonate

To a solution of (4-((4-amino-7-bromo-2-(ethoxymethyl)-1H-imidazo[4,5-c]quinolin-1-yl)methyl)phenyl)methanol (300 mg, 0.68 mmol) and DMAP (33 mg, 0.27 mmol) in MeCN (5 mL) was added Boc₂O (1040 mg, 4.76 mmol) at 75′C. The mixture was stirred at 75° C. for 4 hours. The resulting mixture was diluted with water (20 mL) and extracted with EA (30 mL×3). The combine organic phases were washed with brine, dried over Na₂SO₄, concentrated to give the crude. The residue was purified by silica gel column chromatography (PE:EA 2:1) to afford di-tert-butyl (7-bromo-1-(4-(((tert-butoxycarbonyl)oxy)methyl)benzyl)-2-(ethoxymethyl)-1H-imidazo[4,5-c]quinolin-4-yl)iminodicarbonate (200 mg, 39.68% yield) as yellow solid.

LCMS (ESI) calcd for C₃₆H₄₅BrN₄O₈ [M+H]⁺ m/z=741.2, found 741.

### Step 7: Preparation of di-tert-butyl (1-(4-(((tert-butoxycarbonyl) oxy) methyl) benzyl)-7-(4-(cyanomethyl) benzyl)-2-(ethoxymethyl)-1H-imidazo[4,5-c]quinolin-4-yl)iminodicarbonate

To a solution of di-tert-butyl (7-bromo-1-(4-(((tert-butoxycarbonyl)oxy)methyl)benzyl)-2-(ethoxymethyl)-1H-imidazo[4,5-c]quinolin-4-yl)iminodicarbonate (200 mg, 0.27 mmol), Pd(PPh₃)Cl₂(19 mg, 0.027 mmol) and CuI (10 mg, 0.054 mmol) in DMF (3 mL) was added a solution of (4-(cyanomethyl)benzyl)zinc(II) bromide in DMF (5 mL). The mixture was stirred at 50′C for 0.5 h. LCMS showed the reaction was completed. The resulting mixture was diluted with water (30 mL) and extracted with EA (20 mL×3). The combine organic phases were washed with brine, dried over Na₂SO₄, concentrated to give the crude. The residue was purified by silica gel column chromatography (PE:EA1:1) to afford di-tert-butyl (1-(4-(((tert-butoxycarbonyl) oxy) methyl)benzyl)-7-(4-(cyanomethyl)benzyl)-2-(ethoxymethyl)-1H-imidazo[4,5-c]quinolin-4-yl) iminodicarbonate (120 mg, 56.13% yield) as yellow solid.

LCMS (ESI) calcd for C₄₅H₅₃N₅O₈ [M+H]⁺ m/z=792.4, found 792.

### Step 8: Preparation of di-tert-butyl (7-(4-(2-aminoethyl) benzyl)-1-(4-(((tert-butoxycarbonyl) oxy) methyl) benzyl)-2-(ethoxymethyl)-1H-imidazo[4,5-c]quinolin-4-yl) iminodicarbonate

To a solution of di-tert-butyl (1-(4-(((tert-butoxycarbonyl) oxy) methyl)benzyl)-7-(4-(cyanomethyl)benzyl)-2-(ethoxymethyl)-1H-imidazo[4,5-c]quinolin-4-yl)iminodicarbonate (120 mg, 0.15 mmol) in NH₃-MeOH (5 mL) was added the right amount of Raney-Ni washed with MeOH. The mixture was stirred at RT for 2 hours under H₂. LCMS showed the reaction was completed. Then the mixture was filtered through celite and concentrated under vacuo to give crude di-tert-butyl (7-(4-(2-aminoethyl)benzyl)-1-(4-(((tert-butoxycarbonyl)oxy)methyl)benzyl)-2-(ethoxymethyl)-1H-imidazo[4,5-c]quinolin-4-yl)iminodicarbonate (100 mg, 84.8% yield) which was used directly in next step without further purification.

LCMS (ESI) calcd for C₄₅H₅₇N₅O₈ [M+H]⁺ m/z=796.4, found 796.

### Step 9: Preparation of (4-((4-amino-7-(4-(2-aminoethyl) benzyl)-2-(ethoxymethyl)-1H-imidazo[4,5-c]quinolin-1-yl) methyl) phenyl) methanol (Compound 5)

To a solution of di-tert-butyl (7-(4-(2-aminoethyl)benzyl)-1-(4-(((tert-butoxycarbonyl)oxy)methyl)benzyl)-2-(ethoxymethyl)-1H-imidazo[4,5-c]quinolin-4-yl)iminodicarbonate (100 mg, 0.126 mmol) in DCM (2 mL) was added TFA (1 mL) dropwise at 0′C. The mixture was stirred at RT for 2 hours. LCMS showed the reaction was completed. The reaction was concentrated and purified by Prep-HPLC (Column: Gemini-C18, 150×21.2 mm, 5 μm; Mobile Phase: ACN-H₂O (0.1% FA), Gradient: 10-40) to give Compound 5 (12.02 mg, 19.31% yield) as white solid.

LCMS (ESI) calcd for C₃₀H₃₃N₅O₂ [M+H]⁺ m/z=496.3, found 496.

¹H NMR (400 MHz, DMSO-d₆) δ 8.21 (s, 1H), 7.67 (d, J=8.4 Hz, 1H), 7.38 (s, 1H), 7.22 (dd, J=8.1, 2.8 Hz, 4H), 7.15 (d, J=8.0 Hz, 2H), 6.99 (d, J=8.1 Hz, 2H), 6.91 (dd, J=8.4, 1.5 Hz, 1H), 6.65 (s, 2H), 5.85 (s, 2H), 4.75 (s, 2H), 4.41 (s, 2H), 3.94 (s, 2H), 3.51-3.44 (m, 4H), 3.04-2.91 (m, 2H), 2.84-2.73 (m, 2H), 1.23 (s, 1H), 0.97 (t, J=7.0 Hz, 3H).

### Example 25

### Synthesis of Compound 46

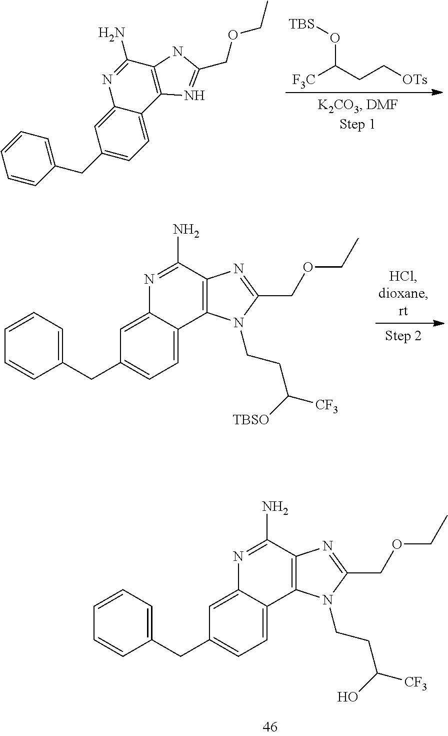

### Step 1: Preparation of 7-benzyl-1-(3-((tert-butyldimethylsilyl)oxy)-4,4,4-trifluorobutyl)-2-(ethoxymethyl)-1H-imidazo[4,5-c]quinolin-4-amine

A mixture of 7-benzyl-2-(ethoxymethyl)-1H-imidazo[4,5-c]quinolin-4-amine (120 mg, 0.37 mmol), 3-(bromomethyl)benzonitrile (220 mg, 0.53 mmol) and K₂CO₃ (160 mg, 1.1 mmol) in DMF (6 mL) was stirred at room temperature for 3 hours. LCMS showed the reaction was completed. The resulting mixture was diluted with water (20 mL) and extracted with EA (30 mL×3). The organic phases were washed with brine, dried over Na₂SO₄, concentrated and purified by column chromatography on silica gel (PE/EtOAc=1/4) to give 7-benzyl-1-(3-((tert-butyldimethylsilyl)oxy)-4,4,4-trifluorobutyl)-2-(ethoxymethyl)-1H-imidazo[4,5-c]quinolin-4-amine (40 mg, 18.9%) as a yellow solid.

LCMS (ESI) calcd for C₃₀H₃₉F₃N₄O₂Si [M+H]⁺ m/z=573.5, found 573.

### Step 2: Preparation of 4-(4-amino-7-benzyl-2-(ethoxymethyl)-1H-imidazo[4,5-c]quinolin-1-yl)-1,1,1-trifluorobutan-2-ol (Compound 46)

A solution of 7-benzyl-1-(3-((tert-butyldimethylsilyl)oxy)-4,4,4-trifluorobutyl)-2-(ethoxymethyl)-1H-imidazo[4,5-c]quinolin-4-amine (40 mg, 0.07 mmol) in HCl (5 mL, in dioxane) was stirred at RT for 4 hours. It was concentrated to give the crude. The residue was purified by Prep-HPLC (column: Gemini-C18 150×21.2 mm, 5 um; mobile phase: ACN-H₂O (0.1% FA), gradient: 10-50) to give the title compound (0.5 mg).

LCMS (ESI) calcd for C₂₄H₂₅F₃N₄O₂ [M+H]⁺ m/z=459.4, found 459.

¹H NMR (400 MHz, DMSO-d₆) δ 8.05 (d, J=8.2 Hz, 1H), 7.42 (s, 1H), 7.35-7.24 (m, 4H), 7.23-7.04 (m, 2H), 6.62 (d, J=5.6 Hz, 1H), 6.44 (s, 2H), 4.78 (s, 2H), 4.64 (m, 2H), 4.10 (s, 3H), 3.55 (m, 2H), 2.18 (s, 1H), 1.99 (s, 1H), 1.15 (t, J=7.0 Hz, 3H).

### Example 26

### Synthesis of Compound 48

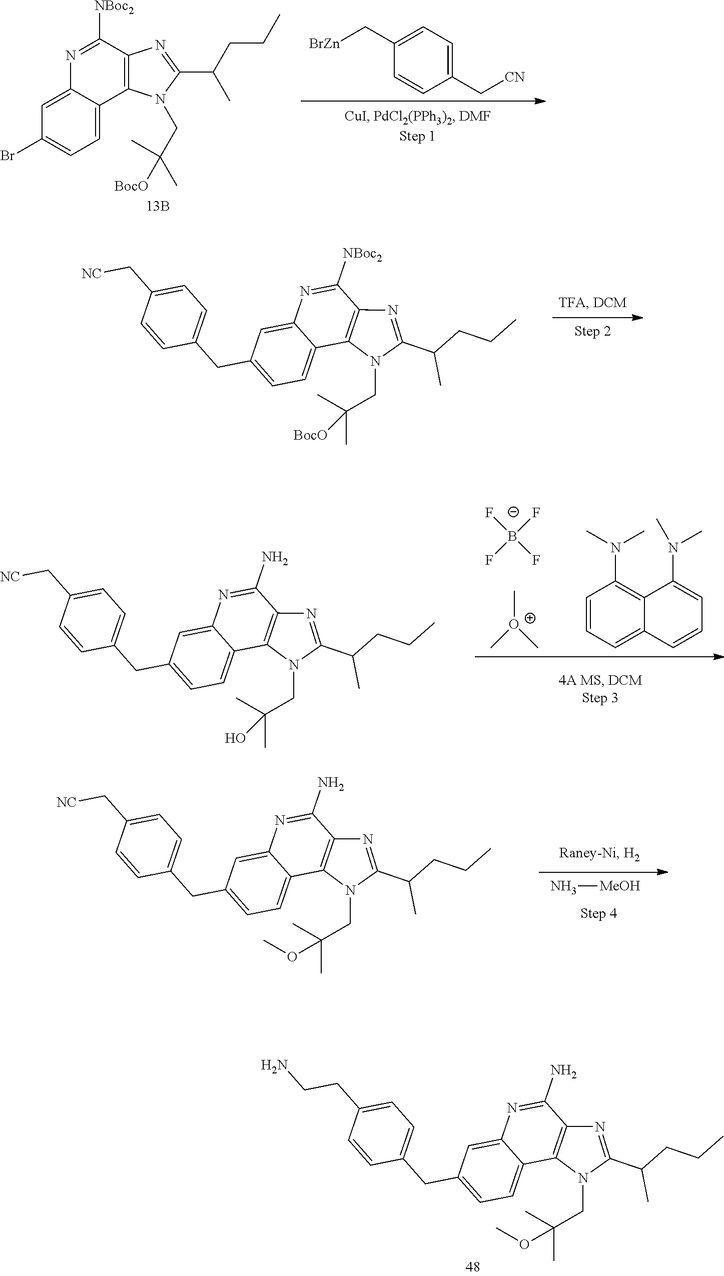

### Step 1: di-tert-butyl (1-(2-((tert-butoxycarbonyl)oxy)-2-methylpropyl)-7-(4-(cyanomethyl)benzyl)-2-(pentan-2-yl)-1H-imidazo[4,5-c]quinolin-4-yl)iminodicarbonate

A mixture of Zn powder (1.21 g, 18.60 mmol) and I₂ (250 mg, 0.98 mmol) in a 100 mL three-necked flask was evacuated and backfilled with N₂ three times and then charged with N₂. The flask was heated carefully with a hot air blower until I₂ was sublimated. Then a solution of 2-(4-(bromomethyl)phenyl)acetonitrile (1.0 g, 4.7 mmol) in DMF (6 mL) was added intermediately via a syringe. The mixture was stirred for 10 minutes while keeping the temperature was about 70° C. Then the heater and stirrer were removed. The upper layer clear solution was added dropwise into a stirred solution of di-tert-butyl (7-bromo-1-(2-((tert-butoxycarbonyl)oxy)-2-methylpropyl)-2-(pentan-2-yl)-1H-imidazo[4,5-c]quinolin-4-yl)iminodicarbonate (500 mg, 0.71 mmol), CuI (30 mg, 0.15 mmol) and Pd(pph₃)₂Cl₂ (100 mg, 0.15 mmol) in DMF (5 mL) under an atmosphere of N₂. The mixture was heated at 50° C. for 1 hour. Then the mixture was quenched with water (50 mL) and extracted with EtOAc (30 mL×3). The combined organic layers were washed with water and brine, dried over Na₂SO₄, filtered, concentrated to give the crude. The residue was purified by silica gel column chromatography (PE:EA=10:1) to afford desired product (300 mg, 55.8% yield) as a white solid.

LCMS (ESI) calcd for C₄₃H₅₇N₅O₇ [M+H]⁺ m/z=756.4, found 756.

### Step 2: 2-(4-((4-amino-1-(2-hydroxy-2-methylpropyl)-2-(pentan-2-yl)-1H-imidazo[4,5-c]quinolin-7-yl)methyl)phenyl)acetonitrile

To a solution of di-tert-butyl (1-(2-((tert-butoxycarbonyl)oxy)-2-methylpropyl)-7-(4-(cyanomethyl)benzyl)-2-(pentan-2-yl)-1H-imidazo[4,5-c]quinolin-4-yl)iminodicarbonate (300 mg, 0.3969 mmol) in DCM (6 mL) was added a solution of TFA (2 mL) dropwise. The reaction mixture was stirred at RT for 3 hrs. LCMS showed the reaction was completed. The resulting mixture was adjusted to pH=8 with NaHCO₃ and extracted with EtOAc (30 mL×3). The combined organic layers were washed with water and brine, dried over Na₂SO₄, filtered, concentrated to give the crude. The residue was purified by silica gel column chromatography (PE:EA 1:1) to give the title compound (180 mg, 89.5% yield) as a white solid.

LCMS (ESI) calcd for C₂₈H₃₃N₅O [M+H]⁺ m/z=456.2, found 456.

### Step 3: 2-(4-((4-amino-J-(2-methoxy-2-methylpropyl)-2-(pentan-2-yl)-1H-imidazo[4,5-c]quinolin-7-yl)methyl)phenyl)acetonitrile

To a solution of 2-(4-((4-amino-1-(2-hydroxy-2-methylpropyl)-2-(pentan-2-yl)-1H-imidazo[4,5-c]quinolin-7-yl)methyl)phenyl)acetonitrile (180 mg, 0.40 mmol), N₁,N₁,N₈,N₈-tetramethylnaphthalene-1,8-diamine (508 mg, 2.37 mmol) and 4A Molecular Sieves (200 mg) in DCM stirred at 25° C. was added a solution of trimethyloxonium tetrafluoroborate (100 mg, 0.6716 mmol) dropwise. The reaction mixture was stirred at RT for 16 hrs. LCMS showed the reaction was completed. The reaction was diluted with water and extracted with EtOAc (40 mL×3). The combined organic layers were washed with water and brine, dried over Na₂SO₄, filtered, concentrated to give the crude. The residue was purified by silica gel column chromatography (PE:EA 1:1) to give the title compound (30 mg, 14.55% yield) as a white solid.

LCMS (ESI) calcd for C₂₉H₃₅N₅O [M+H]⁺ m/z=470.2, found 470.

### Step 4: 7-(4-(2-aminoethyl)benzyl)-1-(2-methoxy-2-methylpropyl)-2-(pentan-2-yl)-1H-imidazo[4,5-c]quinolin-4-amine (Compound 48)

To a solution of 2-(4-((4-amino-1-(2-methoxy-2-methylpropyl)-2-(pentan-2-yl)-1H-imidazo[4,5-c]quinolin-7-yl)methyl)phenyl)acetonitrile (30 mg, 0.064 mmol) in NH₃-MeOH (10 mL, 7M) was added a solution of Raney-Ni in MeOH dropwise. The reaction mixture was stirred at RT for 1 h under H₂. The mixture was filtered through celite and the filtrate was concentrated under vacuum. The crude was purified by Prep-HPLC (column: Gemini-C18 150×21.2 mm, 5 um; mobile phase: ACN-H₂O (0.1% FA); gradient: 10-50) to afford Compound 48 (2.17 mg, 7.04% yield) as white solid.

LCMS (ESI) calcd for C₂₉H₃₉N₅O [M+H]⁺ m/z=474.3, found 474.

1H NMR (400 MHz, DMSO-d₆) δ 8.34 (d, J=8.5 Hz, 1H), 7.77 (s, 1H), 7.32-7.20 (m, 4H), 7.16 (d, J=8.0 Hz, 2H), 4.09 (s, 2H), 3.87 (s, 3H), 3.46 (m, 2H), 2.94-2.82 (m, 2H), 2.75 (dd, J=13.8, 5.4 Hz, 3H), 1.21 (m, 16H), 0.85 (s, 3H).

### Example 27

### Synthesis of Compounds 49 and 50

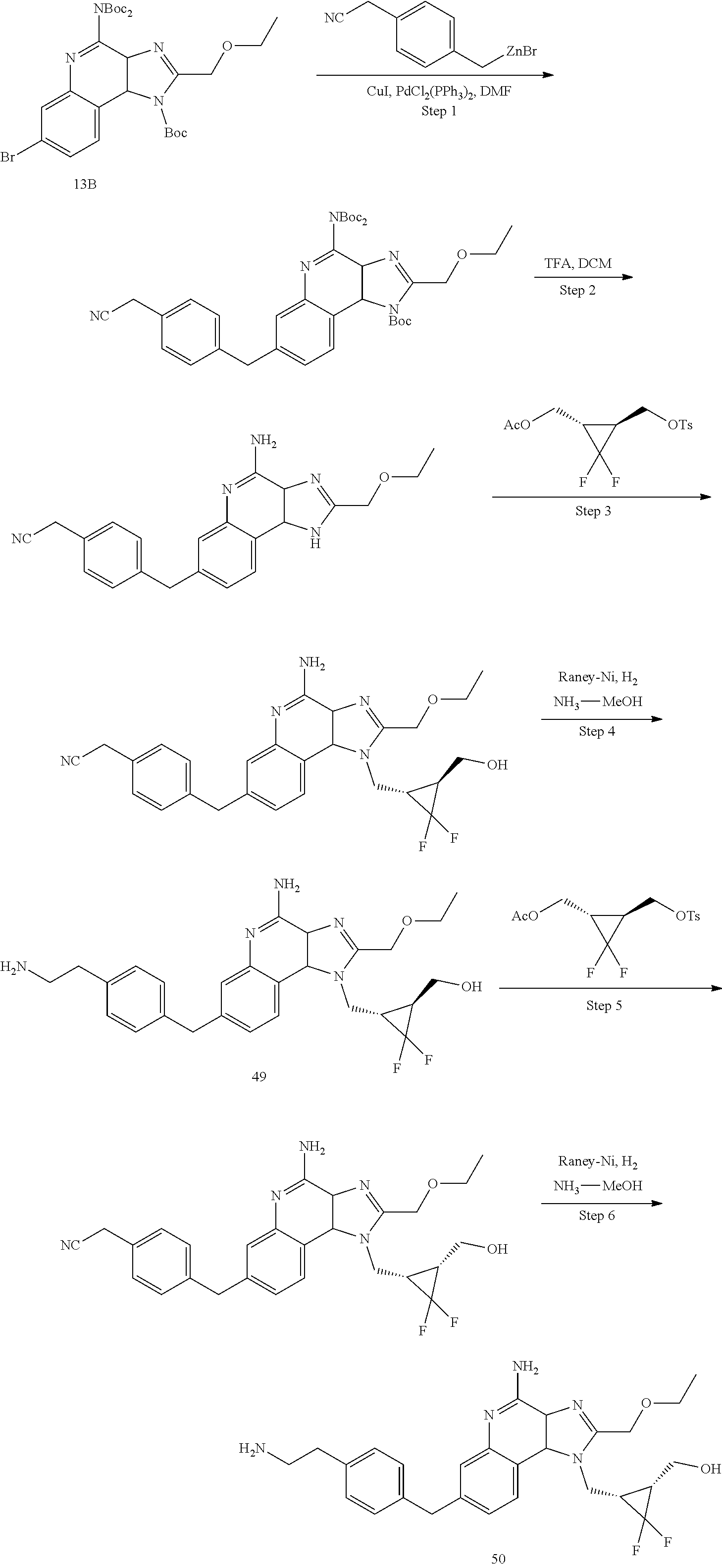

### Step 1: [1-(4-{bis[(tert-butoxy)carbonyl]amino]-7-{[4-(cyanomethyl)phenyl]methyl}-2-[(2R)-pentan-2-yl]imidazo[4,5-c]quinolin-1-yl)-2-methylpropan-2-yl]tert-butyl carbonate

A solution of (4-(cyanomethyl)benzyl)zinc(II) bromide was added into a stirred solution of tert-butyl 4-(bis(tert-butoxycarbonyl)amino)-7-bromo-2-(ethoxymethyl)-1H-imidazo[4,5-c]quinoline-1-carboxylate (500 mg, 0.71 mmol), CuI (60 mg, 0.28 mmol) and Pd(pph₃)₂Cl₂ (100 mg, 0.14 mmol) in DMF (5 mL) under an atmosphere of N₂. The mixture was heated at 50° C. for 1 hour. Then the mixture was quenched with water (50 mL) and extracted with EtOAc (30 mL×3). The combined organic layers were washed with water and brine, dried over Na₂SO₄, filtered, concentrated to give the crude. The residue was purified by silica gel column chromatography (PE:EA 5:1) to afford desired product (300 mg, 55.8% yield) as a yellow solid.

LCMS (ESI) calcd for C₄₃H₅₇N₅O₇ [M+H]⁺ m/z=756.4, found 756.

### Step 2: 2-(4-((4-amino-2-(ethoxymethyl)-1H-imidazo[4,5-c]quinolin-7-yl)methyl)phenyl)acetonitrile

To a solution of tert-butyl 4-(bis(tert-butoxycarbonyl)amino)-7-(4-(cyanomethyl)benzyl)-2-(ethoxymethyl)-1H-imidazo[4,5-c]quinoline-1-carboxylate (300 mg, 0.40 mmol) in DCM (6 mL) was added TFA (2 mL) dropwise at 0° C. The reaction mixture was stirred at RT for 3 hours. LCMS showed the reaction was completed. The resulting mixture was adjusted to pH=8 with NaHCO₃ and extracted with EtOAc (30 mL×3). The combined organic layers were washed with water and brine, dried over Na₂SO₄, filtered, concentrated to give the crude. The residue was purified by silica gel column chromatography (PE:EA 1:1) to afford desired product (180 mg, 89.5% yield) as a white solid.

LCMS (ESI) calcd for C₂₈H₃₃N₅O [M+H]⁺ m/z=456.2, found 456.

### Step 3: 2-(4-((4-amino-1-(((1S,3R)-2,2-difluoro-3-(hydroxymethyl)cyclopropyl)methyl)-2-(ethoxymethyl)-1H-imidazo[4,5-c]quinolin-7-yl)methyl)phenyl)acetonitrile

To a solution of 2-(4-((4-amino-2-(ethoxymethyl)-1H-imidazo[4,5-c]quinolin-7-yl)methyl)phenyl)acetonitrile (100 mg, 0.27 mmol) and K₂CO₃ (112 mg, 0.81 mmol) in DMF (4 mL) was ((1S,3R)-2,2-difluoro-3-((tosyloxy)methyl)cyclopropyl)methyl acetate (180 mg, 0.54 mmol). The reaction mixture was stirred at RT for 4 hours. LCMS showed the reaction was completed. The reaction was diluted with water and extracted with EtOAc (50 mL*3). The combined organic layers were washed with water and brine, dried over Na₂SO₄, filtered, concentrated to give the crude. The residue was purified by silica gel column chromatography (PE:EA 1:1) to give the title compound (15 mg, 10.2% yield) as a white solid.

LCMS (ESI) calcd for C₂₇H₂₇F₂N₅O₂[M+H]⁺ m/z=492.2, found 492.

### Step 4: ((1S,3S)-3-((4-amino-7-(4-(2-aminoethyl)benzyl)-2-(ethoxymethyl)-1H-imidazo[4,5-c]quinolin-1-yl)methyl)-2,2-difluorocyclopropyl)methanol (Compound 49)

To a solution of 2-(4-((4-amino-1-(((1S,3S)-2,2-difluoro-3-(hydroxymethyl)cyclopropyl)methyl)-2-(ethoxymethyl)-1H-imidazo[4,5-c]quinolin-7-yl)methyl)phenyl)acetonitrile (15 mg, 0.03 mmol) in NH₃-MeOH (4 mL, 7M) was added a solution of Raney-Ni in MeOH dropwise. The reaction mixture was stirred at RT for 1 h. The mixture was filtered through celite and the filtrate was concentrated under vacuum. The crude was purified by Prep-HPLC (column: Gemini-C18 150×21.2 mm, 5 um; mobile phase: ACN-H₂O (0.1% FA); gradient: 10-50) to afford Compound 49 (0.12 mg, 0.66% yield) as white solid.

LCMS (ESI) calcd for C₂₇H₃₁F₂N₅O₂ [M+H]⁺ m/z=496.2, found 496.

Step 5 and step 6: procedures for step 5 and step 6 were similar with that of step 3 and 4.

### ((1R,3S)-3-((4-amino-7-(4-(2-aminoethyl)benzyl)-2-(pentan-2-yl)-1H-imidazo[4,5-c]quinolin-1-yl)methyl)-2,2-difluorocyclopropyl)methanol (Compound 50)

LCMS (ESI) calcd for C₂₇H₃₁F₂N₅O₂[M+H]⁺ m/z=508.3, found 508.

### Example 28

### Synthesis of Compound 56

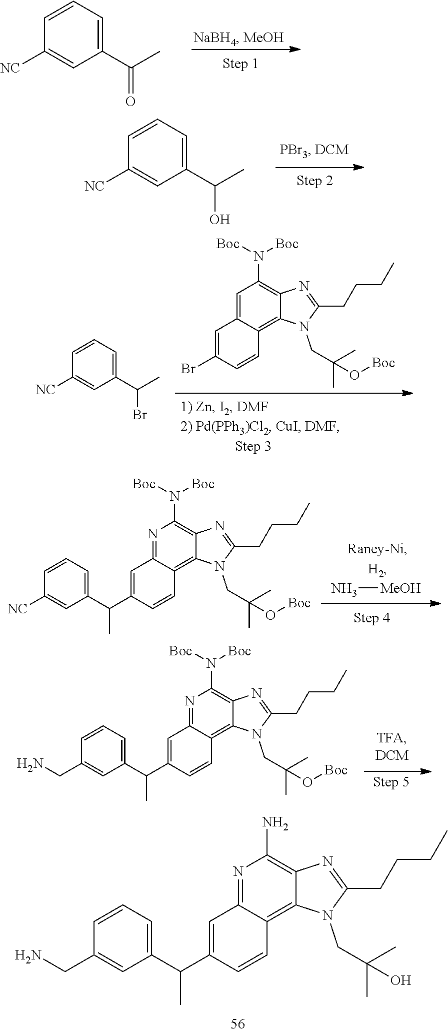

### Step 1. Preparation of 3-(1-hydroxyethyl)benzonitrile

To a solution of 3-acetylbenzonitrile (10.0 g, 68.9 mmol) in MeOH (230 mL) at 0° C. under N₂ was added NaBH₄ (2.61 g, 68.9 mmol) in portions. After 2 hours of gradual warming to room temperature, 1N HCl was added to the solution dropwise. Then the organic solvent was removed under reduced pressure. DCM (50 mL) was added, and the layers were separated. The aqueous phase was extracted with DCM (50 mL*3). The combined extracts were washed with NaHCO₃ (50 mL), saturated NaCl, dried over Na₂SO₄, concentrated under vacuum to give 3-(1-hydroxyethyl)benzonitrile (8.0 g, 70.9%) as a yellow oil.

LCMS (ESI) calcd for C₉H₁₀NO [M+H]⁺ m/z=148.08, found 148.00.

### Step 2. Preparation of 3-(1-bromoethyl)benzonitrile

To a solution of 3-(1-hydroxyethyl)benzonitrile (7 g, 47.6 mmol) in DCM (200 mL) was added PBr₃(5.4 mL) at 0° C. The mixture was stirred at RT for 1 hour. Then the solution was quenched with water and extracted with DCM, washed with NaHCO₃ (aq) and saturated NaCl, dried over Na₂SO₄, concentrated under vacuum to give the crude. The residue was purified by silica gel column chromatography (PE:EtOAc 10:1) to give 3-(1-bromoethyl)benzonitrile (5.0 g, 44.9% yield) as a yellow oil.

### Step 3. Preparation of tert-butyl (tert-butoxycarbonyl)(1-(2-((tert-butoxycarbonyl)oxy)-2-methylpropyl)-2-butyl-7-(1-(3-cyanophenyl)ethyl)-1H-imidazo[4,5-c]quinolin-4-yl)carbamate

A mixture of Zn power (2 g, 31 mmol) and I₂ (300 mg, 1.2 mmol) in a 100 mL three-necked flask was evacuated and backfilled with N₂ three times and then charged with N₂. The flask was heated carefully with a hot air blower until 12 was sublimated. Then a solution of 3-(1-bromoethyl)benzonitrile (0.76 g, 3.6 mmol) in DMF (6 mL) was added intermediately via a syringe. The mixture was stirred for 10 minutes while keeping the temperature was about 70° C. Then the heater and stirrer were removed. The upper layer clear solution was added dropwise into a stirred solution of tert-butyl (7-bromo-1-(2-((tert-butoxycarbonyl)oxy)-2-methylpropyl)-2-butyl-1H-imidazo[4,5-c]quinolin-4-yl)(tert-butoxycarbonyl)carbamate (250 mg, 0.36 mmol), CuI (66 mg, 0.36 mmol) and Pd(pph₃)₂Cl₂ (60 mg, 0.1 mmol) in DMF (3 mL) under an atmosphere of N₂. The mixture was heated at 80 TC for 1 hour. Then the mixture was quenched with water (100 mL) and extracted with EtOAc (50 mL×3). The combined organic layers were washed with water and brine, dried over Na₂SO₄, filtered, concentrated to give the crude. The residue was purified by silica gel column chromatography (PE:EtOAc 2:1) to afford tert-butyl (tert-butoxycarbonyl)(1-(2-((tert-butoxycarbonyl)oxy)-2-methylpropyl)-2-butyl-7-(1-(3-cyanophenyl)ethyl)-1H-imidazo[4,5-c]quinolin-4-yl)carbamate (210 mg, 60% yield) as yellow solid.

LCMS (ESI) calcd for C₃₇H₄₈N₅O₅[M+H-Boc]⁺ m/z=642.37, found 642.25.

### Step 4. Preparation of tert-butyl (7-(1-(3-(aminomethyl)phenyl)ethyl)-1-(2-((tert-butoxycarbonyl)oxy)-2-methylpropyl)-2-butyl-1H-imidazo[4,5-c]quinolin-4-yl)(tert-butoxycarbonyl)carbamate

To a solution of tert-butyl (tert-butoxycarbonyl)(1-(2-((tert-butoxycarbonyl)oxy)-2-methylpropyl)-2-butyl-7-(1-(3-cyanophenyl)ethyl)-1H-imidazo[4,5-c]quinolin-4-yl)carbamate (210 mg, 0.28 mmol) in NH₃-MeOH (5 mL) was added the right amount of Raney-Ni in MeOH. The mixture was stirred at RT for 4 hours under H₂. LCMS showed the reaction was completed. Then the mixture was filtered through celite and concentrated under vacuo to give the crude tert-butyl (7-(1-(3-(aminomethyl)phenyl)ethyl)-1-(2-((tert-butoxycarbonyl)oxy)-2-methylpropyl)-2-butyl-1H-imidazo[4,5-c]quinolin-4-yl)(tert-butoxycarbonyl)carbamate (180 mg, 76.7% yield) which was used in the next step without further purification.

LCMS (ESI) calcd. for C₃₇H₅₁N₅O₅[M+H−Boc]⁺ m/z 646.40, found 646.35.

### Step 5. Preparation of 1-(4-amino-7-(1-(3-(aminomethyl)phenyl)ethyl)-2-butyl-1H-imidazo[4,5-c]quinolin-1-yl)-2-methylpropan-2-ol (Compound 56)

To a solution of tert-butyl (7-(1-(3-(aminomethyl)phenyl)ethyl)-1-(2-((tert-butoxycarbonyl)oxy)-2-methylpropyl)-2-butyl-1H-imidazo[4,5-c]quinolin-4-yl)(tert-butoxycarbonyl)carbamate (180 mg, 0.24 mmol) in DCM (5 mL) was added TFA (1 mL) at room temperature. The reaction mixture was stirred at room temperature for 2 hours. After the reaction was completed, the mixture was concentrated. The residue was dissolved in water (40 mL) then adjusted to pH=8-9 with saturated aqueous NaHCO₃. The resulting solution was extracted with DCM (30 mL×3). The combined organic phases were washed with brine, dried over sodium sulfate, concentrated under vacuum. The residue was purified by prep-HPLC (Gemini 5 um C18 column, 150*21.2 mm, eluting with 15% to 40% MeCN/H₂O containing 0.1% TFA) to provide 1-(4-amino-7-(1-(3-(aminomethyl)phenyl)ethyl)-2-butyl-1H-imidazo[4,5-c]quinolin-1-yl)-2-methylpropan-2-ol (Compound 56) (25 mg, 22.2% yield) as a white solid.

LCMS (ESI) calcd for C₂₇H₃₆N₅O [M+H]⁺ m/z=446.29, found 446.10.

¹H NMR (400 MHz, DMSO-d₆, ppm) δ 8.13 (d, J=8.6 Hz, 1H), 7.43 (s, 1H), 7.29-7.05 (m, 5H), 6.31 (s, 2H), 4.75 (s, 1H), 4.47 (s, 2H), 4.21 (q, J=7.1 Hz, 1H), 3.68 (s, 2H), 3.05-2.85 (m, 2H), 1.83-1.69 (m, 2H), 1.64 (d, J=7.2 Hz, 3H), 1.46-1.36 (m, 2H), 1.14 (s, 6H), 0.93 (t, J=7.3 Hz, 3H).

### Example 29

### Synthesis of Compound 57

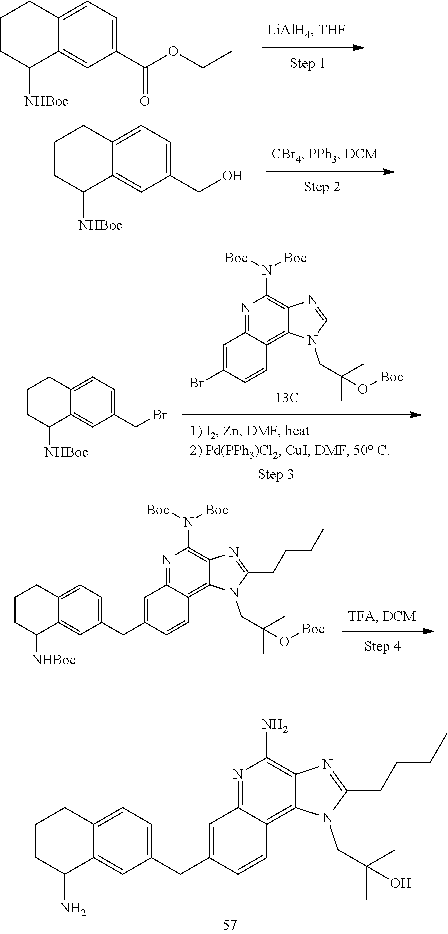

### Step 1: Preparation of tert-butyl (7-(hydroxymethyl)-1,2,3,4-tetrahydronaphthalen-1-yl)carbamate

To a solution of ethyl 8-((tert-butoxycarbonyl)amino)-5,6,7,8-tetrahydronaphthalene-2-carboxylate (800 mg, 2.51 mmol) in THF (10 mL) was added LiAlH₄ (1 M in THF, 5 mL) dropwise. The mixture was stirred at room temperature for 1 hours. LCMS showed the reaction was completed. The resulting solution was quenched with water and extracted with EtOAc (60 mL×3). The combined organic phases were washed with brine, dried over Na₂SO₄, concentrated under vacuum. The residue was purified by flash column chromatography on silica gel (PE/EtOAc=1/1) to give tert-butyl (7-(hydroxymethyl)-1,2,3,4-tetrahydronaphthalen-1-yl)carbamate (700 mg, 95.68% yield).

LCMS (ESI) calcd. for C₁₆H₂₃NO₃ [M+H]⁺ m/z 278.18, found [M+H+Na]⁺ m/z 299.95.

### Step 2: Preparation of tert-butyl (7-(bromomethyl)-1,2,3,4-tetrahydronaphthalen-1-yl)carbamate

To a solution of tert-butyl (7-(hydroxymethyl)-1,2,3,4-tetrahydronaphthalen-1-yl)carbamate (700 mg, 2.52 mmol) and PPh₃ (792 mg, 3.02 mmol) in DCM (8 mL) was added CBr₄ (1 g, 3.02 mmol) at 0° C. The mixture was stirred at room temperature for 1 hours. LCMS showed the reaction was completed. The resulting solution was concentrated under vacuum. The residue was purified by flash column chromatography on silica gel (PE/EtOAc=5/1) to give tert-butyl (7-(bromomethyl)-1,2,3,4-tetrahydronaphthalen-1-yl)carbamate (750 mg, 88.24% yield).

LCMS (ESI) calcd. for C₁₆H₂₂BrNO₂ [M+H−tBu]⁺ m/z 284.09, found m/z 283.90.

### Step 3: Preparation of tert-butyl (tert-butoxycarbonyl)(7-((8-((tert-butoxycarbonyl)amino)-5,6,7,8-tetrahydronaphthalen-2-yl)methyl)-1-(2-((tert-butoxycarbonyl)oxy)-2-methylpropyl)-2-butyl-1H-imidazo[4,5-c]quinolin-4-yl)carbamate

A mixture of Zn power (620 mg, 9.59 mmol) and I₂ (200 mg, 0.79 mmol) in a 250 mL three-necked flask was evacuated and backfilled with N₂ three times and then charged with N₂. The flask was heated carefully with a hot air blower until I₂ was sublimated. Then a solution of tert-butyl (7-(bromomethyl)-1,2,3,4-tetrahydronaphthalen-1-yl)carbamate (500 mg, 1.47 mmol) in DMF (5 mL) was added intermediately via a syringe. The mixture was stirred for 10 minutes while keeping the temperature was about 80° C. Then the heater and stirrer were removed. The upper layer clear solution was added dropwise into a stirred solution of tert-butyl (7-bromo-1-(2-((tert-butoxycarbonyl)oxy)-2-methylpropyl)-2-butyl-1H-imidazo[4,5-c]quinolin-4-yl)(tert-butoxycarbonyl)carbamate (200 mg, 0.29 mmol), CuI (11 mg, 0.06 mmol) and Pd(pph₃)₂Cl₂ (21 mg, 0.03 mmol) in DMF (3 mL) under an atmosphere of N₂. The mixture was heated at 50° C. for 1 hour. Then the mixture was quenched with water (50 mL) and extracted with EtOAc (50 mL×3). The combined organic phases were washed with brine, dried over Na₂SO₄, concentrated under vacuum. The residue was purified by flash column chromatography on silica gel (PE/EtOAc=2/1) to give tert-butyl (tert-butoxycarbonyl)(7-((8-((tert-butoxycarbonyl)amino)-5,6,7,8-tetrahydronaphthalen-2-yl)methyl)-1-(2-((tert-butoxycarbonyl)oxy)-2-methylpropyl)-2-butyl-1H-imidazo[4,5-c]quinolin-4-yl)carbamate (150 mg, 59.52% yield) as a yellow solid.

LCMS (ESI) calcd. for C₄₉H₆₉N₅O₉ [M+H]⁺ m/z 872.52, found 872.95.

### Step 4: Preparation of tert-butyl (tert-butoxycarbonyl)(7-((8-((tert-butoxycarbonyl)amino)-5,6,7,8-tetrahydronaphthalen-2-yl)methyl)-1-(2-((tert-butoxycarbonyl)oxy)-2-methylpropyl)-2-butyl-1H-imidazo[4,5-c]quinolin-4-yl)carbamate (Compound 57)

To a solution of tert-butyl (tert-butoxycarbonyl)(7-((8-((tert-butoxycarbonyl)amino)-5,6,7,8-tetrahydronaphthalen-2-yl)methyl)-1-(2-((tert-butoxycarbonyl)oxy)-2-methylpropyl)-2-butyl-1H-imidazo [4,5-c]quinolin-4-yl)carbamate 7 (150 mg, 0.17 mmol) in DCM (1.5 mL) was added TFA (0.5 mL) at room temperature. The reaction mixture was stirred at room temperature for 2 hours. After the reaction was completed, the mixture was concentrated. The residue was diluted with water t and adjusted to pH=8-9 with saturated aqueous NaHCO₃. The resulting solution was extracted with DCM (10 mL×3). The combined organic phases were washed with brine, dried over Na₂SO₄, concentrated under vacuum. The residue was purified by prep-HPLC (Gemini 5 um C₁₈ column, 150*21.2 mm, eluting with 40% to 85% MeCN/H₂O containing 0.1% TFA) to provide 1-(4-amino-7-((8-amino-5,6,7,8-tetrahydronaphthalen-2-yl)methyl)-2-butyl-1H-imidazo[4,5-c]quinolin-1-yl)-2-methylpropan-2-ol (Compound 57) (30 mg, 37.04% yield) as a white solid.

LCMS (ESI) calcd. for C₂₉H₃₇N₅O [M+H]⁺ m/z 472.31, found 472.20.

¹H NMR (400 MHz, DMSO-d₆, ppm) δ 14.04 (s, 1H), 9.01 (s, 2H), 8.41 (d, J=8.6 Hz, 1H), 8.29 (s, 3H), 7.57 (s, 1H), 7.43 (s, 1H), 7.33 (d, J=8.6 Hz, 1H), 7.18 (dd, J=24.0, 7.9 Hz, 2H), 4.41 (d, J=4.6 Hz, 2H), 4.08 (d, J=3.1 Hz, 3H), 3.04 (t, J=7.7 Hz, 2H), 2.79-2.66 (m, 2H), 2.07-1.99 (m, 1H), 1.94-1.68 (m, 5H), 1.50-1.37 (m, 2H), 1.19 (s, 6H), 0.94 (t, J=7.3 Hz, 3H).

### Example 30

### Synthesis of Compound 58

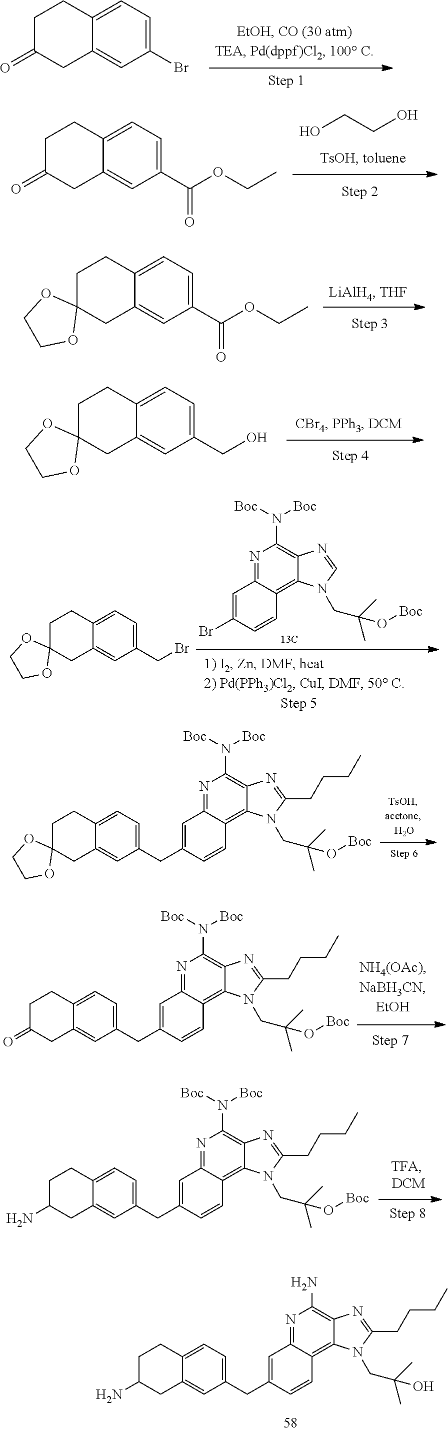

### Step 1: Preparation of ethyl 7-oxo-5,6,7,8-tetrahydronaphthalene-2-carboxylate

A solution of 7-bromo-3,4-dihydronaphthalen-2(1H)-one (4 g, 17.8 mmol), TEA (9 g, 89 mmol) and Pd(dppf)Cl₂ (0.39 g, 0.54 mmol) in EtOH (30 mL) was heated at 100° C. under an atmosphere of CO (30 atm) for 16 hours in a high-pressure reactor. LCMS showed the reaction was completed. The resulting solution was diluted with water (90 mL) and extracted with EtOAc (50 mL×3). The combined organic phases were washed with brine, dried over Na₂SO₄ and concentrated under vacuum. The residue was purified by flash column chromatography on silica gel (PE/EtOAc=3/1) to give ethyl 7-oxo-5,6,7,8-tetrahydronaphthalene-2-carboxylate (2.4 g, 64% yield).

LCMS (ESI) calcd. for C₁₃H₁₄O₃ [M+H]⁺ m/z 219.10, found 219.05.

### Step 2: Preparation of ethyl 3,4-dihydro-1H-spiro[naphthalene-2,2′-[1,3]dioxolane]-7-carboxylate

A solution of ethyl 7-oxo-5,6,7,8-tetrahydronaphthalene-2-carboxylate (2.4 g, 11 mmol), TsOH (100 mg) and ethane-1,2-diol (2.0 g, 33 mmol) in toluene (30 mL) was heated at 100° C. for 5 hours. The solution was concentrated and diluted with water, extracted with EtOAc (50 mL×3). The combined organic phases were washed with brine, dried over Na₂SO₄ and concentrated under vacuum. The residue was purified by flash column chromatography on silica gel (PE/EtOAc=5/1) to give ethyl 3,4-dihydro-1H-spiro[naphthalene-2,2′-[1,3]dioxolane]-7-carboxylate (2.7 g, 93.7%) as oil.

LCMS (ESI) calcd. for C₁₅H₁₉O₄[M+H]⁺ m/z 263.13.

### Step 3: Preparation of (3,4-dihydro-1H-spiro[naphthalene-2,2′-[1,3]dioxolan]-7-yl)methanol

To a solution of ethyl 3,4-dihydro-1H-spiro[naphthalene-2,2′-[1,3]dioxolane]-7-carboxylate (2.5 g, 9.5 mmol) in THF (50 mL) was added LiAlH₄ (1 M in THF, 10 mL) dropwise at 0′C. The mixture was stirred at room temperature for 1 hour. LCMS showed the reaction was completed. The resulting solution was quenched with water and extracted with EtOAc (60 mL×3). The combined organic phases were washed with brine, dried over Na₂SO₄, concentrated under vacuum. The residue was purified by flash column chromatography on silica gel (PE/EtOAc=3/1) to give (3,4-dihydro-1H-spiro[naphthalene-2,2′-[1,3]dioxolan]-7-yl)methanol (1.6 g, 76.9% yield).

LCMS (ESI) calcd. for C₁₃H₁₇O₃ [M+H]⁺ m/z 221.12.

### Step 4: Preparation of 7-(bromomethyl)-3,4-dihydro-1H-spiro[naphthalene-2,2′-[1,3]dioxolane]

To a solution of (3,4-dihydro-1H-spiro[naphthalene-2,2′-[1,3]dioxolan]-7-yl)methanol (1.6 g, 7.27 mmol) and PPh₃ (1.9 g, 7.27 mmol) in DCM (8 mL) was added CBr₄ (2.41 g, 7.27 mmol) at 0° C. The mixture was stirred at room temperature for 1 hour. LCMS showed the reaction was completed. The resulting solution was concentrated under vacuum. The residue was purified by flash column chromatography on silica gel (PE/EtOAc=5/1) to give 7-(bromomethyl)-3,4-dihydro-1H-spiro[naphthalene-2,2′-[1,3]dioxolane](1.64 g, 80% yield).

### Step 5: Preparation of tert-butyl (tert-butoxycarbonyl)(1-(2-((tert-butoxycarbonyl)oxy)-2-methylpropyl)-2-butyl-7-((3,4-dihydro-1H-spiro[naphthalene-2,2′-[1,3]dioxolan]-7-yl)methyl)-1H-imidazo[4,5-c]quinolin-4-yl)carbamate

A mixture of Zn power (1 g, 14.5 mmol) and I₂ (438 mg, 1.7 mmol) in a 100 mL three-necked flask was evacuated and backfilled with N₂ three times and then charged with N₂. The flask was heated carefully with a hot air blower until I₂ was sublimated. Then a solution of 7-(bromomethyl)-3,4-dihydro-1H-spiro[naphthalene-2,2′-[1,3]dioxolane](1.63 g, 5.7 mmol) in DMF (6 mL) was added intermediately via a syringe. The mixture was stirred for 10 minutes while keeping the temperature was about 70° C. Then the heater and stirrer were removed. The upper layer clear solution was added dropwise into a stirred solution tert-butyl (7-bromo-1-(2-((tert-butoxycarbonyl)oxy)-2-methylpropyl)-2-butyl-1H-imidazo[4,5-c]quinolin-4-yl)(tert-butoxycarbonyl)carbamate (400 mg, 0.58 mmol), CuI (54.8 mg, 0.29 mmol) and Pd(pph₃)₂Cl₂ (81 mg, 0.12 mmol) in DMF (6 mL) under an atmosphere of N₂. The mixture was heated at 50° C. for 1.5 hours. Then the mixture was quenched with water (80 mL) and extracted with EtOAc (50 mL×3). The combined organic layers were washed with water and brine, dried over Na₂SO₄, filtered, concentrated to give the crude. The residue was purified by silica gel column chromatography (PE:EA 2:1) to afford tert-butyl (tert-butoxycarbonyl)(1-(2-((tert-butoxycarbonyl)oxy)-2-methylpropyl)-2-butyl-7-((3,4-dihydro-1H-spiro[naphthalene-2,2′-[1,3]dioxolan]-7-yl)methyl)-1H-imidazo[4,5-c]quinolin-4-yl)carbamate (340 mg, 65% yield) as yellow solid.

LCMS (ESI) calcd for C₄₆H₆₃N₄O₉ [M+H]⁺ m/z=815.46, found 815.45.

### Step 6: Preparation of tert-butyl (tert-butoxycarbonyl)(1-(2-((tert-butoxycarbonyl)oxy)-2-methylpropyl)-2-butyl-7-((7-oxo-5,6,7,8-tetrahydronaphthalen-2-yl)methyl)-1H-imidazo[4,5-c]quinolin-4-yl)carbamate

To a solution of tert-butyl (tert-butoxycarbonyl)(1-(2-((tert-butoxycarbonyl)oxy)-2-methylpropyl)-2-butyl-7-((3,4-dihydro-1H-spiro[naphthalene-2,2′-[1,3]dioxolan]-7-yl)methyl)-1H-imidazo[4,5-c]quinolin-4-yl)carbamate (300 mg, 0.37 mmol) in acetone:H₂O (1:1.10 mL) was added TsOH·H₂O (209 mg, 1.1 mmol) at RT. The solution was stirred at RT for overnight. LCMS showed the reaction was completed. The mixture was concentrated and diluted with water, extracted with EA (50 mL×3). The combine organic phases were washed with brine, dried over Na₂SO₄, concentrated to give the crude. The residue was purified by silica gel column chromatography (PE:EtOAc=2:1) to give the tert-butyl (tert-butoxycarbonyl)(1-(2-((tert-butoxycarbonyl)oxy)-2-methylpropyl)-2-butyl-7-((7-oxo-5,6,7,8-tetrahydronaphthalen-2-yl)methyl)-1H-imidazo[4,5-c]quinolin-4-yl)carbamate (120 mg, 38.1% yield).

LCMS (ESI) calcd for C₄₄H₅₉N₄O₈[M+H]⁺ m/z=771.44, found 771.45.

### Step 7: Preparation of tert-butyl (7-((7-amino-5,6,7,8-tetrahydronaphthalen-2-yl)methyl)-1-(2-((tert-butoxycarbonyl)oxy)-2-methylpropyl)-2-butyl-1H-imidazo[4,5-c]quinolin-4-yl)(tert-butoxycarbonyl) carbamate

A solution of tert-butyl (tert-butoxycarbonyl)(1-(2-((tert-butoxycarbonyl)oxy)-2-methylpropyl)-2-butyl-7-((7-oxo-5,6,7,8-tetrahydronaphthalen-2-yl)methyl)-1H-imidazo[4,5-c]quinolin-4-yl)carbamate (100 mg, 0.13 mmol), ammonium acetate (99.6 mg, 1.3 mmol) and NaBH₃CN (17 mg, 0.26 mmol) in EtOH (8 mL) was stirred at rt for 16 hours. LCMS showed the reaction was completed. The resulting solution was concentrated under vacuum and purified by flash column chromatography on silica gel (PE/EtOAc=1:1) to give tert-butyl (7-((7-amino-5,6,7,8-tetrahydronaphthalen-2-yl)methyl)-1-(2-((tert-butoxycarbonyl)oxy)-2-methylpropyl)-2-butyl-1H-imidazo[4,5-c]quinolin-4-yl)(tert-butoxycarbonyl)carbamate (60 mg, 53.95% yield).

LCMS (ESI) calcd. for C₄₄H₆₂N₅O₇ [M+H]⁺ m/z 772.47, found 772.45.

### Step 8. Preparation of 1-(4-amino-7-((7-amino-5,6,7,8-tetrahydronaphthalen-2-yl)methyl)-2-butyl-1H-imidazo[4,5-c]quinolin-1-yl)-2-methylpropan-2-ol (Compound 58)

To a solution of tert-butyl (7-((7-amino-5,6,7,8-tetrahydronaphthalen-2-yl)methyl)-1-(2-((tert-butoxycarbonyl)oxy)-2-methylpropyl)-2-butyl-1H-imidazo[4,5-c]quinolin-4-yl)(tert-butoxycarbonyl) carbamate (60 mg, 0.08 mmol) in DCM (3 mL) was added TFA (1 mL) at room temperature. The reaction mixture was stirred at room temperature for 6 hours. After the reaction was completed, the mixture was concentrated. The residue was dissolved in water and then adjusted to pH=8-9 with saturated aqueous NaHCO₃. The resulting solution was extracted with DCM (10 mL×3). The combined organic phases were washed with brine, dried over Na₂SO₄, concentrated under vacuum. The residue was purified by prep-HPLC (Gemini 5 um C18 column, 150*21.2 mm, eluting with 40% to 85% MeCN/H₂O containing 0.1% TFA) to provide 1-(4-amino-7-((7-amino-5,6,7,8-tetrahydronaphthalen-2-yl)methyl)-2-butyl-1H-imidazo[4,5-c]quinolin-1-yl)-2-methylpropan-2-ol (Compound 58) (7 mg, 18.2% yield) as a white solid.

LCMS (ESI) calcd. for C₂₉H₃₈N₅O [M+H]⁺ m/z 472.31, found 472.25.

¹H NMR (400 MHz, DMSO-d₆, ppm) δ 8.30 (s, 1H), 8.20 (d, J=8.1 Hz, 1H), 7.42 (s, 1H), 7.11 (s, 4H), 6.42 (s, 2H), 4.82 (s, 1H), 4.54 (s, 1H), 4.01 (s, 2H), 3.13-2.98 (m, 3H), 2.94-2.68 (m, 3H), 2.13 (s, 1H), 1.91-1.68 (m, 4H), 1.48 (d, J=7.4 Hz, 2H), 1.21 (s, 6H), 1.00 (t, J=7.2 Hz, 3H).

### Example 31

### Synthesis of Compound 59

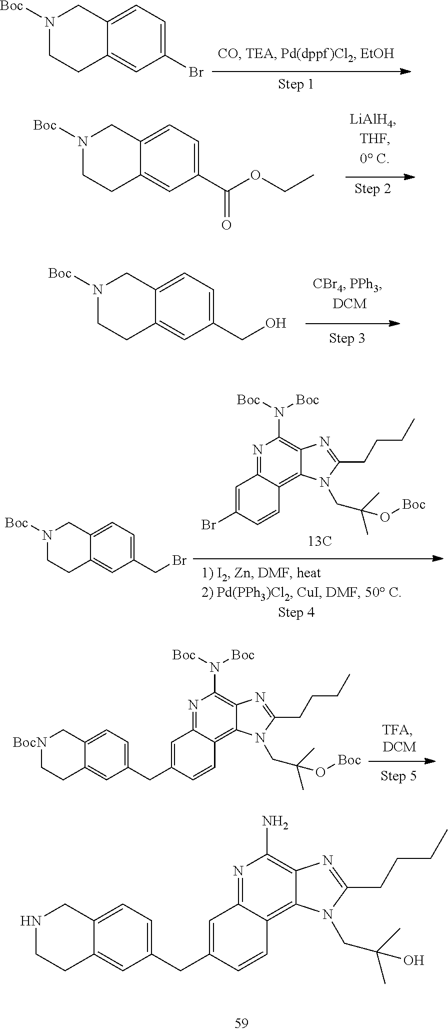

### Step 1. Preparation of 2-(tert-butyl) 6-ethyl 3,4-dihydroisoquinoline-2,6(1H)-dicarboxylate

A solution of tert-butyl 6-bromo-3,4-dihydroisoquinoline-2(1H)-carboxylate (3 g, 9.6 mmol), TEA (4.86 g, 48 mmol) and Pd(dppf)Cl₂ (0.35 g, 0.48 mmol) in EtOH (20 mL) was heated at 110° C. under an atmosphere of CO (30 atm) for 16 hours in a high-pressure reactor. LCMS showed the reaction was completed. The resulting solution was diluted with water (60 mL) and extracted with EtOAc (50 mL×3). The combined organic phases were washed with brine, dried over Na₂SO₄, concentrated under vacuum. The residue was purified by flash column chromatography on silica gel (PE/EtOAc=5/1) to give 2-(tert-butyl) 6-ethyl 3,4-dihydroisoquinoline-2,6(1H)-dicarboxylate (2.8 g, 89.6% yield).

LCMS (ESI) calcd. for C₁₇H₂₃NO₄ [M+H]⁺ m/z 306.17, found no MS.

### Step 2. Preparation of tert-butyl 6-(hydroxymethyl)-3,4-dihydroisoquinoline-2(1H)-carboxylate

To a solution of 2-(tert-butyl) 6-ethyl 3,4-dihydroisoquinoline-2,6(1H)-dicarboxylate (2.8 g, 9.1 mmol) in THF (20 mL) was added LiAlH₄ (1M in THF, 10 mL) dropwise. The mixture was stirred at 0° C. for 2 hours. LCMS showed the reaction was completed. The resulting solution was quenched with water and extracted with EtOAc (60 mL×3). The combined organic phases were washed with brine, dried over Na₂SO₄, concentrated under vacuum. The residue was purified by flash column chromatography on silica gel (PE/EtOAc=1/1) to give tert-butyl 6-(hydroxymethyl)-3,4-dihydroisoquinoline-2(1H)-carboxylate (2.4 g, 90.1% yield).

LCMS (ESI) calcd. for C₁₅H₂₁NO₃ [M+H]⁺ m/z 264.35, found no MS.

### Step 3. Preparation of tert-butyl 6-(bromomethyl)-3,4-dihydroisoquinoline-2(1H)-carboxylate

To a solution of tert-butyl 6-(hydroxymethyl)-3,4-dihydroisoquinoline-2(1H)-carboxylate (2.4 g, 9.1 mmol) and PPh₃ (3.1 g, 11.83 mmol) in DCM (20 mL) was added CBr₄ (3.92 g, 11.83 mmol). The mixture was stirred at room temperature for 1 hours. LCMS showed the reaction was completed. The resulting solution was concentrated under vacuum. The residue was purified by flash column chromatography on silica gel (PE/EtOAc=5/1) to give tert-butyl 6-(bromomethyl)-3,4-dihydroisoquinoline-2(1H)-carboxylate (1.7 g, 51.7% yield).

### Step 4. Preparation of tert-butyl 6-((4-(bis(tert-butoxycarbonyl)amino)-1-(2-((tert-butoxycarbonyl)oxy)-2-methylpropyl)-2-butyl-1H-imidazo[4,5-c]quinolin-7-yl)methyl)-3,4-dihydroisoquinoline-2(1H)-carboxylate

A mixture of Zn power (1.21 g, 18.6 mmol) and I₂ (250 mg, 1 mmol) in a 250 mL three-necked flask was evacuated and backfilled with N₂ three times and then charged with N₂. The flask was heated carefully with a hot air blower until I₂ was sublimated. Then a solution of tert-butyl 6-(bromomethyl)-3,4-dihydroisoquinoline-2(1H)-carboxylate (754 mg, 2.3 mmol) in DMF (5 mL) was added intermediately via a syringe. The mixture was stirred for 10 minutes while keeping the temperature was about 80° C. Then the heater and stirrer were removed. The upper layer clear solution was added dropwise into a stirred solution of tert-butyl (7-bromo-1-(2-((tert-butoxycarbonyl)oxy)-2-methylpropyl)-2-butyl-1H-imidazo[4,5-c]quinolin-4-yl)(tert-butoxycarbonyl)carbamate (400 mg, 0.58 mmol), CuI (90 mg, 0.45 mmol) and Pd(pph₃)₂Cl₂ (100 mg, 0.45 mmol) in DMF (3 mL) under an atmosphere of N₂. The mixture was heated at 50° C. for 1 hour. Then the mixture was quenched with water (50 mL) and extracted with EtOAc (50 mL×3). The combined organic phases were washed with brine, dried over Na₂SO₄, concentrated under vacuum. The residue was purified by flash column chromatography on silica gel (PE/EtOAc=2/1) to give tert-butyl 6-((4-(bis(tert-butoxycarbonyl)amino)-1-(2-((tert-butoxycarbonyl)oxy)-2-methylpropyl)-2-butyl-1H-imidazo[4,5-c]quinolin-7-yl)methyl)-3,4-dihydroisoquinoline-2(1H)-carboxylate (380 mg, 60.4% yield) as a yellow solid.

LCMS (ESI) calcd. for C₄₈H₆₈N₅O₉[M+H]⁺ m/z 858.50, found 858.45.

### Step 5. Preparation of 1-(4-amino-2-butyl-7-((1,2,3,4-tetrahydroisoquinolin-6-yl)methyl)-1H-imidazo[4,5-c]quinolin-1-yl)-2-methylpropan-2-ol (Compound 59)

To a solution of tert-butyl 6-((4-(bis(tert-butoxycarbonyl)amino)-1-(2-((tert-butoxycarbonyl)oxy)-2-methylpropyl)-2-butyl-1H-imidazo[4,5-c]quinolin-7-yl)methyl)-3,4-dihydroisoquinoline-2(1H)-carboxylate (380 mg, 0.44 mmol) in DCM (6 mL) was added TFA (2 mL) at room temperature. The reaction mixture was stirred at room temperature for 2 hours. After the reaction was completed, the mixture was concentrated. The residue was dissolved in water (30 mL) and adjusted to pH=8-9 with saturated aqueous NaHCO₃. The resulting solution was extracted with DCM (30 mL×3). The combined organic phases were washed with brine, dried over sodium sulfate, concentrated under vacuum. The residue was purified by prep-HPLC (Gemini 5 um C18 column, 150*21.2 mm, eluting with 15% to 75% MeCN/H₂O containing 0.1% FA) to provide 1-(4-amino-2-butyl-7-((1,2,3,4-tetrahydroisoquinolin-6-yl)methyl)-1H-imidazo[4,5-c]quinolin-1-yl)-2-methylpropan-2-ol (Compound 59) (56 mg, 26.4% yield) as a white solid.

LCMS (ESI) calcd. for C₂₈H₃₅N₅O [M+H]⁺ m/z 458.29, found 458.25.

¹H NMR (400 MHz, DMSO-d₆, ppm) δ 9.05 (s, 2H), 8.31 (d, J=8.5 Hz, 1H), 7.93 (s, 1H), 7.49 (s, 1H), 7.32-7.04 (m, 5H), 4.78 (s, 1H), 4.24 (d, J=6.4 Hz, 2H), 4.05 (s, 2H), 3.41-3.34 (m, 4H), 3.02-2.91 (m, 4H), 1.90-1.72 (m, 2H), 1.50-1.33 (m, 2H), 1.17 (s, 6H), 0.94 (t, J=7.3 Hz, 3H).

### Example 32

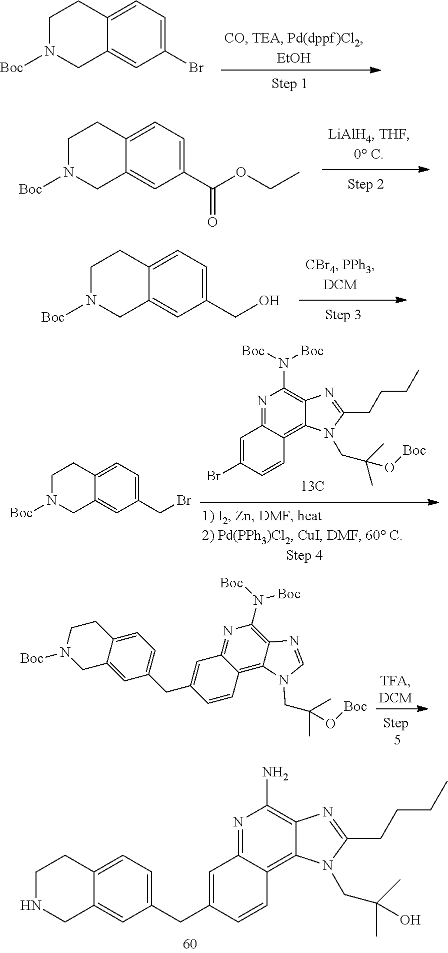

### Step 1: Preparation of 2-(tert-butoxycarbonyl)-1,2,3,4-tetrahydroisoquinoline-7-carboxylic acid

A solution of tert-butyl 7-bromo-3,4-dihydroisoquinoline-2(1H)-carboxylate (4 g, 12.86 mmol), TEA (6.49 g, 64.3 mmol) and Pd(dppf)Cl₂ (0.28 g, 0.39 mmol) in EtOH (30 mL) was heated at 100° C. under an atmosphere of CO (30 atm) for 16 hours in a high-pressure reactor. LCMS showed the reaction was completed. The resulting solution was diluted with water (60 mL) and extracted with EtOAc (50 mL×3). The combined organic phases were washed with brine, dried over Na₂SO₄, concentrated under vacuum. The residue was purified by flash column chromatography on silica gel (PE/EtOAc=1/3) to give 2-(tert-butoxycarbonyl)-1,2,3,4-tetrahydroisoquinoline-7-carboxylic acid (3.2 g, 81.6% yield).

LCMS (ESI) calcd. for C₁₇H₂₃NO₄ [M+H]⁺ m/z 306.17, found 306.12.

### Step 2: Preparation of tert-butyl 7-(hydroxymethyl)-3,4-dihydroisoquinoline-2(1H)-carboxylate

To a solution of 2-(tert-butyl) 7-ethyl 3,4-dihydroisoquinoline-2,7(1H)-dicarboxylate (3.2 g, 10.49 mmol) in THF (10 mL) was added LiAlH₄ (1 M in THF, 5 mL) dropwise. The mixture was stirred at 0° C. for 1 hours. LCMS showed the reaction was completed. The resulting solution was quenched with water and extracted with EtOAc (60 mL×3). The combined organic phases were washed with brine, dried over Na₂SO₄, concentrated under vacuum. The residue was purified by flash column chromatography on silica gel (PE/EtOAc=1/1) to give tert-butyl 7-(hydroxymethyl)-3,4-dihydroisoquinoline-2(1H)-carboxylate (2.5 g, 90.61% yield).

LCMS (ESI) calcd. for C₁₅H₂₁NO₃ [M+H -Boc]⁺ m/z 286.16, found 285.90.

### Step 3: Preparation of tert-butyl (7-(bromomethyl)-1,2,3,4-tetrahydronaphthalen-1-yl)carbamate

To a solution of tert-butyl 7-(hydroxymethyl)-3,4-dihydroisoquinoline-2(1H)-carboxylate (2.5 g, 9.5 mmol) and PPh₃ (2.99 g, 11.4 mmol) in DCM (8 mL) was added CBr₄ (3.77 g, 11.4 mmol). The mixture was stirred at room temperature for 1 hours. LCMS showed the reaction was completed. The resulting solution was concentrated under vacuum. The residue was purified by flash column chromatography on silica gel (PE/EtOAc=5/1) to give tert-butyl 7-(bromomethyl)-3,4-dihydroisoquinoline-2(1H)-carboxylate (1.5 g, 48.7% yield).

LCMS (ESI) calcd. for C₁₅H₂₀BrNO₂ [M+H−Boc]⁺ m/z 270.08, found 269.85.

### Step 4: Preparation of tert-butyl 7-((4-(bis(tert-butoxycarbonyl)amino)-1-(2-((tert-butoxycarbonyl)oxy)-2-methylpropyl)-2-butyl-1H-imidazo[4,5-c]quinolin-7-yl)methyl)-3,4-dihydroisoquinoline-2(1H)-carboxylate

A mixture of Zn power (640 mg, 10.01 mmol) and I₂ (200 mg, 0.79 mmol) in a 250 mL three-necked flask was evacuated and backfilled with N₂ three times and then charged with N₂. The flask was heated carefully with a hot air blower until I₂ was sublimated. Then a solution of tert-butyl 7-(bromomethyl)-3,4-dihydroisoquinoline-2(1H)-carboxylate (500 mg, 1.54 mmol) in DMF (5 mL) was added intermediately via a syringe. The mixture was stirred for 10 minutes while keeping the temperature was about 80° C. Then the heater and stirrer were removed. The upper layer clear solution was added dropwise into a stirred solution of tert-butyl (7-bromo-1-(2-((tert-butoxycarbonyl)oxy)-2-methylpropyl)-2-butyl-1H-imidazo[4,5-c]quinolin-4-yl)(tert-butoxycarbonyl) carbamate (200 mg, 0.29 mmol), CuI (11 mg, 0.06 mmol) and Pd(pph₃)₂Cl₂ (21 mg, 0.03 mmol) in DMF (3 mL) under an atmosphere of N₂. The mixture was heated at 50° C. for 1 hour. Then the mixture was quenched with water (50 mL) and extracted with EtOAc (50 mL×3). The combined organic phases were washed with brine, dried over Na₂SO₄, concentrated under vacuum. The residue was purified by flash column chromatography on silica gel (PE/EtOAc=2/1) to give tert-butyl 7-((4-(bis(tert-butoxycarbonyl)amino)-1-(2-((tert-butoxycarbonyl)oxy)-2-methylpropyl)-2-butyl-1H-imidazo[4,5-c]quinolin-7-yl)methyl)-3,4-dihydroisoquinoline-2(1H)-carboxylate (150 mg, 60.48% yield) as a yellow solid.

LCMS (ESI) calcd. for C₄₈H₆₇N₅O₉ [M+H]⁺ m/z 858.50, found 858.45.

### Step 5: Preparation of 1-(4-amino-2-butyl-7-((1,2,3,4-tetrahydroisoquinolin-7-yl)methyl)-1H-imidazo[4,5-c]quinolin-1-yl)-2-methylpropan-2-ol (Compound 60)

To a solution of tert-butyl 7-((4-(bis(tert-butoxycarbonyl)amino)-1-(2-((tert-butoxycarbonyl)oxy)-2-methylpropyl)-2-butyl-1H-imidazo[4,5-c]quinolin-7-yl)methyl)-3,4-dihydroisoquinoline-2(1H)-carboxylate (150 mg, 0.18 mmol) in DCM (1.5 mL) was added TFA (0.5 mL) at room temperature. The reaction mixture was stirred at room temperature for 2 hours. After the reaction was completed, the mixture was concentrated. The residue was dissolved in water and adjusted to pH=8-9 with saturated aqueous NaHCO₃. The resulting solution was extracted with DCM (10 mL×3). The combined organic phases were washed with brine, dried over sodium sulfate, concentrated under vacuum. The residue was purified by prep-HPLC (Gemini 5 um C18 column, 150*21.2 mm, eluting with 40% to 85% MeCN/H₂O containing 0.1% TFA) to provide 1-(4-amino-2-butyl-7-((1,2,3,4-tetrahydroisoquinolin-7-yl)methyl)-1H-imidazo[4,5-c]quinolin-1-yl)-2-methylpropan-2-ol (Compound 60) (35 mg, 43.75% yield) as a white solid.

LCMS (ESI) calcd. for C₂₈H₃₅N₅O [M+H]⁺ m/z 458.29, found 458.15.

¹H NMR (400 MHz, DMSO-d₆, ppm) δ 14.05 (s, 1H), 9.19 (s, 2H), 9.00 (s, 2H), 8.41 (d, J=8.6 Hz, 1H), 7.57 (s, 1H), 7.32 (d, J=8.6 Hz, 1H), 7.23-7.15 (m, 2H), 7.13 (s, 1H), 4.70 (s, 2H), 4.24 (s, 2H), 4.09 (s, 2H), 3.36 (s, 2H), 3.00 (dt, J=12.1, 6.8 Hz, 4H), 1.89-1.72 (m, 2H), 1.51-1.36 (m, 2H), 1.18 (s, 6H), 0.94 (t, J=7.3 Hz, 3H).

### Example 33

### Synthesis of Compound 61

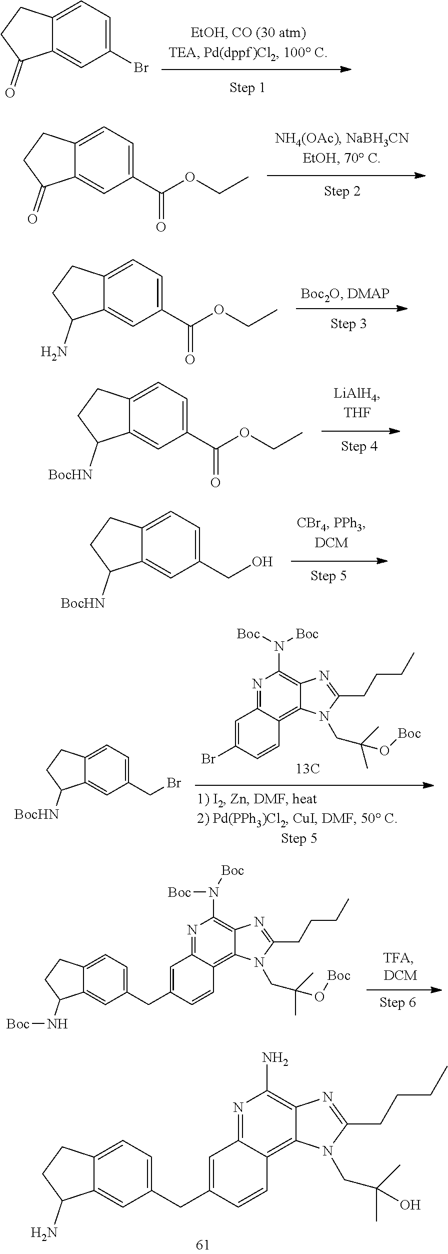

### Step 1: Preparation of ethyl 3-oxo-2,3-dihydro-1H-indene-5-carboxylate

A solution of 7-bromo-3,4-dihydronaphthalen-1(2H)-one (4 g, 19.04 mmol), TEA (13.46 g, 133.28 mmol) and Pd(dppf)Cl₂ (0.69 g, 0.95 mmol) in EtOH (30 mL) was heated at 100° C. under an atmosphere of CO (30 atm) for 16 hours in a high-pressure reactor. LCMS showed the reaction was completed. The resulting solution was diluted with water (60 mL) and extracted with EtOAc (50 mL×3). The combined organic phases were washed with brine, dried over Na₂SO₄, concentrated under vacuum. The residue was purified by flash column chromatography on silica gel (PE/EtOAc=1/3) to give ethyl 3-oxo-2,3-dihydro-1H-indene-5-carboxylate (2.5 g, 46.4% yield).

LCMS (ESI) calcd. for C₁₂H₁₃O₃[M+H]⁺ m/z 205.09, found 205.10.

### Step 2: Preparation of ethyl 3-amino-2,3-dihydro-1H-indene-5-carboxylate

A solution of ethyl 3-oxo-2,3-dihydro-1H-indene-5-carboxylate (2.5 g, 12.2 mmol), NH₄(OAc) (14.11 g, 183.24 mmol) and NaBH₃CN (3.83 g, 60.79 mmol) in EtOH (50 mL) was heated at 70° C. for 4 hours. LCMS showed the reaction was completed. The resulting solution was concentrated under vacuum and purified by flash column chromatography on silica gel (PE/EtOAc=1/5) to give ethyl 3-amino-2,3-dihydro-1H-indene-5-carboxylate (2 g, 72.1% yield).

LCMS (ESI) calcd. for C₁₂H₁₆NO₂ [M+H]⁺ m/z 206.12, found 206.05.

### Step 3: Preparation of ethyl 3-((tert-butoxycarbonyl)amino)-2,3-dihydro-1H-indene-5-carboxylate

A solution of ethyl 3-amino-2,3-dihydro-1H-indene-5-carboxylate (2 g, 9.70 mmol), Boc₂O (4.23 g, 19.40 mmol) and DMAP (0.47 g, 3.88 mmol) in MeCN (30 mL) was heated at 75° C. 3 hours. LCMS showed the reaction was completed. The resulting solution was diluted with water (100 mL) and extracted with EtOAc (60 mL×3). The combined organic phases were washed with brine, dried over Na₂SO₄, concentrated under vacuum. The residue was purified by flash column chromatography on silica gel (PE/EtOAc=2/1) to give ethyl 3-(bis(tert-butoxycarbonyl)amino)-2,3-dihydro-1H-indene-5-carboxylate (1.5 g, 34.0% yield).

LCMS (ESI) calcd. for C₁₇H₂₄NO₆ Na [M+H]⁺ m/z 306.17, found [M+H+Na]⁺ m/z 328.16.

### Step 4: Preparation of tert-butyl (6-(hydroxymethyl)-2,3-dihydro-1H-inden-1-yl)carbamate

To a solution of ethyl 3-((tert-butoxycarbonyl)amino)-2,3-dihydro-1H-indene-5-carboxylate (1.5 g, 4.90 mmol) in THF (15 mL) was added LiAlH₄ (1 M in THF, 7.5 mL) dropwise. The mixture was stirred at room temperature for 1 hours. LCMS showed the reaction was completed. The resulting solution was quenched with water and extracted with EtOAc (60 mL×3). The combined organic phases were washed with brine, dried over Na₂SO₄, concentrated under vacuum. The residue was purified by flash column chromatography on silica gel (PE/EtOAc=1/1) to give tert-butyl (6-(hydroxymethyl)-2,3-dihydro-1H-inden-1-yl)carbamate (1.2 g, 83.6% yield).

LCMS (ESI) calcd. for C₁₅H₂₂NO₃Na [M+H]⁺ m/z 264.16, found [M+H+Na]⁺ m/z 286.15.

### Step 5: Preparation of tert-butyl (6-(bromomethyl)-2,3-dihydro-1H-inden-1-yl)carbamate

To a solution of tert-butyl (6-(hydroxymethyl)-2,3-dihydro-1H-inden-1-yl)carbamate (1.2 g, 4.54 mmol) and PPh₃ (1.42 g, 5.39 mmol) in DCM (8 mL) was added CBr₄ (1.79 g, 5.39 mmol) at 0° C. The mixture was stirred at room temperature for 1 hours. LCMS showed the reaction was completed. The resulting solution was concentrated under vacuum. The residue was purified by flash column chromatography on silica gel (PE/EtOAc=5/1) to give tert-butyl (6-(bromomethyl)-2,3-dihydro-1H-inden-1-yl)carbamate (500 mg, 31.1% yield).

LCMS (ESI) calcd. for C₁₅H₂₁BrNO₂ [M+H]⁺ m/z 326.08, found [M+H -tBu]⁺ m/z 270.05.

### Step 6: Preparation of tert-butyl (tert-butoxycarbonyl)(7-((3-((tert-butoxycarbonyl)amino)-2,3-dihydro-1H-inden-5-yl)methyl)-1-(2-((tert-butoxycarbonyl)oxy)-2-methylpropyl)-2-butyl-1H-imidazo[4,5-c]quinolin-4-yl)carbamate

A mixture of Zn power (1 g, 15.27 mmol) and I₂ (310 mg, 1.22 mmol) in a 250 mL three-necked flask was evacuated and backfilled with N₂ three times and then charged with N₂. The flask was heated carefully with a hot air blower until I₂ was sublimated. Then a solution of tert-butyl (6-(bromomethyl)-2,3-dihydro-1H-inden-1-yl)carbamate (500 mg, 1.52 mmol) in DMF (5 mL) was added intermediately via a syringe. The mixture was stirred for 10 minutes while keeping the temperature was about 80° C. Then the heater and stirrer were removed. The upper layer clear solution was added dropwise into a stirred solution of tert-butyl (7-bromo-1-(2-((tert-butoxycarbonyl)oxy)-2-methylpropyl)-2-butyl-1H-imidazo[4,5-c]quinolin-4-yl)(tert-butoxycarbonyl) carbamate (300 mg, 0.45 mmol), CuI (58 mg, 0.30 mmol) and Pd(pph₃)₂Cl₂ (107 mg, 0.15 mmol) in DMF (3 mL) under an atmosphere of N₂. The mixture was heated at 50° C. for 1 hour. Then the mixture was quenched with water (50 mL) and extracted with EtOAc (50 mL×3). The combined organic phases were washed with brine, dried over sodium sulfate and concentrated under vacuum. The residue was purified by flash column chromatography on silica gel (PE/EtOAc=2/1) to provide tert-butyl (tert-butoxycarbonyl)(7-((3-((tert-butoxycarbonyl)amino)-2,3-dihydro-1H-inden-5-yl)methyl)-1-(2-((tert-butoxycarbonyl)oxy)-2-methylpropyl)-2-butyl-1H-imidazo[4,5-c]quinolin-4-yl)carbamate (300 mg, 20.5% yield) as a yellow solid.

LCMS (ESI) calcd. for C₄₈H₆₈N₅O₉[M+H]⁺ m/z 858.50, found 858.45.

### Step 7: Preparation of 1-(4-amino-7-((3-amino-2,3-dihydro-1H-inden-5-yl)methyl)-2-butyl-1H-imidazo[4,5-c]quinolin-1-yl)-2-methylpropan-2-ol (Compound 61)

To a solution of tert-butyl (tert-butoxycarbonyl)(7-((3-((tert-butoxycarbonyl)amino)-2,3-dihydro-1H-inden-5-yl)methyl)-1-(2-((tert-butoxycarbonyl)oxy)-2-methylpropyl)-2-butyl-1H-imidazo[4,5-c]quinolin-4-yl) carbamate (300 mg, 0.34 mmol) in DCM (1.5 mL) was added TFA (0.5 mL) at room temperature. The reaction mixture was stirred at room temperature for 2 hours. After the reaction was completed, the mixture was concentrated. The residue was dissolved water and then adjusted to pH=8-9 with saturated aqueous NaHCO₃. The resulting solution was extracted with DCM (10 mL×3). The combined organic phases were washed with brine, dried over sodium sulfate, concentrated under vacuum. The residue was purified by prep-HPLC (Gemini 5 um C18 column, 150*21.2 mm, eluting with 20% to 30% MeCN/H₂O containing 0.1% FA) to provide 1-(4-amino-7-((3-amino-2,3-dihydro-1H-inden-5-yl)methyl)-2-butyl-1H-imidazo[4,5-c]quinolin-1-yl)-2-methylpropan-2-ol (Compound 61) (100 mg, 56.5% yield) as a white solid.

¹H NMR (400 MHz, DMSO-d6, ppm) δ 8.35 (d, J=8.6 Hz, 1H), 8.26 (s, 2H), 8.15 (s, 1H), 7.53 (s, 1H), 7.44 (s, 1H), 7.33-7.22 (m, 3H), 4.80 (s, 1H), 4.71-4.62 (m, 2H), 4.17-4.04 (m, 2H), 3.02 (dd, J=16.5, 8.7 Hz, 3H), 2.90-2.74 (m, 1H), 2.48-2.39 (m, 1H), 1.95 (dt, J=14.0, 7.0 Hz, 1H), 1.85-1.75 (m, 2H), 1.48-1.37 (m, 2H), 1.17 (s, 6H), 0.94 (t, J=7.3 Hz, 3H).

LCMS (ESI) calcd. for C₂₈H₃₆N₅O [M+H]⁺ m/z 458.29, found 458.25.

### Example 34

### Synthesis of Compound 62

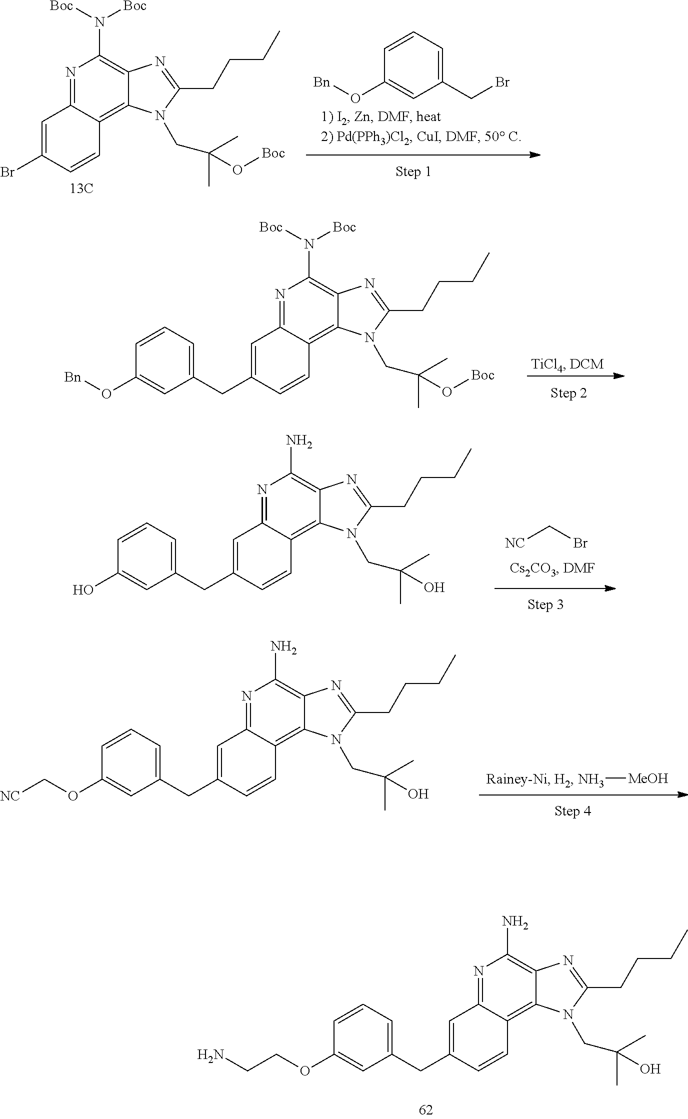

### Step 1: tert-butyl (7-(3-(benzyloxy)benzyl)-1-(2-((tert-butoxycarbonyl)oxy)-2-methylpropyl)-2-butyl-1H-imidazo[4,5-c]quinolin-4-yl)(tert-butoxycarbonyl)carbamate

A mixture of Zn power (2.4 g, 37 mmol) and I₂ (182.69 mg, 0.72 mmol) in a 100 mL three-necked flask was evacuated and backfilled with N₂ three times and then charged with N₂. The flask was heated carefully with a hot air blower until all I₂ was sublimated. Then a solution of 1-(bromomethyl)-3-(1-methylphenoxy)benzene (1.6 g, 5.8 mmol) in DMF (8 mL) was added intermediately via a syringe. The mixture was stirred for 10 minutes while keeping the temperature was about 70° C. Then the heater and stirrer were removed. The upper layer clear solution was added dropwise into a stirred solution of tert-butyl (7-bromo-1-(2-((tert-butoxycarbonyl)oxy)-2-methylpropyl)-2-butyl-1H-imidazo[4,5-c]quinolin-4-yl)(tert-butoxycarbonyl) carbamate 13C (400 mg, 0.58 mmol), Copper(I) iodide (22.2 mg, 0.11 mmol) and Pd (PPh₃)₂ Cl₂ (81.85 mg, 0.11 mmol) in DMF (2 mL) under an atmosphere of N₂. The mixture was heated at 50° C. for 1 hour. Then the mixture was quenched with water (50 mL) and extracted with EtOAc (50 mL×3). The combined organic phases were washed with water and brine, dried over Na₂SO₄ and concentrated under vacuum. The residue was purified by flash column chromatography on silica gel (PE/EtOAc=4:1) to afford tert-butyl (7-(3-(benzyloxy)benzyl)-1-(2-((tert-butoxycarbonyl)oxy)-2-methylpropyl)-2-butyl-1H-imidazo[4,5-c]quinolin-4-yl)(tert-butoxycarbonyl)carbamate (350 mg, 74% yield) as a yellow solid.

LCMS (ESI) calcd for C₄₇H₆₀N₄O₈ [M+H]⁺ ms/z=809.45, found 809.45.

### Step 2: 3-((4-amino-2-butyl-1-(2-hydroxy-2-methylpropyl)-1H-imidazo[4,5-c]quinolin-7-yl)methyl)phenol

To a solution of tert-butyl (7-(3-(benzyloxy)benzyl)-1-(2-((tert-butoxycarbonyl)oxy)-2-methylpropyl)-2-butyl-1H-imidazo[4,5-c]quinolin-4-yl)(tert-butoxycarbonyl)carbamate (300 mg, 0.37 mmol) in DCM (8 mL) was added a solution of TiCl₄ (69.99 mg, 0.37 mmol) in DCM (2 mL) dropwise at 0° C. The reaction mixture was stirred at 0° C. for 30 minutes. Then the mixture was quenched with water (40 mL) and adjusted the pH=7 with NaHCO₃ saturated solution. The mixture was extracted with DCM (30 mL×3). The combined organic layers were washed with brine, dried over Na₂SO₄, filtered, and concentrated to give the crude. The residue was purified by silica gel chromatography (DCM/MeOH=20:1) to afford 3-((4-amino-2-butyl-1-(2-hydroxy-2-methylpropyl)-1H-imidazo[4,5-c]quinolin-7-yl)methyl)phenol (120 mg, 77.69% yield) as a yellow solid.

LCMS (ESI) calcd for C₂₅H₃₀N₄O₂ [M+H]⁺ ms/z=419.24, found 419.24.

### Step 3: 2-(3-((4-amino-2-butyl-1-(2-hydroxy-2-methylpropyl)-1H-imidazo[4,5-c]quinolin-7-yl)methyl) phenoxy)acetonitrile

To a solution of 3-((4-amino-2-butyl-1-(2-hydroxy-2-methylpropyl)-1H-imidazo[4,5-c]quinolin-7-yl)methyl)phenol (100 mg, 0.24 mmol), Cs₂CO₃ (234 mg, 0.72 mmol) in DMF (5 mL) was added 2-bromoacetonitrile (570.86 mg, 4.76 mmol) dropwise at 25° C. The reaction mixture was stirred at 25° C. for 3 hours. The mixture was quenched with water (60 mL) and extracted with EtOAc (40 mL×3). The combined organic layers were washed with brine, dried over Na₂SO₄, filtered, concentrated to give the crude. The reside was purified by silica gel chromatography (DCM/MeOH=20:1) to afford 3-((4-amino-2-butyl-1-(2-hydroxy-2-methylpropyl)-1H-imidazo[4,5-c]quinolin-7-yl)methyl)phenol (100 mg, 76% yield) as a yellow solid.

LCMS (ESI) calcd for C₂₇H₃₁N₅O₂ [M+H]⁺ ms/z=458.26, found 458.26.

### Step 4: 1-(4-amino-7-(3-(2-aminoethoxy)benzyl)-2-butyl-1H-imidazo[4,5-c]quinolin-1-yl)-2-methylpropan-2-ol (Compound 62)

To a solution of 2-(3-((4-amino-2-butyl-1-(2-hydroxy-2-methylpropyl)-1H-imidazo[4,5-c]quinolin-7-yl)methyl)phenoxy)acetonitrile (70 mg, 0.15 mmol) in NH₃-MeOH (5 mL) at 25° C. was added a suspension of Raney Ni in NH₃-MeOH (1 mL). The reaction mixture was stirred at RT for 1 hour under hydrogen. LCMS showed the reaction was completed. Then the mixture was filtered through celite and concentrated under vacuo to give the crude. The residue was purified by Prep-HPLC (Gemini 5 um C18 column, 150*21.2 mm, eluting with 20% to 70% MeCN/H₂O containing 0.1% FA) to afford 1-(4-amino-7-(3-(2-aminoethoxy)benzyl)-2-butyl-1H-imidazo[4,5-c]quinolin-1-yl)-2-methylpropan-2-ol (23 mg, 32.23% yield) as a white solid.

LCMS (ESI) calcd for C₂₇H₃₅N₅O₂ [M+H]⁺ ms/z=462.29, found 462.25.

1H NMR (400 MHz, DMSO-d₆, ppm) δ 13.36 (s, 1H), 8.73 (s, 2H), 8.42 (d, J=8.7 Hz, 1H), 7.92 (s, 3H), 7.57 (s, 1H), 7.36 (d, J=8.5 Hz, 1H), 7.29 (t, J=7.9 Hz, 1H), 6.97-6.81 (m, 3H), 4.80 (s, 1H), 4.52 (s, 2H), 4.16-4.05 (m, 4H), 3.21 (d, J=4.4 Hz, 2H), 3.04 (t, J=7.7 Hz, 2H), 1.89-1.73 (m, 2H), 1.43 (dd, J=15.0, 7.4 Hz, 2H), 1.18 (s, 6H), 0.94 (t, J=7.3 Hz, 3H).

### Example 35

### Synthesis of Compound 63

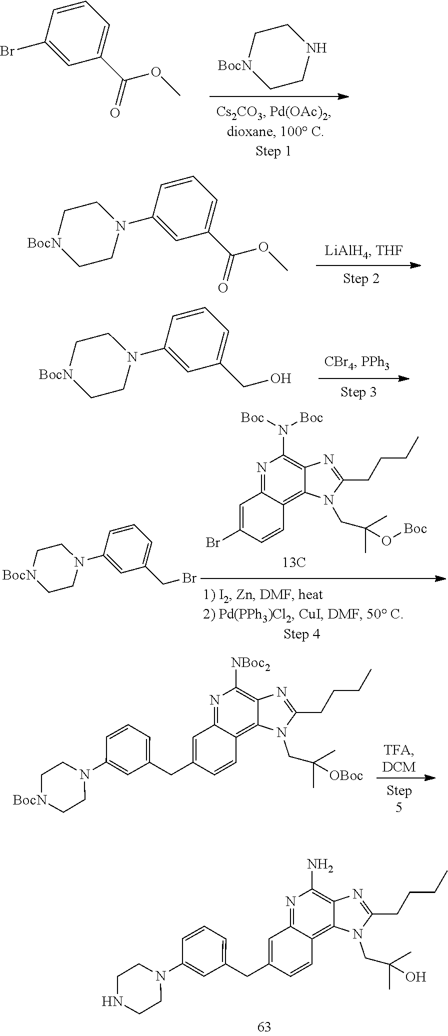

### Step 1: Preparation of tert-butyl 4-(3-(methoxycarbonyl)phenyl)piperazine-1-carboxylate

A solution of methyl 3-bromobenzoate (5 g, 23.36 mmol), tert-butyl piperazine-1-carboxylate (4.34 g, 23.36 mmol), Cs₂CO₃ (22.84 g, 70.08 mmol), Xantphos (2.02 g, 3.50 mmol) and Pd(OAc)₂ (0.42 g, 1.86 mmol) in 1,4-dioxane (50 mL) was stirred at 100° C. for 16 h under N₂ atmosphere. The resulting solution was quenched with water (200 mL) and extracted with EtOAc (80 mL×3). The combined organic phases were washed with brine, dried over Na₂SO₄, concentrated under vacuum. The residue was purified by flash column chromatography on silica gel (PE/EtOAc=2/1) to give tert-butyl 4-(3-(methoxycarbonyl)phenyl)piperazine-1-carboxylate (4.8 g, 64.0% yield).

LCMS (ESI) calcd. for C₁₇H₂₅N₂O₄ [M+H]⁺ m/z 321.18, found 321.05.

### Step 2: Preparation of tert-butyl 4-(3-(hydroxymethyl)phenyl)piperazine-1-carboxylate

To a solution of tert-butyl 4-(3-(methoxycarbonyl)phenyl)piperazine-1-carboxylate (2.5 g, 7.79 mmol) in THF (30 mL) was added LiAlH₄ (1M in THF, 11 mL) dropwise. The mixture was stirred at room temperature for 1 hours. LCMS showed the reaction was completed. The reaction was quenched with water and extracted with EtOAc (80 mL×3). The combined organic phases were washed with brine, dried over Na₂SO₄, concentrated under vacuum. The resulting solution was concentrated under vacuum. The residue was purified by flash column chromatography on silica gel (PE/EtOAc=1/1) to give tert-butyl 4-(3-(hydroxymethyl)phenyl)piperazine-1-carboxylate (1.4 g, 61.3% yield).

LCMS (ESI) calcd. for C₁₆H₂₅N₂O₃ [M+H]⁺ m/z 293.19, found 293.20.

### Step 3: Preparation of tert-butyl 4-(3-(bromomethyl)phenyl)piperazine-1-carboxylate

To a solution of tert-butyl 4-(3-(hydroxymethyl)phenyl)piperazine-1-carboxylate (1.3 g, 4.4 mmol) and PPh₃ (1.38 g, 5.28 mmol) in DCM (10 mL) was added CBr₄ (1.75 g, 5.28 mmol) at 0° C. The mixture was stirred at RT for 1 hours. LCMS showed the reaction was completed. The resulting solution was concentrated under vacuum. The residue was purified by flash column chromatography on silica gel (PE/EtOAc=5/1) to give tert-butyl 4-(3-(bromomethyl)phenyl)piperazine-1-carboxylate (600 mg, 38.4% yield).

LCMS (ESI) calcd. for C₁₆H₂₄BrN₂O₂[M+H]⁺ m/z 355.10, found 355.10.

### Step 4: Preparation of tert-butyl 4-(3-((4-(bis(tert-butoxycarbonyl)amino)-1-(2-((tert-butoxycarbonyl)oxy)-2-methylpropyl)-2-butyl-1H-imidazo[4,5-c]quinolin-7-yl)methyl)phenyl)piperazine-1-carboxylate

A mixture of Zn power (1.3 g, 19.69 mmol) and I₂ (214 mg, 0.84 mmol) in a 250 mL three-necked flask was evacuated and backfilled with N₂ three times and then charged with N₂. The flask was heated carefully with a hot air blower until I₂ was sublimated. Then a solution of tert-butyl 4-(3-(bromomethyl)phenyl)piperazine-1-carboxylate (600 mg, 1.68 mmol) in DMF (5 mL) was added intermediately via a syringe. The mixture was stirred for 10 minutes while keeping the temperature was about 80° C. Then the heater and stirrer were removed. The upper layer clear solution was added dropwise into a stirred solution tert-butyl (7-bromo-1-(2-((tert-butoxycarbonyl)oxy)-2-methylpropyl)-2-butyl-1H-imidazo[4,5-c]quinolin-4-yl)(tert-butoxycarbonyl) carbamate (200 mg, 0.29 mmol), CuI (27 mg, 0.14 mmol) and Pd(pph₃)₂Cl₂ (40 mg, 0.05 mmol) in DMF (3 mL) under an atmosphere of N₂. The mixture was heated at 50 TC for 1 hour. Then the mixture was quenched with water (50 mL) and extracted with EtOAc (50 mL×3). The combined organic phases were washed with brine, dried over sodium sulfate and concentrated under vacuum. The residue was purified by flash column chromatography on silica gel (PE/EtOAc=2/1) to give tert-butyl 4-(3-((4-(bis(tert-butoxycarbonyl)amino)-1-(2-((tert-butoxycarbonyl)oxy)-2-methylpropyl)-2-butyl-1H-imidazo[4,5-c]quinolin-7-yl)methyl)phenyl)piperazine-1-carboxylate (150 mg, 55.8% yield) as a yellow solid.

LCMS (ESI) calcd. for C₄₉H₇₁N₆O₉ [M+H]⁺ m/z 887.53, found 887.45.

### Step 5: Preparation of 1-(4-amino-2-butyl-7-(3-(piperazin-1-yl)benzyl)-1H-imidazo[4,5-c]quinolin-1-yl)-2-methylpropan-2-ol (Compound 63)

To a solution of tert-butyl 4-(3-((4-(bis(tert-butoxycarbonyl)amino)-1-(2-((tert-butoxycarbonyl)oxy)-2-methylpropyl)-2-butyl-1H-imidazo[4,5-c]quinolin-7-yl)methyl)phenyl)piperazine-1-carboxylate (150 mg, 0.17 mmol) in DCM (1.5 mL) was added TFA (0.5 mL) at room temperature. The reaction mixture was stirred at room temperature for 2 hours. After the reaction was completed, the mixture was concentrated. The residue was dissolved in THF (2 mL) then adjusted to pH=8-9 with saturated aqueous NaHCO₃. The resulting solution was extracted with DCM (10 mL×3). The combined organic phases were washed with brine, dried over sodium sulfate, concentrated under vacuum. The residue was purified by prep-HPLC (Gemini 5 um C18 column, 150*21.2 mm, eluting with 10% to 75% MeCN/H₂O containing 0.1% FA) to provide 1-(4-amino-2-butyl-7-(3-(piperazin-1-yl)benzyl)-1H-imidazo[4,5-c]quinolin-1-yl)-2-methylpropan-2-ol (Compound 63) (30 mg, 36.3% yield) as a white solid.

¹H NMR (400 MHz, DMSO-d₆, ppm) δ 8.33 (d, J=8.5 Hz, 1H), 8.15 (s, 1H), 7.51 (s, 1H), 7.27 (d, J=8.5 Hz, 1H), 7.20 (t, J=7.8 Hz, 1H), 6.96 (s, 1H), 6.88-6.81 (m, 1H), 6.78 (d, J=7.4 Hz, 1H), 4.79 (s, 1H), 4.04 (s, 2H), 3.38-3.28 (m, 6H), 3.23 (d, J=5.2 Hz, 4H), 3.03 (t, J=7.7 Hz, 2H), 1.88-1.72 (m, 2H), 1.42 (dd, J=14.9, 7.4 Hz, 2H), 1.17 (s, 6H), 0.94 (t, J=7.3 Hz, 3H).

LCMS (ESI) calcd. for C₂₉H₃₉N₆O [M+H]⁺ m/z 487.32, found 487.30.

### Example 36

### Synthesis of Compound 64

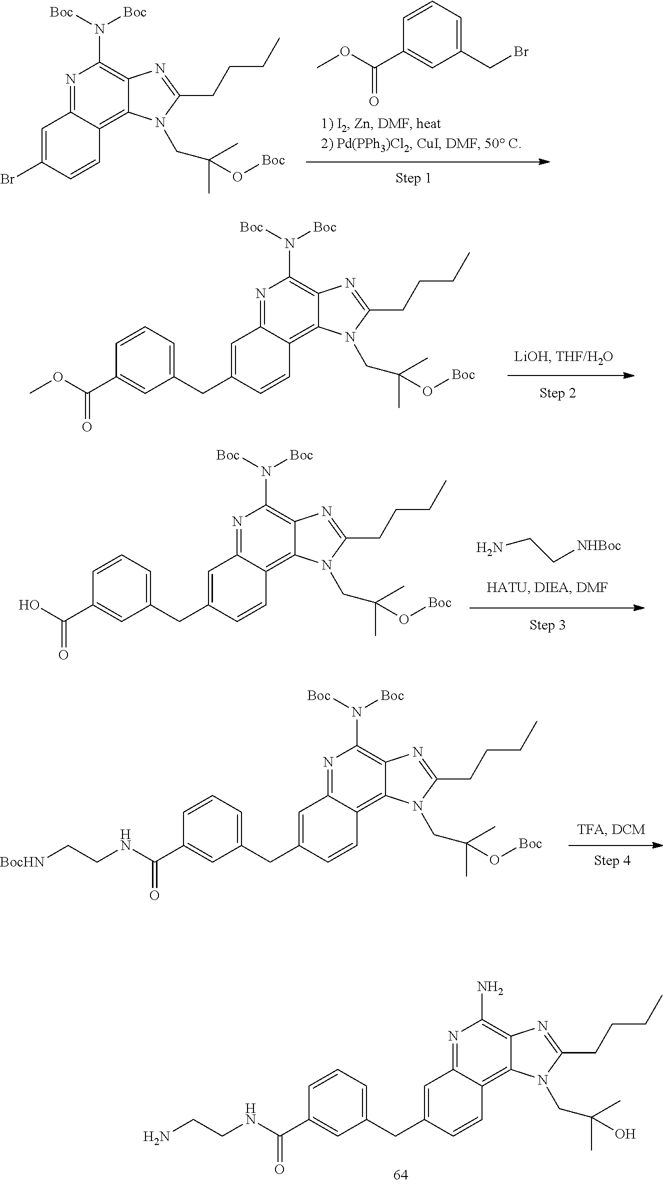

### Step 1: methyl 3-((4-(bis(tert-butoxycarbonyl)amino)-1-(2-((tert-butoxycarbonyl)oxy)-2-methylpropyl)-2-butyl-1H-imidazo[4,5-c]quinolin-7-yl)methyl)benzoate

A mixture of Zn power (2.4 g, 37 mmol) and I₂ (182.69 mg, 0.72 mmol) in a 100 mL three-necked flask was evacuated and backfilled with N₂ three times and then charged with N₂. The flask was heated carefully with a hot air blower until all I₂ was sublimated. Then a solution of methyl 3-(bromomethyl) benzoate (1 g, 4.37 mmol) in DMF (8 mL) was added intermediately via a syringe. The mixture was stirred for 10 minutes while keeping the temperature was about 70° C. Then the heater and stirrer were removed. The upper layer clear solution was added dropwise into a stirred solution of tert-butyl (7-bromo-1-(2-((tert-butoxycarbonyl)oxy)-2-methylpropyl)-2-butyl-1H-imidazo[4,5-c]quinolin-4-yl)(tert-butoxycarbonyl)carbamate (300 mg, 0.43 mmol), Copper(I) iodide (16.45 mg, 0.086 mmol) and Pd (PPh₃)₂ Cl₂(60.63 mg, 0.86 mmol) in DMF (3 mL) under an atmosphere of N₂. The mixture was heated at 50° C. for 1 hour. Then the mixture was quenched with water (50 mL) and extracted with EtOAc (50 mL×3). The combined organic layers were washed with water and brine, dried over Na₂SO₄, and concentrated under vacuum. The residue was purified by flash column chromatography on silica gel (PE/EtOAc=2:1) to afford methyl 3-((4-(bis(tert-butoxycarbonyl)amino)-1-(2-((tert-butoxycarbonyl)oxy)-2-methylpropyl)-2-butyl-1H-imidazo[4,5-c]quinolin-7-yl)methyl)benzoate (250 mg, 75.77% yield) as a yellow solid.

LCMS (ESI) calcd for C₄₂H₅₆N₄O₉ [M+H]⁺ ms/z=761.41, found 761.40.

### Step 2: 3-((4-(bis(tert-butoxycarbonyl)amino)-1-(2-((tert-butoxycarbonyl)oxy)-2-methylpropyl)-2-butyl-1H-imidazo[4,5-c]quinolin-7-yl)methyl)benzoic acid

To a solution of methyl 3-((4-(bis(tert-butoxycarbonyl)amino)-1-(2-((tert-butoxycarbonyl)oxy)-2-methylpropyl)-2-butyl-1H-imidazo[4,5-c]quinolin-7-yl)methyl)benzoate (250 mg, 0.33 mmol) in (THF/H₂O=1/1, 6 mL) was added lithium hydroxide (78.38 mg, 3.27 mmol) at 25° C. The reaction mixture was stirred at RT for 8 hours. The mixture was quenched with water (50 mL) and adjusted pH=7 with 1N HCl. The mixture was extracted with EtOAc (40 mL×3). The combined organic layers were washed with brine, dried over Na₂SO₄ and concentrated under vacuum. The residue was purified by flash column chromatography on silica gel (PE/EtOAc=1:1) to afford 3-((4-(bis(tert-butoxycarbonyl)amino)-1-(2-((tert-butoxycarbonyl) oxy)-2-methylpropyl)-2-butyl-1H-imidazo[4,5-c]quinolin-7-yl) methyl) benzoic acid (95 mg, 38.70% yield) as a yellow solid.

LCMS (ESI) calcd for C₄₁H₅₄N₄O₉ [M+H]⁺ m/z=747.40, found 747.45.

### Step 3: tert-butyl (tert-butoxycarbonyl)(7-(3-((2-((tert-butoxycarbonyl)amino)ethyl)carbamoyl)benzyl)-1-(2-((tert-butoxycarbonyl)oxy)-2-methylpropyl)-2-butyl-1H-imidazo[4,5-c]quinolin-4-yl)carbamate

To a solution of 3-((4-(bis(tert-butoxycarbonyl)amino)-1-(2-((tert-butoxycarbonyl)oxy)-2-methylpropyl)-2-butyl-1H-imidazo[4,5-c]quinolin-7-yl)methyl)benzoic acid (95 mg, 0.13 mmol), tert-butyl N-(2-aminoethyl)carbamate (24.36 mg, 0.15 mmol) and N,N-Diisopropylethylamine (49.12 mg, 0.38 mmol) in DMF (8 mL) was added a solution of 50% T₃P (60.47 mg, 0.19 mmol) in EtOAc dropwise at RT. The reaction mixture was stirred at RT for 3 hours. The mixture was quenched with water (60 mL) and extracted with EtOAc (40 mL×3). The combined organic layers were washed with brine, dried over Na₂SO₄ and concentrated under vacuum. The residue was purified by flash column chromatography on silica gel (PE/EtOAc=10:1) to afford tert-butyl (tert-butoxycarbonyl)(7-(3-((2-((tert-butoxycarbonyl)amino)ethyl)carbamoyl)benzyl)-1-(2-((tert butoxycarbonyl)oxy)-2-methylpropyl)-2-butyl-1H-imidazo[4,5-c]quinolin-4-yl)carbamate (110 mg, 95.18% yield) as a yellow solid.

LCMS (ESI) calcd for C₄₈H₆₈N₆O₁₀ [M+H]⁺ ms/z=889.51, found 889.45.

### Step 4: 3-((4-amino-2-butyl-1-(2-hydroxy-2-methylpropyl)-1H-imidazo[4,5-c]quinolin-7-yl)methyl)-N-(2-aminoethyl)benzamide (Compound 64)

To a solution of tert-butyl (tert-butoxycarbonyl)(7-(3-((2-((tert-butoxycarbonyl)amino)ethyl)carbamoyl)benzyl)-1-(2-((tert butoxycarbonyl)oxy)-2-methylpropyl)-2-butyl-1H-imidazo[4,5-c]quinolin-4-yl)carbamate (110 mg, 0.16 mmol) in DCM (4 mL) was added TFA (1 mL) at RT. The reaction was stirred at RT for 3 hours. The resulting mixture was evaporated to give the TM. The residue was purified by Prep-HPLC (Gemini 5 um C18 column, 150*21.2 mm, eluting with 15% to 60% MeCN/H₂O containing 0.1% FA) to afford 3-((4-amino-2-butyl-1-(2-hydroxy-2-methylpropyl)-1H-imidazo[4,5-c]quinolin-7-yl)methyl)-N-(2-aminoethyl)benzamide (50 mg, 83.01% yield) as a white solid.

LCMS (ESI) calcd for C₂₈H₃₆N₆O₂ [M+H]⁺ ms/z=489.25, found 489.30 ¹H NMR (400 MHz, DMSO-d₆, ppm) δ 13.82 (s, 1H), 8.89 (s, 2H), 8.65 (t, J=5.5 Hz, 1H), 8.43 (d, J=8.6 Hz, 1H), 7.87 (s, 2H), 7.80 (s, 1H), 7.74 (d, J=7.3 Hz, 1H), 7.57 (s, 1H), 7.46 (dt, J=15.0, 7.5 Hz, 2H), 7.35 (d, J=8.6 Hz, 1H), 4.82 (s, 1H), 4.43 (s, 2H), 4.19 (s, 2H), 3.49 (q, J=6.0 Hz, 2H), 3.04 (t, J=7.7 Hz, 2H), 2.98 (s, 2H), 1.87-1.74 (m, 2H), 1.51-1.34 (m, 2H), 1.18 (s, 6H), 0.94 (t, J=7.3 Hz, 3H).

### Example 37

### Synthesis of Compound 65

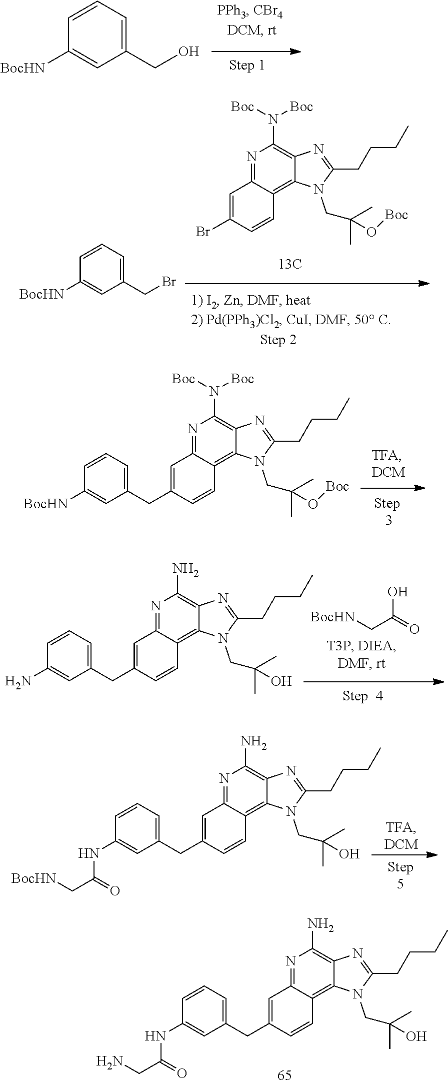

### Step 1: tert-butyl (3-(bromomethyl)phenyl)carbamate

To a solution of tert-butyl N-[3-(hydroxymethyl)phenyl]carbamate (1 g, 4.5 mmol), Triphenylphosphine (1.77 g, 6.75 mmol) in DCM (40 mL) was added carbon tetrabromide (2.24 g, 6.75 mmol) at 0° C. The reaction mixture was stirred at RT for 2 hours. The mixture was quenched with water (50 mL) and extracted with DCM (50 mL×3). The combined organic layers were washed with brine, dried over Na₂SO₄, concentrated under vacuum to give the crude. The residue was purified by flash column chromatography on silica gel (PE/EtOAc=10:1) to afford tert-butyl (3-(bromomethyl)phenyl)carbamate (1 g, 76.54% yield) as a white solid.

¹H NMR (400 MHz, DMSO-d₆, ppm) δ 9.43 (s, 1H), 7.63 (s, 1H), 7.30 (d, J=8.6 Hz, 1H), 7.22 (t, J=7.8 Hz, 1H), 7.03 (d, J=7.4 Hz, 1H), 4.64 (s, 2H), 1.48 (s, 9H).

### Step 2: tert-butyl (tert-butoxycarbonyl)(7-(3-((tert-butoxycarbonyl)amino)benzyl)-1-(2-((tert-butoxycarbonyl)oxy)-2-methylpropyl)-2-butyl-1H-imidazo[4,5-c]quinolin-4-yl)carbamate

A mixture of Zn power (790 mg, 12.18 mmol) and I₂ (91.34 mg, 0.36 mmol) in a 100 mL three-necked flask was evacuated and backfilled with N₂ three times and then charged with N₂. The flask was heated carefully with a hot air blower until all I₂ was sublimated. Then a solution of tert-butyl (3-(bromomethyl)phenyl) carbamate (500 mg, 1.74 mmol) in DMF (6 mL) was added intermediately via a syringe. The mixture was stirred for 10 minutes while keeping the temperature was about 70° C. Then the heater and stirrer were removed. The upper layer clear solution was added to a stirred solution of tert-butyl (7-bromo-1-(2-((tert-butoxycarbonyl)oxy)-2-methylpropyl)-2-butyl-1H-imidazo[4,5-c]quinolin-4-yl)(tert-butoxycarbonyl) carbamate (200 mg, 0.26 mmol), Copper(I) iodide (11 mg, 0.06 mmol) and Pd (PPh₃)₂Cl₂ (40.42 mg, 0.57 mmol) in DMF (3 mL) under an atmosphere of N₂. The mixture was heated at 50° C. for 1 hour. Then the mixture was quenched with water (50 mL) and extracted with EtOAc (50 mL×3). The combined organic layers were washed with brine, dried over Na₂SO₄, concentrated under vacuum. The residue was purified by flash column chromatography on silica gel (PE/EtOAc=2:1) to afford tert-butyl (tert-butoxycarbonyl)(7-(3-((tert-butoxycarbonyl)amino)benzyl)-1-(2-((tert-butoxycarbonyl)oxy)-2-methylpropyl)-2-butyl-1H-imidazo[4,5-c]quinolin-4-yl)carbamate (160 mg, 67.67% yield) as a yellow solid.

LCMS (ESI) calcd for C₄₅H₆₃N₅O₉ [M+H]⁺ ms/z=818.47, found 818.45.

### Step 3: 1-(4-amino-7-(3-aminobenzyl)-2-butyl-1H-imidazo[4,5-c]quinolin-1-yl)-2-methylpropan-2-ol

A solution of tert-butyl (tert-butoxycarbonyl)(7-(3-((tert-butoxycarbonyl)amino)benzyl)-1-(2-((tert-butoxycarbonyl)oxy)-2-methylpropyl)-2-butyl-1H-imidazo[4,5-c]quinolin-4-yl)carbamate (160 mg, 0.19 mmol) in DCM:TFA=2:1 (6 mL) was stirred for 3 hours. Then the mixture was adjusted to pH=8-9 with NaHCO₃ saturated solution and extracted with DCM (50 mL×3). The combined organic layers were washed with brine, dried over Na₂SO₄, filtered and concentrated to afford 1-(4-amino-7-(3-aminobenzyl)-2-butyl-1H-imidazo[4,5-c]quinolin-1-yl)-2-methylpropan-2-ol (80 mg, 98.28% yield) as a yellow solid.

LCMS (ESI) calcd for C₂₅H₃₁N₅O [M+H]⁺ ms/z=418.26, found 418.20.

### Step 4: tert-butyl (2-((3-((4-amino-2-butyl-1-(2-hydroxy-2-methylpropyl)-1H-imidazo[4,5-c]quinolin-7-yl)methyl)phenyl)amino)-2-oxoethyl)carbamate

To a solution of 1-{4-amino-7-[(3-aminophenyl)methyl]-2-butylimidazo[4,5-c]quinolin-1-yl}-2-methylpropan-2-ol (80 mg, 0.19 mmol), (tert-butoxycarbonyl)glycine (30.21 mg, 0.17 mmol) and DIEA (49.52 mg, 0.38 mmol) in DMF (8 mL) was added a solution of 50% T₃P (91.44 mg, 0.29 mmol) in EtOAc dropwise at RT. The reaction mixture was stirred at RT for 3 hours. The mixture was quenched with water (50 mL) and extracted with EtOAc (40 mL×3). The combined organic layers were washed with brine, dried over Na₂SO₄, filtered and concentrated to give the crude. The residue was purified by silica gel chromatography (PE/EtOAc=10:1) to afford tert-butyl (2-((3-((4-amino-2-butyl-1-(2-hydroxy-2-methylpropyl)-1H-imidazo[4,5-c]quinolin-7-yl)methyl)phenyl)amino)-2-oxoethyl)carbamate (110 mg, 95.96% yield) as a yellow oil.

LCMS (ESI) calcd for C₃₂H₄₂N₆O₄ [M+H]⁺ ms/z=575.33, found 575.20.

### Step 5: 2-amino-N-(3-((4-amino-2-butyl-1-(2-hydroxy-2-methylpropyl)-1H-imidazo[4,5-c]quinolin-7-yl)methyl)phenyl)acetamide (Compound 65)

To a solution of tert-butyl (2-((3-((4-amino-2-butyl-1-(2-hydroxy-2-methylpropyl)-1H-imidazo[4,5-c]quinolin-7-yl)methyl)phenyl)amino)-2-oxoethyl)carbamate (110 mg, 0.19 mmol) in DCM (4 mL) was added TFA (2 mL) at RT. The mixture was stirred at RT for 3 hours. The resulting mixture was concentrated and purified by Prep-HPLC (Gemini 5 um C18 column, 150*21.2 mm, eluting with 20% to 85% MeCN/H₂O containing 0.1% FA) to afford 2-amino-N-(3-((4-amino-2-butyl-1-(2-hydroxy-2-methylpropyl)-1H-imidazo[4,5-c]quinolin-7-yl)methyl)phenyl)acetamide (37 mg, 40.33% yield) as a white solid.

LCMS (ESI) calcd for C₂₇H₃₄N₆O₂ [M+H]⁺ ms/z=475.28, found 475.20.

¹H-NMR (400 MHz, DMSO-d₆) 13.42 (s, 1H), 10.37 (s, 1H), 8.75 (s, 1H), 8.43 (d, J=8.6 Hz, 1H), 8.06 (s, 3H), 7.55 (s, 1H), 7.48 (d, J=8.3 Hz, 1H), 7.44 (s, 1H), 7.35-7.31 (m, 2H), 7.06 (d, J=7.6 Hz, 1H), 4.81 (s, 1H), 4.54 (s, 2H), 4.12 (s, 2H), 3.74 (d, J=5.2 Hz, 2H), 3.04 (t, J=7.5 Hz, 2H), 1.82-1.79 (m, 2H), 1.43 (dd, J=14.9, 7.4 Hz, 2H), 1.23-1.18 (m, 6H), 0.94 (t, J=7.3 Hz, 3H).

### Example 38

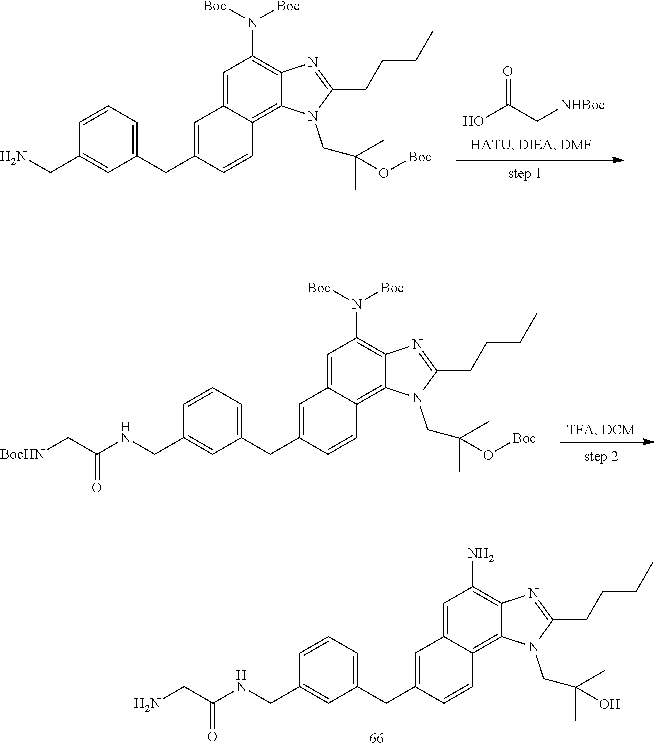 [SMILES: CCCCc1nc2c(N(C(=O)OC(C)(C)C)C(=O)OC(C)(C)C)cc3cc(Cc4cccc(CN)c4)ccc3c2n1CC(C)(C)OC(=O)OC(C)(C)C ; CC(C)(C)OC(=O)NCC(=O)O ; CCCCc1nc2c(N(C(=O)OC(C)(C)C)C(=O)OC(C)(C)C)cc3cc(Cc4cccc(CNC(=O)CNC(=O)OC(C)(C)C)c4)ccc3c2n1CC(C)(C)OC(=O)OC(C)(C)C ; CCCCc1nc2c(N)cc3cc(Cc4cccc(CNC(=O)CN)c4)ccc3c2n1CC(C)(C)O]

### Step 1: Preparation of tert-butyl (tert-butoxycarbonyl)(7-(3-((2-((tert-butoxycarbonyl)amino)acetamido) methyl)benzyl)-1-(2-((tert-butoxycarbonyl)oxy)-2-methylpropyl)-2-butyl-1H-imidazo[4,5-c]quinolin-4-yl)carbamate

To a solution of tert-butyl (7-(3-(aminomethyl)benzyl)-1-(2-((tert-butoxycarbonyl)oxy)-2-methylpropyl)-2-butyl-1H-imidazo[4,5-c]quinolin-4-yl)(tert-butoxycarbonyl)carbamate (220 mg, 0.30 mmol) and HATU (171 mg, 0.45 mmol) in DMF (10 mL) was added (tert-butoxycarbonyl)glycine (53 mg, 0.30 mmol) and DIEA (58 mg, 0.45 mmol). The reaction mixture was stirred at RT for 16 hours. The mixture was quenched with water (80 mL) and extracted with EtOAc (40 mL×3). The combined organic layers were washed with brine, dried over Na₂SO₄, filtered, concentrated to give the crude. The residue was purified by silica gel chromatography (PE/EtOAc=10/1) to afford tert-butyl (tert-butoxycarbonyl)(7-(3-((2-((tert-butoxycarbonyl)amino) acetamido) methyl)benzyl)-1-(2-((tert-butoxycarbonyl)oxy)-2-methylpropyl)-2-butyl-1H-imidazo[4,5-c]quinolin-4-yl) carbamate (120 mg, 44.9% yield) as a yellow oil.

LCMS (ESI) calcd for C₄₈H₆₉N₆O₁₀ [M+H]⁺ m/z=889.51, found 889.40.

### Step 2. Preparation of 2-amino-N-(3-((4-amino-2-butyl-1-(2-hydroxy-2-methylpropyl)-1H-imidazo[4,5-c]quinolin-7-yl)methyl)benzyl)acetamide

To a solution of tert-butyl (tert-butoxycarbonyl)(7-(3-((2-((tert-butoxycarbonyl)amino) acetamido)methyl) benzyl)-1-(2-((tert-butoxycarbonyl)oxy)-2-methylpropyl)-2-butyl-1H-imidazo[4,5-c]quinolin-4-yl)carbamate (120 mg, 0.13 mmol) in DCM (1.5 mL) was added TFA (0.5 mL) at room temperature. The reaction mixture was stirred at room temperature for 2 hours. After the reaction was completed, the mixture was concentrated. The residue was dissolved in water and adjusted to pH=8-9 with saturated aqueous NaHCO₃. The resulting solution was extracted with DCM (10 mL×3). The combined organic phases were washed with brine, dried over sodium sulfate, concentrated under vacuum. The residue was purified by prep-HPLC (Gemini 5 um C18 column, 150*21.2 mm, eluting with 25% to 80% MeCN/H₂O containing 0.1% NH₃) to 2-amino-N-(3-((4-amino-2-butyl-1-(2-hydroxy-2-methylpropyl)-1H-imidazo[4,5-c]quinolin-7-yl)methyl)benzyl)acetamide (Compound 66) (20 mg, 31.4% yield) as a white solid.

¹H NMR (400 MHz, MeOD, ppm) δ 8.43 (d, J=8.6 Hz, 1H), 7.54 (s, 1H), 7.43 (d, J=8.6 Hz, 1H), 7.31 (dd, J=16.5, 9.0 Hz, 2H), 7.21 (d, J=7.6 Hz, 2H), 4.86 (s, 2H), 4.44 (s, 2H), 4.17 (s, 2H), 3.72 (s, 2H), 3.20-3.07 (m, 2H), 2.02-1.87 (m, 2H), 1.63-1.46 (m, 2H), 1.31 (s, 6H), 1.04 (t, J=7.4 Hz, 3H).

LCMS (ESI) calcd. for C₂₈H₃₇N₆O₂ [M+H]⁺ m/z 489.30, found 489.25.

### Example 39

### Synthesis of Compound 72

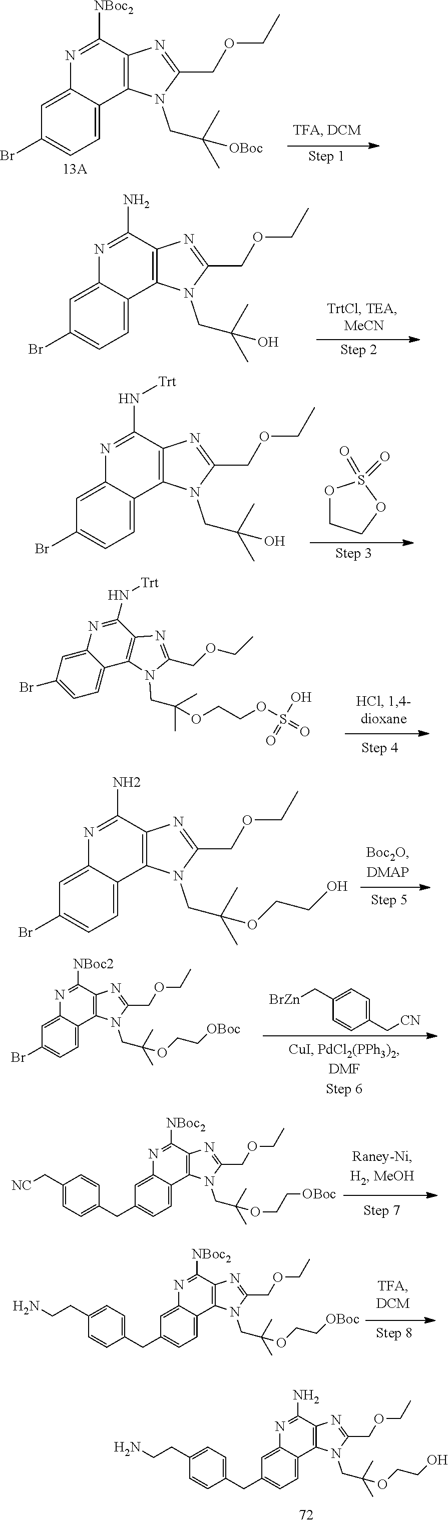

### Step 1: Preparation of 1-(4-amino-7-bromo-2-(ethoxymethyl)-1H-imidazo[4,5-c]quinolin-1-yl)-2-methylpropan-2-ol

To a solution of di-tert-butyl (7-bromo-1-(2-((tert-butoxycarbonyl)oxy)-2-methylpropyl)-2-(ethoxymethyl)-1H-imidazo[4,5-c]quinolin-4-yl)iminodicarbonate (6 g, 8.65 mmol) in DCM (30 mL) was added TFA (30 mL) dropwise at 0° C. The mixture was stirred at RT for 2 hours. The resulting mixture was concentrated in vacuo. The residue was adjusted to pH=8 with saturated sodium bicarbonate in aqueous solution and extracted with EA (80 mL×3). The organic phases were washed with brine, dried over Na₂SO₄, concentrated to give the crude title compound.

LCMS (ESI) calcd for C₁₇H₂₁BrN₄O₂[M+H]⁺ m/z=393.1, found 393.

### Step 2: Preparation of 1-(7-bromo-2-(ethoxymethyl)-4-(tritylamino)-1H-imidazo[4,5-c]quinolin-1-yl)-2-methylpropan-2-ol

A mixture of 1-(4-amino-7-bromo-2-(ethoxymethyl)-1H-imidazo[4,5-c]quinolin-1-yl)-2-methylpropan-2-ol (3.6 g, 9.15 mmol), TrtCl (3.8 g, 13.73 mmol) and Et₃N (2.3 g, 22.89 mmol) in MeCN (30 mL) was heated at 100′C in a microwave reactor for 0.5 h under an atmosphere of N₂. LCMS showed the reaction was completed. The resulting mixture was cooled to 0° C. with ice water. The precipitate was collected by filtration to afford 1-(7-bromo-2-(ethoxymethyl)-4-(tritylamino)-1H-imidazo[4,5-c]quinolin-1-yl)-2-methylpropan-2-ol (600 mg, 10.31% yield) as white solid.

LCMS (ESI) calcd for C₃₆H₃₅BrN₄O₂ [M+H]⁺ m/z=635.2, found 635.

### Step 3: Preparation of 2-((1-(7-bromo-2-(ethoxymethyl)-4-(tritylamino)-1H-imidazo[4,5-c]quinolin-1-yl)-2-methylpropan-2-yl)oxy)ethyl hydrogen sulfate

To a solution of NaH (190 mg, 60%) in DMF (5 mL) was added a solution of 1-(7-bromo-2-(ethoxymethyl)-4-(tritylamino)-1H-imidazo[4,5-c]quinolin-1-yl)-2-methylpropan-2-ol (600 mg, 0.95 mmol) in DMF (8 mL) dropwise. The mixture was stirred at RT for 1 h. Then 1,3,2-dioxathiolane 2,2-dioxide (350 mg, 2.84 mmol) was added to the mixture. The mixture was stirred at RT for 2 hours. LCMS showed the reaction was completed. The resulting mixture was quenched with water (30 mL) and extracted with EA (30 mL×3). The combine organic phases were washed with brine, dried over Na₂SO₄, concentrated to give the crude title compound. The residue was purified by combi-flash (PE:EA 1:2) to afford 2-((1-(7-bromo-2-(ethoxymethyl)-4-(tritylamino)-1H-imidazo[4,5-c]quinolin-1-yl)-2-methylpropan-2-yl)oxy)ethyl hydrogen sulfate (500 mg, 69.74% yield) as yellow solid.

LCMS (ESI) calcd for C₃₈H₃₉BrN₄O₆S [M+H]⁺ m/z=759.2, found 759.

### Step 4: Preparation of 2-((1-(4-amino-7-bromo-2-(ethoxymethyl)-1H-imidazo[4,5-c]quinolin-1-yl)-2-methylpropan-2-yl)oxy)ethan-1-ol

To a solution of 2-((1-(7-bromo-2-(ethoxymethyl)-4-(tritylamino)-1H-imidazo[4,5-c]quinolin-1-yl)-2-methylpropan-2-yl)oxy)ethyl hydrogen sulfate (500 mg, 0.66 mmol) in 1,4-dioxane (3 mL) was added HCl-1,4-dioxane (3 mL) dropwise at 0° C. The mixture was stirred at RT for 1 h. The resulting mixture was concentrated in vacuo. The residue was adjusted to pH=8 with saturated sodium bicarbonate in aqueous solution. The resulting mixture was quenched with water (30 mL) and extracted with EA (30 mL×3). The combine organic phases were washed with brine, dried over Na₂SO₄, concentrated to give the crude 2-((1-(4-amino-7-bromo-2-(ethoxymethyl)-1H-imidazo[4,5-c]quinolin-1-yl)-2-methylpropan-2-yl) oxy) ethan-1-ol (300 mg).

LCMS (ESI) calcd for C₁₉H₂₅BrN₄O₃[M+H]⁺ m/z=437.1, found 437.

### Step 5: Preparation of di-tert-butyl (7-bromo-1-(2-(2-((tert-butoxycarbonyl)oxy)ethoxy)-2-methylpropyl)-2-(ethoxymethyl)-1H-imidazo[4,5-c]quinolin-4-yl)iminodicarbonate

To a solution of 2-((1-(4-amino-7-bromo-2-(ethoxymethyl)-1H-imidazo[4,5-c]quinolin-1-yl)-2-methylpropan-2-yl)oxy)ethan-1-ol (300 mg, 0.69 mmol) and DMAP (33 mg 0.28 mmol) in MeCN (5 mL) was added Boc₂O (1050 mg, 4.80 mmol) at 75° C. The mixture was stirred at 75° C. for 4 hours. The resulting mixture was diluted with water (30 mL) and extracted with EA (30 mL×3). The combine organic phases were washed with brine, dried over Na₂SO₄, concentrated to give the crude title compound. The residue was purified by combi-flash (PE:EA 2:1) to afford di-tert-butyl (7-bromo-1-(2-(2-((tert-butoxycarbonyl)oxy)ethoxy)-2-methylpropyl)-2-(ethoxymethyl)-1H-imidazo[4,5-c]quinolin-4-yl)iminodicarbonate (100 mg, 19.76% yield) as yellow solid.

LCMS (ESI) calcd for C₃₄H₄₉BrN₄O₉[M+H]⁺ m/z=736.3, found 736.

### Step 6: Preparation of di-tert-butyl (1-(2-(2-((tert-butoxycarbonyl)oxy)ethoxy)-2-methylpropyl)-7-(4-(cyanomethyl)benzyl)-2-(ethoxymethyl)-1H-imidazo[4,5-c]quinolin-4-yl)iminodicarbonate

To a solution of di-tert-butyl (7-bromo-1-(2-(2-((tert-butoxycarbonyl)oxy)ethoxy)-2-methylpropyl)-2-(ethoxymethyl)-1H-imidazo[4,5-c]quinolin-4-yl)iminodicarbonate (100 mg, 0.14 mmol), Pd(PPh₃)Cl₂(20 mg, 0.03 mmol) and CuI (5 mg, 0.027 mmol) in DMF (3 mL) was added a solution of (4-(cyanomethyl)benzyl) zinc(II) bromide in DMF (5 mL). The mixture was stirred at 50′C for 1 hour. LCMS showed the reaction was completed. The resulting mixture was diluted with water (30 mL) and extracted with EA (20 mL×3). The combine organic phases were washed with brine, dried over Na₂SO₄, concentrated to give the crude title compound. The residue was purified by combi-flash (PE:EA 1:2) to afford di-tert-butyl (1-(2-(2-((tert-butoxycarbonyl) oxy)ethoxy)-2-methylpropyl)-7-(4-(cyanomethyl)benzyl)-2-(ethoxymethyl)-1H-imidazo[4,5-c]quinolin-4-yl)iminodicarbonate (80 mg, 74.77% yield) as yellow solid.

LCMS (ESI) calcd for C₄₃H₅₇N₅O₉ [M+H]⁺ m/z=788.4, found 788.

### Step 7: Preparation of di-tert-butyl (7-(4-(2-aminoethyl)benzyl)-1-(2-(2-((tert-butoxycarbonyl)oxy)ethoxy)-2-methylpropyl)-2-(ethoxymethyl)-1H-imidazo[4,5-c]quinolin-4-yl)iminodicarbonate

To a solution of di-tert-butyl (1-(2-(2-((tert-butoxycarbonyl)oxy)ethoxy)-2-methylpropyl)-7-(4-(cyanomethyl)benzyl)-2-(ethoxymethyl)-1H-imidazo[4,5-c]quinolin-4-yl)iminodicarbonate (80 mg, 0.10 mmol) in NH₃-MeOH (3 mL) was added the right amount of Raney-Ni washed with MeOH. The mixture was stirred at RT for 2 hours under H₂. LCMS showed the reaction was completed. Then the mixture was filtered through celite and concentrated under vacuo to give di-tert-butyl (7-(4-(2-aminoethyl)benzyl)-1-(2-(2-((tert-butoxycarbonyl)oxy)ethoxy)-2-methylpropyl)-2-(ethoxymethyl)-1H-imidazo[4,5-c]quinolin-4-yl)iminodicarbonate (60 mg, 74.6% yield).

LCMS (ESI) calcd for C₄₃H₆N₅O₉[M+H]⁺ m/z=792.4, found 792.

### Step 8: Preparation of 2-((1-(4-amino-7-(4-(2-aminoethyl)benzyl)-2-(ethoxymethyl)-1H-imidazo[4,5-c]quinolin-1-yl)-2-methylpropan-2-yl)oxy)ethan-1-ol (Compound 72)

To a solution of di-tert-butyl (7-(4-(2-aminoethyl)benzyl)-1-(2-(2-((tert-butoxycarbonyl)oxy)ethoxy)-2-methylpropyl)-2-(ethoxymethyl)-1H-imidazo[4,5-c]quinolin-4-yl)iminodicarbonate (60 mg, 0.08 mmol) in DCM (2 mL) was added TFA (1 mL) dropwise at 0° C. The mixture was stirred at RT for 2 hours. LCMS showed the reaction was completed. The reaction was concentrated and purified by Prep-HPLC (Column: Gemini-C18, 150×21.2 mm, 5 um; Mobile Phase: ACN-H₂O (0.1% FA), Gradient: 10-40) to give Compound 72 (6.05 mg, 16.24% yield) as white solid.

LCMS (ESI) calcd for C₂₈H₃₇N₅O₃ [M+H]⁺ m/z=492.3, found 492.

¹H NMR (400 MHz, MeOD) δ 8.37 (s, 1H), 8.20 (d, J=8.6 Hz, 1H), 7.39 (s, 1H), 7.16 (dd, J=23.4, 8.2 Hz, 5H), 5.24 (t, J=4.6 Hz, 1H), 4.02 (s, 2H), 3.52 (q, J=7.0 Hz, 2H), 3.35 (t, J=5.0 Hz, 2H), 3.30-3.24 (m, 2H), 3.11-3.00 (m, 2H), 2.92-2.76 (m, 2H), 2.13-2.04 (m, 1H), 1.96-1.87 (m, 2H), 1.14 (t, J=7.0 Hz, 11H), 0.80 (t, J=6.9 Hz, 2H).

### Example 40

### Synthesis of Compound 147

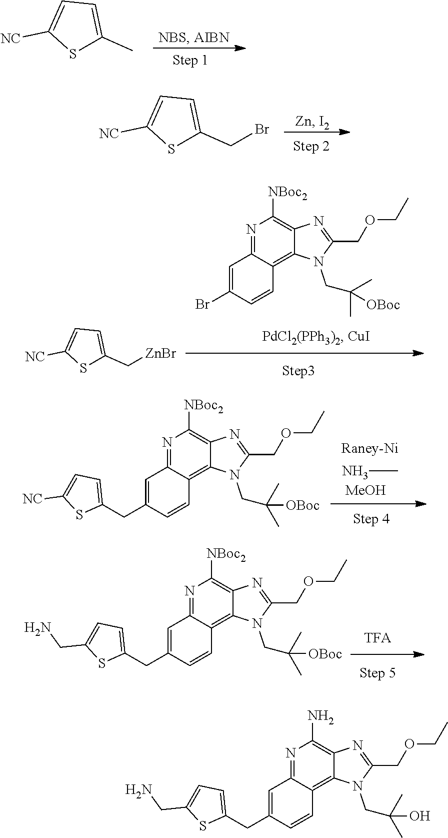

### Step 1. Preparation of 5-(bromomethyl)thiophene-2-carbonitrile

To a solution of 5-methylthiophene-2-carbonitrile (5.0 g, 40.6 mmol) in CCl₄ (100 mL) was added NBS (8.0 g, 44.9 mmol) and AIBN (0.33 g, 2.03 mmol) at room temperature. The mixture was stirred at 80° C. for 3 hours. HPLC showed the reaction was completed. The reaction was diluted with EA and filtered. The filtrated was concentrated in vacuo to give the crude. The residue was purified by Prep-HPLC (Column: Gemini-C18, 250×50 mm, 10 um; Mobile Phase: ACN-H₂O (0.1% TFA), Gradient: 10%-65%) to give the product (6.02 g, 73.4% yield) as a white solid.

LCMS (ESI) calcd for C₆H₄BrNS [M+H]⁺ m/z=201.92 203.92, found no MS.

¹H NMR (400 MHz, CDCl₃) δ 7.48 (d, J=3.8 Hz, 1H), 7.10 (d, J=3.8 Hz, 1H), 4.66 (s, 2H).

### Step 2. Preparation of ((5-cyanothiophen-2-yl)methyl)zinc(II) bromide

A mixture of Zn (1.75 g, 26.8 mmol) and 12 (0.15 g, 0.59 mmol) in N₂ protection was stirred 5 minutes under heat. Then 5-(bromomethyl)thiophene-2-carbonitrile (905 mg, 4.46 mmol) in DMF (6 mL) was added to the mixture under heat. Then the mixture was stirred for 5 minutes and used in the next step.

### Step 3. Preparation of tert-butyl(tert-butoxycarbonyl)(1-(2-((tert-butoxycarbonyl)oxy)-2-methylpropyl)-2-(ethoxymethyl)-7-((5-cyanothiophen-2-yl)methyl)-1H-imidazo[4,5-c]quinolin-4-yl)carbamate

To a solution of tert-butyl(7-bromo-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate (298 mg, 0.43 mmol), PdCl₂(PPh₃)₂(120 mg, 0.17 mmol) and CuI (100 mg, 0.53 mmol) in DMF (3 mL) was added ((5-cyanothiophen-2-yl)methyl)zinc(II) bromide (5.5 mL) under N₂. Then the mixture was stirred at 50° C. for 1 hour. TLC (n-Heptane:EA=1:1) showed the reaction was OK. It was poured into saturated NH₄Cl (aq.) (40 mL) and extracted with EA (30 mL*2), the combined organic phases were washed with water (20 mL*2) and dried over anhydrous Na₂SO₄, concentrated on vacuum to give the crude. The residue was purified by silica gel column chromatography (eluting with PE:EA=3:1) to give the product (258 mg, 86.2% yield) as a yellow oil.

LCMS (ESI) calcd for C₃₈H₄₉N₅O₈S [M+H]⁺ m/z=736.33, found 736.6.

### Step 4. Preparation of tert-butyl(7-((5-(aminomethyl)thiophen-2-yl)methyl)-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate

A solution of tert-butyl (tert-butoxycarbonyl)(1-(2-((tert-butoxycarbonyl)oxy)-2-methylpropyl)-2-(ethoxymethyl)-7-((5-cyanothiophen-2-yl)methyl)-1H-imidazo[4,5-c]quinolin-4-yl)carbamate (258 mg, 0.44 mmol) and Raney-Ni (about 1 g) in NH₃-MeOH (7M) (20 mL) was stirred under hydrogen (1 atm) at room temperature. The reaction mixture was stirred at room temperature for 2 hours. HPLC showed the reaction was OK. The mixture was filtered through Celite and the filtrate was concentrated to give the crude product (317 mg, 100% yield).

LCMS (ESI) calcd for C₃₈H₅₃N₅O₈S [M+H]⁺ m/z=740.36, found 740.7.

### Step 5. Preparation of 1-(4-amino-7-((5-(aminomethyl)thiophen-2-yl)methyl)-2-(ethoxymethyl)-1H-imidazo[4,5-c]quinolin-1-yl)-2-methylpropan-2-ol

To a solution of Tert butyl (7-((5-(aminomethyl)thiophen-2-yl)methyl)-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate (317 mg, 0.41 mmol) in DCM (2 mL) was added TFA (1 mL) drop wise at room temperature. The reaction mixture was stirred at room temperature for 3 hours. HPLC showed the reaction was OK. The solution was removed under reduced pressure to afford the crude product. The residue was purified by Prep-HPLC (column: Gemini-C18 250×21.2 mm, 10 um; mobile phase: ACN-H₂O (0.1% TFA); gradient: 20%-65%) to afford Compound 147 (75 mg, 23.6% yield) as white solid.

MS (ESI) calcd for C₂₃H₂₉N₅O₂S [M+H]⁺ m/z=440.20, found 440.5.

¹H NMR (400 MHz, DMSO-d6) δ 13.93 (s, 1H), 9.14 (s, 1H), 8.46 (d, J=8.6 Hz, 1H), 8.19 (s, 3H), 7.63 (s, 1H), 7.41 (d, J=8.6 Hz, 1H), 7.07 (d, J=3.5 Hz, 1H), 6.94 (d, J=3.5 Hz, 1H), 4.95 (s, 2H), 4.71 (s, 2H), 4.34 (s, 2H), 4.16 (d, J=4.7 Hz, 2H), 3.54 (m, 2H), 1.15 (m, 9H).

### Example 41

### Synthesis of Compound 148

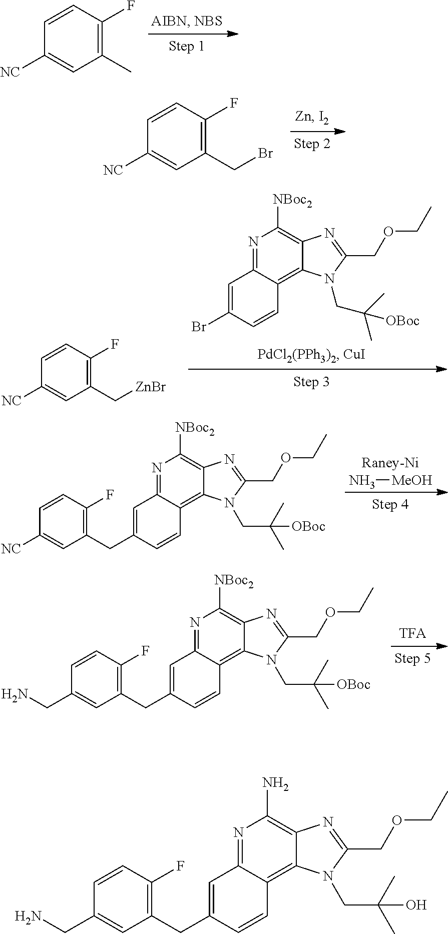

### Step 1. Preparation of 3-(bromomethyl)-4-fluorobenzonitrile

To a solution of 4-fluoro-3-methylbenzonitrile (5.0 g, 34.0 mmol) in CCl₄ (100 mL) was added NBS (6.66 g, 37.4 mmol) and AIBN (0.28 g, 1.70 mmol) at room temperature. The mixture was stirred at 80° C. for 3 hours. HPLC showed the reaction was completed. The reaction was diluted with EA and filtered. The filtrated was concentrated in vacuo to give the crude. The residue was purified by silica gel column chromatography (eluting with PE:EA=80:1) to give the product (4.02 g, 50.8% yield) as a white solid.

¹H NMR (400 MHz, CDCl₃) δ 7.75 (dd, J=6.8, 2.0 Hz, 1H), 7.63 (ddd, J=8.4, 4.7, 2.1 Hz, 1H), 7.20 (t, J=8.9 Hz, 1H), 4.48 (s, 2H).

### Step 2. Preparation of (5-cyano-2-fluorobenzyl)zinc(II) bromide

A mixture of Zn (1.75 g, 26.8 mmol) and I₂ (0.3 g, 1.18 mmol) in N₂ protection was stirred 5 minutes under heat. Then 3-(bromomethyl)-4-fluorobenzonitrile (954 mg, 4.46 mmol) in DMF (6 mL) was added to the mixture under heated. Then the mixture was stirred at room temperature for 5 minutes and used in the next step.

### Step 3. Preparation of tert-butyl (tert-butoxycarbonyl)(1-(2-((tert-butoxycarbonyl)oxy)-2-methylpropyl)-2-(ethoxymethyl)-7-(5-cyano-2-fluorobenzyl)-1H-imidazo[4,5-c]quinolin-4-yl)carbamate

To a solution tert-butyl (7-bromo-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate (200 mg, 0.43 mmol), PdCl₂(PPh₃)₂(120 mg, 0.17 mmol) and CuI (100 mg, 0.52 mmol) in DMF (3 mL) was added (3-cyano-4-fluorobenzyl)zinc(II) bromide (4 mL) under N₂. Then the mixture was stirred at 50° C. for 1 hour. HPLC and LCMS showed the reaction was OK. It was poured into water and extracted with EA (50 mL*3), the combined organic phases were washed with water and dried over Na₂SO₄, concentrated to give the crude. The residue was purified by Silica gel Column (PE:EA=3:1) to give the crude product (160 mg, 61.8% yield) as a yellow solid.

LCMS (ESI) calcd for C₄₀H₅₀FN₅O₈ [M+H]⁺ m/z=748.36, found 748.7.

### Step 4. Preparation of tert-butyl (7-(5-(aminomethyl)-2-fluorobenzyl)-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate

A solution of tert-butyl (tert-butoxycarbonyl)(1-(2-((tert-butoxycarbonyl)oxy)-2-methylpropyl)-2-(ethoxymethyl)-7-(5-cyano-2-fluorobenzyl)-1H-imidazo[4,5-c]quinolin-4-yl)carbamate (160 mg, 0.21 mmol) and Raney-Ni (about 100 mg) in NH₃-MeOH (7M) (20 mL) was stirred under hydrogen for 18 hours at room temperature. The mixture was filtered through celite and the filtrate was concentrated to give the crude product (150 mg).

LCMS (ESI) calcd for C₄₀H₅₄FN₅O₈ [M+H]⁺ m/z=752.40, found 752.7.

### Step 5. Preparation of 1-(4-amino-7-(5-(aminomethyl)-2-fluorobenzyl)-2-(ethoxymethyl)-1H-imidazo[4,5-c]quinolin-1-yl)-2-methylpropan-2-ol

To a solution of tert-butyl (7-(5-(aminomethyl)-2-fluorobenzyl)-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate (150 mg, 0.19 mmol) in DCM (5 mL) was added TFA (2 mL) dropwise at room temperature. The reaction mixture was stirred at room temperature for 18 hours. The solution was removed under reduced pressure to afford the crude product. The residue was purified by Prep-HPLC (Column: Gemini-C18, 250×21.2 mm, 10 um; Mobile Phase: ACN-H₂O (0.1% TFA), Gradient: 20%-65%) to afford Compound 148 (39.2 mg, 43.3% yield) as white solid.

MS (ESI) calcd for C₂₅H₃₀FN₅O₂[M+H]⁺ m/z=452.24, found 452.5.

### Example 42

### Synthesis of Compound 149

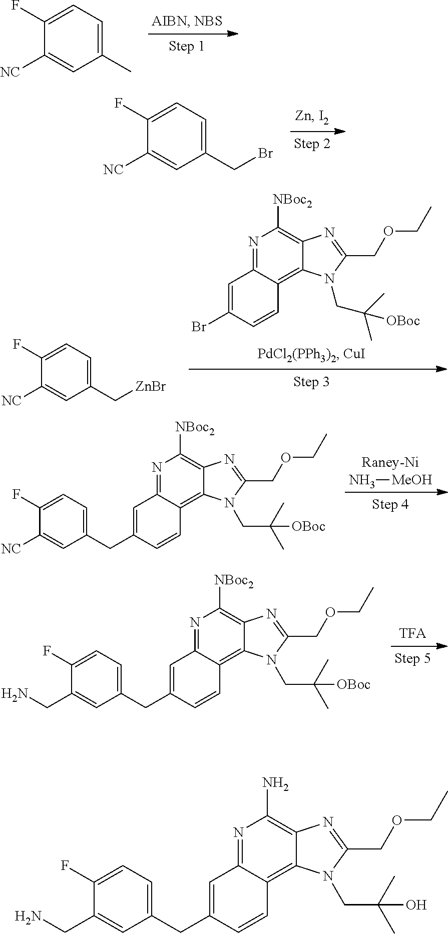

Synthetic procedures for Compound 149 were similar with those of Compound 1.

LCMS (ESI) calcd for C₅₃H₆₉N₁₁O₉S [M+H]⁺ m/z=1036.5, found 1036.4.

### Example 43

### Synthesis of Compound 150

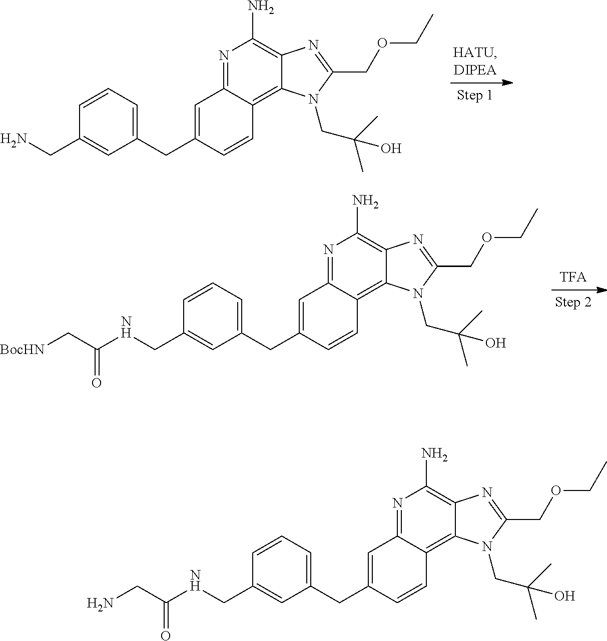

### Step 1. Preparation of tert-butyl (2-((3-((4-amino-2-(ethoxymethyl)-1-(2-hydroxy-2-methylpropyl)-1H-imidazo[4,5-c]quinolin-7-yl)methyl)benzyl)amino)-2-oxoethyl)carbamate

A solution of 1-(4-amino-7-(3-(aminomethyl)benzyl)-2-(ethoxymethyl)-1H-imidazo[4,5-c]quinolin-1-yl)-2-methylpropan-2-ol (100 mg, 0.23 mmol), Boc-glycine (16 mg, 0.09 mmol), HATU (129 mg, 0.34 mmol) and DIEA (89 mg, 0.69 mmol) in DMF (2 mL) was stirred for 2 hours at room temperature. HPLC and LC-MS showed the reaction was OK. The residue was purified by Prep-HPLC (Column: Gemini-C18, 250×21.2 mm, 10 um; Mobile Phase: ACN-H₂O (0.1% TFA), Gradient: 20%-65%) to afford product (50 mg, 37.0% yield) as white solid.

LCMS (ESI) calcd for C₃₂H₄₂N₆O₅ [M+H]⁺ m/z=591.32, found 591.4.

### Step 2. Preparation of 2-amino-N-(3-((4-amino-2-(ethoxymethyl)-1-(2-hydroxy-2-methylpropyl)-1H-imidazo[4,5-c]quinolin-7-yl)methyl)benzyl)acetamide

To a solution of tert-butyl (2-((3-((4-amino-2-(ethoxymethyl)-1-(2-hydroxy-2-methylpropyl)-1H-imidazo[4,5-c]quinolin-7-yl)methyl)benzyl)amino)-2-oxoethyl)carbamate (50 mg, 0.084 mmol) in DCM (2 mL) was added TFA (1 mL) dropwise at room temperature. The reaction mixture was stirred at room temperature for 18 hours. HPLC and LCMS showed the reaction was OK. The solution was removed under reduced pressure to afford the crude product. The residue was purified by Prep-HPLC (Column: Gemini-C18, 250×21.2 mm, 10 um; Mobile Phase: ACN-H₂O (0.1% TFA), Gradient: 20%-65%) to afford Compound 150 (10 mg, 24.1% yield) as white solid.

LCMS (ESI) calcd for C₂₇H₃₄N₆O₃ [M+H]⁺ m/z=491.27, found 491.6.

### Example 44

### Synthesis of Compound 151

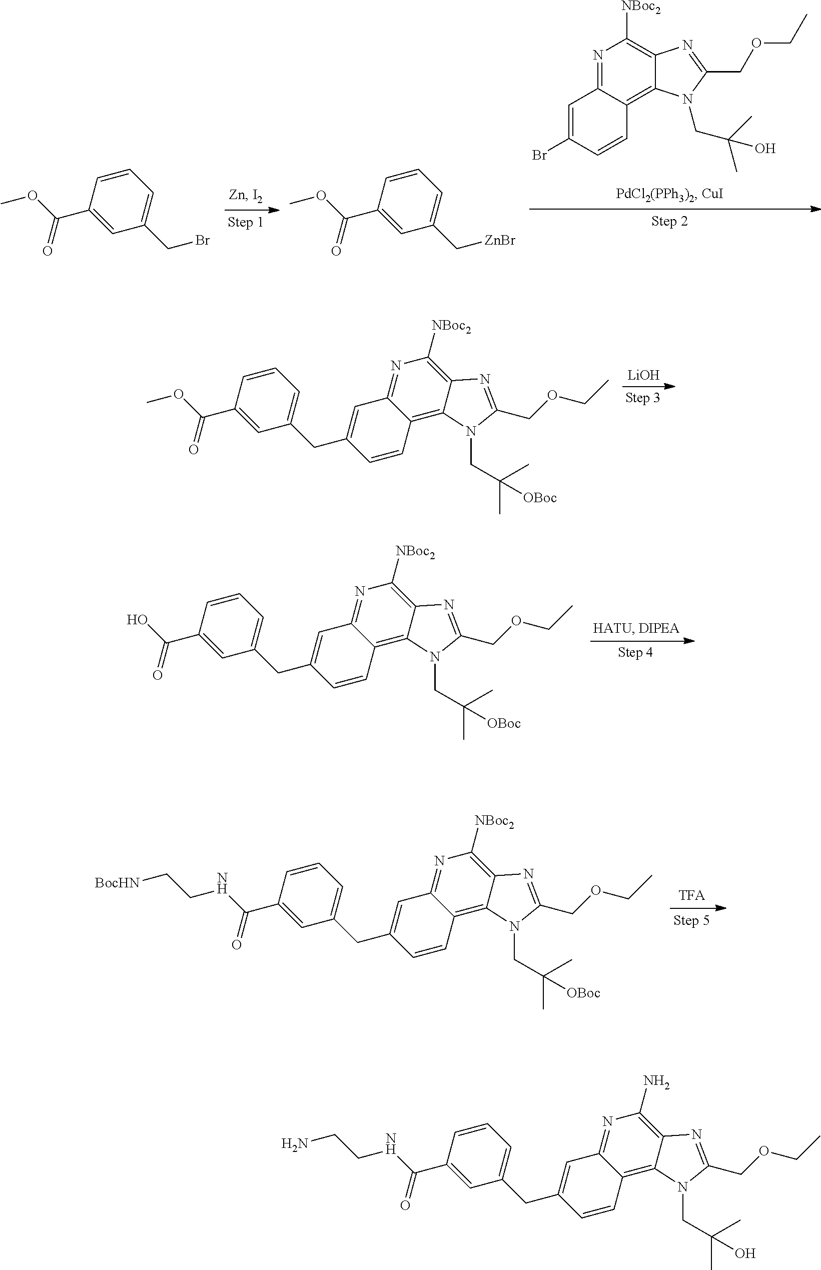

### Step 1. Preparation of (3-(methoxycarbonyl)benzyl)zinc(II) bromide

A mixture of Zn (1.75 g, 26.8 mmol) and I₂ (150 mg, 0.591 mmol) in N₂ protection was stirred 5 mins under heat. Then methyl 3-(bromomethyl)benzoate (1.02 g, 4.46 mmol) in DMF (6 mL) was added to the mixture under heat. Then the mixture was stirred for 5 mins and used in the next step.

### Step 2. Preparation of 3-((4-(bis(tert-butoxycarbonyl)amino)-1-(2-((tert-butoxycarbonyl)oxy)-2-methylpropyl)-2-(ethoxymethyl)-1H-imidazo[4,5-c]quinolin-7-yl)methyl)benzoate

To a solution of tert-butyl (7-bromo-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate (300 mg, 0.43 mmol), PdCl₂(PPh₃)₂(120 mg, 0.17 mmol) and CuI (100 mg, 0.53 mmol) in DMF (3 mL) was added ((5-cyanothiophen-2-yl)methyl)zinc(II) bromide (5.5 mL) under N₂. Then the mixture was stirred at 50° C. for 1 hour. HPLC and LCMS showed the reaction was OK. It was poured into saturated NH₄Cl (aq) (40 mL) and extracted with EA (30 mL*2), the combined organic phases were washed with water (20 mL*2) and dried over anhydrous Na₂SO₄, concentrated to give the crude. The residue was purified by silica gel column chromatography (eluting with PE:EA=3:1) to give product (310 mg, 94.2% yield) as a yellow solid.

LCMS (ESI) calcd for C₄₁H₅₄N₄O₁₀ [M+H]⁺ m/z=763.38, found 763.7.

### Step 3. Preparation of 3-((4-(bis(tert-butoxycarbonyl)amino)-1-(2-((tert-butoxycarbonyl)oxy)-2-methylpropyl)-2-(ethoxymethyl)-1H-imidazo[4,5-c]quinolin-7-yl)methyl)benzoic acid

A solution of methyl 3-((4-(bis(tert-butoxycarbonyl)amino)-1-(2-((tert-butoxycarbonyl)oxy)-2-methylpropyl)-2-(ethoxymethyl)-1H-imidazo[4,5-c]quinolin-7-yl)methyl)benzoate (310 mg, 0.43 mmol) and LiOH (62 mg, 2.58 mmol) in THF (3 mL), H₂O (3 mL) was stirred at room temperature for 16 hours. TLC showed the reaction was OK. The mixture was adjusted to pH=3 with 1M HCl (aq) and extracted with EA (10 mL*2), the combined organic phases were washed with water and dried over anhydrous Na₂SO₄, concentrated to give the crude. The residue was purified by silica gel column chromatography (eluting with PE:EA=1:1) to give the product (300 mg, 98.6% yield) as a yellow solid.

LCMS (ESI) calcd for C₄₀H₅₂N₄O₁₀ [M+H]⁺ m/z=749.37, found 749.7, 649.6.

### Step 4. Preparation of (tert-butoxycarbonyl)(7-(3-((2-((tert-butoxycarbonyl)amino)ethyl)carbamoyl)benzyl)-1-(2-((tert-butoxycarbonyl)oxy)-2-methylpropyl)-2-(ethoxymethyl)-1H-imidazo[4,5-c]quinolin-4-yl)carbamate

To a solution of 3-((4-(bis(tert-butoxycarbonyl)amino)-1-(2-((tert-butoxycarbonyl)oxy)-2-methylpropyl)-2-(ethoxymethyl)-1H-imidazo[4,5-c]quinolin-7-yl)methyl)benzoic acid (300 mg, 0.401 mmol) in DMF (12 mL) was added tert-butyl (2-aminoethyl)carbamate (70.5 mg, 0.441 mmol), HATU (228.5 mg, 0.601 mmol), DIPEA (155.2 mg, 1.203 mmol). The reaction mixture was stirred at room temperature for 1 hour. TLC showed the reaction was OK. It was poured into water and extracted with EA (60 mL*2), the combined organic phases were washed with water and dried over anhydrous Na₂SO₄, concentrated to give the crude. The residue was purified by silica gel column chromatography (eluting with PE:EA=1:1) to give the product (300 mg, 84.1% yield) as a white solid.

LCMS (ESI) calcd for C₄₇H₆₆N₆O₁₁ [M+H]⁺ m/z=891.48, found 891.7.

### Step 5. Preparation of 3-((4-amino-2-(ethoxymethyl)-1-(2-hydroxy-2-methylpropyl)-1H-imidazo[4,5-c]quinolin-7-yl)methyl)-N-(2-aminoethyl)benzamide

To a solution of tert-butyl (tert-butoxycarbonyl)(7-(3-((2-((tert-butoxycarbonyl)amino)ethyl)carbamoyl)benzyl)-1-(2-((tert-butoxycarbonyl)oxy)-2-methylpropyl)-2-(ethoxymethyl)-1H-imidazo[4,5-c]quinolin-4-yl)carbamate (300 mg, 0.337 mmol) in DCM (12 mL) was added TFA (4 mL) dropwise at room temperature. The reaction mixture was stirred at room temperature for 3 hours. HPLC and LCMS showed the reaction was OK. The solvent was removed under reduced pressure to afford the crude product (165 mg, 100% yield), which was used in the next step without further purification.

LCMS (ESI) calcd for C₂₇H₃₄N₆O₃ [M+H]⁺ m/z=491.27, found 491.5.

¹H NMR (400 MHz, DMSO-d6) δ 13.72 (s, 1H), 9.00 (s, 1H), 8.63 (t, J=5.6 Hz, 1H), 8.43 (d, J=8.6 Hz, 1H), 7.82 (d, J=11.6 Hz, 4H), 7.75 (d, J=7.4 Hz, 1H), 7.58 (s, 1H), 7.47 (dt, J=15.0, 7.6 Hz, 2H), 7.37 (d, J=8.5 Hz, 1H), 4.93 (s, 2H), 4.69 (s, 2H), 4.20 (s, 2H), 3.54 (q, J=7.0 Hz, 2H), 3.48 (dd, J=12.1, 6.2 Hz, 2H), 2.97 (d, J=5.4 Hz, 2H), 1.15 (dd, J=13.9, 6.9 Hz, 9H).

### Example 45

### Synthesis of Compound 152

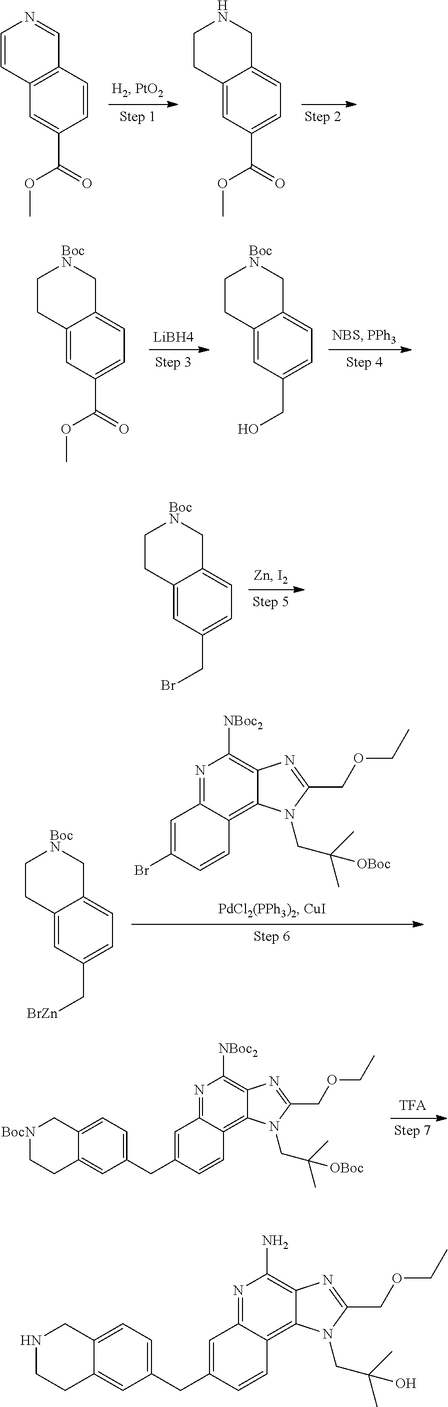

### Step 1. Preparation of methyl 1,2,3,4-tetrahydroisoquinoline-6-carboxylate

A solution of methyl isoquinoline-6-carboxylate (5.01 g, 26.76 mmol), AcOH (4.82 g, 80.30 mmol) and PtO₂ (303.9 mg, 1.338 mmol) in MeOH (200 mL) was stirred under hydrogen (1 atm) at room temperature. The reaction mixture was stirred at room temperature (18.1° C.) for 18 hours. HPLC showed the reaction was OK. The mixture was filtered through Celite and the filtrate was concentrated to give the crude product (5.51 g, 100% yield).

LCMS (ESI) calcd for C₁₁H₁₃NO₂ [M+H]⁺ m/z=192.09, found 192.4.

### Step 2. Preparation of 2-(tert-butyl) 6-methyl 3,4-dihydroisoquinoline-2,6(1H)-dicarboxylate

To a solution of methyl 1,2,3,4-tetrahydroisoquinoline-6-carboxylate (5.51 g, crude), Boc₂O (7.01 g, 32.12 mmol) in DCM (100 mL) was added Et₃N (5.5 mL, 53.52 mmol) under N₂. Then the mixture was stirred at room temperature (16.3° C.) for 1.5 hours. HPLC and LCMS showed the reaction was OK. It was washed with water (30 mL) and dried over anhydrous Na₂SO₄, concentrated to give the crude. The residue was purified by silica gel column chromatography (eluting with PE:EA=5:1 to 3:1) to give product (7.10 g, 91.3% yield) as a yellow solid.

¹H NMR (400 MHz, MeOD) δ 7.82 (d, J=6.3 Hz, 2H), 7.25 (d, J=8.5 Hz, 1H), 4.62 (s, 2H), 3.89 (s, 3H), 3.66 (t, J=5.5 Hz, 2H), 2.89 (t, J=5.9 Hz, 2H), 1.49 (s, 9H).

### Step 3. Preparation of tert-butyl 6-(hydroxymethyl)-3,4-dihydroisoquinoline-2(1H)-carboxylate

A solution of 2-(tert-butyl) 6-methyl 3,4-dihydroisoquinoline-2,6(1H)-dicarboxylate (7.10 g, 26.96 mmol) and LiBH₄ (1.77 g, 80.89 mmol) in THF (115 mL), MeOH (3 mL) was stirred at 50° C. for 4 hours. TLC showed the reaction was OK. It was poured into cold saturated NH₄Cl (aq.) (40 mL) slowly and extracted with EA (50 mL*2), the combined organic phases were washed with water (40 mL*2) and dried over anhydrous Na₂SO₄, concentrated on vacuum to give the crude. The residue was purified by silica gel column chromatography (eluting with PE:EA=3:1) to give the product (6.80 g, 100% yield) as a yellow solid.

LCMS (ESI) calcd for C₁₅H₂₁NO₃ [M+H]⁺ m/z=264.15, found 264.4.

### Step 4. Preparation of tert-butyl 6-(bromomethyl)-3,4-dihydroisoquinoline-2(1H)-carboxylate

To a solution of tert-butyl 6-(hydroxymethyl)-3,4-dihydroisoquinoline-2(1H)-carboxylate (6.31 g, 23.9787 mmol) in THF (120 mL) was added PPh₃ (18.8 g, 71.9362 mmol) at 0° C. under N₂. The mixture was stirred at 0° C. for 10 minutes. Then the reaction mixture was added NBS (17.1 g, 95.9148 mmol) in portions. The mixture was stirred at room temperature for 1 hour. TLC (n-Heptane:EA=1:1) showed the reaction was OK. It was poured into saturated H₂O (aq.) (200 mL) and extracted with EA (150 mL*3), the combined organic phases were washed with saturated NaCl (aq.) (150 mL*2) and dried over anhydrous Na₂SO₄, concentrated in vacuo to give the crude. The residue was purified by silica gel column chromatography (eluting with PE:EA=20:1) to give the product (5.97 g, 96% yield) as a white solid.

¹H NMR (400 MHz, CDCl₃) δ 7.24-7.15 (m, 2H), 7.08 (d, J=7.8 Hz, 1H), 4.56 (s, 2H), 4.46 (s, 2H), 3.64 (t, J=5.4 Hz, 2H), 2.82 (t, J=5.7 Hz, 2H), 1.49 (s, 9H).

### Step 5. Preparation of (2-(tert-butoxycarbonyl)-1,2,3,4-tetrahydroisoquinolin-6-yl)methyl)zinc(II) bromide

A mixture of Zn (1.73 g, 26.6683 mmol) and I₂ (0.25 g, 0.9778 mmol) in N₂ protection was stirred 5 minutes under heat. Then tert-butyl 6-(bromomethyl)-3,4-dihydroisoquinoline-2(1H)-carboxylate (1.45 g, 4.4447 mmol) in DMF (6 mL) was added to the mixture under heat. Then the mixture was stirred for 5 minutes and used in the next step.

### Step 6. Preparation of tert-butyl 6-((4-(bis(tert-butoxycarbonyl)amino)-1-(2-((tert-butoxycarbonyl)oxy)-2-methylpropyl)-2-(ethoxymethyl)-1H-imidazo[4,5-c]quinolin-7-yl)methyl)-3,4-dihydroisoquinoline-2(1H)-carboxylate

To a solution of tert-butyl (7-bromo-1-(2-((tert-butoxycarbonyl)oxy)-2-methylpropyl)-2-(ethoxymethyl)-1H-imidazo[4,5-c]quinolin-4-yl)(tert-butoxycarbonyl)carbama (300 mg, 0.4325 mmol), PdCl₂(PPh₃)₂(121.4 mg, 0.1730 mmol) and CuI (98.8 mg, 0.5190 mmol) in DMF (3 mL) was added ((2-(tert-butoxycarbonyl)-1,2,3,4-tetrahydroisoquinolin-6-yl)methyl)zinc(II) bromide (5.5 mL) under N₂. Then the mixture was stirred at 50° C. for 1 hour. HPLC showed the reaction was OK. It was poured into saturated NH₄Cl (aq.) (40 mL) and extracted with EA (30 mL*2), the combined organic phases were washed with water (20 mL*2) and dried over anhydrous Na₂SO₄, concentrated in vacuum to give the crude. The residue was purified by silica gel column chromatography (eluting with PE:EA=3:1) to give the product (200 mg, 98% yield) as a white solid.

MS (ESI) calcd for C₄₇H₆₅N₅O₁₀ [M+H]⁺ m/z=860.47, found 860.6.

### Step 7. Preparation of 1-(4-amino-2-(ethoxymethyl)-7-((1,2,3,4-tetrahydroisoquinolin-6-yl)methyl)-1H-imidazo[4,5-c]quinolin-1-yl)-2-methylpropan-2-ol

To a solution of tert-butyl 4-(3-((4-(bis(tert-butoxycarbonyl)amino)-1-(2-((tert-butoxycarbonyl)oxy)-2-methylpropyl)-2-(ethoxymethyl)-1H-imidazo[4,5-c]quinolin-7-yl)methyl)phenyl)piperidine-1-carboxylate (180 mg, 0.2027 mmol) in DCM (2 mL) was added TFA (1 mL) drop wise at room temperature. The reaction mixture was stirred at room temperature for 3 hours. HPLC showed the reaction was OK. The solution was removed under reduced pressure to afford the crude product. The residue was purified by Prep-HPLC (column: Gemini-C18 250×21.2 mm, 10 um; mobile phase: ACN-H₂O (0.1% TFA); gradient: 20%-65%) to afford Compound 152 (54.9 mg, 89.0% yield) as white solid.

MS (ESI) calcd for C₂₇H₃₃N₅O₂ [M+H]⁺ m/z=460.26, found 460.4.

### Example 46

### Synthesis of Compound 153

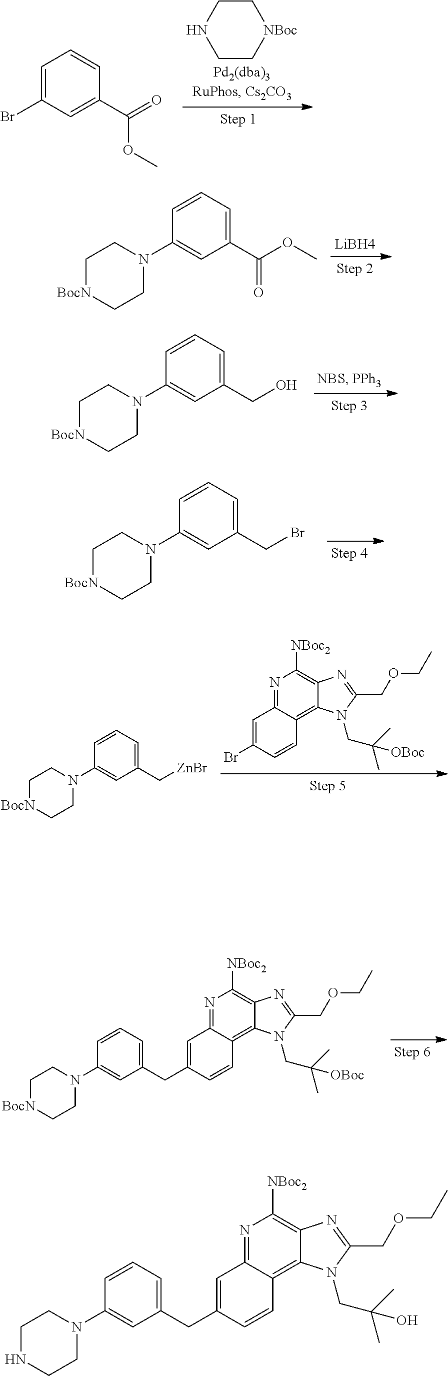

### Step 1. Preparation of tert-butyl 4-(3-(methoxycarbonyl)phenyl)piperazine-1-carboxylate

To a solution of methyl 3-bromobenzoate (5.0 g, 23.25 mmol), tert-butyl piperazine-1-carboxylate (7.58 g, 40.69 mmol), Pd₂(dba)₃ (1.06 g, 1.163 mmol) and RuPhos (1.08 g, 2.325 mmol) in DMF (100 mL) was added Cs₂CO₃ (15.2 g, 46.50 mmol) at room temperature under N₂. Then the mixture was stirred at 130° C. for 2 hours. TLC (n-Heptane:EA=3:1) showed the reaction was OK. It was poured into H₂O (150 mL) and extracted with EA (100 mL*2), the combined organic phases were washed with saturated NaCl (aq.) (100 mL*2) and dried over anhydrous Na₂SO₄, concentrated in vacuum to give the crude. The residue was purified by silica gel column chromatography (eluting with PE:EA=4:1) to give the product (7.7 g, 95% yield) as a light yellow solid.

LCMS (ESI) calcd for C₁₇H₂₄N₂O₄ [M+H]⁺ m/z=321.17, found 321.4.

### Step 2. Preparation of tert-butyl 4-(3-(hydroxymethyl)phenyl)piperazine-1-carboxylate

To a solution of tert-butyl 4-(3-(methoxycarbonyl)phenyl)piperazine-1-carboxylate (5.87 g, 18.32 mmol) in THE (120 mL) was added LiBH₄ (2.39 g, 109.9 mmol) drop wise at 0° C. under N₂. Then the mixture was stirred at 60° C. for 8 hours. TLC (n-Heptane:EA=1:1) showed the reaction was OK. It was poured into saturated NH₄Cl (aq.) (150 mL) and extracted with EA (100 mL*3), the combined organic phases were washed with saturated NaCl (aq.) (100 mL*2) and dried over anhydrous Na₂SO₄, concentrated in vacuo to give the crude. The residue was purified by silica gel column chromatography (eluting with PE:EA=2:1) to give the product (4.56 g, 94.7% yield) as a white solid.

LCMS (ESI) calcd for C₁₆H₂₄N₂O₃ [M+H]⁺ m/z=293.18, found 293.4, 237.4.

### Step 3. Preparation of tert-butyl 4-(3-(bromomethyl) phenyl) piperazine-1-carboxylate

To a solution of tert-butyl 4-(3-(hydroxymethyl) phenyl) piperazine-1-carboxylate (3.96 g, 13.54 mmol) in DCM (80 mL) was added PPh₃ (3.90 g, 14.90 mmol) and imidazole (1.10 mg, 16.25 mmol) under N₂. The mixture was added CBr₄ (4.94 g, 14.90 mmol) drop wise at 0° C. Then the mixture was stirred at 0° C. for 1 hour. TLC (n-Heptane:EA=1:1) showed the reaction was OK. The reaction mixture was diluted with H₂O (150 mL) and extracted with EA (100 mL*3), the combined organic phases were washed with saturated NaCl (aq.) (100 mL*2) and dried over anhydrous Na₂SO₄, concentrated in vacuo to give the crude. The residue was purified by silica gel column chromatography (eluting with PE:EA=3:1) to give the product (2.90 g, 60.1% yield) as a white solid.

¹H NMR (400 MHz, CDCl₃) δ 7.33-7.27 (m, 1H), 7.02-6.87 (m, 3H), 4.50 (s, 2H), 3.69-3.58 (m, 4H), 3.29-3.14 (m, 4H), 1.53 (s, 9H).

### Step 4. Preparation of (3-(4-(tert-butoxycarbonyl) piperazin-1-yl) benzyl) zinc (II) bromide

A mixture of Zn (1.74 g, 26.68 mmol) and I₂ (0.25 g, 0.9784 mmol) in N₂ protection was stirred 5 minutes under heat. Then tert-butyl 4-(3-(bromomethyl) phenyl) piperazine-1-carboxylate (1.58 g, 4.45 mmol) in DMF (6 mL) was added to the mixture under heat. Then the mixture was stirred for 5 minutes and used in the next step.

### Step 5. Preparation of tert-butyl 4-(3-((4-(bis(tert-butoxycarbonyl)amino)-1-(2-((tert-butoxycarbonyl)oxy)-2-methylpropyl)-2-(ethoxymethyl)-1H-imidazo[4,5-c]quinolin-7-yl)methyl)phenyl)piperazine-1-carboxylate

To a solution of tert-butyl (7-bromo-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate (300 mg, 0.4325 mmol), PdCl₂(PPh₃)₂(121.4 mg, 0.1730 mmol) and CuI (98.8 mg, 0.5190 mmol) in DMF (3 mL) was added (3-(4-(tert-butoxycarbonyl)piperazin-1-yl)benzyl)zinc(II) bromide (5.5 mL) under N₂. Then the mixture was stirred at 50° C. for 1 hour. HPLC showed the reaction was OK. It was poured into saturated NH₄Cl (aq.) (40 mL) and extracted with EA (30 mL*2), the combined organic phases were washed with water (20 mL*2) and dried over anhydrous Na₂SO₄, concentrated in vacuum to give the crude. The residue was purified by silica gel column chromatography (eluting with PE:EA=3:1) to give the product (120 mg, 96% yield) as a yellow solid.

LCMS (ESI) calcd for C₄₈H₆₈N₆O₁₀ [M+H]⁺ m/z=889.50, found 889.9.

### Step 7. Preparation of 1-(4-amino-2-(ethoxymethyl)-7-(3-(piperazin-1-yl)benzyl)-1H-imidazo[4,5-c]quinolin-1-yl)-2-methylpropan-2-ol

To a solution of tert-butyl 4-(3-((4-(bis(tert-butoxycarbonyl)amino)-1-(2-((tert-butoxycarbonyl)oxy)-2-methylpropyl)-2-(ethoxymethyl)-1H-imidazo[4,5-c]quinolin-7-yl)methyl)phenyl)piperazine-1-carboxylate (120 mg, 0.1349 mmol) in DCM (2 mL) was added TFA (1 mL) drop wise at room temperature. The reaction mixture was stirred at room temperature for 3 hours. HPLC showed the reaction was OK. The solution was removed under reduced pressure to afford the crude product. The residue was purified by Prep-HPLC (column: Gemini-C18 250×21.2 mm, 10 um; mobile phase: ACN-H₂O (0.1% TFA); gradient: 20%-65%) to afford Compound 153 (128 mg, 89.0% yield) as white solid.

LCMS (ESI) calcd for C₂₈H₃₆N₆O₂ [M+H]⁺ m/z=489.29, found 489.6.

¹H NMR (400 MHz, DMSO-d6) δ 13.78 (s, 1H), 8.86 (s, 3H), 8.41 (d, J=8.6 Hz, 1H), 7.57 (s, 1H), 7.37 (d, J=7.4 Hz, 1H), 7.21 (t, J=7.9 Hz, 1H), 6.97 (s, 1H), 6.86 (dd, J=8.2, 2.1 Hz, 1H), 6.77 (d, J=7.5 Hz, 1H), 4.93 (s, 2H), 4.69 (s, 2H), 4.08 (s, 2H), 3.60-3.51 (m, 2H), 3.35-3.29 (m, 6H), 3.22 (s, 4H), 1.15 (dd, J=13.7, 6.7 Hz, 9H).

### Example 47

### Synthesis of Compound 154

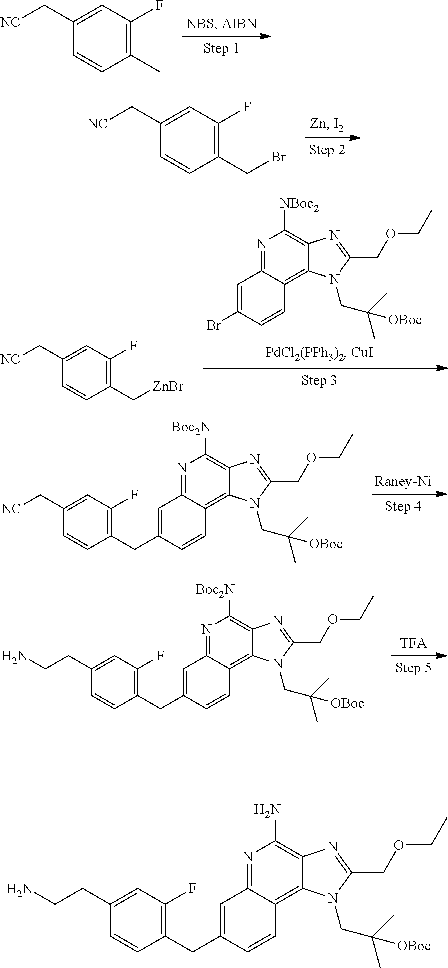

Synthetic procedures for Compound 154 were similar with those of Compound 1.

MS (ESI) calcd for C₂₆H₃₂FN₅O₂[M+H]⁺ m/z=466.25, found 466.20.

¹H NMR (400 MHz, DMSO-d6) δ 13.80 (s, 1H), 9.06 (s, 2H), 8.43 (d, J=8.6 Hz, 1H), 7.86 (s, 3H), 7.57 (s, 1H), 7.37 (t, J=7.7 Hz, 2H), 7.15 (d, J=11.0 Hz, 1H), 7.08 (d, J=7.6 Hz, 1H), 4.93 (s, 2H), 4.69 (s, 2H), 4.13 (s, 2H), 3.54 (q, J=6.9 Hz, 2H), 3.05 (s, 2H), 2.85 (t, J=7.6 Hz, 2H), 1.14 (t, J=7.0 Hz, 9H).

### Example 48

### Synthesis of Compound 155

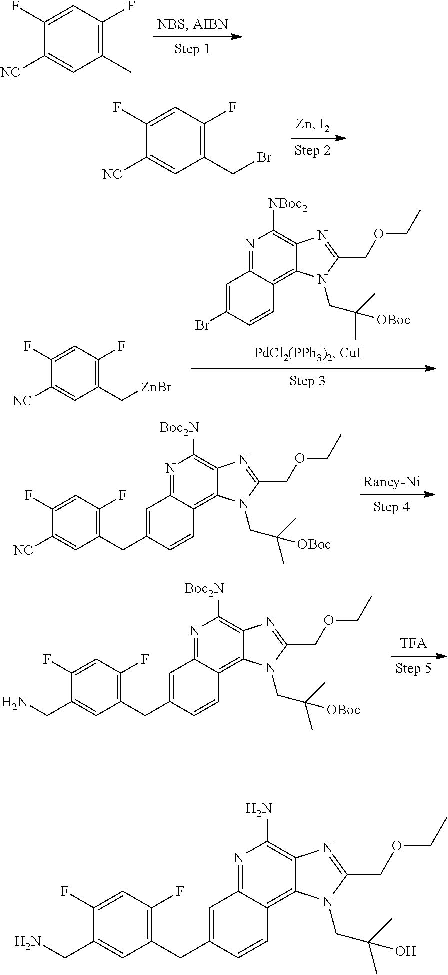

Synthetic procedures for Compound 155 were similar with those of Compound 1.

MS (ESI) calcd for C₂₅H₂₉F₂N₅O₂[M+H]⁺ m/z=470.23, found 470.20.

### Example 49

### Synthesis of Compound 156

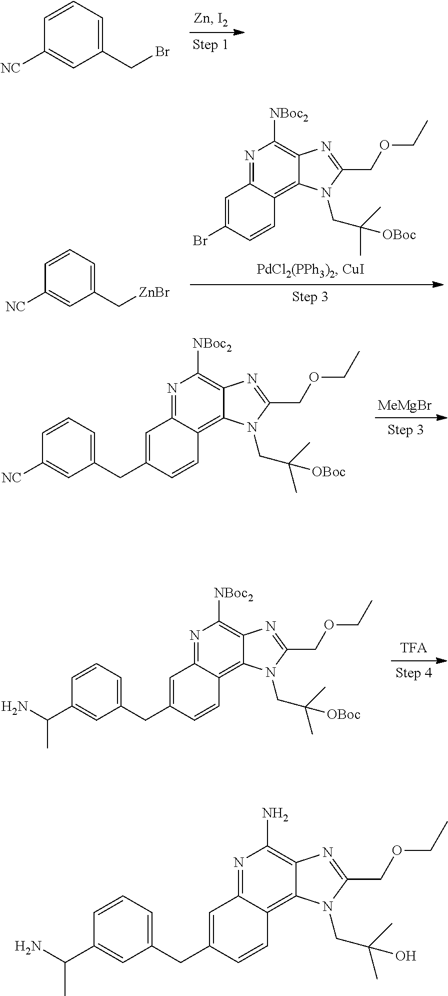

### Step 1. Preparation of (3-cyanobenzyl)zinc(II) bromide

A mixture of Zn (1.75 g, 26.8 mmol) and I₂ (0.15 g, 0.59 mmol) in N₂ protection was stirred 5 minutes under heat. Then 3-(bromomethyl)benzonitrile (908 mg, 4.46 mmol) in DMF (6 mL) was added to the mixture under heat. Then the mixture was stirred for 5 minutes and used in the next step.

### Step 2. Preparation of tert-butyl(tert-butoxycarbonyl)(1-(2-((tert-butoxycarbonyl)oxy)-2-methylpropyl)-7-(3-cyanobenzyl)-2-(ethoxymethyl)-1H-imidazo[4,5-c]quinolin-4-yl)carbamate

To a solution of tert-butyl(7-bromo-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate (297 mg, 0.43 mmol), PdCl₂(PPh₃)₂(120 mg, 0.17 mmol) and CuI (100 mg, 0.53 mmol) in DMF (3 mL) was added 3-(bromomethyl)benzonitrile zinc(II) bromide (5.5 mL) under N₂. Then the mixture was stirred at 50° C. for 1 hour. TLC (n-Heptane:EA=1:1) showed the reaction was OK. It was poured into saturated NH₄Cl (aq.) (40 mL) and extracted with EA (30 mL*2), the combined organic phases were washed with water (20 mL*2) and dried over anhydrous Na₂SO₄, concentrated on vacuum to give the crude. The residue was purified by silica gel column chromatography (eluting with PE:EA=3:1) to give the product (303 mg, 96.8% yield) as a yellow oil.

LCMS (ESI) calcd for C₄₀H₅₁N₅O₈ [M+H]⁺ m/z=730.37, found 730.4.

### Step 3. Preparation of tert-butyl(7-(3-(1-aminoethyl)benzyl)-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate

A solution of tert-butyl(tert-butoxycarbonyl)(1-(2-((tert-butoxycarbonyl)oxy)-2-methylpropyl)-7-(3-cyanobenzyl)-2-(ethoxymethyl)-1H-imidazo[4,5-c]quinolin-4-yl)carbamate (40 mg, 0.056 mmol) in anhydrous THF (2 mL) was stirred under hydrogen (1 atm) at room temperature. The mixture was added methylmagnesium bromide (3M in THF) (80 uL) at 0° C. Then the reaction mixture was stirred at 60° C. for 2 hours. The reaction was cooled to 0° C. and added LiBH₄ (400 mg, 18.37 mmol). Then the reaction mixture was stirred at 60° C. for 1 hour. HPLC showed the reaction was OK. The mixture was quenched with saturated NH₄Cl (aq.) (20 mL) and extracted with EA (10 mL*2), the combined organic phases were washed with water (10 mL*2) and dried over anhydrous Na₂SO₄, concentrated on vacuum to give the crude.

LCMS (ESI) calcd for C₄₁H₅₇N₅O₈ [M+H]⁺ m/z=748.42, found 648.4, 748.9.

### Step 5. Preparation of 1-(4-amino-7-(3-(1-aminoethyl)benzyl)-2-(ethoxymethyl)-1H-imidazo[4,5-c]quinolin-1-yl)-2-methylpropan-2-ol

To a solution of tert-butyl(7-(3-(1-aminoethyl)benzyl)-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate (100 mg, crude) in DCM (2 mL) was added TFA (1 mL) dropwise at room temperature. The reaction mixture was stirred at room temperature for 3 hours. HPLC showed the reaction was OK. The solution was removed under reduced pressure to afford the crude product. The residue was purified by Prep-HPLC (column: Gemini-C18 250×21.2 mm, 10 um; mobile phase: ACN-H₂O (0.1% TFA); gradient: 20%-65%) to afford Compound 156 (5.75 mg, 10.4% yield) as white solid.

MS (ESI) calcd for C₂₆H₃₃N₅O₂ [M+H]⁺ m/z=448.26, found 448.4.

### Example 50

### Synthesis of Compound 157

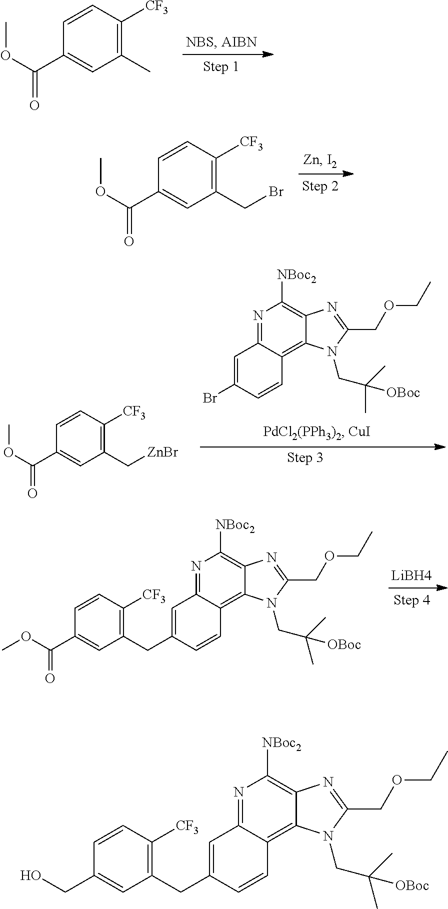

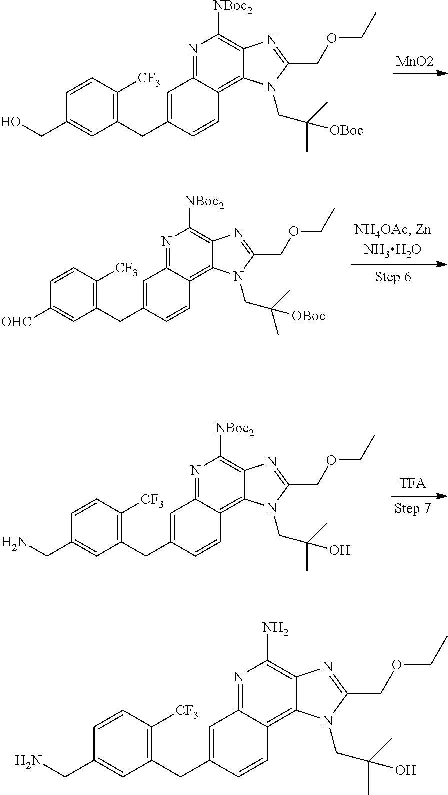

### Step 1. Preparation of methyl 3-(bromomethyl)-4-(trifluoromethyl)benzoate

To a solution of 2,4-difluoro-5-methylbenzonitrile (4.0 g, 18.3 mmol) in CCl₄ (100 mL) was added NBS (8.0 g, 18.3 mmol) and AIBN (0.33 g, 1.83 mmol) at room temperature. The mixture was stirred at 80° C. for 3 hours. HPLC showed the reaction was completed. The reaction was diluted with EA and filtered. The filtrated was concentrated in vacuo to give the crude. The residue was purified by Prep-HPLC (Column: Gemini-C18, 250×50 mm, 10 um; Mobile Phase: ACN-H₂O (0.1% TFA), Gradient: 10%-65%) to give the product (2.10 g, 38.53% yield) as a white solid.

¹H NMR (400 MHz, CDCl₃) δ 8.26 (s, 1H), 8.06 (d, J=8.2 Hz, 1H), 7.73 (d, J=8.2 Hz, 1H), 4.65 (s, 2H), 3.97 (s, 3H).

### Step 2. Preparation of (5-(methoxycarbonyl)-2-(trifluoromethyl)benzyl)zinc(II) bromide

A mixture of Zn (1.75 g, 26.8 mmol) and 12 (0.15 g, 0.59 mmol) in N₂ protection was stirred 5 minutes under heat. Then methyl 3-(bromomethyl)-4-(trifluoromethyl)benzoate (1.93 g, 4.46 mmol) in DMF (12 mL) was added to the mixture under heat. Then the mixture was stirred for 5 minutes and used in the next step.

### Step 3. Preparation of methyl3-((4-(bis(tert-butoxycarbonyl)amino)-1-(2-((tert-butoxycarbonyl)oxy)-2-methylpropyl)-2-(ethoxymethyl)-1H-imidazo[4,5-c]quinolin-7-yl)methyl)-4-(trifluoromethyl)benzoate

To a solution of tert-butyl(7-bromo-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate (300 mg, 0.433 mmol), PdCl₂(PPh₃)₂(120 mg, 0.17 mmol) and CuI (100 mg, 0.53 mmol) in DMF (3 mL) was added (5-(methoxycarbonyl)-2-(trifluoromethyl)benzyl)zinc(II) bromide (5.5 mL) under N₂. Then the mixture was stirred at 50° C. for 1 hour. TLC (n-Heptane:EA=1:1) showed the reaction was OK. It was poured into saturated NH₄Cl (aq.) (40 mL) and extracted with EA (30 mL*2), the combined organic phases were washed with water (20 mL*2) and dried over anhydrous Na₂SO₄, concentrated on vacuum to give the crude. The residue was purified by silica gel column chromatography (eluting with PE:EA=3:1) to give the product (280 mg, 78.1% yield) as a yellow oil.

LCMS (ESI) calcd for C₄₂H₅₃F₃N₄O₁₀ [M+H]⁺ m/z=831.37, found 831.6.

### Step 4. Preparation of tert-butyl(tert-butoxycarbonyl)(1-(2-((tert-butoxycarbonyl)oxy)-2-methylpropyl)-2-(ethoxymethyl)-7-(5-(hydroxymethyl)-2-(trifluoromethyl)benzyl)-1H-imidazo[4,5-c]quinolin-4-yl)carbamate

A solution of tert-butyl (tert-butoxycarbonyl)(1-(2-((tert-butoxycarbonyl)oxy)-2-methylpropyl)-7-(5-cyano-2,4-difluorobenzyl)-2-(ethoxymethyl)-1H-imidazo[4,5-c]quinolin-4-yl)carbamate (270 mg, 0.325 mmol) in MeOH (8 mL) was added LiBH₄(23.4 mg, 0.975 mmol). The reaction mixture was stirred at room temperature for 3 hours. TLC showed the reaction was OK. The mixture was diluted with water and concentrated to give the crude product. The residue was purified by silica gel column chromatography (eluting with PE:EA=5:1) to give the product (280 mg, 100% yield) as a yellow oil.

LCMS (ESI) calcd for C₄₁H₅₃F₃N₄O₉[M+H]⁺ m/z=803.38, found 803.6.

### Step 5. Preparation of tert-butyl (tert-butoxycarbonyl)(J-(2-((tert-butoxycarbonyl)oxy)-2-methylpropyl)-2-(ethoxymethyl)-7-(5-formyl-2-(trifluoromethyl)benzyl)-1H-imidazo[4,5-c]quinolin-4-yl)carbamate

To a solution of tert-butyl(tert-butoxycarbonyl)(1-(2-((tert-butoxycarbonyl)oxy)-2-methylpropyl)-2-(ethoxymethyl)-7-(5-(hydroxymethyl)-2-(trifluoromethyl)benzyl)-1H-imidazo[4,5-c]quinolin-4-yl)carbamate (270 mg, 0.337 mmol) in DCM (5 mL) was added MnO₂ (146.4 mg, 1.683 mmol). The reaction mixture was stirred at room temperature for 12 hours. TLC showed the reaction was OK. The reaction was diluted with EA and filtered. The filtrated was concentrated in vacuo to give the crude. The residue was purified by silica gel column chromatography (eluting with PE:EA=5:1) to give the product (220 mg, 81.48% yield) as a white solid.

LCMS (ESI) calcd for C₄₁H₅₁F₃N₄O₉[M+H]⁺ m/z=801.36, found 801.6, 701.5.

### Step 6. Preparation of tert-butyl (7-(5-(aminomethyl)-2-(trifluoromethyl)benzyl)-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate

To a solution of tert-butyl(tert-butoxycarbonyl)(1-(2-((tert-butoxycarbonyl)oxy)-2-methylpropyl)-2-(ethoxymethyl)-7-(5-formyl-2-(trifluoromethyl)benzyl)-1H-imidazo[4,5-c]quinolin-4-yl)carbamate (270 mg, 0.338 mmol) in EtOH (2 mL) and H₂O (2 mL) was added NH₄OAc (78.08 mg, 1.014 mmol). The reaction mixture was stirred at room temperature for 2 hours. To the mixture was added Zn (109.9 mg, 1.69 mmol) and NH₃·H₂O (2.3 mg, 0.067 mmol) and stirred for 3 hours. TLC showed the reaction was OK. The reaction was filtered. The filtrated was concentrated in vacuo to give the crude. The residue was purified by silica gel column chromatography (eluting with PE:EA=5:1) to give the product (150 mg, 55.56% yield) as a white solid.

LCMS (ESI) calcd for C₄₁H₅₄F₃N₅O₈ [M+H]⁺ m/z=802.39, found 702.5.

### Step 7. Preparation of 1-(4-amino-7-(5-(aminomethyl)-2-(trifluoromethyl)benzyl)-2-(ethoxymethyl)-1H-imidazo[4,5-c]quinolin-1-yl)-2-methylpropan-2-ol

To a solution of tert-butyl (7-(5-(aminomethyl)-2-(trifluoromethyl)benzyl)-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate (150 mg, 0.187 mmol) in DCM (2 mL) was added TFA (1 mL) drop wise at room temperature. The reaction mixture was stirred at room temperature for 3 hours. HPLC showed the reaction was OK. The solution was removed under reduced pressure to afford the crude product. The residue was purified by Prep-HPLC (column: Gemini-C18 250*21.2 mm, 10 um; mobile phase: ACN-H₂O (0.1% TFA); gradient: 20%-65%) to afford Compound 157 (78.05 mg, 83.2% yield) as white solid.

MS (ESI) calcd for C₂₆H₃₀F₃N₅O₂[M+H]⁺ m/z=502.55, found 502.3.

¹H NMR (400 MHz, DMSO-d6) δ 13.78 (s, 1H), 9.03 (s, 2H), 8.44 (d, J=8.7 Hz, 1H), 8.31 (s, 3H), 7.88 (d, J=8.2 Hz, 1H), 7.65-7.55 (m, 2H), 7.49 (s, 1H), 7.30 (d, J=8.6 Hz, 1H), 4.94 (s, 2H), 4.69 (s, 2H), 4.33 (s, 2H), 4.12 (d, J=4.9 Hz, 2H), 1.15 (dd, J=14.5, 7.5 Hz, 9H).

### Example 51

### Synthesis of Compound 158

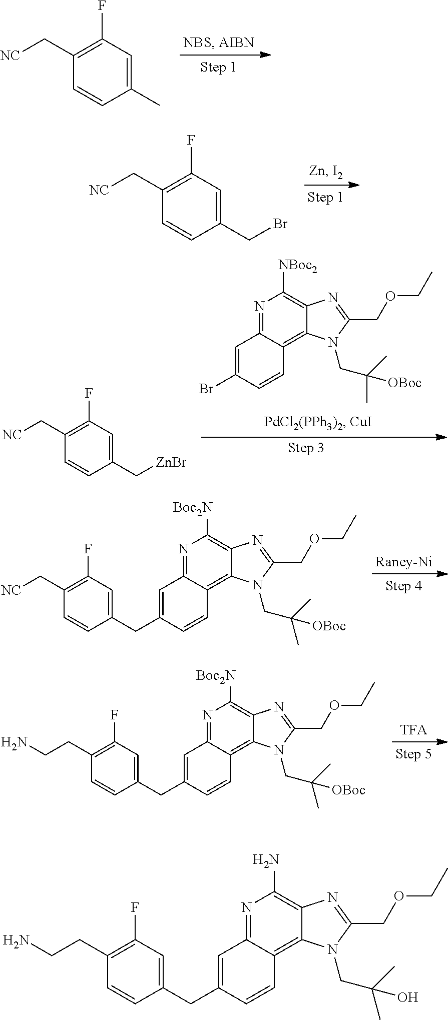

### Step 1. Preparation of 2-(4-(bromomethyl)-2-fluorophenyl)acetonitrile

To a solution of 2-(2-fluoro-4-methylphenyl)acetonitrile (3.80 g, 25.5 mmol) in CCl₄ (100 mL) was added NBS (8.0 g, 25.5 mmol) and AIBN (0.33 g, 2.55 mmol) at room temperature. The mixture was stirred at 80° C. for 3 hours. HPLC showed the reaction was completed. The reaction was diluted with EA and filtered. The filtrated was concentrated in vacuo to give the crude. The residue was purified by Prep-HPLC (Column: Gemini-C18, 250×50 mm, 10 um; Mobile Phase:ACN-H₂O (0.1% TFA), Gradient: 10%-65%) to give the product (850.1 mg g, 14.6% yield) as a white solid.

¹H NMR (400 MHz, CDCl₃) δ 7.43 (t, J=7.8 Hz, 1H), 7.22 (d, J=7.9 Hz, 1H), 7.16 (dd, J=10.2, 1.4 Hz, 1H), 4.44 (s, 2H), 3.76 (s, 2H).

### Step 2. Preparation of (4-(cyanomethyl)-3-fluorobenzyl)zinc(II) bromide

A mixture of Zn (1.75 g, 26.8 mmol) and I₂ (0.15 g, 0.59 mmol) in N₂ protection was stirred 5 minutes under heat. Then 2-(4-(bromomethyl)-2-fluorophenyl) acetonitrile (850 mg, 4.46 mmol) in DMF (6 mL) was added to the mixture under heat. Then the mixture was stirred for 5 minutes and used in the next step.

### Step 3. Preparation of tert-butyl(tert-butoxycarbonyl)(1-(2-((tert-butoxycarbonyl)oxy)-2-methylpropyl)-7-(4-(cyanomethyl)-3-fluorobenzyl)-2-(ethoxymethyl)-1H-imidazo[4,5-c]quinolin-4-yl)carbamate

To a solution of tert-butyl(7-bromo-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate (303 mg, 0.44 mmol), PdCl₂(PPh₃)₂(120 mg, 0.17 mmol) and CuI (100 mg, 0.53 mmol) in DMF (3 mL) was added (4-(cyanomethyl)-3-fluorobenzyl)zinc(II) bromide (5.5 mL) under N₂. Then the mixture was stirred at 50° C. for 1 hour. TLC (n-Heptane:EA=1:1) showed the reaction was OK. It was poured into saturated NH₄Cl (aq.) (40 mL) and extracted with EA (30 mL*2), the combined organic phases were washed with water (20 mL*2) and dried over anhydrous Na₂SO₄, concentrated on vacuum to give the crude. The residue was purified by silica gel column chromatography (eluting with PE:EA=3:1) to give the product (400 mg, 95.3% yield) as a yellow oil.

LCMS (ESI) calcd for C₄₁H₅₂FN₅O₈ [M+H]⁺ m/z=762.38, found 762.7.

### Step 4. Preparation of tert-butyl(7-(4-(2-aminoethyl)-3-fluorobenzyl)-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate

A solution of tert-butyl (tert-butoxycarbonyl)(1-(2-((tert-butoxycarbonyl)oxy)-2-methylpropyl)-7-(4-(cyanomethyl)-3-fluorobenzyl)-2-(ethoxymethyl)-1H-imidazo[4,5-c]quinolin-4-yl)carbamate (400 mg, 0.52 mmol) and Raney-Ni (about 1 g) in NH₃-MeOH (7M) (20 mL) was stirred under hydrogen (1 atm) at room temperature. The reaction mixture was stirred at room temperature for 2 hours. HPLC showed the reaction was OK. The mixture was filtered through Celite and the filtrate was concentrated to give the crude product (360 mg, 89.5% yield).

LCMS (ESI) calcd for C₄₁H₅₆FN₅O₈ [M+H]⁺ m/z=766.41, found 766.6.

### Step 5. Preparation of 1-(4-amino-7-(4-(2-aminoethyl)-3-fluorobenzyl)-2-(ethoxymethyl)-1H-imidazo[4,5-c]quinolin-1-yl)-2-methylpropan-2-ol

To a solution of tert-butyl (7-(4-(2-aminoethyl)-3-fluorobenzyl)-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate (360 mg, 0.47 mmol) in DCM (2 mL) was added TFA (1 mL) drop wise at room temperature. The reaction mixture was stirred at room temperature for 3 hours. HPLC showed the reaction was OK. The solution was removed under reduced pressure to afford the crude product. The residue was purified by Prep-HPLC (column: Gemini-C18 250×21.2 mm, 10 um; mobile phase: ACN-H₂O (0.10% TFA); gradient: 20%-65%) to afford Compound 158 (156.5 mg, 71.29% yield) as white solid.

MS (ESI) calcd for C₂₆H₃₂FN₅O₂[M+H]⁺ m/z=466.25, found 466.3.

¹H NMR (400 MHz, DMSO-d6) δ 13.80 (s, 1H), 9.08 (s, 2H), 8.43 (d, J=8.6 Hz, 1H), 7.90 (s, 3H), 7.57 (s, 1H), 7.39 (d, J=8.7 Hz, 1H), 7.29 (t, J=7.9 Hz, 1H), 7.14 (dd, J=17.8, 9.5 Hz, 2H), 4.94 (s, 2H), 4.71 (s, 2H), 4.14 (s, 2H), 3.54 (q, J=7.0 Hz, 2H), 3.00 (dd, J=9.0, 5.4 Hz, 2H), 2.91-2.82 (m, 2H), 1.15 (dd, J=13.9, 6.9 Hz, 9H).

### Example 52

### Synthesis of Compound 159

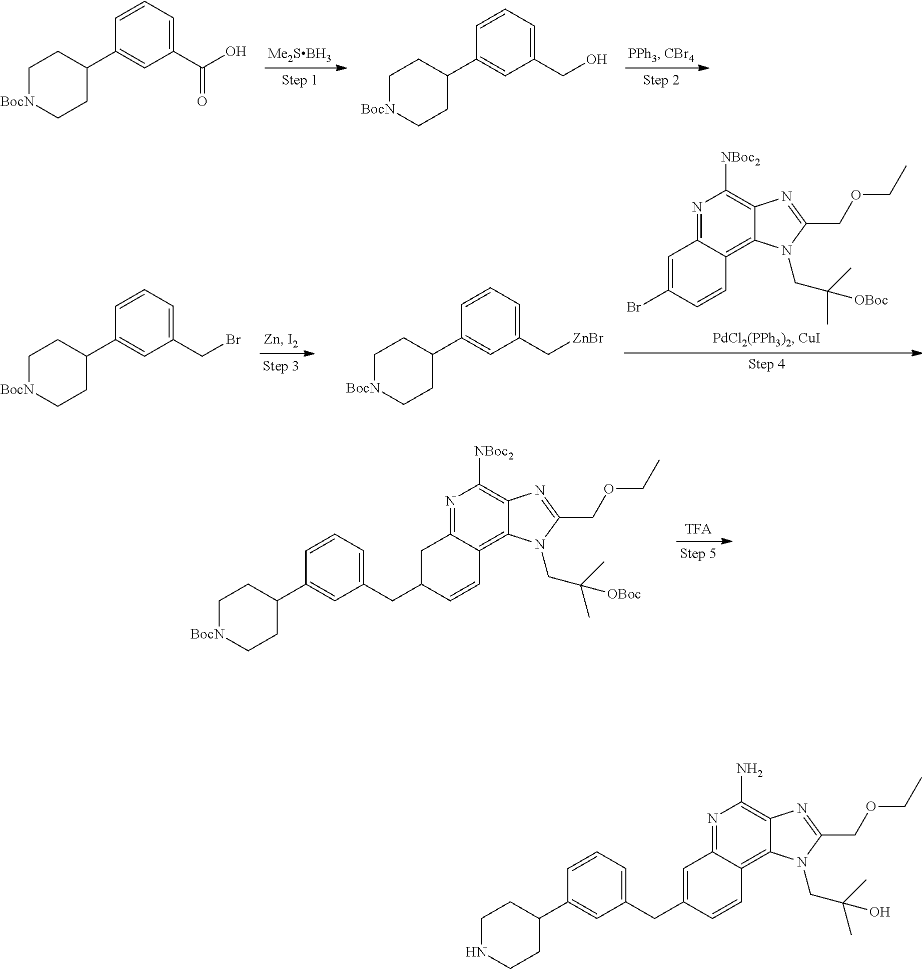

### Step 1. Preparation of tert-butyl 4-(3-(hydroxymethyl) phenyl) piperidine-1-carboxylate

To a solution of 3-(1-(tert-butoxycarbonyl) piperidin-4-yl) benzoic acid (2.0 g, 6.5494 mmol) in THF (40 mL) was added Me₂S·BH₃ (7.4 mL) drop wise at 0° C. under N₂. Then the mixture was stirred at 35° C. for 3 hours. TLC (n-Heptane:EA=1:1) showed the reaction was OK. It was poured into saturated NH₄Cl (aq.) (100 mL) and extracted with EA (100 mL*3), the combined organic phases were washed with saturated NaCl (aq.) (80 mL*2) and dried over anhydrous Na₂SO₄, concentrated in vacuo to give the crude. The residue was purified by silica gel column chromatography (eluting with PE:EA=4:1) to give the product (1.90 g, 99% yield) as a yellow oil.

¹H NMR (400 MHz, CDCl₃) δ 7.31 (t, J=7.9 Hz, 1H), 7.21 (d, J=6.7 Hz, 2H), 7.14 (d, J=7.6 Hz, 1H), 4.69 (s, 2H), 4.24 (d, J=12.2 Hz, 2H), 2.80 (t, J=12.4 Hz, 2H), 2.65 (tt, J=12.2, 3.5 Hz, 1H), 1.82 (d, J=13.2 Hz, 2H), 1.66 (dd, J=12.3, 4.2 Hz, 3H), 1.48 (s, 9H).

### Step 2. Preparation of tert-butyl 4-(3-(bromomethyl) phenyl) piperidine-1-carboxylate

To a solution of tert-butyl 4-(3-(hydroxymethyl) phenyl) piperidine-1-carboxylate (2.35 g, 8.0648 mmol) in DCM (50 mL) was added PPh₃ (2.33 g, 8.8713 mmol) and imidazole (658.8 mg, 9.6777 mmol) under N₂. The mixture was added CBr₄ (2.94 g, 8.8713 mmol) drop wise at 0° C. Then the mixture was stirred at 0° C. for 1 hour. TLC (n-Heptane:EA=2:1) showed the reaction was OK. The reaction mixture was diluted with H₂O (100 mL) and extracted with EA (100 mL*3), the combined organic phases were washed with saturated NaCl (aq.) (100 mL*2) and dried over anhydrous Na₂SO₄, concentrated in vacuo to give the crude. The residue was purified by silica gel column chromatography (eluting with PE:EA=9:1) to give the product (1.80 g, 62.9% yield) as yellow oil.

### Step 3. Preparation of (3-(1-(tert-butoxycarbonyl)piperidin-4-yl)benzyl)zinc(II) bromide

A mixture of Zn (1.75 g, 26.8 mmol) and I₂ (0.15 g, 0.59 mmol) in N₂ protection was stirred 5 minutes under heat. Then tert-butyl 4-(3-(bromomethyl)phenyl)piperidine-1-carboxylate) (1.15 g, 4.46 mmol) in DMF (6 mL) was added to the mixture under heat. Then the mixture was stirred for 5 minutes and used in the next step.

### Step 4. Preparation of tert-butyl 4-(3-((4-(bis(tert-butoxycarbonyl)amino)-1-(2-((tert-butoxycarbonyl)oxy)-2-methylpropyl)-2-(ethoxymethyl)-1H-imidazo[4,5-c]quinolin-7-yl)methyl)phenyl)piperidine-1-carboxylate

To a solution of tert-butyl(7-bromo-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate (151 mg, 0.215 mmol), PdCl₂(PPh₃)₂(120 mg, 0.17 mmol) and CuI (100 mg, 0.53 mmol) in DMF (3 mL) was added (3-(1-(tert-butoxycarbonyl)piperidin-4-yl)benzyl)zinc(II) bromide (5.5 mL) under N₂. Then the mixture was stirred at 50° C. for 1 hour. HPLC showed the reaction was OK. It was poured into saturated NH₄Cl (aq.) (40 mL) and extracted with EA (30 mL*2), the combined organic phases were washed with water (20 mL*2) and dried over anhydrous Na₂SO₄, concentrated in vacuum to give the crude. The residue was purified by silica gel column chromatography (eluting with PE:EA=3:1) to give the product (190 mg, 98.9% yield) as a yellow oil.

MS (ESI) calcd for C₄₉H₆₉N₅O₁₀ [M+H]⁺ m/z=888.50, found 888.7.

### Step 5. Preparation of 1-(4-amino-2-(ethoxymethyl)-7-(3-(piperidin-4-yl)benzyl)-1H-imidazo[4,5-c]quinolin-1-yl)-2-methylpropan-2-ol

To a solution of tert-butyl 4-(3-((4-(bis(tert-butoxycarbonyl)amino)-1-(2-((tert-butoxycarbonyl)oxy)-2-methylpropyl)-2-(ethoxymethyl)-1H-imidazo[4,5-c]quinolin-7-yl)methyl)phenyl)piperidine-1-carboxylate (180 mg, 0.2027 mmol) in DCM (2 mL) was added TFA (1 mL) drop wise at room temperature. The reaction mixture was stirred at room temperature for 3 hours. HPLC showed the reaction was OK. The solution was removed under reduced pressure to afford the crude product. The residue was purified by Prep-HPLC (column: Gemini-C18 250×21.2 mm, 10 um; mobile phase: ACN-H₂O (0.1% TFA); gradient: 20%-65%) to afford Compound 159 (88.1 mg, 89.0% yield) as white solid.

MS (ESI) calcd for C₂₉H₃₇N₅O₂ [M+H]⁺ m/z=488.29, found 488.3.

¹H NMR (400 MHz, DMSO-d6) δ 13.79 (s, 1H), 9.00 (s, 2H), 8.66 (s, 1H), 8.42 (d, J=8.6 Hz, 2H), 7.56 (s, 1H), 7.36 (d, J=8.6 Hz, 1H), 7.30 (t, J=7.6 Hz, 1H), 7.15 (d, J=7.5 Hz, 2H), 7.10 (d, J=7.6 Hz, 1H), 4.94 (s, 2H), 4.70 (s, 2H), 4.13 (s, 2H), 3.54 (q, J=7.0 Hz, 2H), 2.98 (dd, J=23.3, 11.8 Hz, 2H), 2.81 (t, J=12.1 Hz, 1H), 1.91 (d, J=12.9 Hz, 2H), 1.76 (qd, J=13.3, 3.6 Hz, 2H), 1.36-1.01 (m, 9H).

### Example 53

### Synthesis of Compound 160

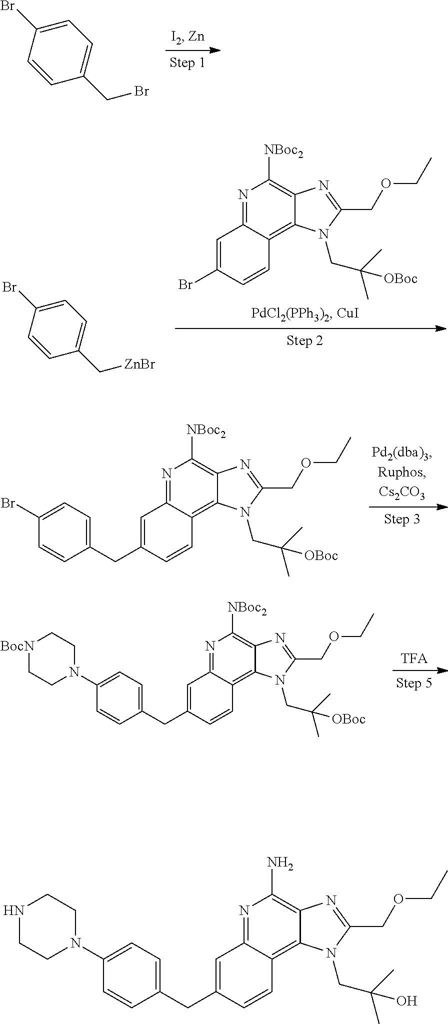

### Step 1. Preparation of (4-bromobenzyl)zinc(II) bromide

A mixture of Zn (1.75 g, 26.8 mmol) and I₂ (0.15 g, 0.59 mmol) in N₂ protection was stirred 5 minutes under heat. Then 1-bromo-4-(bromomethyl)benzene) (975 mg, 4.46 mmol) in DMF (6 mL) was added to the mixture under heat. Then the mixture was stirred for 5 minutes and used in the next step.

### Step 2. Preparation of tert-butyl(7-(4-bromobenzyl)-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate

To a solution of tert-butyl(7-bromo-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate (302 mg, 0.44 mmol), PdCl₂(PPh₃)₂(120 mg, 0.17 mmol) and CuI (100 mg, 0.53 mmol) in DMF (3 mL) was added (4-bromobenzyl)zinc(II) bromide (5.5 mL) under N₂. Then the mixture was stirred at 50° C. for 1 hour. HPLC showed the reaction was OK. It was poured into saturated NH₄Cl (aq.) (40 mL) and extracted with EA (30 mL*2), the combined organic phases were washed with water (20 mL*2) and dried over anhydrous Na₂SO₄, concentrated in vacuo to give the crude. The residue was purified by silica gel column chromatography (eluting with PE:EA=3:1) to give the product (180 mg, 59.6% yield) as a yellow oil.

LCMS (ESI) calcd for C₃₉H₅₁BrN₄O₈ [M+H]⁺ m/z=783.29, found 783.5.

### Step 3. Preparation of tert-butyl 4-(4-((4-(bis(tert-butoxycarbonyl)amino)-1-(2-((tert-butoxycarbonyl)oxy)-2-methylpropyl)-2-(ethoxymethyl)-1H-imidazo[4,5-c]quinolin-7-yl)methyl)phenyl)piperazine-1-carboxylate

To a solution of tert-butyl (7-(4-bromobenzyl)-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate (130 mg, 0.1658 mmol), tert-butyl piperazine-1-carboxylate (92.6 mg, 0.4976 mmol), Pd₂(dba)₃ (60 mg, 0.0663 mmol) and Ruphos (61 mg, 0.1326 mmol) in DMF (6 mL) was added Cs₂CO₃ (162 mg, 0.4974 mmol) under N₂. Then the mixture was stirred at 90° C. for 2 hours. TLC (n-Heptane:EA=1:1) showed the reaction was OK. The reaction mixture was diluted with H₂O (80 mL) and extracted with EA (60 mL*2), the combined organic phases were washed with sat. NaCl aq. (50 mL*2) and dried over anhydrous Na₂SO₄, concentrated in vacuo to give the crude. The residue was purified by Prep-HPLC (Column: Gemini-C18, 250×21.2 mm, 10 um; Mobile Phase: ACN-H₂O (0.1% TFA), Gradient: 20%-65%) to afford product (90 mg, 52.9% yield) as yellow solid.

LCMS (ESI) calcd for C₄₈H₆₈N₆O₁₀ [M+H]⁺ m/z=889.50, found 889.6.

### Step 4. Preparation of 1-(4-amino-2-(ethoxymethyl)-7-(4-(piperazin-1-yl)benzyl)-1H-imidazo[4,5-c]quinolin-1-yl)-2-methylpropan-2-ol

To a solution of tert-butyl 4-(4-((4-(bis(tert-butoxycarbonyl)amino)-1-(2-((tert-butoxycarbonyl)oxy)-2-methylpropyl)-2-(ethoxymethyl)-1H-imidazo[4,5-c]quinolin-7-yl)methyl)phenyl)piperazine-1-carboxylate (85 mg, 0.0956 mmol) in DCM (2 mL) was added TFA (1 mL) drop wise at room temperature. The reaction mixture was stirred at room temperature for 3 hours. HPLC showed the reaction was OK. The solution was removed under reduced pressure to afford the crude product. The residue was purified by Prep-HPLC (column: Gemini-C18 250×21.2 mm, 10 um; mobile phase: ACN-H₂O (0.1% TFA); gradient: 20%-65%) to afford Compound 160 as white solid.

MS (ESI) calcd for C₂₈H₃₆N₆O₂ [M+H]⁺ m/z=489.29, found 489.3.

¹H NMR (400 MHz, DMSO-d6) δ 13.57 (s, 1H), 8.76 (s, 2H), 8.41 (d, J=8.6 Hz, 1H), 7.55 (s, 1H), 7.35 (d, J=8.6 Hz, 1H), 7.18 (d, J=8.5 Hz, 2H), 6.96 (d, J=8.6 Hz, 2H), 4.94 (s, 2H), 4.70 (s, 2H), 4.04 (s, 2H), 3.54 (q, J=7.0 Hz, 2H), 3.28 (d, J=5.7 Hz, 4H), 3.23 (s, 4H), 1.14 (t, J=7.0 Hz, 9H).

### Example 54

### Synthesis of Compound 161

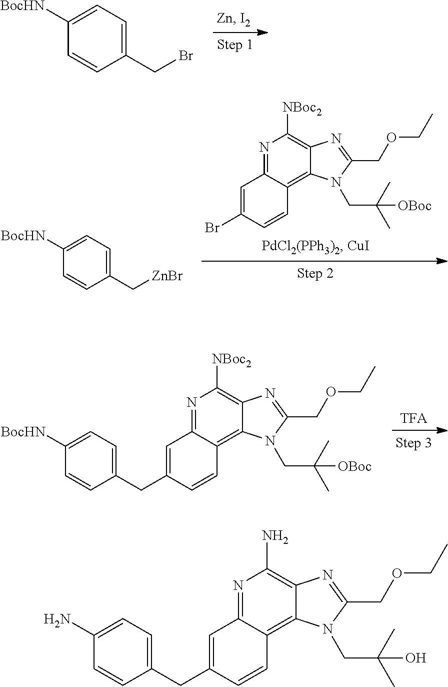

### Step 1. Preparation of (4-((tert-butoxycarbonyl)amino)benzyl)zinc(II) bromide

A mixture of Zn (1.75 g, 26.8 mmol) and 12 (0.15 g, 0.59 mmol) in N₂ protection was stirred 5 minutes under heat. Then tert-butyl (4-(bromomethyl)phenyl)carbamate (960 mg, 3.355 mmol) in DMF (6 mL) was added to the mixture under heat. Then the mixture was stirred for 5 minutes and used in the next step.

### Step 2. Preparation of tert-butyl (tert-butoxycarbonyl)(7-(4-((tert-butoxycarbonyl)amino)benzyl)-1-(2-((tert-butoxycarbonyl)oxy)-2-methylpropyl)-2-(ethoxymethyl)-1H-imidazo[4,5-c]quinolin-4-yl)carbamate

To a solution of tert-butyl(7-bromo-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate (200 mg, 0.2883 mmol), PdCl₂(PPh₃)₂ (120 mg, 0.17 mmol) and CuI (100 mg, 0.53 mmol) in DMF (3 mL) was added (4-((tert-butoxycarbonyl)amino)benzyl)zinc(II) bromide (5.5 mL) under N₂. Then the mixture was stirred at 50° C. for 1 hour. HPLC showed the reaction was OK. It was poured into saturated NH₄Cl (aq) (40 mL) and extracted with EA (30 mL*2), the combined organic phases were washed with water (20 mL*2) and dried over anhydrous Na₂SO₄, concentrated on vacuum to give the crude. The residue was purified by silica gel column chromatography (eluting with PE:EA=3:1) to give the product (220 mg, 84.6% yield) as a yellow oil.

LCMS (ESI) calcd for C₄₄H₆₁N₅O₁₀ [M+H]⁺ m/z=820.44, found 820.5.

### Step 3. Preparation of 1-(4-amino-7-(4-aminobenzyl)-2-(ethoxymethyl)-1H-imidazo[4,5-c]quinolin-1-yl)-2-methylpropan-2-ol

To a solution of tert-butyl (tert-butoxycarbonyl)(7-(4-((tert-butoxycarbonyl)amino)benzyl)-1-(2-((tert-butoxycarbonyl)oxy)-2-methylpropyl)-2-(ethoxymethyl)-1H-imidazo[4,5-c]quinolin-4-yl)carbamate (200 mg, 0.2439 mmol) in DCM (2 mL) was added TFA (1 mL) drop wise at room temperature. The reaction mixture was stirred at room temperature for 3 hours. HPLC showed the reaction was OK. The solution was removed under reduced pressure to afford the crude product. The residue was purified by Prep-HPLC (column: Gemini-C18 250×21.2 mm, 10 um; mobile phase: ACN-H₂O (0.1% TFA); gradient: 20%-65%) to afford 1-(4-amino-7-(4-aminobenzyl)-2-(ethoxymethyl)-1H-imidazo[4,5-c]quinolin-1-yl)-2-methylpropan-2-ol (100.2 mg, 97.8% yield) as white solid.

MS (ESI) calcd for C₂₄H₂₉N₅O₂ [M+H]⁺ m/z=420.23, found 420.4.

¹H NMR (400 MHz, DMSO-d6) δ 9.03 (s, 2H), 8.42 (d, J=8.6 Hz, 1H), 7.55 (s, 1H), 7.35 (d, J=8.6 Hz, 1H), 7.20 (d, J=8.2 Hz, 2H), 6.97 (d, J=8.1 Hz, 2H), 4.92 (s, 2H), 4.69 (s, 3H), 4.07 (s, 2H), 3.54 (q, J=7.0 Hz, 2H), 1.15 (dd, J=13.4, 6.4 Hz, 9H).

### Example 55

### Synthesis of Compound 162

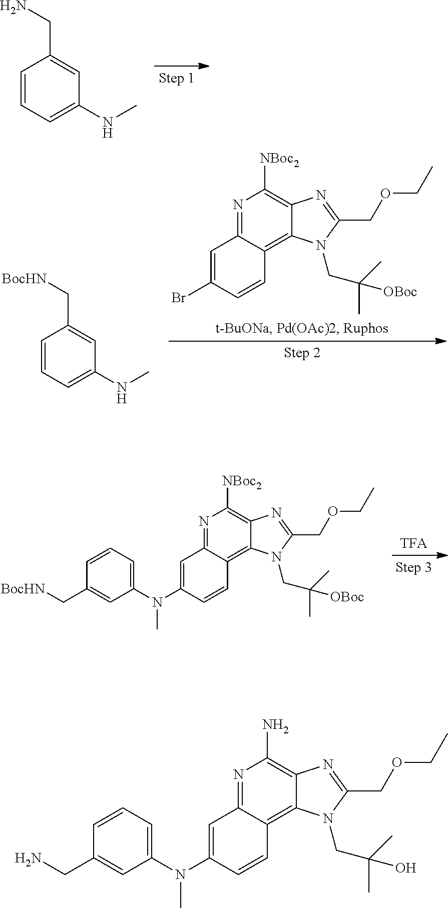

### Step 1. Preparation of tert-butyl(tert-butoxycarbonyl)(1-(2-((tert-tert-butyl (3-(methylamino)benzyl)carbamate

To a solution of 3-(aminomethyl)-N-methylaniline (900 mg, 6.6 mmol) in THF (9 mL) was added di-tert-butyl dicarbonate (1.44 g, 6.6 mmol). The mixture was stirred at room temperature for 4 hours. TLC showed the reaction was completed. The reaction was diluted with EA and filtered. The filtrated was concentrated in vacuo to give the crude. The crude was purified by silica gel column chromatography (eluting with PE:EA=5:1) to give the product (1.50 g, 96.04% yield) as a yellow solid.

LCMS (ESI) calcd for C₁₃H₂₀N₂O₂ [M+H]⁺ m/z=237.15, found 237.4.

### Step 2. Preparation of tert-butyl (tert-butoxycarbonyl)(7-((3-(((tert-butoxycarbonyl)amino)methyl)phenyl)(methyl)amino)-1-(2-((tert-butoxycarbonyl)oxy)-2-methylpropyl)-2-(ethoxymethyl)-1H-imidazo[4,5-c]quinolin-4-yl)carbamate

To a solution of tert-butyl(tert-butoxycarbonyl)(1-(2-((tert-tert-butyl (3-(methylamino)benzyl)carbamate (100 mg, 0.423 mmol) in toluene (5 mL) was added tert-butyl(7-bromo-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate (293.3 mg, 0.423 mmol), t-BuONa (121.9 mg, 1.269 mmol), Pd(OAc)₂ (9.5 mg, 0.0423 mmol, Ruphos (39.4 mg, 0.0846 mmol). The mixture was stirred at 100° C. for 12 hours. TLC showed the reaction was completed. The reaction was diluted with EA and filtered. The filtrated was concentrated in vacuo to give the crude. The crude was purified by silica gel column chromatography (eluting with PE:EA=6:1) to give the product (140 mg, 38.9% yield) as a yellow solid.

LCMS (ESI) calcd for C₄₅H₆₄N₆O₁₀ [M+H]⁺ m/z=849.47, found 749.8.

### Step 3. Preparation of 1-(4-amino-7-((3-(aminomethyl)phenyl)(methyl)amino)-2-(ethoxymethyl)-1H-imidazo[4,5-c]quinolin-1-yl)-2-methylpropan-2-ol

A solution of tert-butyl (tert-butoxycarbonyl)(7-((3-(((tert-butoxycarbonyl)amino)methyl)phenyl)(methyl)amino)-1-(2-((tert-butoxycarbonyl)oxy)-2-methylpropyl)-2-(ethoxymethyl)-1H-imidazo[4,5-c]quinolin-4-yl)carbamate (140 mg, 0.165 mmol) in DCM (2 mL) was added TFA (1 mL) and the mixture was stirred at room temperature for 2 hours. HPLC showed the reaction was OK. The solution was removed under reduced pressure to afford the crude product. The residue was purified by Prep-HPLC (column: Gemini-C18 250×21.2 mm, 10 um; mobile phase: ACN-H₂O (0.1% TFA); gradient: 20%-65%) to afford Compound 162 (71.5 mg, 98.51%, 96.6% yield) as white solid.

MS (ESI) calcd for C₂₅H₃₂N₆O₂ [M+H]⁺ m/z=449.26, found 449.12.

¹H NMR (400 MHz, DMSO-d6) δ 13.45 (s, 1H), 8.86 (s, 2H), 8.26 (d, J=9.0 Hz, 4H), 7.49 (t, J=7.8 Hz, 1H), 7.42 (s, 1H), 7.31 (d, J=6.8 Hz, 2H), 7.13 (s, 1H), 6.97 (d, J=9.1 Hz, 1H), 4.96 (s, 2H), 4.62 (s, 2H), 4.07 (s, 2H), 3.54 (dd, J=13.9, 6.9 Hz, 2H), 1.28-1.03 (m, 9H).

### Example 56

### Synthesis of Compound 163

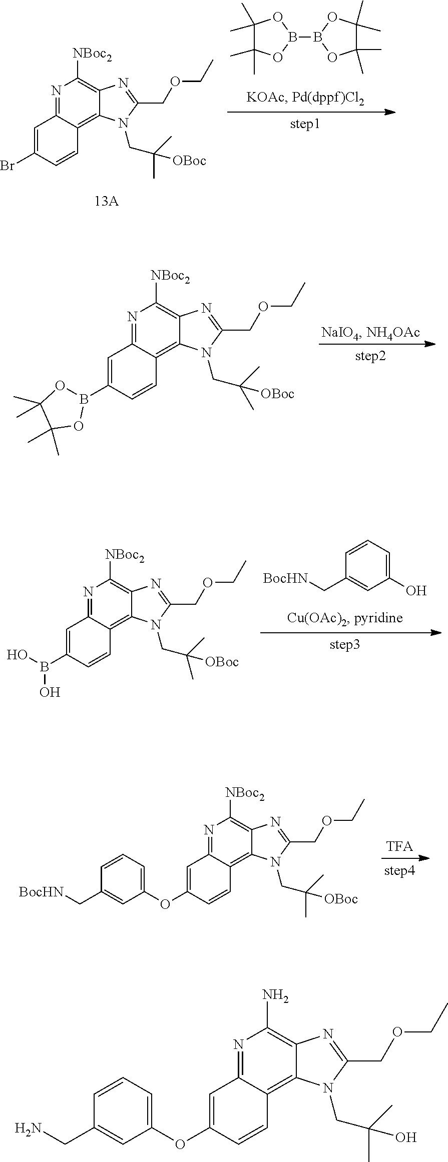

### Step 1. Preparation of tert-butyl(tert-butoxycarbonyl)(1-(2-((tert-butoxycarbonyl)oxy)-2-methylpropyl)-2-(ethoxymethyl)-7-(4,4,5,5-tetramethyl-1,3,2-dioxaborolan-2-yl)-1H-imidazo[4,5-c]quinolin-4-yl)carbamate

To a solution of tert-butyl(7-bromo-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate (200 mg, 0.288 mmol) in dioxane (5 mL) was added bis(pinacolato)diboron (109.9 mg, 0.432 mmol), KOAc (84.8 mg, 0.865 mmol) and Pd(dppf)Cl₂ (21 mg, 0.029 mmol) under N₂. The mixture was stirred at 80° C. for 4 hours. TLC showed the reaction was completed. The reaction was diluted with EA and filtered. The filtrated was concentrated in vacuo to give the crude. The crude was purified by silica gel column chromatography (eluting with PE:EA=5:1) to give the product (180 mg, 86.2% yield) as a yellow solid.

LCMS (ESI) calcd for C₃₈H₅₇BN₄O₁₀ [M+H]⁺ m/z=741.42, found 741.6.

### Step 2. (4-(bis(tert-butoxycarbonyl)amino)-1-(2-((tert-butoxycarbonyl)oxy)-2-methylpropyl)-2-(ethoxymethyl)-1H-imidazo[4,5-c]quinolin-7-yl)boronic acid

To a solution of tert-butyl (tert-butoxycarbonyl)(1-(2-((tert-butoxycarbonyl)oxy)-2-methylpropyl)-2-(ethoxymethyl)-7-(4,4,5,5-tetramethyl-1,3,2-dioxaborolan-2-yl)-1H-imidazo[4,5-c]quinolin-4-yl)carbamate (180 mg, 0.243 mmol) in acetone (2 mL) and H₂O (2 mL) was added Na₁O₄ (155.9 mg, 0.729 mmol), NH₄OAc (37.4 mg, 0.486 mmol). The mixture was stirred at room temperature for 12 hours. TLC showed the reaction was completed. The reaction was diluted with EA and filtered. The filtrated was concentrated in vacuo to give the crude. The crude was purified by silica gel column chromatography (eluting with PE:EA=5:1) to give the product (80 mg, 49.9% yield) as a white solid.

LCMS (ESI) calcd for C₃₂H₄₇BN₄O₁₀ [M+H]⁺ m/z=659.34, found 659.5.

### Step 3. Preparation of tert-butyl (tert-butoxycarbonyl)(7-(3-(((tert-butoxycarbonyl)amino)methyl)phenoxy)-1-(2-((tert-butoxycarbonyl)oxy)-2-methylpropyl)-2-(ethoxymethyl)-1H-imidazo[4,5-c]quinolin-4-yl)carbamate

To a solution of (4-(bis(tert-butoxycarbonyl)amino)-1-(2-((tert-butoxycarbonyl)oxy)-2-methylpropyl)-2-(ethoxymethyl)-1H-imidazo[4,5-c]quinolin-7-yl)boronic acid (54.3 mg, 0.243 mmol), tert-butyl (3-hydroxybenzyl)carbamate (80 mg, 0.122 mmol) and Cu(OAc)₂ (2.44 mg, 0.0122 mmol) in anhydrous DCM (3 mL) was added pyridine (28.9 mg, 0.366 mmol) under N₂. Then the mixture was stirred at 45° C. for 24 hours. TLC (n-Heptane:EA=1:1) showed the reaction was OK. It was concentrated in vacuo to give the crude. The residue was purified by silica gel column chromatography (eluting with PE:EA=2:1) to give the product (80 mg, 78.7% yield) as a yellow oil.

LCMS (ESI) calcd for C₄₄H₆₁N₅O₁₁[M+H]⁺ m/z=836.44, found 836.6.

### Step 4. Preparation of 1-(4-amino-7-(3-(aminomethyl)phenoxy)-2-(ethoxymethyl)-1H-imidazo[4,5-c]quinolin-1-yl)-2-methylpropan-2-ol

A solution of tert-butyl (tert-butoxycarbonyl)(7-(3-(((tert-butoxycarbonyl)amino)methyl)phenoxy)-1-(2-((tert-butoxycarbonyl)oxy)-2-methylpropyl)-2-(ethoxymethyl)-1H-imidazo[4,5-c]quinolin-4-yl)carbamate (80 mg, 0.095 mmol) in DCM (2 mL) was added TFA (1 mL) and the mixture was stirred at room temperature for 2 hours. HPLC showed the reaction was OK. The solution was concentrated in vacuo to afford the crude product. The residue was purified by Prep-HPLC (column: Gemini-C18 250×21.2 mm, 10 um; mobile phase: ACN-H₂O (0.1% TFA); gradient: 20%-65%) to afford Compound 163 (15 mg, 36.03% yield) as white solid. MS (ESI) calcd for C₂₄H₂₉N₅O₃ [M+H]⁺ m/z=436.23, found 436.4.

¹H NMR (400 MHz, DMSO) δ 8.52 (d, J=9.2 Hz, 1H), 8.24 (s, 3H), 7.54 (t, J=7.9 Hz, 1H), 7.39-7.31 (m, 2H), 7.28 (s, 1H), 7.19 (ddd, J=11.3, 8.6, 2.1 Hz, 2H), 4.96 (s, 2H), 4.69 (s, 2H), 4.08 (s, 2H), 3.55 (q, J=7.0 Hz, 2H), 1.16 (dd, J=16.4, 9.4 Hz, 9H).

### Example 57

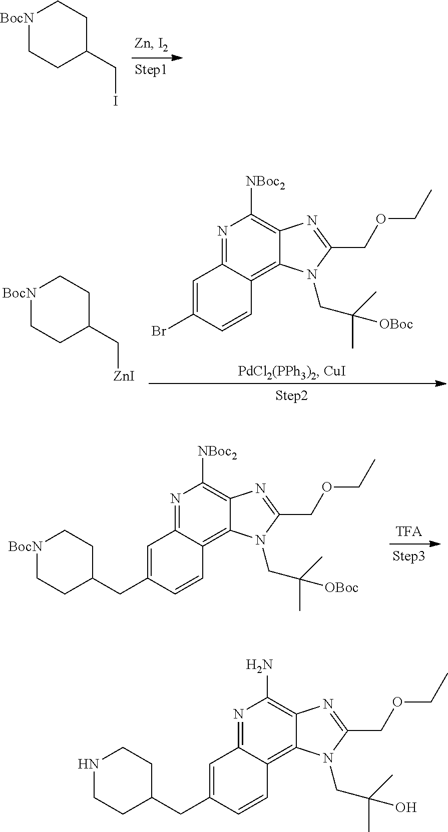

### Step 1. Preparation of ((1-(tert-butoxycarbonyl)piperidin-4-yl)methyl)zinc(II) iodide

A mixture of Zn (1.75 g, 26.8 mmol) and I₂ (0.15 g, 0.59 mmol) in N₂ protection was stirred 5 minutes under heat. Then tert-butyl 4-(iodomethyl)piperidine-1-carboxylate (1.06 g, 4.46 mmol) in DMF (6 mL) was added to the mixture under heat. Then the mixture was stirred for 5 minutes and used in the next step.

### Step 2. Preparation of tert-butyl4-((4-(bis(tert-butoxycarbonyl)amino)-1-(2-((tert-butoxycarbonyl)oxy)-2-methylpropyl)-2-(ethoxymethyl)-1H-imidazo[4,5-c]quinolin-7-yl)methyl)piperidine-1-carboxylate

To a solution of tert-butyl(7-bromo-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate (150 mg, 0.215 mmol), PdCl₂(PPh₃)₂(120 mg, 0.17 mmol) and CuI (100 mg, 0.53 mmol) in DMF (3 mL) was added ((1-(tert-butoxycarbonyl)piperidin-4-yl)methyl)zinc(II) iodide (5.5 mL) under N₂. Then the mixture was stirred at 50° C. for 1 hour. TLC (n-Heptane:EA=1:1) showed the reaction was OK. It was poured into saturated NH₄Cl (aq.) (40 mL) and extracted with EA (30 mL*2), the combined organic phases were washed with water (20 mL*2) and dried over anhydrous Na₂SO₄, concentrated on vacuum to give the crude. The residue was purified by silica gel column chromatography (eluting with PE:EA=3:1) to give the product (130 mg, 79.3% yield) as a yellow oil.

LCMS (ESI) calcd for C₄₃H₆₅N₅O₁₀: [M+H]⁺ m/z=812.47, found 812.7.

### Step 3. Preparation of 1-(4-amino-2-(ethoxymethyl)-7-(piperidin-4-ylmethyl)-1H-imidazo[4,5-c]quinolin-1-yl)-2-methylpropan-2-ol

To a solution of tert-butyl4-((4-(bis(tert-butoxycarbonyl)amino)-1-(2-((tert-butoxycarbonyl)oxy)-2-methylpropyl)-2-(ethoxymethyl)-1H-imidazo[4,5-c]quinolin-7-yl)methyl)piperidine-1-carboxylate (130 mg, 0.16 mmol) in DCM (2 mL) was added TFA (1 mL) dropwise at room temperature. The reaction mixture was stirred at room temperature for 3 hours. HPLC showed the reaction was OK. The solution was removed under reduced pressure to afford the crude product. The residue was purified by Prep-HPLC (column: Gemini-C18 250×21.2 mm, 10 um; mobile phase: ACN-H₂O (0.1% TFA); gradient: 20%-65%) to afford Compound 164 (21.9 mg, 33.6% yield) as white solid.

MS (ESI) calcd for C₂₃H₃₃N₅O₂: [M+H]⁺ m/z=412.26, found 412.20.

¹H NMR (400 MHz, DMSO-d6) δ 13.89 (s, 1H), 9.14 (s, 2H), 8.57 (s, 1H), 8.44 (d, J=8.6 Hz, 1H), 8.29 (s, 1H), 7.55 (s, 1H), 7.35 (d, J=8.4 Hz, 1H), 4.72 (s, 6H), 4.19 (s, 7H), 3.55 (q, J=7.0 Hz, 2H), 3.27 (d, J=10.3 Hz, 2H), 2.92-2.77 (m, 2H), 2.72 (d, J=6.9 Hz, 2H), 1.88 (s, 1H), 1.74 (d, J=13.4 Hz, 2H), 1.37 (dd, J=23.4, 11.2 Hz, 2H), 1.16 (dd, J=16.3, 9.3 Hz, 9H).

### Example 58

### Synthesis of Compound 165

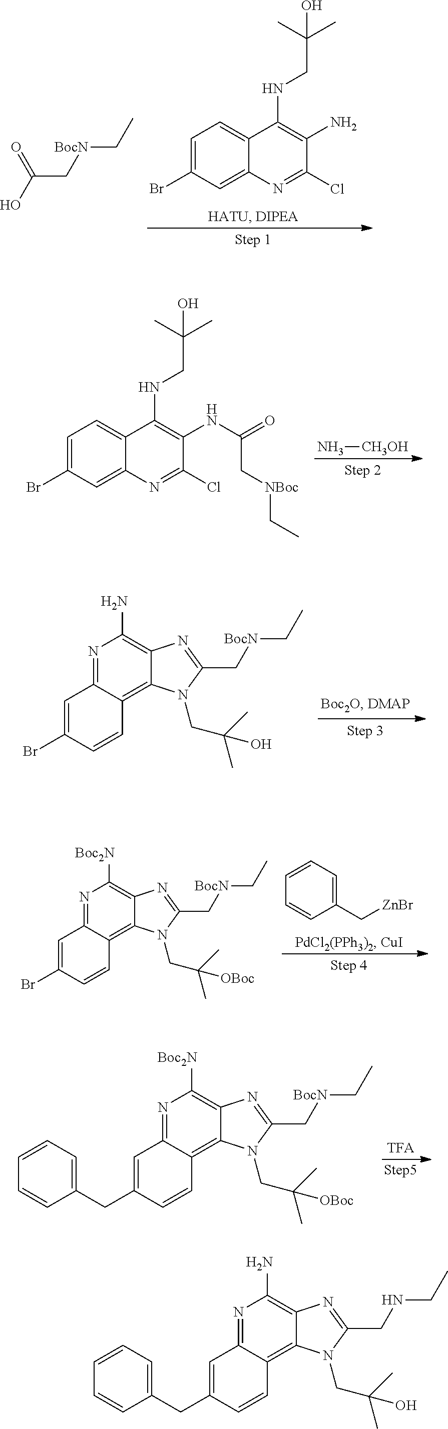

### Step 1. Preparation of tert-butyl (2-((7-bromo-2-chloro-4-((2-hydroxy-2-methylpropyl)amino)quinolin-3-yl)amino)-2-oxoethyl)(ethyl)carbamate

To a solution of N-(tert-butoxycarbonyl)-N-ethylglycine (1.0 g, 4.9 mmol) in DMF (20 mL) was added HATU (2.8 g, 7.4 mmol) and DIPEA (1.9 g, 14.7 mmol) at room temperature. The mixture was stirred at 20° C. for 15 minutes. The mixture was added 1-((3-amino-7-bromo-2-chloroquinolin-4-yl)amino)-2-methylpropan-2-ol (1.7 g, 5.1 mmol). The mixture was stirred at 20° C. for 48 hours. HPLC showed the reaction was completed. The reaction was quenched with saturated NH₄Cl (aq.) (100 mL) and extracted with EA (30 mL*2). The filtrated was concentrated in vacuo to give the crude. The residue was purified by silica gel to give the product (600 mg, 24% yield) as a yellow solid.

LCMS (ESI) calcd for C₂₂H₃₀BrClN₄O₄[M+H]⁺ m/z=529.11, found 529.5.

### Step 2. Preparation of tert-butyl ((4-amino-7-bromo-1-(2-hydroxy-2-methylpropyl)-1H-imidazo[4,5-c]quinolin-2-yl)methyl)(ethyl)carbamate

A mixture of (2-((7-bromo-2-chloro-4-((2-hydroxy-2-methylpropyl)amino)quinolin-3-yl)amino)-2-oxoethyl)(ethyl)carbamate (600 mg, 1.13 mmol) and NH₃—CH₃OH (7M) (15 mL) in closed pot under 160° C. for 8 hours. HPLC showed the reaction was OK and concentrated in vacuo to give the crude. The crude was purified by silica gel column chromatography (eluting with DCM:MeOH=30:1) to give the product (310 mg, 55.9% yield) as a yellow solid.

LCMS (ESI) calcd for C₂₂H₃₀BrN₅O₃[M+H]⁺ m/z=492.15, found 492.2.

### Step 3. Preparation of tert-butyl(7-bromo-2-(((tert-butoxycarbonyl)(ethyl)amino)methyl)-1-(2-((tert-butoxycarbonyl)oxy)-2-methylpropyl)-1H-imidazo[4,5-c]quinolin-4-yl)(tert-butoxycarbonyl)carbamate

To a solution of ((4-amino-7-bromo-1-(2-hydroxy-2-methylpropyl)-1H-imidazo[4,5-c]quinolin-2-yl)methyl)(ethyl)carbamate (310 mg, 0.63 mmol), Boc₂O (1.37 g, 6.3 mmol) and DMAP (40 mg, 0.32 mmol) in DMF (6.2 mL). Then the mixture was stirred at 80° C. for 3 hours. HPLC showed the reaction was OK. The residue was purified by silica gel column chromatography (eluting with PE:EA=3:1) to give the product (360 mg, 72.2% yield) as a yellow solid.

LCMS (ESI) calcd for C₃₇H₅₄BrN₅O₉[M+H]⁺ m/z=792.31, found 792.4.

### Step 4. Preparation of tert-butyl (7-benzyl-2-(((tert-butoxycarbonyl)(ethyl)amino)methyl)-1-(2-((tert-butoxycarbonyl)oxy)-2-methylpropyl)-1H-imidazo[4,5-c]quinolin-4-yl)(tert-butoxycarbonyl)carbamate

To a solution of tert-butyl(7-bromo-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate (150 mg, 0.215 mmol), PdCl₂(PPh₃)₂(120 mg, 0.17 mmol) and CuI (100 mg, 0.53 mmol) in DMF (3 mL) was added benzylzinc(II) bromide (5.5 mL) under N₂. Then the mixture was stirred at 50° C. for 1 hour. LCMS showed the reaction was OK. It was poured into saturated NH₄Cl (aq.) (40 mL) and extracted with EA (30 mL*2), the combined organic phases were washed with water (20 mL*2) and dried over anhydrous Na₂SO₄, concentrated in vacuo to give the crude. The residue was purified by silica gel column chromatography (eluting with PE:EA=3:1) to give the product (183 mg, 83.3% yield) as a yellow oil.

LCMS (ESI) calcd for C₄₄H₆₁N₅O₉[M+H]⁺ m/z=804.45, found 804.7.

### Step 5. Preparation of 1-(4-amino-7-benzyl-2-((ethylamino)methyl)-1H-imidazo[4,5-c]quinolin-1-yl)-2-methylpropan-2-ol

To a solution of (7-benzyl-2-(((tert-butoxycarbonyl)(ethyl)amino)methyl)-1-(2-((tert-butoxycarbonyl)oxy)-2-methylpropyl)-1H-imidazo[4,5-c]quinolin-4-yl)(tert-butoxycarbonyl)carbamate (183 mg, 0.23 mmol) in DCM (2 mL) was added TFA (1 mL) dropwise at room temperature. The reaction mixture was stirred at room temperature for 3 hours. HPLC showed the reaction was OK. The solution was removed under reduced pressure to afford the crude product. The residue was purified by Prep-HPLC (column: Gemini-C18 250×21.2 mm, 10 um; mobile phase: ACN-H₂O (0.1% TFA); gradient: 20%-65%) to afford Compound 165 (86 mg, 23.6% yield) as white solid.

MS (ESI) calcd for C₂₄H₂₉N₅O [M+H]⁺ m/z=404.24, found 404.0.

¹H NMR (400 MHz, DMSO-d6) δ 14.00 (s, 1H), 9.27 (s, 2H), 8.95 (s, 2H), 8.45 (d, J=8.6 Hz, 1H), 7.61 (s, 1H), 7.41 (d, J=8.5 Hz, 1H), 7.37-7.28 (m, 4H), 7.24 (t, J=6.9 Hz, 1H), 5.23 (s, 1H), 4.70 (s, 4H), 4.15 (s, 2H), 3.23 (d, J=6.6 Hz, 2H), 1.34-1.00 (m, 9H).

### Example 59

### Synthesis of Compound 166

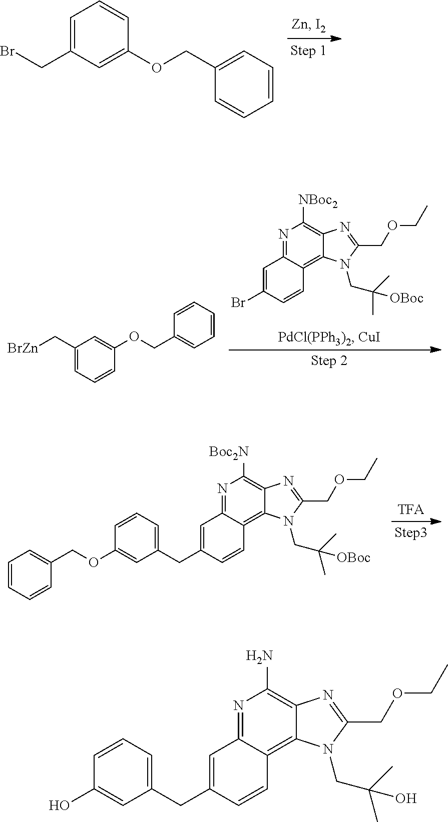

### Step 1. Preparation of (3-(benzyloxy)benzyl)zinc(II) bromide

A mixture of Zn (1.75 g, 26.8 mmol) and 12 (0.15 g, 0.59 mmol) in N₂ protection was stirred 5 minutes under heat. Then 1-(benzyloxy)-3-(bromomethyl)benzene (1.24 g, 4.46 mmol) in DMF (6 mL) was added to the mixture under heat. Then the mixture was stirred for 5 minutes and used in the next step.

### Step 2. Preparation of tert-butyl(7-(3-(benzyloxy)benzyl)-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate

To a solution of tert-butyl(7-bromo-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate (300 mg, 0.433 mmol), PdCl₂(PPh₃)₂(120 mg, 0.17 mmol) and CuI (100 mg, 0.53 mmol) in DMF (3 mL) was added (3-(benzyloxy)benzyl)zinc(II) bromide (5.5 mL) under N₂. Then the mixture was stirred at 50° C. for 1 hour. TLC (n-Heptane:EA=1:1) showed the reaction was OK. It was poured into saturated NH₄Cl (aq) (40 mL) and extracted with EA (30 mL*2), the combined organic phases were washed with water (20 mL*2) and dried over anhydrous Na₂SO₄, concentrated on vacuum to give the crude. The residue was purified by silica gel column chromatography (eluting with PE:EA=3:1) to give the product (258 mg, 86.8% yield) as a yellow oil.

LCMS (ESI) calcd for C₄₆H₅₈N₄O₉ [M+H]⁺ m/z=811.42, found 811.7.

### Step 3. Preparation of 3-((4-amino-2-(ethoxymethyl)-1-(2-hydroxy-2-methylpropyl)-1H-imidazo[4,5-c]quinolin-7-yl)methyl)phenol

To a solution of tert-butyl(7-(3-(benzyloxy)benzyl)-1-(2-((tert-butoxycarbonyl)oxy)-2-methylpropyl)-2-(ethoxymethyl)-1H-imidazo[4,5-c]quinolin-4-yl)(tert-butoxycarbonyl)carbamate (130 mg, 0.41 mmol) was added TFA (1 mL). The reaction mixture was stirred at 50° C. for 1 hour. HPLC showed the reaction was OK. The residue was purified by Prep-HPLC (column: Gemini-C18 250×21.2 mm, 10 um; mobile phase: ACN-H₂O (0.1% TFA); gradient: 20%-65%) to afford Compound 166 (19.5 mg, 29.1% yield) as white solid.

MS (ESI) calcd for C₂₄H₂₈N₄O₃ [M+H]⁺ m/z=421.22, found 421.14.

¹H NMR (400 MHz, DMSO-d6) δ 13.27 (s, 1H), 9.36 (s, 1H), 8.42 (d, J=8.7 Hz, 1H), 7.55 (s, 1H), 7.38 (d, J=8.7 Hz, 1H), 7.12 (t, J=7.7 Hz, 1H), 6.71 (d, J=7.4 Hz, 1H), 6.68-6.60 (m, 2H), 4.93 (s, 2H), 4.70 (s, 2H), 4.03 (s, 2H), 3.54 (q, J=6.9 Hz, 2H), 1.16 (dd, J=25.8, 18.8 Hz, 9H).

### Example 60

### Synthesis of Compound 168

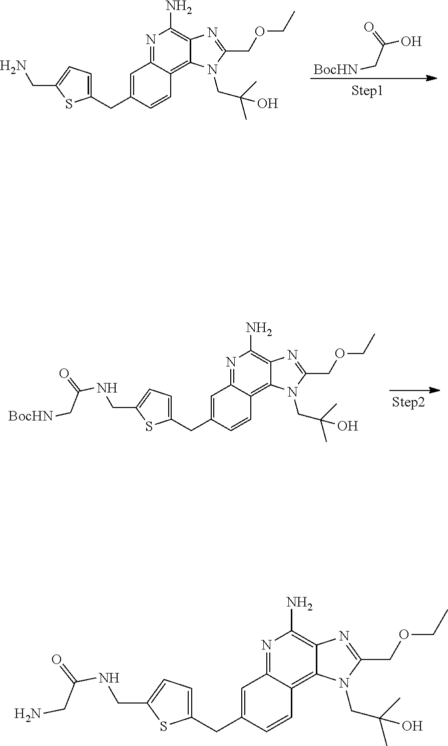 [SMILES: CCOCc1nc2c(N)nc3cc(Cc4ccc(CN)s4)ccc3c2n1CC(C)(C)O ; CC(C)(C)OC(=O)NCC(=O)O ; CCOCc1nc2c(N)nc3cc(Cc4ccc(CNC(=O)CNC(=O)OC(C)(C)C)s4)ccc3c2n1CC(C)(C)O ; CCOCc1nc2c(N)nc3cc(Cc4ccc(CNC(=O)CN)s4)ccc3c2n1CC(C)(C)O]

### Step 1. Preparation of tert-butyl (2-(((5-((4-amino-2-(ethoxymethyl)-1-(2-hydroxy-2-methylpropyl)-1H-imidazo[4,5-c]quinolin-7-yl)methyl)thiophen-2-yl)methyl)amino)-2-oxoethyl)carbamate

To a solution of (tert-butoxycarbonyl)glycine (15.0 mg, 0.0857 mmol) in DMF (1 mL) was added HOSU (11.8 mg, 0.1027 mmol) and EDCI (32.8 mg, 0.1714 mmol) and the mixture was stirred at room temperature for 2 hours. 1-(4-Amino-7-((5-(aminomethyl)thiophen-2-yl)methyl)-2-(ethoxymethyl)-1H-imidazo[4,5-c]quinolin-1-yl)-2-methylpropan-2-ol (18.8 mg, 0.0428 mmol) and DIEA (33.2 mg, 0.2571 mmol) was added to the mixture and stirred for another 1 hours. HPLC showed the reaction was completed. The reaction mixture was purified by Prep-HPLC (Column: Gemini-C18, 250×50 mm, 10 um; Mobile Phase: ACN-H₂O (0.1% TFA), Gradient: 10%-65%) to give the product (15.0 mg, 29.3% yield) as a white solid.

### Step 2. Preparation of 2-amino-N-((5-((4-amino-2-(ethoxymethyl)-1-(2-hydroxy-2-methylpropyl)-1H-imidazo[4,5-c]quinolin-7-yl)methyl)thiophen-2-yl)methyl)acetamide

To a solution of tert-butyl (2-(((5-((4-amino-2-(ethoxymethyl)-1-(2-hydroxy-2-methylpropyl)-1H-imidazo[4,5-c]quinolin-7-yl)methyl)thiophen-2-yl)methyl)amino)-2-oxoethyl)carbamate (15 mg, 0.0251 mmol) in DCM (1 mL) was added TFA (0.3 mL) drop wise at room temperature. The reaction mixture was stirred at room temperature for 1 hour. HPLC showed the reaction was OK. The reaction mixture was purified by Prep-HPLC (Column: Gemini-C18, 250×50 mm, 10 um; Mobile Phase: ACN-H₂O (0.1% TFA), Gradient: 10%-65%) to give the Compound 168 (8.1 mg, 64.7% yield) as a white solid.

MS (ESI) calcd for C₅₆H₆₅N₁₁O₁₁S [M+H]⁺ m/z=497.23, found 497.2.

### Example 61

### Synthesis of Compound 169

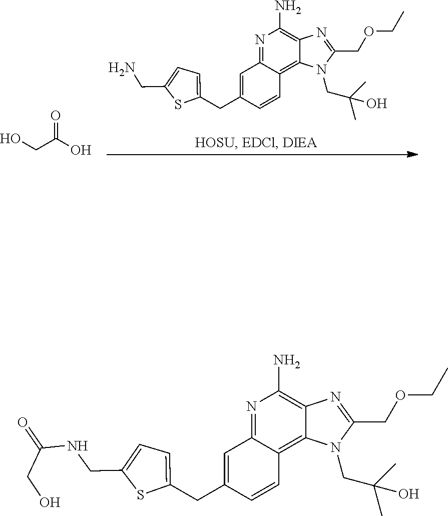 [SMILES: O=C(O)CO ; CCOCc1nc2c(N)nc3cc(Cc4ccc(CN)s4)ccc3c2n1CC(C)(C)O ; CCOCc1nc2c(N)nc3cc(Cc4ccc(CNC(=O)CO)s4)ccc3c2n1CC(C)(C)O]

To a solution of 2-hydroxyacetic acid (5.19 mg, 0.0683 mmol) in DMF (1 mL) was added HOSU (7.85 mg, 0.0483 mmol) and EDCI (13.05 mg, 0.0683 mmol) and the mixture was stirred at room temperature for 2 hours. 1-(4-amino-7-((5-(aminomethyl)thiophen-2-yl)methyl)-2-(ethoxymethyl)-1H-imidazo[4,5-c]quinolin-1-yl)-2-methylpropan-2-ol (20 mg, 0.0455 mmol) and DIEA (15.2 mg, 0.1176 mmol) was added to the mixture and stirred for another 1 hour. HPLC showed the reaction was completed. The reaction mixture was purified by Prep-HPLC (Column: Gemini-C18, 250×50 mm, 10 um; Mobile Phase: ACN-H₂O (0.1% TFA), Gradient: 10%-65%) to give the Compound 169 (11.2 mg, 49.46% yield) as a white solid.

MS (ESI) calcd for C₂₅H₃₁N₅O₄S [M+H]⁺ m/z=498.21, found 498.2.

### Example 62

### Synthesis of Compound 171

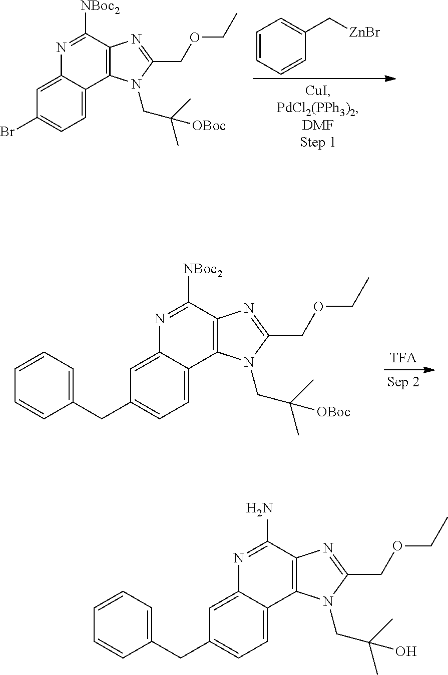

### Step 1: Preparation of di-tert-butyl (7-benzyl)-1-(2-((tert-butoxycarbonyl)oxy)-2-methylpropyl)-2-(ethoxymethyl)-1H-imidazo[4,5-c]quinolin-4-yl)iminodicarbonate

To a solution of di-tert-butyl (7-bromo-1-(2-((tert-butoxycarbonyl) oxy)-2-methylpropyl)-2-(ethoxymethyl)-1H-imidazo[4,5-c]quinolin-4-yl) iminodicarbonate (693 mg, 1 mmol), Pd(PPh₃)Cl₂ (81 mg, 0.12 mmol) and CuI (43 mg, 0.23 mmol) in DMF (10 mL) was added a solution of benzyl zinc(II) bromide in DMF (15 mL). The mixture was stirred at 50′C for 1 h. LCMS showed the reaction was completed. The resulting mixture was diluted with water (60 mL) and extracted with EA (40 mL×3). The combine organic phases were washed with brine, dried over Na₂SO₄, concentrated to give the crude. The residue was purified by silica gel column chromatography (PE:EA=1:1) to afford the product (423 mg, 70% yield) as yellow solid.

### Step 2: Preparation of 1-(4-amino-7-benzyl-2-(ethoxymethyl)-1H-imidazo[4,5-c]quinolin-1-yl)-2-methylpropan-2-ol

To a solution of di-tert-butyl (7-benzyl)-1-(2-((tert-butoxycarbonyl)oxy)-2-methylpropyl)-2-(ethoxymethyl)-1H-imidazo[4,5-c]quinolin-4-yl)iminodicarbonate (350 mg, 0.5 mmol) in DCM (6 mL) was added TFA (2 mL). The mixture was stirred at RT for 4 hours. LCMS showed the reaction was completed. The resulting mixture was adjusted to pH=8 with aqueous NaHCO₃ and extracted with EA (50 mL×3). The combined organic phases were washed with water and brine, dried over sodium sulfate, concentrated under vacuum to give the crude. The crude was purified by Prep-HPLC (Column: Gemini-C18, 150×21.2 mm, 5 um; Mobile Phase: ACN-H₂O (0.10% FA), Gradient: 10-40) to give the Compound 171 (344 mg, 85% yield) as a white solid.

### Example 63

### General Synthesis of Linker-Payload (Mc-VC-PAB-Payload)

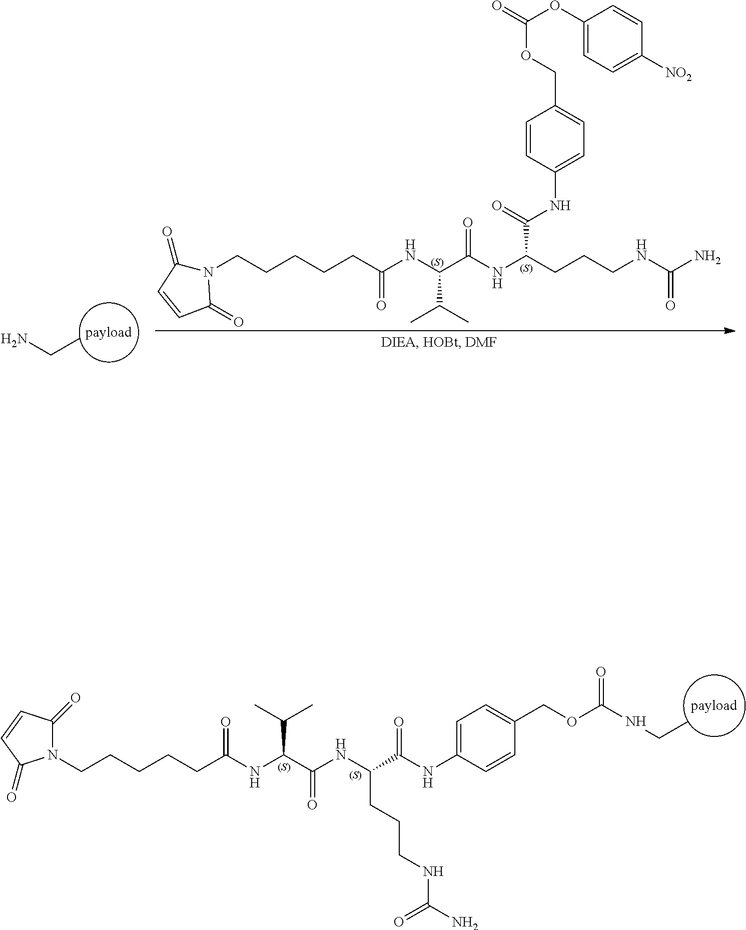

To a solution of a payload (0.114 mmol) and 4-((S)-2-((S)-2-(6-(2,5-dioxo-2,5-dihydro-1H-pyrrol-1-yl)hexanamido)-3-methylbutanamido)-5-ureidopentanamido)benzyl (4-nitrophenyl) carbonate (0.228 mmol) in DMF (0.5 mL) was added DIPEA (22 mg, 0.171 mmol) dropwise. Then stirred at 20° C. for 1.5 h. HPLC and LCMS showed the reaction was OK. Added MTBE (50 mL) into the reaction precipitated the crude, filtered and concentrated in vacuum. The residue was purified by Prep-HPLC (Column: Gemini-C18, 250×21.2 mm, 10 um; Mobile Phase: ACN-H₂O (0.1% TFA), Gradient: 20%-65%) to afford linker-payload (Mc-VC-PAB-payload).

### Example 64

### Synthesis of Linker-Payload (Mc-VC-PAB-Compound 165)

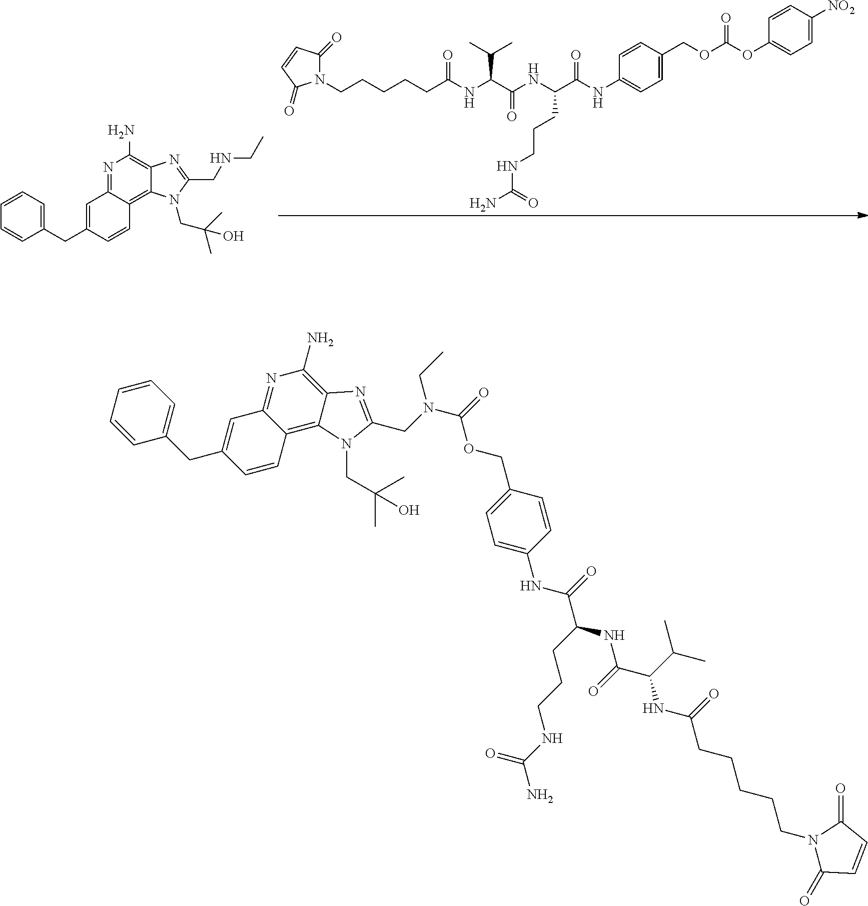 [SMILES: CCNCc1nc2c(N)nc3cc(Cc4ccccc4)ccc3c2n1CC(C)(C)O ; CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1 ; CCN(Cc1nc2c(N)nc3cc(Cc4ccccc4)ccc3c2n1CC(C)(C)O)C(=O)OCc1ccc(NC(=O)[C@H](CCCNC(N)=O)NC(=O)[C@@H](NC(=O)CCCCCN2C(=O)C=CC2=O)C(C)C)cc1]

### Preparation of 4-((S)-2-((S)-2-(6-(2,5-dioxo-2,5-dihydro-1H-pyrrol-1-yl)hexanamido)-3-methylbutanamido)-5-ureidopentanamido)benzyl ((4-amino-7-benzyl-1-(2-hydroxy-2-methylpropyl)-1H-imidazo[4,5-c]quinolin-2-yl)methyl) (ethyl)carbamate

To a solution Compound 165 (28 mg, 0.069 mmol) and 4-((S)-2-((S)-2-(6-(2,5-dioxo-2,5-dihydro-1H-pyrrol-1-yl)hexanamido)-3-methylbutanamido)-5-ureidopentanamido)benzyl (4-nitrophenyl) carbonate (56 mg, 0.076 mmol) in DMF (0.5 mL) was added DIPEA (13.5 mg, 0.104 mmol) dropwise. Then stirred at room temperature for 30 minutes. HPLC showed the reaction was OK. The residue was purified by Prep-HPLC (Column: Gemini-C18, 250×21.2 mm, 10 um; Mobile Phase: ACN-H₂O (0.1% TFA), Gradient: 20%-65%) to afford the linker-payload (Mc-VC-PAB-Compound 165) (32 mg, 46.3% yield) as white solid.

MS (ESI) calcd for C₅₃H₆₇N₁₁O₉ [M+H]⁺ m/z=1002.01, found 1002.45.

### Example 65

### Synthesis of Linker-payload (Mc-glucuronide-Compound 171)

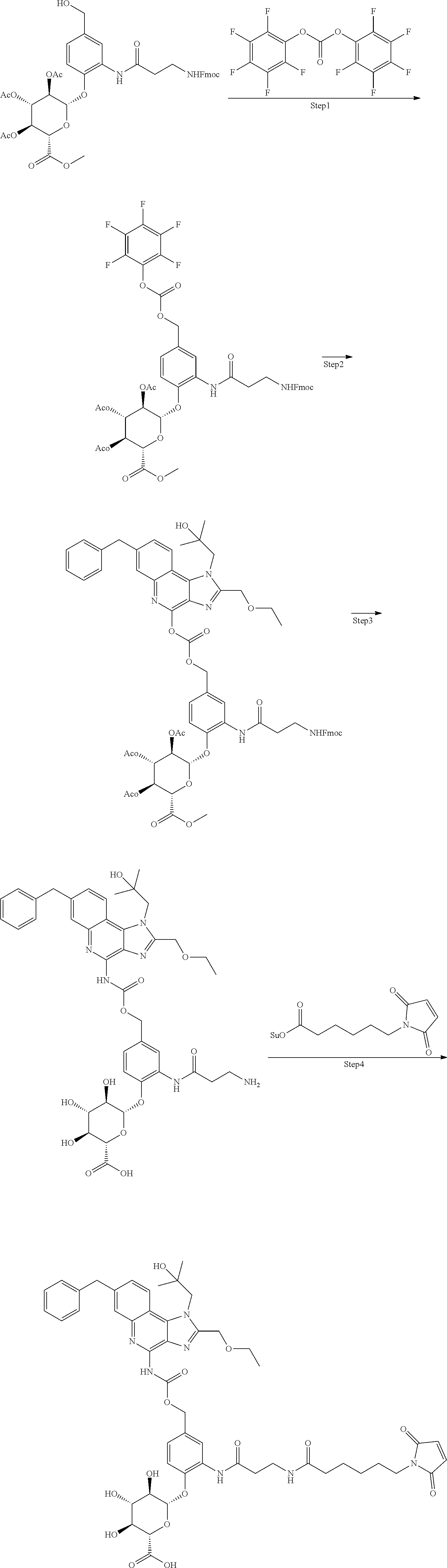

### Step 1. Preparation of (2S,3R,4S,5S,6S)-2-(2-(3-((((9H-fluoren-9-yl) methoxy) carbonyl)amino)propanamido)-4-((((perfluorophenoxy)carbonyl)oxy)methyl) phenoxy)-6-(methoxycarbonyl)tetrahydro-2H-pyran-3,4,5-triyl triacetate

To a solution of (2S,3R,4S,5S,6S)-2-(2-(3-((((9H-fluoren-9-yl) methoxy) carbonyl) amino) propanamido)-4-(hydroxymethyl)phenoxy)-6-(methoxycarbonyl)tetrahydro-2H-pyran-3,4,5-triyl triacetate (500 mg, 0.6677 mmol) in DCM (5 mL) was added bis(perfluorophenyl) carbonate (315.8 mg, 0.8012 mmol) and DIEA (198.5 g, 1.5357 mmol) at room temperature. The mixture was stirred at room temperature for 16 hours. TLC (n-Heptane:EA=1:2) showed the reaction was OK. The mixture was diluted with H₂O (80 mL) and extracted with DCM (100 mL*2), the combined organic were washed with saturated NaCl (aq.) (100 mL*2) and dried over anhydrous Na₂SO₄, concentrated in vacuo to give the crude. The residue was purified by silica gel column chromatography (eluting with PE:EA=1:1) to give the title compound (432 mg, 67% yield) as a white solid.

MS (ESI) calcd for C₄₅H₃₉F₅N₂O₁₆ [M+H]⁺ m/z=959.22, found 959.1.

### Step 2. Preparation of (2S,3R,4S,5S,6S)-2-(2-(3-((((9H-fluoren-9-yl)methoxy)carbonyl)amino)propanamido)-4-((((7-benzyl-2-(ethoxymethyl)-1-(2-hydroxy-2-methylpropyl)-1H-imidazo[4,5-c]quinolin-4-yl)carbamoyl)oxy)methyl)phenoxy)-6-(methoxycarbonyl)tetrahydro-2H-pyran-3,4,5-triyl triacetate

To a solution of (2S,3R,4S,5S,6S)-2-(2-(3-((((9H-fluoren-9-yl)methoxy)carbonyl)amino)propanamido)-4-((((perfluorophenoxy)carbonyl)oxy)methyl)phenoxy)-6-(methoxycarbonyl)tetrahydro-2H-pyran-3,4,5-triyl triacetate (432 mg, 0.4533 mmol) in DMF (3 mL) was added Compound 171 (95 mg, 0.3022 mmol), HOAt (20.5 mg, 0.1511 mmol) and DIEA (117 mg, 0.9066 mmol) at room temperature. The mixture was stirred at 30° C. for 16 hours. HPLC showed the reaction was OK. The mixture was purified by Prep-HPLC (column: Gemini-C18 250*21.2 mm, 10 um; mobile phase: ACN-H₂O (0.10% TFA); gradient: 20%-65%) to afford the title compound (130 mg, 39.4% yield) as a light yellow solid.

MS (ESI) calcd for C₅₃H₆₇N₆O₁₇ [M+H]⁺ m/z=1179.46, found 1179.3.

### Step 3. Preparation of (2S,3S,4S,5R,6S)-6-(2-(3-aminopropanamido)-4-((((7-benzyl-2-(ethoxymethyl)-1-(2-hydroxy-2-methylpropyl)-1H-imidazo[4,5-c]quinolin-4-yl)carbamoyl)oxy)methyl)phenoxy)-3,4,5-trihydroxytetrahydro-2H-pyran-2-carboxylic acid

To a solution of (2S,3R,4S,5S,6S)-2-(2-(3-((((9H-fluoren-9-yl)methoxy)carbonyl)amino)propanamido)-4-((((perfluorophenoxy)carbonyl)oxy)methyl)phenoxy)-6-(methoxycarbonyl)tetrahydro-2H-pyran-3,4,5-triyl triacetate (51 mg, 0.0459 mmol) in DCM (1 mL) was added Et₂NH (0.2 mL) under N₂. The mixture was stirred at room temperature for 6 hours. HPLC showed the reaction was OK. Then reaction mixture was Concentrated to dryness. Then the mixture was added THF (1 mL) and 0.2 N LiOH (1 mL) at 0° C. The mixture was stirred at 0° C. for 1 hour. HPLC showed the reaction was OK. It was poured into saturated NH₄Cl (aq.) (40 mL) and concentrated THF in vacuum. The mixture was purified by Prep-HPLC (column: Gemini-C18 250*21.2 mm, 10 um; mobile phase: ACN-H₂O (0.1% TFA); gradient: 20%-65%) to afford product (10 mg, 30% yield) as a yellow solid.

### Step 4. Preparation of (2S,3S,4S,5R,6S)-6-(4-((((7-benzyl-2-(ethoxymethyl)-1-(2-hydroxy-2-methylpropyl)-1H-imidazo[4,5-c]quinolin-4-yl)carbamoyl)oxy)methyl)-2-(3-(6-(2,5-dioxo-2,5-dihydro-1H-pyrrol-1-yl)hexanamido)propanamido)phenoxy)-3,4,5-trihydroxytetrahydro-2H-pyran-2-carboxylic acid

To a solution of (2S,3S,4S,5R,6S)-6-(2-(3-aminopropanamido)-4-((((7-benzyl-2-(ethoxymethyl)-1-(2-hydroxy-2-methylpropyl)-1H-imidazo[4,5-c]quinolin-4-yl)carbamoyl)oxy)methyl)phenoxy)-3,4,5-trihydroxytetrahydro-2H-pyran-2-carboxylic acid (10 mg, 0.0.0137 mmol) in DMA (0.5 mL) was added 2,5-dioxopyrrolidin-1-yl 6-(2,5-dioxo-2,5-dihydro-1H-pyrrol-1-yl)hexanoate (6.4 mg, 0.0206 mmol) and DIEA (7.1 mg, 0.0548 mmol) at room temperature. The mixture was stirred at room temperature for 1 hour. HPLC showed the reaction was OK. The mixture was purified by Prep-HPLC (column: Gemini-C18 250*21.2 mm, 10 um; mobile phase: ACN-H₂O (0.10% TFA); gradient: 20%-65%) to afford product (8.5 mg, 67% yield) as a white solid.

LCMS (ESI) calcd for C₅₁H₅₈N₇O₁₄ [M−18+H]⁺ m/z=992.40, found 992.3.

### Example 66

### Synthesis of Linker-Payload (Mc-GGFG-Compound 147)

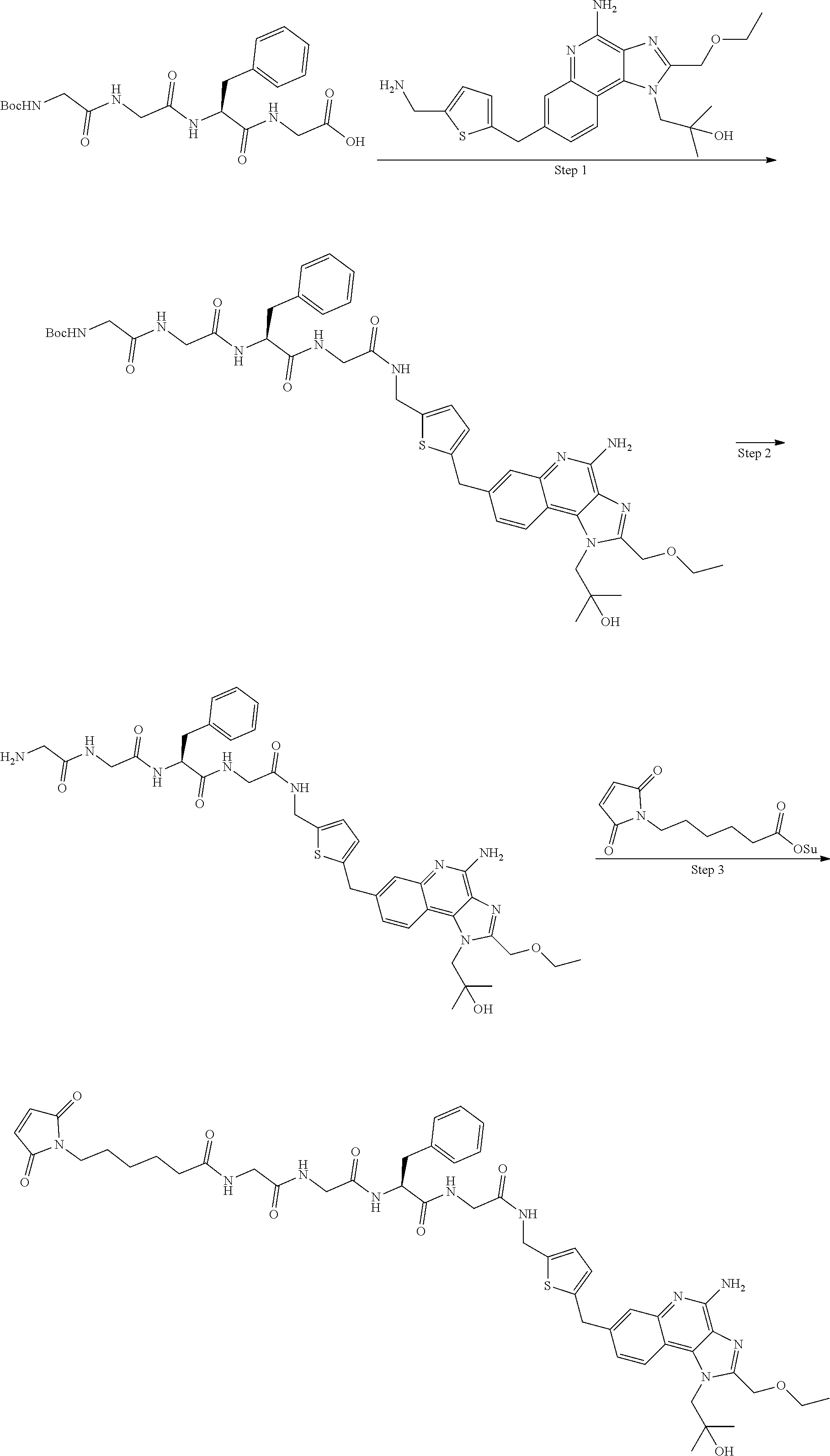

### Step 1. Preparation of tert-butyl (S)-(1-(5-((4-amino-2-(ethoxymethyl)-1-(2-hydroxy-2-methylpropyl)-1H-imidazo[4,5-c]quinolin-7-yl)methyl)thiophen-2-yl)-7-benzyl-3,6,9,12-tetraoxo-2,5,8,11-tetraazatridecan-13-yl)carbamate

To a solution of (tert-butoxycarbonyl)glycylglycyl-L-phenylalanylglycine (70 mg, 0.160 mmol) in DMF (5 mL) was added HATU (91.5 mg, 0.241 mmol) and 1-(4-amino-7-((5-(aminomethyl)thiophen-2-yl)methyl)-2-(ethoxymethyl)-1H-imidazo[4,5-c]quinolin-1-yl)-2-methylpropan-2-ol (70.24 mg, 0.160 mmol), DIEA (61.92 mg, 0.479 mmol) and the mixture was stirred at room temperature for 3 hours. HPLC showed the reaction was completed. The reaction mixture was purified by Prep-HPLC (Column: Gemini-C18, 250×50 mm, 10 um; Mobile Phase: ACN-H₂O (0.1% TFA), Gradient: 10%-65%) to give the product (65 mg, 47.2% yield) as a white solid.

LCMS (ESI) calcd for C₄₃H₅₅N₉O₈S [M+H]⁺ m/z=858.39, found 858.4.

### Step 2. Preparation of (S)—N-(2-(((5-((4-amino-2-(ethoxymethyl)-1-(2-hydroxy-2-methylpropyl)-1H-imidazo[4,5-c]quinolin-7-yl)methyl)thiophen-2-yl)methyl)amino)-2-oxoethyl)-2-(2-(2-aminoacetamido)acetamido)-3-phenylpropanamide

To a solution of tert-butyl (S)-(1-(5-((4-amino-2-(ethoxymethyl)-1-(2-hydroxy-2-methylpropyl)-1H-imidazo[4,5-c]quinolin-7-yl)methyl)thiophen-2-yl)-7-benzyl-3,6,9,12-tetraoxo-2,5,8,11-tetraazatridecan-13-yl)carbamate (65 mg, 0.076 mmol) in DCM (2 mL) was added TFA (1 mL) drop wise at room temperature. The reaction mixture was stirred at room temperature for 3 hours. HPLC showed the reaction was OK. The solution was removed under reduced pressure to afford the crude product (65 mg, 100% yield) as yellow oil.

### Step 3. Preparation of (S)—N-(1-(5-((4-amino-2-(ethoxymethyl)-1-(2-hydroxy-2-methylpropyl)-1H-imidazo[4,5-c]quinolin-7-yl)methyl)thiophen-2-yl)-7-benzyl-3,6,9,12-tetraoxo-2,5,8,11-tetraazatridecan-13-yl)-6-(2,5-dioxo-2,5-dihydro-1H-pyrrol-1-yl)hexanamide

To a solution of (S)—N-(2-(((5-((4-amino-2-(ethoxymethyl)-1-(2-hydroxy-2-methylpropyl)-1H-imidazo[4,5-c]quinolin-7-yl)methyl)thiophen-2-yl)methyl)amino)-2-oxoethyl)-2-(2-(2-aminoacetamido)acetamido)-3-phenylpropanamide (65 mg, 0.076 mmol) and 2,5-dioxopyrrolidin-1-yl 6-(2,5-dioxo-2,5-dihydro-1H-pyrrol-1-yl)hexanoate (35 mg, 0.114 mmol) in DMF (3 mL) was added DIEA (29.42 mg, 0.228 mmol) and the mixture was stirred at room temperature for 1 hour. HPLC showed the reaction was OK. The reaction mixture was purified by Prep-HPLC (Column: Gemini-C18, 250×50 mm, 10 um; Mobile Phase: ACN-H₂O (0.1% TFA), Gradient: 10%-65%) to give the product (39.5 mg, 96.04% purity, 214 nm, 48.3% yield) as a white solid.

MS (ESI) calcd for C₄₈H₅₈N₁₀O₉S [M+H]⁺ m/z=951.41, found 95 1.3.

### Example 67

### Synthesis of Linker-Payload (Mc-GGFG-PAB-Compound 147)

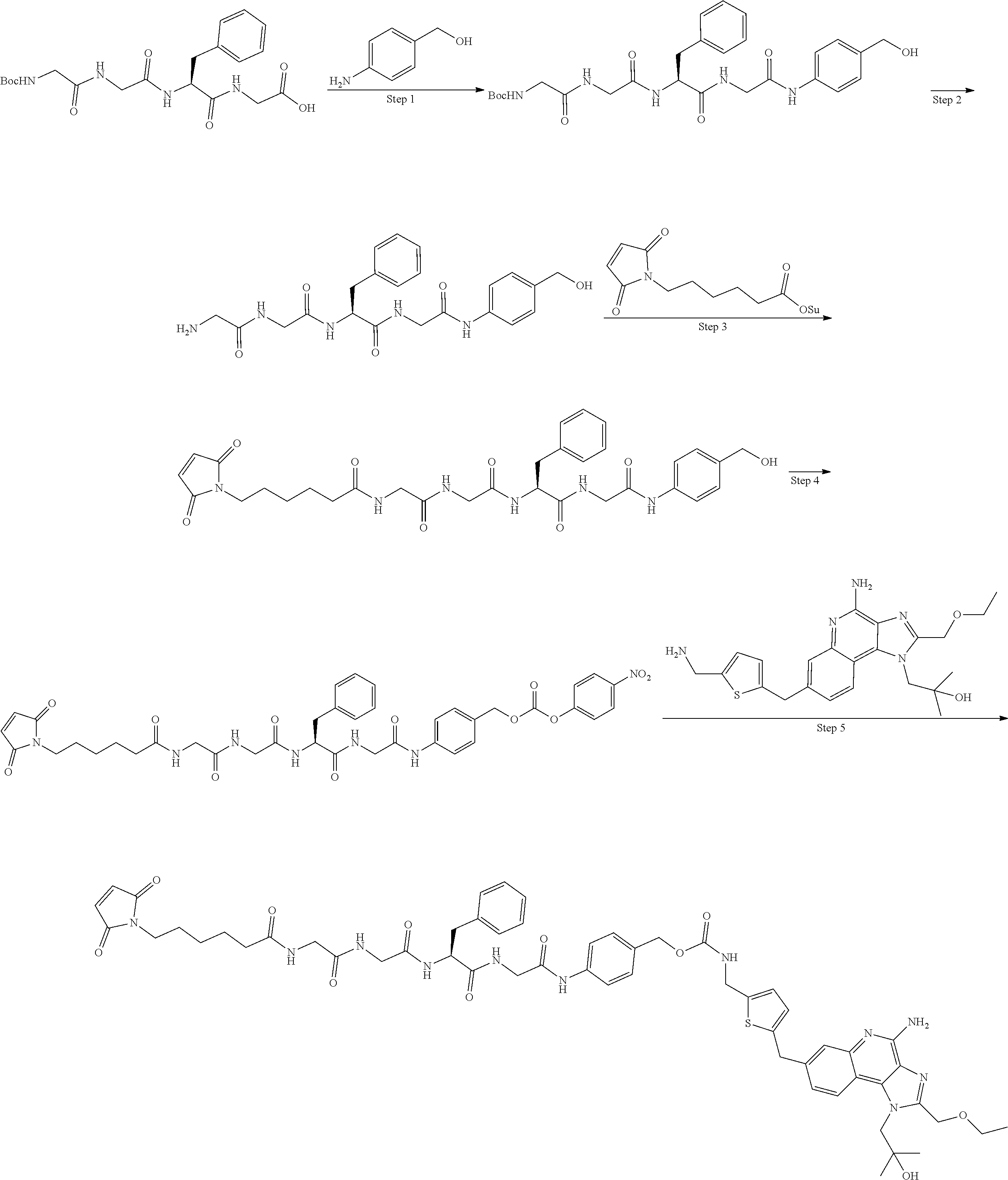

### Step 1. Preparation of tert-butyl (S)-(2-((2-((1-((2-((4-(hydroxymethyl)phenyl)amino)-2-oxoethyl)amino)-1-oxo-3-phenylpropan-2-yl)amino)-2-oxoethyl)amino)-2-oxoethyl)carbamate

To a solution of (tert-butoxycarbonyl)glycylglycyl-L-phenylalanylglycine (500 mg, 1.15 mmol) in DMF (5 mL) was added HATU (655.5 mg, 1.725 mmol) and (4-aminophenyl)methanol (155.16 mg, 1.26 mmol), DIEA (445.05 mg, 1.45 mmol) and the mixture was stirred at room temperature for 3 hours. HPLC showed the reaction was completed. The reaction mixture was purified by Prep-HPLC (Column: Gemini-C18, 250×50 mm, 10 um; Mobile Phase: ACN-H₂O (0.1% TFA), Gradient: 10%-65%) to give the product (600 mg, 96.7% yield) as a white solid.

### Step 2. Preparation of (S)-2-(2-(2-aminoacetamido)acetamido)-N-(2-((4-(hydroxymethyl)phenyl)amino)-2-oxoethyl)-3-phenylpropanamide

To a solution of tert-butyl (S)-(2-((2-((1-((2-((4-(hydroxymethyl)phenyl)amino)-2-oxoethyl)amino)-1-oxo-3-phenylpropan-2-yl)amino)-2-oxoethyl)amino)-2-oxoethyl)carbamate (600 mg, 1.1 mmol) in DCM (5 mL) was added TFA (2 mL) drop wise at room temperature. The reaction mixture was stirred at room temperature for 3 hours. HPLC showed the reaction was OK. The solution was removed under reduced pressure to afford the crude product (450 mg, 92% yield) as yellow oil.

LCMS (ESI) calcd for C₂₂H₂₇N₅O₅ [M+H]⁺ m/z=442.20, found 442.2.

### Step 3. Preparation of (S)-6-(2,5-dioxo-2,5-dihydro-1H-pyrrol-1-yl)-N-(2-((2-((1-((2-((4-(hydroxymethyl)phenyl)amino)-2-oxoethyl)amino)-1-oxo-3-phenylpropan-2-yl)amino)-2-oxoethyl)amino)-2-oxoethyl)hexanamide

To a solution of (S)-2-(2-(2-aminoacetamido)acetamido)-N-(2-((4-(hydroxymethyl)phenyl)amino)-2-oxoethyl)-3-phenylpropanamide (450 mg, 1.15 mmol) and 2,5-dioxopyrrolidin-1-yl 6-(2,5-dioxo-2,5-dihydro-1H-pyrrol-1-yl)hexanoate (353 mg, 1.15 mmol) in DMF (3 mL) was added DIEA (445 mg, 3.45 mmol) and the mixture was stirred at room temperature for 1 hour. HPLC showed the reaction was OK. The reaction mixture was purified by Prep-HPLC (Column: Gemini-C18, 250×50 mm, 10 um; Mobile Phase: ACN-H₂O (0.1% TFA), Gradient: 10%-65%) to give the product (120 mg, 18.54% yield) as a white solid.

LCMS (ESI) calcd for C₃₂H_{3M}N₆O₈ [M−H]⁻ m/z=633.28, found 633.4.

### Step 4. Preparation of (S)-4-(5-benzyl-18-(2,5-dioxo-2,5-dihydro-1H-pyrrol-1-yl)-4,7,10,13-tetraoxo-3,6,9,12-tetraazaoctadecanamido)benzyl (4-nitrophenyl) carbonate

To a solution of (S)-6-(2,5-dioxo-2,5-dihydro-1H-pyrrol-1-yl)-N-(2-((2-((1-((2-((4-(hydroxymethyl)phenyl)amino)-2-oxoethyl)amino)-1-oxo-3-phenylpropan-2-yl)amino)-2-oxoethyl)amino)-2-oxoethyl)hexanamide (120 mg, 0.189 mmol) and bis(4-nitrophenyl) carbonate (90.2 mg, 0.284 mmol) in DMF (3 mL) was added DIEA (73.14 mg, 0.567 mmol) and the mixture was stirred at room temperature for 1 hour. HPLC showed the reaction was OK. The reaction mixture was purified by Prep-HPLC (Column: Gemini-C18, 250×50 mm, 10 um; Mobile Phase: ACN-H₂O (0.1% TFA), Gradient: 10%-65%) to give the product (120 mg, 100% yield) as a white solid.

### Step 5. Preparation of (S)-4-(5-benzyl-18-(2,5-dioxo-2,5-dihydro-1H-pyrrol-1-yl)-4,7,10,13-tetraoxo-3,6,9,12-tetraazaoctadecanamido)benzyl ((5-((4-amino-2-(ethoxymethyl)-1-(2-hydroxy-2-methylpropyl)-1H-imidazo[4,5-c]quinolin-7-yl)methyl)thiophen-2-yl)methyl)carbamate

To a solution of (S)-4-(5-benzyl-18-(2,5-dioxo-2,5-dihydro-1H-pyrrol-1-yl)-4,7,10,13-tetraoxo-3,6,9,12-tetraazaoctadecanamido)benzyl (4-nitrophenyl) carbonate (76.5 mg, 0.0956 mmol) and 1-(4-amino-7-((5-(aminomethyl)thiophen-2-yl)methyl)-2-(ethoxymethyl)-1H-imidazo [4,5-c]quinolin-1-yl)-2-methylpropan-2-ol (28 mg, 0.0638 mmol) in DMF (3 mL) was added DIEA (24.69 mg, 0.191 mmol) and the mixture was stirred at room temperature for 1 hour. HPLC showed the reaction was OK. The reaction mixture was purified by Prep-HPLC (Column: Gemini-C18, 250×50 mm, 10 um; Mobile Phase: ACN-H₂O (0.1% TFA), Gradient: 10%-65%) to give the product (39.5 mg, 55.5% yield) as a white solid.

MS (ESI) calcd for C₅₆H₆₅N₁₁O₁₁S [M+H]⁺ m/z=1100.46, found 1100.1.

### Example 68

### Synthesis of Linker-Payload (Mc-GGFG-Methylene-Compound 169)

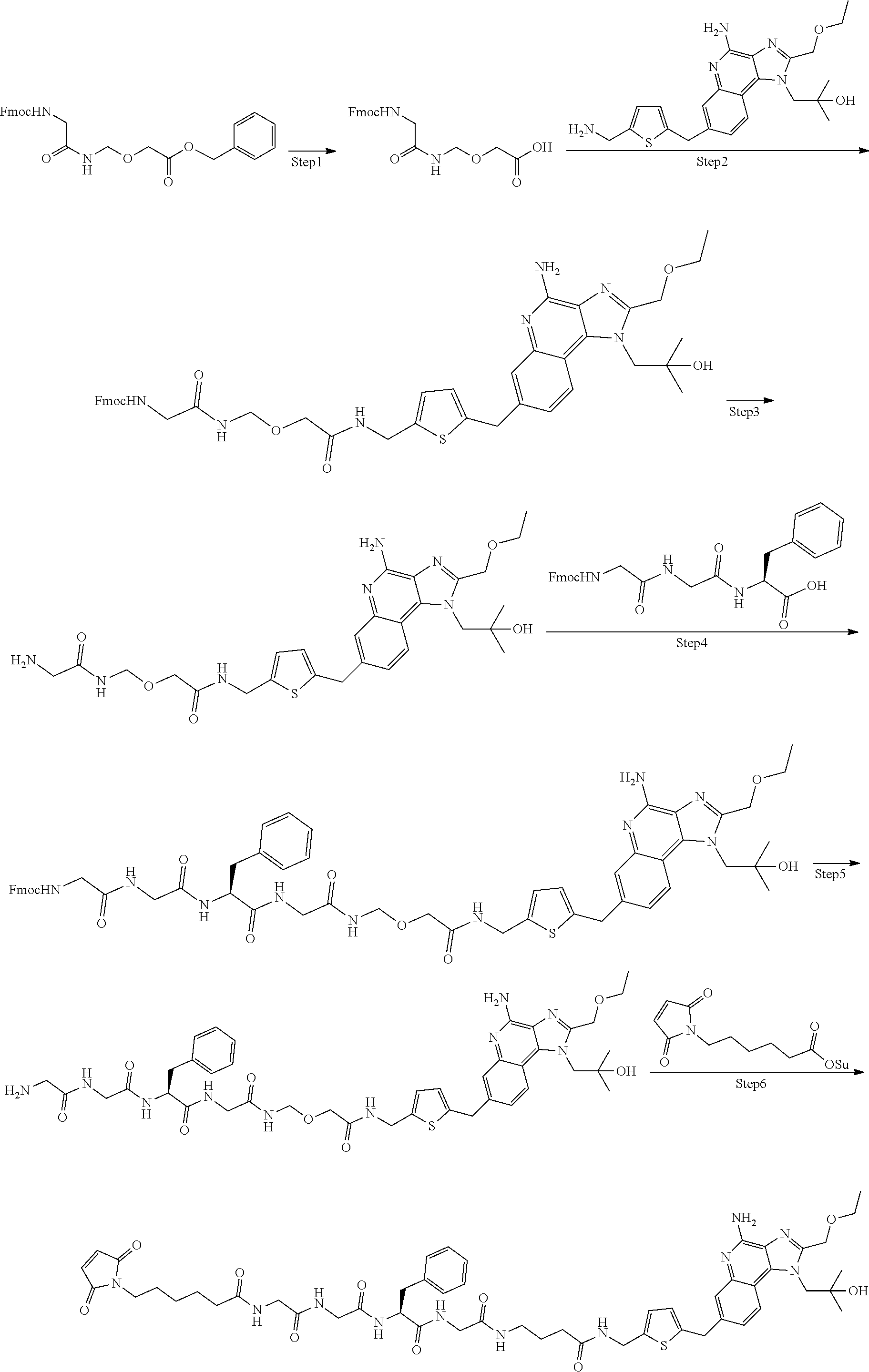

### Step 1. Preparation of 1-(9H fluoren-9 yl)-3, 6-dioxo-2, 9-dioxa-4, 7-diazaundecan-11-oic acid

A mixture of benzyl 1-(9H-fluoren-9-yl)-3,6-dioxo-2,9-dioxa-4,7-diazaundecan-11-oate (1 g, 2.1074 mmol) and 10% Pd/C (200 mg) in EtOH (20 mL) and EtOAc (10 mL) was stirred under H₂ (1 atm) at room temperature for 2 hours. HPLC showed the reaction was completed. The mixture was filtered through a Celite pad, and the filtrate was concentrated to give crude product (780 mg, 96.2% yield).

LCMS (ESI) calcd for C₂₀H₂₀N₂O₆ [M−H]⁻ m/z=383.13, found 383.2.

### Step 2. Preparation of (9H-fluoren-9-yl)methyl (2-(((2-(((5-((4-amino-2-(ethoxymethyl)-1-(2-hydroxy-2-methylpropyl)-1H-imidazo[4,5-c]quinolin-7-yl)methyl)thiophen-2-yl)methyl)amino)-2-oxoethoxy)methyl)amino)-2-oxoethyl)carbamate

To a solution of 1-(9H-fluoren-9-yl)-3,6-dioxo-2,9-dioxa-4,7-diazaundecan-11-oic acid (70 mg, 0.1802 mmol) and HOSu (40.4 mg, 0.3511 mmol) in DMF (3 mL) was added EDCI (112.2 mg, 0.5852 mmol) and the mixture was stirred at room temperature for 2 hours. HPLC showed the reaction was OK. The reaction mixture was added 1-(4-amino-7-((5-(aminomethyl)thiophen-2-yl)methyl)-2-(ethoxymethyl)-1H-imidazo[4,5-c]quinolin-1-yl)-2-methylpropan-2-ol (102.9 mg, 0.2341 mmol) and DIEA (90.7 mg, 0.7023 mmol) at room temperature. HPLC showed the reaction was OK. The reaction mixture was diluted with H₂O (50 mL) and extracted with EtOAc (50 mL×2). The combined organic layers were washed with H2O (50 mL×2). The organic layers were dried over anhydrous Na₂SO₄ and concentrated to afford the crude product (140 mg, 74.2% yield).

LCMS (ESI) calcd for C₄₃H₄₇N₇O₇S [M+H]⁺ m/z=806.33, found 806.3.

### Step 3. Preparation of 2-amino-N-((2-(((5-((4-amino-2-(ethoxymethyl)-1-(2-hydroxy-2-methylpropyl)-1H-imidazo[4,5-c]quinolin-7-yl)methyl)thiophen-2-yl)methyl)amino)-2-oxoethoxy)methyl)acetamide

To a solution of (9H-fluoren-9-yl)methyl (2-(((2-(((5-((4-amino-2-(ethoxymethyl)-1-(2-hydroxy-2-methylpropyl)-1H-imidazo[4,5-c]quinoline-7-yl)methyl)thiophen-2-yl)methyl)amino)-2-oxoethoxy)methyl)amino)-2-oxoethyl) carbamate (140 mg, 0.1737 mmol) in DMF (1 mL) was added 10% piperidine in DMF (3 mL) solution and the mixture was stirred at room temperature for 1 hour. HPLC showed the reaction was OK. To MTBE (100 mL) the reaction solution was added dropwise and precipitated the solid. The suspension was stirred at room temperature for 20 minutes. Then was centrifuged and the solid was washed with MTBE (15 mL). The solid was evaporated to dryness to afford the product (75 mg, 73.9% yield).

LCMS (ESI) calcd for C₂₈H₃₇N₇O₅S [M+H]⁺ m/z=584.26, found 584.4.

### Step 4. Preparation of (9H-fluoren-9-yl)methyl (S)-(1-(5-((4-amino-2-(ethoxymethyl)-1-(2-hydroxy-2-methylpropyl)-1H-imidazo[4,5-c]quinolin-7-yl)methyl)thiophen-2-yl)-12-benzyl-3,8,11,14,17-pentaoxo-5-oxa-2,7,10,13,16-pentaazaoctadecan-18-yl)carbamate

To a solution of (((9H-fluoren-9-yl)methoxy)carbonyl)glycylglycyl-L-phenylalanine (127 mg, 0.2535 mmol) and HOSu (43.7 mg, 0.3802 mmol) in DMF (3 mL) was added EDCI (145.7 mg, 0.7605 mmol) and the mixture was stirred at room temperature for 2 hours. HPLC showed the reaction was OK. The reaction mixture was added 2-amino-N-((2-(((5-((4-amino-2-(ethoxymethyl)-1-(2-hydroxy-2-methylpropyl)-1H-imidazo[4,5-c]quinolin-7-yl)methyl)thiophen-2-yl)methyl)amino)-2-oxoethoxy)methyl)acetamide (185 mg, 0.3169 mmol) and DIEA (122.8 mg, 0.9508 mmol) at room temperature. HPLC showed the reaction was OK. The reaction mixture was diluted with H2O (50 mL) and extracted with EtOAc (50 mL×2). The combined organic layers were washed with H₂O (50 mL×2). The organic layers were dried over anhydrous Na₂SO₄ and concentrated to afford the crude product (290 mg, 91.0% yield).

LCMS (ESI) calcd for C₅₆H₆₂N₁₀O₁₀S [M+H]⁺ m/z=1067.44, found 1067.2.

### Step 5. Preparation of (S)—N-(2-(((2-(((5-((4-amino-2-(ethoxymethyl)-1-(2-hydroxy-2-methylpropyl)-1H-imidazo[4,5-c]quinolin-7-yl)methyl)thiophen-2-yl)methyl)amino)-2-oxoethoxy)methyl)amino)-2-oxoethyl)-2-(2-(2-aminoacetamido)acetamido)-3-phenylpropanamide

To a solution of (9H-fluoren-9-yl)methyl (S)-(1-(5-((4-amino-2-(ethoxymethyl)-1-(2-hydroxy-2-methylpropyl)-1H-imidazo[4,5-c]quinolin-7-yl)methyl)thiophen-2-yl)-12-benzyl-3,8,11,14,17-pentaoxo-5-oxa-2,7,10,13,16-pentaazaoctadecan-18-yl)carbamate (290 mg, 0.2717 mmol) in DMF (1 mL) was added 10% piperidine in DMF (3 mL) solution and the mixture was stirred at room temperature for 1 hour. HPLC showed the reaction was OK. To MTBE (100 mL) the reaction solution was added dropwise and precipitated the solid. The suspension was stirred at room temperature for 20 minutes. Then the suspension was centrifuged and the solid was washed with MTBE (15 mL). The solid was evaporated to dryness to afford the product (190 mg, 94.6% yield).

LCMS (ESI) calcd for C₄₁H₅₂N₁₀O₈S [M+H]⁺ m/z=845.37, found 845.4.

### Step 6. Preparation of (S)—N-(1-(5-((4-amino-2-(ethoxymethyl)-1-(2-hydroxy-2-methylpropyl)-1H-imidazo[4,5-c]quinolin-7-yl)methyl)thiophen-2-yl)-12-benzyl-3,8,11,14,17-pentaoxo-5-oxa-2,7,10,13,16-pentaazaoctadecan-18-yl)-6-(2,5-dioxo-2,5-dihydro-1H-pyrrol-1-yl)hexanamide

To a solution of (S)—N-(2-(((2-(((5-((4-amino-2-(ethoxymethyl)-1-(2-hydroxy-2-methylpropyl)-1H-imidazo[4,5-c]quinolin-7-yl)methyl)thiophen-2-yl)methyl)amino)-2-oxoethoxy)methyl)amino)-2-oxoethyl)-2-(2-(2-aminoacetamido)acetamido)-3-phenylpropanamide (100 mg, 0.1183 mmol) and 2,5-dioxopyrrolidin-1-yl 6-(2,5-dioxo-2,5-dihydro-1H-pyrrol-1-yl)hexanoate (54.7 mg, 0.1775 mmol) in DMF (2 mL) was added DIEA (45.8 mg, 0.3549 mmol) and the mixture was stirred at room temperature for 1 hour. HPLC showed the reaction was OK. The mixture was purified by Prep-HPLC (Column: Gemini-C18, 250×50 mm, 10 um; Mobile Phase: ACN-H₂O (0.1% TFA), Gradient: 10%-65%) to give the linker-payload (Mc-GGFG-Methylene-Compound 169) (95 mg, 77.3%% yield) as a white solid.

MS (ESI) calcd for C₅₁H₆₃N₁₁O₁₁S [M+H]⁺ m/z=1038.44, found 1038.1.

### Example 69

### Synthesis of Linker-Payload (Mal-PEG4-GGFG-Methylene-Compound 169)

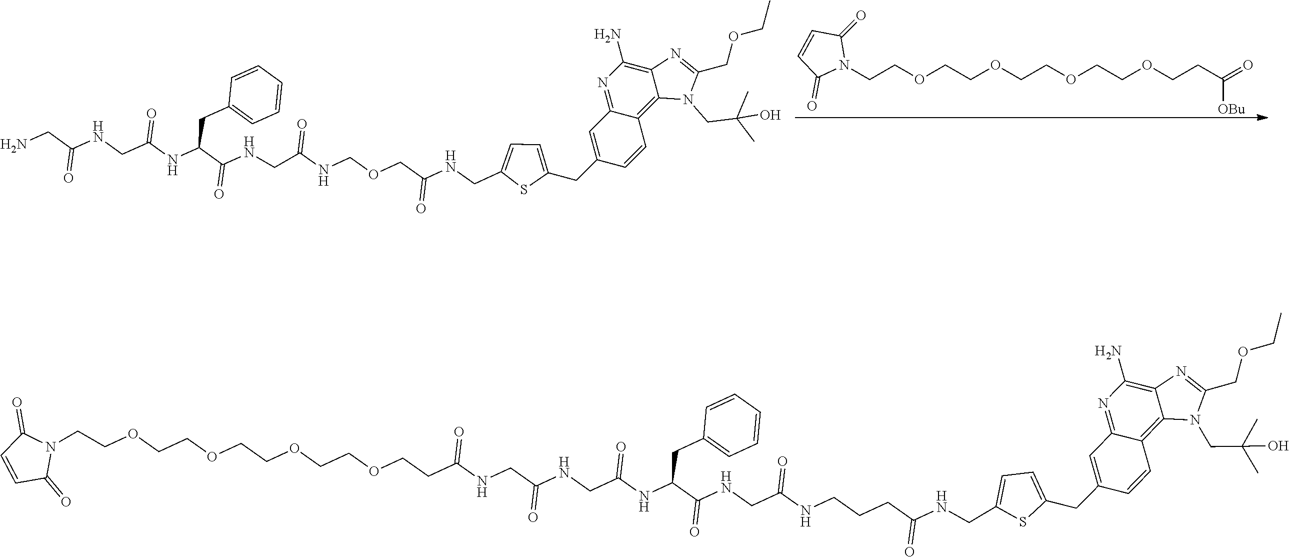

### Preparation of (S)—N-(1-(5-((4-amino-2-(ethoxymethyl)-1-(2-hydroxy-2-methylpropyl)-1H-imidazo[4,5-c]quinolin-7-yl)methyl)thiophen-2-yl)-12-benzyl-3,8,11,14,17-pentaoxo-5-oxa-2,7,10,13,16-pentaazaoctadecan-18-yl)-1-(2,5-dioxo-2,5-dihydro-1H-pyrrol-1-yl)-3,6,9,12-tetraoxapentadecan-15-amide

To a solution of (S)—N-(2-(((2-(((5-((4-amino-2-(ethoxymethyl)-1-(2-hydroxy-2-methylpropyl)-1H-imidazo[4,5-c]quinolin-7-yl)methyl)thiophen-2-yl)methyl)amino)-2-oxoethoxy)methyl)amino)-2-oxoethyl)-2-(2-(2-aminoacetamido)acetamido)-3-phenylpropanamide (90 mg, 0.1065 mmol) and 2,5-dioxopyrrolidin-1-yl 1-(2,5-dioxo-2,5-dihydro-1H-pyrrol-1-yl)-3,6,9,12-tetraoxapentadecan-15-oate (50 mg, 0.1130 mmol) in DMF (2 mL) was added DIEA (41.3 mg, 0.3195 mmol) and the mixture was stirred at room temperature for 1 hour. HPLC showed the reaction was OK. The mixture was purified by Prep-HPLC (Column: Gemini-C18, 250×50 mm, 10 um; Mobile Phase: ACN-H₂O (0.1% TFA), Gradient: 10%-65%) to give the product (80 mg, 64.1% yield) as a white solid.

MS (ESI) calcd for C₅₆H₇₃N₁₁O₁₅S [M+H]⁺ m/z=1172.50, found 1172.2.

Other linker-payload compounds were synthesized in similar ways as in Examples 63-69 except for using different reacting materials.

### Example 70

### hTLR7 and hTLR8 Cell Assays

The payload compounds of Formula (II′) were assayed to evaluate their agonistic activity on hTLR7 (human Toll-like receptor 7) and hTLR8 (human Toll-like receptor 8).

Reporter cells lines (HEK-Blue hTLR7 and HEK-Blue hTLR8) were obtained from InvivoGen. Cells were cryopreserved in liquid nitrogen per supplier's instruction. Upon test, the cells were thawed, passaged and cultured in the growth media of DMEM supplemented with 10% fetal bovine serum, 1×NEAA, 1 mM Pyruvate, 2 mM glutamine, 50 μg/mL penicillin, 50 U/mL streptomycin in the presence of the following antibiotics, as shown as Table 1.

TABLE 1
Reporter cell lines treated with antibiotics

| Cell line | Zeocin | Normocin | Blasticidin |
|---|---|---|---|
| HEK-Blue hTLR7 | 100 μg/mL | 100 μg/mL | 10 μg/mL |
| HEK-Blue hTLR8 | 100 μg/mL | 100 μg/mL | 30 μg/mL |

Test compounds (at desired concentrations diluted) were added to the 96-well assay plate, 0.5 μL per well. Reporter cells were harvested from the tissue culture flasks by incubation in PBS at 37° C. for two minutes after the media in the flask is removed and cells rinsed with PBS. Cells were counted and diluted in the HEK-Blue Detection media at 50000 cells/mL and incubated for 24 hours at 37° C., in a 5% CO₂ humidified incubator. Then 20 μL of cell supernatant were added to the assay plate containing the 180 L of QUANTI-Blue, incubated for 1 hour at 37° C. Optical density at 650 nm was analyzed using an Envision (Perkin Elmer) plate reader.

Table 2 shows the EC₅₀ potency for exemplary compounds.

TABLE 2

| | |
|---|---|
| Compound | EC₅₀ |

| No. | hTLR7* | hTLR8** |
|---|---|---|
| R848 | A | A′ |
| (Resquimod) | | |
| 1 | C | C′ |
| 2 | B | B′ |
| 3 | B | B′ |
| 4 | A | A′ |
| 5 | B | B′ |
| 6 | C | C′ |
| 7 | C | B′ |
| 8 | B | A′ |
| 9 | C | C′ |
| 10 | C | C′ |
| 11 | B | A′ |
| 12 | B | A′ |
| 13 | B | A′ |
| 14 | B | B′ |
| 15 | B | A′ |
| 16 | A | A′ |
| 17 | A | A′ |
| 18 | A | B′ |
| 19 | A | A′ |
| 20 | A | A′ |
| 21 | A | C′ |
| 22 | B | C′ |
| 23 | B | A′ |
| 24 | C | C′ |
| 25 | C | C′ |
| 26 | B | C′ |
| 27 | B | C′ |
| 28 | C | A′ |
| 29 | C | C′ |
| 30 | B | A′ |
| 31 | A | A′ |
| 32 | B | A′ |
| 33 | B | B′ |
| 34 | C | C′ |
| 35 | B | C′ |
| 36 | C | C′ |
| 37 | C | C′ |
| 38 | C | C′ |
| 39 | C | B′ |
| 40 | B | C′ |
| 41 | B | B′ |
| 42 | B | A′ |
| 43 | A | B′ |
| 44 | B | A′ |
| 45 | B | C′ |
| 46 | C | C′ |
| 47 | C | C′ |
| 48 | C | B′ |
| 49 | C | B′ |
| 50 | B | B′ |
| 51 | A | A′ |
| 52 | B | A′ |
| 53 | A | A′ |
| 54 | A | A′ |
| 55 | A | A′ |
| 56 | A | A′ |
| 57 | A | A′ |
| 58 | A | A′ |
| 59 | A | A′ |
| 60 | A | A′ |
| 61 | A | A′ |
| 62 | A | A′ |
| 63 | A | A′ |
| 64 | B | A′ |
| 65 | B | A′ |
| 66 | B | A′ |
| 67 | A | A′ |
| 68 | A | A′ |
| 69 | A | A′ |
| 70 | A | A′ |
| 71 | B | A′ |
| 72 | A | C′ |
| 73 | A | C′ |
| 147 | A | A′ |
| 148 | A | A′ |
| 149 | A | A′ |
| 150 | B | A′ |
| 151 | B | A′ |
| 152 | A | A′ |
| 153 | A | A′ |

*For hTLR7, “A” indicates a EC₅₀ value of less than 1 μM, “B” indicates a EC₅₀ value from 1 μM to 10 μM, and “C” indicates a EC₅₀ value of greater than 10 μM.
**For hTLR8, “A′” indicates a EC₅₀ value of less than 5 μM, “B′” indicates a EC₅₀ value from 5 μM to 20 μM, and “C′” indicates a EC₅₀ value of greater than 20 μM.

### Example 71

### PBMC Screening Assay

The payload compounds of Formula (II′) were assayed to evaluate the induction of TNF-α production in human PBMC (human peripheral blood mononuclear cells).

Human peripheral blood mononuclear cells (PBMC) were obtained from Allcells. PBMC were thawed, suspended at a concentration of 2.5×10⁶ cells/mL in growth media and 200 L was aliquoted into each well of a 96-well plate for a total of 50000 cells per well. PBMC were incubated for 24 hours at 37° C. in a 5% CO₂ humidified incubator. The following day PBMC plates were centrifuged at 500×g for 5 minutes and the growth media was removed. 200 μL of compound prepared in growth media at a series of diluted concentrations ranging from 2.54 nM to 50 μM were added to PBMC in duplicate and incubated for 24 hours at 37° C. in a 5% CO2 incubator. Prior to supernatant harvest, cells were spun at 500×g for 5 minutes to remove cell debris. TNF-α activity was assessed in the supernatant by ELISA (Elabscience) per the manufacturer's instructions. Optical density at 450 nm (ELISA) was analyzed using a plate reader. The EC₅₀ potency for the exemplary compounds provided herein are shown in Table 3.

TABLE 3

| | EC₅₀ (μM)* |
|---|---|
| Compound No. | TNF-α (hPBMC) |
| R848 (Resquimod) | 0.64 |
| 2 | B |
| 3 | B |
| 4 | B |
| 9 | A |
| 15 | A |
| 16 | A |
| 17 | B |
| 18 | B |
| 19 | A |
| 20 | A |
| 23 | B |
| 30 | B |
| 32 | B |
| 44 | B |
| 51 | B |
| 54 | B |
| 56 | A |
| 59 | A |
| 66 | B |
| 67 | A |
| 68 | A |
| 69 | A |
| 70 | A |
| 71 | B |
| 52 | B |
| 53 | A |
| 63 | A |
| 64 | A |
| 65 | A |
| 55 | A |
| 62 | A |
| 57 | A |
| 58 | A |
| 61 | A |
| 60 | A |
| 147 | A |
| 148 | A |
| 149 | A |
| 150 | A |
| 151 | A |
| 152 | A |
| 153 | A |
| 169 | B |
| 171 | A |

*“A” indicates a EC₅₀ value less than 0.64 μM, and “B” indicates a EC₅₀ value greater than 0.64 μM.

### Example 72

### Generation of Conjugate Compounds

### Cysteine Based Bioconjugation

The antibody is exchanged into an appropriate buffer, for example, phosphate, borate, PBS, or Tris-Acetate, at a concentration of about 2 mg/mL to about 10 mg/mL with an appropriate number of equivalents of a reducing agent, for example, dithiothreitol or tris(2-carboxyethyl)phosphine. The resultant solution is stirred for an appropriate amount of time and temperature to obtain the desired reduction. The linker-payload construct is added as a solution with stirring. Dependent on the physical properties of the linker-payload construct, a co-solvent is introduced prior to the addition of the linker-payload construct to facilitate solubility. The reaction is stirred at room temperature for about 1 hour to about 12 hours depending on the observed reactivity. The progression of the reaction is monitored by liquid chromatography-mass spectrometry (LC-MS). Once the reaction is deemed complete, the remaining free linker-payload construct is removed by applicable methods and the conjugate is exchanged into the desired formulation buffer. Such cysteine-based conjugates are synthesized starting with 1 mg of antibody construct (mAb) and 4 or 7 equivalents of linker-payload. Monomer content and drug-to-antibody ratios can be determined herein.

### Example 73

### Determination of Molar Ratio of Conjugate Compounds

This example illustrates one method by which the Drug-Antibody-Ratios (DARs) is determined by Hydrophobic Interaction Chromatography or LCMS method.

### Hydrophobic Interaction Chromatography Method

10 uL of a 5 mg/mL solution of the conjugate was injected into an HPLC system set-up with a TSKgel Butyl-NPR HIC column (2.5 uM particle size, 4.6 mm×3.5 mm). Then, over the course of 16 mins, a method run in which the mobile phase gradient ran from 100% mobile phase A to 100% mobile phase B over the course of 12 mins, followed by a 4 mins re-equilibration at 100% mobile phase A. The flow rate was 0.6 mL/min and detector was 280 nM. Mobile phase A was 1.5M ammonium sulfate, 50 mM K₂HPO₄ (pH7). Mobile phase B was 25% isopropanol in 25 mM K₂HPO₄ (pH7). Post-run the chromatograph was integrated and the molar ration was determined by summing the weighted peak area.

### Liquid Chromatography Mass Spectrometry (LCMS) Method

20 μg of Conjugates were added with 3 uL of 1M tris pH8.0, 2 uL of 0.1M DTT, and diluted with water to 30 uL. The mixture was incubated at 37° C. for 30 min to afford reduced conjugates. 2 μg of reduced conjugate is injected into an LC/MS Agilent Technologies 6224/6230 TOF coupled with Agilent 1260 HPLC system with (Agilent PLRP-S 1000A, 8 μm, 50×2.1 mm). Phase A was 0.05% TFA in H₂O. Phase B was 0.05% TFA in acetonitrile. Over the course of 10 mins, a method run in which the mobile phase gradient ran from 100% mobile phase A to 100% mobile phase B over the course of 7 mins, followed by a 3 mins re-equilibration at 75% mobile phase A. The flow rate was 0.5 mL/min and detector was 214 nm and 280 nM. Raw data is obtained and is deconvoluted with software. The average mass of Conjugates was calculated by the software, which used top peak height at 25% for the calculation. The average mass of intact conjugates is calculated by the software. This data is then imported into another program to calculate the molar ratio of the payload:conjugate.

### Example 74

### Synthesis of Conjugate 1

This example demonstrates the synthesis of Immunoconjugate 1 with trastuzumab as the antibody construct (Tras).

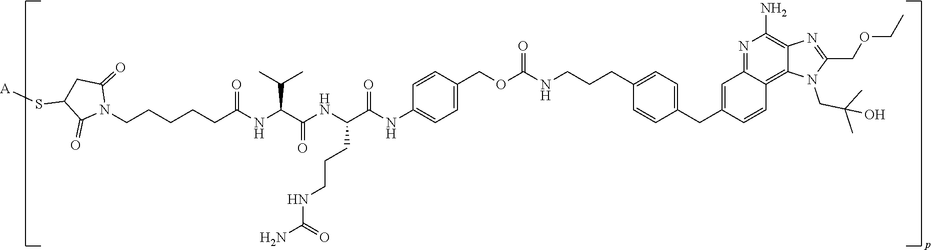

To the solutions of Trastuzumab in bacteriostatic water for injection, 5 mM TCEP was added to the target molecular ratio. Final concentration of Trastuzumab was 10 mg/mL. The reaction mixture was incubated at 37′C for 2 hours. Then DMA and 10 mg/mL Mc-VC-PAB-Compound 3 diluted in DMA was added. Final content of DMA in the reaction system was 10% and final molar ratio of Mc-VC-PAB-Compound 3 was 4. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-antibody ratio (DAR) was determined by liquid chromatography mass spectrometry analysis. Conjugate 1 had a DAR of 2.28.

### Example 75

### Synthesis of Conjugate 2

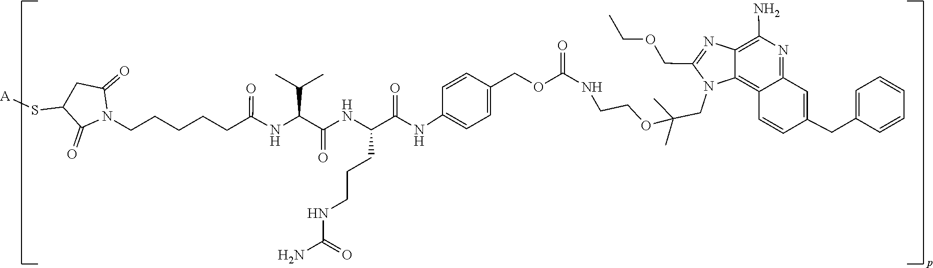

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 23 diluted in DMA was added. Final content of DMA in the reaction system was 10% and final molar ratio of Mc-VC-PAB-Compound 23 was 4. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 2 had a DAR of 2.10.

### Example 76

### Synthesis of Conjugate 3

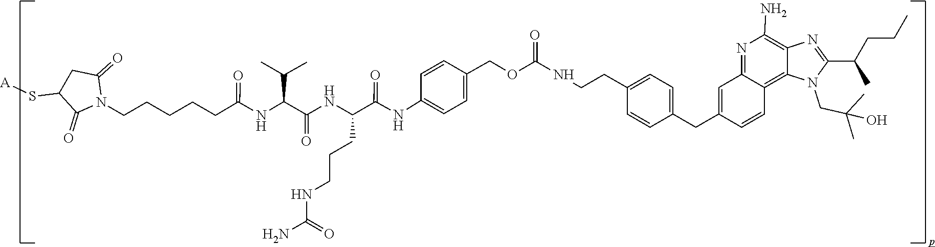

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 9 diluted in DMA was added. Final content of DMA in the reaction system was 10% and final molar ratio of Mc-VC-PAB-Compound 9 was 4. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 3 had a DAR of 2.07.

### Example 77

### Synthesis of Conjugate 4

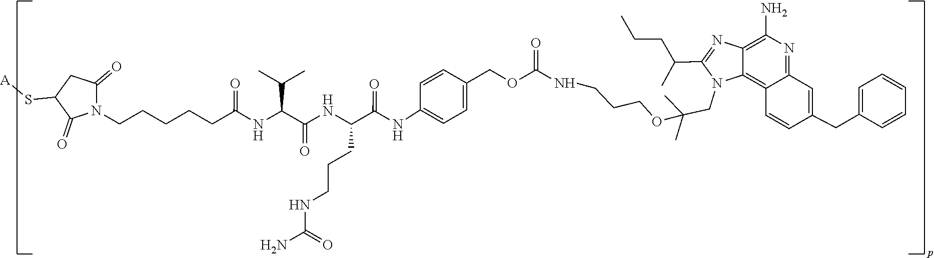

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 32 diluted in DMA was added. Final content of DMA in the reaction system was 10% and final molar ratio of Mc-VC-PAB-Compound 32 was 4. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 4 had a DAR of 2.01.

### Example 78

### Synthesis of Conjugate 5

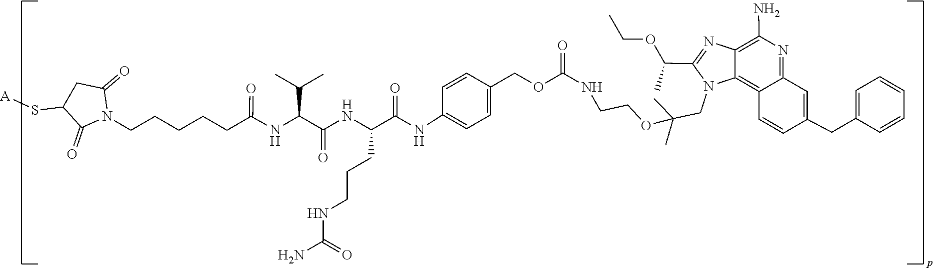

To the solutions of Trastuzumab in bacteriostatic water for injection, 5 mM TCEP was added to the target molecular ratio. Final concentration of Trastuzumab was 10 mg/mL. The reaction mixture was (pH buffer controlled by PBS) incubated at 37° C. for 2 hours. Then DMA and 10 mg/mL Mc-VC-PAB-Compound 30 diluted in DMA was added. Final content of DMA in the reaction system was 10% and final molar ratio of Mc-VC-PAB-Compound 30 was 4. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 5 had a DAR of 1.93.

### Example 79

### Synthesis of Conjugate 6

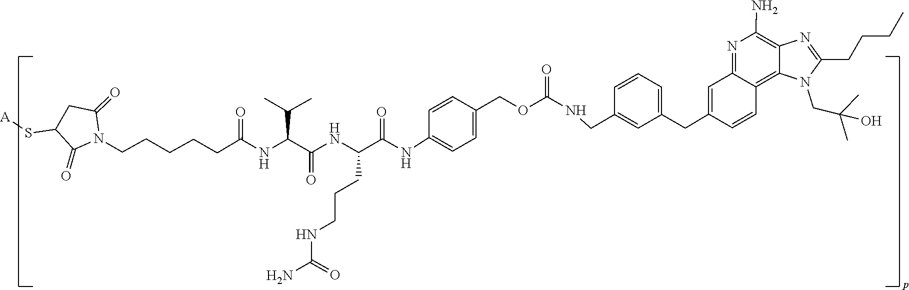

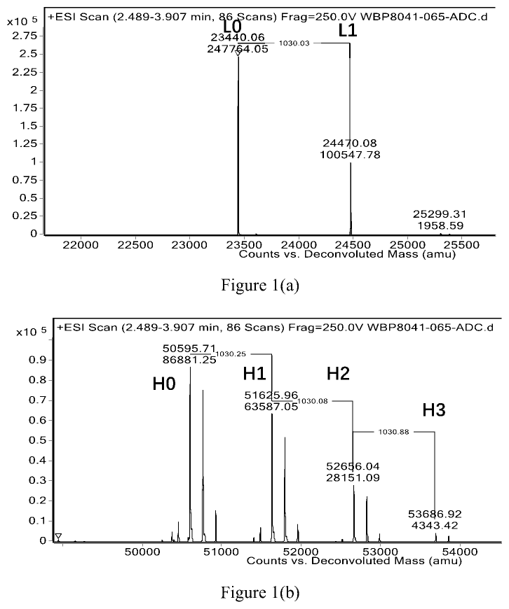

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 16 diluted in DMA was added. Final content of DMA in the reaction system was 10% and final molar ratio of Mc-VC-PAB-Compound 16 was 4. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. FIGS. 1(a) and 1(b) show the liquid chromatography mass spectrometry results for Conjugate 6, indicating that the DAR of Conjugate 6 is 2.02.

### Example 80

### Synthesis of Conjugate 7

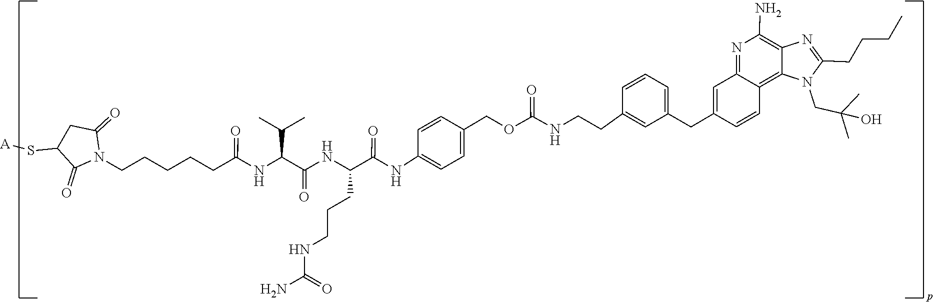

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 17 diluted in DMA was added. Final content of DMA in the reaction system was 10% and final molar ratio of Mc-VC-PAB-Compound 17 was 4. The reaction mixture was incubated at 4TC for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 7 had a DAR of 1.89.

### Example 81

### Synthesis of Conjugate 8

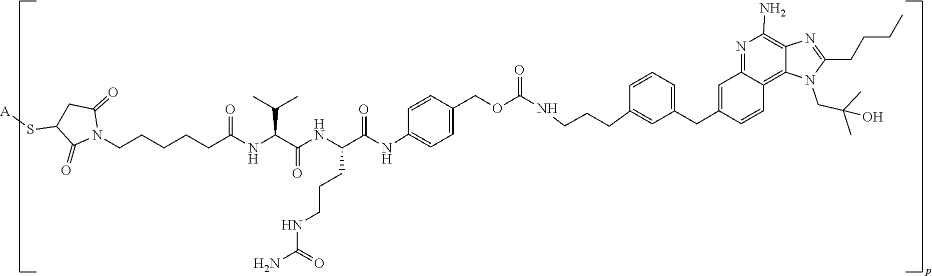

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 18 diluted in DMA was added. Final content of DMA in the reaction system was 10% and final molar ratio of Mc-VC-PAB-Compound 18 was 4. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 8 had a DAR of 2.04.

### Example 82

### Synthesis of Conjugate 9

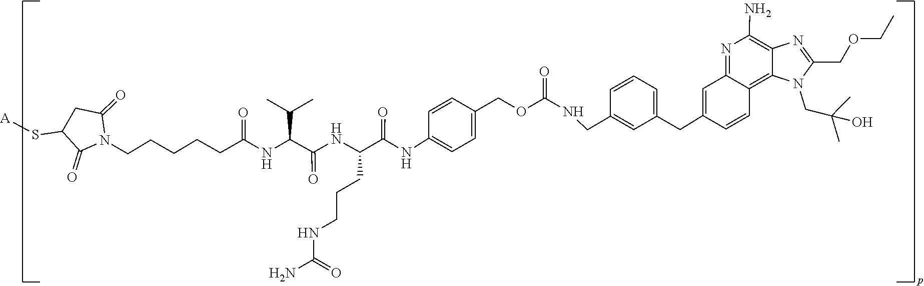

To the solutions of Trastuzumab in bacteriostatic water for injection, 5 mM TCEP was added to the target molecular ratio. Final concentration of Trastuzumab was 10 mg/mL. The reaction mixture was incubated at 37TC for 2 hours. Then DMA and 10 mg/mL Mc-VC-PAB-Compound 4 diluted in DMA was added. Final content of DMA in the reaction system was 10% and final molar ratio of Mc-VC-PAB-Compound 4 was 4. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 9 had a DAR of 2.05.

### Example 83

### Synthesis of Conjugate 10

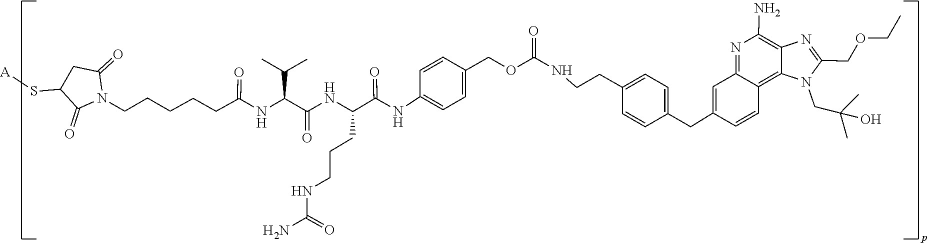

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 2 diluted in DMA was added. Final content of DMA in the reaction system was 10% and final molar ratio of Mc-VC-PAB-Compound 2 was 4. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 10 had a DAR of 2.30.

### Example 84

### Synthesis of Conjugate 11

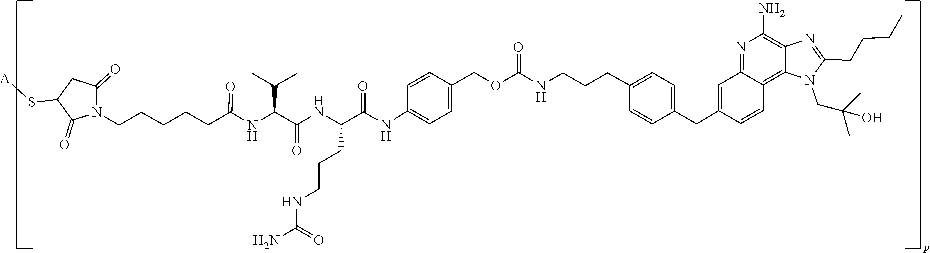

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 15 diluted in DMA was added. Final content of DMA in the reaction system was 10% and final molar ratio of Mc-VC-PAB-Compound 15 was 4. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 11 had a DAR of 2.13.

### Example 85

### Synthesis of Conjugate 12

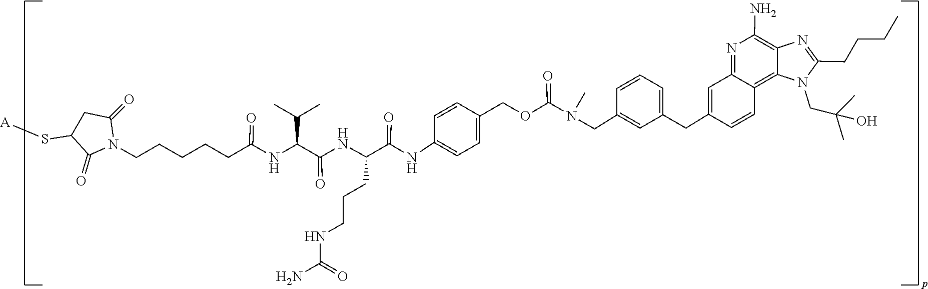

To the solutions of Trastuzumab in bacteriostatic water for injection, 5 mM TCEP was added to the target molecular ratio. Final concentration of Trastuzumab was 10 mg/mL. The reaction mixture was incubated at 37TC for 2 hours. Then DMA and 10 mg/mL Mc-VC-PAB-Compound 20 diluted in DMA was added. Final content of DMA in the reaction system was 10% and final molar ratio of Mc-VC-PAB-Compound 20 was 4. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-antibody ratio (DAR) was determined by liquid chromatography mass spectrometry analysis. Conjugate 12 had a DAR of 1.91.

### Example 86

### Synthesis of Conjugate 13

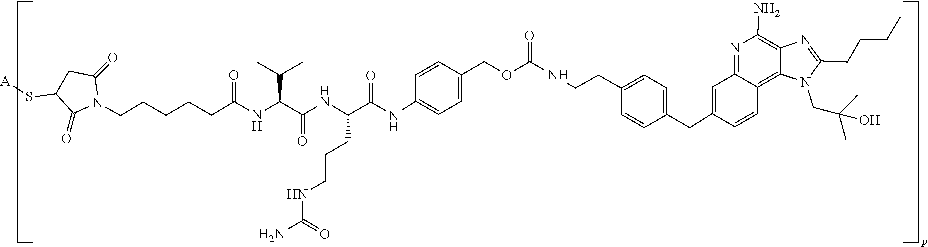

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 19 diluted in DMA was added. Final content of DMA in the reaction system was 10% and final molar ratio of Mc-VC-PAB-Compound 19 was 4. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-antibody ratio (DAR) was determined by liquid chromatography mass spectrometry analysis. Conjugate 13 had a DAR of 1.94.

### Example 87

### Synthesis of Conjugate 14

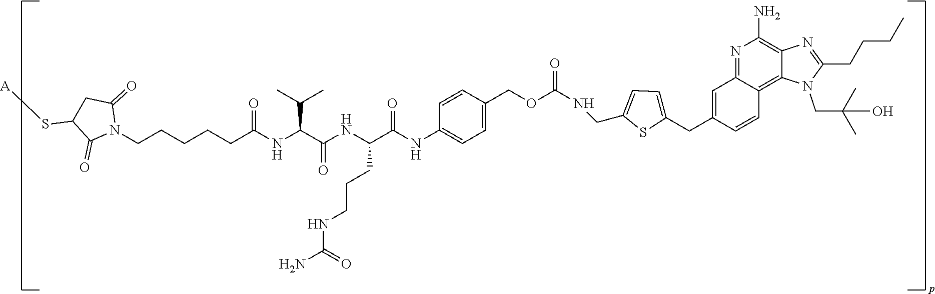

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 69 diluted in DMA was added. Final content of DMA in the reaction system was 10% and final molar ratio of Mc-VC-PAB-Compound 69 was 4. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 14 had a DAR of 1.99.

### Example 88

### Synthesis of Conjugate 15

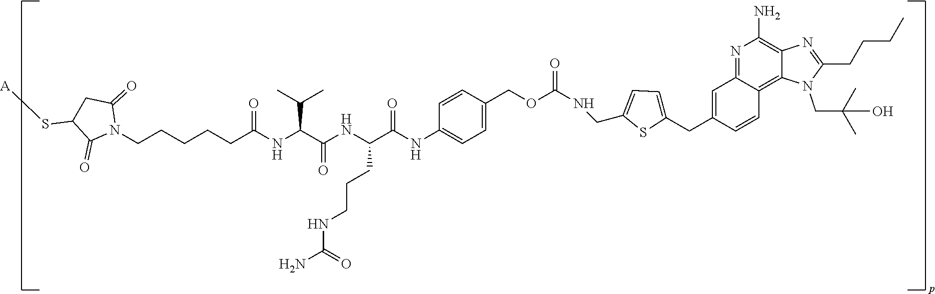

To the solutions of Trastuzumab in bacteriostatic water for injection, 5 mM TCEP was added to the target molecular ratio. Final concentration of Trastuzumab was 10 mg/mL. The reaction mixture was incubated at 37TC for 2 hours. Then DMA and 10 mg/mL Mc-VC-PAB-Compound 69 diluted in DMA was added. Final content of DMA in the reaction system was 10% and final molar ratio of Mc-VC-PAB-Compound 69 was 7. The reaction mixture was incubated at 4° C.T for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 15 had a DAR of 3.9.

### Example 89

### Synthesis of Conjugate 16

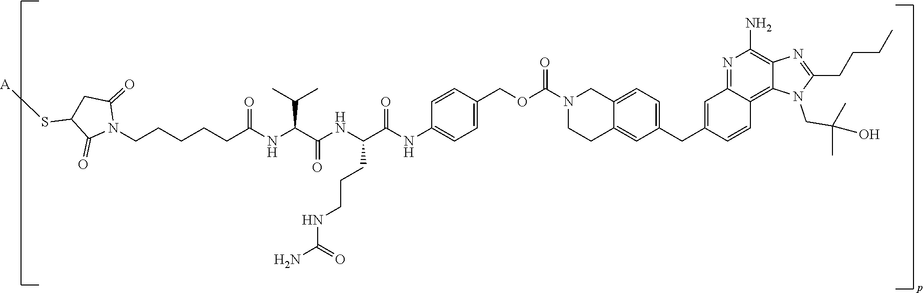

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 59 diluted in DMA was added. Final content of DMA in the reaction system was 10% and final molar ratio of Mc-VC-PAB-Compound 59 was 4. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 16 had a DAR of 1.82.

### Example 90

### Synthesis of Conjugate 17

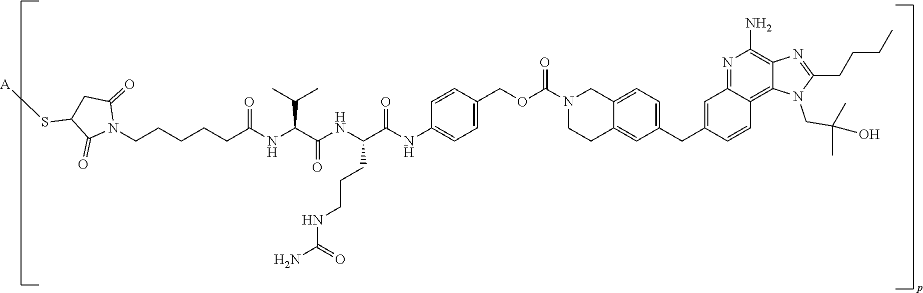

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 59 diluted in DMA was added. Final content of DMA in the reaction system was 10% and final molar ratio of Mc-VC-PAB-Compound 59 was 7. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 17 had a DAR of 3.71.

### Example 91

### Synthesis of Conjugate 18

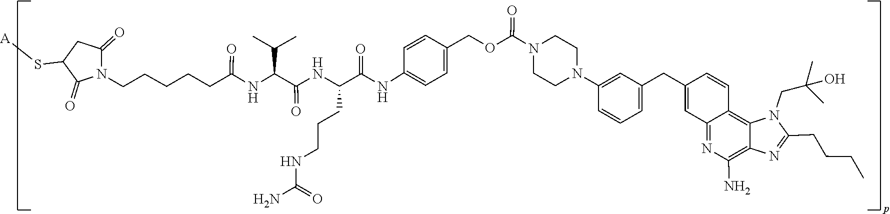

To the solutions of Trastuzumab in bacteriostatic water for injection, 5 mM TCEP was added to the target molecular ratio. Final concentration of Trastuzumab was 10 mg/mL. The reaction mixture was incubated at 37TC for 2 hours. Then DMA and 10 mg/mL Mc-VC-PAB-Compound 63 diluted in DMA was added. Final content of DMA in the reaction system was 10% and final molar ratio of Mc-VC-PAB-Compound 63 was 4. The reaction mixture was incubated at 4TC for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 18 had a DAR of 2.12.

### Example 92

### Synthesis of Conjugate 19

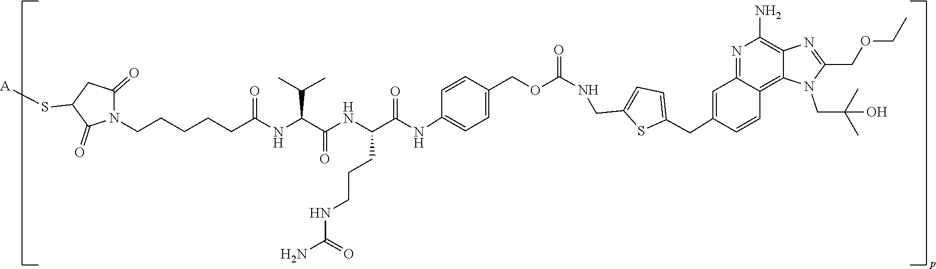

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 147 diluted in DMA was added. Final content of DMA in the reaction system was 10% and final molar ratio of Mc-VC-PAB-Compound 147 was 4. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 19 had a DAR of 1.75.

### Example 93

### Synthesis of Conjugate 20

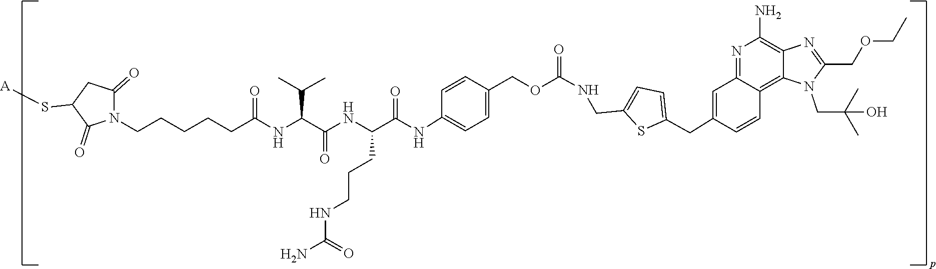

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 147 diluted in DMA was added. Final content of DMA in the reaction system was 10% and final molar ratio of Mc-VC-PAB-Compound 147 was 7. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 20 had a DAR of 4.06.

### Example 94

### Synthesis of Conjugate 21

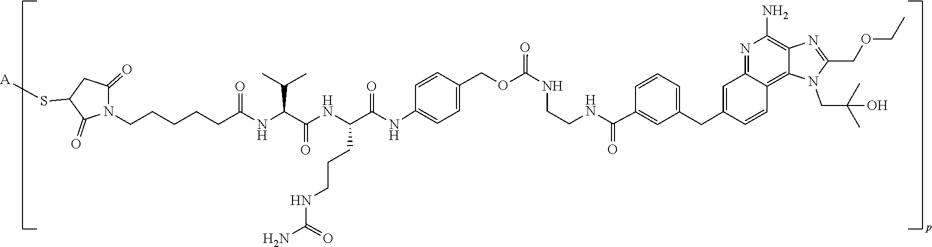

To the solutions of Trastuzumab in bacteriostatic water for injection, 5 mM TCEP was added to the target molecular ratio. Final concentration of Trastuzumab was 10 mg/mL. The reaction mixture was incubated at 37TC for 2 hours. Then DMA and 10 mg/mL Mc-VC-PAB-Compound 151 diluted in DMA was added. Final content of DMA in the reaction system was 10% and final molar ratio of Mc-VC-PAB-Compound 151 was 4. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 21 had a DAR of 2.02

### Example 95

### Synthesis of Conjugate 22

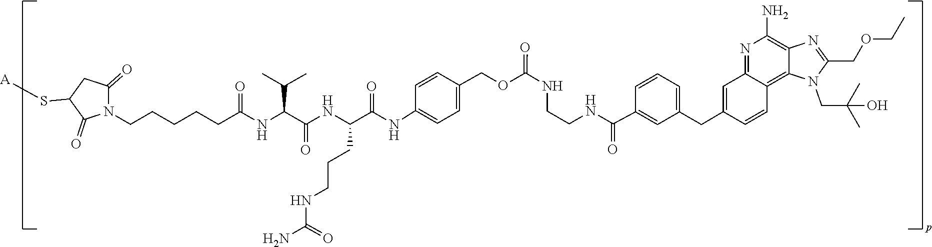

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 151 diluted in DMA was added. Final content of DMA in the reaction system was 10% and final molar ratio of Mc-VC-PAB-Compound 151 was 7. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 22 had a DAR of 3.85.

### Example 96

### Synthesis of Conjugate 23

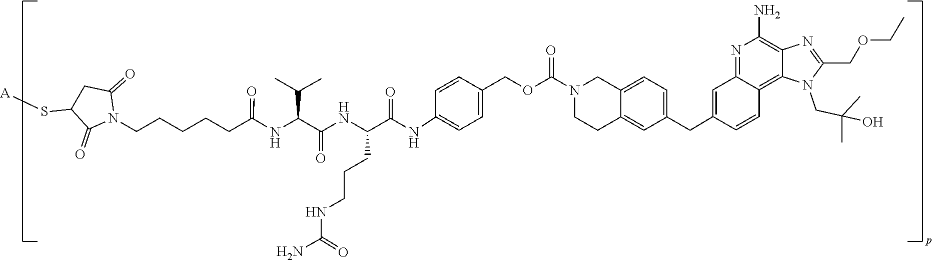

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 152 diluted in DMA was added. Final content of DMA in the reaction system was 10% and final molar ratio of Mc-VC-PAB-Compound 152 was 4. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 23 had a DAR of 1.76.

### Example 97

### Synthesis of Conjugate 24

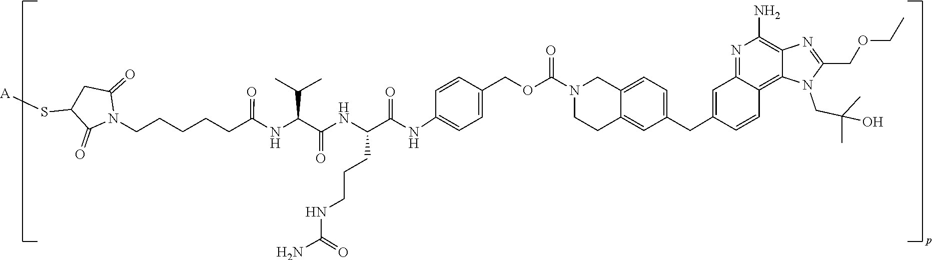

To the solutions of Trastuzumab in bacteriostatic water for injection, 5 mM TCEP was added to the target molecular ratio. Final concentration of Trastuzumab was 10 mg/mL. The reaction mixture was incubated at 37TC for 2 hours. Then DMA and 10 mg/mL Mc-VC-PAB-Compound 152 diluted in DMA was added. Final content of DMA in the reaction system was 10% and final molar ratio of Mc-VC-PAB-Compound 152 was 7. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 24 had a DAR of 3.92.

### Example 98

### Synthesis of Conjugate 25

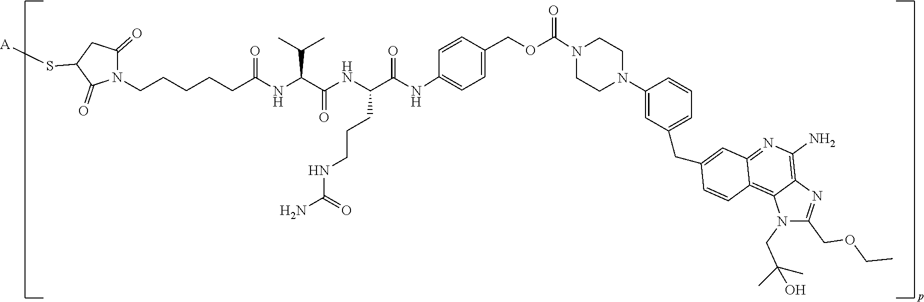

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 153 diluted in DMA was added. Final content of DMA in the reaction system was 10% and final molar ratio of Mc-VC-PAB-Compound 153 was 4. The reaction mixture was incubated at 4′C for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 25 had a DAR of 1.98.

### Example 99

### Synthesis of Conjugate 26

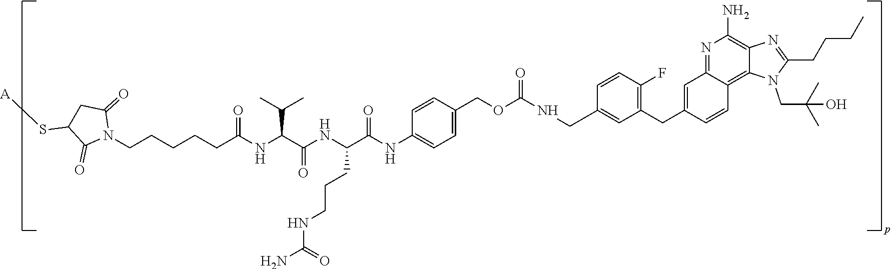

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 67 diluted in DMA was added. Final content of DMA in the reaction system was 10% and final molar ratio of Mc-VC-PAB-Compound 67 was 4. The reaction mixture was incubated at 4° C.C for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 26 had a DAR of 2.12.

### Example 100

### Synthesis of Conjugate 27

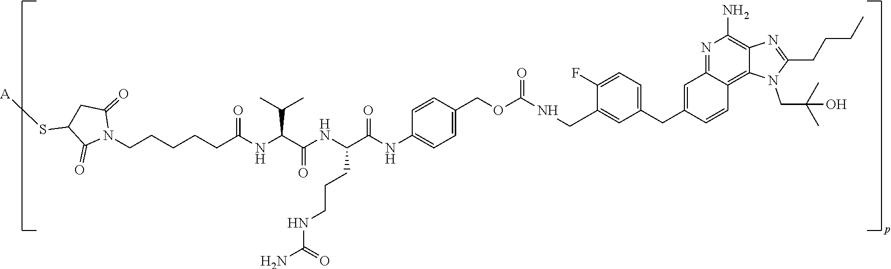

To the solutions of Trastuzumab in bacteriostatic water for injection, 5 mM TCEP was added to the target molecular ratio. Final concentration of Trastuzumab was 10 mg/mL. The reaction mixture was incubated at 37TC for 2 hours. Then DMA and 10 mg/mL Mc-VC-PAB-Compound 68 diluted in DMA was added. Final content of DMA in the reaction system was 10% and final molar ratio of Mc-VC-PAB-Compound 68 was 4. The reaction mixture was incubated at 4° C.C for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 27 had a DAR of 1.96.

### Example 101

### Synthesis of Conjugate 28

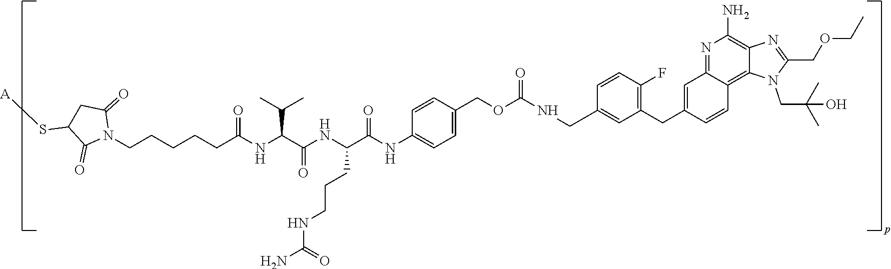

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 148 diluted in DMA was added. Final content of DMA in the reaction system was 10% and final molar ratio of Mc-VC-PAB-Compound 148 was 4. The reaction mixture was incubated at 4′C for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 28 had a DAR of 1.97.

### Example 102

### Synthesis of Conjugate 29

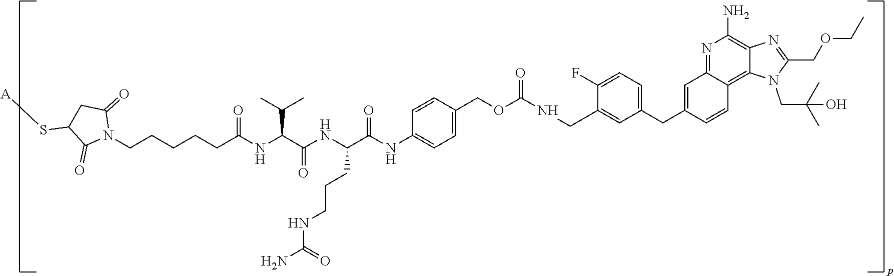

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 149 diluted in DMA was added. Final content of DMA in the reaction system was 10% and final molar ratio of Mc-VC-PAB-Compound 149 was 4. The reaction mixture was incubated at 4′C for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 29 had a DAR of 1.65.

### Example 103

### Synthesis of Conjugate 30

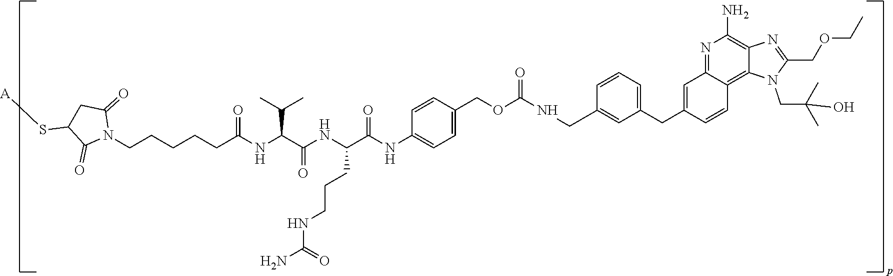

To the solutions of Trastuzumab in bacteriostatic water for injection, 5 mM TCEP was added to the target molecular ratio. Final concentration of Trastuzumab was 10 mg/mL. The reaction mixture was incubated at 37TC for 2 hours. Then DMA and 10 mg/mL Mc-VC-PAB-Compound 4 diluted in DMA was added. Final content of DMA in the reaction system was 10% and final molar ratio of Mc-VC-PAB-Compound 4 was 4. The reaction mixture was incubated at 4TC for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 30 had a DAR of 2.12.

### Example 104

### Synthesis of Conjugate 31

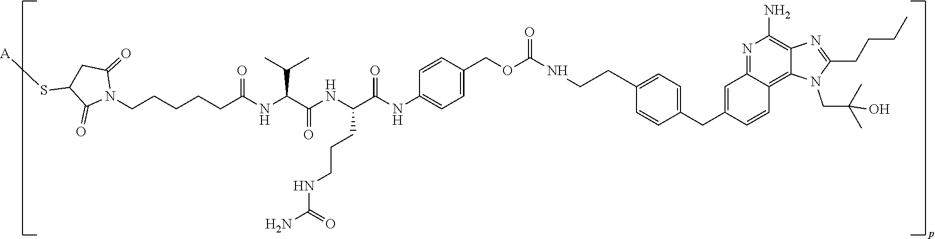

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 19 diluted in DMA was added. Final content of DMA in the reaction system was 10% and final molar ratio of Mc-VC-PAB-Compound 19 was 4. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 31 had a DAR of 1.88.

### Example 105

### Synthesis of Conjugate 32

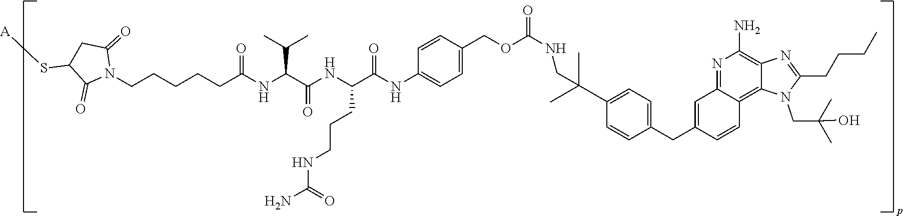

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 70 diluted in DMA was added. Final content of DMA in the reaction system was 10% and final molar ratio of Mc-VC-PAB-Compound 70 was 7. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 32 had a DAR of 4.15.

### Example 106

### Synthesis of Conjugate 33

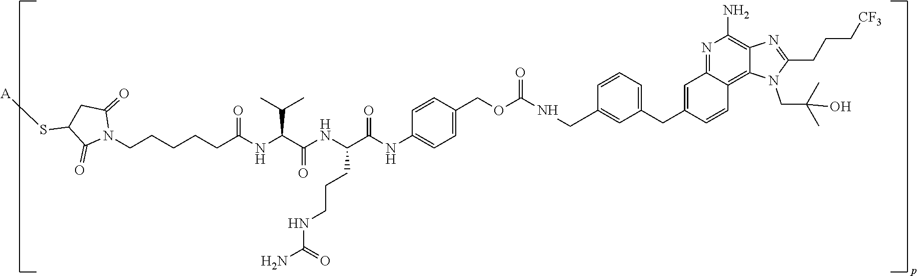

To the solutions of Trastuzumab in bacteriostatic water for injection, 5 mM TCEP was added to the target molecular ratio. Final concentration of Trastuzumab was 10 mg/mL. The reaction mixture was incubated at 37TC for 2 hours. Then DMA and 10 mg/mL Mc-VC-PAB-Compound 51 diluted in DMA was added. Final content of DMA in the reaction system was 10% and final molar ratio of Mc-VC-PAB-Compound 51 was 7. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 33 had a DAR of 3.93.

### Example 107

### Synthesis of Conjugate 34

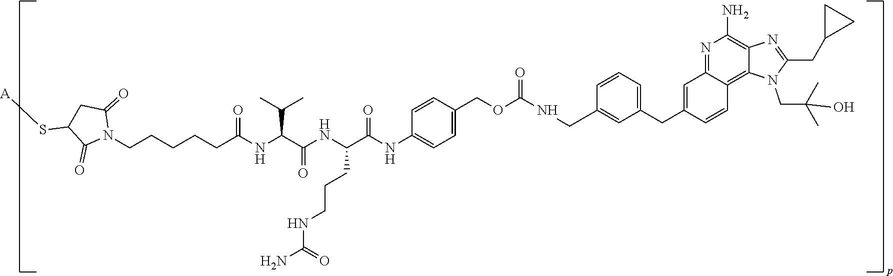

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 54 diluted in DMA was added. Final content of DMA in the reaction system was 10% and final molar ratio of Mc-VC-PAB-Compound 54 was 7. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 34 had a DAR of 3.89.

### Example 108

### Synthesis of Conjugate 35

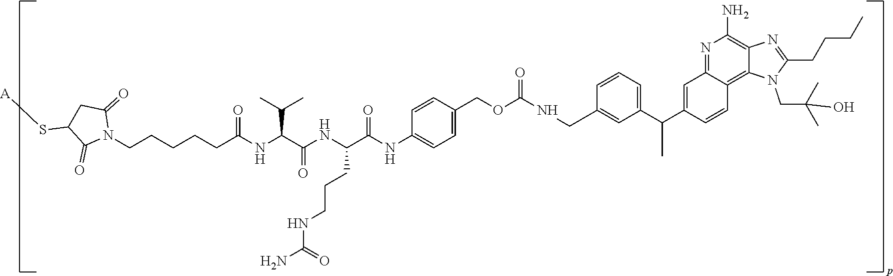

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 56 diluted in DMA was added. Final content of DMA in the reaction system was 10% and final molar ratio of Mc-VC-PAB-Compound 56 was 7. The reaction mixture was incubated at 4° C.C for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 35 had a DAR of 4.26.

### Example 109

### Synthesis of Conjugate 36

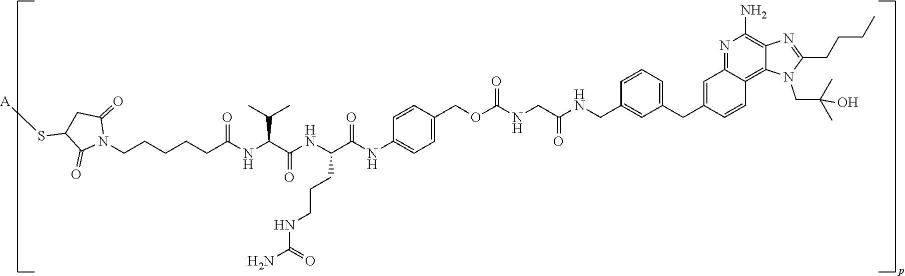

To the solutions of Trastuzumab in bacteriostatic water for injection, 5 mM TCEP was added to the target molecular ratio. Final concentration of Trastuzumab was 10 mg/mL. The reaction mixture was incubated at 37TC for 2 hours. Then DMA and 10 mg/mL Mc-VC-PAB-Compound 66 diluted in DMA was added. Final content of DMA in the reaction system was 10% and final molar ratio of Mc-VC-PAB-Compound 66 was 4. The reaction mixture was incubated at 4° C.C for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 36 had a DAR of 1.92.

### Example 110

### Synthesis of Conjugate 37

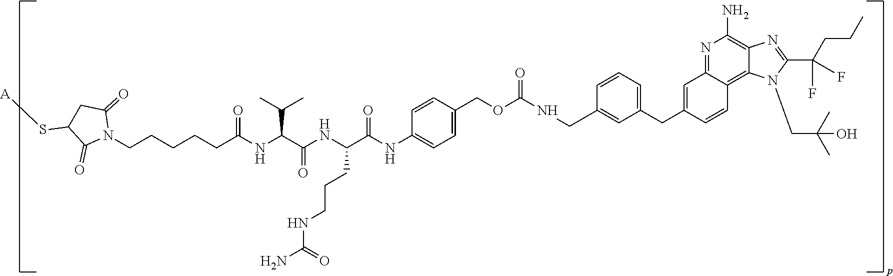

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 52 diluted in DMA was added. Final content of DMA in the reaction system was 10% and final molar ratio of Mc-VC-PAB-Compound 52 was 4. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 37 had a DAR of 1.88.

### Example 111

### Synthesis of Conjugate 38

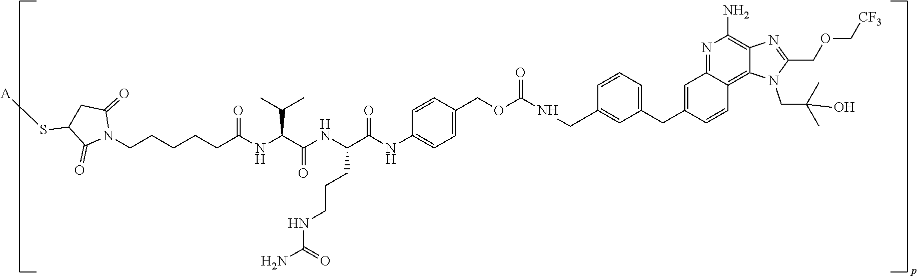

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 53 diluted in DMA was added. Final content of DMA in the reaction system was 10% and final molar ratio of Mc-VC-PAB-Compound 53 was 7. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 38 had a DAR of 3.83.

### Example 112

### Synthesis of Conjugate 39

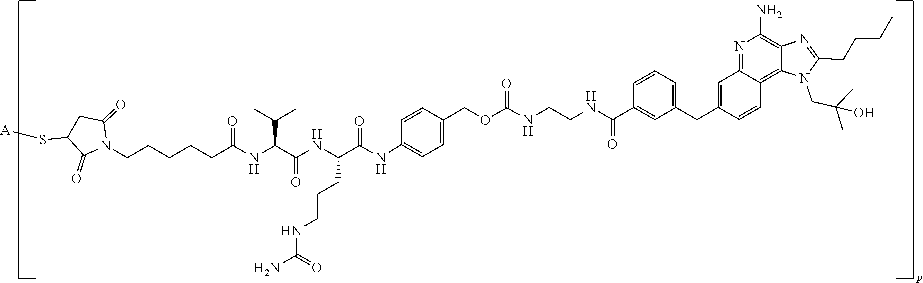

To the solutions of Trastuzumab in bacteriostatic water for injection, 5 mM TCEP was added to the target molecular ratio. Final concentration of Trastuzumab was 10 mg/mL. The reaction mixture was incubated at 37TC for 2 hours. Then DMA and 10 mg/mL Mc-VC-PAB-Compound 64 diluted in DMA was added. Final content of DMA in the reaction system was 10% and final molar ratio of Mc-VC-PAB-Compound 64 was 4. The reaction mixture was incubated at 4° C.T for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 39 had a DAR of 1.92.

### Example 113

### Synthesis of Conjugate 40

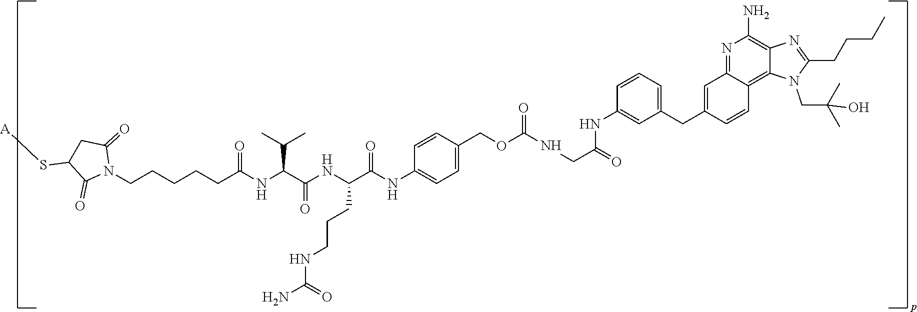

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 65 diluted in DMA was added. Final content of DMA in the reaction system was 10% and final molar ratio of Mc-VC-PAB-Compound 65 was 7. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 40 had a DAR of 3.86.

### Example 114

### Synthesis of Conjugate 41

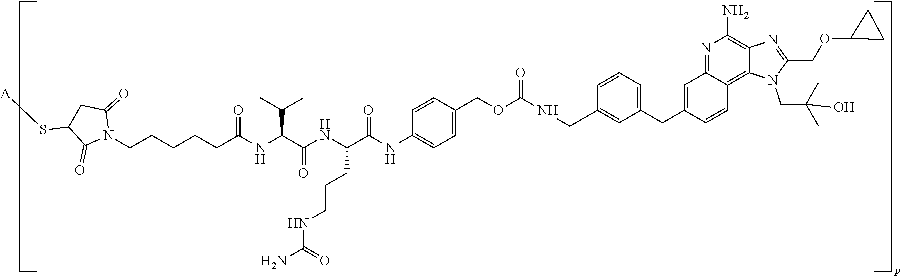

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 55 diluted in DMA was added. Final content of DMA in the reaction system was 10% and final molar ratio of Mc-VC-PAB-Compound 55 was 7. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 41 had a DAR of 3.96.

### Example 115

### Synthesis of Conjugate 42

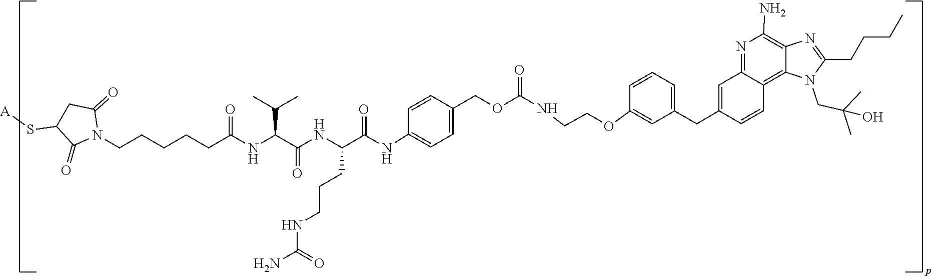

To the solutions of Trastuzumab in bacteriostatic water for injection, 5 mM TCEP was added to the target molecular ratio. Final concentration of Trastuzumab was 10 mg/mL. The reaction mixture was incubated at 37TC for 2 hours. Then DMA and 10 mg/mL Mc-VC-PAB-Compound 62 diluted in DMA was added. Final content of DMA in the reaction system was 10% and final molar ratio of Mc-VC-PAB-Compound 62 was 7. The reaction mixture was incubated at 4° C.T for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 43 had a DAR of 3.89.

### Example 116

### Synthesis of Conjugate 43

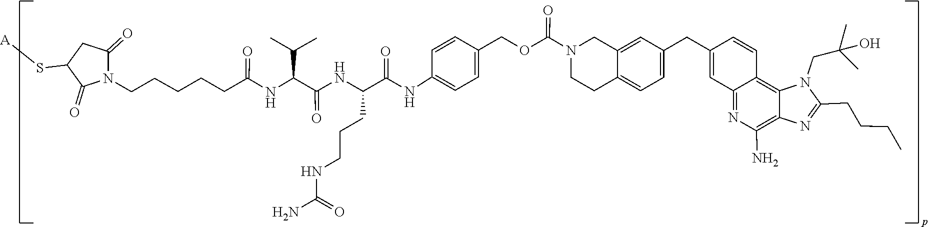

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-VC-PAB-Compound 60 diluted in DMA was added. Final content of DMA in the reaction system was 10% and final molar ratio of Mc-VC-PAB-Compound 60 was 7. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 43 had a DAR of 3.93.

### Example 117

### Synthesis of Conjugate 44

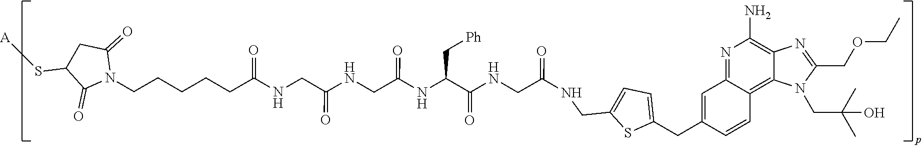

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-GGFG-Compound 147 diluted in DMA was added. Final content of DMA in the reaction system was 10% and final molar ratio of Mc-GGFG-Compound 147 was 7. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 44 had a DAR of 3.70.

### Example 118

### Synthesis of Conjugate 45

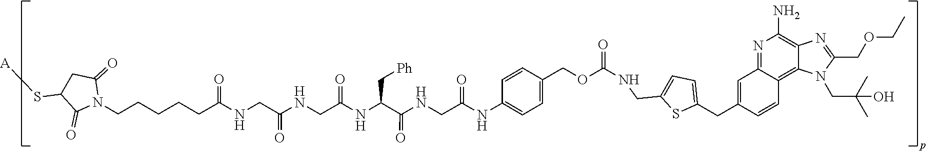

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-GGFG-PAB-Compound 147 diluted in DMA was added. Final content of DMA in the reaction system was 10% and final molar ratio of Mc-GGFG-PAB-Compound 147 was 7. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 44 had a DAR of 4.12.

### Example 119

### Synthesis of Conjugate 46

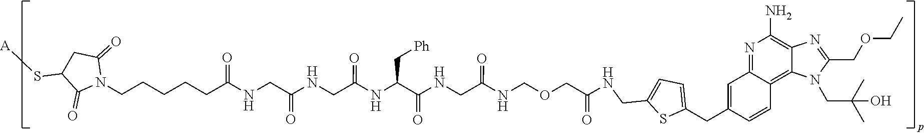

To the solutions of Trastuzumab in bacteriostatic water for injection, 5 mM TCEP was added to the target molecular ratio. Final concentration of Trastuzumab was 10 mg/mL. The reaction mixture was incubated at 37° C. for 2 hours. Then DMA and 10 mg/mL Mc-GGFG-Methylene-Compound 147 diluted in DMA was added. Final content of DMA in the reaction system was 10% and final molar ratio of Mc-GGFG-Methylene-Compound 147 was 7. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 46 had a DAR of 4.01.

### Example 120

### Synthesis of Conjugate 47

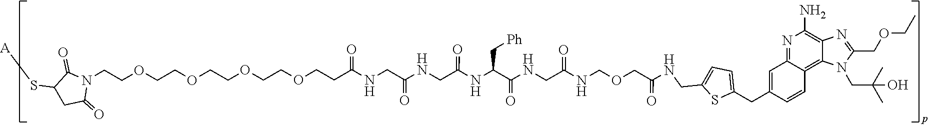

To the solutions of Trastuzumab in bacteriostatic water for injection, 5 mM TCEP was added to the target molecular ratio. Final concentration of Trastuzumab was 10 mg/mL. The reaction mixture was incubated at 37TC for 2 hours. Then DMA and 10 mg/mL Mal-PEG4-GGFG-Methylene-Compound 147 diluted in DMA was added. Final content of DMA in the reaction system was 10% and final molar ratio of Mal-PEG4-GGFG-Methylene-Compound 147 was 7. The reaction mixture was incubated at 4° C. for 1 hour. During the reaction, 50KD Amicon Ultrafilter were rinsed with 20 mM L-histidine pH5.5. Amicon Ultrafilter was used for the buffer exchange and purification of the final product. The purified product was filtered with 0.22 um filter. Drug-to-antibody ratio (DAR) was determined by liquid chromatography mass spectrometry analysis. Conjugate 47 had a DAR of 4.00.

Other conjugates were synthesized in similar ways as in Examples 69-97 except for using different reacting materials.

### Example 121

### Assessment of Antibody Drug Conjugate Efficacy In Vitro

The antibody drug conjugates of Formula (I) were assayed to evaluate the induction of TNF-α production in tumor cell and Balb/c mouse BMDM (Bone Marrow Derived Macrophages).

### BMDM Co-Culture Assay

The objective of this experiment was to assess the induction of TNF-α production by ADC in tumor cell and Balb/c mouse BMDM (Bone Marrow Derived Macrophages) Co-culture.

Using aseptic technique, bone marrow was extracted from Balb/c mouse tibia and femur bones. After lysis of red blood cells, centrifuge at 450×g for 10 minutes to precipitate white blood cells. Suspended at a concentration of 5×105 cells/mL in growth media (DMEM media supplemented with 10% FBS, 1 mM Sodium Pyruvate, 10 mM HEPES, 1× non-essential amino acids, 50 μg/mL Penicillin, 50 U/mL Streptomycin, 20 ng/mL murine M-CSF) cultured for 7 days to generate macrophages. The following day, BMDM were plated at 8×104 cell/well with tumor cells at 4×104 cell/well in 96-well flat bottom plates in assay media (DMEM media supplemented with 10% FBS). 200 μL of ADC prepared in assay media at a series of diluted concentrations ranging from 0.05 nM to 2.5 μM, thereafter were added to cells in duplicate and incubated for overnight at 37° C. in a 5% CO2 incubator. Prior to supernatant harvest, cells were spun at 350×g for 5 minutes to remove cell debris. TNF-α activity was assessed in the supernatant by ELISA (Elabscience) per the manufacturer's instructions. Optical density at 450 nm (ELISA) was analyzed using a plate reader.

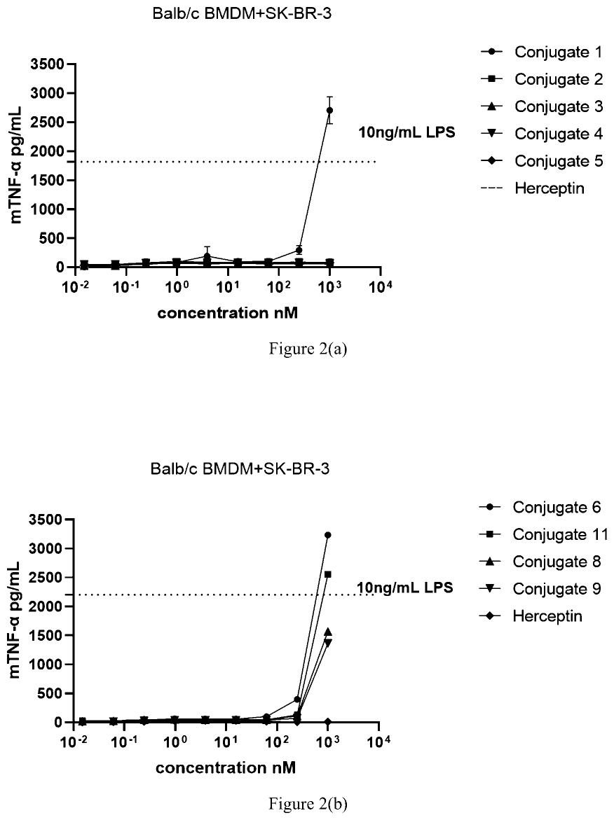

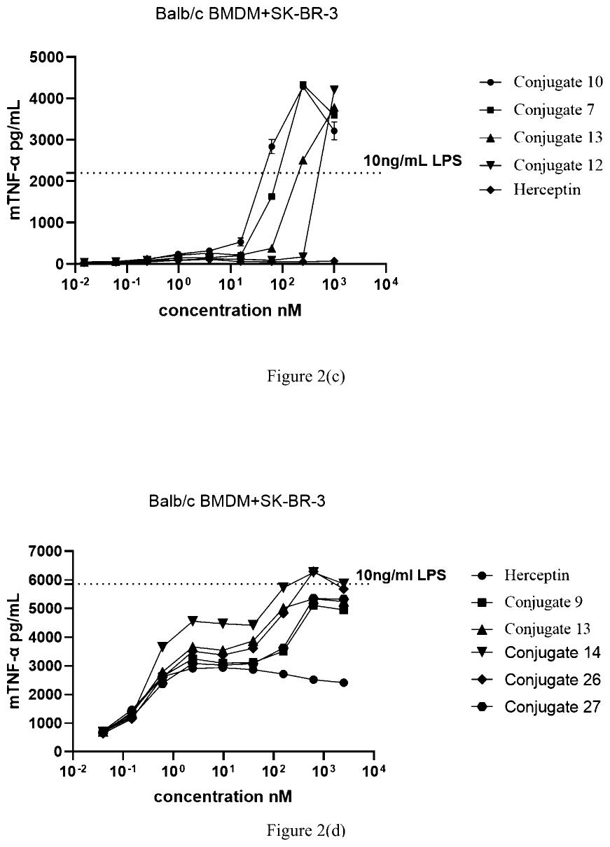

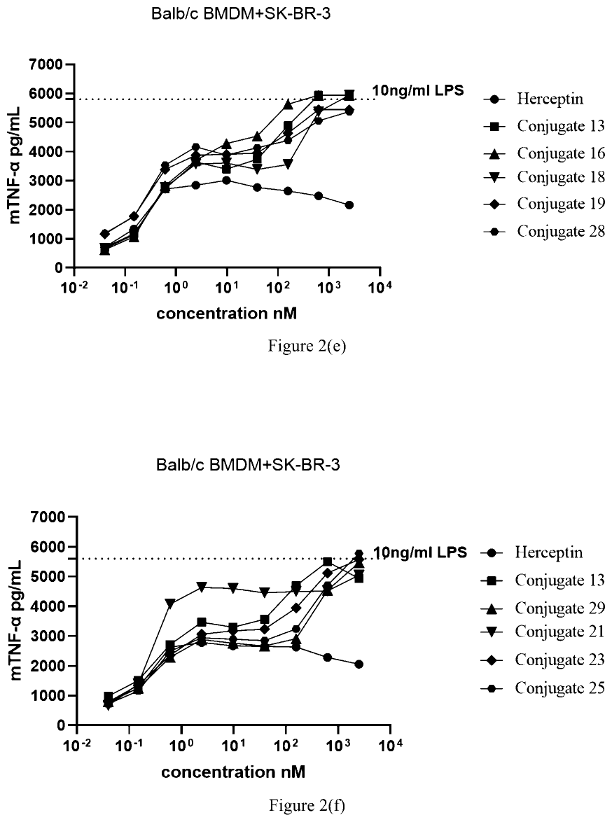

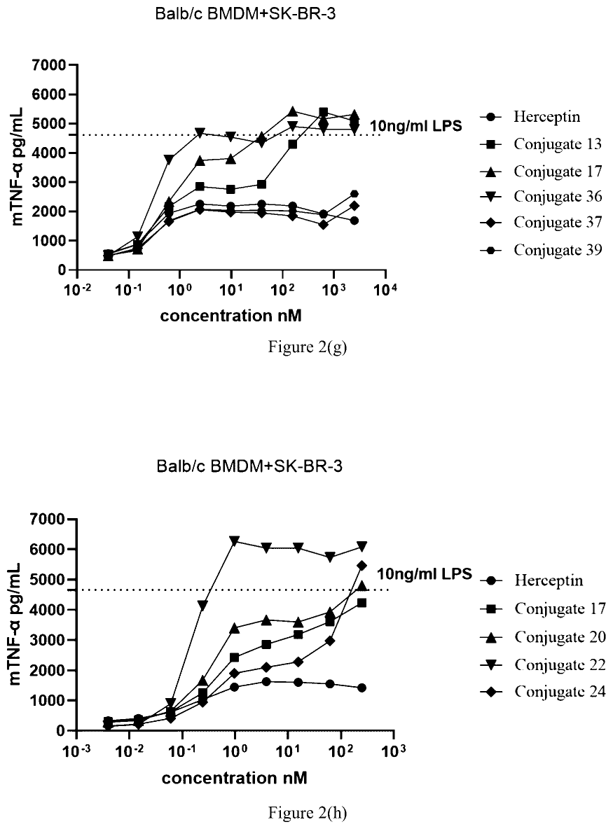

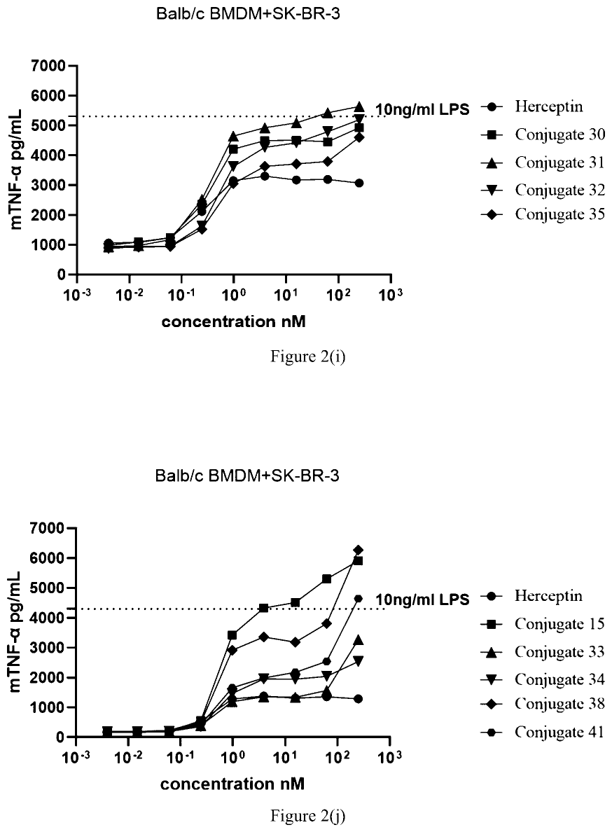

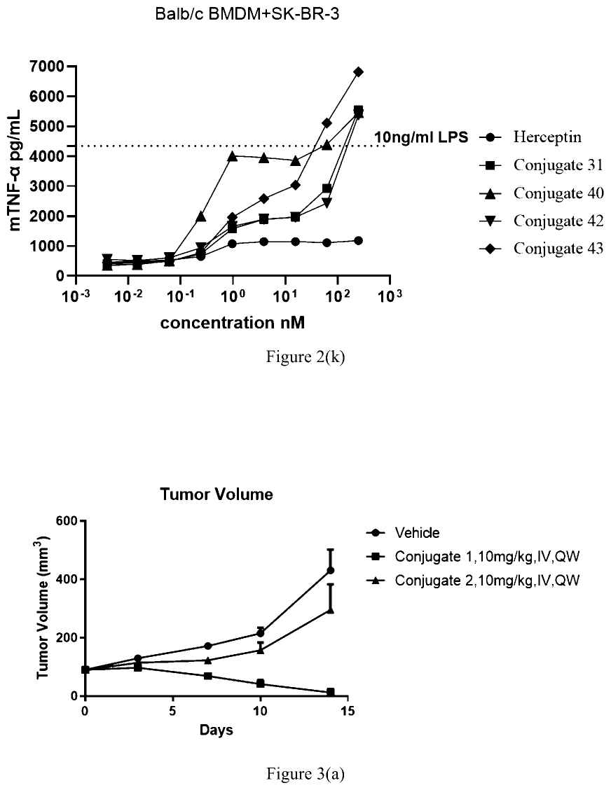

SK-BR-3 cells and Balb/c mice BMDB were co-cultured, and Conjugates 1, 2, 3, 4, 5 and control anti-Her2 antibody (Herceptin) was at indicated concentrations were added. FIG. 2(a) shows Conjugate 1 stimulates higher TNF-α production than control anti-Her2 antibody.

SK-BR-3 cells and Balb/c mice BMDB were co-cultured, and Conjugates 6, 8, 9, 11 and control anti-Her2 antibody (Herceptin) was at indicated concentrations were added. FIG. 2(b) shows Conjugates 6, 8, 9, and 11 stimulate higher TNF-α production than control anti-Her2 antibody.

SK-BR-3 cells and Balb/c mice BMDB were co-cultured, and Conjugates 7, 10, 12, 13 and control anti-Her2 antibody (Herceptin) was at indicated concentrations were added. FIG. 2(c) shows Conjugates 7, 10, 12, and 13 stimulate higher TNF-α production than control anti-Her2 antibody.

SK-BR-3 cells and Balb/c mice BMDB were co-cultured, and Conjugates 9, 13, 14, 26, 27 and control anti-Her2 antibody (Herceptin) was at indicated concentrations were added. FIG. 2(d) shows Conjugates 9, 13, 14, 26, and 27 stimulate higher TNF-α production than control anti-Her2 antibody.

SK-BR-3 cells and Balb/c mice BMDB were co-cultured, and Conjugates 13, 16, 18, 19, 28 and control anti-Her2 antibody (Herceptin) was at indicated concentrations were added. FIG. 2(e) shows Conjugates 13, 16, 18, 19, and 28 stimulate higher TNF-α production than control anti-Her2 antibody.

SK-BR-3 cells and Balb/c mice BMDB were co-cultured, and Conjugates 13, 21, 23, 25, 29 and control anti-Her2 antibody (Herceptin) was at indicated concentrations were added. FIG. 2 (f) shows Conjugates 13, 21, 23, 25, and 29 stimulate higher TNF-α production than control anti-Her2 antibody.

SK-BR-3 cells and Balb/c mice BMDB were co-cultured, and Conjugates 16, 17, 36, 37, 39 and control anti-Her2 antibody (Herceptin) was at indicated concentrations were added. FIG. 2(g) shows Conjugates 13, 17, and 36 stimulate higher TNF-α production than control anti-Her2 antibody.

SK-BR-3 cells and Balb/c mice BMDB were co-cultured, and Conjugates 17, 20, 22, 24 and control anti-Her2 antibody (Herceptin) was at indicated concentrations were added. FIG. 2(h) shows Conjugates 17, 20, 22, and 24 stimulate higher TNF-α production than control anti-Her2 antibody.

SK-BR-3 cells and Balb/c mice BMDB were co-cultured, and Conjugates 30, 31, 32, 35 and control anti-Her2 antibody (Herceptin) was at indicated concentrations were added. FIG. 2(i) shows Conjugates 30, 31, 32, and 35 stimulate higher TNF-α production than control anti-Her2 antibody.

SK-BR-3 cells and Balb/c mice BMDB were co-cultured, and Conjugates 15, 33, 34, 38, 41 and control anti-Her2 antibody (Herceptin) was at indicated concentrations were added. FIG. 2(j) shows Conjugates 15, 33, 34, 38, and 41 stimulate higher TNF-α production than control anti-Her2 antibody.

SK-BR-3 cells and Balb/c mice BMDB were co-cultured, and Conjugates 31, 40, 42, 43 and control anti-Her2 antibody (Herceptin) was at indicated concentrations were added. FIG. 2(k) shows Conjugates 31, 40, 42, and 43 stimulate higher TNF-α production than control anti-Her2 antibody.

### Example 122

### Assessment of Antibody Drug Conjugate Efficacy In Vivo

### Tumor Study 1

BALB/c mice were inoculated subcutaneously in the right lower flank region with EMT6-hHER2 tumor cells (5×10⁵ cells) in 0.1 ml of PBS for tumor development. When the mean tumor size reaches approximately 90 mm³, mice were administered intravenous injections of 10 mpk Conjugates or treated with Vehicle. Subsequent treatments were administered on days 7 and 14 after the initial treatment. After tumor inoculation, the animals were checked daily for morbidity and mortality. During routine monitoring, the animals were checked for any effects of tumor growth and body weight gain/loss (Body weights would be measured every day after randomization).

Tumor volumes were measured twice per week after randomization in two dimensions using a caliper, and the volume was expressed in mm³ using the formula: V=(L×W×W)/2, where V is tumor volume, L is tumor length (the longest tumor dimension) and W is tumor width (the longest tumor dimension perpendicular to L). Dosing as well as tumor and body weight measurements were conducted in a Laminar Flow Cabinet.

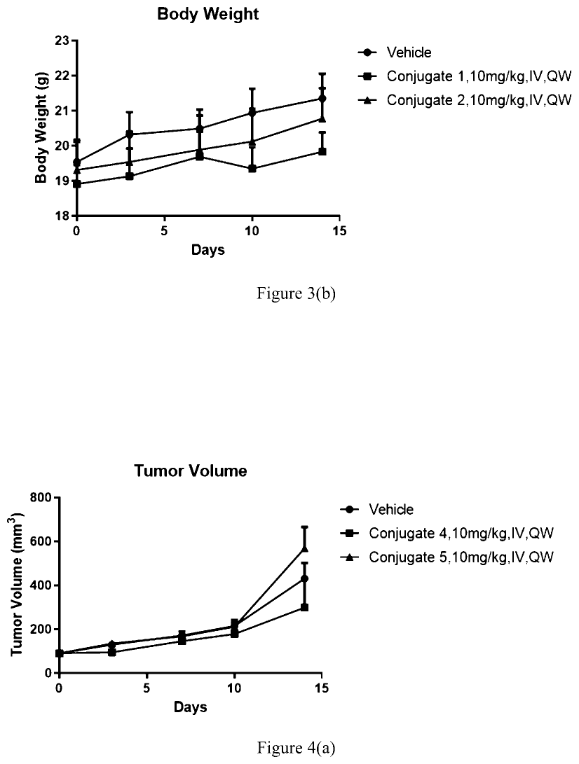

At day 14, Conjugates 1 and 2, 10 mg/kg, i.v., produced significant anti-tumor efficacy against EMT6-hHER2 model in BALB/c mice, with TGI value of 97.1614% with statistically significant difference (P<0.0001) observed compared with vehicle control group (FIGS. 3(a) and 3(b)). No obvious body weight loss was found in mice of treatment groups.

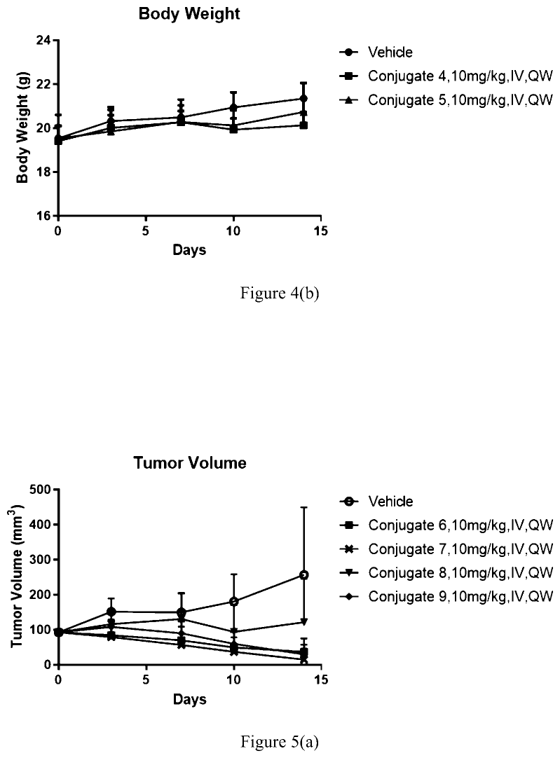

At day 14, Conjugates 4 and 5, 10 mg/kg, i.v., produced significant anti-tumor efficacy against EMT6-hHER2 model in Balb/c mice (P<0.05) compared with vehicle control group (FIGS. 4(a) and 4(b)). No body weight loss was observed in mice threated with Conjugate 4 and 5, 10 mg/kg, i.v., suggests mice were tolerable under these designed treatment regimens.

At day 14, Conjugates 6, 7, 8, and 9, 10 mg/kg, i.v., produced significant anti-tumor efficacy against EMT6-hHER2 model in Balb/c mice (P<0.01) observed compared with vehicle control group (FIGS. 5(a) and 5(b)). No obviously body weight loss was observed in mice threated with Conjugate 6, 7, 8, and 9, 10 mg/kg, i.v., suggests mice were tolerable under these designed treatment regimens.

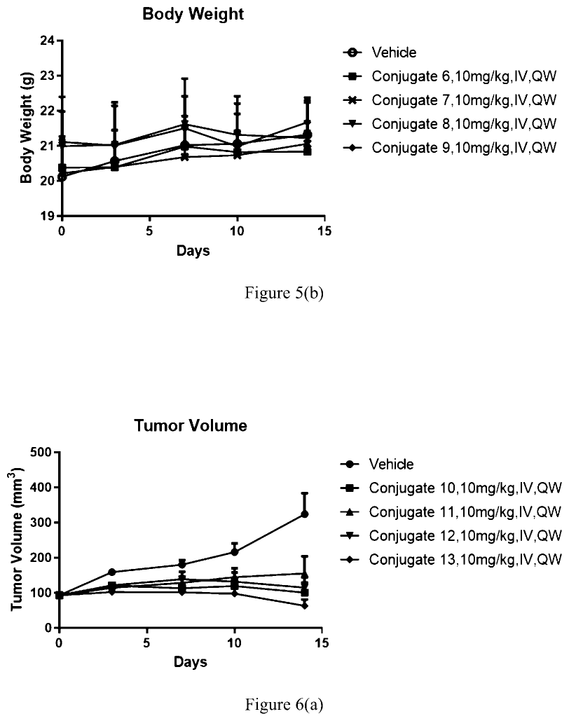

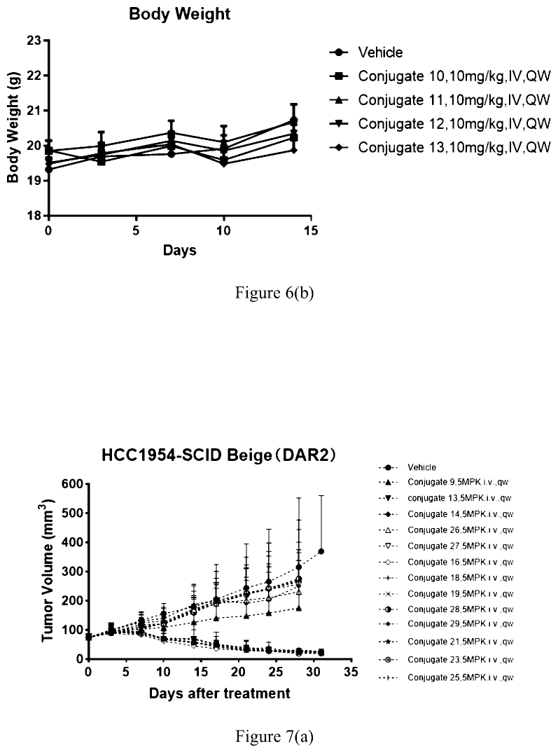

At day 14, Conjugate 10, 11, 12, and 13, 10 mg/kg, i.v., produced significant anti-tumor efficacy against EMT6-hHER2 model in Balb/c mice (P<0.01) observed compared with vehicle control group (FIGS. 6(a) and 6(b)). No obviously body weight loss was observed in mice threated with Conjugate 10, 11, 12, and 13, 10 mg/kg, i.v., suggests mice were tolerable under these designed treatment regimens.

### Tumor Study 2

SCID Beige mice were inoculated subcutaneously in the right lower flank region with HCC1954 tumor cells (5×106 cells with matrigel) for tumor development. When the mean tumor size reaches approximately 74 mm³, mice were administered intravenous injections of 5 mpk Conjugates or treated with Vehicle. Subsequent treatments were administered on days 7/14/21 after the initial treatment. After tumor inoculation, the animals were checked daily for morbidity and mortality. During routine monitoring, the animals were checked for any effects of tumor growth and body weight gain/loss (Body weights would be measured every day after randomization).

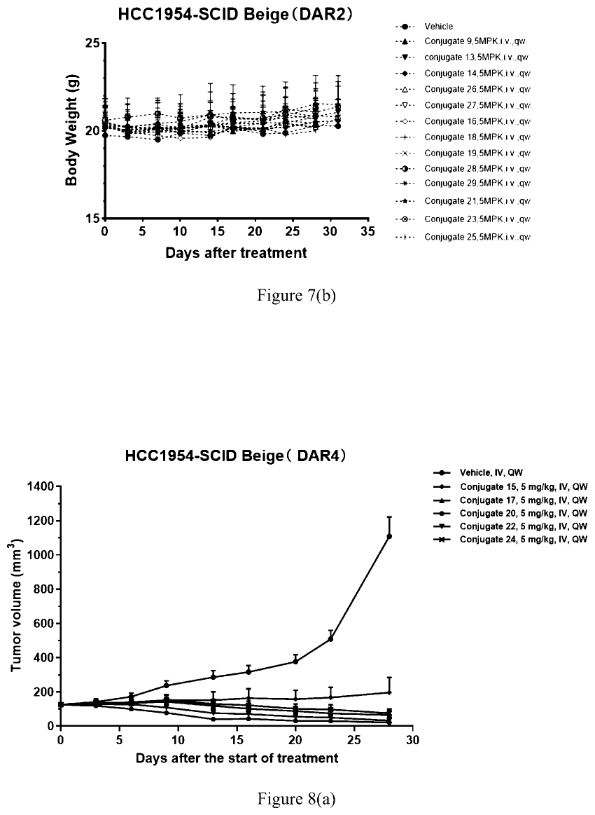

At day 21, Conjugate 9, 13, 14, 16, 18, 19, 21, 23, 25, 26, 27, 28, and 29, 5 mg/kg, i.v., produced significant anti-tumor efficacy against HCC1954 model in SCID mice compared with vehicle control group (FIGS. 7(a) and 7(b)). No body weight loss was observed in mice threated with Conjugate 9, 13, 14, 16, 18, 19, 21, 23, 25, 26, 27, 28, and 29, 5 mg/kg, i.v., suggests mice were tolerable under these designed treatment regimens.

### Tumor Study 3

SCID Beige mice were inoculated subcutaneously in the right lower flank region with HCC1954 tumor cells (5×106 cells with matrigel) for tumor development. When the mean tumor size reaches approximately 126 mm³, mice were administered intravenous injections of 5 mpk Conjugates or treated with Vehicle. Subsequent treatments were administered on days 7/14/21 after the initial treatment. After tumor inoculation, the animals were checked daily for morbidity and mortality. During routine monitoring, the animals were checked for any effects of tumor growth and body weight gain/loss (Body weights would be measured every day after randomization).

At day 21, Conjugate 15, 17, 20, 22, and 24, 5 mg/kg, i.v., produced significant anti-tumor efficacy against HCC1954 model in SCID mice compared with vehicle control group (FIGS. 8(a) and 8(b)). No body weight loss was observed in mice threated with Conjugate 15, 17, 20, 22, and 24, 5 mg/kg, i.v., suggests mice were tolerable under these designed treatment regimens.

### Tumor Study 4

SCID Beige mice were inoculated subcutaneously in the right lower flank region with HCC1954 tumor cells (5×106 cells with matrigel) for tumor development. When the mean tumor size reaches approximately 126 mm³, mice were administered intravenous injections of 2.5 mpk Conjugates or treated with Vehicle. Subsequent treatments were administered on days 7/14/21 after the initial treatment. After tumor inoculation, the animals were checked daily for morbidity and mortality. During routine monitoring, the animals were checked for any effects of tumor growth and body weight gain/loss (Body weights would be measured every day after randomization).

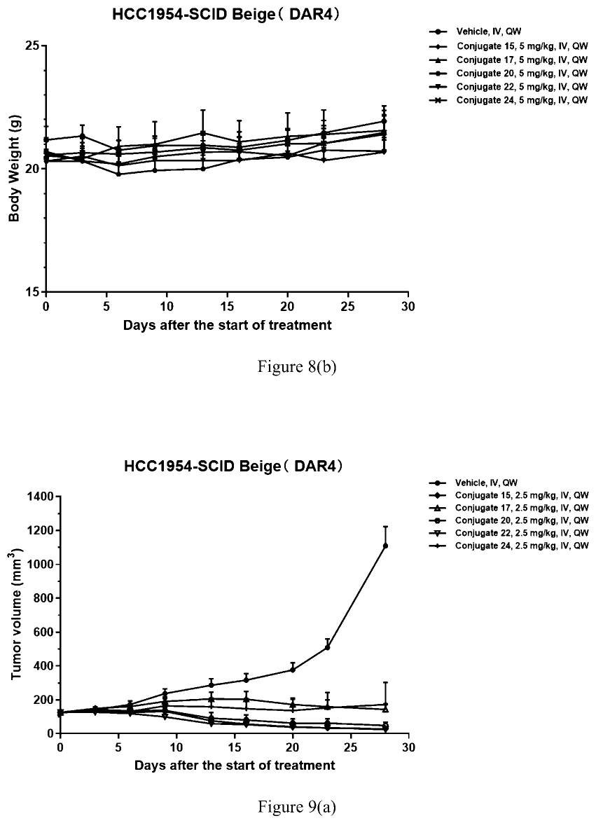

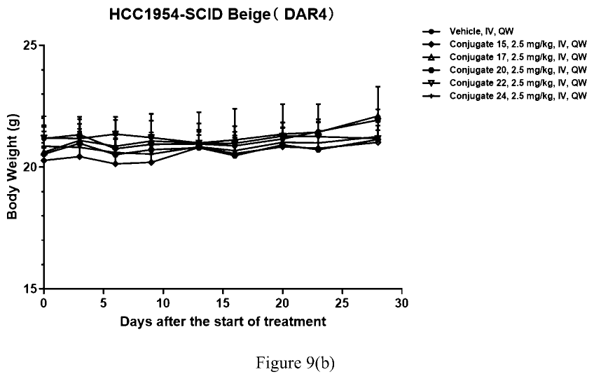

At day 21, Conjugate 15, 17, 20, 22, and 24, 2.5 mg/kg, i.v., produced significant anti-tumor efficacy against HCC1954 model in SCID mice compared with vehicle control group (FIGS. 9(a) and 9(b)). No body weight loss was observed in mice threated with Conjugate 15, 17, 20, 22, and 24, 2.5 mg/kg, i.v., suggests mice were tolerable under these designed treatment regimens.

All the conjugates provided herein produced significant anti-tumor efficacy against HCC1954 in SCID mice model and are tolerable for mice, compared with vehicle group.

The foregoing description is considered as illustrative only of the principles of the present disclosure. Further, since numerous modifications and changes will be readily apparent to those skilled in the art, it is not desired to limit the invention to the exact construction and process shown as described above. Accordingly, all suitable modifications and equivalents may be considered to fall within the scope of the invention as defined by the claims that follow.

The words “comprise”, “comprising”, “include”, “including”, and “includes” when used in this specification and in the following claims are intended to specify the presence of stated features, integers, components, or steps, but they do not preclude the presence or addition of one or more other features, integers, components, steps, or groups thereof.

1.-201. (canceled)

202. A conjugate compound having Formula (I): 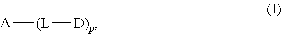
or a pharmaceutically acceptable salt thereof, wherein
A is a targeting moiety;
L is a linker;
p is an integer from 1 to 8;
D is a payload unit of Formula (II): 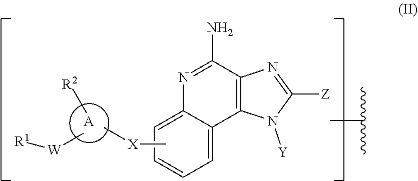
wherein:
X is selected from the group consisting of —O—, —S—, —NH—, —(CH₂)ᵢ—, —(X¹)NC(O)—, —(X¹)NS(O)₂—, —C(O)N(X¹)— and —S(O)₂N(X¹)—, wherein —NH— and —(CH₂)ᵢ— are optionally substituted with one or more groups independently selected from halogen, hydroxyl, cyano, amino or alkyl;
each X¹ is independently hydrogen, alkyl, alkenyl, or haloalkyl;
Ring A is cycloalkyl, heterocyclyl, aryl or heteroaryl;
W is selected from the group consisting of a direct bond, alkyl, cycloalkyl, heterocyclyl, *—W¹-alkyl-, *-alkyl-W¹—, and *-alkyl-W¹-alkyl-, wherein the alkyl, cycloalkyl, heterocyclyl, and alkyl in *—W¹-alkyl-, *-alkyl-W¹—, and *-alkyl-W¹-alkyl are optionally substituted with one or more groups independently selected from halogen, hydroxyl, cyano, amino, alkyl and —C(O)OR^{a}, and wherein * end of W is connected to Ring A;
W¹ is —O—, —NR^{a}—, —C(O)—, —C(O)NR^{a}—, —NR^{a}C(O)—, —OC(O)NR^{a}—, —NR^{a}C(O)O—, or —NR^{a}C(O)NR^{a}—,
each R^{a} is independently hydrogen, alkyl, or haloalkyl;
R¹ is hydrogen, —N(R^{b})₂, hydroxyl or —SH;
each R^{b} is independently hydrogen, alkyl, or haloalkyl, or
two R^{b} taken together with the nitrogen atom to which they are bound form a heterocyclyl;
R² is hydrogen, halogen, hydroxyl, cyano, amino, alkyl, alkenyl, alkynyl, alkoxyl, haloalkyl, —OR^{e}, —OC(O)R^{e}, —SR^{e}, —B(OH)₂, —NO₂, —CHO, —C(═NOR^{e})(R^{e}), —R²⁻¹—N(R^{e})₂, —R²⁻¹—N(R^{e})C(O)R^{e}, —R²⁻¹—N(R^{e})S(O)₂R^{e}, —R²⁻¹—N(R^{e})P(O)₂R^{e}, —R²⁻¹—C(O)OR^{e}, —R²⁻¹—C(O)N(R^{e})₂, —R²⁻¹—S(O)₂N(R^{e})₂, —R²⁻¹—P(O)₂N(R^{e})₂, —OC(O)NR^{e}, or —NC(O)NR^{e};
each R²⁻¹ is independently null or alkyl;
each R^{e} is independently hydrogen, alkyl, or haloalkyl;
Y is —Y¹—Y²—Y³, wherein
Y¹ is a direct bond or —(CH₂)ₘ-Q¹-(CH₂)ₙ—O—*, wherein —(CH₂)ₘ— and —(CH₂)ₙ— are optionally substituted with one or more groups independently selected from halogen, hydroxyl, alkyl or haloalkyl, and * end of Y¹ is connected to Y²;
Y² is a direct bond or —(CH₂)ₛ-Q²-(CH₂)ₜ—NR^{c}—**, wherein —(CH₂)ₛ— and —(CH₂)ₜ— are optionally substituted with one or more groups independently selected from halogen, hydroxyl, alkyl or haloalkyl, and ** end of Y² is connect to Y³;
Y³ is selected from the group consisting of hydrogen, alkyl, -alkyl-aryl, -alkyl-N(R^{b})₂, —C(O)-alkyl, —C(O)-alkyl-N(R^{b})₂, and —S(O)₂-alkyl;
Q¹ and Q² are each independently selected from a direct bond, cycloalkyl, heterocyclyl, aryl, or heteroaryl, wherein the cycloalkyl, heterocyclyl, aryl, and heteroaryl are optionally substituted with one or more groups independently selected from halogen, hydroxyl, cyano, amino, or alkyl;
R^{c} is hydrogen or alkyl, or
R^{c} and Y³ taken together with the atoms to which they are bound form a heterocyclyl optionally substituted with one or more groups independently selected from halogen, hydroxyl, cyano, amino or alkyl;
Z is selected from the group consisting of alkyl, alkenyl, alkynyl, heteroalkyl, heteroalkenyl and heteroalkynyl, each of which is optionally substituted with one or more R^{d};
R^{d} is selected from the group consisting of halogen, acyl, alkyl, alkenyl, alkynyl, cycloalkyl and —O-cycloalkyl;
i is 0, 1, 2, 3, 4, 5 or 6;
m is 0, 1,2, 3, 4 or 5;
n is 0, 1, 2, 3, 4 or 5;
s is 0, 1, 2, 3, 4 or 5; and
t is 0, 1, 2, 3, 4 or 5.

203. The conjugate compound of claim 202, wherein ring A is selected from the group consisting of:

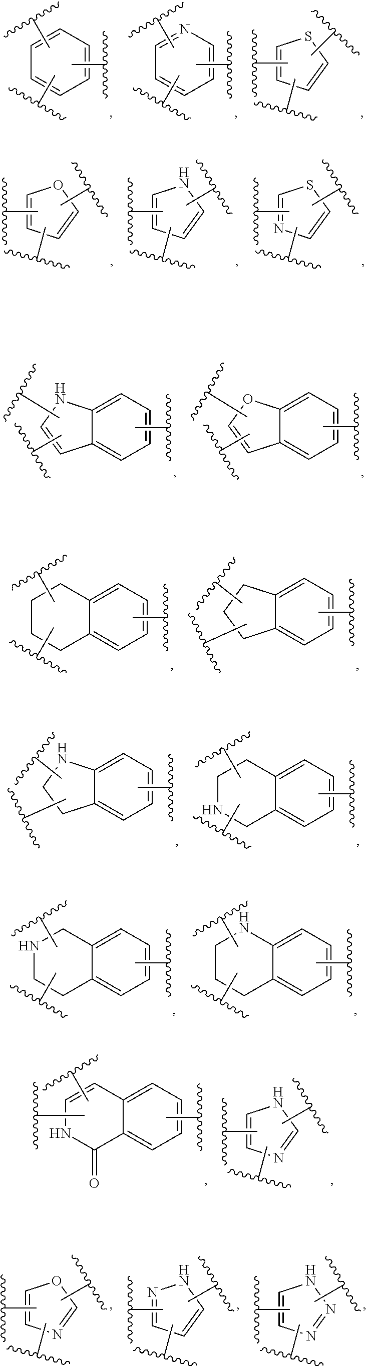

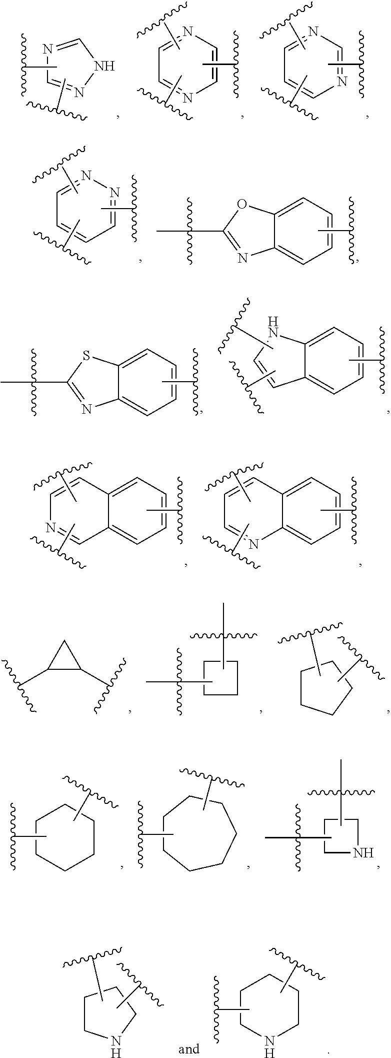

204. The conjugate compound of claim 202, wherein X is —O—, —NH—, or —(CH₂)ᵢ—, wherein —NH— and —(CH₂)ᵢ— are optionally substituted with one or more halogen or alkyl.

205. The conjugate compound of claim 202, wherein R¹ is hydrogen, —N(R^{b})₂ or hydroxyl.

206. The conjugate compound of claim 202, wherein W is a direct bond, and/or R¹ is hydrogen, —N(R)₂ or hydroxyl, and each R is independently hydrogen or alkyl, or two R taken together with the nitrogen atom to which they are bound form a heterocyclyl.

207. The conjugate compound of claim 202, wherein:
i. W is alkyl optionally substituted with —C(O)OR^{a}, and/or R¹ is hydrogen, hydroxyl or —N(R^{b})₂, and each R is independently hydrogen or alkyl, or two R taken together with the nitrogen atom to which they are bound form a heterocyclyl; or
ii. W is *—W¹-alkyl- and/or W¹ is —O—, —NR^{a}—, —C(O)NR^{a}—, —OC(O)NR^{a}—, —NR^{a}C(O)—, —NR^{a}C(O)O—, or —NR^{a}C(O)NR^{a}—, and each R^{a} is independently hydrogen, alkyl, or haloalkyl, and/or R¹ is —NH₂ or —NH—CH₃; or
iii. W is *-alkyl-W¹—, and/or W¹ is —C(O)— and/or R¹ is —NH₂; or
iv. W is *-alkyl-W¹-alkyl-, and/or W¹ is —NR^{a}C(O)— or —OC(O)NR^{a}—, and R^{a} is hydrogen, alkyl, or haloalkyl, and/or R¹ is hydroxyl, —NH₂ or —NH—CH₃; or
v. W is cycloalkyl, and/or R¹ is —NH₂; or
vi. W is heterocyclyl, and/or R¹ is hydrogen.

208. The conjugate compound of claim 202, wherein R² is hydrogen, halogen, cyano, alkyl or alkoxyl; and/or.

209. The conjugate compound of claim 202, wherein
i. Y¹ is a direct bond, and/or Y² is a direct bond, and/or Y³ is -alkyl-aryl; or
ii. Y¹ is a direct bond, and/or Y² is —(CH₂)ₛ-Q²-(CH₂)ₜ—NR^{c}—**, and/or Q² is a direct bond, cycloalkyl or aryl, wherein the cycloalkyl or aryl is optionally substituted with one or more groups independently selected from halogen or alkyl, and/or R^{c} is hydrogen, and/or Y³ is hydrogen, -alkyl-N(R^{b})₂, or —S(O)₂-alkyl; or
iii. Y¹ is —(CH₂)ₘ-Q¹-(CH₂)ₙ—O—*, and/or Q¹ is a direct bond, cycloalkyl or aryl, wherein the cycloalkyl or aryl is optionally substituted with one or more groups independently selected from halogen or alkyl, and/or Y² is a direct bond, and/or Y³ is hydrogen or alkyl; or
iv. Y¹ is —(CH₂)ₘ-Q¹-(CH₂)ₙ—O—*, and/or Y² is —(CH₂)ₛ-Q²-(CH₂)ₜ—NR^{c}—**, and/or Q² is a direct bond, and/or R^{c} is hydrogen, and/or Y³ is hydrogen, —C(O)-alkyl or —C(O)-alkyl-N(R^{b})₂, or R^{c} and Y³ taken together with the atoms to which they are bound form a heterocyclyl optionally substituted with one or more halogen.

210. The conjugate compound of claim 202, wherein Z is selected from the group consisting of alkyl, alkenyl, alkynyl and heteroalkyl, which is optionally substituted with one or more R^{d}, optionally R^{d} is selected from the group consisting of halogen, acyl, alkyl, cycloalkyl and —O-cycloalkyl.

211. The conjugate compound of claim 202, wherein D is a payload unit of Formula (IIa): 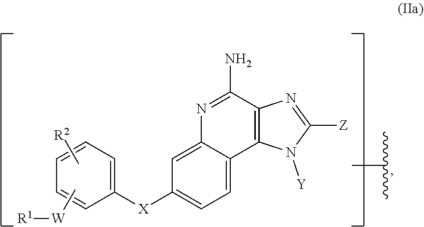
optionally the payload unit is selected from the group consisting of:

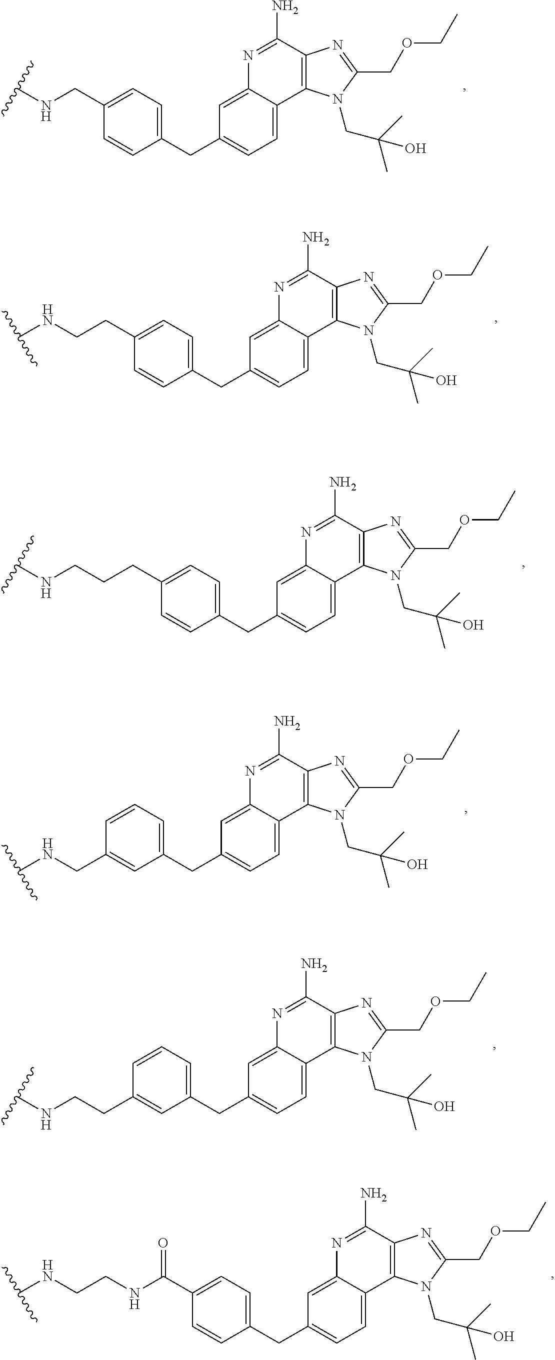 [SMILES: *NCc1ccc(Cc2ccc3c(c2)nc(N)c2nc(COCC)n(CC(C)(C)O)c23)cc1 ; *NCCc1ccc(Cc2ccc3c(c2)nc(N)c2nc(COCC)n(CC(C)(C)O)c23)cc1 ; *NCCCc1ccc(Cc2ccc3c(c2)nc(N)c2nc(COCC)n(CC(C)(C)O)c23)cc1 ; *NCc1cccc(Cc2ccc3c(c2)nc(N)c2nc(COCC)n(CC(C)(C)O)c23)c1 ; *NCCc1cccc(Cc2ccc3c(c2)nc(N)c2nc(COCC)n(CC(C)(C)O)c23)c1 ; *NCCNC(=O)c1ccc(Cc2ccc3c(c2)nc(N)c2nc(COCC)n(CC(C)(C)O)c23)cc1]

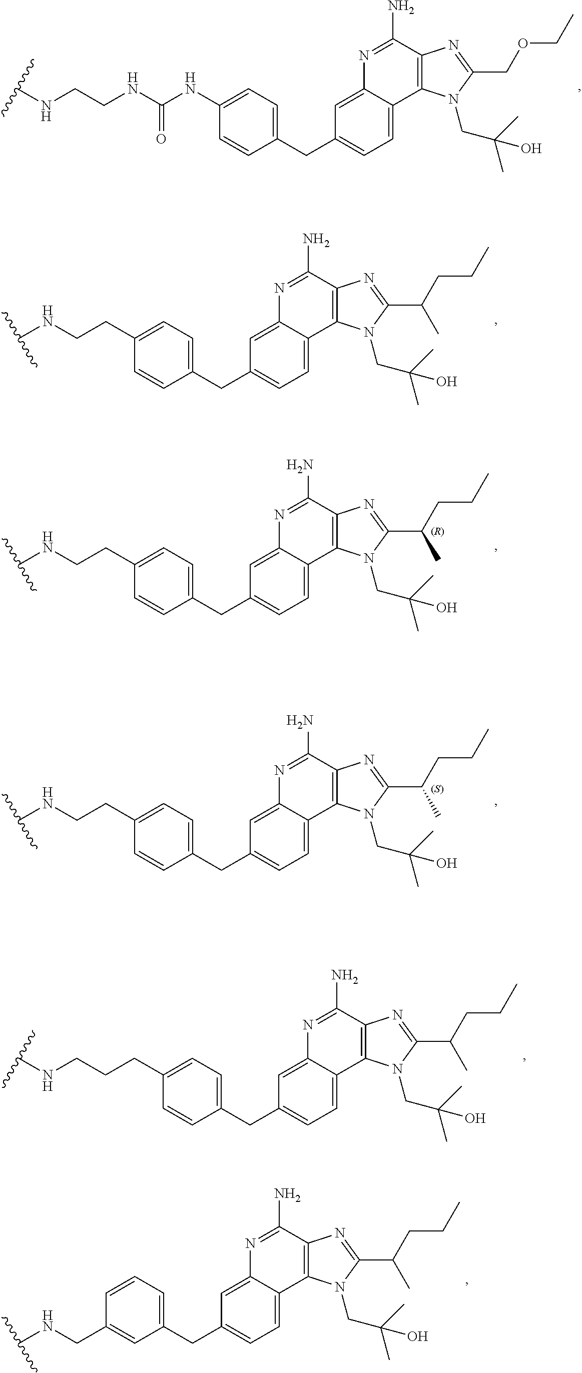

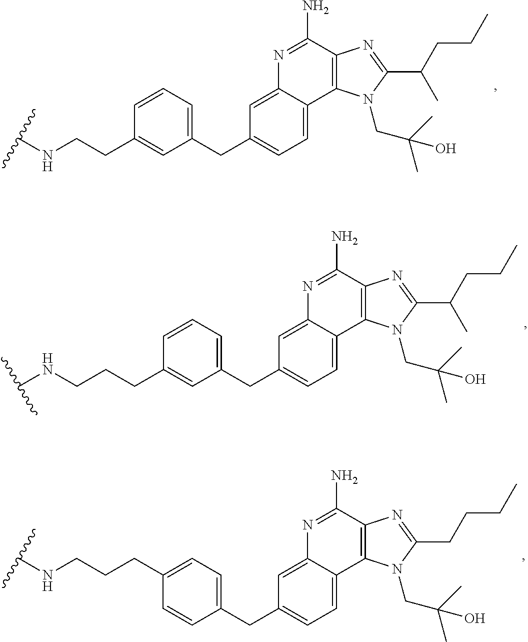 [SMILES: *NCCc1cccc(Cc2ccc3c(c2)nc(N)c2nc(C(C)CCC)n(CC(C)(C)O)c23)c1 ; *NCCCc1cccc(Cc2ccc3c(c2)nc(N)c2nc(C(C)CCC)n(CC(C)(C)O)c23)c1 ; *NCCCc1ccc(Cc2ccc3c(c2)nc(N)c2nc(CCCC)n(CC(C)(C)O)c23)cc1]

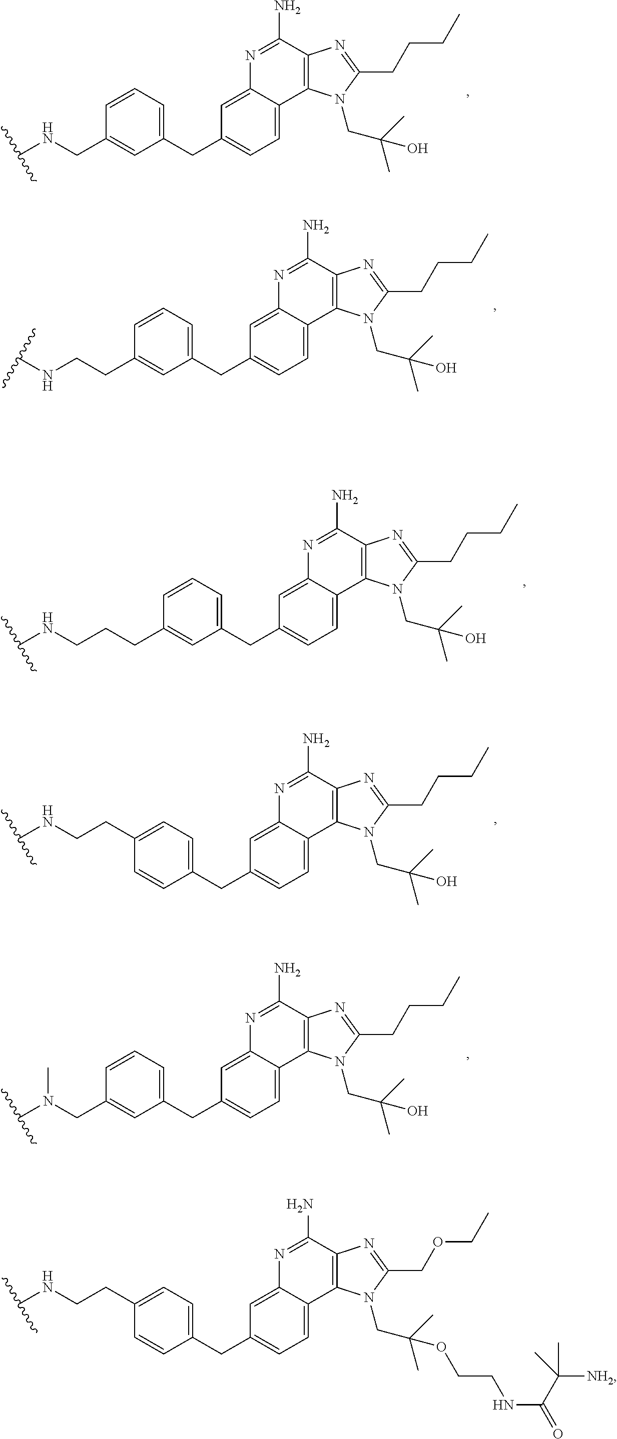 [SMILES: *NCc1cccc(Cc2ccc3c(c2)nc(N)c2nc(CCCC)n(CC(C)(C)O)c23)c1 ; *NCCc1cccc(Cc2ccc3c(c2)nc(N)c2nc(CCCC)n(CC(C)(C)O)c23)c1 ; *NCCCc1cccc(Cc2ccc3c(c2)nc(N)c2nc(CCCC)n(CC(C)(C)O)c23)c1 ; *NCCc1ccc(Cc2ccc3c(c2)nc(N)c2nc(CCCC)n(CC(C)(C)O)c23)cc1 ; *N(C)Cc1cccc(Cc2ccc3c(c2)nc(N)c2nc(CCCC)n(CC(C)(C)O)c23)c1 ; *NCCc1ccc(Cc2ccc3c(c2)nc(N)c2nc(COCC)n(CC(C)(C)OCCNC(=O)C(C)(C)N)c23)cc1]

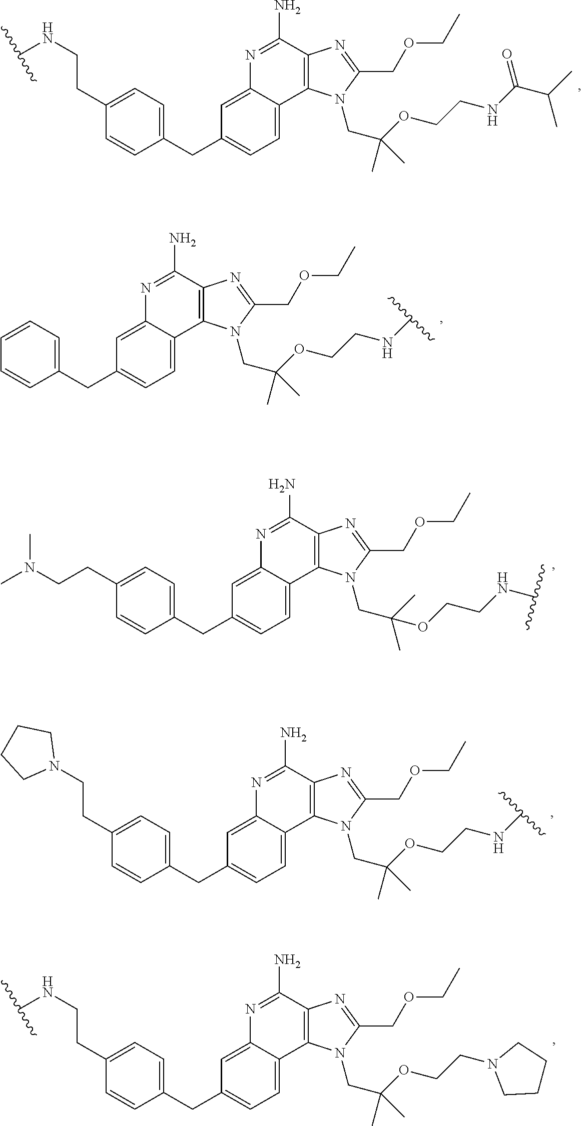

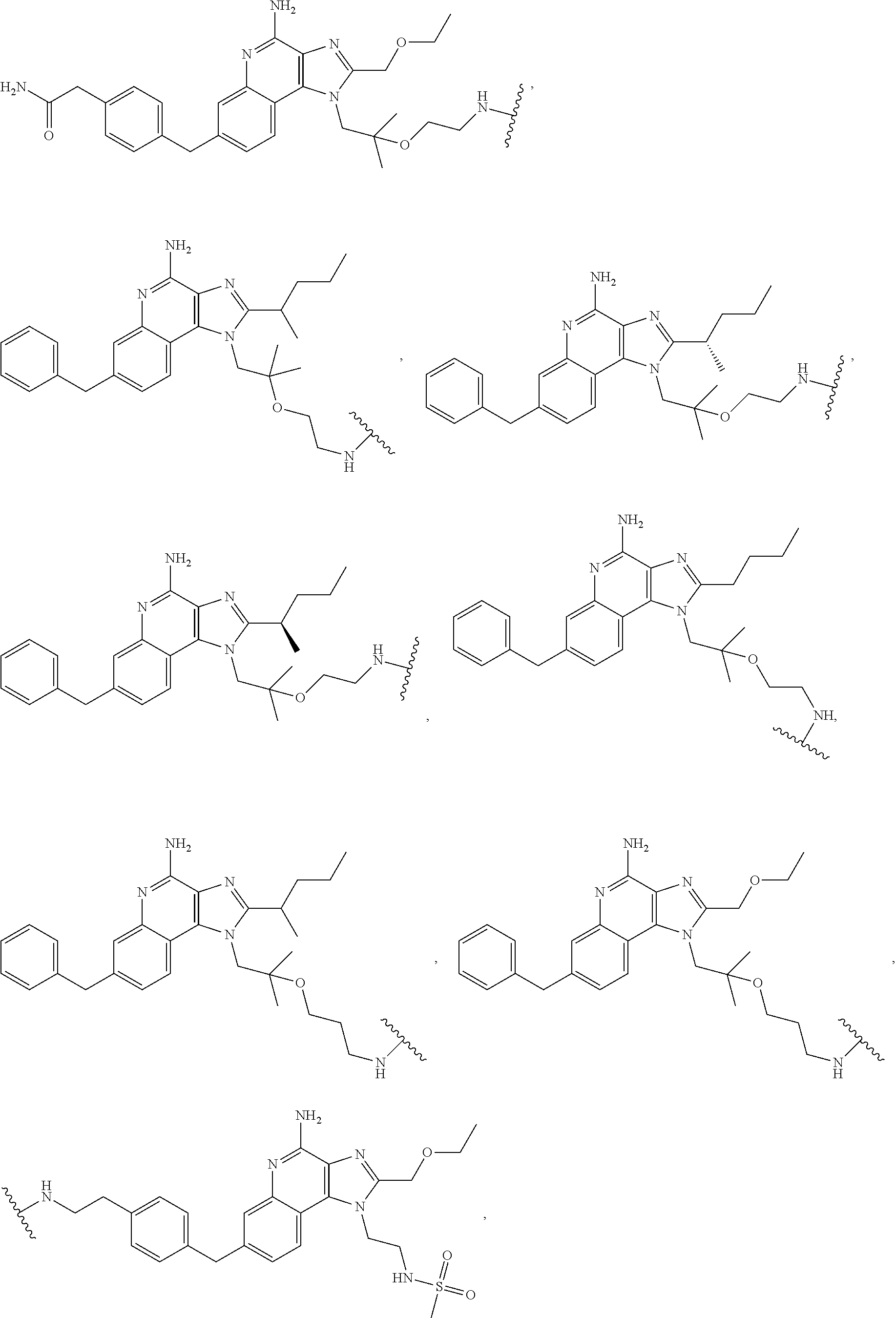

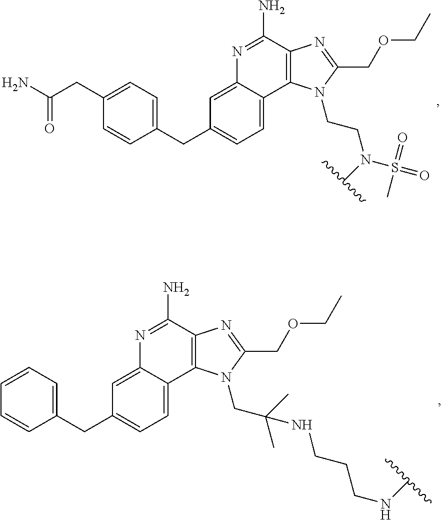 [SMILES: *N(CCn1c(COCC)nc2c(N)nc3cc(Cc4ccc(CC(N)=O)cc4)ccc3c21)S(C)(=O)=O ; *NCCCNC(C)(C)Cn1c(COCC)nc2c(N)nc3cc(Cc4ccccc4)ccc3c21]

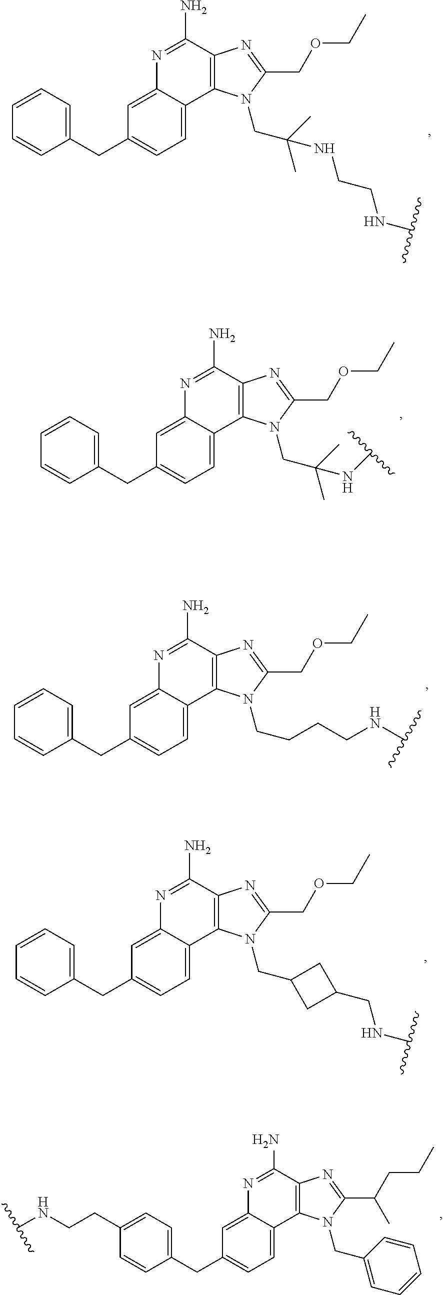 [SMILES: *NCCNC(C)(C)Cn1c(COCC)nc2c(N)nc3cc(Cc4ccccc4)ccc3c21 ; *NC(C)(C)Cn1c(COCC)nc2c(N)nc3cc(Cc4ccccc4)ccc3c21 ; *NCCCCn1c(COCC)nc2c(N)nc3cc(Cc4ccccc4)ccc3c21 ; *NCC1CC(Cn2c(COCC)nc3c(N)nc4cc(Cc5ccccc5)ccc4c32)C1 ; *NCCc1ccc(Cc2ccc3c(c2)nc(N)c2nc(C(C)CCC)n(Cc4ccccc4)c23)cc1]

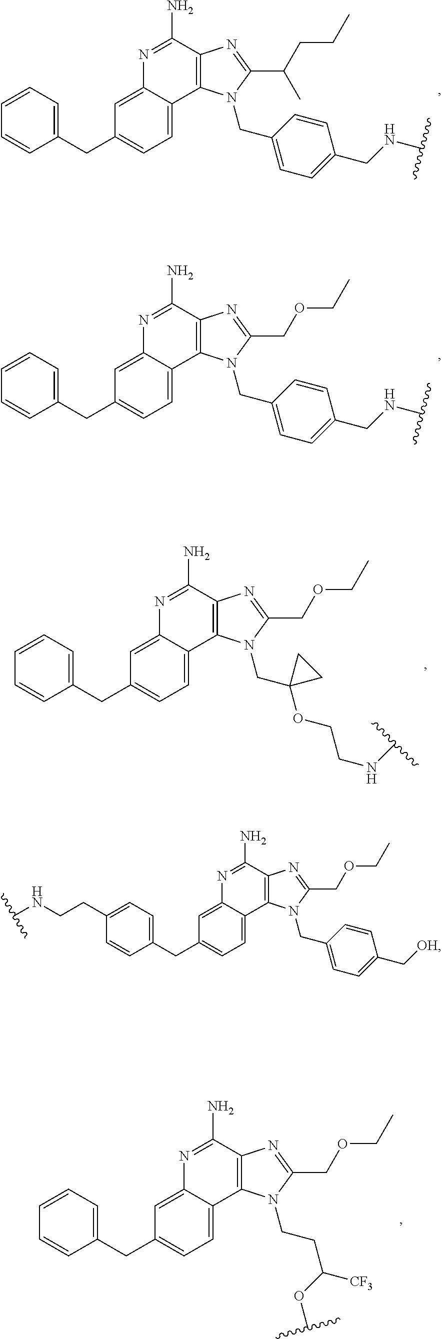 [SMILES: *NCc1ccc(Cn2c(C(C)CCC)nc3c(N)nc4cc(Cc5ccccc5)ccc4c32)cc1 ; *NCc1ccc(Cn2c(COCC)nc3c(N)nc4cc(Cc5ccccc5)ccc4c32)cc1 ; *NCCOC1(Cn2c(COCC)nc3c(N)nc4cc(Cc5ccccc5)ccc4c32)CC1 ; *NCCc1ccc(Cc2ccc3c(c2)nc(N)c2nc(COCC)n(Cc4ccc(CO)cc4)c23)cc1 ; *OC(CCn1c(COCC)nc2c(N)nc3cc(Cc4ccccc4)ccc3c21)C(F)(F)F]

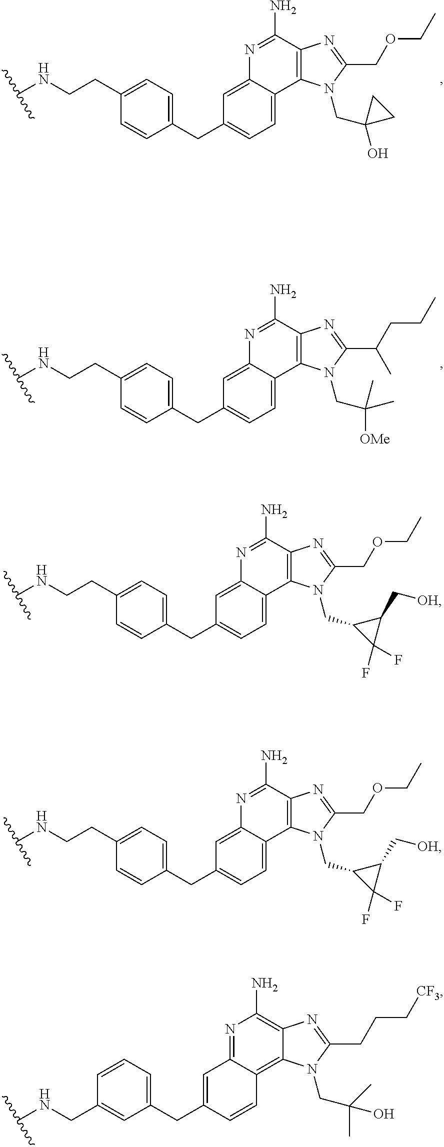 [SMILES: *NCCc1ccc(Cc2ccc3c(c2)nc(N)c2nc(COCC)n(CC4(O)CC4)c23)cc1 ; *NCCc1ccc(Cc2ccc3c(c2)nc(N)c2nc(C(C)CCC)n(CC(C)(C)OC)c23)cc1 ; *NCCc1ccc(Cc2ccc3c(c2)nc(N)c2nc(COCC)n(C[C@@H]4[C@@H](CO)C4(F)F)c23)cc1 ; *NCCc1ccc(Cc2ccc3c(c2)nc(N)c2nc(COCC)n(C[C@@H]4[C@H](CO)C4(F)F)c23)cc1 ; *NCc1cccc(Cc2ccc3c(c2)nc(N)c2nc(CCCC(F)(F)F)n(CC(C)(C)O)c23)c1]

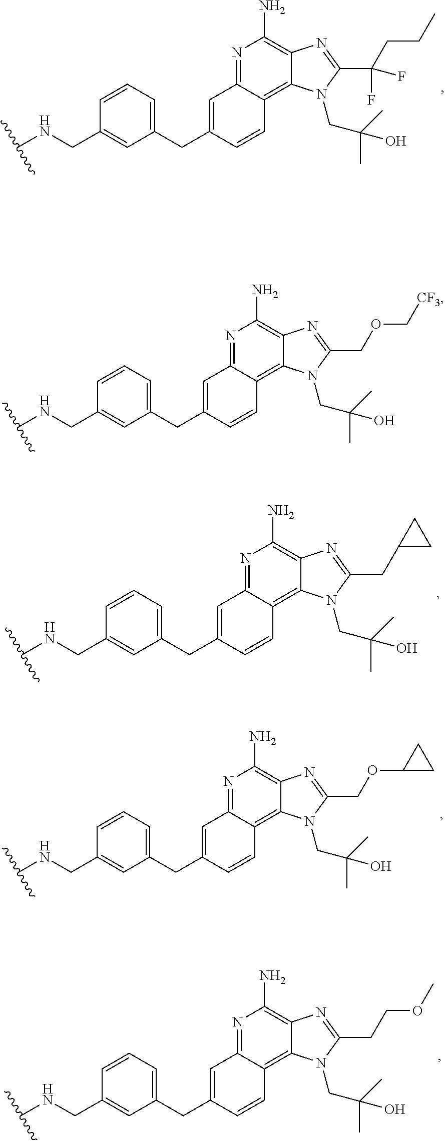 [SMILES: *NCc1cccc(Cc2ccc3c(c2)nc(N)c2nc(C(F)(F)CCC)n(CC(C)(C)O)c23)c1 ; *NCc1cccc(Cc2ccc3c(c2)nc(N)c2nc(COCC(F)(F)F)n(CC(C)(C)O)c23)c1 ; *NCc1cccc(Cc2ccc3c(c2)nc(N)c2nc(CC4CC4)n(CC(C)(C)O)c23)c1 ; *NCc1cccc(Cc2ccc3c(c2)nc(N)c2nc(COC4CC4)n(CC(C)(C)O)c23)c1 ; *NCc1cccc(Cc2ccc3c(c2)nc(N)c2nc(CCOC)n(CC(C)(C)O)c23)c1]

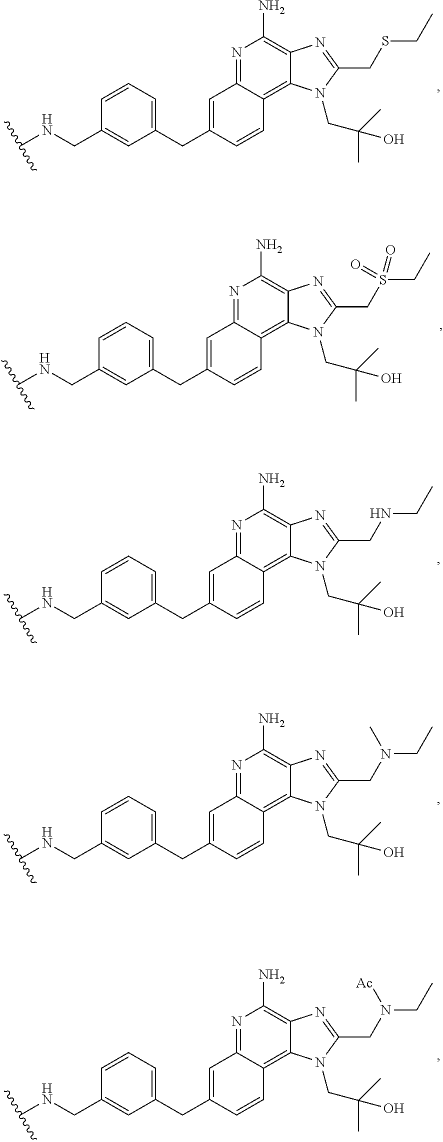 [SMILES: *NCc1cccc(Cc2ccc3c(c2)nc(N)c2nc(CSCC)n(CC(C)(C)O)c23)c1 ; *NCc1cccc(Cc2ccc3c(c2)nc(N)c2nc(CS(=O)(=O)CC)n(CC(C)(C)O)c23)c1 ; *NCc1cccc(Cc2ccc3c(c2)nc(N)c2nc(CNCC)n(CC(C)(C)O)c23)c1 ; *NCc1cccc(Cc2ccc3c(c2)nc(N)c2nc(CN(C)CC)n(CC(C)(C)O)c23)c1 ; *NCc1cccc(Cc2ccc3c(c2)nc(N)c2nc(CN(CC)C(C)=O)n(CC(C)(C)O)c23)c1]

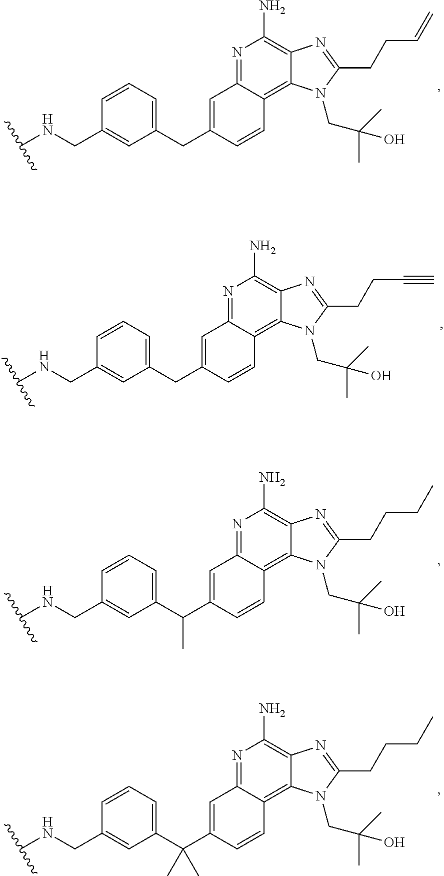 [SMILES: *NCc1cccc(Cc2ccc3c(c2)nc(N)c2nc(CCC=C)n(CC(C)(C)O)c23)c1 ; *NCc1cccc(Cc2ccc3c(c2)nc(N)c2nc(CCC#C)n(CC(C)(C)O)c23)c1 ; *NCc1cccc(C(C)c2ccc3c(c2)nc(N)c2nc(CCCC)n(CC(C)(C)O)c23)c1 ; *NCc1cccc(C(C)(C)c2ccc3c(c2)nc(N)c2nc(CCCC)n(CC(C)(C)O)c23)c1]

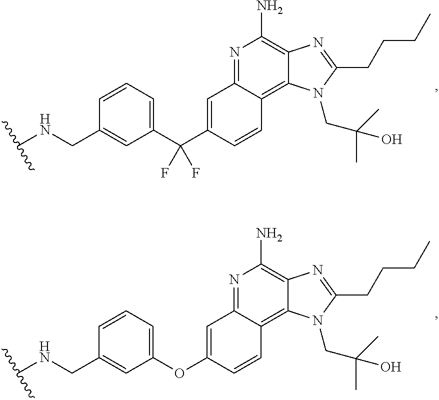 [SMILES: *NCc1cccc(C(F)(F)c2ccc3c(c2)nc(N)c2nc(CCCC)n(CC(C)(C)O)c23)c1 ; *NCc1cccc(Oc2ccc3c(c2)nc(N)c2nc(CCCC)n(CC(C)(C)O)c23)c1]

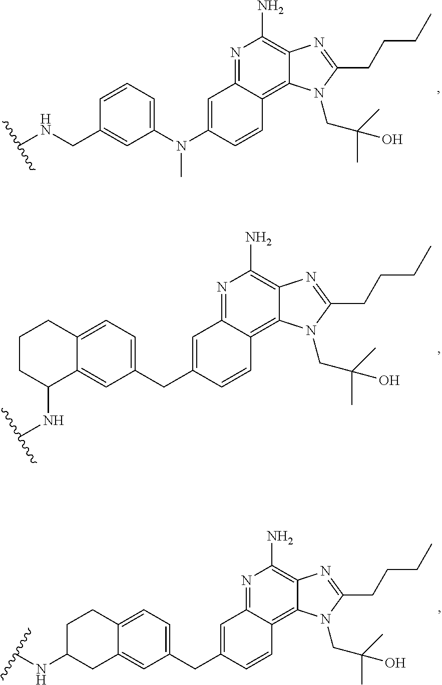 [SMILES: *NCc1cccc(N(C)c2ccc3c(c2)nc(N)c2nc(CCCC)n(CC(C)(C)O)c23)c1 ; *NC1CCCc2ccc(Cc3ccc4c(c3)nc(N)c3nc(CCCC)n(CC(C)(C)O)c34)cc21 ; *NC1CCc2ccc(Cc3ccc4c(c3)nc(N)c3nc(CCCC)n(CC(C)(C)O)c34)cc2C1]

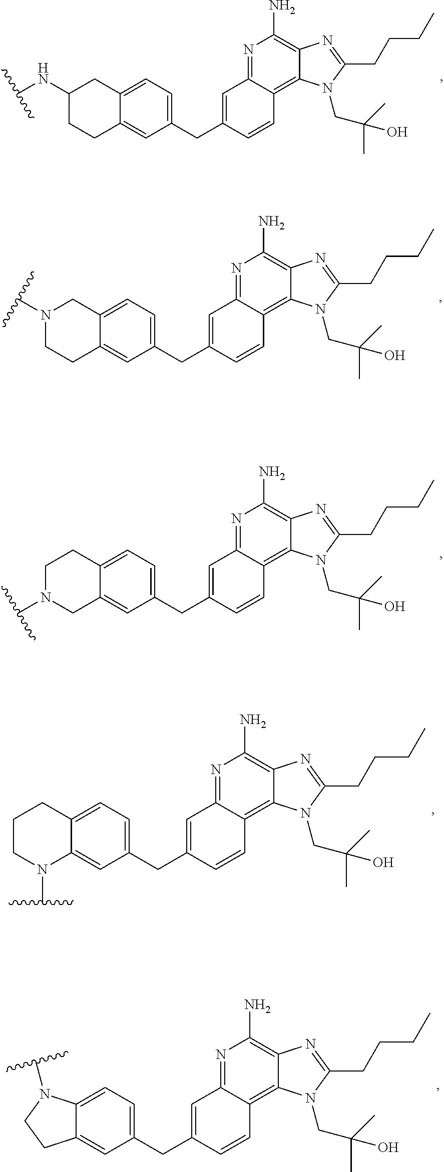 [SMILES: *NC1CCc2cc(Cc3ccc4c(c3)nc(N)c3nc(CCCC)n(CC(C)(C)O)c34)ccc2C1 ; *N1CCc2cc(Cc3ccc4c(c3)nc(N)c3nc(CCCC)n(CC(C)(C)O)c34)ccc2C1 ; *N1CCc2ccc(Cc3ccc4c(c3)nc(N)c3nc(CCCC)n(CC(C)(C)O)c34)cc2C1 ; *N1CCCc2ccc(Cc3ccc4c(c3)nc(N)c3nc(CCCC)n(CC(C)(C)O)c34)cc21 ; *N1CCc2cc(Cc3ccc4c(c3)nc(N)c3nc(CCCC)n(CC(C)(C)O)c34)ccc21]

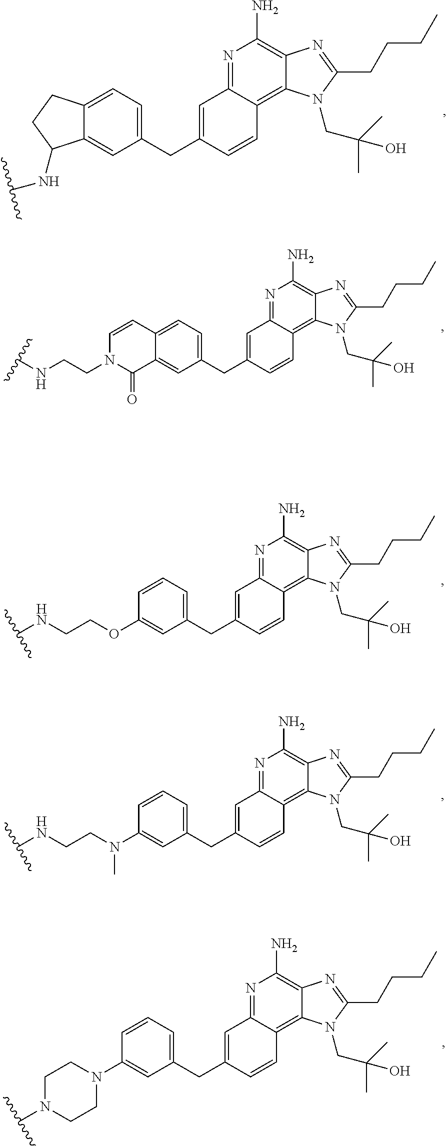 [SMILES: *NC1CCc2ccc(Cc3ccc4c(c3)nc(N)c3nc(CCCC)n(CC(C)(C)O)c34)cc21 ; *NCCn1ccc2ccc(Cc3ccc4c(c3)nc(N)c3nc(CCCC)n(CC(C)(C)O)c34)cc2c1=O ; *NCCOc1cccc(Cc2ccc3c(c2)nc(N)c2nc(CCCC)n(CC(C)(C)O)c23)c1 ; *NCCN(C)c1cccc(Cc2ccc3c(c2)nc(N)c2nc(CCCC)n(CC(C)(C)O)c23)c1 ; *N1CCN(c2cccc(Cc3ccc4c(c3)nc(N)c3nc(CCCC)n(CC(C)(C)O)c34)c2)CC1]

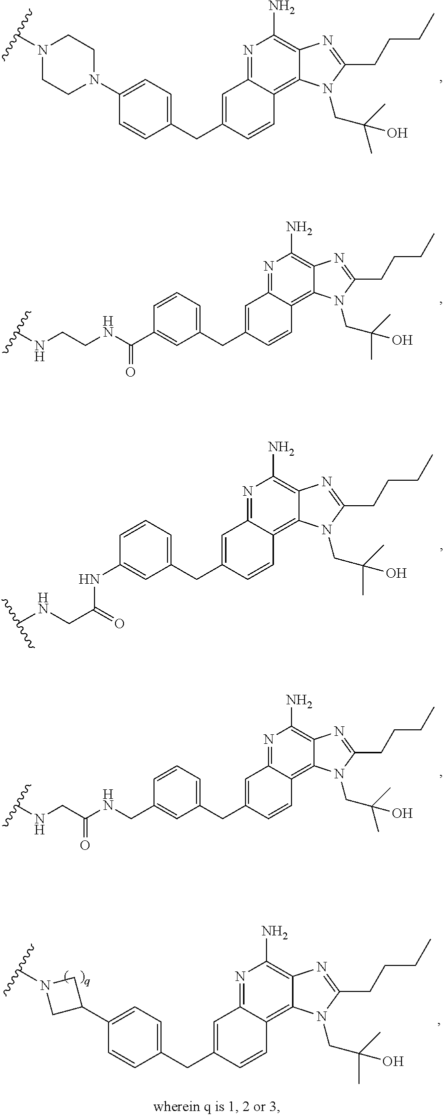

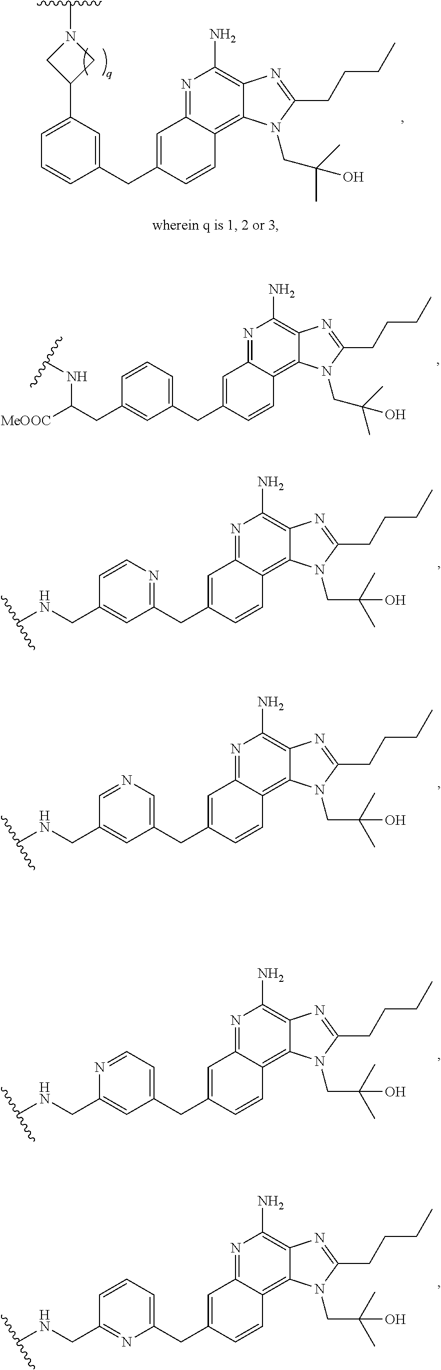

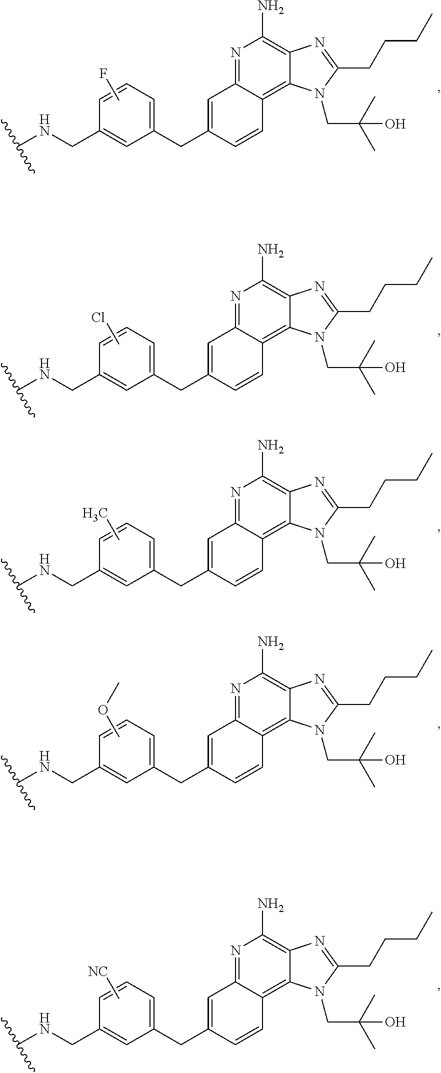

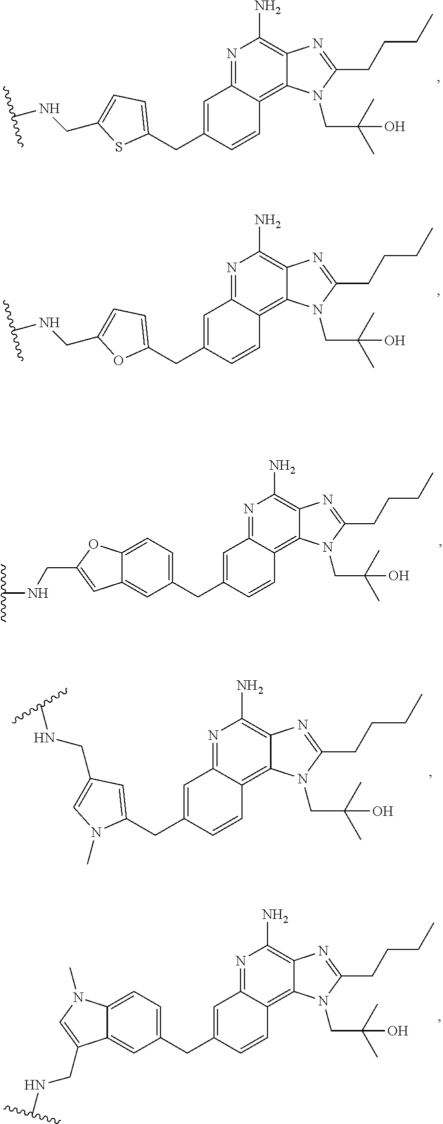 [SMILES: *NCc1ccc(Cc2ccc3c(c2)nc(N)c2nc(CCCC)n(CC(C)(C)O)c23)s1 ; *NCc1ccc(Cc2ccc3c(c2)nc(N)c2nc(CCCC)n(CC(C)(C)O)c23)o1 ; *NCc1cc2cc(Cc3ccc4c(c3)nc(N)c3nc(CCCC)n(CC(C)(C)O)c34)ccc2o1 ; *NCc1cc(Cc2ccc3c(c2)nc(N)c2nc(CCCC)n(CC(C)(C)O)c23)n(C)c1 ; *NCc1cn(C)c2ccc(Cc3ccc4c(c3)nc(N)c3nc(CCCC)n(CC(C)(C)O)c34)cc12]

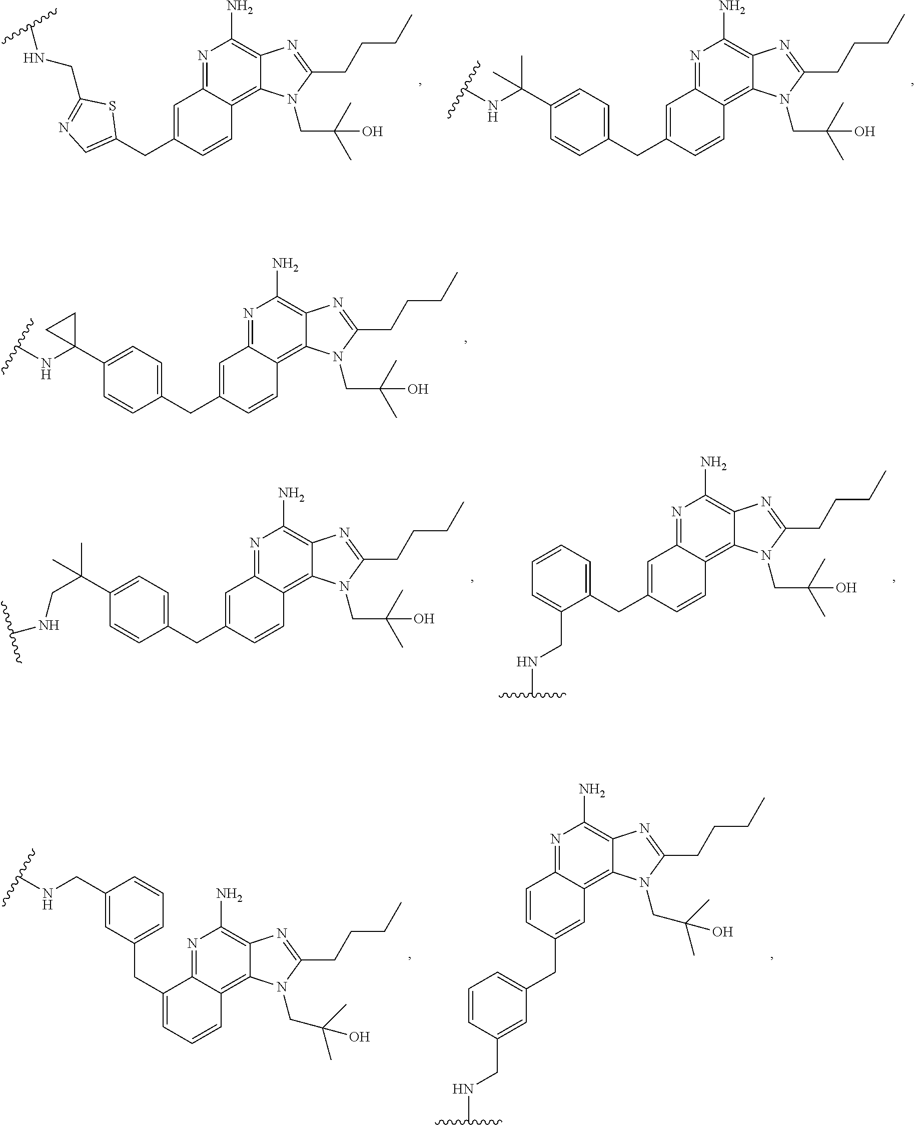

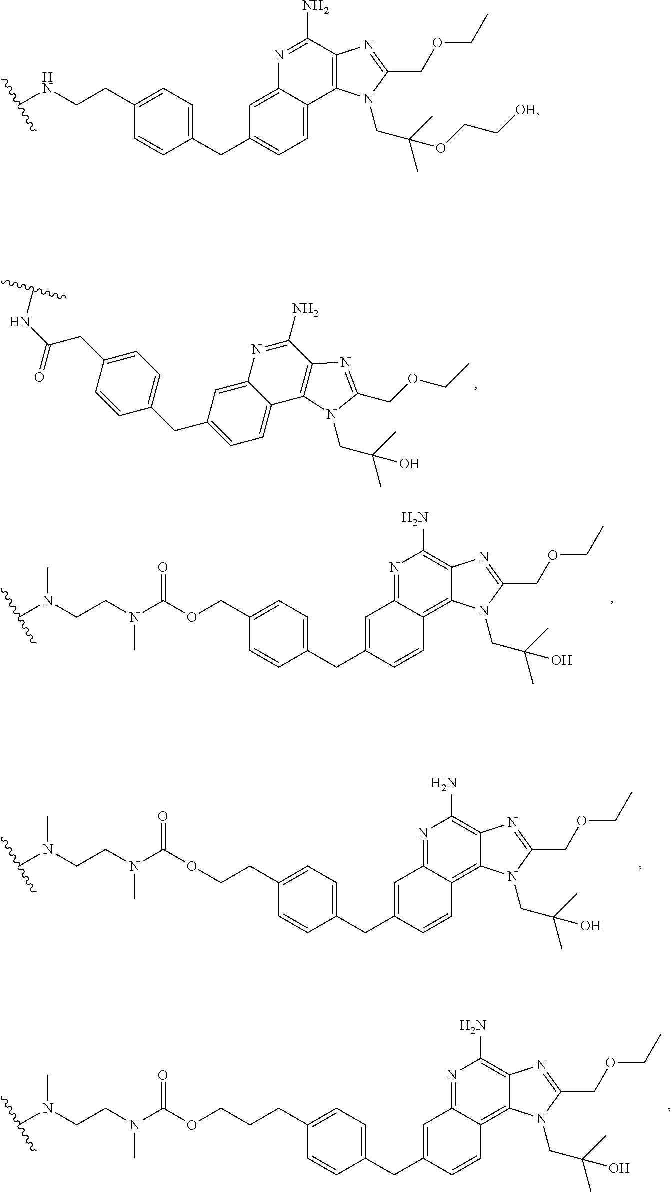 [SMILES: *NCCc1ccc(Cc2ccc3c(c2)nc(N)c2nc(COCC)n(CC(C)(C)OCCO)c23)cc1 ; *NC(=O)Cc1ccc(Cc2ccc3c(c2)nc(N)c2nc(COCC)n(CC(C)(C)O)c23)cc1 ; *N(C)CCN(C)C(=O)OCc1ccc(Cc2ccc3c(c2)nc(N)c2nc(COCC)n(CC(C)(C)O)c23)cc1 ; *N(C)CCN(C)C(=O)OCCc1ccc(Cc2ccc3c(c2)nc(N)c2nc(COCC)n(CC(C)(C)O)c23)cc1 ; *N(C)CCN(C)C(=O)OCCCc1ccc(Cc2ccc3c(c2)nc(N)c2nc(COCC)n(CC(C)(C)O)c23)cc1]

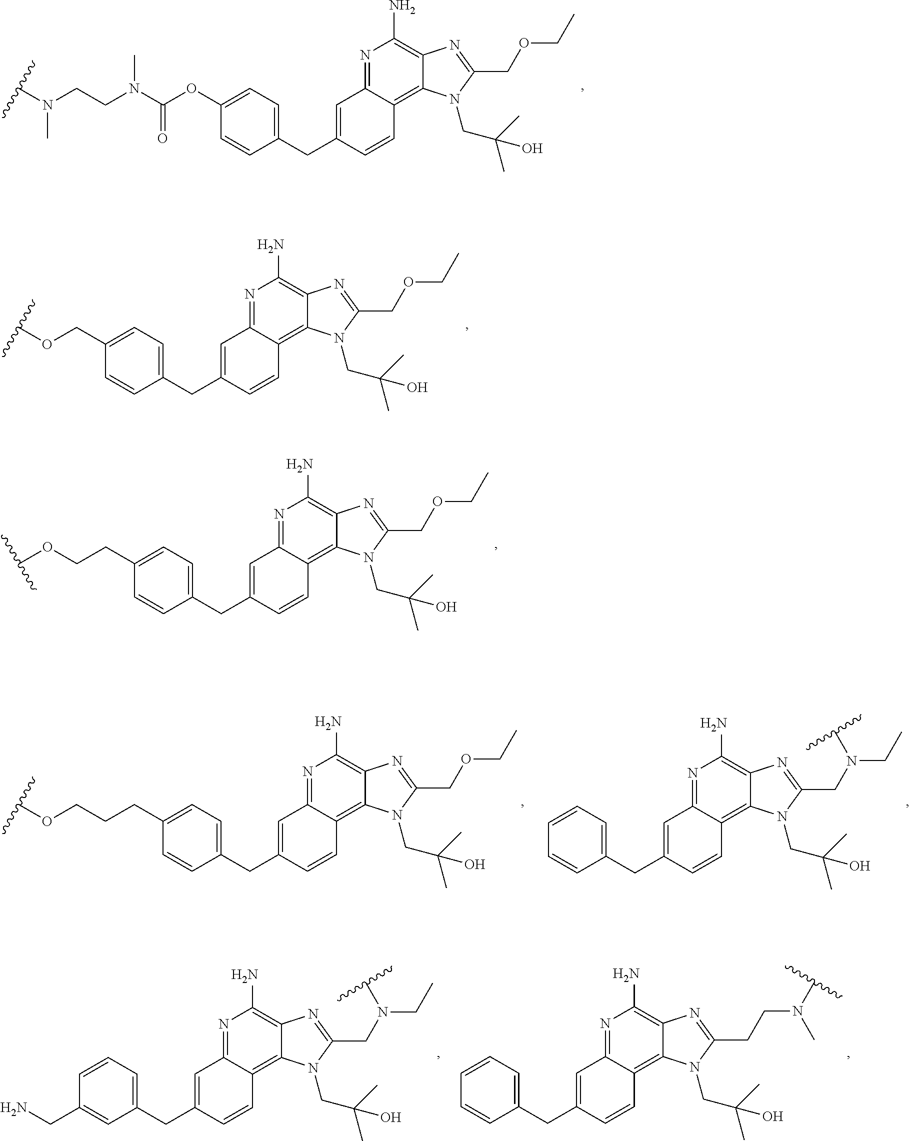

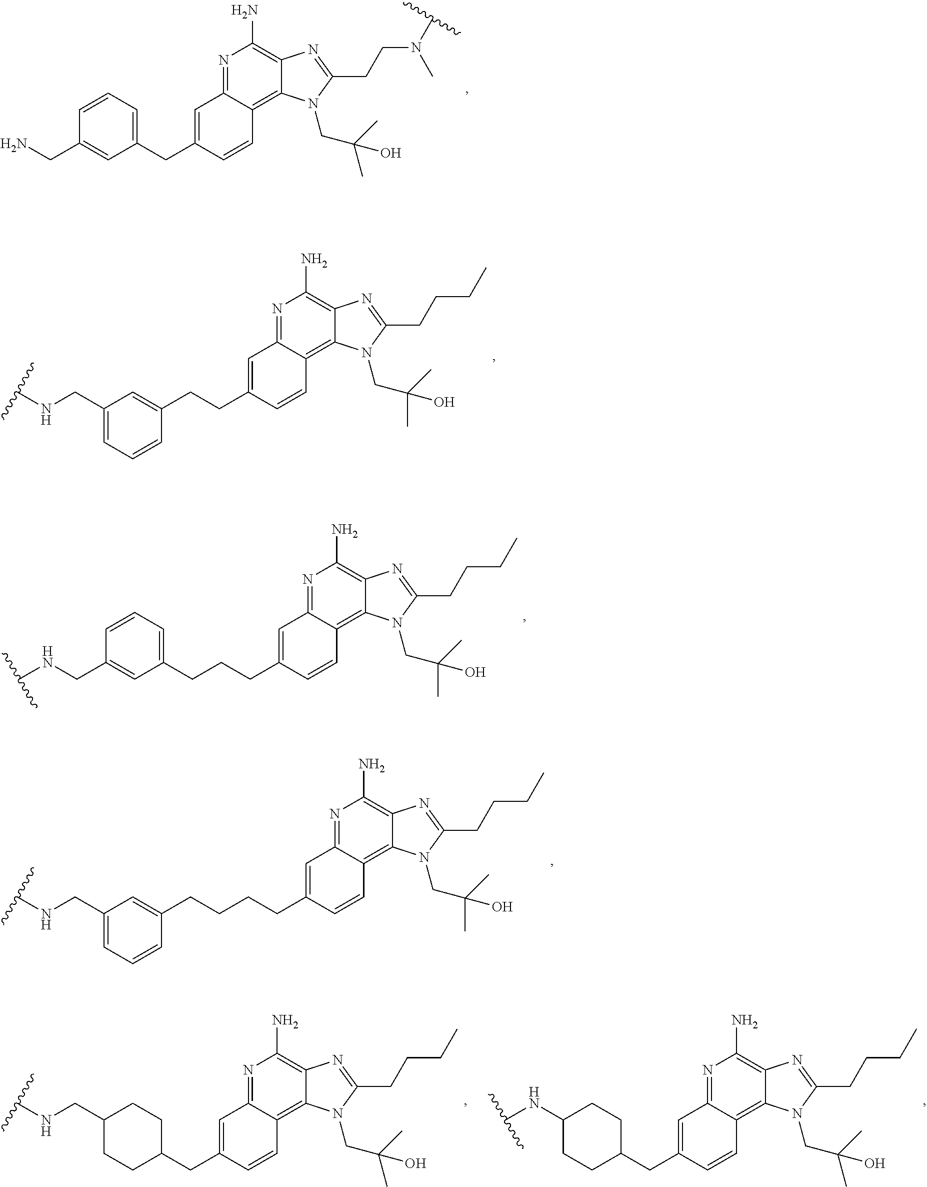 [SMILES: *N(C)CCc1nc2c(N)nc3cc(Cc4cccc(CN)c4)ccc3c2n1CC(C)(C)O ; *NCc1cccc(CCc2ccc3c(c2)nc(N)c2nc(CCCC)n(CC(C)(C)O)c23)c1 ; *NCc1cccc(CCCc2ccc3c(c2)nc(N)c2nc(CCCC)n(CC(C)(C)O)c23)c1 ; *NCc1cccc(CCCCc2ccc3c(c2)nc(N)c2nc(CCCC)n(CC(C)(C)O)c23)c1 ; *NCC1CCC(Cc2ccc3c(c2)nc(N)c2nc(CCCC)n(CC(C)(C)O)c23)CC1 ; *NC1CCC(Cc2ccc3c(c2)nc(N)c2nc(CCCC)n(CC(C)(C)O)c23)CC1]

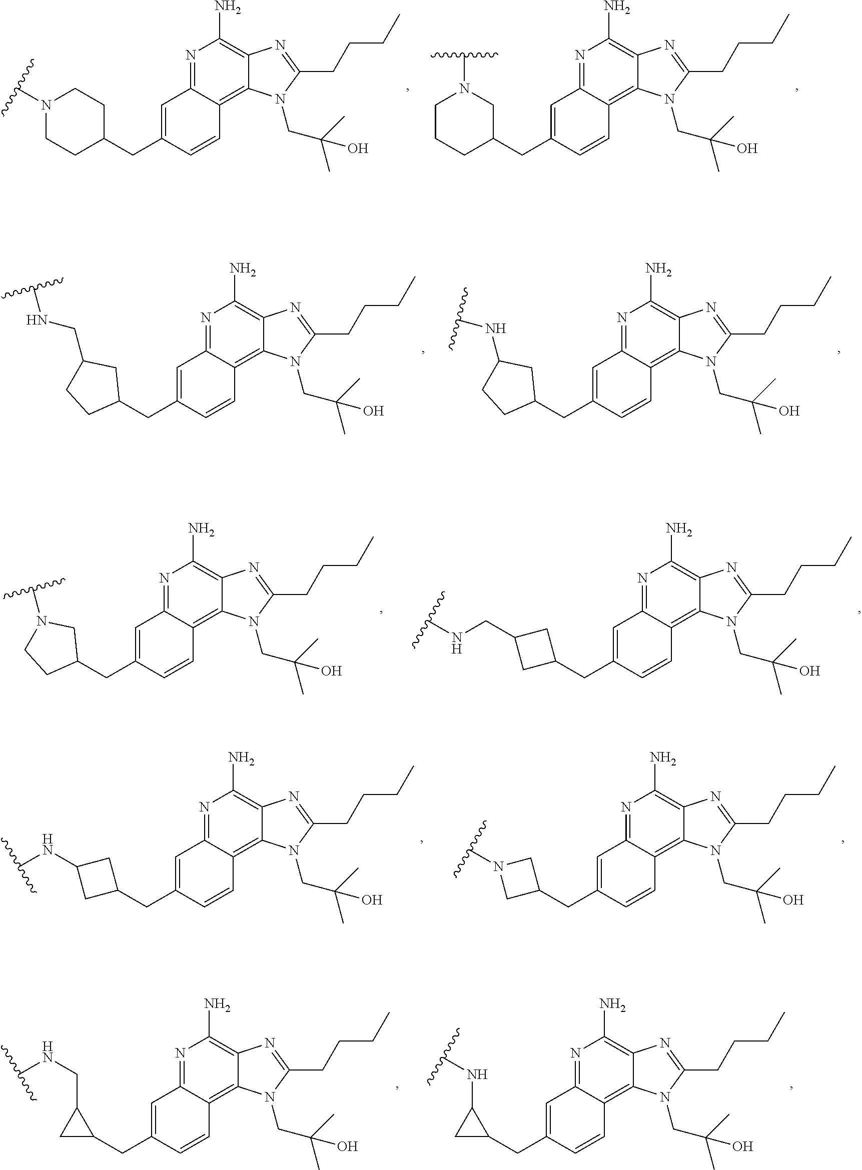

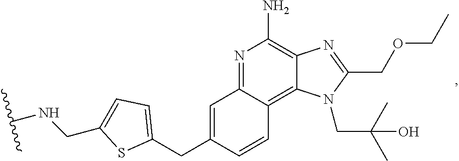 [SMILES: *NCc1ccc(Cc2ccc3c(c2)nc(N)c2nc(COCC)n(CC(C)(C)O)c23)s1]

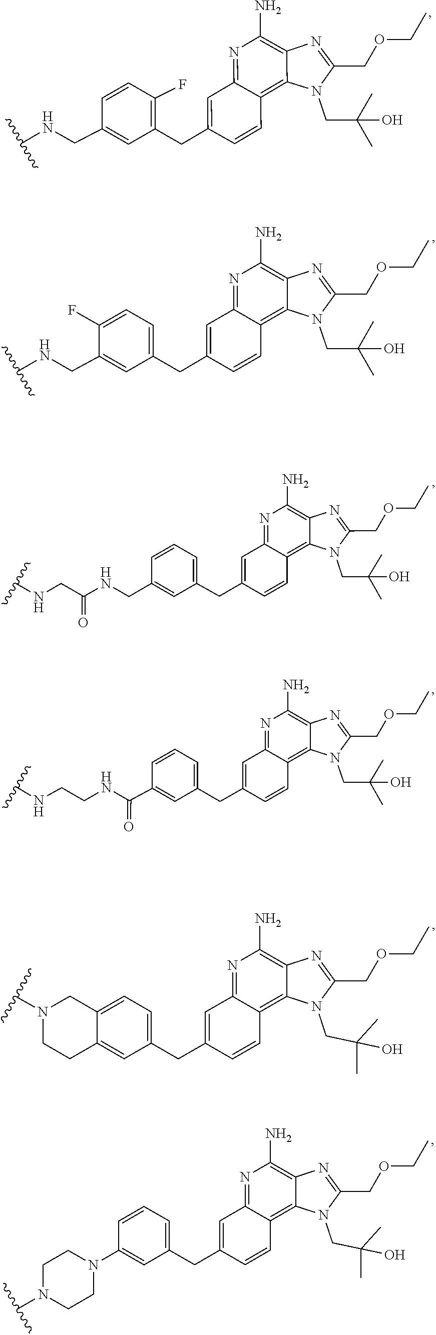 [SMILES: *NCc1ccc(F)c(Cc2ccc3c(c2)nc(N)c2nc(COCC)n(CC(C)(C)O)c23)c1 ; *NCc1cc(Cc2ccc3c(c2)nc(N)c2nc(COCC)n(CC(C)(C)O)c23)ccc1F ; *NCC(=O)NCc1cccc(Cc2ccc3c(c2)nc(N)c2nc(COCC)n(CC(C)(C)O)c23)c1 ; *NCCNC(=O)c1cccc(Cc2ccc3c(c2)nc(N)c2nc(COCC)n(CC(C)(C)O)c23)c1 ; *N1CCc2cc(Cc3ccc4c(c3)nc(N)c3nc(COCC)n(CC(C)(C)O)c34)ccc2C1 ; *N1CCN(c2cccc(Cc3ccc4c(c3)nc(N)c3nc(COCC)n(CC(C)(C)O)c34)c2)CC1]

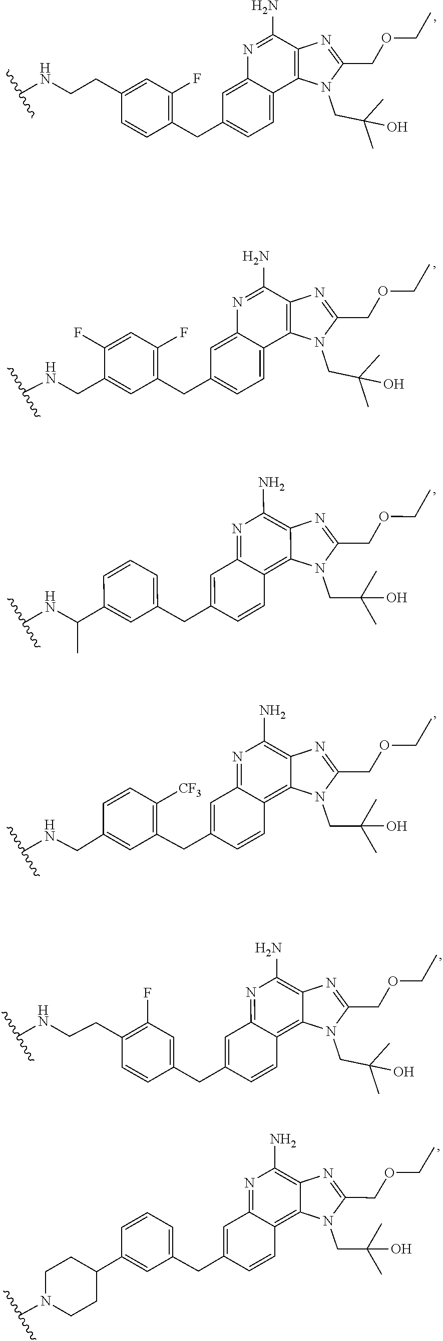 [SMILES: *NCCc1ccc(Cc2ccc3c(c2)nc(N)c2nc(COCC)n(CC(C)(C)O)c23)c(F)c1 ; *NCc1cc(Cc2ccc3c(c2)nc(N)c2nc(COCC)n(CC(C)(C)O)c23)c(F)cc1F ; *NC(C)c1cccc(Cc2ccc3c(c2)nc(N)c2nc(COCC)n(CC(C)(C)O)c23)c1 ; *NCc1ccc(C(F)(F)F)c(Cc2ccc3c(c2)nc(N)c2nc(COCC)n(CC(C)(C)O)c23)c1 ; *NCCc1ccc(Cc2ccc3c(c2)nc(N)c2nc(COCC)n(CC(C)(C)O)c23)cc1F ; *N1CCC(c2cccc(Cc3ccc4c(c3)nc(N)c3nc(COCC)n(CC(C)(C)O)c34)c2)CC1]

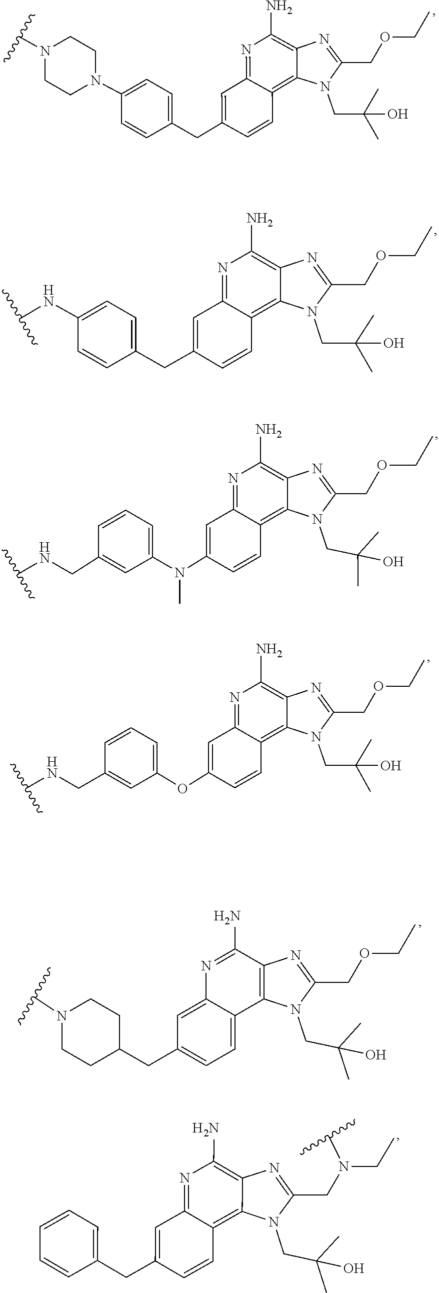 [SMILES: *N1CCN(c2ccc(Cc3ccc4c(c3)nc(N)c3nc(COCC)n(CC(C)(C)O)c34)cc2)CC1 ; *Nc1ccc(Cc2ccc3c(c2)nc(N)c2nc(COCC)n(CC(C)(C)O)c23)cc1 ; *NCc1cccc(N(C)c2ccc3c(c2)nc(N)c2nc(COCC)n(CC(C)(C)O)c23)c1 ; *NCc1cccc(Oc2ccc3c(c2)nc(N)c2nc(COCC)n(CC(C)(C)O)c23)c1 ; *N1CCC(Cc2ccc3c(c2)nc(N)c2nc(COCC)n(CC(C)(C)O)c23)CC1 ; *N(CC)Cc1nc2c(N)nc3cc(Cc4ccccc4)ccc3c2n1CC(C)(C)O]

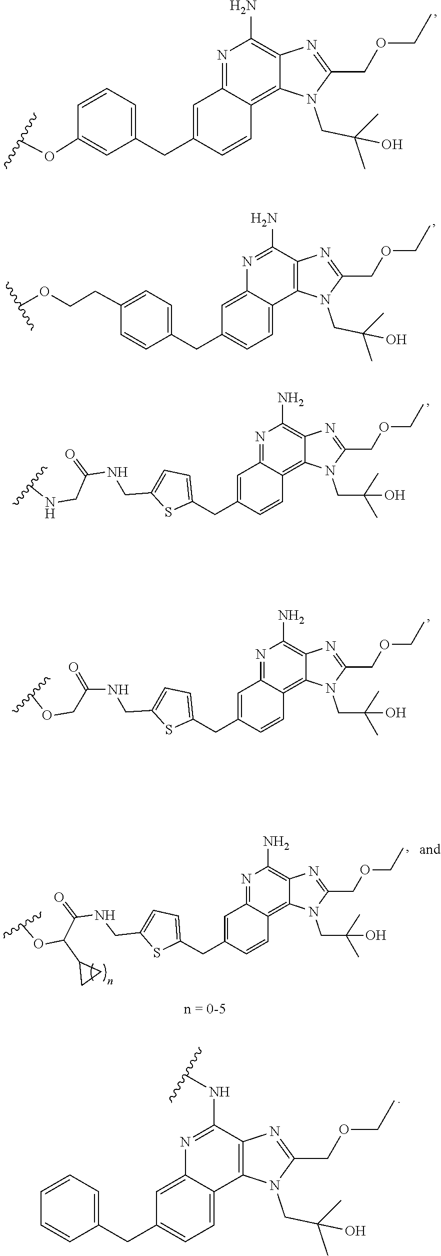

212. The conjugate compound of claim 202, wherein L has Formula (III): 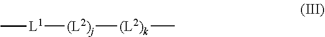
wherein
L¹ is a stretcher unit covalently attached to the targeting moiety;
L² is an optional peptide unit of two to twelve amino acid residues,
L³ is an optional spacer unit covalently attached to the payload unit, and
j and k are independently selected from 0 and 1; and/or
L¹ is selected from the group consisting of: 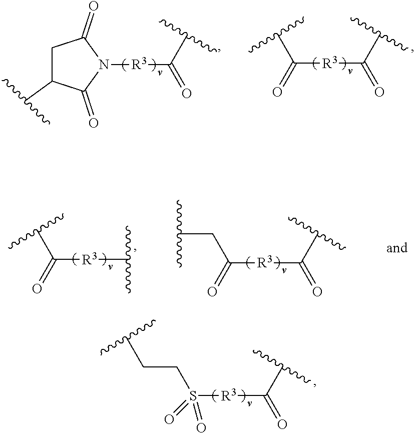
wherein each R³ is independently selected from the group consisting of alkyl, heteroalkyl, cycloalkyl, heterocyclyl, aryl, heteroaryl, alkylaryl, arylalkyl, alkylcycloalkyl, cycloalkylalkyl, alkylheterocyclyl, heterocyclylalkyl, -alkyl-C(O)N(R^{a})-alkyl-N(R^{a}), —N(R^{a})-alkyl-, and —(CH₂CH₂O)ᵣ—CH₂—, wherein R^{a} is H or alkyl, and r is an integer ranging from 1 to 10, v is an integer ranging from 0 to 5, optionally each R³ is independently selected from the group consisting of C₁₋₁₀ alkyl, C₁₋₈ heteroalkyl, C₃₋₈ cycloalkyl, 3 to 8 membered heterocyclyl, aryl, heteroaryl, (C₁₋₁₀ alkyl)aryl, aryl(C₁₋₁₀ alkyl), (C₁₋₁₀ alkyl)(C₃₋₈ cycloalkyl), (C₃₋₈ cycloalkyl)(C₁₋₁₀ alkyl), (C₁₋₁₀ alkyl)(3 to 8 membered heterocyclyl), (3 to 8 membered heterocyclyl)(C₁₋₁₀ alkyl), —(C₂₋₆ alkyl)-C(O)N(R^{a})—(C₂₋₆ alkyl)-N(R^{a}), —N(R^{a})—(C₂₋₆ alkyl)-, and —(CH₂CH₂O)ᵣ—CH₂—, wherein R^{a} is H or C₁₋₆ alkyl.

213. The conjugate compound of claim 202 having the formula: 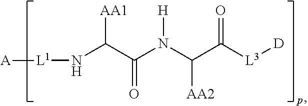
wherein AA1 and AA2 are independently selected from an amino acid side chain; p is an integer from 1 to 8, optionally the amino acid side chain is independently selected from H, —CH₃, —CH₂(C₆H₅), —CH₂CH₂CH₂CH₂NH₂, —CH₂CH₂CH₂NHC(NH)NH₂, —CHCH(CH₃)CH₃, and —CH₂CH₂CH₂NHC(O)NH₂.

214. The conjugate compound of claim 202 is selected from the group consisting of: 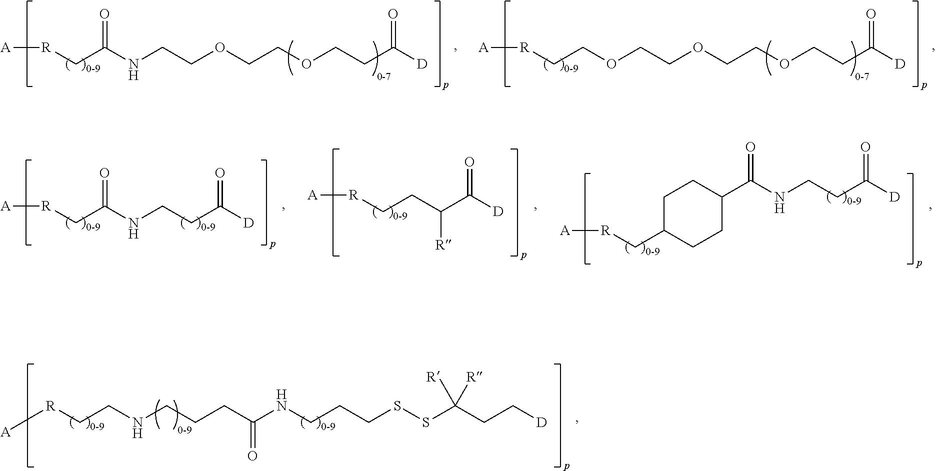 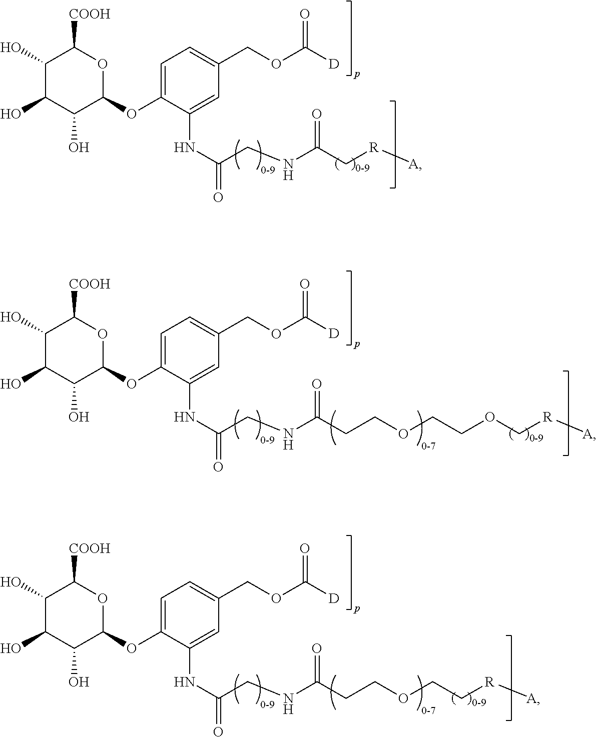 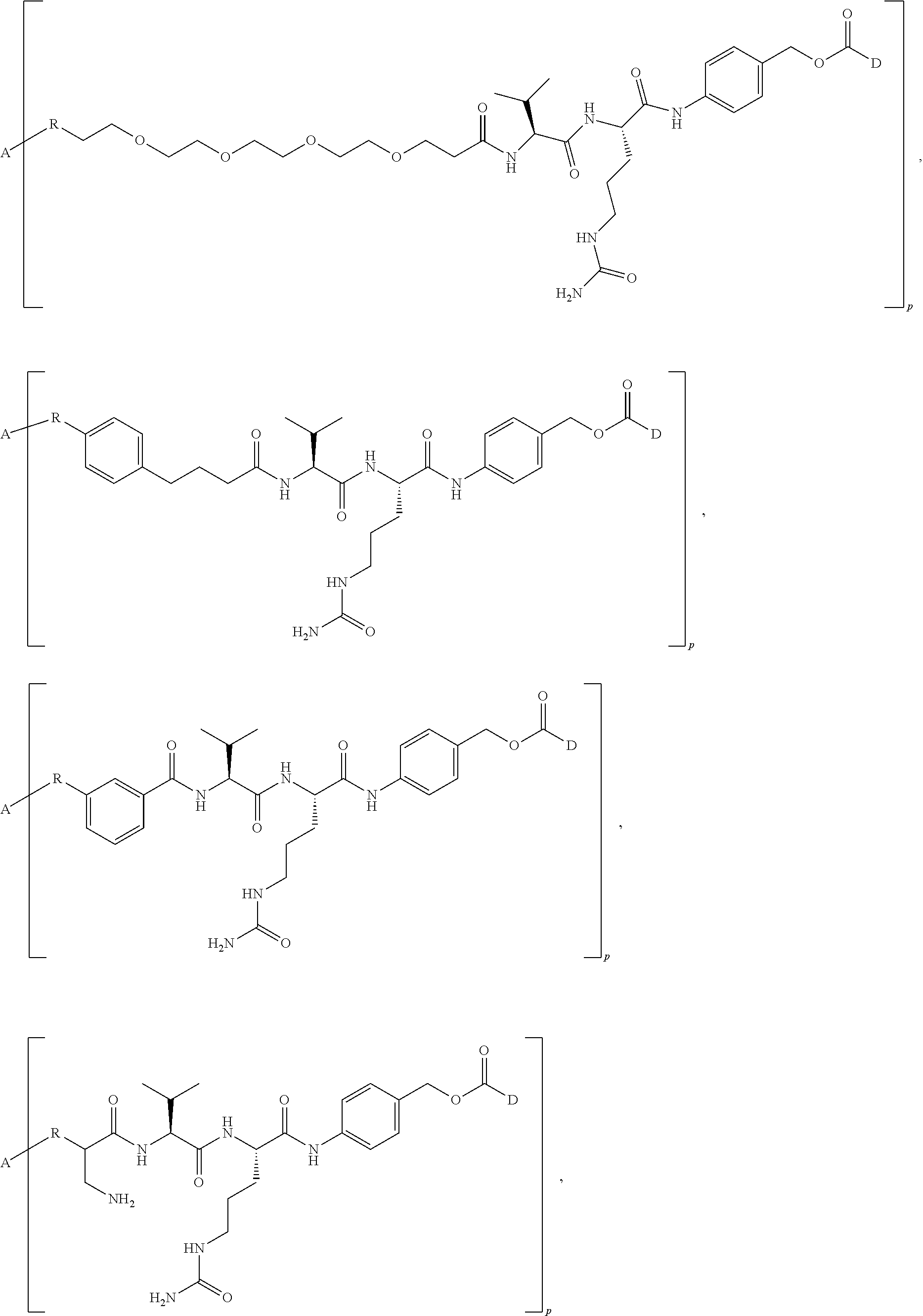 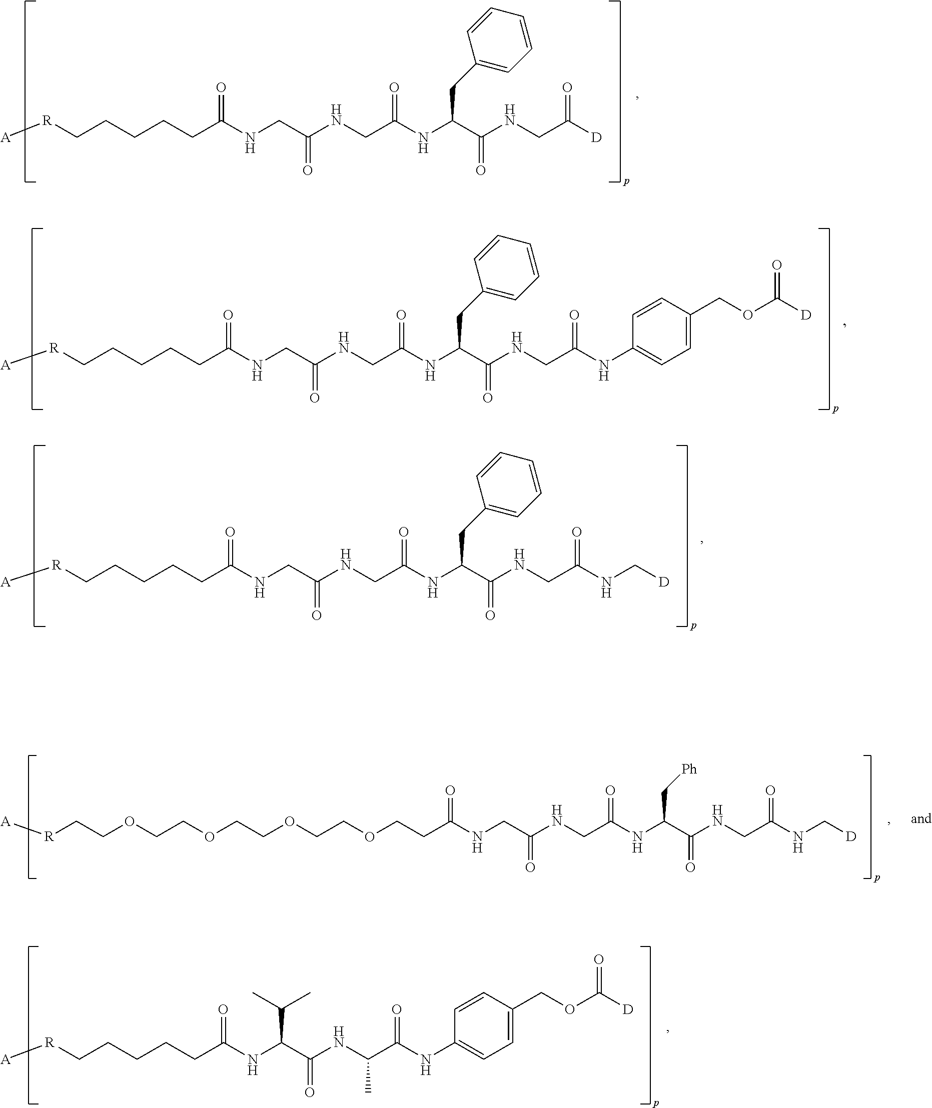
wherein R is selected from the group consisting of: 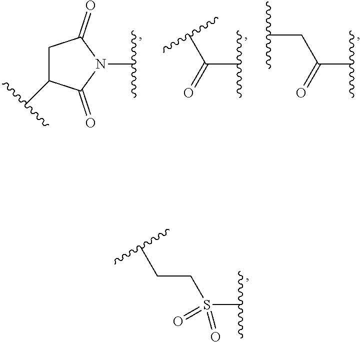
wherein each of R′ and R″ is independently a bond, hydrogen or methyl;

215. The conjugate compound of claim 202 is selected from the group consisting of: 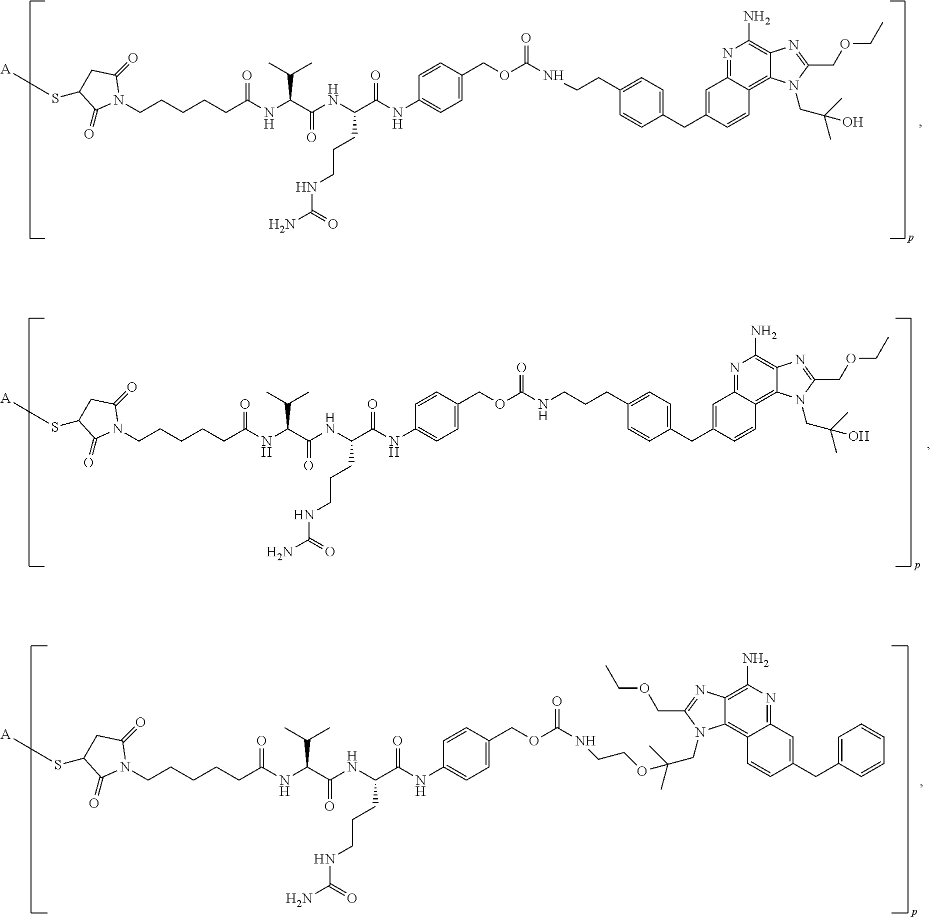 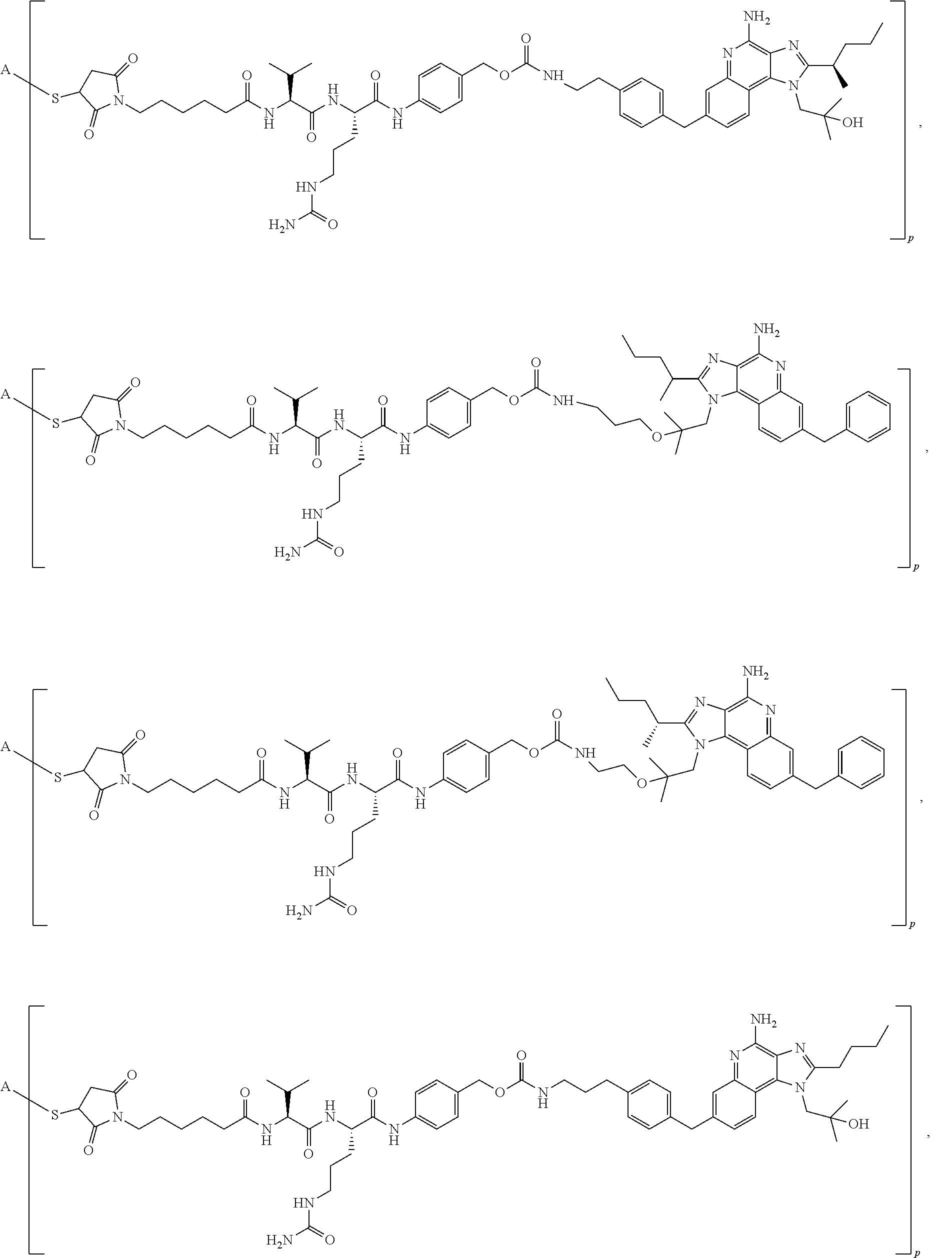 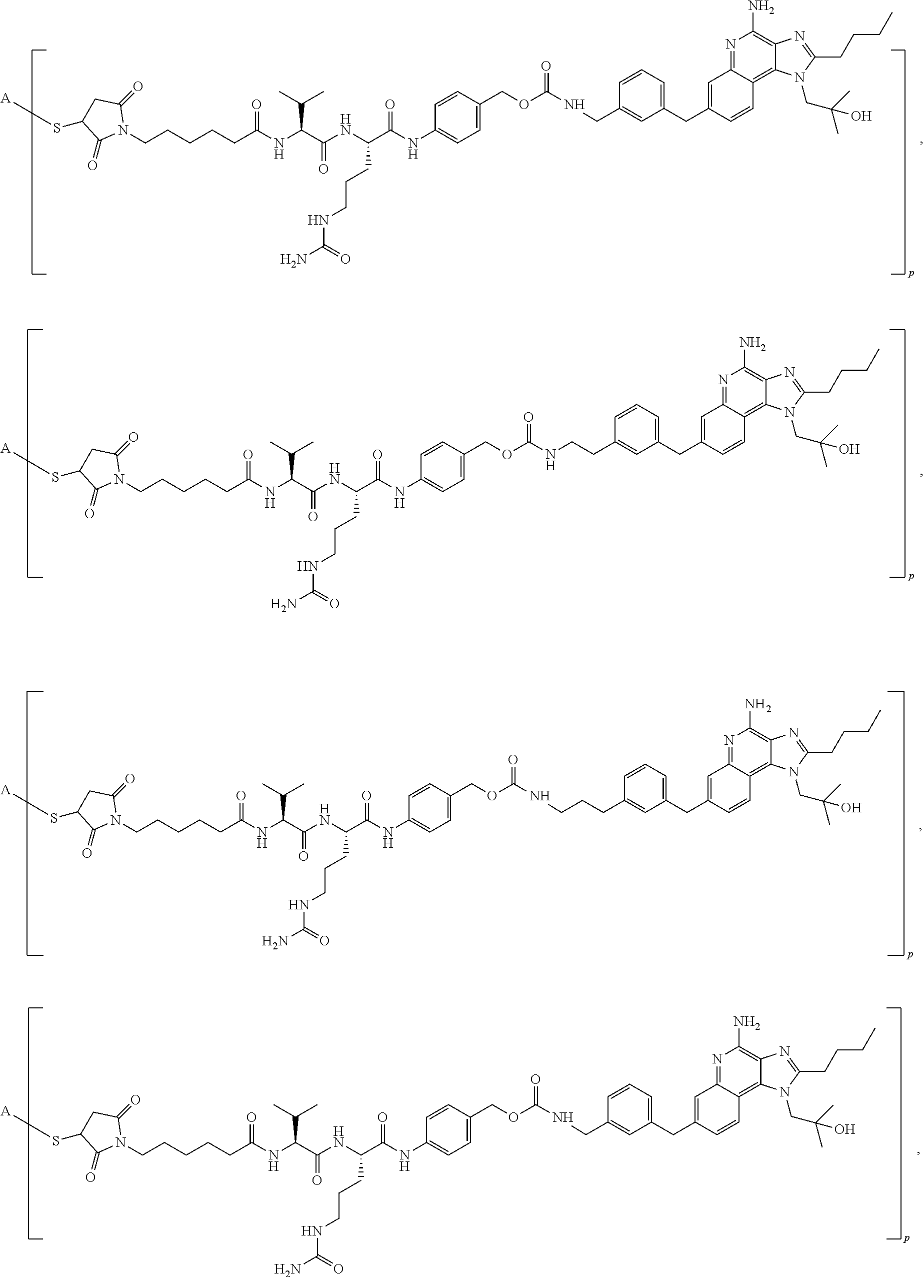 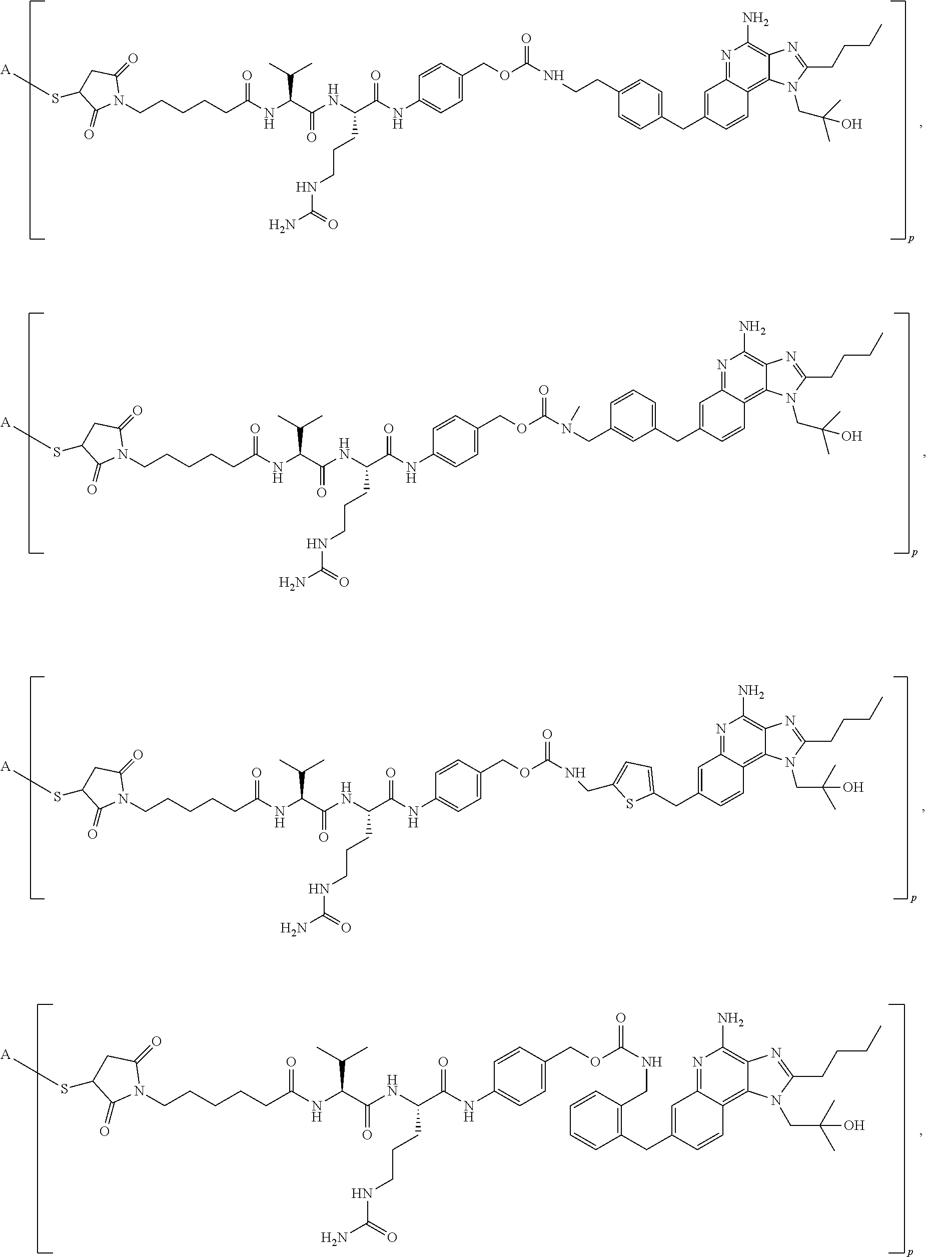 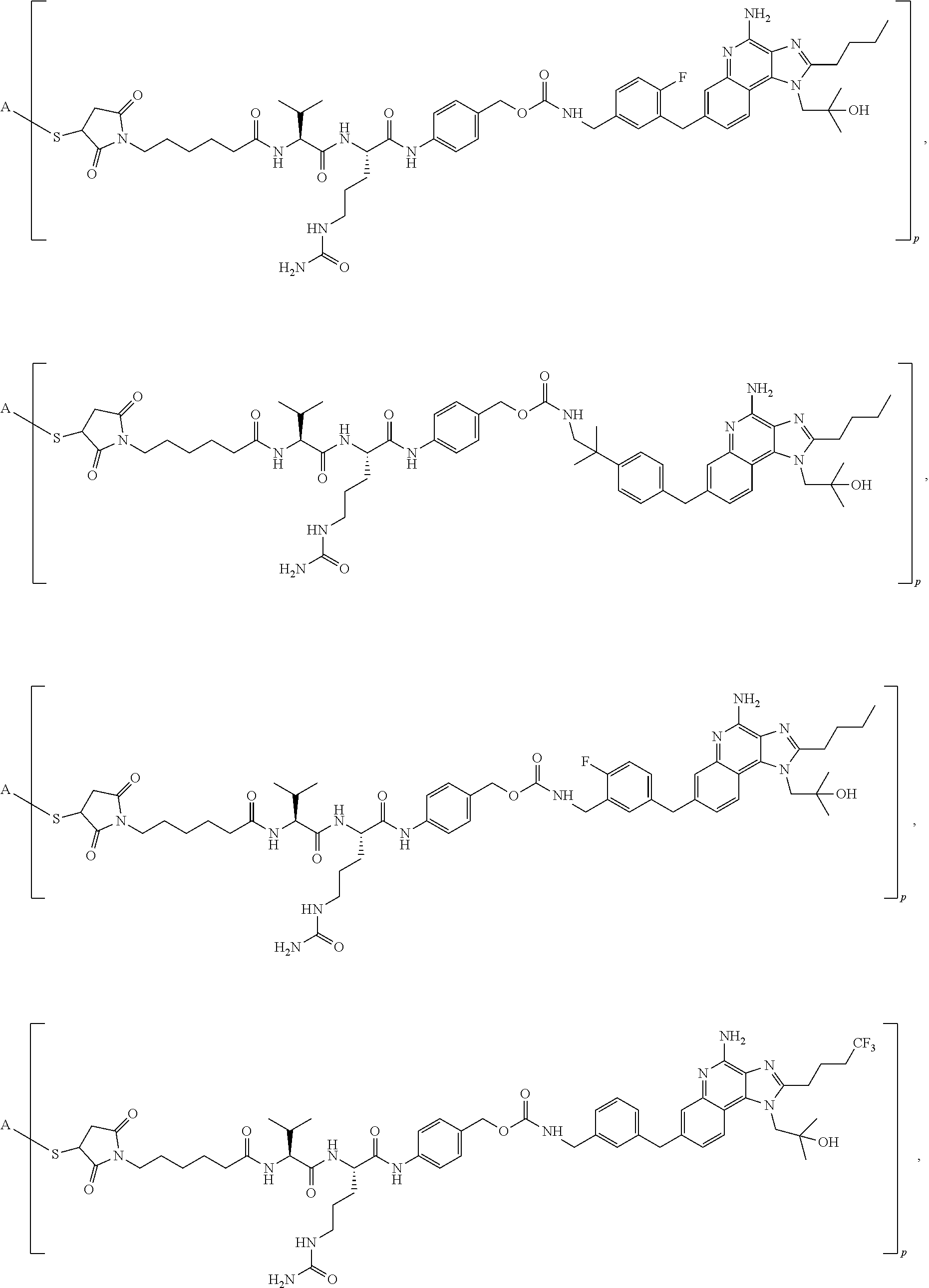 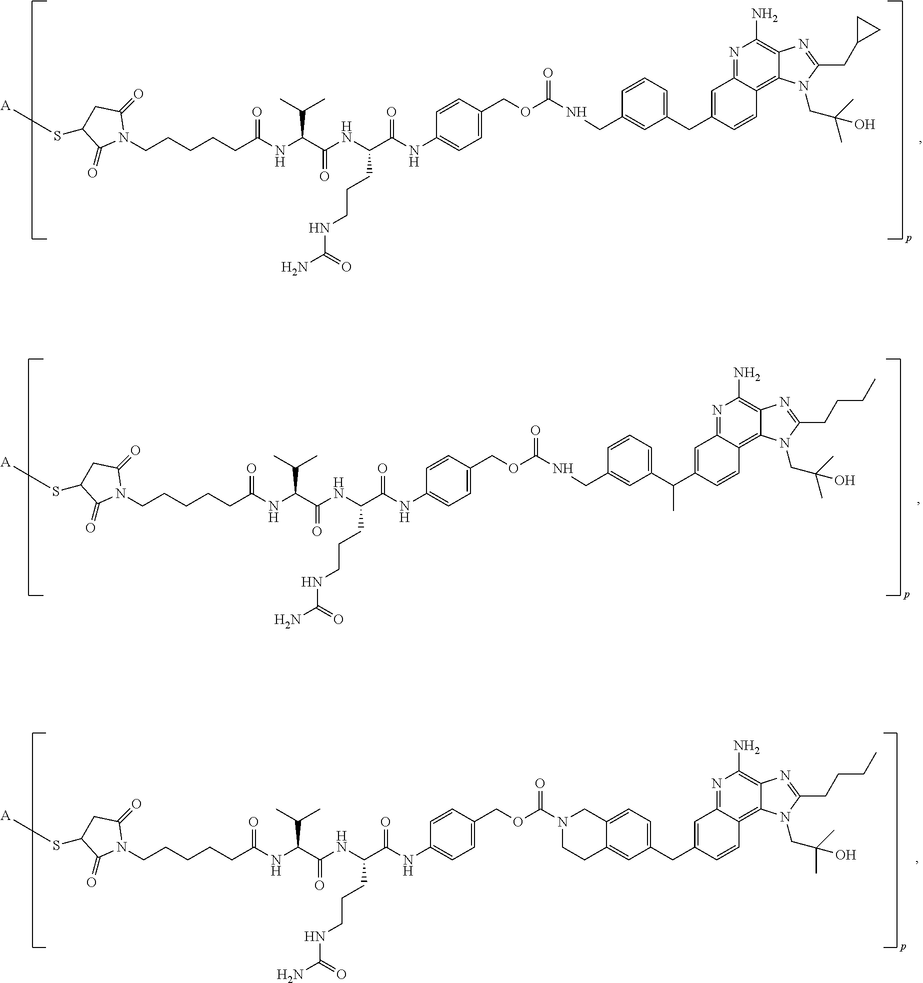 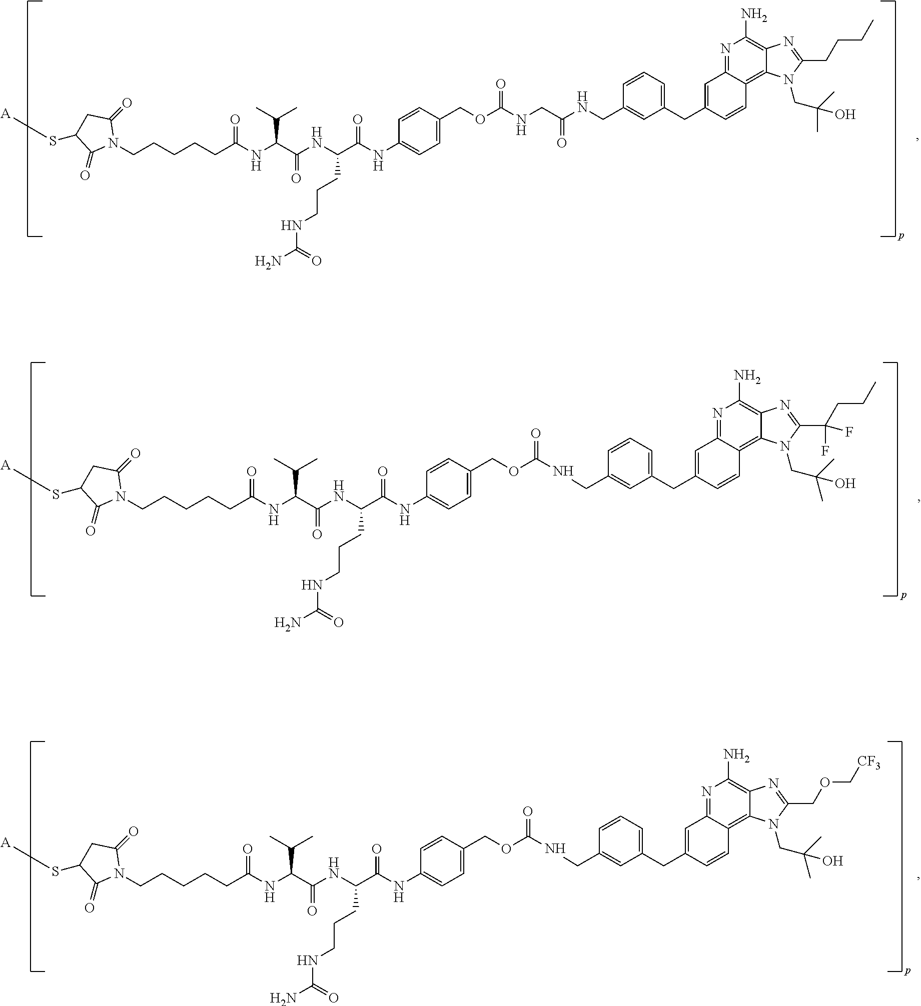 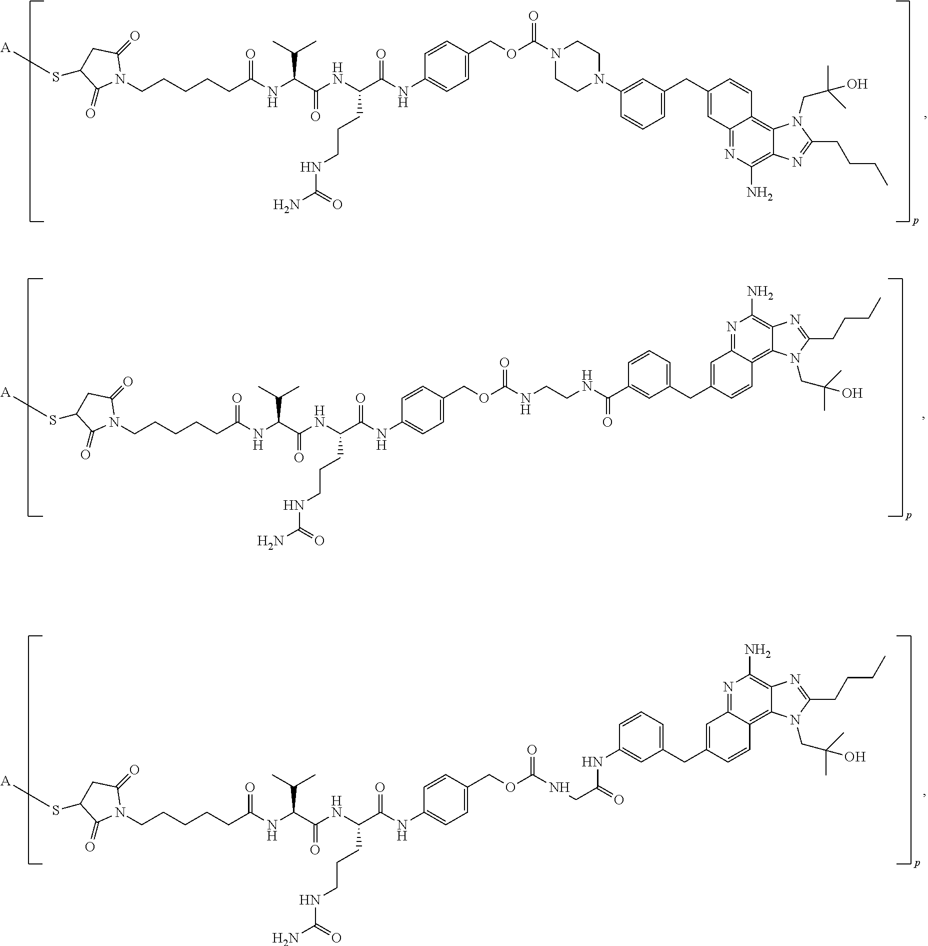 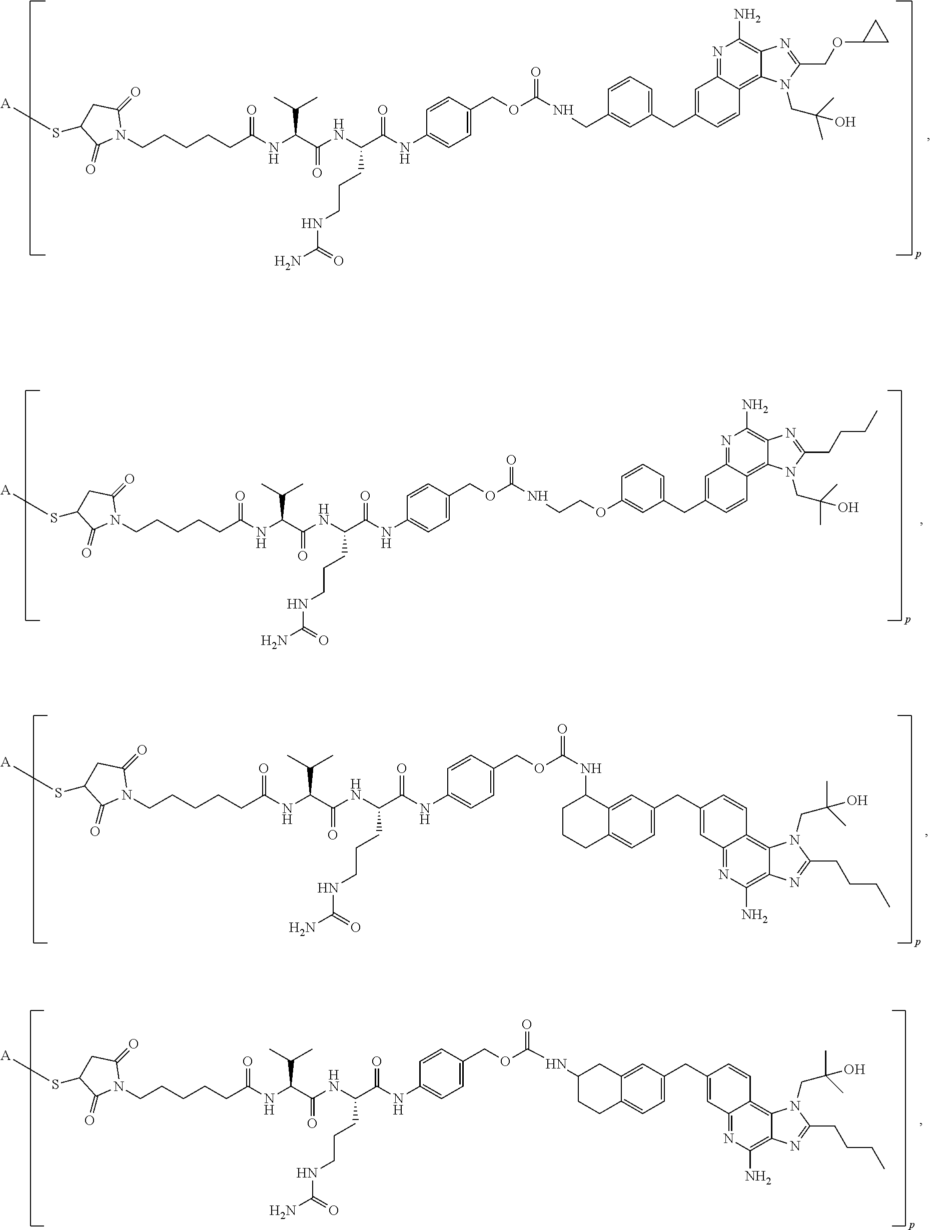 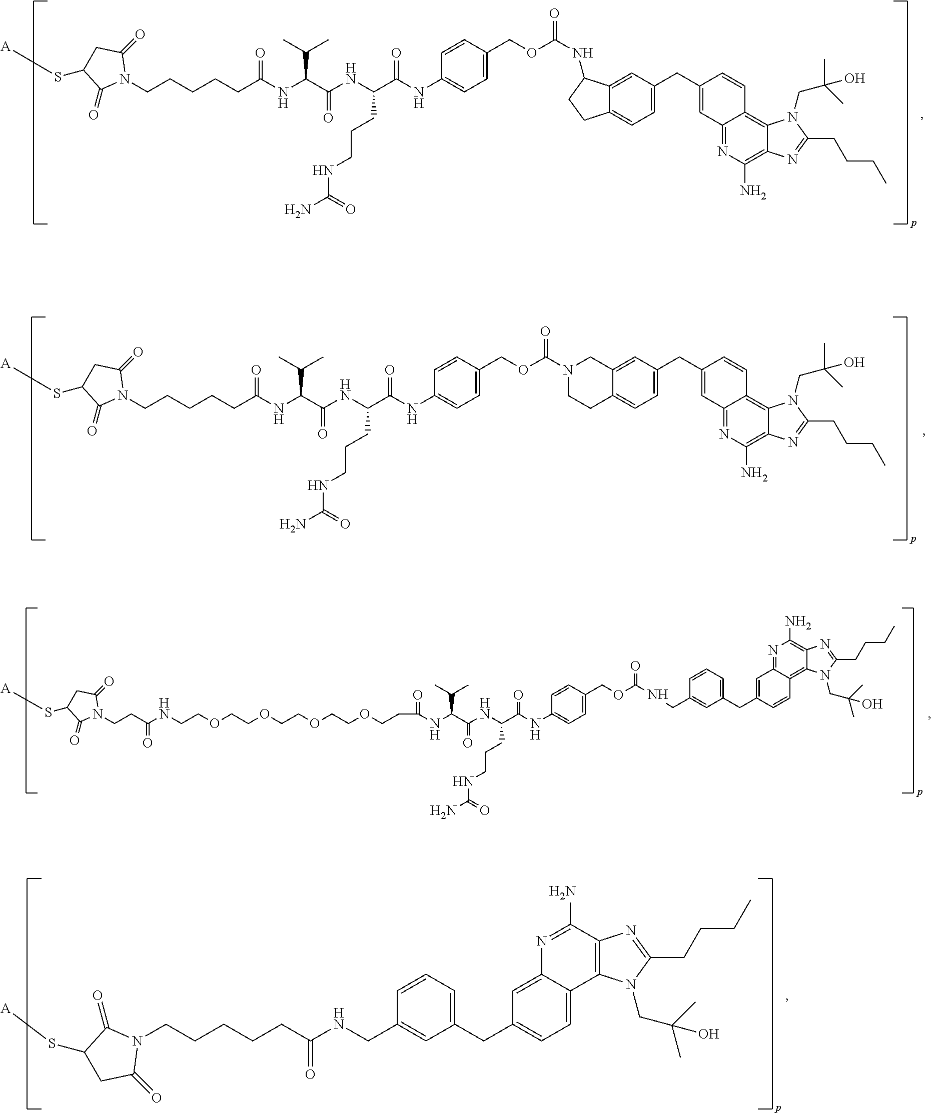 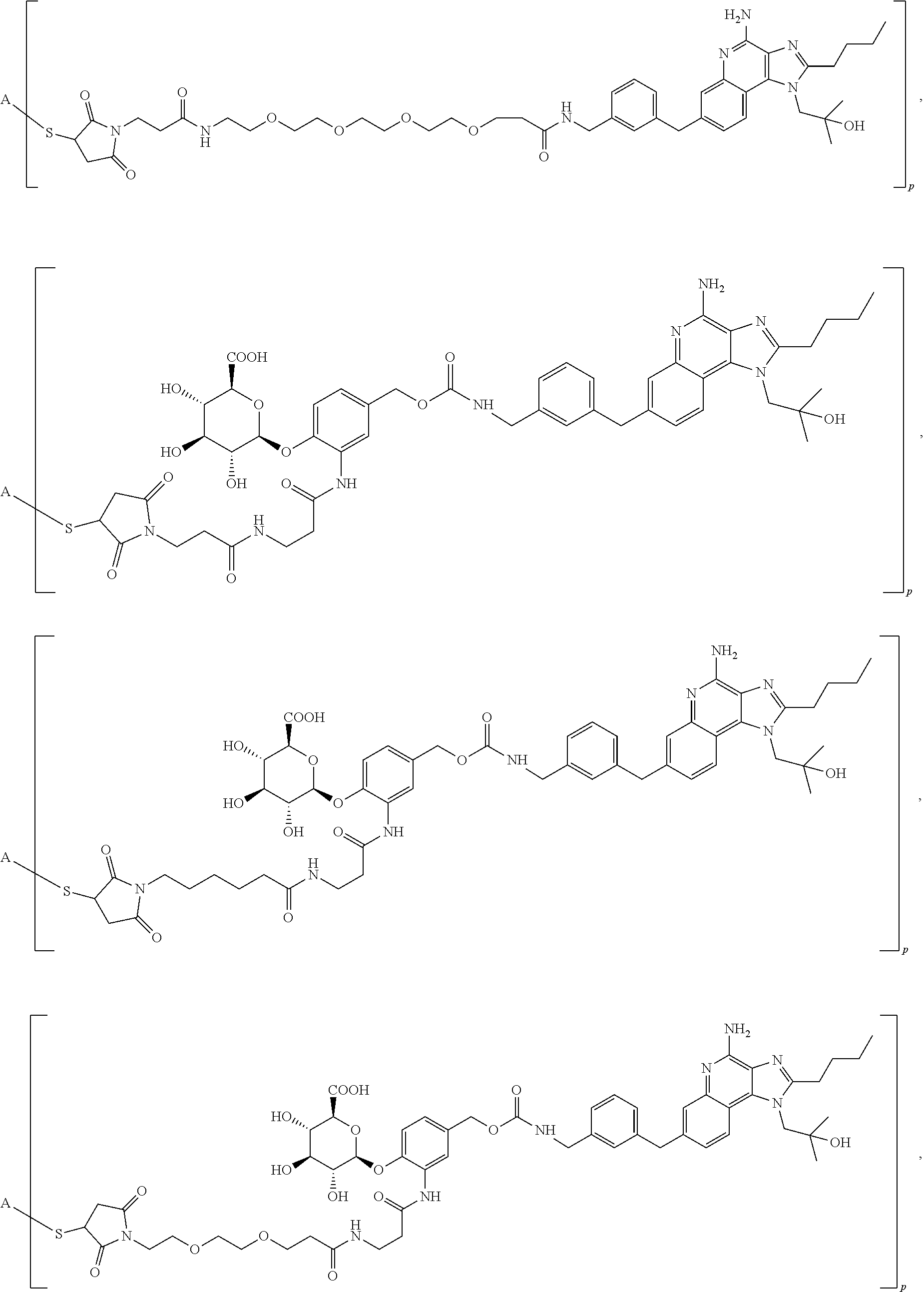 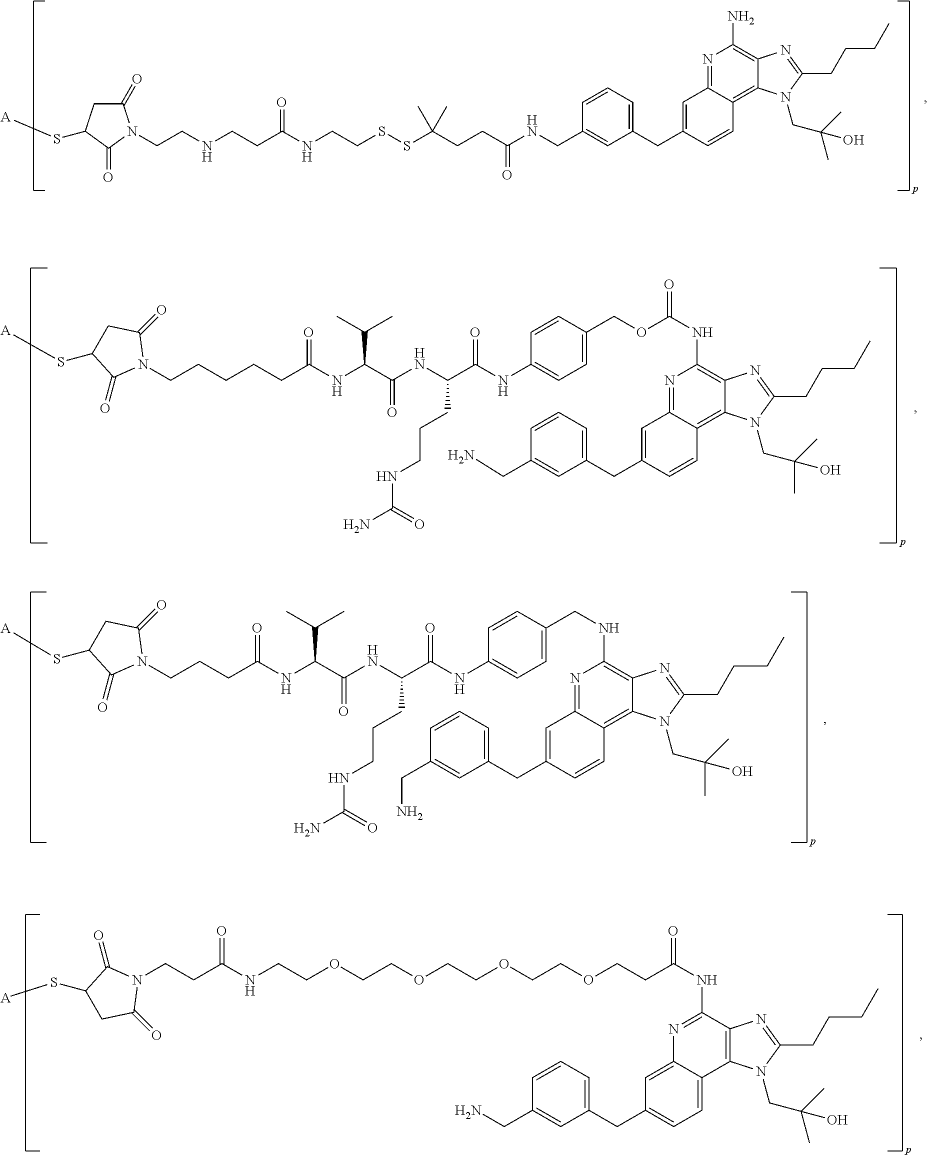 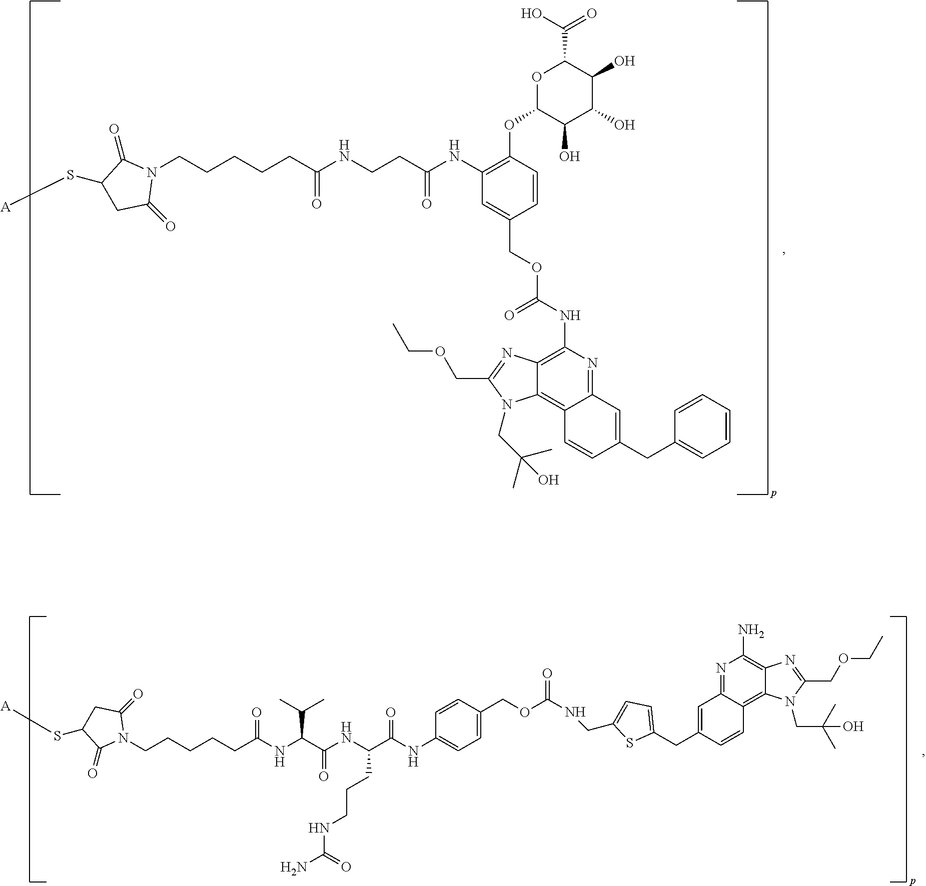 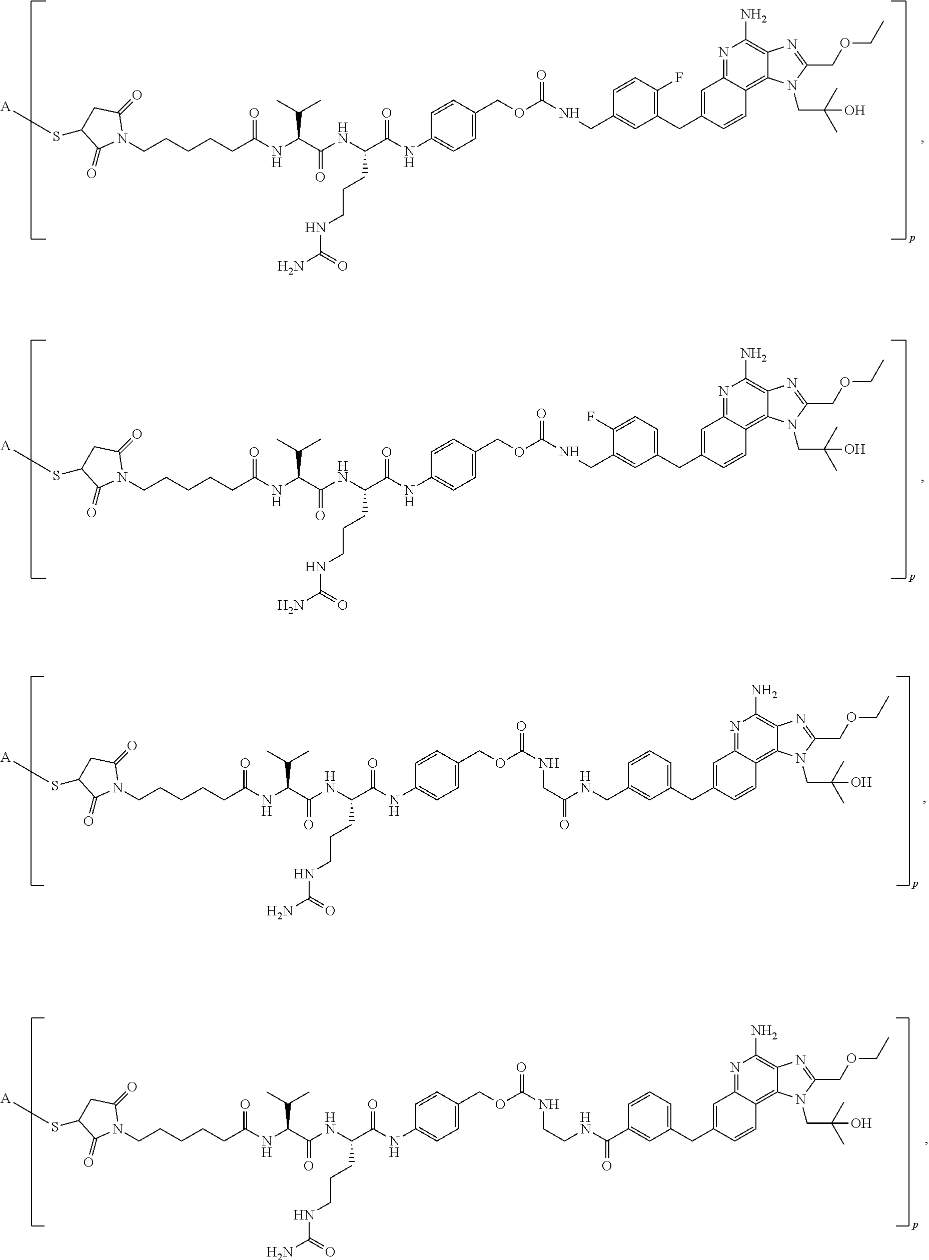 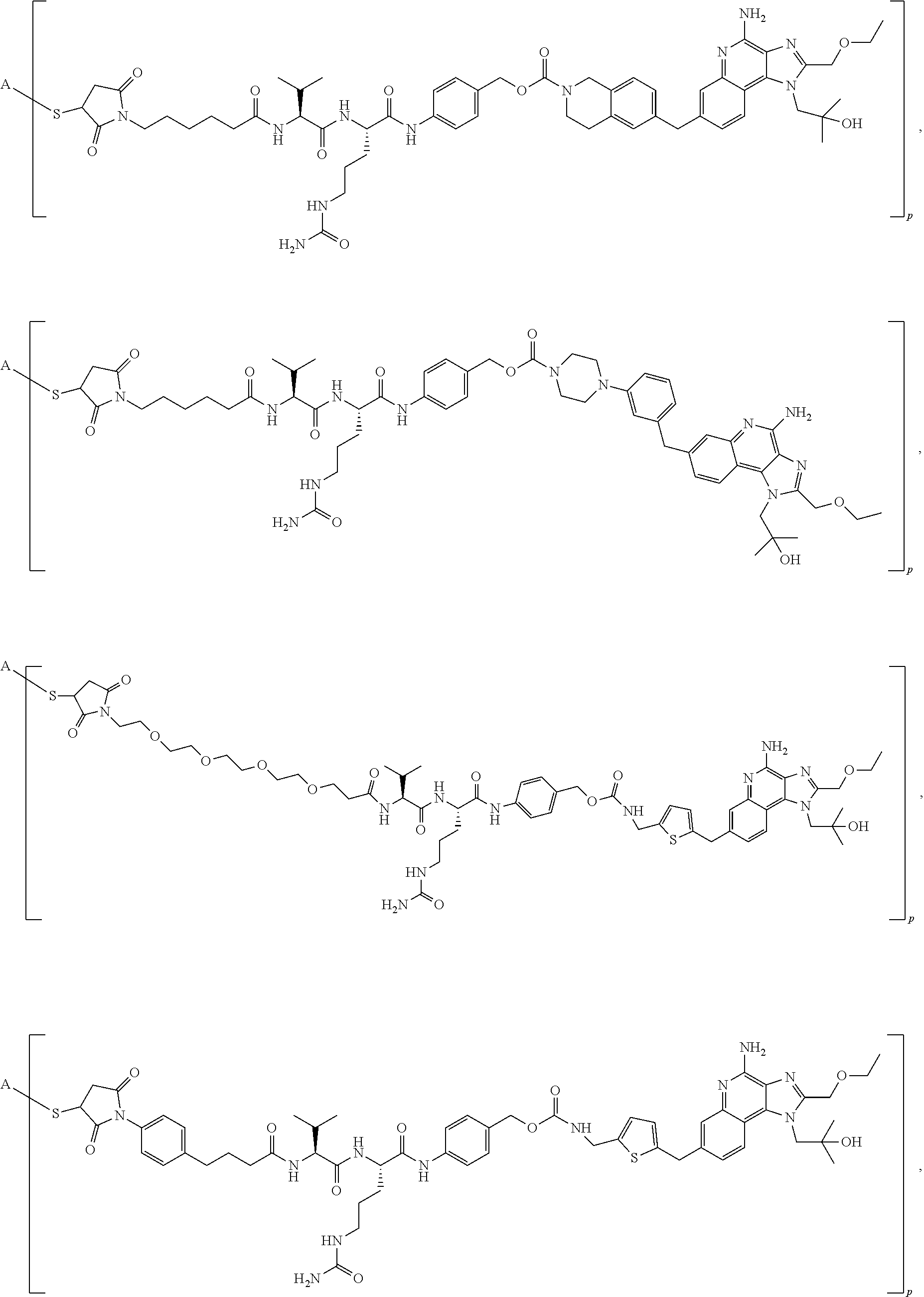 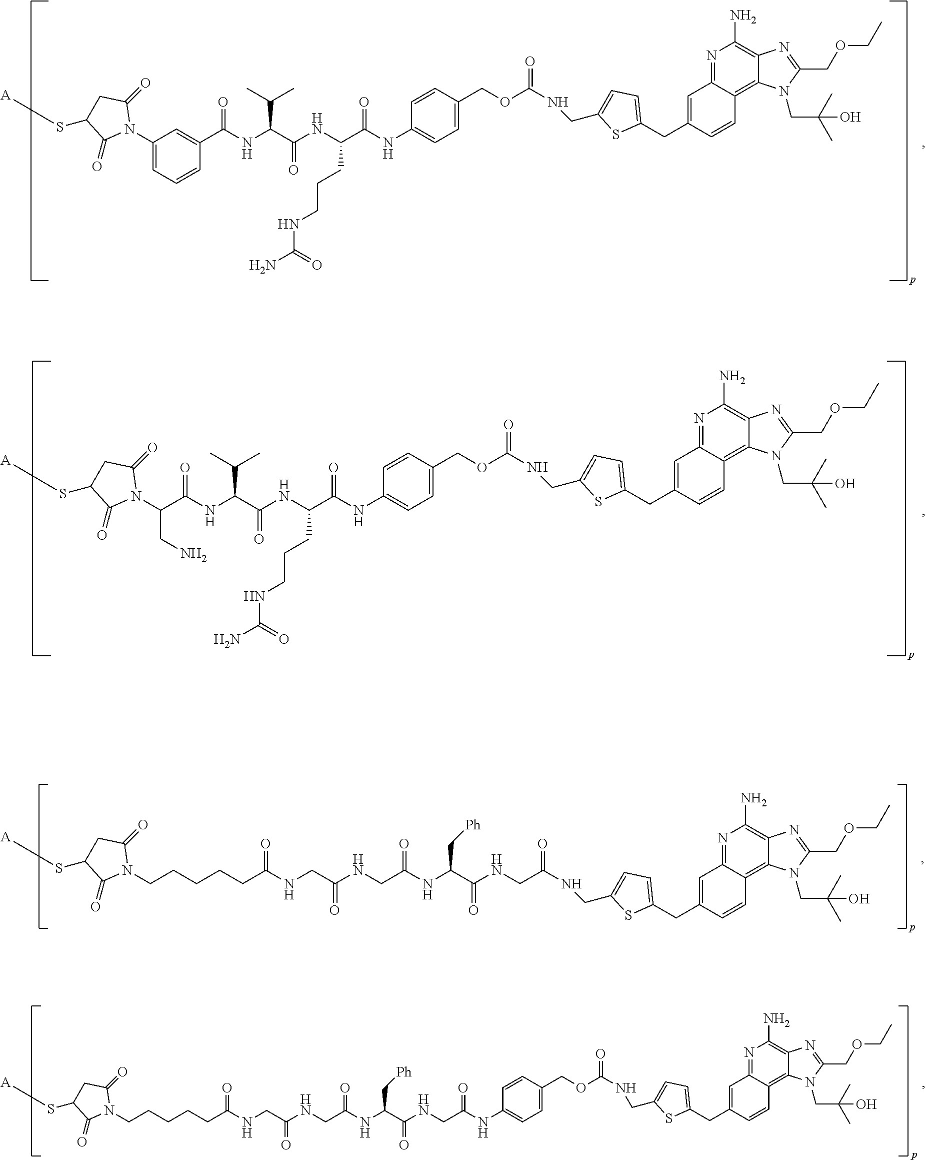 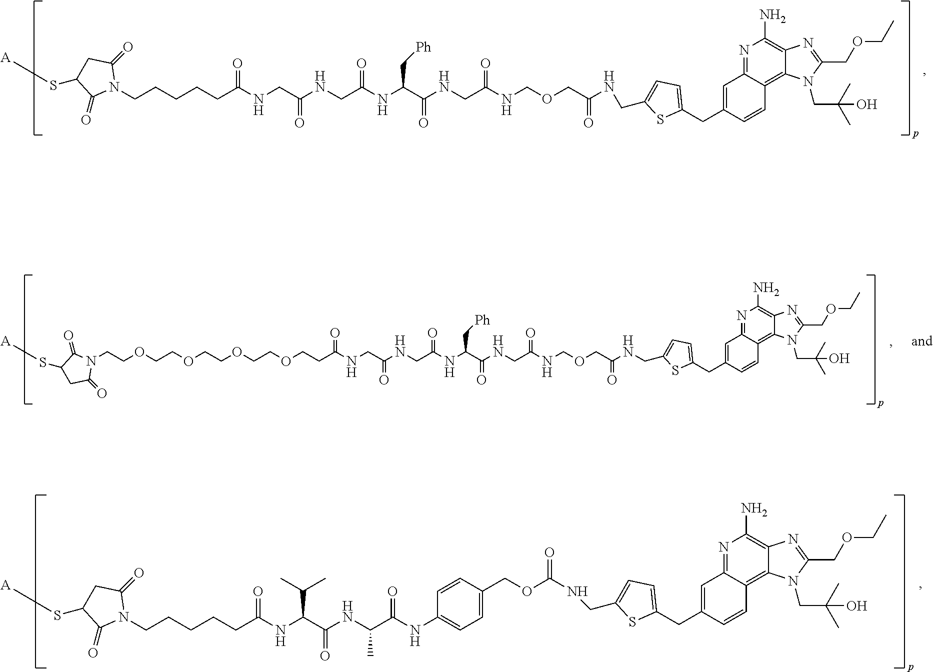
or a pharmaceutically acceptable salt thereof.

216. The conjugate compound of claim 202, wherein the targeting moiety comprises an antibody or antigen binding fragment thereof, optionally the antibody binds to HER2 or B7-H4.

217. A linker-payload compound having Formula (Ia): 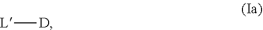
or a pharmaceutically acceptable salt thereof, wherein
L′ is a linker precursor;
D is a payload unit of Formula (II): 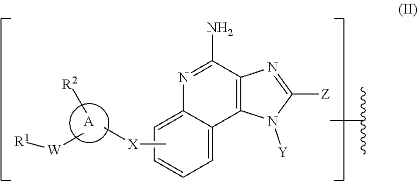
wherein:
X is selected from the group consisting of —O—, —S—, —NH—, and —(CH₂)ᵢ—, —(X¹)NC(O)—, —(X¹)NS(O)₂—, —C(O)N(X¹)— and —S(O)₂N(X¹)—, wherein —NH— and —(CH₂)ᵢ— are optionally substituted with one or more groups independently selected from halogen, hydroxyl, cyano, amino or alkyl;
each X¹ is independently hydrogen, alkyl, alkenyl, or haloalkyl;
Ring A is cycloalkyl, heterocyclyl, aryl or heteroaryl;
W is selected from the group consisting of a direct bond, alkyl, cycloalkyl, heterocyclyl, *—W¹-alkyl-, *-alkyl-W¹—, and *-alkyl-W¹-alkyl-, wherein the alkyl, cycloalkyl, heterocyclyl, and alkyl in *—W¹-alkyl-, *-alkyl-W¹—, and *-alkyl-W¹-alkyl are optionally substituted with one or more groups independently selected from halogen, hydroxyl, cyano, amino, alkyl and —C(O)OR^{a}, and wherein * end of W is connected to Ring A;
W¹ is —O—, —NR^{a}—, —C(O)—, —C(O)NR^{a}—, —NR^{a}C(O)—, —OC(O)NR^{a}—, —NR^{a}C(O)O—, or —NR^{a}C(O)NR^{a}—,
each R^{a} is independently hydrogen, alkyl, or haloalkyl;
R¹ is hydrogen, —N(R^{b})₂, hydroxyl or SH;
each R^{b} is independently hydrogen, alkyl, or haloalkyl, or
two R^{b} taken together with the nitrogen atom to which they are bound form a heterocyclyl;
R² is hydrogen, halogen, hydroxyl, cyano, amino, alkyl, alkenyl, alkynyl, alkoxyl, haloalkyl, —OR^{e}, —OC(O)R^{e}, —SR^{e}, —B(OH)₂, —NO₂, —CHO, —C(═NOR^{e})(R^{e}), —R²⁻¹—N(R^{e})₂, —R²⁻¹—N(R^{e})C(O)R^{e}, —R²⁻¹—N(R^{e})S(O)₂R^{e}, —R²⁻¹—N(R^{e})P(O)₂R^{e}, —R²⁻¹—C(O)OR^{e}, —R²⁻¹—C(O)N(R^{e})₂, —R²⁻¹—S(O)₂N(R^{e})₂, —R²⁻¹—P(O)₂N(R^{e})₂, —OC(O)NR^{e}, or —NC(O)NR^{e},
each R²⁻¹ is independently null or alkyl;
each R^{e} is independently hydrogen, alkyl, or haloalkyl;
Y is —Y¹—Y²—Y³, wherein
Y¹ is a direct bond or —(CH₂)ₘ-Q¹-(CH₂)ₙ—O—*, wherein —(CH₂)ₘ— and —(CH₂)ₙ— are optionally substituted with one or more groups independently selected from halogen, hydroxyl, alkyl or haloalkyl, and * end of Y¹ is connected to Y²;
Y² is a direct bond or —(CH₂)ₛ-Q²-(CH₂)ₜ—NR^{c}—**, wherein —(CH₂)ₛ— and —(CH₂)ₜ— are optionally substituted with one or more groups independently selected from halogen, hydroxyl, alkyl or haloalkyl, and ** end of Y² is connect to Y³;
Y³ is selected from the group consisting of hydrogen, alkyl, -alkyl-aryl, -alkyl-N(R^{b})₂, —C(O)-alkyl, —C(O)-alkyl-N(R^{b})₂, and —S(O)₂-alkyl;
Q¹ and Q² are each independently selected from a direct bond, cycloalkyl, heterocyclyl, aryl, or heteroaryl, wherein the cycloalkyl, heterocyclyl, aryl, and heteroaryl are optionally substituted with one or more groups independently selected from halogen, hydroxyl, cyano, amino, or alkyl;
R^{c} is hydrogen or alkyl, or
R^{c} and Y³ taken together with the atoms to which they are bound form a heterocyclyl optionally substituted with one or more groups independently selected from halogen, hydroxyl, cyano, amino or alkyl;
Z is selected from the group consisting of alkyl, alkenyl, alkynyl, heteroalkyl, heteroalkenyl and heteroalkynyl, each of which is optionally substituted with one or more R^{d};
R^{d} is selected from the group consisting of halogen, acyl, alkyl, alkenyl, alkynyl, cycloalkyl and —O-cycloalkyl;
i is 0, 1, 2, 3, 4, 5 or 6;
m is 0, 1,2, 3, 4 or 5;
n is 0, 1, 2, 3, 4 or 5;
s is 0, 1, 2, 3, 4 or 5; and
t is 0, 1,2,3,4 or 5.

218. The linker-payload compound of claim 217, selected from the group consisting of: 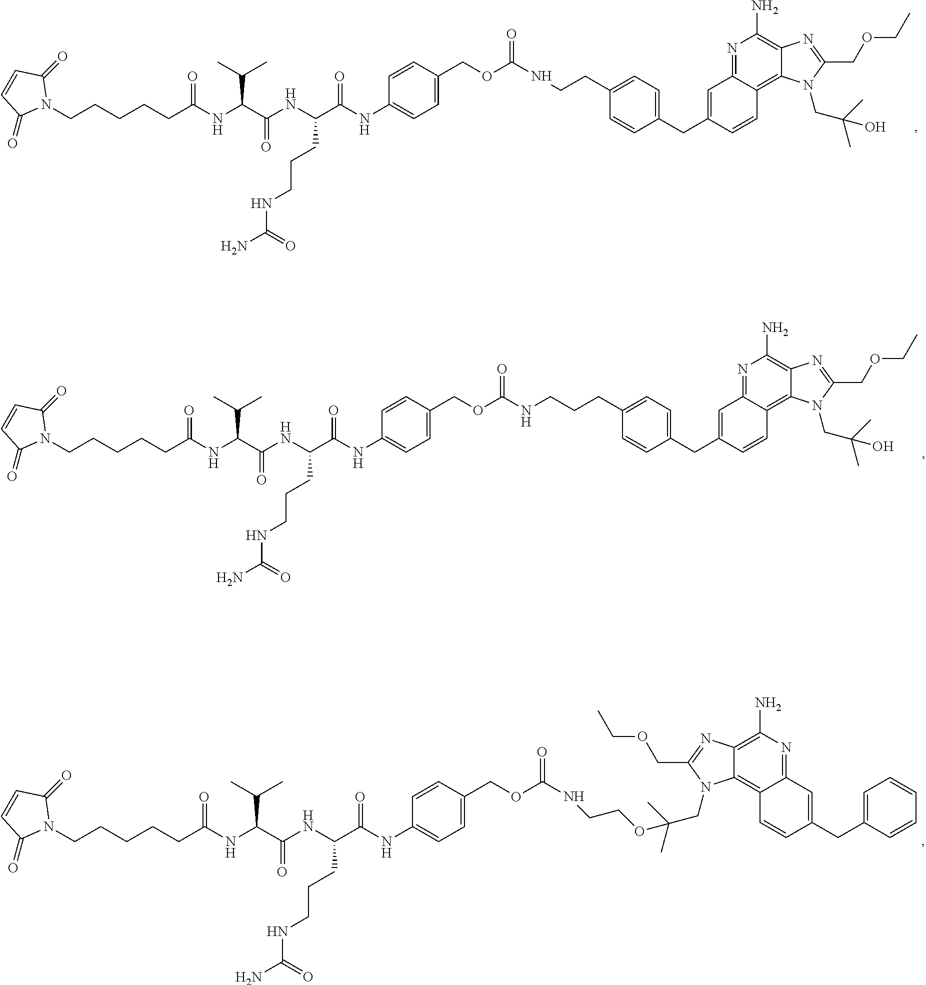 [SMILES: CCOCc1nc2c(N)nc3cc(Cc4ccc(CCNC(=O)OCc5ccc(NC(=O)[C@H](CCCNC(N)=O)NC(=O)[C@@H](NC(=O)CCCCCN6C(=O)C=CC6=O)C(C)C)cc5)cc4)ccc3c2n1CC(C)(C)O ; CCOCc1nc2c(N)nc3cc(Cc4ccc(CCCNC(=O)OCc5ccc(NC(=O)[C@H](CCCNC(N)=O)NC(=O)[C@@H](NC(=O)CCCCCN6C(=O)C=CC6=O)C(C)C)cc5)cc4)ccc3c2n1CC(C)(C)O ; CCOCc1nc2c(N)nc3cc(Cc4ccccc4)ccc3c2n1CC(C)(C)OCCNC(=O)OCc1ccc(NC(=O)[C@H](CCCNC(N)=O)NC(=O)[C@@H](NC(=O)CCCCCN2C(=O)C=CC2=O)C(C)C)cc1] 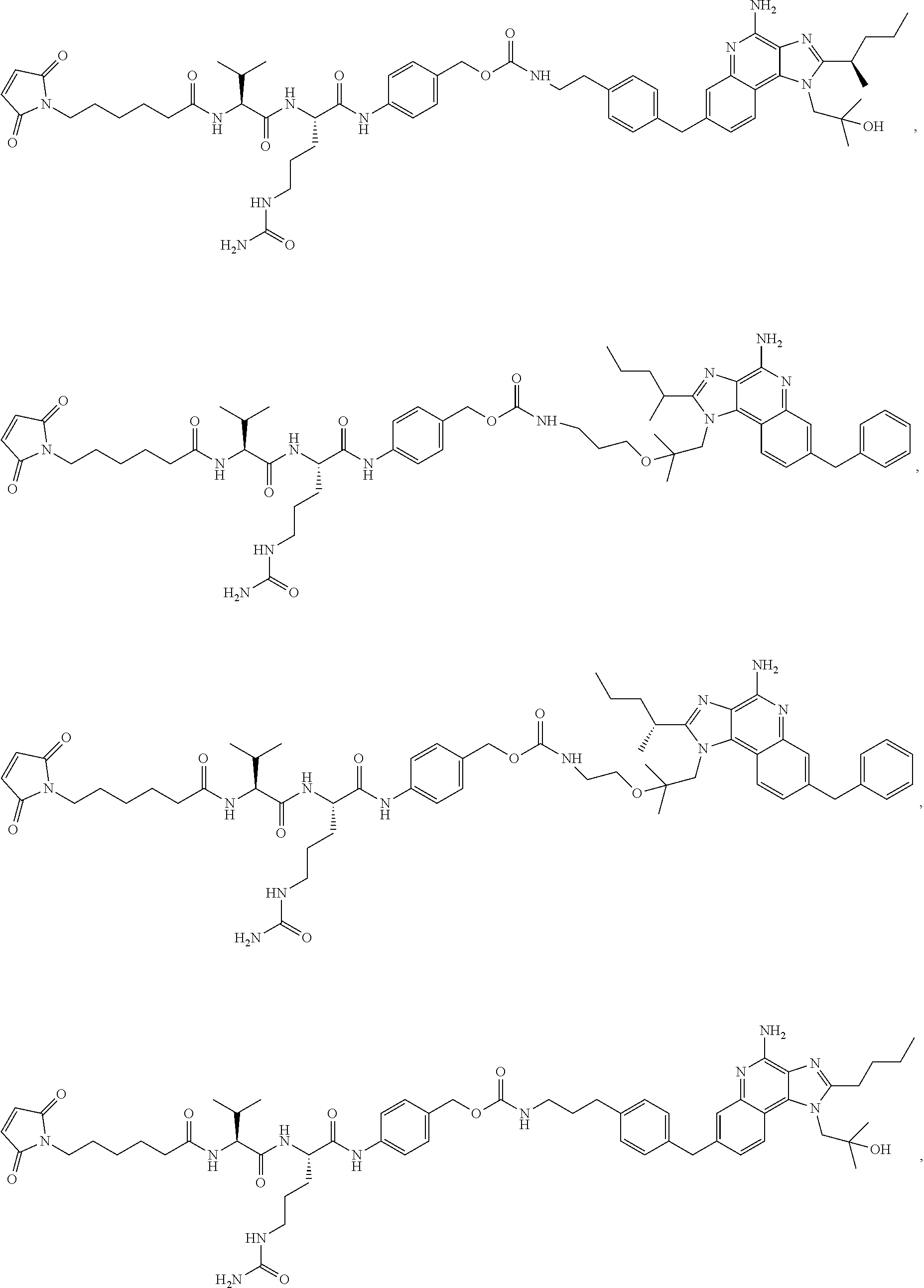 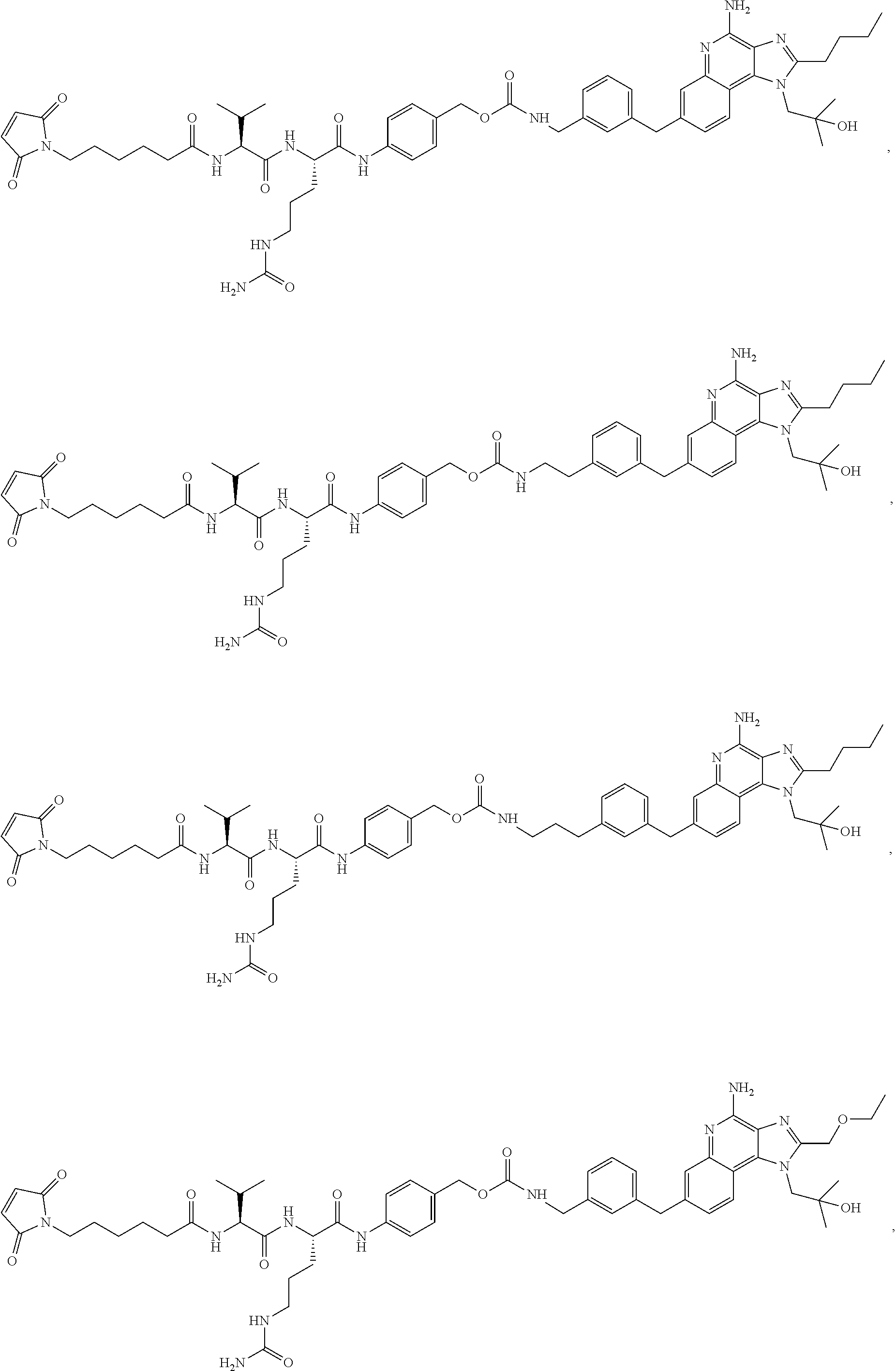 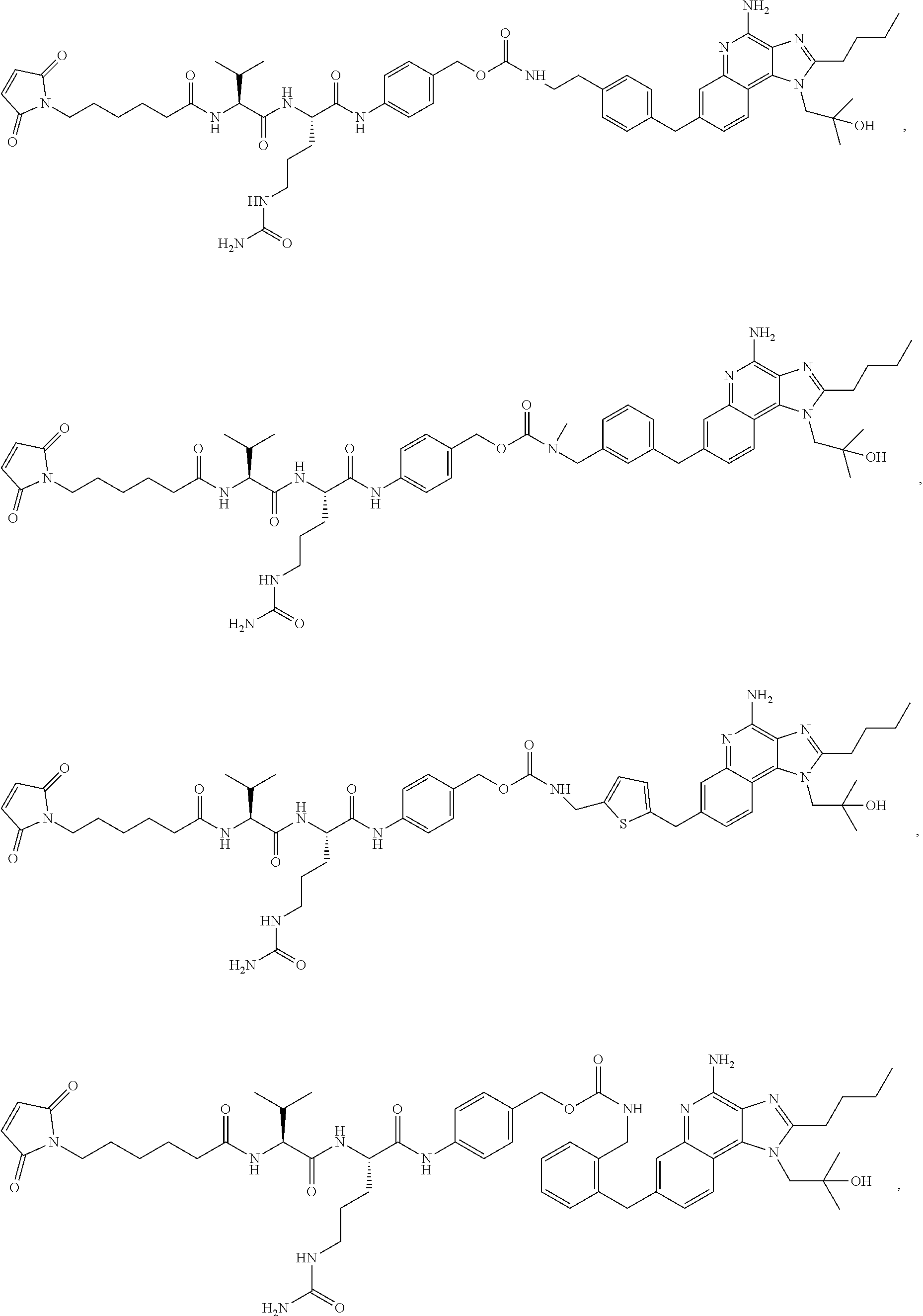 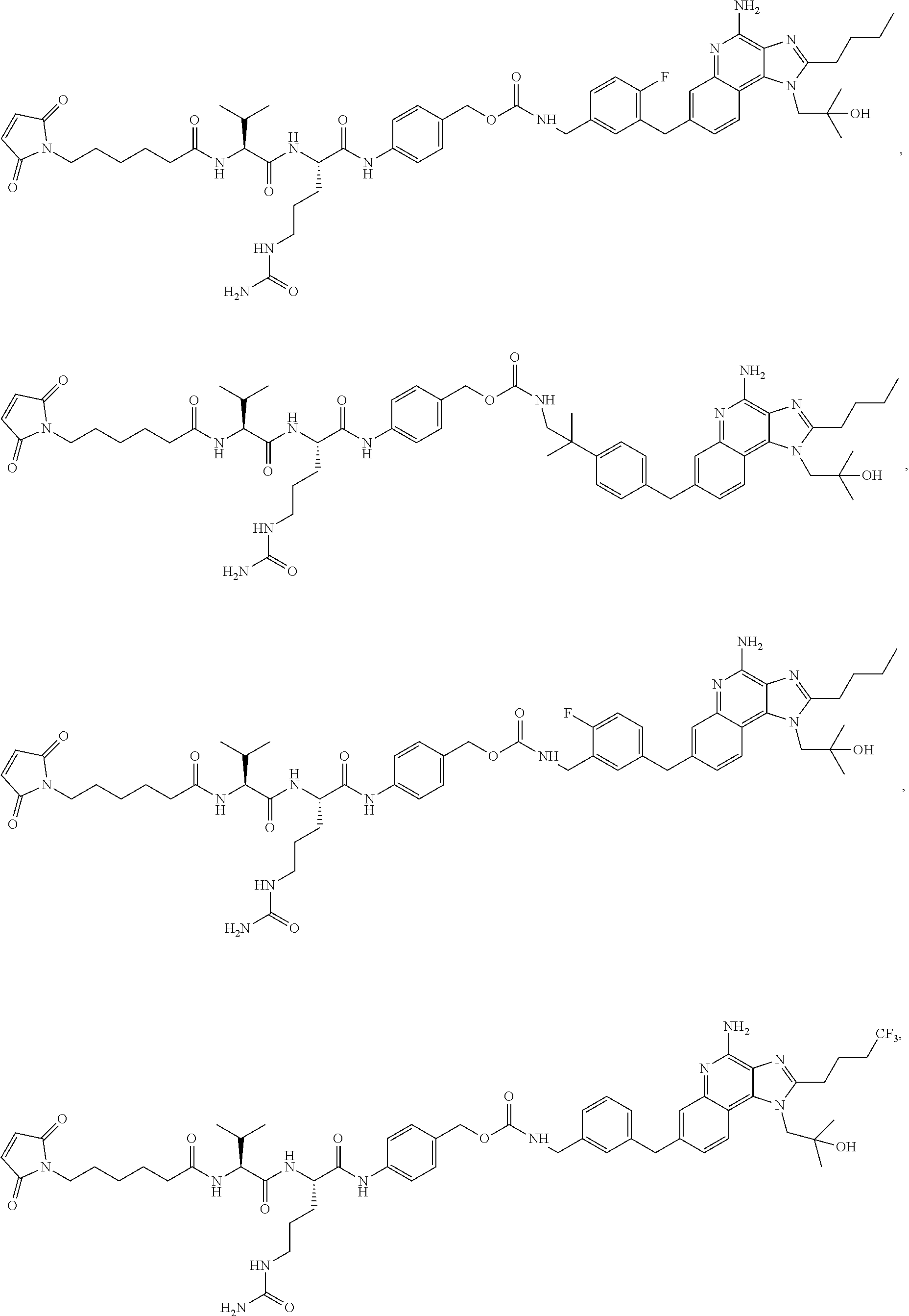 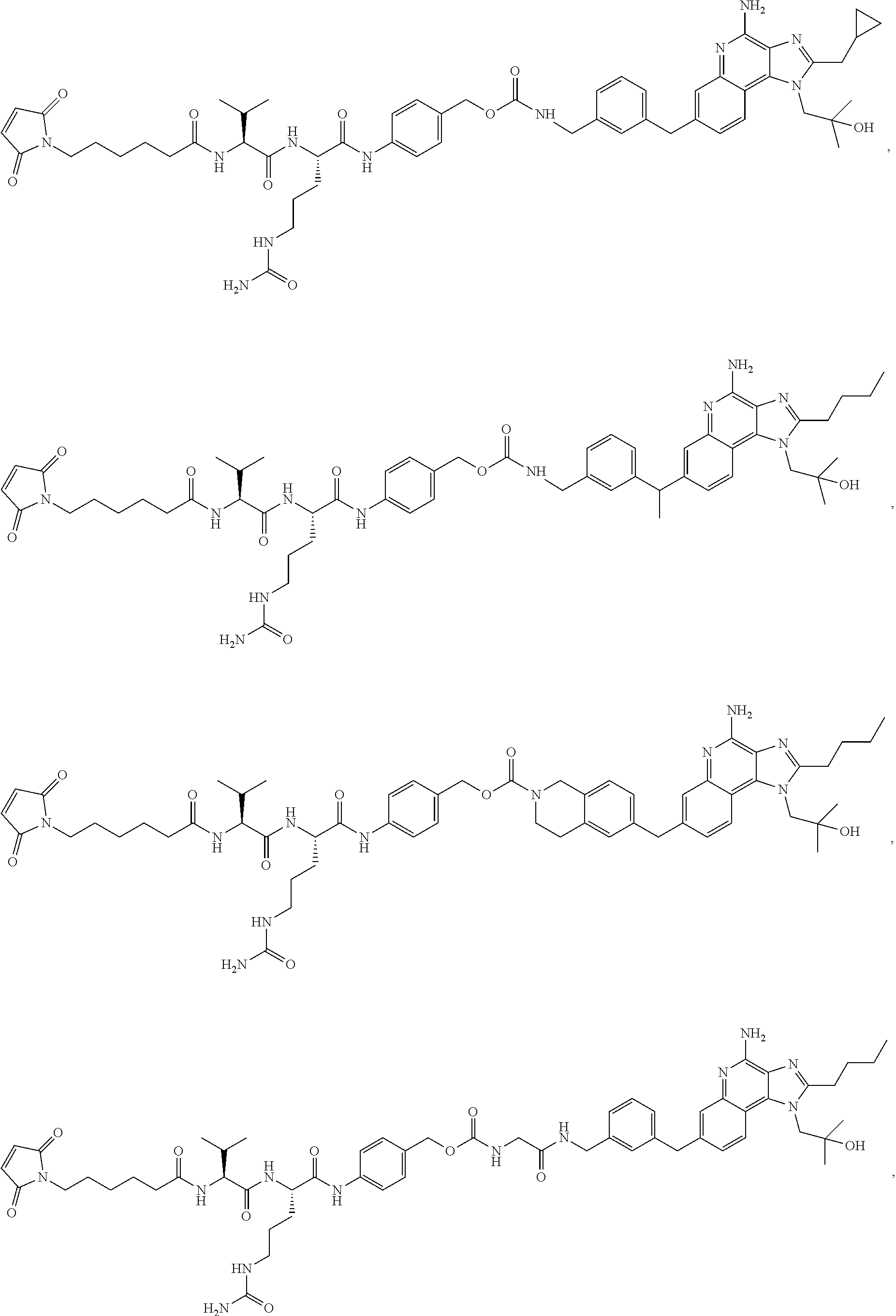 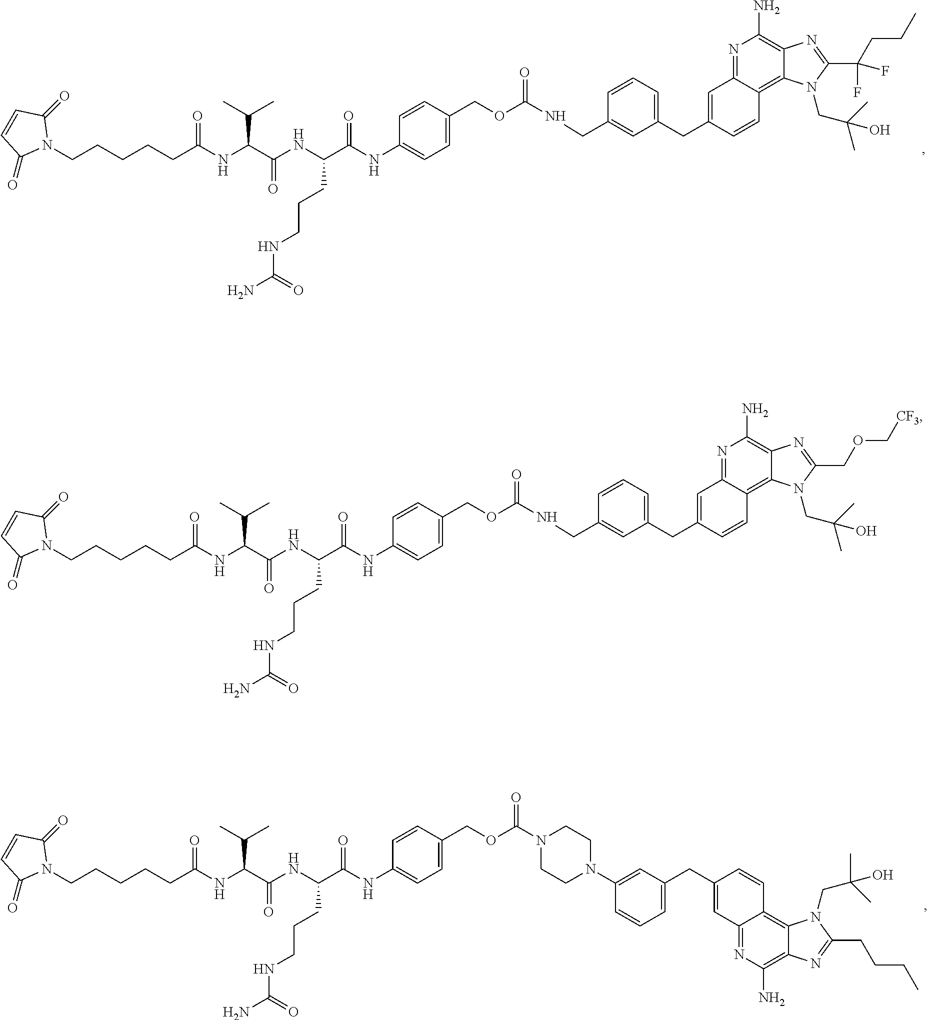 [SMILES: CCCC(F)(F)c1nc2c(N)nc3cc(Cc4cccc(CNC(=O)OCc5ccc(NC(=O)[C@H](CCCNC(N)=O)NC(=O)[C@@H](NC(=O)CCCCCN6C(=O)C=CC6=O)C(C)C)cc5)c4)ccc3c2n1CC(C)(C)O ; CC(C)[C@H](NC(=O)CCCCCN1C(=O)C=CC1=O)C(=O)N[C@@H](CCCNC(N)=O)C(=O)Nc1ccc(COC(=O)NCc2cccc(Cc3ccc4c(c3)nc(N)c3nc(COCC(F)(F)F)n(CC(C)(C)O)c34)c2)cc1 ; CCCCc1nc2c(N)nc3cc(Cc4cccc(N5CCN(C(=O)OCc6ccc(NC(=O)[C@H](CCCNC(N)=O)NC(=O)[C@@H](NC(=O)CCCCCN7C(=O)C=CC7=O)C(C)C)cc6)CC5)c4)ccc3c2n1CC(C)(C)O] 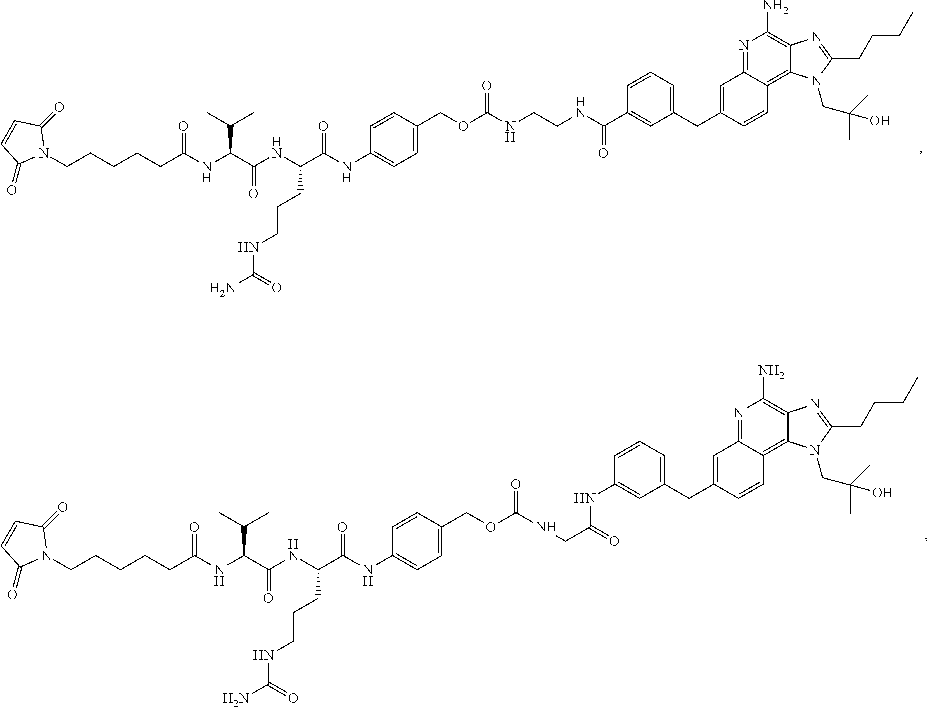 [SMILES: CCCCc1nc2c(N)nc3cc(Cc4cccc(C(=O)NCCNC(=O)OCc5ccc(NC(=O)[C@H](CCCNC(N)=O)NC(=O)[C@@H](NC(=O)CCCCCN6C(=O)C=CC6=O)C(C)C)cc5)c4)ccc3c2n1CC(C)(C)O ; CCCCc1nc2c(N)nc3cc(Cc4cccc(NC(=O)CNC(=O)OCc5ccc(NC(=O)[C@H](CCCNC(N)=O)NC(=O)[C@@H](NC(=O)CCCCCN6C(=O)C=CC6=O)C(C)C)cc5)c4)ccc3c2n1CC(C)(C)O] 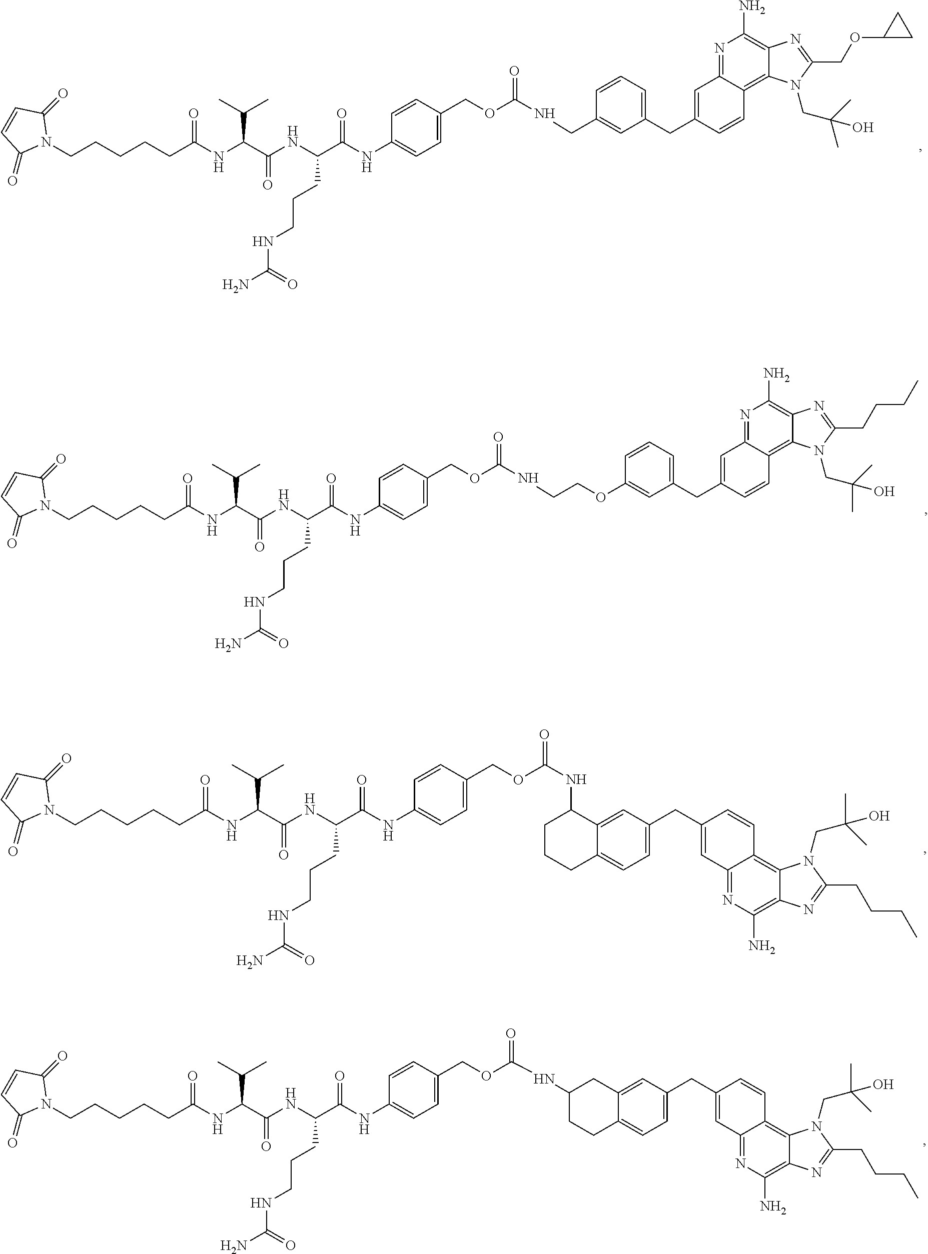 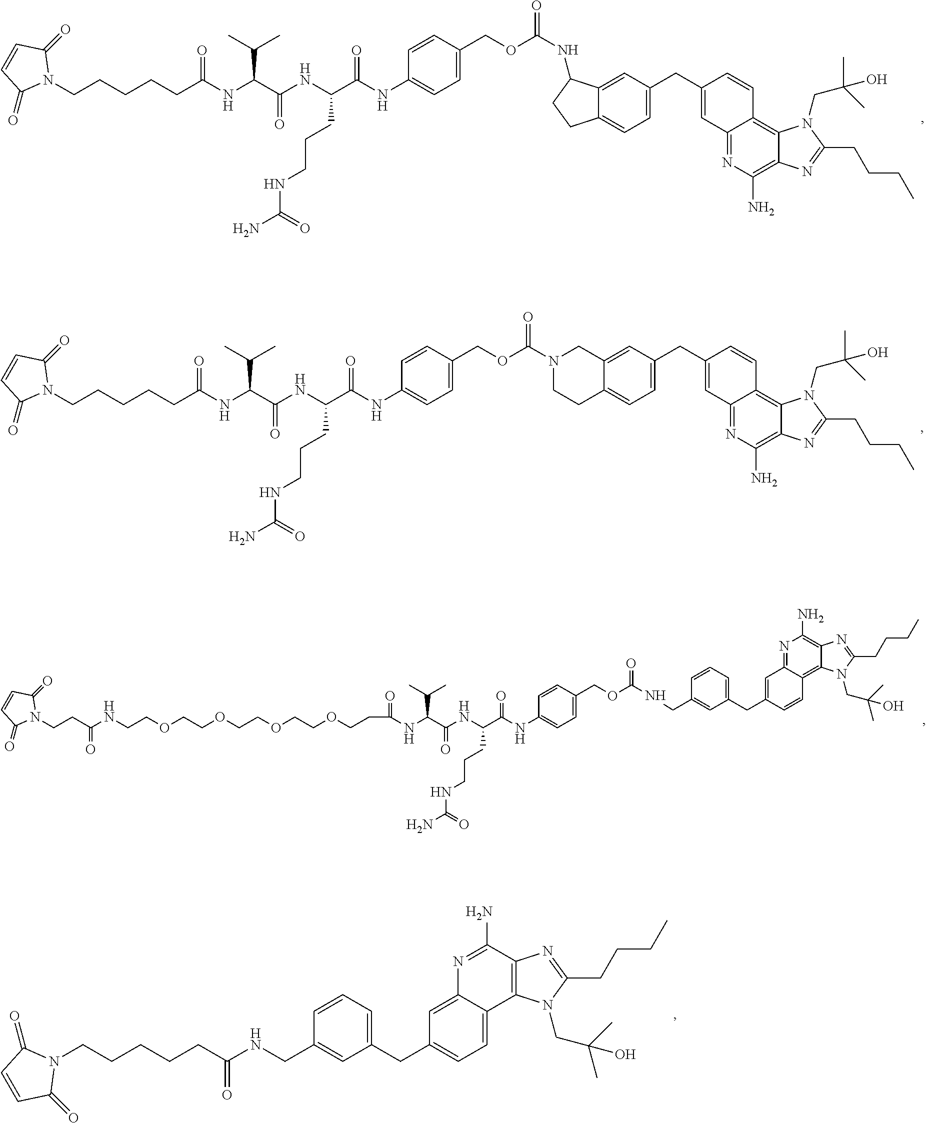 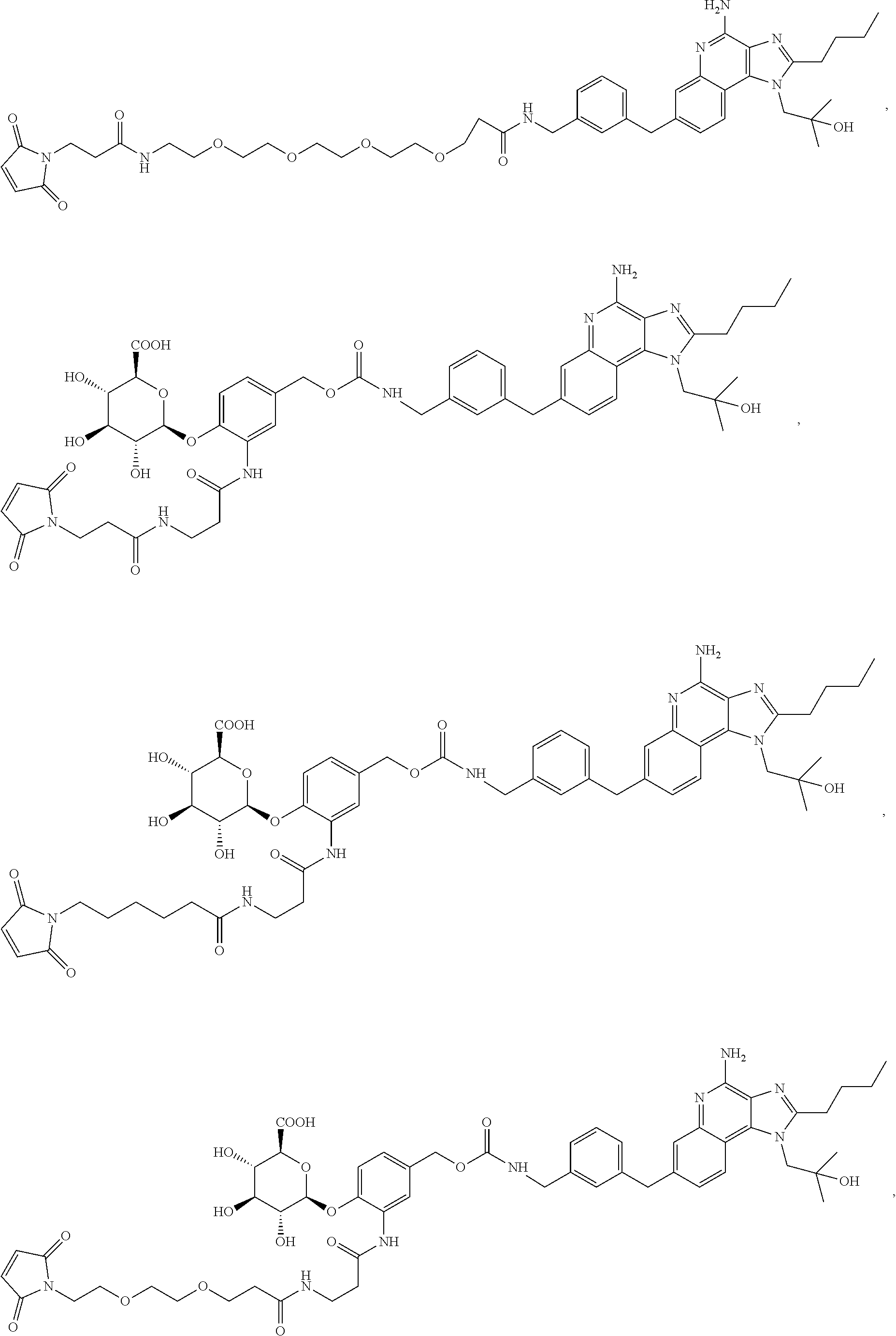 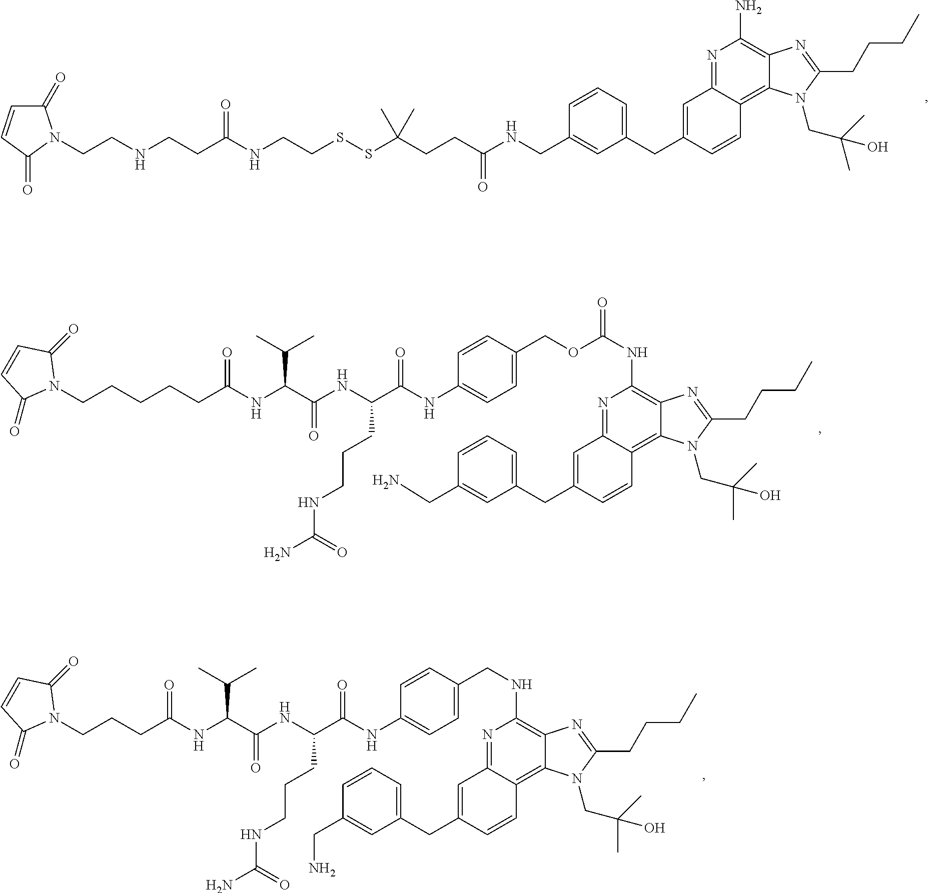 [SMILES: CCCCc1nc2c(N)nc3cc(Cc4cccc(CNC(=O)CCC(C)(C)SSCCNC(=O)CCNCCN5C(=O)C=CC5=O)c4)ccc3c2n1CC(C)(C)O ; CCCCc1nc2c(NC(=O)OCc3ccc(NC(=O)[C@H](CCCNC(N)=O)NC(=O)[C@@H](NC(=O)CCCCCN4C(=O)C=CC4=O)C(C)C)cc3)nc3cc(Cc4cccc(CN)c4)ccc3c2n1CC(C)(C)O ; CCCCc1nc2c(NCc3ccc(NC(=O)[C@H](CCCNC(N)=O)NC(=O)[C@@H](NC(=O)CCCN4C(=O)C=CC4=O)C(C)C)cc3)nc3cc(Cc4cccc(CN)c4)ccc3c2n1CC(C)(C)O] 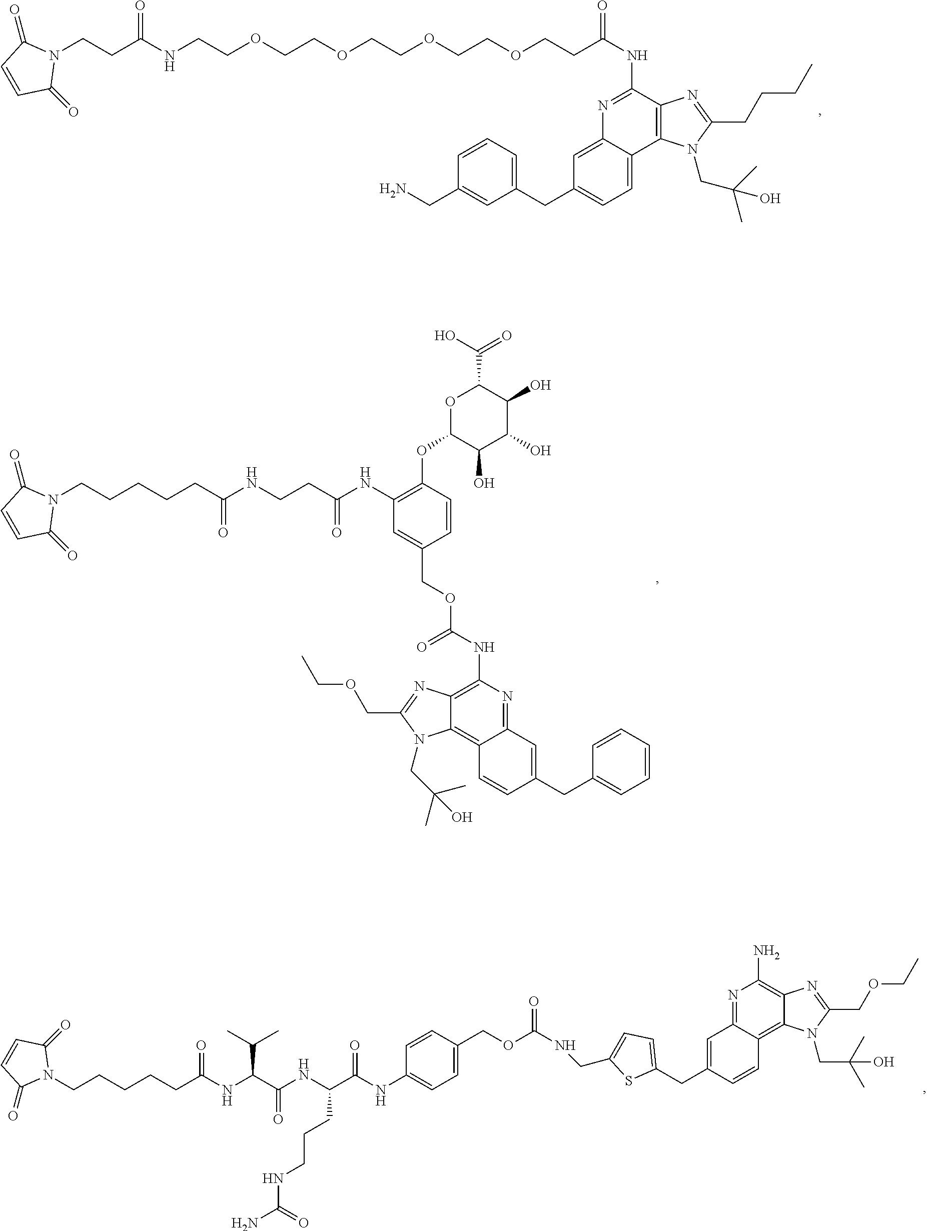 [SMILES: CCCCc1nc2c(NC(=O)CCOCCOCCOCCOCCNC(=O)CCN3C(=O)C=CC3=O)nc3cc(Cc4cccc(CN)c4)ccc3c2n1CC(C)(C)O ; CCOCc1nc2c(NC(=O)OCc3ccc(O[C@@H]4O[C@H](C(=O)O)[C@@H](O)[C@H](O)[C@H]4O)c(NC(=O)CCNC(=O)CCCCCN4C(=O)C=CC4=O)c3)nc3cc(Cc4ccccc4)ccc3c2n1CC(C)(C)O ; CCOCc1nc2c(N)nc3cc(Cc4ccc(CNC(=O)OCc5ccc(NC(=O)[C@H](CCCNC(N)=O)NC(=O)[C@@H](NC(=O)CCCCCN6C(=O)C=CC6=O)C(C)C)cc5)s4)ccc3c2n1CC(C)(C)O] 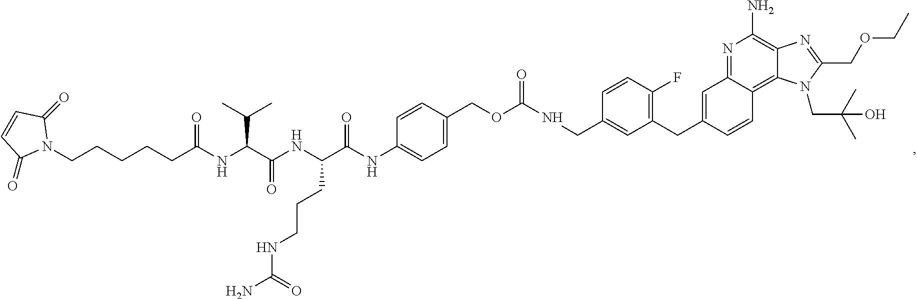 [SMILES: CCOCc1nc2c(N)nc3cc(Cc4cc(CNC(=O)OCc5ccc(NC(=O)[C@H](CCCNC(N)=O)NC(=O)[C@@H](NC(=O)CCCCCN6C(=O)C=CC6=O)C(C)C)cc5)ccc4F)ccc3c2n1CC(C)(C)O] 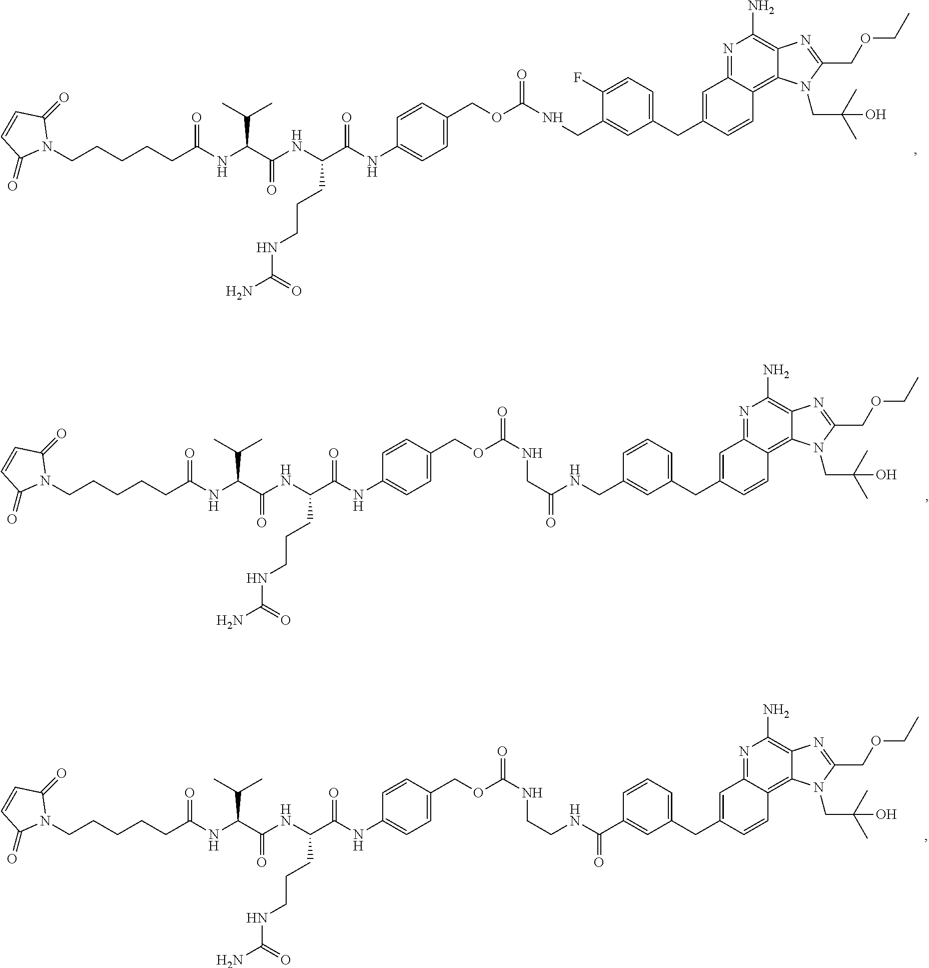 [SMILES: CCOCc1nc2c(N)nc3cc(Cc4ccc(F)c(CNC(=O)OCc5ccc(NC(=O)[C@H](CCCNC(N)=O)NC(=O)[C@@H](NC(=O)CCCCCN6C(=O)C=CC6=O)C(C)C)cc5)c4)ccc3c2n1CC(C)(C)O ; CCOCc1nc2c(N)nc3cc(Cc4cccc(CNC(=O)CNC(=O)OCc5ccc(NC(=O)[C@H](CCCNC(N)=O)NC(=O)[C@@H](NC(=O)CCCCCN6C(=O)C=CC6=O)C(C)C)cc5)c4)ccc3c2n1CC(C)(C)O ; CCOCc1nc2c(N)nc3cc(Cc4cccc(C(=O)NCCNC(=O)OCc5ccc(NC(=O)[C@H](CCCNC(N)=O)NC(=O)[C@@H](NC(=O)CCCCCN6C(=O)C=CC6=O)C(C)C)cc5)c4)ccc3c2n1CC(C)(C)O] 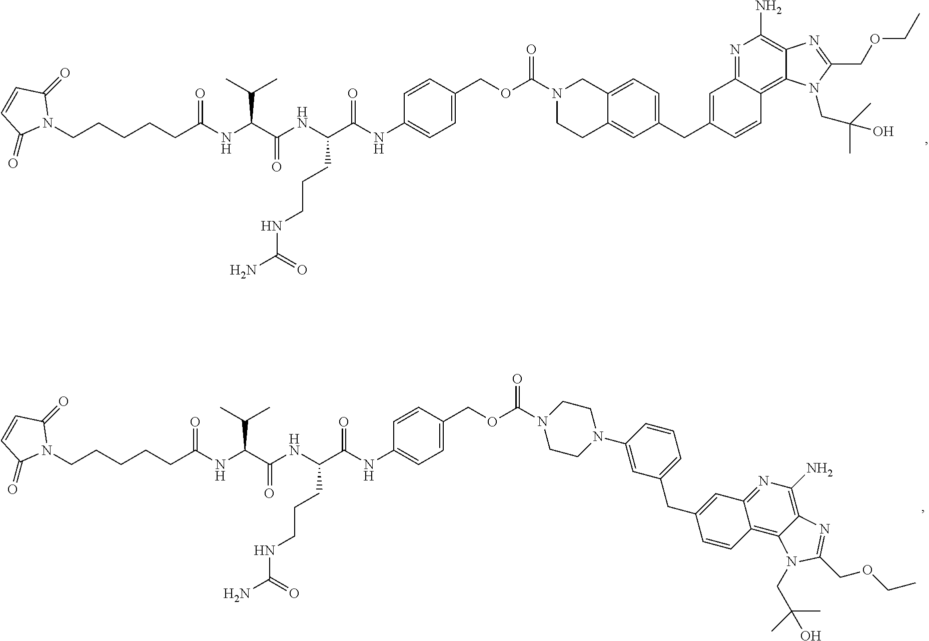 [SMILES: CCOCc1nc2c(N)nc3cc(Cc4ccc5c(c4)CCN(C(=O)OCc4ccc(NC(=O)[C@H](CCCNC(N)=O)NC(=O)[C@@H](NC(=O)CCCCCN6C(=O)C=CC6=O)C(C)C)cc4)C5)ccc3c2n1CC(C)(C)O ; CCOCc1nc2c(N)nc3cc(Cc4cccc(N5CCN(C(=O)OCc6ccc(NC(=O)[C@H](CCCNC(N)=O)NC(=O)[C@@H](NC(=O)CCCCCN7C(=O)C=CC7=O)C(C)C)cc6)CC5)c4)ccc3c2n1CC(C)(C)O] 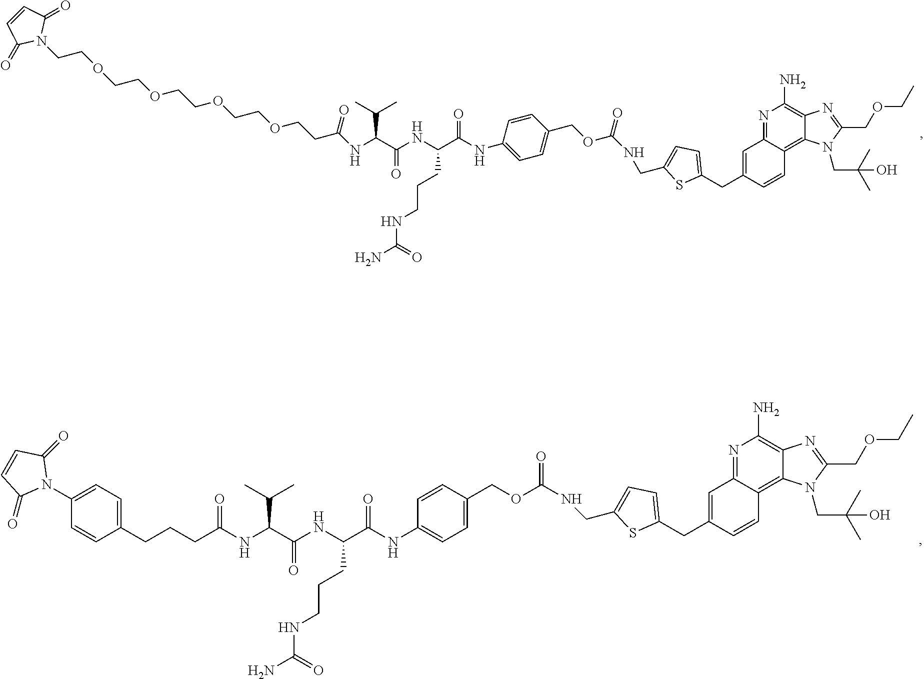 [SMILES: CCOCc1nc2c(N)nc3cc(Cc4ccc(CNC(=O)OCc5ccc(NC(=O)[C@H](CCCNC(N)=O)NC(=O)[C@@H](NC(=O)CCOCCOCCOCCOCCN6C(=O)C=CC6=O)C(C)C)cc5)s4)ccc3c2n1CC(C)(C)O ; CCOCc1nc2c(N)nc3cc(Cc4ccc(CNC(=O)OCc5ccc(NC(=O)[C@H](CCCNC(N)=O)NC(=O)[C@@H](NC(=O)CCCc6ccc(N7C(=O)C=CC7=O)cc6)C(C)C)cc5)s4)ccc3c2n1CC(C)(C)O] 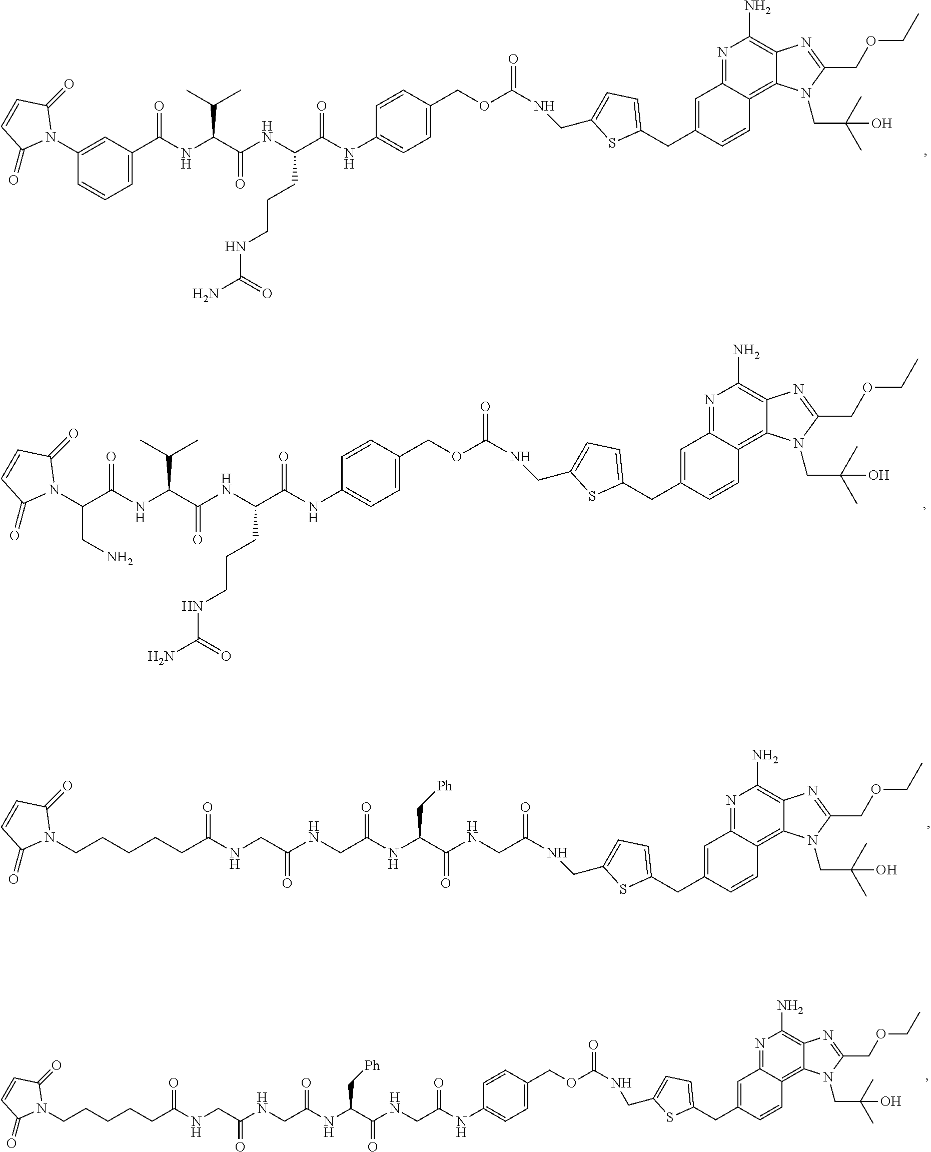 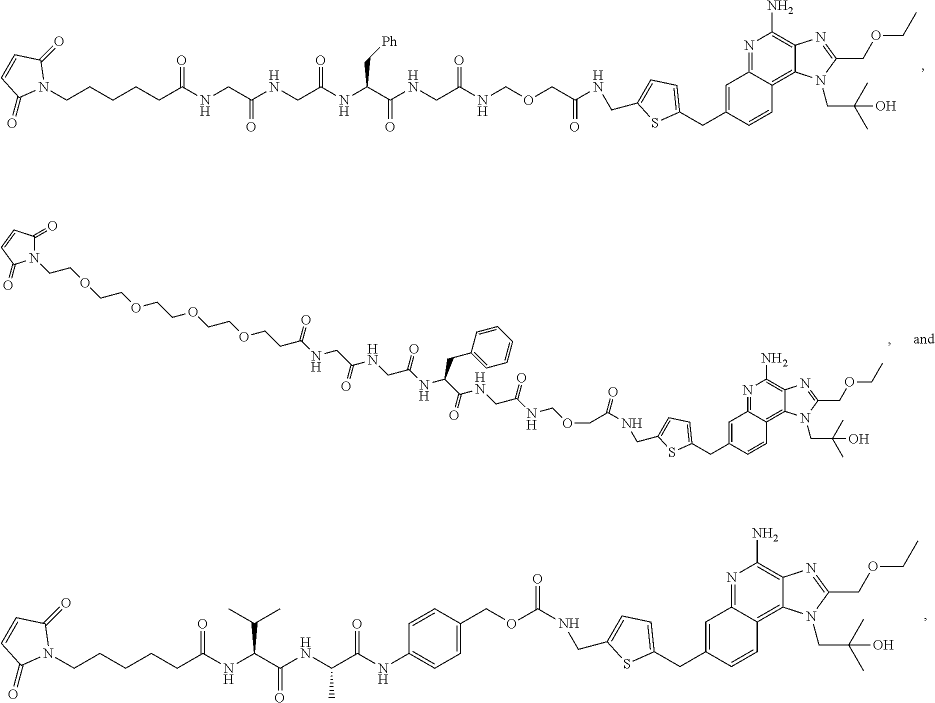 [SMILES: CCOCc1nc2c(N)nc3cc(Cc4ccc(CNC(=O)COCNC(=O)CNC(=O)[C@H](Cc5ccccc5)NC(=O)CNC(=O)CNC(=O)CCCCCN5C(=O)C=CC5=O)s4)ccc3c2n1CC(C)(C)O ; CCOCc1nc2c(N)nc3cc(Cc4ccc(CNC(=O)COCNC(=O)CNC(=O)[C@H](Cc5ccccc5)NC(=O)CNC(=O)CNC(=O)CCOCCOCCOCCOCCN5C(=O)C=CC5=O)s4)ccc3c2n1CC(C)(C)O ; CCOCc1nc2c(N)nc3cc(Cc4ccc(CNC(=O)OCc5ccc(NC(=O)[C@H](C)NC(=O)[C@@H](NC(=O)CCCCCN6C(=O)C=CC6=O)C(C)C)cc5)s4)ccc3c2n1CC(C)(C)O]
or a pharmaceutically acceptable salt thereof.

219. A payload compound having Formula (II′): 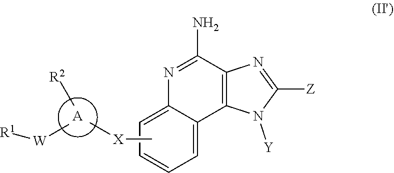
or a pharmaceutically acceptable salt thereof, wherein:
X is selected from the group consisting of —O—, —S—, —NH—, —(CH₂)ᵢ—, —(X¹)NC(O)—, —(X¹)NS(O)₂—, —C(O)N(X¹)— and —S(O)₂N(X¹)—, wherein —NH— and —(CH₂)ᵢ— are optionally substituted with one or more groups independently selected from halogen, hydroxyl, cyano, amino or alkyl;
each X¹ is independently hydrogen, alkyl, alkenyl, or haloalkyl;
Ring A is cycloalkyl, heterocyclyl, aryl or heteroaryl;
W is selected from the group consisting of a direct bond, alkyl, cycloalkyl, heterocyclyl, *—W¹-alkyl-, *-alkyl-W¹—, and *-alkyl-W¹-alkyl-, wherein the alkyl, cycloalkyl, heterocyclyl, and alkyl in *—W¹-alkyl-, *-alkyl-W¹—, and *-alkyl-W¹-alkyl are optionally substituted with one or more groups independently selected from halogen, hydroxyl, cyano, amino, alkyl and —C(O)OR^{a}, and wherein * end of W is connected to Ring A;
W¹ is —O—, —NR^{a}—, —C(O)—, —C(O)NR^{a}—, —OC(O)NR^{a}—, —NR^{a}C(O)—, —NR^{a}C(O)O—, or —NR^{a}C(O)NR^{a}—,
each R^{a} is independently hydrogen, alkyl, or haloalkyl;
R¹ is hydrogen, —N(R )₂, hydroxyl or SH;
each R^{b} is independently hydrogen, alkyl, or haloalkyl, or
two R^{b} taken together with the nitrogen atom to which they are bound form a heterocyclyl;
R² is hydrogen, halogen, hydroxyl, cyano, amino, alkyl, alkenyl, alkynyl, alkoxyl, haloalkyl, —OR^{e}, —OC(O)R^{e}, —SR^{e}, —B(OH)₂, —NO₂, —CHO, —C(═NOR^{e})(R^{e}), —R²⁻¹—N(R^{e})₂, —R²⁻¹—N(R^{e})C(O)R^{e}, —R²⁻¹—N(R^{e})S(O)₂R^{e}, —R²⁻¹—N(R^{e})P(O)₂R^{e}, —R²⁻¹—C(O)OR^{e}, —R²⁻¹—C(O)N(R^{e})₂, —R²⁻¹—S(O)₂N(R^{e})₂, —R²⁻¹—P(O)₂N(R^{e})₂, —OC(O)NR^{e}, or —NC(O)NR^{e},
each R²⁻¹ is independently null or alkyl;
each R^{e} is independently hydrogen, alkyl, or haloalkyl;
Y is —Y¹—Y²—Y³, wherein
Y¹ is a direct bond or —(CH₂)ₘ-Q¹-(CH₂)ₙ—O—*, wherein —(CH₂)ₘ— and —(CH₂)ₙ— are optionally substituted with one or more groups independently selected from halogen, hydroxyl, alkyl or haloalkyl, and * end of Y¹ is connected to Y²;
Y² is a direct bond or —(CH₂)ₛ-Q²-(CH₂)ₜ—NR^{c}—**, wherein —(CH₂)ₛ— and —(CH₂)ₜ— are optionally substituted with one or more groups independently selected from halogen, hydroxyl, alkyl or haloalkyl, and ** end of Y² is connect to Y³;
Y³ is selected from the group consisting of hydrogen, alkyl, -alkyl-aryl, -alkyl-N(R^{b})₂, —C(O)-alkyl, —C(O)-alkyl-N(R^{b})₂, and —S(O)₂-alkyl;
Q¹ and Q² are each independently selected from a direct bond, cycloalkyl, heterocyclyl, aryl, or heteroaryl, wherein the cycloalkyl, heterocyclyl, aryl, and heteroaryl are optionally substituted with one or more groups independently selected from halogen, hydroxyl, cyano, amino, or alkyl;
R^{c} is hydrogen or alkyl, or
R^{c} and Y³ taken together with the atoms to which they are bound form a heterocyclyl optionally substituted with one or more groups independently selected from halogen, hydroxyl, cyano, amino or alkyl;
Z is selected from the group consisting of alkyl, alkenyl, alkynyl, heteroalkyl, heteroalkenyl and heteroalkynyl, each of which is optionally substituted with one or more R^{d};
R^{d} is selected from the group consisting of halogen, acyl, alkyl, alkenyl, alkynyl, cycloalkyl and —O-cycloalkyl;
i is 0, 1, 2, 3, 4, 5 or 6;
m is 0, 1,2, 3, 4 or 5;
n is 0, 1, 2, 3, 4 or 5;
s is 0, 1, 2, 3, 4 or 5; and
t is 0, 1, 2, 3, 4 or 5.

220. The payload compound of claim 219, having Formula (IIa′):

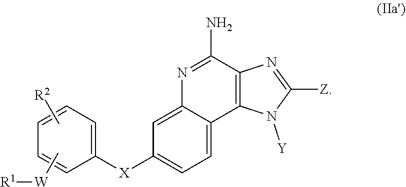

221. A pharmaceutical composition comprising the conjugate compound of claim 202, and a pharmaceutically acceptable carrier.
